(19) United States
(12) Patent Application Publication
(10) Pub. No.: US 2026/0315464 A1
(43) Pub. Date: Oct. 15, 2026

(54) COMPOSITIONS, METHODS, AND SYSTEMS FOR MODULATING SPLICING OF MSH3

(71) Applicant: REVIR THERAPEUTICS, INC., Brisbane, CA (US)

(72) Inventors: Jinxing Li, Shenzhen, Guangdong (CN); Paul R. August, Brisbane, CA (US); Mary McMahon, Brisbane, CA (US); Xing Tang, Brisbane, CA (US); Peng Yue, Brisbane, CA (US); Jacqueline Salotti, Brisbane, CA (US); Sridhar Narayan, Brisbane, CA (US)

(21) Appl. No.: 19/489,392

(22) PCT Filed: Jun. 6, 2024
(86) PCT No.: PCT/CN2024/097830

§ 371 Date: Dec. 2, 2025

Related U.S. Application Data
(60) Provisional application No. 63/506,564, filed on Jun. 6, 2023.
(60) Provisional application No. 63/580,945, filed on Sep. 6, 2023.

(51) Int. Cl. A61K 31/501; A61K 31/495; C12Q 1/6876

(52) U.S. Cl. CPC A61K 31/501; A61K 31/495; C12Q 1/6876; C12Q 2600/136

(57) ABSTRACT

Described herein are compositions, methods, and systems for modulating splicing of MSH3 pre-mRNA such that the resulting MSH3 mRNA transcript includes a poison exon. Further disclosed herein are compositions, methods, and systems for treating a disease or condition associated with a nucleotide repeat expansion in a gene.

### CROSS REFERENCE

This application claims the benefit of U.S. Provisional Application No. 63/506,564, filed on Jun. 6, 2023, and U.S. Provisional Application No. 63/580,945, filed on Sep. 6, 2023, each of which is herein incorporated by reference in its entirety.

### SEQUENCE LISTING

The instant application contains a Sequence Listing which has been submitted electronically in XML file format and is hereby incorporated by reference in its entirety. The XML copy, created on Jun. 4, 2024, is named 62236-703_601_SL.xml and is 27,601 bytes in size.

### BACKGROUND

The accumulation of nucleotide repeats in a gene can lead to manifestation of a disease or condition. For example, a nucleotide repeat expansion of nucleotides CAG is the leading cause of Huntington's disease, where a subject having the CAG repeat expansion at low abundance does not exhibit any symptoms. However, as the subject ages, the CAG nucleotide repeat accumulates (or expands), leading to manifestation of symptoms of Huntington's disease. Because of the latency associated with the nucleotide repeat expansion and the manifestation of the disease or condition, treatment options for nucleotide repeat expansion diseases or conditions are limited and ineffective.

Often times, there is no cure once the subject exhibits symptoms associated with the nucleotide repeat expansion disease or condition. Treatment options at this particular timepoint is now only limited to palliative care.

### SUMMARY

There is an unmet need for compositions, methods, and systems for treating nucleotide repeat expansion diseases or conditions. There is also an unmet need for compositions, methods, and systems to prevent or decrease nucleotide repeat expansions in a subject. This disclosure addresses this unmet need.

In one aspect, a method for modulating splicing of MSH3 pre-mRNA is provided, the method comprising contacting a cell with an agent which modulates splicing of MSH3 pre-mRNA such that an MSH3 mRNA transcript comprising a poison exon is produced, thereby modulating splicing of MSH3 pre-mRNA. In another aspect, a method for treating a disease or condition in a subject is provided, the method comprising administering to the subject an agent which modulates splicing of MSH3 pre-mRNA such that an MSH3 mRNA transcript comprising a poison exon is produced in a cell of the subject, thereby treating the disease or condition.

In some cases, the method results in decreased expression of MSH3 protein in the cell. In some cases, the method results in decreased nucleotide repeat expansion of a target gene in the cell. In some cases, the nucleotide repeat expansion comprises a trinucleotide repeat expansion. In some cases, the nucleotide repeat expansion comprises expansion of a four-nucleotide repeat, a five-nucleotide repeat, a six-nucleotide repeat, a seven-nucleotide repeat, an eight-nucleotide repeat, a nine-nucleotide repeat, a ten-nucleotide repeat, an eleven-nucleotide repeat, or a twelve-nucleotide repeat. In some cases, the nucleotide repeat expansion is in an exon of the target gene. In some cases, the nucleotide repeat expansion is in an intron of the target gene. In some cases, the nucleotide repeat expansion is in a UTR of the target gene. In some cases, the nucleotide repeat expansion is in a promoter of the target gene. In some cases, the gene is selected from the group consisting of: ATN1, HTT, AR, ATXN1, ATXN2, ATXN3, CACNA1A, ATXN7, PPP2R2B, TBP, TCF4, JPH3, DMPK, ATXN8, ZNF713, FMR1, AFF/FMR2, FXN, ZFN9, ATXN10, NOP56, C9orf72, CSTB, and any combination thereof. In some cases, the nucleotide repeat expansion in the cell comprises at least 20 repeats, at least 30 repeats, at least 40 repeats, at least 50 repeats, at least 60 repeats, at least 70 repeats, at least 80 repeats, at least 90 repeats, at least 100 repeats, at least 200 repeats, at least 500 repeats, at least 1000 repeats, at least 2000 repeats, at least 5000 repeats, at least 10,000 repeats, or at least 20,000 repeats. In some cases, the nucleotide repeat expansion in the target gene is decreased by at least about 0.1 fold, at least about 0.2 fold, at least about 0.5 fold, at least about 1.0 fold, at least about 2.0 fold, at least about 5.0 fold, at least about 10.0 fold, or at least about 100.0 fold, as compared to a level of nucleotide repeat expansion in the target gene in the absence of the agent. In some cases, the poison exon comprises a nucleic acid sequence that has at least about 80% sequence identity to the nucleic acid sequence of SEQ ID NO: 1. In some cases, the poison exon is or comprises exon 21A, or a portion thereof, of MSH3. In some cases, the poison exon is located at or within position chr5:80832901-80832994 of Human Genome Assembly hg38 of December 2013. In some cases, the poison exon comprises a nucleic acid sequence that has at least about 80% sequence identity to the nucleic acid sequence of SEQ ID NO: 2 or SEQ ID NO: 3. In some cases, the poison exon is or comprises exon 14A_1, or a portion thereof, of MSH3. In some cases, the poison exon is or comprises exon 14A_2, or a portion thereof, of MSH3. In some cases, the poison exon is located at or within position chr5:80763222-80763369 or position chr5:80763222-80767932 of Human Genome Assembly hg38 of December 2013. In some cases, the poison exon is selected from the group consisting of any one of the poison exons listed in Table 1 or Table 2. In some cases, the poison exon introduces a premature termination codon (PTC) into the MSH3 mRNA transcript. In some cases, the agent forms a complex with the MSH3 pre-mRNA and a spliceosomal RNA. In some cases, the agent increases binding of a spliceosomal RNA to the MSH3 pre-mRNA. In some cases, the spliceosomal RNA comprises a small nuclear RNA (snRNA). In some cases, the snRNA is selected from the group consisting of: U1 snRNA, U2 snRNA, U4 snRNA, U5 snRNA, U6 snRNA, and any combination thereof.

In some cases, the snRNA comprises U1 snRNA. In some cases, the agent is selected from the group consisting of: a small molecule, a polypeptide, an oligosaccharide, a polysaccharide, an oligonucleotide, an aptamer, a ribonucleoprotein (RNP), an enzyme, a nuclease, and any combination thereof. In some cases, the agent is a small molecule. In some cases, the disease or condition is a nucleotide repeat disorder. In some cases, the disease or condition is selected from the group consisting of: dentatorubropallidoluysian atrophy (DRPLA), Huntington's disease (HD), spinal and bulbar muscular atrophy (SBMA), spinocerebellar ataxia (SCA) type 1, spinocerebellar ataxia (SCA) type 2, spinocerebellar ataxia (SCA) type 3, spinocerebellar ataxia (SCA) type 6, spinocerebellar ataxia (SCA) type 7, spinocerebellar ataxia (SCA) type 8, spinocerebellar ataxia (SCA) type 10, spinocerebellar ataxia (SCA) type 12, spinocerebellar ataxia (SCA) type 17, spinocerebellar ataxia (SCA) type 31, spinocerebellar ataxia (SCA) type 36, spinocerebellar ataxia (SCA) type 37, spinocerebellar ataxia (SCA) type 50, Friedreich ataxia, fragile X syndrome (FRAXA), fragile X-associated tremor/ataxia syndrome (FXTAS), Baratela-Scott syndrome (BSS), myotonic dystrophy (DM) type 1, myotonic dystrophy (DM) type 2, amyotrophic lateral sclerosis (ALS), Kennedy's disease, fragile X E mild mental retardation (FRAXE), progressive myoclonus epilepsy (EPM1), oculopharyngodistal myopathy (OPDM) type 1, oculopharyngodistal myopathy (OPDM) type 2, oculopharyngeal myopathy with leukoencephalopathy (OPML), frontotemporal dementia (FTD), Neuronal intranuclear inclusion disease (NIID), essential tremor, global developmental delay, progressive ataxia, and elevated glutamine (GDPAG), familial myoclonic epilepsy (FAME) type 1, familial myoclonic epilepsy (FAME) type 2, familial myoclonic epilepsy (FAME) type 3, familial myoclonic epilepsy (FAME) type 4, familial myoclonic epilepsy (FAME) type 6, familial myoclonic epilepsy (FAME) type 7, premature ovarian failure (FXPOF), cerebellar ataxia, neuropathy, vestibular areflexia syndrome (CANVAS), multiple system atrophy (MSA), refractory chronic cough, Fuchs endothelial corneal dystrophy (FECD), X-linked dystonia-parkinsonism (XDP), intellectual disability (FRA12A type), Jacobsen syndrome (CBL2), and any combination thereof. In some cases, the MSH3 mRNA comprising the poison exon undergoes nonsense mediated decay (NMD).

In another aspect, a system comprising: (a) an MSH3 pre-mRNA; (b) a spliceosomal RNA; and (c) an agent which modulates splicing of the MSH3 pre-mRNA such that an MSH3 mRNA transcript comprising the poison exon is produced. In some cases, the system further comprises the MSH3 mRNA transcript comprising the poison exon. In some cases, the agent, the MSH3 pre-mRNA, and the spliceosomal RNA are in a complex. In some cases, the agent is an agent that induces binding of the spliceosomal RNA to the MSH3 pre-mRNA. In some cases, the poison exon introduces a premature termination codon into the MSH3 mRNA. In some cases, the poison exon comprises a nucleic acid sequence that has at least 80% sequence identity to the nucleic acid sequence of SEQ ID NO: 1. In some cases, the poison exon is or comprises exon 21A, or a portion thereof, of MSH3. In some cases, the poison exon is located at or within position chr5:80832901-80832994 of Human Genome Assembly hg38 of December 2013. In some cases, the poison exon comprises a nucleic acid sequence that has at least 80% sequence identity to the nucleic acid sequence of SEQ ID NO: 2 or SEQ ID NO: 3. In some cases, the poison exon is or comprises exon 14A_1, or a portion thereof, of MSH3. In some cases, the poison exon is or comprises exon 14A_2, or a portion thereof, of MSH3. In some cases, the poison exon is located at or within position chr5:80763222-80763369 or position chr5:80763222-80767932 of Human Genome Assembly hg38 of December 2013. In some cases, the poison exon is selected from the group consisting of any one of the poison exons listed in Table 1 or Table 2. In some cases, the MSH3 pre-mRNA and the spliceosomal RNA are partially complementary. In some cases, the spliceosomal RNA comprises a small nuclear RNA (snRNA). In some cases, the snRNA is selected from the group consisting of: U1 snRNA, U2 snRNA, U4 snRNA, U5 snRNA, U6 snRNA, and any combination thereof. In some cases, the snRNA comprises U1 snRNA. In some cases, the agent is selected from the group consisting of: a small molecule, a polypeptide, a carbohydrate, a monosaccharide, a oligosaccharide, a polysaccharide, an oligonucleotide, an aptamer, a biologic, a ribonucleoprotein (RNP), and any combination thereof. In some cases, the agent comprises a small molecule.

In yet another aspect, a vessel containing the system of any one of the preceding is provided. In some cases, the vessel is selected from the group consisting of: a vial, a tube, a plate, and a dish.

In yet another aspect, a cell containing the system of any one of the preceding is provided.

In yet another aspect, an mRNA transcript comprising an MSH3 mRNA and at least one poison exon is provided. In some cases, the at least one poison exon comprises a nucleic acid sequence that has at least about 80% sequence identity to the nucleic acid sequence of SEQ ID NO: 1. In some cases, the at least one poison exon is or comprises exon 21A, or a portion thereof, of MSH3. In some cases, the at least one poison exon is located at or within position chr5:80832901-80832994 of Human Genome Assembly hg38 of December 2013. In some cases, the at least one poison exon comprises a nucleic acid sequence that has at least about 80% sequence identity to the nucleic acid sequence of SEQ ID NO: 2 or SEQ ID NO: 3. In some cases, the at least one poison exon is or comprises exon 14A_1, or a portion thereof, of MSH3. In some cases, the at least one poison exon is or comprises exon 14A_2, or a portion thereof, of MSH3. In some cases, the poison exon is located at or within position chr5:80763222-80763369 or position chr5:80763222-80767932 of Human Genome Assembly hg38 of December 2013.

In some cases, the at least one poison exon is selected from the group consisting of any one of the poison exons listed in Table 1 or Table 2. In some cases, the at least one poison exon introduces a premature termination codon (PTC) into the MSH3 mRNA.

In yet another aspect, a cell comprising the mRNA transcript of any one of the preceding is provided.

In yet another aspect, a method of screening for an agent that modulates splicing of an MSH3 pre-mRNA is provided, the method comprising: (a) contacting a cell with a candidate agent; (b) detecting presence or absence of a poison exon in an MSH3 mRNA transcript; and (c) identifying the candidate agent as an agent that modulates splicing of the MSH3 pre-mRNA when the poison exon is detected in the MSH3 mRNA transcript. In some cases, the detecting comprises sequencing the MSH3 mRNA transcript. In some cases, the detecting comprises detecting an amount of the MSH3 mRNA transcript comprising the poison exon. In some cases, the detecting comprises determining a level of MSH3 protein expression. In some cases, the identifying comprises identifying the candidate agent as an agent that modulates splicing of the MSH3 pre-mRNA when the level of MSH3 protein expression is decreased as compared to a level of MSH3 protein expression in the absence of the candidate agent. In some cases, the detecting comprises determining or measuring a level of MSH3 mRNA transcript. In some cases, the identifying comprises identifying the candidate agent as an agent that modulates splicing of the MSH3 pre-mRNA when the level of MSH3 mRNA transcript is decreased as compared to a level of MSH3 mRNA transcript in the absence of the candidate agent. In some cases, the detecting comprises detecting viability of the cell. In some cases, the identifying comprises identifying the candidate agent as an agent that modulates splicing of the MSH3 pre-mRNA when viability is decreased as compared to in the absence of the candidate agent. In some cases, the method further comprises contacting the cell with an inhibitor of nonsense-mediated decay (NMD). In some cases, the inhibitor of NMD is cycloheximide. In some cases, the method further comprises determining that the poison exon leads to NMD of the MSH3 mRNA transcript when a level of the MSH3 mRNA transcript is decreased in the absence of the inhibitor of NMD, and a level of the MSH3 mRNA transcript is increased in the presence of the inhibitor of NMD.

In some cases, the method further comprises contacting a plurality of cells with a library of candidate agents, wherein each cell of the plurality of cells is contacted with a different candidate agent.

In yet another aspect, an agent is provided that modulates splicing of MSH3 pre-mRNA such that an MSH3 mRNA transcript comprising a poison exon is produced, for use in a method of treatment of the human or animal body by therapy.

In yet another aspect, an agent is provided that modulates splicing of MSH3 pre-mRNA such that an MSH3 mRNA transcript comprising a poison exon is produced, for use in a method of treatment of an expansion repeat disorder. In some cases, the expansion repeat disorder is selected from the group consisting of: wherein the disease or condition is selected from the group consisting of: dentatorubropallidoluysian atrophy (DRPLA), Huntington's disease (HD), spinal and bulbar muscular atrophy (SBMA), spinocerebellar ataxia (SCA) type 1, spinocerebellar ataxia (SCA) type 2, spinocerebellar ataxia (SCA) type 3, spinocerebellar ataxia (SCA) type 6, spinocerebellar ataxia (SCA) type 7, spinocerebellar ataxia (SCA) type 8, spinocerebellar ataxia (SCA) type 10, spinocerebellar ataxia (SCA) type 12, spinocerebellar ataxia (SCA) type 17, spinocerebellar ataxia (SCA) type 31, spinocerebellar ataxia (SCA) type 36, spinocerebellar ataxia (SCA) type 37, spinocerebellar ataxia (SCA) type 50, Friedreich ataxia, fragile X syndrome (FRAXA), fragile X-associated tremor/ataxia syndrome (FXTAS), Baratela-Scott syndrome (BSS), myotonic dystrophy (DM) type 1, myotonic dystrophy (DM) type 2, amyotrophic lateral sclerosis (ALS), Kennedy's disease, fragile X E mild mental retardation (FRAXE), progressive myoclonus epilepsy (EPM1), oculopharyngodistal myopathy (OPDM) type 1, oculopharyngodistal myopathy (OPDM) type 2, oculopharyngeal myopathy with leukoencephalopathy (OPML), frontotemporal dementia (FTD), Neuronal intranuclear inclusion disease (NIID), essential tremor, global developmental delay, progressive ataxia, and elevated glutamine (GDPAG), familial myoclonic epilepsy (FAME) type 1, familial myoclonic epilepsy (FAME) type 2, familial myoclonic epilepsy (FAME) type 3, familial myoclonic epilepsy (FAME) type 4, familial myoclonic epilepsy (FAME) type 6, familial myoclonic epilepsy (FAME) type 7, premature ovarian failure (FXPOF), cerebellar ataxia, neuropathy, vestibular areflexia syndrome (CANVAS), multiple system atrophy (MSA), refractory chronic cough, Fuchs endothelial corneal dystrophy (FECD), X-linked dystonia-parkinsonism (XDP), intellectual disability (FRA12A type), Jacobsen syndrome (CBL2), and any combination thereof.

In some cases, the agent of any of the preceding is a compound having the structure of Formula (I″), or a pharmaceutically acceptable salt or solvate thereof:

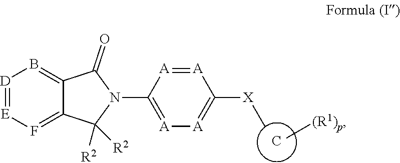

wherein,
ring C is 4 to 10-membered heterocycloalkyl or 4 to 10-membered heterocycloalkenylene;
each R¹ is independently hydrogen, halogen, —CN, —NO₂, —OH, —OR^{a}, —SH, —SR^{a}, —NR^{c}R^{d}, C₁-C₆alkyl, C₁-C₆haloalkyl, C₁-C₆hydroxyalkyl, or C₁-C₆aminoalkyl, wherein each of the alkyl, haloalkyl, hydroxyalkyl, or aminoalkyl is optionally substituted with 1 to 4 substituents independently selected from R^{e}; or
two R¹ are taken together to form an oxo; or
two R¹ are taken together to form a bond, C₁-C₃alkylene, or C₁-C₃heteroalkylene, wherein the alkylene or heteroalkylene is optionally substituted with 1 to 4 substituents independently selected from R; or
two R¹ are taken together to form a C₃-C₆cycloalkyl or 4 to 6-membered heterocycloalkyl, each of which is optionally substituted with 1 to 4 substituents independently selected from R^{e};
each R² is independently hydrogen, halogen, —OH, C₁-C₆alkyl, C₁-C₆haloalkyl, C₁-C₆hydroxyalkyl, or C₁-C₆aminoalkyl; or
X is absent, —CH₂—, —O—, —OCH₂—, —S—, or —NR³—;
R³ is hydrogen, C₁-C₃alkyl, C₁-C₃hydroxyalkyl, C₁-C₃haloalkyl, or C₃-C₆ cycloalkyl;
each A is independently N or CR⁴, wherein at least two of A are N;
each R⁴ is independently hydrogen, halogen, —CN, —NO₂, —OH, —OR^{a}, —SH, —SR^{a}, —NR^{c}R^{d}, C₁-C₆alkyl, C₁-C₆haloalkyl, C₁-C₆hydroxyalkyl, or C₁-C₆aminoalkyl;
or R³ and one of R⁴ are taken together to form a 5 or 6-membered heterocycloalkylene or 5 or 6-membered heteroarylene;
B is N or CR⁵;
D is N or CR⁶;
E is N or CR⁷;
F is N or CR⁸;
R⁵, R⁶, R⁷ or R⁸ are each independently hydrogen, halogen, —CN, —NO₂, —OH, —OR^{a}, —OCH₂R^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, C₁-C₆aminoalkyl, C₃-C₆cycloalkyl, 4 to 6 membered heterocycloalkyl, 5 to 10-membered heteroaryl, or —O—(C₁-C₆alkylene)-5 to 10-membered heteroaryl; wherein each of the alkyl, alkene, alkyne, haloalkyl, hydroxyalkyl, aminoalkyl, cycloalkyl, heterocycloalkyl, or heteroaryl is optionally substituted with 1 to 4 substituents independently selected from R^{e};
wherein at least one of R₅, R⁶, R⁷ or R⁸ is C₂-C₆alkene, C₃-C₆cycloalkyl, 4 to 6 membered heterocycloalkyl, 5 to 10-membered heteroaryl, or —O—(C₁-C₆alkylene)-5 to 10-membered heteroaryl, wherein the alkene, cycloalkyl, heterocycloalkyl and heteroaryl is optionally substituted with 1 to 4 substituents selected from R^{e};
each Rₐ is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{e} is independently halogen, oxo, —CN, —OH, —S(═O)CH₃, —S(═O)₂CH₃, —CH₂S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6-membered heterocycloalkyl; and
p is an integer from 1-9.

In some cases, the agent is a compound having the structure of Formula (I′), or a pharmaceutically acceptable salt or solvate thereof:

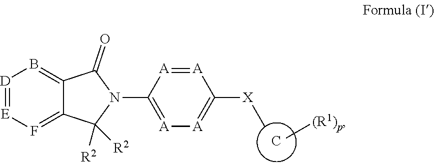

wherein,
ring C is 4 to 10-membered heterocycloalkyl or 4 to 10-membered heterocycloalkenylene;
each R¹ is independently hydrogen, halogen, —CN, —NO₂, —OH, —OR^{a}, —SH, —SR^{a}, —NR^{c}R^{d}, C₁-C₆alkyl, C₁-C₆haloalkyl, C₁-C₆hydroxyalkyl, or C₁-C₆aminoalkyl, wherein each of the alkyl, haloalkyl, hydroxyalkyl, or aminoalkyl is optionally substituted with 1 to 4 substituents independently selected from R^{e}; or
two R¹ are taken together to form an oxo; or
two R¹ are taken together to form a bond, C₁-C₃alkylene, or C₁-C₃heteroalkylene, wherein the alkylene or heteroalkylene is optionally substituted with 1 to 4 substituents independently selected from R^{e}; or
two R¹ are taken together to form a C₃-C₆cycloalkyl or 4 to 6-membered heterocycloalkyl, each of which is optionally substituted with 1 to 4 substituents independently selected from R;
each R² is independently hydrogen, halogen, —OH, C₁-C₆alkyl, C₁-C₆haloalkyl, C₁-C₆hydroxyalkyl, or C₁-C₆aminoalkyl; or
X is absent, —CH₂—, —O—, —S—, or —NR³—;
R³ is hydrogen, C₁-C₃alkyl, C₁-C₃hydroxyalkyl, C₁-C₃haloalkyl, or C₃-C₆ cycloalkyl;
each A is independently N or CR⁴, wherein at least two of A are N;
each R⁴ is independently hydrogen, halogen, —CN, —NO₂, —OH, —OR^{a}, —SH, —SR^{a}, —NR^{c}R^{d}, C₁-C₆alkyl, C₁-C₆haloalkyl, C₁-C₆hydroxyalkyl, or C₁-C₆aminoalkyl;
B is N or CR;
D is N or CR⁶;
E is N or CR⁷;
F is N or CR⁸;
R⁵ and R⁸ are each independently hydrogen, halogen, —CN, —NO₂, —OH, —OR^{a}, —SH, —SR^{a}, —S(═O)R^{a}, —S(═O)₂R^{a}, —S(═O)₂NR^{c}R^{d}, —NRcR^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, or C₁-C₆aminoalkyl; wherein each of the alkyl, haloalkyl, hydroxyalkyl, and aminoalkyl is optionally substituted with 1 to 4 substituents independently selected from R^{e};
one of R⁶ or R⁷ is a 5 to 10-membered heteroaryl optionally substituted with 1 to 4 substituents independently selected from R¹⁰; and the other of R⁶ or R⁷ is hydrogen, halogen, —CN, —NO₂, —OH, —OR^{a}, —SH, —SR, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, C₁-C₆aminoalkyl, C₃-C₆cycloalkyl, or 4 to 6 membered heterocycloalkyl; wherein each of the alkyl, alkene, alkyne, haloalkyl, hydroxyalkyl, aminoalkyl, cycloalkyl, and heterocycloalkyl is optionally substituted with 1 to 4 substituents independently selected from R^{e}; wherein R⁷ is not pyridyl substituted with two methoxy groups;
each R¹⁰ is independently halogen, —CN, —NO₂, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₃-C₆cycloalkyl, or 4 to 6-membered heterocycloalkyl, wherein each of the alkyl, haloalkyl, hydroxyalkyl, aminoalkyl, cycloalkyl and heterocycloalkyl is optionally substituted with 1 to 4-substituents independently selected from R^{e}; or
two R¹⁰ are taken together to form an oxo;
each Rₐ is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{e} is independently halogen, oxo, —CN, —OH, —S(═O)CH₃, —S(═O)₂CH₃, —CH₂S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6-membered heterocycloalkyl; and
p is an integer from 1-9.

In some cases, each R² is independently hydrogen. In some cases, D is CR⁶ or E is CR⁷. In some cases, R⁶ is a 5 to 10-membered heteroaryl optionally substituted with 1 to 4 substituents independently selected from R¹⁰; and R⁷ is hydrogen, halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, C₁-C₆aminoalkyl, C₃-C₆cycloalkyl, or 4 to 6 membered heterocycloalkyl, wherein each of the alkyl, haloalkyl, hydroxyalkyl, aminoalkyl, cycloalkyl and heterocycloalkyl is optionally substituted with 1 to 4-substituents independently selected from R^{e}. In some cases, R⁷ is hydrogen, halogen, —OH, —OR^{a}, C₁-C₆alkyl, or C₁-C₆haloalkyl. In some cases, R⁷ is hydrogen. In some cases, R⁶ is hydrogen, halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, C₁-C₆aminoalkyl, C₃-C₆cycloalkyl, or 4 to 6 membered heterocycloalkyl; wherein each of the alkyl, alkene, alkyne, haloalkyl, hydroxyalkyl, aminoalkyl, cycloalkyl, and heterocycloalkyl, wherein each of the alkyl, alkene, alkyne, haloalkyl, hydroxyalkyl, aminoalkyl, cycloalkyl and heterocycloalkyl is optionally substituted with 1 to 4-substituents independently selected from R^{e}; and R⁷ is a 5 to 10-membered heteroaryl optionally substituted with 1 to 4 substituents independently selected from R¹⁰. In some cases, R⁶ is hydrogen, halogen, —OH, —OR^{a}, C₁-C₆alkyl, or C₁-C₆haloalkyl. In some cases, R⁶ is hydrogen. In some cases, ring C is a 6-membered heterocycloalkyl. In some cases, ring C is piperidine, piperazine, or morpholine. In some cases, ring C is piperazine. In some cases, ring C is piperidine.

In some cases,

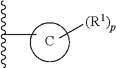

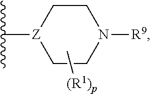

wherein,
Z is N or CH;
each R¹ is independently hydrogen, halogen, —CN, —NO₂, —OH, —OR^{a}, —SH, —SR^{a}, —NR^{c}R^{d}, C₁-C₆alkyl, C₁-C₆haloalkyl, C₁-C₆hydroxyalkyl, or C₁-C₆aminoalkyl, wherein each of the alkyl, haloalkyl, hydroxyalkyl, or aminoalkyl is optionally substituted with 1 to 4 substituents independently selected from R^{e}; or
two R¹ are taken together to form an oxo; or
two R¹ are taken together to form a bond, C₁-C₃alkylene, or C₁-C₃heteroalkylene, wherein the alkylene or heteroalkylene is optionally substituted with 1 to 4 substituents independently selected from R^{e}; or
two R¹ are taken together to form a C₃-C₆cycloalkyl or 4 to 6-membered heterocycloalkyl, each of which is optionally substituted with 1 to 4 substituents independently selected from R^{e};
R⁹ is hydrogen, C₁-C₆alkyl, C₁-C₆haloalkyl, C₃-C₆cycloalkyl, or 4 to 6-membered heterocycloalkyl, wherein each alkyl, haloalkyl, cycloalkyl and heterocycloalkyl is independently substituted with 1 to 4 substituents selected from R^{e};
each Rₐ is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e}; and
each R^{e} is independently halogen, oxo, —CN, —OH, —S(═O)CH₃, —S(═O)₂CH₃, —CH₂S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6-membered heteroaryl.

In some cases,

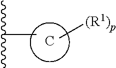

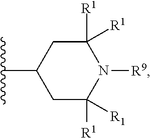

wherein,
each R^{b} is independently hydrogen, halogen, C₁-C₆alkyl, or C₁-C₆haloalkyl, wherein each of the alkyl, haloalkyl, hydroxyalkyl, or aminoalkyl is optionally substituted with 1 to 4 substituents independently selected from R^{e}; or
two R¹ are taken together to form a C₁-C₃alkylene or C₁-C₃heteroalkylene, wherein the alkylene or heteroalkylene is optionally substituted with 1 to 4 substituents independently selected from R^{e}; or
two R¹ are taken together to form a C₃-C₆cycloalkyl; and
R⁹ is hydrogen.

In some cases,

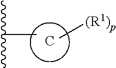

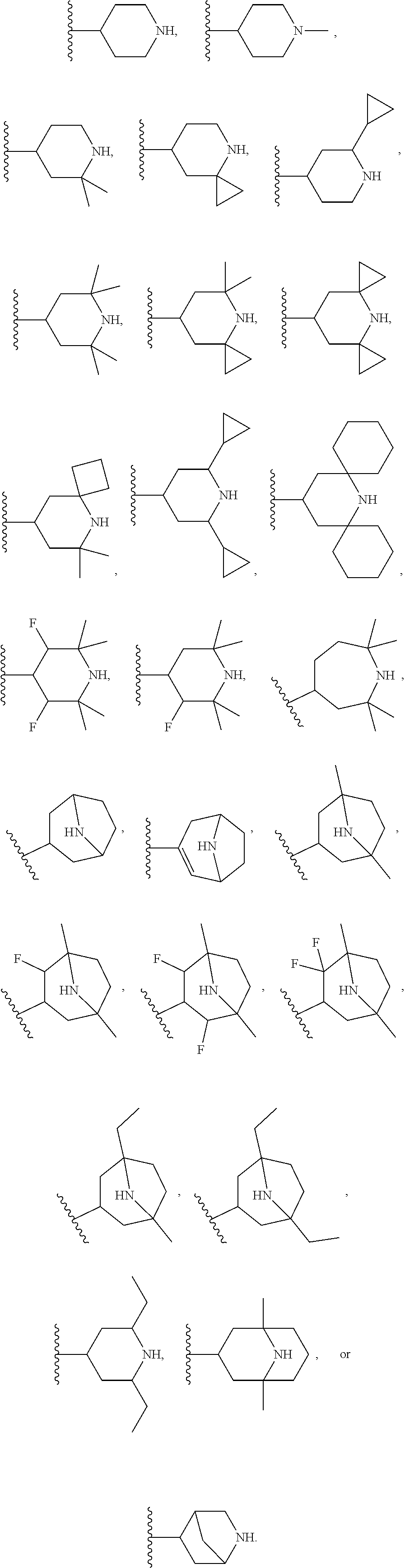

In some cases,

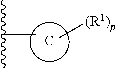

is

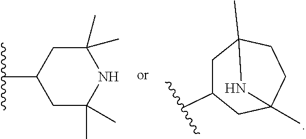 [SMILES: *C1CC(C)(C)NC(C)(C)C1 ; *C1CC2(C)CCC(C)(C1)N2]

In some cases, X is —O— or —NR³—.

In some cases, X is absent; and

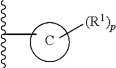

is

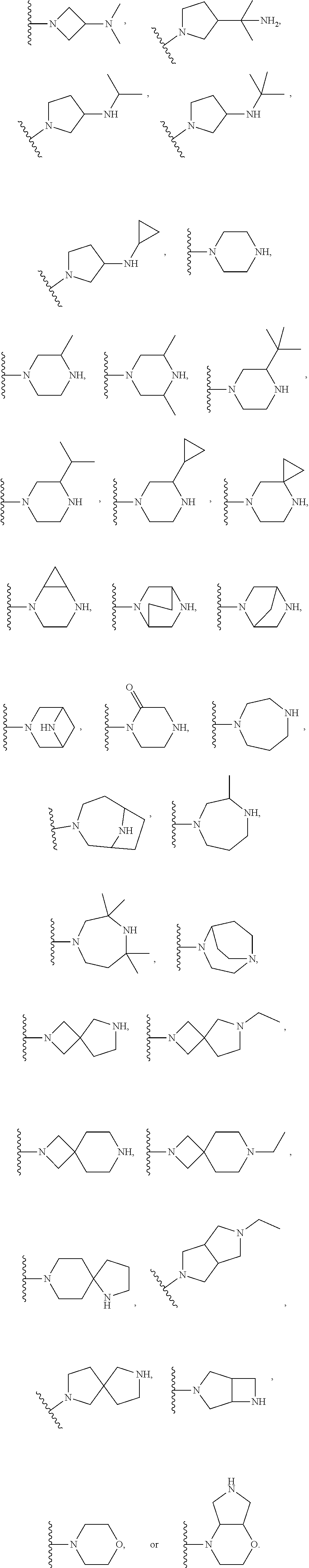

In some cases, the compound has the structure of Formula (II′), or a pharmaceutically acceptable salt or solvate thereof:

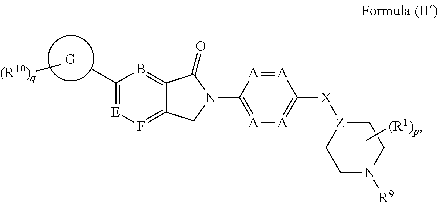

wherein,
ring G is a 5 to 10-membered heteroaryl;
each R¹ is independently hydrogen, halogen, —CN, —NO₂, —OH, —OR^{a}, —SH, —SR^{a}, —NR^{c}R^{d}, C₁-C₆alkyl, C₁-C₆haloalkyl, C₁-C₆hydroxyalkyl, or C₁-C₆aminoalkyl, wherein each of the alkyl, haloalkyl, hydroxyalkyl, or aminoalkyl is optionally substituted with 1 to 4 substituents independently selected from R^{e}; or
two R¹ are taken together to form an oxo; or
two R¹ are taken together to form a bond, C₁-C₃alkylene, or C₁-C₃heteroalkylene, wherein the alkylene or heteroalkylene is optionally substituted with 1 to 4 substituents independently selected from R^{e}; or
two R¹ are taken together to form a C₃-C₆cycloalkyl or 4 to 6-membered heterocycloalkyl, each of which is optionally substituted with 1 to 4 substituents independently selected from R^{e};
X is bond, —CH₂—, —O—, —S—, or —NR³—;
Z is CH or N, wherein when X is —O—, —S—, or —NR³—, then Z is CH;
R³ is hydrogen, C₁-C₃alkyl, C₁-C₃hydroxyalkyl, C₁-C₃haloalkyl, or C₃-C₆ cycloalkyl;
each A is independently N or CR⁴, wherein at least two of A are N;
each R⁴ is independently hydrogen, halogen, —CN, —NO₂, —OH, —OR^{a}, —SH, —SR^{a}, —NR^{c}R^{d}, C₁-C₆alkyl, C₁-C₆haloalkyl, C₁-C₆hydroxyalkyl, C₁-C₆aminoalkyl;
B is N or CR⁵;
E is N or CR⁷;
F is N or CR⁸;
R⁵, R⁷, and R⁸ are each independently hydrogen, halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, or C₁-C₆aminoalkyl; wherein each of the alkyl, haloalkyl, alkene, alkyne, and hydroxyalkyl, and aminoalkyl is optionally substituted with 1 to 4 substituents independently selected from R^{e};
R⁹ is hydrogen, C₁-C₆alkyl, C₁-C₆haloalkyl, C₃-C₆cycloalkyl, or 4 to 6-membered heterocycloalkyl, wherein each alkyl, haloalkyl, cycloalkyl and heterocycloalkyl is independently substituted with 1 to 4 substituents selected from R^{e};
each R¹⁰ is independently halogen, —CN, —NO₂, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₃-C₆cycloalkyl, or 4 to 6-membered heterocycloalkyl, wherein each of the alkyl, haloalkyl, hydroxyalkyl, aminoalkyl, cycloalkyl and heterocycloalkyl is optionally substituted with 1 to 4-substituents independently selected from R^{e}; or
two R¹⁰ are taken together to form an oxo;
each Rₐ is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R;
each R^{e} is independently halogen, oxo, —CN, —OH, —S(═O)CH₃, —S(═O)₂CH₃, —CH₂S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6-membered heteroaryl; and
p is 8;
q is 0, 1, 2, or 3.

In some cases, X is —CH₂—, —O—, —S—, or —NR³—; and Z is CH. In some cases, X is bond and Z is N. In some cases, each A is independently N or CH, wherein two of A are N. In some cases,

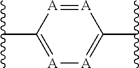

is

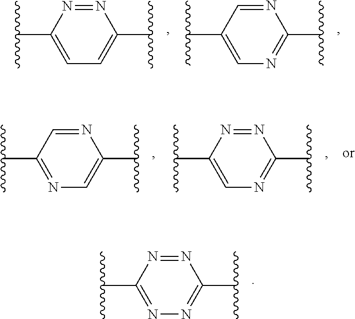

In some cases,

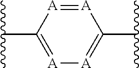

is

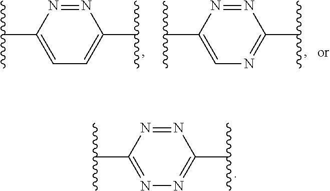

In some cases,

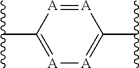

is

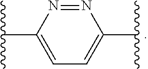 [SMILES: *c1ccc(*)nn1]

In some cases,

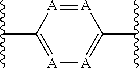

is

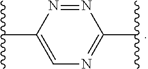 [SMILES: *c1cnc(*)nn1]

In some cases, compound has the structure of Formula (III′), or a pharmaceutically acceptable salt or solvate thereof:

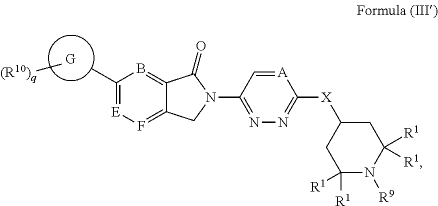

wherein,
ring G is a 5 to 10-membered heteroaryl;
each R¹ is independently hydrogen, halogen, —CN, —NO₂, —OH, —OR^{a}, —SH, —SR^{a}, —NR^{c}R^{d}, C₁-C₆alkyl, C₁-C₆haloalkyl, C₁-C₆hydroxyalkyl, or C₁-C₆aminoalkyl, wherein each of the alkyl, haloalkyl, hydroxyalkyl, or aminoalkyl is optionally substituted with 1 to 4 substituents independently selected from R^{e}; or
two R¹ are taken together to form a bond, C₁-C₃alkylene, or C₁-C₃heteroalkylene, wherein the alkylene or heteroalkylene is optionally substituted with 1 to 4 substituents independently selected from R^{e}; or
two R¹ are taken together to form a C₃-C₆cycloalkyl or 4 to 6-membered heterocycloalkyl, each of which is optionally substituted with 1 to 4 substituents independently selected from R;
X is —CH₂—, —O—, —S—, or —NR³—;
R³ is hydrogen, C₁-C₃alkyl, C₁-C₃hydroxyalkyl, C₁-C₃haloalkyl, or C₃-C₆ cycloalkyl;
A is N or CH;
B is N or CR⁵;
E is N or CR⁷;
F is N or CR⁸;
R⁵, R⁷, and R⁸ are each independently hydrogen, halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, or C₁-C₆aminoalkyl; wherein each of the alkyl, alkene, alkyne, haloalkyl, and hydroxyalkyl, and aminoalkyl is optionally substituted with 1 to 4 substituents independently selected from R^{e};
R⁹ is hydrogen, C₁-C₆alkyl, C₁-C₆haloalkyl, C₃-C₆cycloalkyl, or 4 to 6-membered heterocycloalkyl, wherein each alkyl, haloalkyl, cycloalkyl and heterocycloalkyl is independently substituted with 1 to 4 substituents selected from R^{e};
each R¹⁰ is independently halogen, —CN, —NO₂, —OH, —OR, —NR^{c}R^{d}, C₁-C₆alkyl, C₁-C₆haloalkyl, C₁-C₆hydroxyalkyl, C₁-C₆aminoalkyl, C₁-C₆heteroalkyl, C₃-C₆cycloalkyl, or 4 to 6-membered heterocycloalkyl, wherein each of the alkyl, haloalkyl, hydroxyalkyl, aminoalkyl, cycloalkyl and heterocycloalkyl is optionally substituted with 1 to 4-substituents independently selected from R^{e}; or
two R¹⁰ are taken together to form an oxo;
each Rₐ is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{e} is independently halogen, oxo, —CN, —OH, —S(═O)CH₃, —S(═O)₂CH₃, —CH₂S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6-membered heterocycloalkyl; and
q is 0, 1, 2, or 3.

In some cases, two R¹ are taken together to form a C₁-C₃alkylene or C₁-C₃heteroalkylene, wherein the alkylene or heteroalkylene is optionally substituted with 1 to 4 substituents independently selected from R^{e}. In some cases, R¹ are taken together to form a C₁-C₃alkylene.

In some cases, the compound of Formula (III′) has the structure of Formula (IIIa′), or a pharmaceutically acceptable salt or solvate thereof:

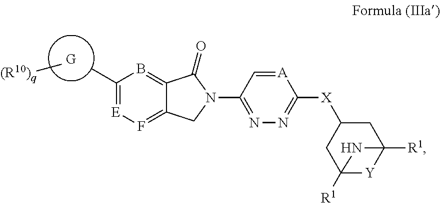

wherein Y is C₁-C₃alkylene. In some cases, Y is —CH₂CH₂—.

In some cases, the compound of Formula (III′) has the structure of Formula (IIIb′), or a pharmaceutically acceptable salt or solvate thereof:

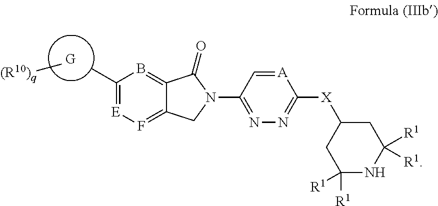

In some cases, two R¹ are taken together to form a C₃-C₆cycloalkyl. In some cases, two R¹ are taken together to form a cyclopropyl. In some cases, each R^{b} is independently hydrogen, halogen, C₁-C₆alkyl, or C₁-C₆haloalkyl, wherein each of the alkyl, haloalkyl, hydroxyalkyl, or aminoalkyl is optionally substituted with 1 to 4 substituents independently selected from R^{e}. In some cases, each R¹ is independently hydrogen, halogen, or C₁-C₆alkyl. In some cases, each R^{b} is independently hydrogen or methyl. In some cases, X is —O— or —NR³—. In some cases, X is —O—. In some cases, X is —NR³—. In some cases, R³ is hydrogen, C₁-C₃alkyl or C₃-C₆ cycloalkyl. In some cases, R³ is hydrogen, methyl, ethyl, or cyclopropyl. In some cases, R³ is hydrogen or methyl.

In some cases, compound has the structure of Formula (IV′), or a pharmaceutically acceptable salt or solvate thereof:

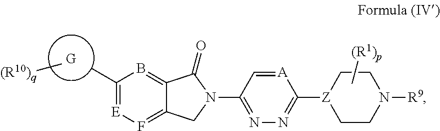

wherein,
ring G is a 5 to 10-membered heteroaryl;
each R¹ is independently hydrogen, halogen, —CN, —NO₂, —OH, —OR^{a}, —SH, —SR^{a}, —NR^{c}R^{d}, C₁-C₆alkyl, C₁-C₆haloalkyl, C₁-C₆hydroxyalkyl, or C₁-C₆aminoalkyl, wherein each of the alkyl, haloalkyl, hydroxyalkyl, or aminoalkyl is optionally substituted with 1 to 4 substituents independently selected from R^{e}; or
two R¹ are taken together to form an oxo; or
two R¹ are taken together to form a bond, C₁-C₃alkylene, or C₁-C₃heteroalkylene, wherein the alkylene or heteroalkylene is optionally substituted with 1 to 4 substituents independently selected from R^{e}; or
two R¹ are taken together to form a C₃-C₆cycloalkyl or 4 to 6-membered heterocycloalkyl, each of which is optionally substituted with 1 to 4 substituents independently selected from R^{e};
Z is CH or N;
A is N or CH;
B is N or CR⁵;
E is N or CR⁷;
F is N or CR⁸;
R⁵, R⁷, and R⁸ are each independently hydrogen, halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, or C₁-C₆aminoalkyl; wherein each of the alkyl, alkene, alkyne, haloalkyl, and hydroxyalkyl, and aminoalkyl is optionally substituted with 1 to 4 substituents independently selected from R^{e};
R⁹ is hydrogen, C₁-C₆alkyl, C₁-C₆haloalkyl, C₃-C₆cycloalkyl, or 4 to 6-membered heterocycloalkyl, wherein each alkyl, haloalkyl, cycloalkyl and heterocycloalkyl is independently substituted with 1 to 4 substituents selected from R^{e};
each R¹⁰ is independently halogen, —CN, —NO₂, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₃-C₆cycloalkyl, or 4 to 6-membered heterocycloalkyl, wherein each of the alkyl, haloalkyl, hydroxyalkyl, aminoalkyl, cycloalkyl and heterocycloalkyl is optionally substituted with 1 to 4-substituents independently selected from R^{e}; or
two R¹⁰ are taken together to form an oxo;
each Rₐ is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{e} is independently halogen, oxo, —CN, —OH, —S(═O)CH₃, —S(═O)₂CH₃, —CH₂S(═O)₂CH₃, —S(═O)₂NH₂, —S(═O)₂NHCH₃, —S(═O)₂N(CH₃)₂, —C(═O)NH₂, —C(═O)NHCH₃, —C(═O)N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6-membered heterocycloalkyl;
p is 8; and
q is 0, 1, 2, or 3.

In some cases, Z is CH. In some cases, Z is N. In some cases, two R¹ are taken together to form a C₁-C₃alkylene or C₁-C₃heteroalkylene, wherein the alkylene or heteroalkylene is optionally substituted with 1 to 4 substituents independently selected from R^{c}. In some cases, two R¹ are taken together to form a C₁-C₃alkylene. In some cases, two R¹ are taken together to form a C₃-C₆cycloalkyl. In some cases, each R^{b} is independently hydrogen, halogen, C₁-C₆alkyl, or C₁-C₆haloalkyl, wherein each of the alkyl, haloalkyl, or hydroxyalkyl is optionally substituted with 1 to 4 substituents independently selected from R^{e}. In some cases, each R^{b} is independently hydrogen, halogen, or C₁-C₆alkyl. In some cases, each R¹ is independently hydrogen or methyl. In some cases, A is N. In some cases, A is CH. In some cases, B is CR⁵. In some cases, B is N. In some cases, R⁵ is hydrogen, halogen, —CN, —NO₂, —OH, —OR^{a}, C₁-C₆alkyl, C₁-C₆haloalkyl, C₁-C₆hydroxyalkyl, or C₁-C₆aminoalkyl; wherein each of the alkyl, haloalkyl, hydroxyalkyl, and aminoalkyl is optionally substituted with 1 to 4 substituents independently selected from R^{e}. In some cases, R⁵ is hydrogen, halogen, C₁-C₆alkyl, or C₁-C₆haloalkyl. In some cases, B is CH. In some cases, F is CR⁸. In some cases, F is N. In some cases, R¹ is hydrogen, halogen, —CN, —NO₂, —OH, —OR^{a}, C₁-C₆alkyl, C₁-C₆haloalkyl, C₁-C₆hydroxyalkyl, or C₁-C₆aminoalkyl; wherein each of the alkyl, haloalkyl, hydroxyalkyl, and aminoalkyl is optionally substituted with 1 to 4 substituents independently selected from R^{e}. In some cases, R¹ is hydrogen, halogen, C₁-C₆alkyl, or C₁-C₆haloalkyl. In some cases, E is CR⁷. In some cases, E is N. In some cases, R⁷ is hydrogen, halogen, —CN, —NO₂, —OH, —OR, C₁-C₆alkyl, C₁-C₆haloalkyl, C₁-C₆hydroxyalkyl, or C₁-C₆aminoalkyl; wherein each of the alkyl, haloalkyl, hydroxyalkyl, and aminoalkyl is optionally substituted with 1 to 4 substituents independently selected from R^{e}. In some cases, R⁷ is hydrogen, halogen, C₁-C₆alkyl, or C₁-C₆haloalkyl. In some cases, E is CH. In some cases, ring G a 5 to 6-membered heteroaryl optional substituted with 1 or 4 substituents selected from R^{e}. In some cases, ring G is a 5-membered heteroaryl optional substituted with 1 or 2 substituents selected from R^{e}. In some cases, ring G is pyrrole, imidazole, triazole, tetrazole, thiophene, oxazole, thiazole, or thiadiazole. In some cases, ring G is:

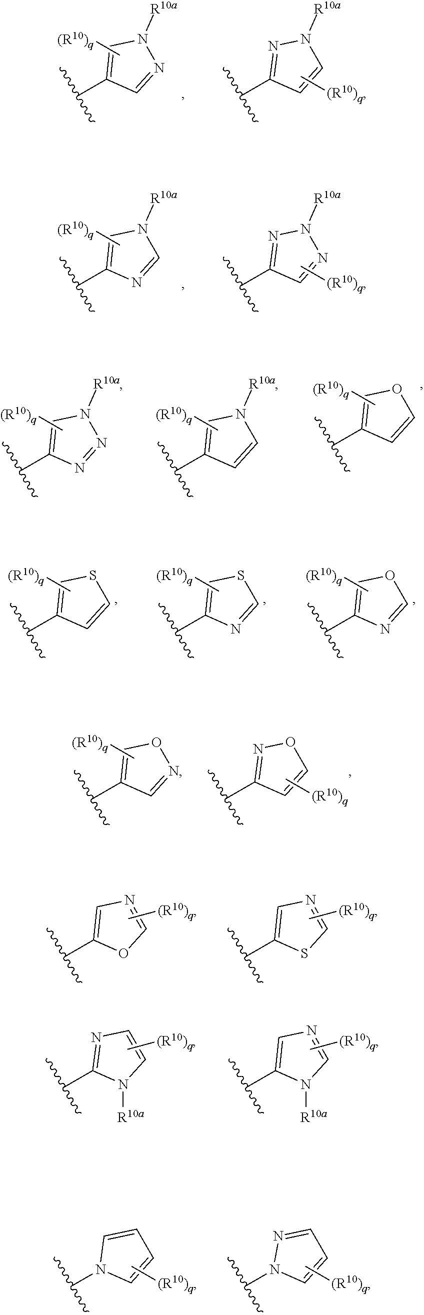

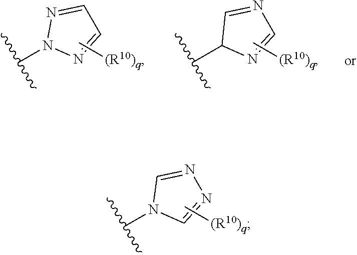

wherein,
each R¹⁰ is independently halogen, —CN, —NO₂, —OH, —OR^{a}, —NR^{c}R^{d}, C₁-C₆alkyl, C₁-C₆haloalkyl, C₁-C₆hydroxyalkyl, C₁-C₆aminoalkyl, C₃-C₆cycloalkyl, or 4 to 6-membered heterocycloalkyl wherein each of the alkyl, haloalkyl, hydroxyalkyl, aminoalkyl, cycloalkyl and heterocycloalkyl is optionally substituted with 1 to 4-substituents independently selected from R^{e};
each R^{10a} is independently hydrogen, C₁-C₆alkyl, C₁-C₆haloalkyl, C₃-C₆cycloalkyl, or 4 to 6-membered heterocycloalkyl, wherein each of the alkyl, haloalkyl, cycloalkyl and heterocycloalkyl is optionally substituted with 1 to 4-substituents independently selected from R^{e}; and
q is 0, 1, 2, or 3.

In some cases, each R¹⁰ is independently halogen, —CN, —NO₂, —OH, —OR^{a}, C₁-C₆alkyl, C₁-C₆haloalkyl, C₃-C₆cycloalkyl, or 4 to 6-membered heterocycloalkyl;
each R^{10a} is independently hydrogen, C₁-C₆alkyl, C₁-C₆haloalkyl, C₃-C₆cycloalkyl, or 4 to 6-membered heterocycloalkyl; and
q is 0, 1, or 2.

In some cases, each R¹⁰ is independently —F, —C₁, methyl, ethyl, —OCH₃, —CF₃, —CHF₂, or —CH₂F.

In some cases, each R^{10a} is independently C₁-C₆alkyl which is optionally substituted with 1 to 4-substituents independently selected from halogen, —OH, —CH₂S(═O)₂CH₃, C₁-C₆alkyl, C₁-C₆alkoxy, C₁-C₆haloalkyl, and C₁-C₆hydroxyalkyl.

In some cases, each R^{10′} is independently hydrogen, methyl, ethyl, isopropyl, tert-butyl, —CD₃, —CF₃, CHF₂, CH₂F, —CH₂CF₃, —CH₂SO₂CH₃, cyclopropyl, oxetanyl,

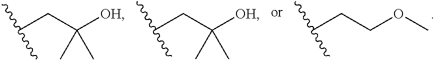

In some cases,

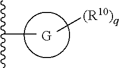

is

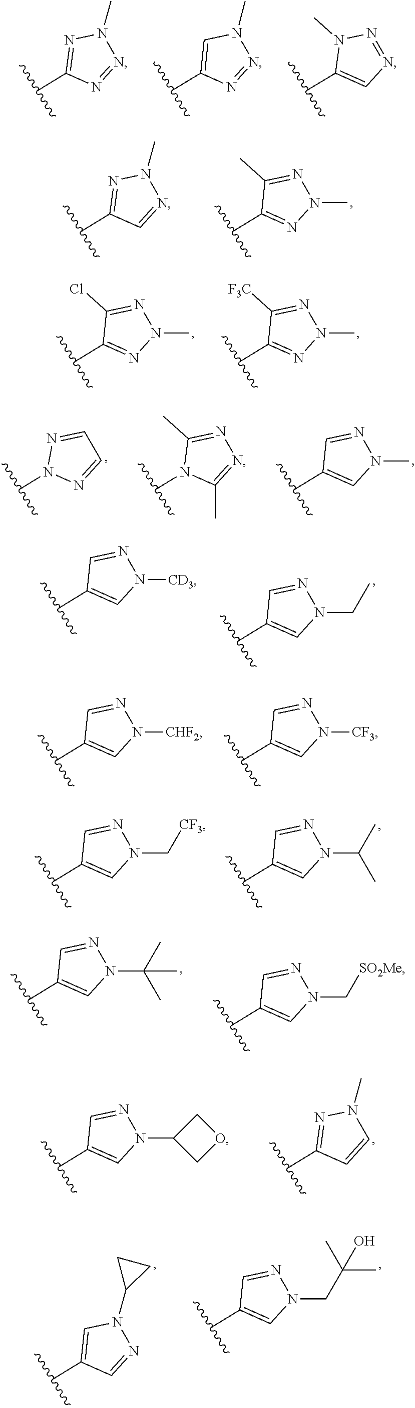

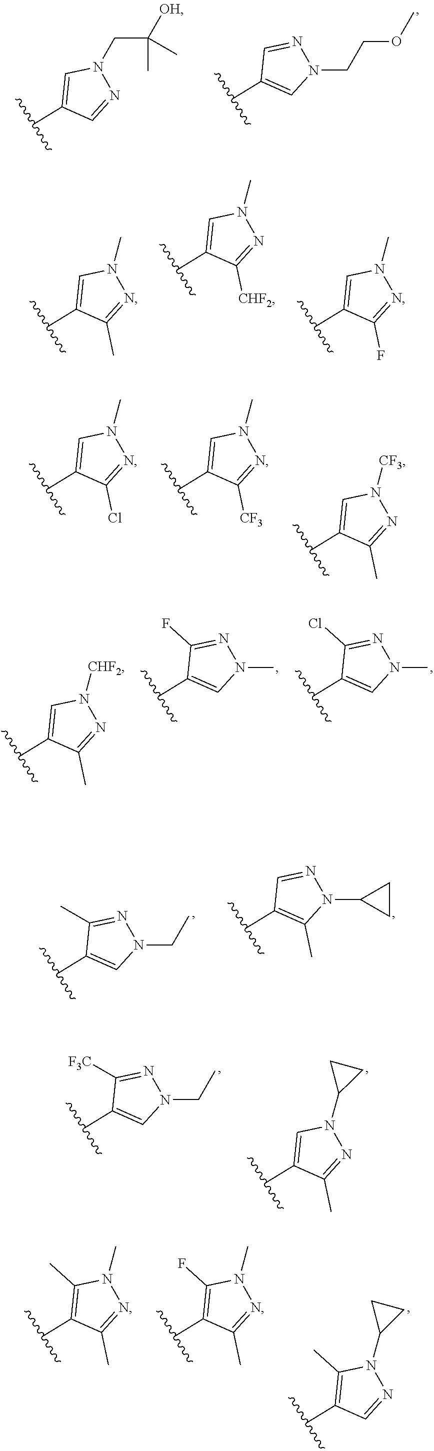

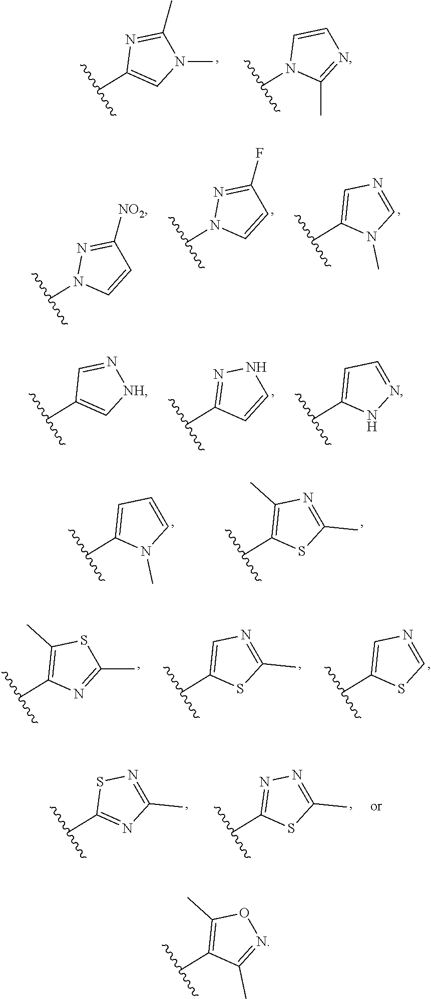

In some cases, is

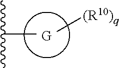

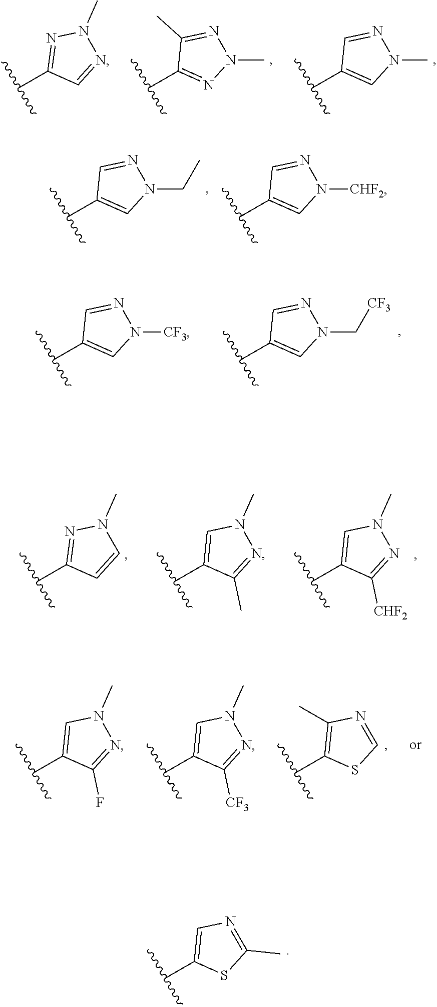

In some cases, ring G is a 6-membered heteroaryl optional substituted with 1 to 4 substituent selected from R^{e}. In some cases, ring G is pyridine, pyrimidine, or triazine. In some cases, ring G is:

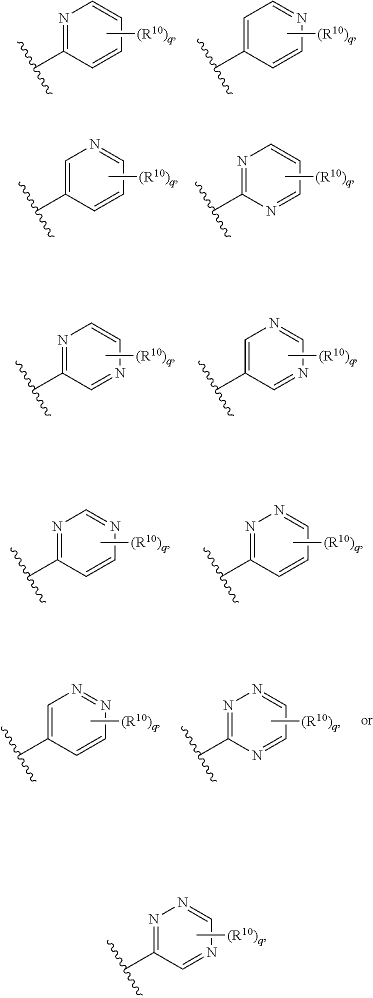

wherein,
each R¹⁰ is independently halogen, —CN, —NO₂, —OH, —OR^{a}, —NR^{c}R^{d}, C₁-C₆alkyl, C₁-C₆haloalkyl, C₁-C₆hydroxyalkyl, C₁-C₆aminoalkyl, C₃-C₆cycloalkyl, or 4 to 6-membered heterocycloalkyl, wherein each of the alkyl, haloalkyl, hydroxyalkyl, aminoalkyl, cycloalkyl and heterocycloalkyl is optionally substituted with 1 to 4-substituents independently selected from R^{e}; and
q is 0, 1, 2, or 3.

In some cases, each R¹⁰ is independently —OCH₃ or —N(CH₃)₂; and q is 0, 1, or 2.

In some cases, is

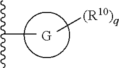

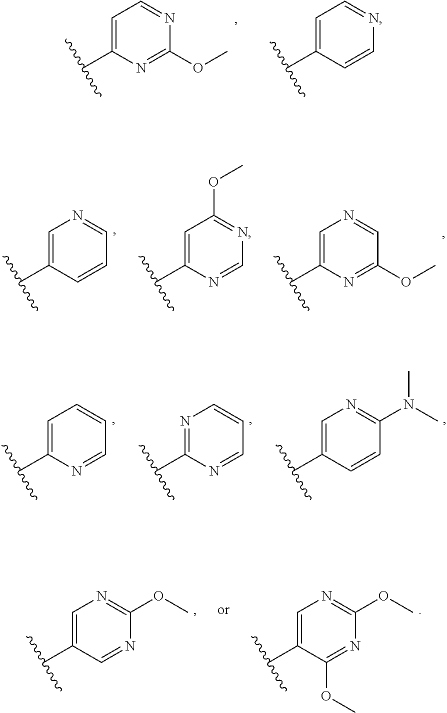

In some cases, ring G is a 9 or 10-membered bicyclic heteroaryl comprising 1 to 3 heteroatoms selected from N, O, and S; and optional substituted with 1 to 4 substituents selected from R^{e}.

In some cases, ring G is a 9-membered bicyclic heteroaryl comprising 1 to 3 heteroatoms selected from N; and optional substituted with 1 to 4 substituents selected from R^{e}.

In some cases, each Rₐ is independently C₁-C₆alkyl, C₁-C₆haloalkyl, or C₁-C₆hydroxyalkyl, wherein each of the alkyl, haloalkyl, and hydroxyalkyl is optionally substituted with 1 to 4 substituents independently selected from R^{e};
each R^{b} is independently hydrogen, C₁-C₆alkyl, C₁-C₆haloalkyl, or C₁-C₆hydroxyalkyl, wherein each of the alkyl, haloalkyl, and hydroxyalkyl is optionally substituted with 1 to 4 substituents independently selected from R^{e}; and
R^{c} and R^{d} are each independently hydrogen, C₁-C₆alkyl, or C₁-C₆haloalkyl, wherein each of the alkyl, and haloalkyl is optionally substituted with 1 to 4 substituents independently selected from R^{e}.

In some cases, each Rₐ is independently C₁-C₆alkyl;
each R^{b} is independently hydrogen or C₁-C₆alkyl; and
R^{c} and R^{d} are each independently hydrogen or C₁-C₆alkyl.

In some cases, each Rₐ is independently methyl, ethyl, isopropyl, or tert-butyl;
each R^{b} is independently hydrogen, methyl or ethyl; and
R^{c} and R^{d} are each independently hydrogen, methyl, or ethyl.

In some cases, each R^{e} is independently halogen, —CN, —OH, —S(═O)CH₃, —S(═O)₂CH₃, —CH₂S(═O)₂CH₃, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₃-C₆cycloalkyl, or 4 to 6-membered heterocycloalkyl. In some cases, each R is independently —F, —C₁, —OH, —CH₂S(═O)₂CH₃, methyl, ethyl, isopropyl, tertbutyl, —OCH₃, cyclopropyl, cyclobutyl, cyclopentyl, cyclohexyl, or azetidine, oxytane, piperidine, piperazine, or morpholine. In some cases, each R is independently —F, —OH, —CF₃, —CHF₂, —OCH₃, or methyl.

In some cases, the compound is selected from a compound of Table 3A, Table 3B, Table 3C, Table 4, or Table 5, or a pharmaceutically acceptable salt or solvate thereof.

In some cases, the compound is selected from the group consisting of:

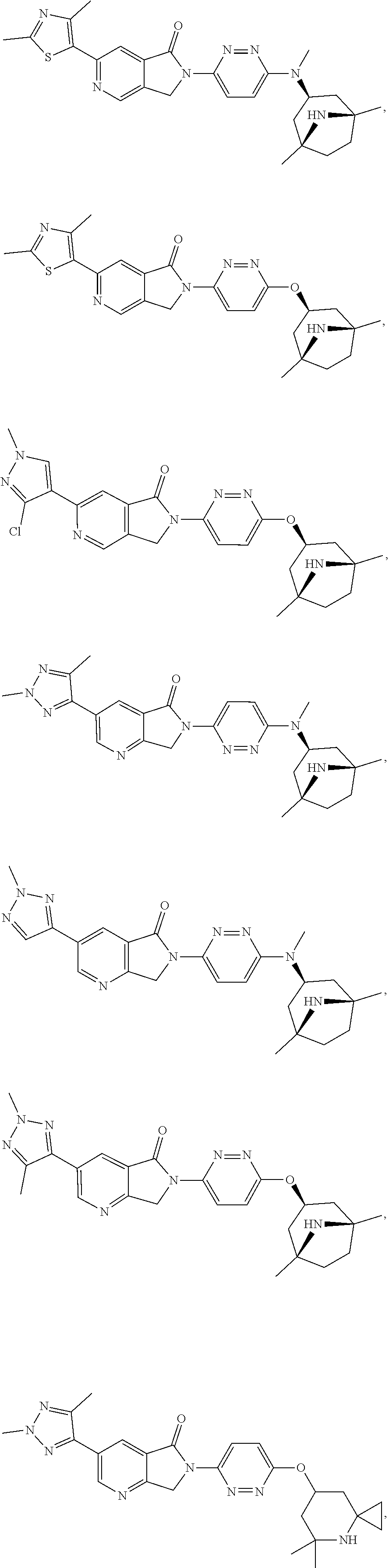

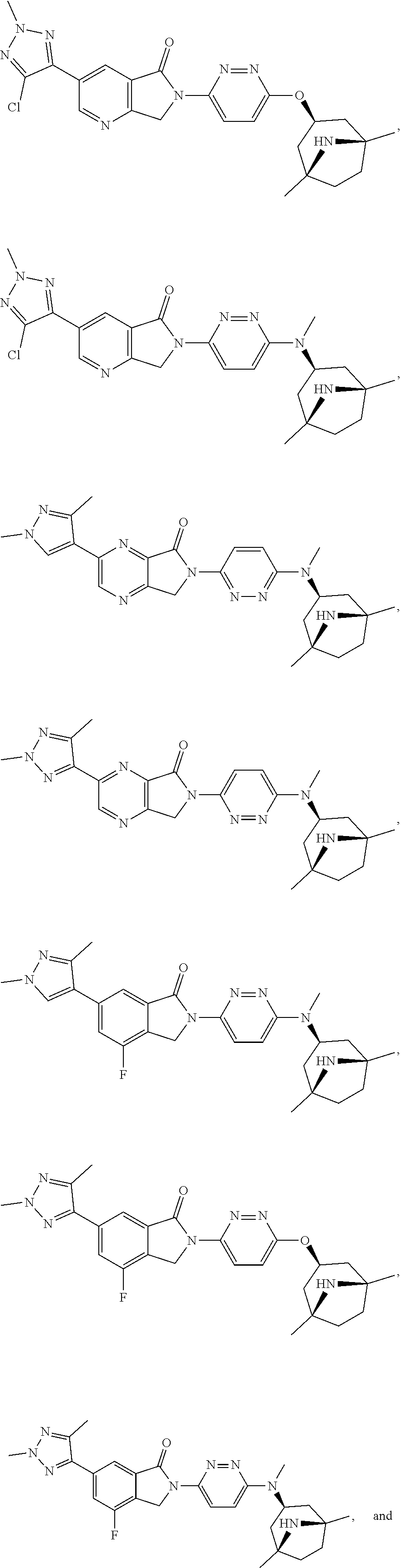

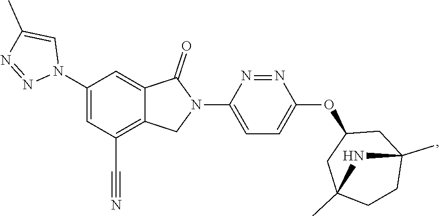 [SMILES: Cc1cn(-c2cc(C#N)c3c(c2)C(=O)N(c2ccc(O[C@H]4C[C@]5(C)CC[C@](C)(C4)N5)nn2)C3)nn1]

or a pharmaceutically acceptable salt or solvate thereof.

In some cases, the compound is provided in a pharmaceutical composition comprising a compound of any one of the preceding, or a pharmaceutically acceptable salt or solvate thereof; and a pharmaceutically acceptable excipient.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1A and FIG. 1B illustrate progression of nucleotide repeat expansion during a subject's lifespan. FIG. 1A illustrates manifestation of a nucleotide repeat expansion disease or condition triggered by the accumulation of the nucleotide repeat expansion. FIG. 1B illustrates the nucleotide repeat expansion occurring at an allelic level.

FIG. 2 illustrates a flowchart of nucleotide repeat expansion mechanism. Without wishing to be bound by theory, nucleotide repeat expansion forms a hairpin in the DNA (top), where the presence of the hairpin recruits MutS homolog (MSH) complexes for repairing the hairpin (middle). The repair of the hairpin creates a gap in the genome, where the gap is filled with additional nucleotide repeats, leading to nucleotide repeat expansion (bottom).

FIG. 3 illustrates compounds capable of downregulating MSH3 mRNA expression, where the decreased expression of MSH3 can inhibit repair and the subsequent nucleotide repeat expansion of a target gene.

FIG. 4 illustrates the presence of an exemplary poison exon in a gene. Without wishing to be bound by theory, under physiological conditions, Exon n and Exon n+1 are spliced together in an mRNA transcript to encode a functional protein. The poison is typically spliced out of the mRNA transcript. When the poison exon is spliced in to the mRNA transcript, a Premature Termination Codon (PTC) is introduced into the mRNA transcript. The mRNA transcript comprising the poison exon, instead of being translated into a functional protein, is degraded by endogenous cellular mechanisms such as nonsense-mediated mRNA decay (NMD).

FIG. 5 illustrates a non-limiting illustration of an exemplary agent of the disclosure in a complex with the pre-mRNA and the U1 snRNA. Without wishing to be bound by theory, the agent may enhance binding and/or stabilization of the U1 snRNA to the pre-mRNA comprising the poison exon. FIG. 5 discloses SEQ ID NO: 12.

FIG. 6 illustrates screening and identifying of poison exons in the MSH3 gene that can be modulated by agents of the disclosure.

FIG. 7 illustrates examples of agents of the disclosure that modulate the splicing of Exon 21A into the MSH3 mRNA transcript.

FIG. 8 illustrates splicing and mRNA expression changes for targets regulated by an agent of the disclosure. Compound 1 and CHX increased poison exon expression (Exon 21A) and decreased total RNA (e.g., due to NMD).

FIG. 9 illustrates splicing and mRNA expression changes for targets regulated by an agent of the disclosure. Compound 1 and CHX increased poison exon expression (Exon 14A_1 or Exon 14A_2) and decreased total RNA (e.g., due to NMD).

FIGS. 10A-10C illustrate an exemplary system for screening and validating an agent of the disclosure.

FIG. 11A illustrates MSH3 total expression (assay spanning exons 13/14) in cells treated with an exemplary compound of the disclosure. FIG. 11B illustrates MSH3 PE5 expression (assay spanning exons 13/14A) in cells treated with an exemplary compound of the disclosure.

FIG. 12A illustrates MSH3 total expression (assay spanning exons 20/21) in cells treated with an exemplary compound of the disclosure. FIG. 12B illustrates MSH3 PE4 expression (assay spanning exons 20/21A) in cells treated with an exemplary compound of the disclosure. FIG. 12C illustrates MSH3 protein expression in cells treated with an exemplary compound of the disclosure.

FIG. 13 illustrates modulation of splicing of MSH3 poison exon 14A in cynomolgus monkey and human cells treated with an exemplary compound of the disclosure, as determined by RT-PCR.

### DETAILED DESCRIPTION

Unless defined otherwise, all technical and scientific terms used herein have the same meaning as is commonly understood by one of skill in the art to which this invention belongs. All patents and publications referred to herein are incorporated by reference.

Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to ═O.

“Carboxyl” refers to —COOH.

“Cyano” refers to —CN.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁-3alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system can contain only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Carbocycle” refers to a saturated, unsaturated, or aromatic rings in which each atom of the ring is carbon. Carbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. An aromatic ring, e.g., phenyl, may be fused to a saturated or unsaturated ring, e.g., cyclohexane, cyclopentane, or cyclohexene. Any combination of saturated, unsaturated, and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Unless stated otherwise specifically in the specification, a carbocycle may be optionally substituted.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom), spiro, or bridged ring systems. In some embodiments, the cycloalkyl is fully saturated. Representative cycloalkyls include, but are not limited to, cycloalkyls having from three to fifteen carbon atoms (e.g., C₃-C₁₅ fully saturated cycloalkyl or C₃-C₁₅ cycloalkenyl), from three to ten carbon atoms (e.g., C₃-C₁₀ fully saturated cycloalkyl or C₃-C₁₀ cycloalkenyl), from three to eight carbon atoms (e.g., C₃-C₈ fully saturated cycloalkyl or C₃-C₈ cycloalkenyl), from three to six carbon atoms (e.g., C₃-C₆ fully saturated cycloalkyl or C₃-C₆ cycloalkenyl), from three to five carbon atoms (e.g., C₃-C₅ fully saturated cycloalkyl or C₃-C₅ cycloalkenyl), or three to four carbon atoms (e.g., C₃-C₄ fully saturated cycloalkyl or C₃-C₄ cycloalkenyl). In some embodiments, the cycloalkyl is a 3- to 10-membered fully saturated cycloalkyl or a 3- to 10-membered cycloalkenyl. In some embodiments, the cycloalkyl is a 3- to 6-membered fully saturated cycloalkyl or a 3- to 6-membered cycloalkenyl. In some embodiments, the cycloalkyl is a 5- to 6-membered fully saturated cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Cycloalkenyl” refers to an unsaturated non-aromatic monocyclic or polycyclic hydrocarbon radical consisting solely of carbon and hydrogen atoms, which includes fused or bridged ring systems, preferably having from three to twelve carbon atoms and comprising at least one double bond. In certain embodiments, a cycloalkenyl comprises three to ten carbon atoms. In other embodiments, a cycloalkenyl comprises five to seven carbon atoms. The cycloalkenyl may be attached to the rest of the molecule by a single bond. Examples of monocyclic cycloalkenyls includes, e.g., cyclopentenyl, cyclohexenyl, cycloheptenyl, and cyclooctenyl.

“Halo” or “halogen” refers to bromo, chloro, fluoro or iodo. In some embodiments, halogen is fluoro or chloro. In some embodiments, halogen is fluoro.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., C₁, Br, F, I, etc.). When an alkyl group is substituted with more than one halogen radicals, each halogen may be independently selected e.g., 1-chloro, 2-fluoroethane.

“Fluoroalkyl” refers to an alkyl radical, as defined above, that is substituted by one or more fluoro radicals, for example, trifluoromethyl, difluoromethyl, fluoromethyl, 2,2,2-trifluoroethyl, 1-fluoromethyl-2-fluoroethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (e.g., C₂-C₁₅ fully saturated heterocycloalkyl or C₂-C₁₅ heterocycloalkenyl), from two to ten carbon atoms (e.g., C₂-C₁₀ fully saturated heterocycloalkyl or C₂-C₁₀ heterocycloalkenyl), from two to eight carbon atoms (e.g., C₂—C₈ fully saturated heterocycloalkyl or C₂-C₈ heterocycloalkenyl), from two to seven carbon atoms (e.g., C₂-C₇ fully saturated heterocycloalkyl or C₂-C₇ heterocycloalkenyl), from two to six carbon atoms (e.g., C₂-C₆ fully saturated heterocycloalkyl or C₂-C₆ heterocycloalkenyl), from two to five carbon atoms (e.g., C₂-C₅ fully saturated heterocycloalkyl or C₂-C₅ heterocycloalkenyl), or two to four carbon atoms (e.g., C₂-C₄ fully saturated heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered fully saturated heterocycloalkyl. In some embodiments, the heterocycloalkyl is a 3- to 7-membered fully saturated heterocycloalkyl. In some embodiments, the heterocycloalkyl is a 3- to 6-membered fully saturated heterocycloalkyl. In some embodiments, the heterocycloalkyl is a 4- to 6-membered fully saturated heterocycloalkyl. In some embodiments, the heterocycloalkyl is a 5- to 6-membered fully saturated heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “substituted” refers to moieties having substituents replacing a hydrogen on one or more carbons or substitutable heteroatoms, e.g., NH, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched, and unbranched, carbocyclic, and heterocyclic, aromatic, and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

The term “one or more” when referring to an optional substituent means that the subject group is optionally substituted with one, two, three, or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition, or vehicle, such as a liquid or solid filler, diluent, excipient, solvent, or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

An “effective amount” or “therapeutically effective amount” refers to an amount of a compound administered to a mammalian subject, either as a single dose or as part of a series of doses, which is effective to produce a desired therapeutic effect.

The terms “treat,” “treating” or “treatment,” as used herein, include alleviating, abating, or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition.

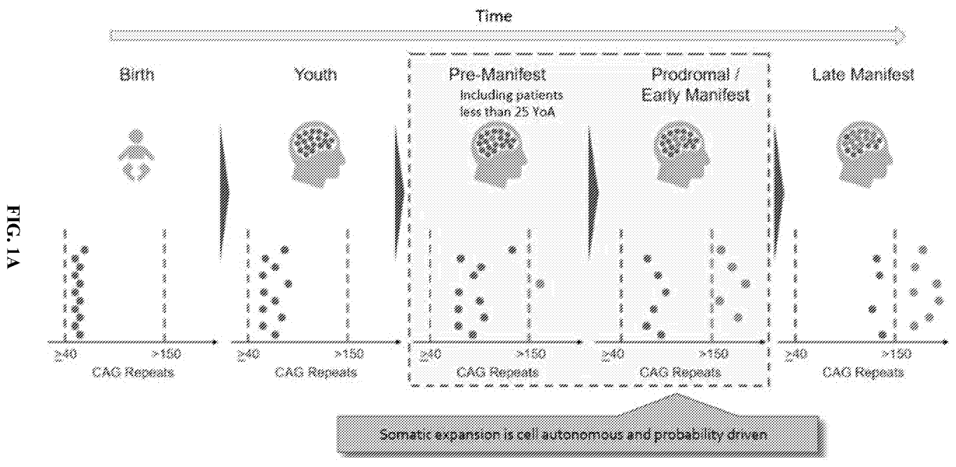

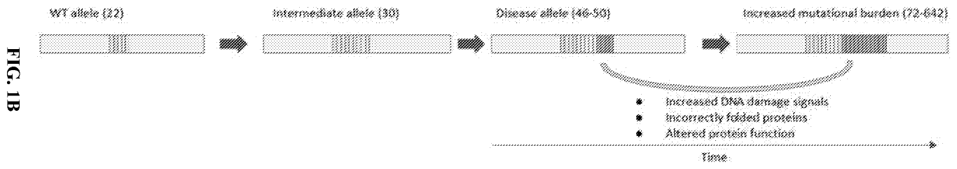

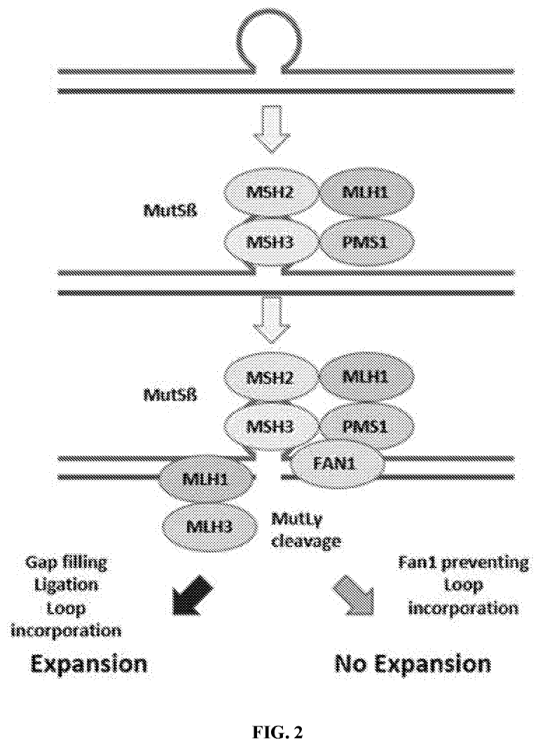

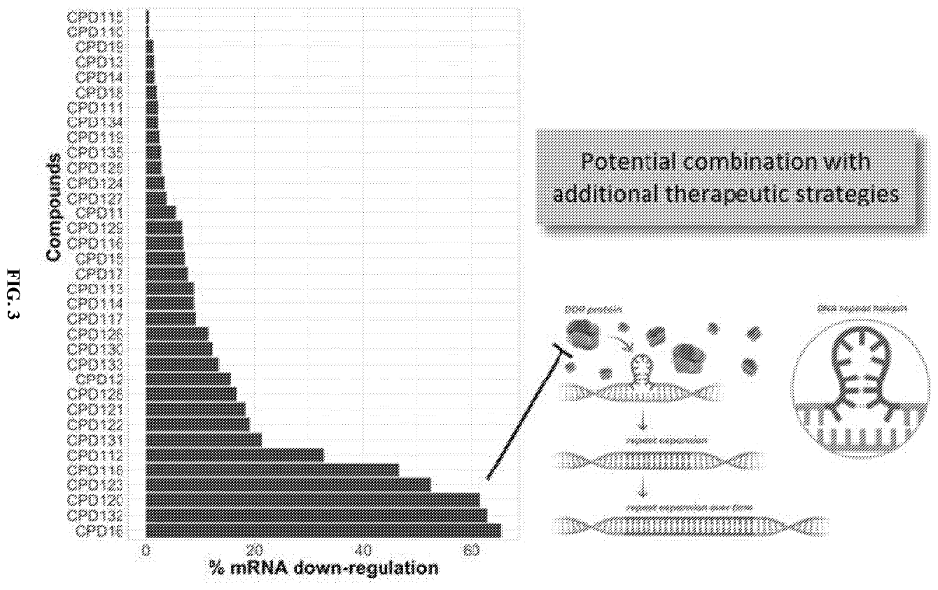

Diseases or conditions associated with nucleotide repeat expansion are prevalent. Nucleotide repeat expansion may be caused by a wild-type (WT) allele first having certain nucleotide repeats (e.g., CAG repeat for Huntington's disease). The presence of nucleotide repeats at low abundance may not manifest into pathology. However, as the nucleotide repeat accumulates (or expands), symptoms of the disease or condition begin to manifest. FIG. 1A and FIG. 1B illustrate this concept. Without wishing to be bound by theory, as illustrated by FIG. 2, nucleotide repeats are thought to lead to formation of a hairpin loop in the genome, where the hairpin loop serves as a substrate for repair complexes such as MutS homolog (MSH) complexes for repairing the hairpin loop. Upon repair of the hairpin loop, aberrant gap filling at the site of the repair creates longer nucleotide repeats or nucleotide repeat expansion. FIG. 2 depicts components of the MutS homolog (MSH) complex that may be targeted for modulating the repair of the nucleotide repeat. For example, by modulating and inhibiting expression of MSH3, the repair of the nucleotide repeat is inhibited or decreased, thus decreasing nucleotide repeat expansion.

Provided herein are compositions, methods, and systems for modulating splicing of MSH3 pre-mRNA such that the resulting MSH3 mRNA transcript includes at least one poison exon. The poison exon introduces a Premature Termination Codon (PTC) into the mRNA transcript, and leads to elimination of the MSH3 mRNA transcript containing the PTC(s) by, e.g., the nonsense-mediated mRNA decay (NMD) pathway. Elimination of the MSH3 mRNA transcript may lead to reduced expression of MSH3 protein. Reduction in the levels of MSH3 protein may lead to a reduction in the activity of the DNA mismatch repair mechanism associated with nucleotide repeat expansion, which, in turn, may lead to reduction of nucleotide repeat expansion of a target gene (e.g., HTT). The compositions, methods, and systems provided herein generally involve the use of agents (e.g., small molecules) that modulate the splicing of MSH3 pre-mRNA such that the resulting MSH3 mRNA transcript includes the poison exon(s). Without wishing to be bound by theory, the agent may enhance and/or stabilize binding of small nuclear RNA (e.g., U1 snRNA) to the pre-mRNA transcript, thereby causing splicing in of the poison exon into the MSH3 mRNA. Without wishing to be bound by theory, the agent may form a complex with the snRNA (e.g., U1 snRNA) and the pre-mRNA transcript. Further provided herein are compositions, methods, and systems for treating a disease or condition (e.g., using the agents provided herein). In some cases, the disease or condition is an expansion repeat disease or condition.

In some aspects, provided herein is a method for modulating splicing of MSH3 pre-mRNA, the method comprising contacting a cell with an agent which modulates splicing of MSH3 pre-mRNA such that an MSH3 mRNA transcript comprising a poison exon is produced, thereby modulating splicing of MSH3 pre-mRNA.

In another aspect, provided herein is a method for treating a disease or condition in a subject, the method comprising administering to the subject an agent which modulates splicing of MSH3 pre-mRNA such that an MSH3 mRNA transcript comprising a poison exon is produced in a cell of the subject, thereby treating the disease or condition. In some embodiments, the disease or condition described herein is associated with a gene comprising a nucleotide repeat expansion as described herein. Table 6 illustrates examples of diseases or conditions associated with a gene having a nucleotide repeat expansion that may be treated using the compositions, methods, and systems provided herein.

In another aspect, provided herein is a system comprising: (a) an MSH3 pre-mRNA; (b) a spliceosomal RNA; and (c) an agent which modulates splicing of the MSH3 pre-mRNA such that an MSH3 mRNA transcript comprising the poison exon is produced. In another aspect, provided herein are compositions comprising an agent for modulating splicing of MSH3 pre-mRNA such that an MSH3 mRNA transcript comprising a poison exon is produced.

In another aspect, a vessel is provided containing any system of the disclosure. The vessel may be selected from the group consisting of: a vial, a tube, a plate, and a dish. In another aspect, a cell is provided containing any system of the disclosure.

In another aspect, an mRNA transcript is provided comprising an MSH3 mRNA and at least one poison exon, as described herein. In some cases, the mRNA transcript is non-naturally occurring (e.g., does not exist in nature in the absence of the compositions, methods, and systems provided herein).

In some embodiments, the compositions, methods, and systems provided herein involve modulating splicing of MSH3 pre-mRNA such that the MSH3 mRNA transcript contains a poison exon. In some embodiments, the poison exon comprises a nucleic acid sequence that has at least about 70%, at least about 75%, at least about 80%, at least about 85%, at least about 90%, at least about 95%, or at least about 99% sequence identity to the nucleic acid sequence of SEQ ID NO: 1. In some embodiments, the poison exon consists of or comprises the nucleic acid sequence of SEQ ID NO: 1, or a portion thereof. In some embodiments, the poison exon consists of or comprises Exon 21A, or a portion thereof, of MSH3. In some embodiments, the poison exon is located at or within position chir5:80832901-80832994 of Human Genome Assembly hg38 of December 2013.

In some embodiments, the poison exon comprises a nucleic acid sequence that has at least about 7000, at least about 7500, at least about 80%, at least about 85%, at least about 90%, at least about 95%, or at least about 990 sequence identity to SEQ ID NO: 2 or SEQ ID NO: 3. In some embodiments, the poison exon consists of or comprises the nucleic acid sequence of SEQ ID NO: 2 or SEQ ID NO: 3, or a portion thereof. In some embodiments, the poison exon consists of or comprises Exon 14A_1, or a portion thereof, of MSH3. In some embodiments, the poison exon consists of or comprises Exon 14A_2, or a portion thereof, of MSH3. In some embodiments, the poison exon is located at or within position chr5:80763222-80763369 or position chr5:80763222-80767932 of Human Genome Assembly hg38 of December 2013. In some embodiments, the poison exon is selected from the group consisting of any one of the poison exons listed in Table 1. In some embodiments, the poison exon introduces a premature termination codon (PTC) into the MSH3 mRNA transcript. In some embodiments, the poison exon may comprise a splice donor site according to any one of the splice donor sites described in Table 2. In some embodiments, the poison exon may be located at any of the chromosomal locations described in Table 2. In some embodiments, the poison exon may comprise a PTC beginning at a chromosomal location as described in Table 2.

TABLE 1
MSH3 poison exons

| Poison Exon | Nucleic acid sequence (5′ to 3′) |
|---|---|
| Exon 21A | CATTTTCCTTGCAAAAGGCCCAGAGATTGTGATTTGAAGGGACAGAAGCCCACTC |
| | CATTTGCCTGAGAGACATCTAGAATATTATCTCAAAATA (SEQ ID NO: 1) |
| Exon 14A_1 | AGTTTTACTAGAATGATAAGTACTGCTTCTAAGAATACTTGAGGTGCCCAAGAGG |
| | ACTTACGTAACTGTTTTACATGCCTCAGCTAAATTTGAAAACAATGCACAAGCCAG |
| | AACTGGAAATACTGATCCTGGGGAGAAAGATATATGA (SEQ ID NO: 2) |
| Exon 14A_2 | AGTTTTACTAGAATGATAAGTACTGCTTCTAAGAATACTTGAGGTGCCCAAGAGG |
| | ACTTACGTAACTGTTTTACATGCCTCAGCTAAATTTGAAAACAATGCACAAGCCAG |
| | AACTGGAAATACTGATCCTGGGGAGAAAGATATATGAGTTGGTTATGGGGCCGGG |
| | GAGCCTATTGCATCATTATTGGGTACACCATGTTATTGTTTTGCTGGCAATAGCAG |
| | AAGCTGTGCTGATAGTCGCAAGTTTTATGGTAGACCTTTTTGAAGTTGCAATGTGG |
| | GTGCCAAACCATTTGTTGTTTTTATCCTTGGCTCAGAGCCTGAAAATTGATGCATA |
| | TTAACCTTCTTTAAACAACAGATCTCCAGGCACCTTGCCTTAACTACTTCAAAGGA |
| | TTCACTGACAGATGTCAGAGATTTGAAAACAGAAGCAAAGCTGAAAATCTTTCAG |
| | TGGCTACTCAAATGCACAAAGTCAAAGTGTCCATGGCATCAAGATGGCAGCATCA |
| | GTTCATGGGGCTGTGATTTCTATTCCACCTTCCTGCCTTTTTGTAAACTTTACTTTG |
| | ATGTTTGTATTGCACAGGGTTTGTGTATTTATAAAAGTTTCAAAACTCTAGTAAAG |
| | TCAGAATCTAATTCATTTAAAAAGTGGCCGGTATGCAGTGCAAGAATGACTTATTT |
| | TGGAATTTCAAAATATAGTTTACTAACGGAACTTATAACTCACTTGGAAGCCATTC |
| | CTTGTTGCTGTTTTAGTGTTTGTTTACACAAGAATGTACCTAAAGATTAGTTTGAA |
| | GGGTGAAGAGTAGAAATGATCTGGTAACAAACCAACTTCAGGAAAGCTGAATGCT |
| | TTAAACCAATGTTTTATAGATTAGTTGGCTGGCTCTGTGTACACACCATCAAACAT |
| | ATGTCTGTTCTTTATTATTAGATTACTCTAAATAAGCTTCTACTGTGTACACCACAT |
| | AGTGGGTGATGTCCTGGGTACAGATTGTGCCAATTATGTGAAATTCATAAAATGA |
| | GAAGGAACACTGGATGTTGTGGGAAGTGGTTAGCATAGGCTGTATGGTTTTGTGG |
| | AACGTGCCCTGGAATATGTACCAATAGGTGTTTTTATGGTGATAATGATAGAGGAT |
| | CTCAAGTGAGCTGATTCTGGTATAAAATAGACATTTCTTCTTCTTCTTTTTTTTTTT |
| | GAAACAAGATCTCACTGTGTCACCCATGCTGGAATGCAGTGGTGCGACCACAGCT |
| | CACTACAGCTTCAGCCTCCAGCCTTCTGAGTAGTTGGGACAACAGGCACGTGCCA |
| | CCATGCCCAGCTATTTTTTTTTTTTTTTTTGCATTTTTTTGTAGAGACAAGGTGTTGC |
| | CATGTTGCCCAGGCTGGTCTCCAGCTCCTGGGTTCAAGAGATCCTCCCACCTTGGA |
| | CTCCCAGTGTGTTGGGATTACGGGTGTGAGCCACTGTGCTTAGCCTAAAATAAACA |
| | TTTCTTTTGATGGCAAAATGTTAAATTCAAATCCTGTAAATAGTGCTAGTCACAGG |
| | AACCCATAATAACTTTGCTCAAGAACTGTTTTGTAAGAAATTCTTAAGTAACTGCC |
| | GAGGTTGGTGAATACATTTAAAGGATAATCGCACTTAGCAAATGTTGAGGAGAGT |
| | GTTTTCAGAATTTCCTAGGTGAAAATCTTTTTTTAGCTAAGCAGGAATAAGCCCAC |
| | AGCTGCATGTTCAGTGTGGCACTTAAGCTGGGGTCCTTCCCATCCTGTGTCCAGTG |
| | AGGCCCTGCCACCATACTTGTGGGTCTGTGCTCAAGCTCTCCCAGTGAGACAGCTG |
| | CTGCCAGAAGTAGGGCTGAACTCCTGGAGAGTGACTTCAGGAGCAGCCACTTACC |
| | CATCCTCAGCTTTCTTGTAGCTTCTTTTGAGTTCTTTTGCTTAAGAAACTTGAGAAC |
| | AACCTGCTGCCTCTGGCTTTCAACATGATTGTAAAGCACTACTGGTTTTCACCTTG |
| | CTTCTGTTGCTGACTTAACTTTACATTGTGTCTCAGCTTGGAAAATTAATCATTTTC |
| | TTGGGATTACAATAATCATAAGAGGTTAATATTCAAAATTATATTGAAAATGTTTG |
| | TGTTTGTTTCTTTACTTTTGTTAAAAGGTATCATTAATTGACTAGTGGCCAAAAAGC |
| | TTCCAGGTTTCACATTTATTTAAACAGTCCAGCTTCCTGTATAGGGTGGGGAGGGG |
| | GTCTTTCTGGAGAAAAGTTGCCTCATGATACACTTAGAATTCAACATTTGCTCAGT |
| | TGGACCTAACTTCTTATTTTTTTTGCATAATTCACCCTTTGTGGTTAGTAATTACCT |
| | TTTTGAATACGTTCTTAGCCTTAATCTATTTCATATATACTATCTATTATGTGCCTT |
| | ATTACTTTACCCCTAAATTGTTTTCTCATCTCAAGACAGTTCTTTTCAGCCAACATT |
| | TTGTCAGTTTTATTTTCCTGAGCTGTCTTCTAGAGCCTTCTAACTTGATCCAGTCTG |
| | GACTGGTTGTCCCCATATCTGTTGTATAGTACTATCTTAGATTCCCTTTACTTCCCT |
| | TGAGTTGAATCTTTTATTTTCTATATTCCATGTCTTCCTCTTCGTTTATTTCTTCATT |
| | TTGGTGGAGTATATATCCTCTAGAACCTTCCTGAGAAAGCACGTTGGAAGTAAATC |
| | TTTTGAAACATTCATGTCTGAAAGTGGTTTTTTTGTTGTTGTTATTATTGTTACTAC |
| | CTTCACATTTGATTGGTAGTTTGGATTGGTATAAAATTCTAGGTTTTAGATGACTTC |
| | ATTATCTTTACTTCCTCTTCTGTTGAATTTTTCATTTCTACTATTATGCTTTAATTTC |
| | CAAGAATTTTTGTTTTCTTGTCCTCCAAATGTTTTTTCTTTTTAAAAGTAGCATCTT |
| | GTTTCATGGTTGTAGTATCTTCTGTTTCATGGAAGATATTATTAATATTTTTAGAAG |
| | TTCCCTAACAGGGCAGAATCCATATTGGTACACAAGTCTTTTTGGATTCTTGATGT |
| | AAATTGTGTCTCCGTTTTCCTTAAGGACAGTTTAAAATTCAGATACCTTGAGTCTTT |
| | TAAGTCAATTACTGTTTATTTGTTTACTTTATAGCTTCTAACATATTATTGCTGTTG |
| | TCTTCTTTTCTAGTGTGTTTTTTTCCTTTTTTTTTTTTTTTTTTTTTTTTGAGACGGAA |
| | TCTTGCTCTGTTGCCCAGGCAGGAGTGCAGTGGCACGATCTCGGCTCACTGCAGTC |
| | TCCGCCTTCCAGGCTCAAGCGATTCTCCTGCCTCGCCACCTGAGTCACTGGGATCA |
| | CAGGCGAGCGCCACCACACCCAGCTAATTTTTGTATTTTTAGTAGAGATGGGGTTT |
| | CACCATGTTGGTAGGGCTGGTCTCGAACTCCTGACCTTGTGATCTGCCCGCCTCAG |
| | CCTCCCAAAGTGCTGGGATTACAGGCGTGAGTCACTGCGTCTGGCTGGTTTTATGC |
| | CGTTTTAAACAATCTCTTAACTGTCGTTTCAAGATGGAGAGAAATTAGATGCATGT |
| | GTTTAACTCACTGTTTACCAGAAGCTATGATATAAAAATTATTCATGTTCATGTTA |
| | AAAAAAAAACTACAGTAGTATGCAGGGGTATGCAGTAAAGGGTAAAAAGTCTCC |
| | CTCTGCCACCCTTCCATACCTACTACCCAGAGGTAACCAGAGTAAGTCACATGTGT |
| | TACCTCCTAAAATTTTTATTGATATTTAGATTTTTTATTTATATAGAATAATGTATA |
| | TCATTTAAAGAATACAAAGAAAGTGTTACCATGTAAATACATAAAAATATATACA |
| | TATATGGTATATGATATAAACATACTGTATATATTCAATGTATGTTGAAATTATGG |
| | AGTTCTATACTGATGCAAACAGATCTTAATTTTCTTTTAGCTGTTTCGTAGTACTAC |
| | ACTGCATGAGTTTTTCCCAATAAGATCCCAATTGATGGACATTTATTTCTTATTCAT |
| | CTTTTTAAATTATAAGCAGCGCTACTATGAATATTCAGACACACATATTTTTCACT |
| | ATTTGTTAGTCTATTTTTAGATAAATTATTTAAAGTGGTGCTTATTCAAAGGGTTAT |
| | GTGCACTTTTTTATTACGAAGACATGCCAAGTTGTTTTTCAAAAAGGTTAGTCCTC |
| | TGCTAAGAGTATATAAAAGTGTCTTTTTTTTCTTTTACATCCTGTTACATGGTTATT |
| | GTGAAGCTTTTAAATCTTTGGCGTTCCGATAAGTGAAAAATGCTATCCTATATTTC |
| | TAATTTGCTTGTCTTTAATTATATATGAAGATAATCGTATACATACTTGCTGTTCTC |
| | TTTCTATGAGCTGCATATTCAGTTTGTTTAGCCATTGTTCTTTGGATTATTCATCTTT |
| | TTCTTGTCAATTTGTAAGAGCCTATTATGTTTGGGAGAATTTTGCCTTGTGTCTGTT |
| | ATGCCTTACAGATATTATCCTTCACATTTTTTAACTTGTTTAACCTATTTTTGCTAT |
| | ATGGTTTATGTTATTTTAAAAATGAAAAAATACTAAACAAGTATTGGCTTTGTTTT |
| | CCTTGCCTAAAATTATTTCCTTTATACCTTTAAAATATTACATAGTATTTAACCTCA |
| | TTTAGAAAATATCAGTGATATTTTATTTCAACTAACTAGTTAATAGAAAATTTAAA |
| | CTTTCCTCAATTCTGATTCTAACTTTTAAAAGTCACTAATTAAACTTCATTTTCATG |
| | TATCTTATGCTATTTCATAAAAAATATTTCTATTTTCAG (SEQ ID NO: 3) |

TABLE 2
MSH3 poison exon splice donor sites

| Poison exon region | Donor site | PTC |
|---|---|---|
| chr5: 80848448-80848612 | CAGGTAAAG | 80848474 |
| chr5: 80719484-80719535 | TATGTAAGT | 80725463 |
| chr5: 80830637-80830740 | TTCGTAAGT | 80830684 |
| chr5: 80875163-80875265 | CAGGTACTC | 80875180 |
| chr5: 80762038-80762127 | AAGGTGAGT | 80762081 |
| chr5: 80752160-80752256 | GAGGTAAGG | 80752192 |
| chr5: 80697162-80697289 | AAGGTGCCT | 80697194 |
| chr5: 80832901-80832994 | ATAGTAAGA | 80832936 |
| chr5: 80792227-80792455 | AAGGTAAAC | 80792235 |
| chr5: 80790565-80790680 | AAGGTATAA | 80790573 |
| chr5: 80746749-80746917 | ATGGTTTGG | 80746811 |
| chr5: 80869483-80869661 | AAGGTATTC | 80869513 |
| chr5: 80739646-80739775 | GAGGTAAAG | 80739666 |
| chr5: 80763222-80763369 | ATGAGTTGGT (SEQ ID NO: 4) | 80763262 |
| chr5: 80763222-80763409 | TTGGGTACAC (SEQ ID NO: 5) | 80763262 |
| chr5: 80763222-80767315 | AAAGGTTAGT (SEQ ID NO: 6) | 80763262 |
| chr5: 80814725-80832994 | AATAGTAAGA (SEQ ID NO: 7) | 80814796 |
| chr5: 80814725-80836261 | TTGAGTAAGT (SEQ ID NO: 8) | 80814796 |
| chr5: 80814725-80848612 | GCAGGTAAAG (SEQ ID NO: 9) | 80814796 |
| chr5: 80814725-80851699 | CTTGGTATGT (SEQ ID NO: 10) | 80814796 |
| chr5: 80814725-80852834 | AAAGGTACAT (SEQ ID NO: 11) | 80814796 |

In some embodiments, the poison exon can decrease expression of the MSH3 mRNA transcript in a cell. In some embodiments, the poison exon is a cryptic exon that is not normally spliced into the MSH3 mRNA. In some embodiments, the poison exon is an exon that is not spliced into the MSH3 mRNA, in the absence of an agent that modulates splicing of the MSH3 pre-mRNA, as disclosed herein. In some embodiments, the MSH3 mRNA transcript comprising the poison exon that is produced by the compositions, methods, and systems provided herein, is a non-naturally occurring MSH3 mRNA transcript. In other words, the MSH3 mRNA comprising the poison exon that is produced by the compositions, methods, and systems provided herein does not ordinarily occur in nature, absent intervention with the agent provided herein. In some embodiments, the poison exon is spliced into the MSH3 mRNA transcript when contacted with an agent described herein. In some embodiments, the poison exon, when spliced into the MSH3 mRNA transcript, decreases expression of the MSH3 mRNA transcript. In some embodiments, the poison exon introduces a PTC into the MSH3 mRNA transcript. In some embodiments, the PTC directs the MSH3 mRNA transcript for degradation, thereby decreasing expression of the MSH3 mRNA transcript. In some cases, the MSH3 mRNA transcript is degraded by nonsense-mediated mRNA decay (NMD). In some embodiments, MSH3 expression is decreased by at least 0.1 fold, at least 0.2 fold, at least 0.5 fold, at least 1.0 fold, at least 2.0 fold, at least 5.0 fold, at least 10.0 fold, or at least about 100.0 fold, compared to a level of MSH3 in absence of the agent.

In some embodiments, the compositions, methods, and systems provided herein involve the use of an agent that induces splicing of a poison exon into MSH3 mRNA transcript. Without wishing to be bound by theory, in some embodiments, the agent may form a complex with the MSH3 pre-mRNA (e.g., at the poison exon) and a small nuclear RNA (snRNA) (e.g., U1 snRNA). Without wishing to be bound by theory, in some embodiments, the agent may enhance and/or stabilize binding of the snRNA to the MSH3 pre-mRNA (e.g., at the poison exon), such that a poison exon is spliced into the MSH3 mRNA. In some embodiments, the snRNA is part of the spliceosome. In some embodiments, the snRNA is complexed with small nuclear ribonucleoproteins (snRNP). In some embodiments, the snRNA is a spliceosomal RNA. In some embodiments, the snRNA is a U1 spliceosomal RNA. In some embodiments, the presence of the poison exon in the MSH3 mRNA transcript leads to elimination or reduction of MSH3 mRNA transcript such that expression of the MSH3 protein is reduced. Generally, the poison exon introduces a premature termination codon (PTC) into the MSH3 mRNA transcript. In some embodiments, the poison exon induces degradation of the MSH3 mRNA transcript by an endogenous mechanism such as nonsense-mediated mRNA decay (NMD). In some cases, the MSH3 pre-mRNA comprising the poison exon, the agent, and the snRNA are in a complex. In some embodiments, the agent causes splicing of the poison exon into the MSH3 mRNA transcript (i.e., in the absence of the agent, the poison exon is spliced out of the MSH3 mRNA transcript). In some embodiments, the agent is a compound, such as a small molecule. In some embodiments, the agent is any compound disclosed herein. In some embodiments, the agent is for use in a method of treatment of the human or animal body by therapy. In some embodiments, the agent is for use in a method of treatment of a nucleotide expansion repeat disease or condition.

In some embodiments, the spliceosomal RNA comprises a small nuclear RNA (snRNA). In some embodiments, the snRNA is selected from the group consisting of: U1 snRNA, U2 snRNA, U4 snRNA, U5 snRNA, U6 snRNA, and any combination thereof. In some embodiments, the snRNA comprises U1 snRNA. In some embodiments, the snRNA comprises U2 snRNA. In some embodiments, the snRNA comprises U4 snRNA. In some embodiments, the snRNA comprises U5 snRNA. In some embodiments, the snRNA comprises U6 snRNA. In some embodiments, the MSH3 pre-mRNA and the spliceosomal RNA are partially complementary. In some embodiments, the MSH3 pre-mRNA and the U1 snRNA are partially complementary. In some embodiments, the MSH3 pre-mRNA and the U2 snRNA are partially complementary. In some embodiments, the MSH3 pre-mRNA and the U4 snRNA are partially complementary. In some embodiments, the MSH3 pre-mRNA and the U5 snRNA are partially complementary. In some embodiments, the MSH3 pre-mRNA and the U6 snRNA are partially complementary.

In some embodiments, the MSH3 pre-mRNA and the spliceosomal RNA are partially complementary, where the complexing between the MSH3 pre-mRNA and the spliceosomal RNA is caused by an agent described herein. In some embodiments, the agent described herein induces the complexing between the MSH3 pre-mRNA and the spliceosomal RNA. In some embodiments, the agent, upon complexing with the MSH3 pre-mRNA and the spliceosomal RNA, induces splicing in of the poison exon into the MSH3 mRNA.

In some embodiments, the poison exon and the spliceosomal RNA are partially complementary. In some embodiments, the poison exon and the U1 snRNA are partially complementary. In some embodiments, the poison exon and the U2 snRNA are partially complementary. In some embodiments, the poison exon and the U4 snRNA are partially complementary. In some embodiments, the poison exon and the U5 snRNA are partially complementary. In some embodiments, the poison exon and the U6 snRNA are partially complementary.

In some embodiments, the poison exon and the spliceosomal RNA are partially complementary, where the complexing between the poison exon and the spliceosomal RNA is caused by an agent described herein. In some embodiments, the agent described herein induces the complexing between the poison exon and the spliceosomal RNA. In some embodiments, the agent, upon complexing with the poison exon and the spliceosomal RNA, induces splicing in of the poison exon into the MSH3 mRNA.

In some embodiments, the agent is selected from the group consisting of: a small molecule, a polypeptide, an oligosaccharide, a polysaccharide, an oligonucleotide, an aptamer, a ribonucleoprotein (RNP), an enzyme, a nuclease (e.g., a Cas nuclease), and any combination thereof. In some embodiments, the agent comprises a gene editing tool, (e.g., CRISPR/Cas, TALENs, zinc finger nucleases, homing endonucleases or meganucleases, etc.) for targeting and inclusion of a poison exon into MSH3.

In particular embodiments, the agent is a small molecule. In some embodiments, the agent is any molecule or compound described herein.

In some embodiments, the compound is represented by Formula (I″), or a pharmaceutically acceptable salt or solvate thereof:

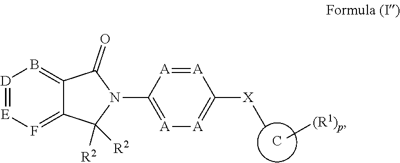

wherein,
ring C is 4 to 10-membered heterocycloalkyl or 4 to 10-membered heterocycloalkenylene;
each R¹ is independently hydrogen, halogen, —CN, —NO₂, —OH, —OR^{a}, —SH, —SR^{a}, —NR^{c}R^{d}, C₁-C₆alkyl, C₁-C₆haloalkyl, C₁-C₆hydroxyalkyl, or C₁-C₆aminoalkyl, wherein each of the alkyl, haloalkyl, hydroxyalkyl, or aminoalkyl is optionally substituted with 1 to 4 substituents independently selected from R^{e}; or
two R¹ are taken together to form an oxo; or
two R¹ are taken together to form a bond, C₁-C₃alkylene, or C₁-C₃heteroalkylene, wherein the alkylene or heteroalkylene is optionally substituted with 1 to 4 substituents independently selected from R^{e}; or
two R¹ are taken together to form a C₃-C₆cycloalkyl or 4 to 6-membered heterocycloalkyl, each of which is optionally substituted with 1 to 4 substituents independently selected from R;
each R² is independently hydrogen, halogen, —OH, C₁-C₆alkyl, C₁-C₆haloalkyl, C₁-C₆hydroxyalkyl, or C₁-C₆aminoalkyl; or
X is absent, —CH₂—, —O—, —OCH₂—, or —NR³—;
R³ is hydrogen, C₁-C₃alkyl, C₁-C₃hydroxyalkyl, C₁-C₃haloalkyl, or C₃-C₆ cycloalkyl;
each A is independently N or CR⁴, wherein at least two of A are N;
each R⁴ is independently hydrogen, halogen, —CN, —NO₂, —OH, —OR^{a}, —SH, —SR^{a}, —NR^{c}R^{d}, C₁-C₆alkyl, C₁-C₆haloalkyl, C₁-C₆hydroxyalkyl, or C₁-C₆aminoalkyl;
or R³ and one of R⁴ are taken together to form a 5 or 6-membered heterocycloalkylene or 5 or 6-membered heteroarylene;
B is N or CR⁵;
D is N or CR⁶;
E is N or CR⁷;
F is N or CR⁸;
R⁵, R⁶, R⁷ or R⁸ are independently hydrogen, halogen, —CN, —NO₂, —OH, —OR, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, C₁-C₆aminoalkyl, C₃-C₆cycloalkyl, 4 to 6 membered heterocycloalkyl, 5 to 10-membered heteroaryl, or O—(C₁-C₆alkylene)-5 to 10-membered heteroaryl; wherein each of the alkyl, alkene, alkyne, haloalkyl, hydroxyalkyl, aminoalkyl, cycloalkyl, heterocycloalkyl, or heteroaryl is optionally substituted with 1 to 4 substituents independently selected from R*;
wherein at least one of R⁵, R⁶, R⁷ or R⁸ is C₂-C₆alkene, C₃-C₆cycloalkyl, 4 to 6 membered heterocycloalkyl, 5 to 10-membered heteroaryl, or —O—(C₁-C₆alkylene)-5 to 10-membered heteroaryl, wherein the alkene, cycloalkyl, heterocycloalkyl and heteroaryl is optionally substituted with 1 to 4 substituents selected from R^{e};
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{e} is independently halogen, oxo, —CN, —OH, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(O)NH₂, —C(O)NHCH₃, —C(O)N(CH₃)₂, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6-membered heterocycloalkyl; and
p is an integer from 1-9.

In some embodiments, the compound is represented by Formula (I′), or a pharmaceutically acceptable salt or solvate thereof:

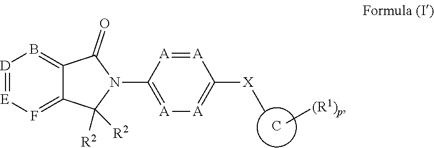

wherein,
ring C is 4 to 10-membered heterocycloalkyl or 4 to 10-membered heterocycloalkenylene;
each R¹ is independently hydrogen, halogen, —CN, —NO₂, —OH, —OR^{a}, —SH, —SR^{a}, —NR^{c}R^{d}, C₁-C₆alkyl, C₁-C₆haloalkyl, C₁-C₆hydroxyalkyl, or C₁-C₆aminoalkyl, wherein each of the alkyl, haloalkyl, hydroxyalkyl, or aminoalkyl is optionally substituted with 1 to 4 substituents independently selected from R^{e}; or
two R¹ are taken together to form an oxo; or
two R¹ are taken together to form a bond, C₁-C₃alkylene or C₁-C₃heteroalkylene, wherein the alkylene or heteroalkylene is optionally substituted with 1 to 4 substituents independently selected from R^{e}; or
two R¹ are taken together to form a C₃-C₆cycloalkyl or 4 to 6-membered heterocycloalkyl, each of which is optionally substituted with 1 to 4 substituents independently selected from R^{e};
each R² is independently hydrogen, halogen, —OH, C₁-C₆alkyl, C₁-C₆haloalkyl, C₁-C₆hydroxyalkyl, or C₁-C₆aminoalkyl; or
two R² are taken together to form an oxo;
X is absent, —CH₂—, —O—, —S—, or —NR³—; wherein R³ is hydrogen, C₁-C₃alkyl, C₁-C₃hydroxyalkyl, C₁-C₃haloalkyl, or C₃-C₆ cycloalkyl;
each A is independently N or CR⁴, wherein at least two of A are N;
each R⁴ is independently hydrogen, halogen, —CN, —NO₂, —OH, —OR^{a}, —SH, —SR^{a}, —NR^{c}R^{d}, C₁-C₆alkyl, C₁-C₆haloalkyl, C₁-C₆hydroxyalkyl, or C₁-C₆aminoalkyl;
B is N or CR₅;
D is N or CR⁶;
E is N or CR⁷;
F is N or CR⁸;
R⁵ and R⁸ are each independently hydrogen, halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, or C₁-C₆aminoalkyl; wherein each of the alkyl, haloalkyl, and hydroxyalkyl, and aminoalkyl is optionally substituted with 1 to 4 substituents independently selected from R^{e};
one of R⁶ or R⁷ is a 5 to 10-membered heteroaryl optionally substituted with 1 to 4 substituents independently selected from R¹⁰; and the other of R⁶ or R⁷ is hydrogen, halogen, —CN, —NO₂, —OH, —OR, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, C₁-C₆aminoalkyl, C₃-C₆cycloalkyl, or 4 to 6 membered heterocycloalkyl; wherein each of the alkyl, alkene, alkyne, haloalkyl, hydroxyalkyl, aminoalkyl, cycloalkyl, and heterocycloalkyl is optionally substituted with 1 to 4 substituents independently selected from R; wherein R⁷ is not pyridyl substituted with two methoxy groups;
each R¹⁰ is independently halogen, —CN, —NO₂, —OH, —OR, —NR^{c}R^{d}, C₁-C₆alkyl, C₁-C₆haloalkyl, C₁-C₆hydroxyalkyl, C₁-C₆aminoalkyl, C₁-C₆heteroalkyl, C₃-C₆cycloalkyl, or 4 to 6-membered heterocycloalkyl, wherein each of the alkyl, haloalkyl, hydroxyalkyl, heteroalkyl, cycloalkyl and heterocycloalkyl is optionally substituted with 1 to 4-substituents independently selected from R^{e}; or
two R¹⁰ are taken together to form an oxo;
each Rₐ is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R;
each R^{e} is independently halogen, oxo, —CN, —OH, —S(═O)CH₃, —S(═O)₂CH₃, —CH₂S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(O)NH₂, —C(O)NHCH₃, —C(O)N(CH₃)₂, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6-membered heterocycloalkyl; and
p is an integer from 1-9.

In some embodiments of Formula (I″), (I′), or (I), each R² is independently hydrogen, halogen, —OH, C₁-C₆alkyl, C₁-C₆haloalkyl, C₁-C₆hydroxyalkyl, or C₁-C₆aminoalkyl. In some embodiments of Formula (I″), (I′), or (I), each R² is independently halogen. In some embodiments of Formula (I″), (I′), or (I), each R² is independently C₁-C₆alkyl, C₁-C₆haloalkyl, C₁-C₆hydroxyalkyl, or C₁-C₆aminoalkyl. In some embodiments of Formula (I″), (I′), or (I), each R² is independently C₁-C₆alkyl. In some embodiments of Formula (I″), (I′), or (I), each R² is independently C₁-C₆haloalkyl. In some embodiments of Formula (I″), (I′), or (I), each R² is independently C₁-C₆hydroxyalkyl. In some embodiments of Formula (I″), (I′), or (I), each R² is independently C₁-C₆aminoalkyl. In some embodiments of Formula (I″), (I′), or (I), each R² is independently —OH. In some embodiments of Formula (I″), (I′), or (I), each R² is independently hydrogen.

In some embodiments of Formula (I″), (I′), or (I), two R² together with the atom to which they are attached form an oxo.

In some embodiments of Formula (I″), (I′), or (I), D is CR⁶. In some embodiments of Formula (I″), (I′), or (I), D is N.

In some embodiments of Formula (I″) or (I′), R⁶ is a 5 to 10-membered heteroaryl optionally substituted with 1 to 4 substituents selected from R¹⁰; and R⁷ is hydrogen, halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, C₁-C₆aminoalkyl, C₃-C₆cycloalkyl, or 4 to 6 membered heterocycloalkyl; wherein each of the alkyl, alkene, alkyne, haloalkyl, hydroxyalkyl, aminoalkyl, cycloalkyl, heterocycloalkyl, and heteroaryl is optionally substituted with 1 to 4 substituents independently selected from R*. In some embodiments of Formula (I″), (I′), or (I), R⁶ is a 5 to 8-membered heteroaryl optionally substituted with 1 to 4 substituents selected from R¹⁰; and R⁷ is hydrogen, halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, C₁-C₆aminoalkyl, C₃-C₆cycloalkyl, or 4 to 6 membered heterocycloalkyl; wherein each of the alkyl, alkene, alkyne, haloalkyl, hydroxyalkyl, aminoalkyl, cycloalkyl, heterocycloalkyl, and heteroaryl is optionally substituted with 1 to 4 substituents independently selected from R^{e}. In some embodiments of Formula (I″), (I′), or (I), R⁶ is a 5 to 8-membered heteroaryl optionally substituted with 1 to 4 substituents independently selected from R¹⁰; and R⁷ is hydrogen, halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₃-C₆cycloalkyl, or 4 to 6 membered heterocycloalkyl. In some embodiments of Formula (I″), (I′), or (I), R⁶ is a 5 to 8-membered heteroaryl optionally substituted with 1 to 4 substituents independently selected from R¹⁰; and R⁷ is hydrogen, halogen, —OH, —OR^{a}, C₁-C₆alkyl, or C₁-C₆haloalkyl. In some embodiments of Formula (I″), (I′), or (I), R⁶ is a 5 to 8-membered heteroaryl optionally substituted with 1 to 4 substituents independently selected from R¹⁰; and R⁷ is hydrogen, halogen, or C₁-C₆alkyl. In some embodiments of Formula (I″), (I′), or (I), R⁶ is a 5 to 8-membered heteroaryl optionally substituted with 1 to 4 substituents independently selected from R¹⁰; and R⁷ is hydrogen.

In some embodiments of Formula (I″) or (I′), R⁷ is a 5 to 10-membered heteroaryl optionally substituted with 1 to 4 substituents selected from R¹⁰; wherein R⁷ is not pyridyl substituted with two methoxy groups; and R⁶ is hydrogen, halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, C₁-C₆aminoalkyl, C₃-C₆cycloalkyl, or 4 to 6 membered heterocycloalkyl; wherein each of the alkyl, alkene, alkyne, haloalkyl, hydroxyalkyl, aminoalkyl, cycloalkyl, heterocycloalkyl, and heteroaryl is optionally substituted with 1 to 4 substituents independently selected from R^{e}. In some embodiments of Formula (I″), (I′), or (I), R⁷ is a 5 to 8-membered heteroaryl optionally substituted with 1 to 4 substituents selected from R¹⁰; wherein R⁷ is not pyridyl substituted with two methoxy groups; and R⁶ is hydrogen, halogen, —CN, —NO₂, —OH, —OR, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, C₁-C₆aminoalkyl, C₃-C₆cycloalkyl, or 4 to 6 membered heterocycloalkyl; wherein each of the alkyl, alkene, alkyne, haloalkyl, hydroxyalkyl, aminoalkyl, cycloalkyl, heterocycloalkyl, and heteroaryl is optionally substituted with 1 to 4 substituents independently selected from R^{e}. In some embodiments of Formula (I″), (I′), or (I), R⁷ is a 5 to 8-membered heteroaryl optionally substituted with 1 to 4 substituents independently selected from R¹⁰; and R⁶ is hydrogen, halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₃-C₆cycloalkyl, or 4 to 6 membered heterocycloalkyl. In some embodiments of Formula (I″), (I′), or (I), R⁷ is a 5 to 8-membered heteroaryl optionally substituted with 1 to 4 substituents independently selected from R¹⁰; and R⁶ is hydrogen, halogen, —OH, —OR^{a}, C₁-C₆alkyl, or C₁-C₆haloalkyl. In some embodiments of Formula (I″), (I′), or (I), R⁷ is a 5 to 8-membered heteroaryl optionally substituted with 1 to 4 substituents independently selected from R¹⁰; and R⁶ is hydrogen, halogen, or C₁-C₆alkyl. In some embodiments of Formula (I″), (I′), or (I), R⁷ is a 5 to 8-membered heteroaryl optionally substituted with 1 to 4 substituents independently selected from R¹⁰; and R⁶ is hydrogen.

In some embodiments of Formula (I″), (I′), or (I), ring C is a 4 to 10-membered heterocycloalkenylene. In some embodiments of Formula (I″), (I′), or (I), ring C is a 4 to 8-membered heterocycloalkenylene. In some embodiments of Formula (I″), (I′), or (I), ring C is a 4 to 6-membered heterocycloalkenylene. In some embodiments of Formula (I″), (I′), or (I), ring C is a 6-membered heterocycloalkenylene. In some embodiments of Formula (I″), (I′), or (I), ring C is a 7-membered heterocycloalkenylene. In some embodiments of Formula (I″), (I′), or (I), ring C is a 8-membered heterocycloalkenylene.

In some embodiments of Formula (I″), (I′), or (I), ring C is a is a 4 to 10-membered heterocycloalkyl. In some embodiments of Formula (I″), (I′), or (I), ring C is a is a 4 to 8-membered heterocycloalkyl. In some embodiments of Formula (I″), (I′), or (I), ring C is a is a 4 to 6-membered heterocycloalkyl. In some embodiments of Formula (I″), (I′), or (I), ring C is a 4-membered heterocycloalkyl. In some embodiments of Formula (I″), (I′), or (I), ring C is a 5-membered heterocycloalkyl. In some embodiments of Formula (I″), (I′), or (I), ring C is a 6-membered heterocycloalkyl. In some embodiments of Formula (I″), (I′), or (I), ring C is a 7-membered heterocycloalkyl. In some embodiments of Formula (I″), (I′), or (I), ring C is a 8-membered heterocycloalkyl.

In some embodiments of Formula (I″), (I′), or (I), ring C is piperidine, piperazine, or morpholine. In some embodiments of Formula (I″), (I′), or (I), ring C is piperidine. In some embodiments of Formula (I″), (I′), or (I), ring C is piperazine. In some embodiments of Formula (I″), (I′), or (I), ring C is morpholine.

In some embodiments of Formula (I″), (I′), or (I),

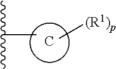

is

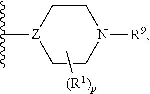

wherein,
Z is CH or N;
each R¹ is independently hydrogen, halogen, —CN, —NO₂, —OH, —OR^{a}, —SH, —SR^{a}, —NR^{c}R^{d}, C₁-C₆alkyl, C₁-C₆haloalkyl, C₁-C₆hydroxyalkyl, or C₁-C₆aminoalkyl, wherein each of the alkyl, haloalkyl, hydroxyalkyl, or aminoalkyl is optionally substituted with 1 to 4 substituents independently selected from R^{e}; or
two R¹ are taken together to form an oxo; or
two R¹ are taken together to form a bond, C₁-C₃alkylene, or C₁-C₃heteroalkylene, wherein the alkylene or heteroalkylene is optionally substituted with 1 to 4 substituents independently selected from R^{e}; or
two R¹ are taken together to form a C₃-C₆cycloalkyl or 4 to 6-membered heterocycloalkyl, each of which is optionally substituted with 1 to 4 substituents independently selected from R^{e};
R⁹ is hydrogen, C₁-C₆alkyl, C₁-C₆haloalkyl, C₃-C₆cycloalkyl, or 4 to 6-membered heterocycloalkyl, wherein each alkyl, haloalkyl, cycloalkyl and heterocycloalkyl is independently substituted with 1 to 4 substituents selected from R^{e};
each Rₐ is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e}; and
each R^{e} is independently halogen, oxo, —CN, —OH, —S(═O)CH₃, —S(═O)₂CH₃, —CH₂S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁₋₆heteroalkyl, C₃-C₆cycloalkyl, or 4 to 6-membered heterocycloalkyl.

In some embodiments of Formula (I″), (I′), or (I),

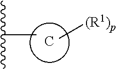

is

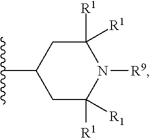

wherein,
each R¹ is independently hydrogen, halogen, —CN, —NO₂, —OH, —OR^{a}, —SH, —SR^{a}, —NR^{c}R^{d}, C₁-C₆alkyl, C₁-C₆haloalkyl, C₁-C₆hydroxyalkyl, or C₁-C₆aminoalkyl, wherein each of the alkyl, haloalkyl, hydroxyalkyl, or aminoalkyl is optionally substituted with 1 to 4 substituents independently selected from R^{e}; or
two R¹ are taken together to form a bond, C₁-C₃alkylene, or C₁-C₃heteroalkylene, wherein the alkylene or heteroalkylene is optionally substituted with 1 to 4 substituents independently selected from R^{e}; or
two R¹ are taken together to form a C₃-C₆cycloalkyl or 4 to 6-membered heterocycloalkyl, each of which is optionally substituted with 1 to 4 substituents independently selected from R^{e};
R⁹ is hydrogen, C₁-C₆alkyl, C₁-C₆haloalkyl, C₃-C₆cycloalkyl, or 4 to 6-membered heterocycloalkyl, wherein each alkyl, haloalkyl, cycloalkyl and heterocycloalkyl is independently substituted with 1 to 4 substituents selected from R^{e};
each Rₐ is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e}; and
each R^{e} is independently halogen, oxo, —CN, —OH, —S(═O)CH₃, —S(═O)₂CH₃, —CH₂S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6-membered heterocycloalkyl.

In some embodiments of Formula (I″), (I′), or (I),

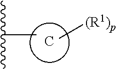

is

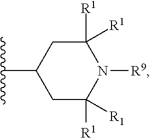

wherein,
each R^{b} is independently hydrogen, halogen, C₁-C₆alkyl, or C₁-C₆haloalkyl, wherein each of the alkyl, haloalkyl, hydroxyalkyl, or aminoalkyl is optionally substituted with 1 to 4 substituents independently selected from R^{e}; or
two R¹ are taken together to form a C₁-C₃alkylene or C₁-C₃heteroalkylene, wherein the alkylene or heteroalkylene is optionally substituted with 1 to 4 substituents independently selected from R^{e}; or
two R¹ are taken together to form a C₃-C₆cycloalkyl; and
R⁹ is hydrogen.

In some embodiments of Formula (I″), (I′), or (I),

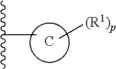

is

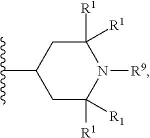

wherein,
each R^{b} is independently hydrogen or C₁-C₆alkyl; or
two R¹ are taken together to form a C₁-C₃alkylene; or
two R¹ are taken together to form a C₃-C₆cycloalkyl; and
R⁹ is hydrogen.

In some embodiments of Formula (I″), (I′), or (I),

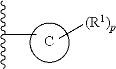

is

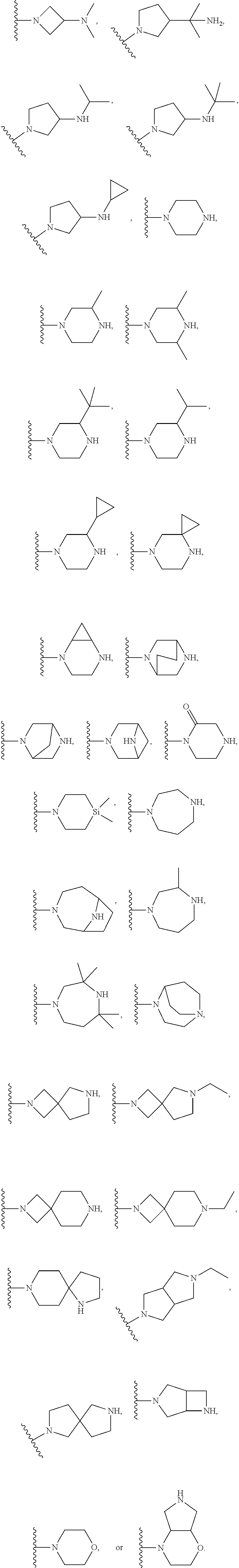

In some embodiments of Formula (I″), (I′), or (I),

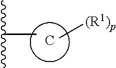

is

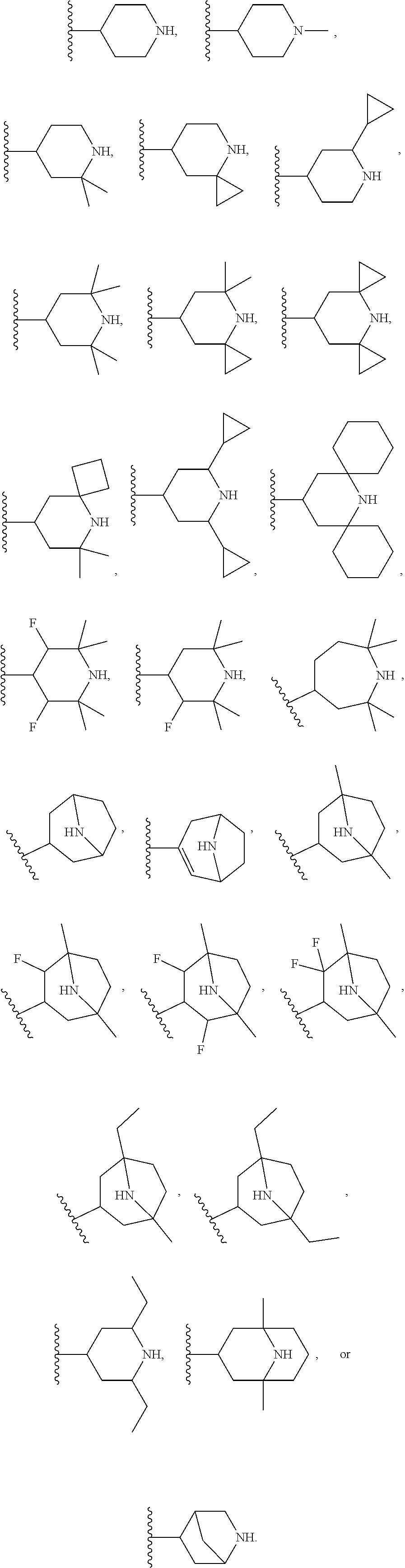

In some embodiments of Formula (I″), (I′), or (I),

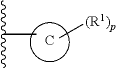

is or

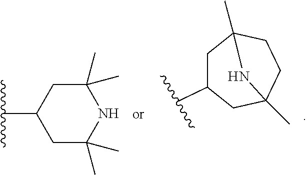 [SMILES: *C1CC(C)(C)NC(C)(C)C1 ; *C1CC2(C)CCC(C)(C1)N2]

In some embodiments of Formula (I″), (I′), or (I),

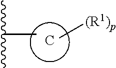

is

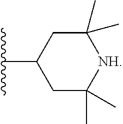 [SMILES: *C1CC(C)(C)NC(C)(C)C1]

In some embodiments of Formula (I″), (I′), or (I),

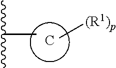

is

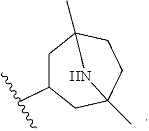 [SMILES: *C1CC2(C)CCC(C)(C1)N2]

In some embodiments, the compound is represented by Formula (II′), or a pharmaceutically acceptable salt or solvate thereof:

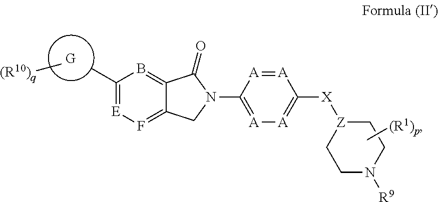

wherein,
ring G is a 5 to 10-membered heteroaryl;
each R¹ is independently hydrogen, halogen, —CN, —NO₂, —OH, —OR^{a}, —SH, —SR^{a}, —NR^{c}R^{d}, C₁-C₆alkyl, C₁-C₆haloalkyl, C₁-C₆hydroxyalkyl, or C₁-C₆aminoalkyl, wherein each of the alkyl, haloalkyl, hydroxyalkyl, or aminoalkyl is optionally substituted with 1 to 4 substituents independently selected from R^{e}; or
two R¹ are taken together to form an oxo; or
two R¹ are taken together to form a bond, C₁-C₃alkylene, or C₁-C₃heteroalkylene, wherein the alkylene or heteroalkylene is optionally substituted with 1 to 4 substituents independently selected from R^{e}; or
two R¹ are taken together to form a C₃-C₆cycloalkyl or 4 to 6-membered heterocycloalkyl, each of which is optionally substituted with 1 to 4 substituents independently selected from R;
X is bond, —CH₂—, —O—, —S—, or —NR³—, wherein
R³ is hydrogen, C₁-C₃alkyl, C₁-C₃hydroxyalkyl, C₁-C₃haloalkyl, or C₃-C₆ cycloalkyl;
Z is CH or N, when X is —O—, —S—, or —NR³—, then Z is CH;
each A is independently N or CR⁴, wherein at least two of A are N;
each R⁴ is independently hydrogen, halogen, —CN, —NO₂, —OH, —OR^{a}, —SH, —SR^{a}, —NR^{c}R^{d}, C₁-C₆alkyl, C₁-C₆haloalkyl, C₁-C₆hydroxyalkyl, or C₁-C₆aminoalkyl;
B is N or CR⁵;
E is N or CR⁷;
F is N or CR⁸;
R⁵, R⁷, and R⁸ are each independently hydrogen, halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, or C₁-C₆aminoalkyl; wherein each of the alkyl, alkene, alkyne, haloalkyl, and hydroxyalkyl, and aminoalkyl is optionally substituted with 1 to 4 substituents independently selected from R^{e};
R⁹ is hydrogen, C₁-C₆alkyl, C₁-C₆haloalkyl, C₃-C₆cycloalkyl, or 4 to 6-membered heterocycloalkyl, wherein each alkyl, haloalkyl, cycloalkyl and heterocycloalkyl is independently substituted with 1 to 4 substituents selected from R^{e};
each R¹⁰ is independently halogen, —CN, —NO₂, —OH, —OR^{a}, —NR^{c}R^{d}, C₁-C₆alkyl, C₁-C₆haloalkyl, C₁-C₆hydroxyalkyl, C₁-C₆heteroalkyl, C₁-C₆aminoalkyl, C₃-C₆cycloalkyl, or 4 to 6-membered heterocycloalkyl, wherein each of the alkyl, haloalkyl, hydroxyalkyl, heteroalkyl, cycloalkyl and heterocycloalkyl is optionally substituted with 1 to 4-substituents independently selected from R^{e}; or
two R¹⁰ are taken together to form an oxo;
each Rₐ is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{e} is independently halogen, oxo, —CN, —OH, —S(═O)CH₃, —S(═O)₂CH₃, —CH₂S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(O)NH₂, —C(O)NHCH₃, —C(O)N(CH₃)₂, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6-membered heterocycloalkyl;
p is 8; and
q is 0, 1, 2, or 3.

In some embodiments of Formula (I″), (I′), or (II′), two of A are N. In some embodiments of Formula (I″), (I′), or (II′), three of A are N. In some embodiments of Formula (I″), (I′), or (II′), each A is N. In some embodiments of Formula (I″), (I′), (I), (II), or (II′),

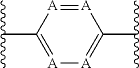

is

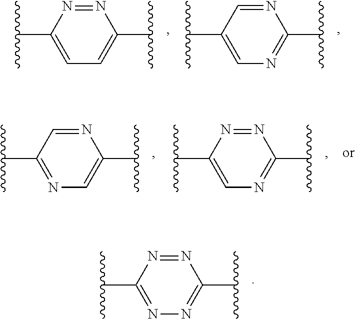

In some embodiments of Formula (I″), (I′), (I), (II), or (II′),

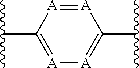

is

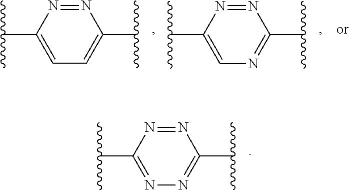

In some embodiments of Formula (I″), (I′), (I), (II), or (II′),

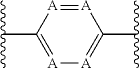

is

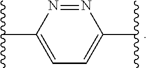 [SMILES: *c1ccc(*)nn1]

In some embodiments of Formula (I″), (I′), (I), (II), or (II′),

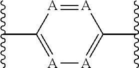

is

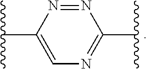 [SMILES: *c1cnc(*)nn1]

In some embodiments of Formula (I″), (I′), (I), (II), or (II′),

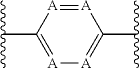

is

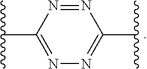 [SMILES: *c1nnc(*)nn1]

In some embodiments, the compound is represented by Formula (III′), or a pharmaceutically acceptable salt or solvate thereof:

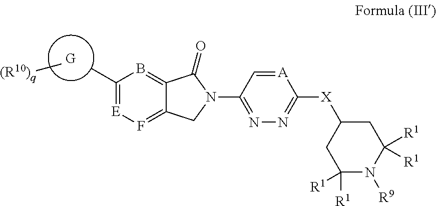

wherein,
ring G is a 5 to 10-membered heteroaryl;
each R¹ is independently hydrogen, halogen, —CN, —NO₂, —OH, —OR^{a}, —SH, —SR^{a}, —NR^{c}R^{d}, C₁-C₆alkyl, C₁-C₆haloalkyl, C₁-C₆hydroxyalkyl, or C₁-C₆aminoalkyl, wherein each of the alkyl, haloalkyl, hydroxyalkyl, or aminoalkyl is optionally substituted with 1 to 4 substituents independently selected from R^{e}; or
two R¹ are taken together to form a bond, C₁-C₃alkylene, or C₁-C₃heteroalkylene, wherein the alkylene or heteroalkylene is optionally substituted with 1 to 4 substituents independently selected from R^{e}; or
two R¹ are taken together to form a C₃-C₆cycloalkyl or 4 to 6-membered heterocycloalkyl, each of which is optionally substituted with 1 to 4 substituents independently selected from R;
X is —CH₂—, —O—, —S—, or —NR³—; wherein
R³ is hydrogen, C₁-C₃alkyl, C₁-C₃haloalkyl, C₁-C₃hydroxyalkyl, or C₃-C₆ cycloalkyl;
A is N or CH;
B is N or CR⁵;
E is N or CR⁷;
F is N or CR⁸;
R⁵, R⁷, and R⁸ are each independently hydrogen, halogen, —CN, —NO₂, —OH, —OR, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, or C₁-C₆aminoalkyl; wherein each of the alkyl, alkene, alkyne, haloalkyl, and hydroxyalkyl, and aminoalkyl is optionally substituted with 1 to 4 substituents independently selected from R^{e};
R⁹ is hydrogen, C₁-C₆alkyl, C₁-C₆haloalkyl, C₃-C₆cycloalkyl, or 4 to 6-membered heterocycloalkyl, wherein each alkyl, haloalkyl, cycloalkyl and heterocycloalkyl is independently substituted with 1 to 4 substituents selected from R^{e};
each R¹⁰ is independently halogen, —CN, —NO₂, —OH, —OR^{a}, —NR^{c}R^{d}, C₁-C₆alkyl, C₁-C₆haloalkyl, C₁-C₆hydroxyalkyl, C₁-C₆heteroalkyl, C₁-C₆aminoalkyl, C₃-C₆cycloalkyl, or 4 to 6-membered heterocycloalkyl, wherein each of the alkyl, haloalkyl, hydroxyalkyl, heteroalkyl, cycloalkyl and heterocycloalkyl is optionally substituted with 1 to 4-substituents independently selected from R; or
two R¹⁰ are taken together to form an oxo;
each Rₐ is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{e} is independently halogen, oxo, —CN, —OH, —S(═O)CH₃, —S(═O)₂CH₃, —CH₂S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(O)NH₂, —C(O)NHCH₃, —C(O)N(CH₃)₂, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6-membered heterocycloalkyl; and
q is 0, 1, 2, or 3.

In some embodiments of Formula (II), (II′), (III), or (III′), R⁹ is hydrogen, C₁-C₆alkyl, C₁-C₆haloalkyl, C₃-C₆cycloalkyl, or 4 to 6-membered heterocycloalkyl, wherein each alkyl, haloalkyl, cycloalkyl and heterocycloalkyl is independently substituted with 1 to 4 substituents selected from R^{e}. In some embodiments of Formula (II), (II′), (III), or (III′), R⁹ is hydrogen, C₁-C₆alkyl, or C₁-C₆haloalkyl. In some embodiments of Formula (II), (II′), (III), or (III′), R⁹ is hydrogen or C₁-C₆alkyl. In some embodiments of Formula (II), (II′), (III), or (III′), R⁹ is C₁-C₆haloalkyl. In some embodiments of Formula (II), (II′), (III), or (III′), R¹ is C₁-C₆alkyl. In some embodiments of Formula (II), (II′), (III), or (III′), R⁹ is hydrogen.

In some embodiments of Formula (II), (II), (III), or (III′), two R¹ are taken together to form a bond, C₁-C₃alkylene or C₁-C₃heteroalkylene, wherein the alkylene or heteroalkylene is optionally substituted with 1 to 4 substituents independently selected from R^{e}. In some embodiments of Formula (II), (II), (III), or (III′), two R¹ are taken together to form a bond. In some embodiments of Formula (II), (II′), (III), or (III′), two R¹ are taken together to form a C₁-C₃alkylene. In some embodiments of Formula (II), (II′), (III), or (III′), two R¹ are taken together to form or a C₁-C₃heteroalkylene.

In some embodiments, the compound of Formula (III′) has the structure of Formula (IIIa′), or a pharmaceutically acceptable salt or solvate thereof:

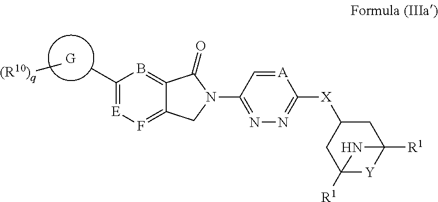

wherein,
Y is C₁-C₃alkylene
ring G is a 5 to 10-membered heteroaryl;
each R¹ is independently hydrogen, halogen, —CN, —NO₂, —OH, —OR^{a}, —SH, —SR^{a}, —NR^{c}R^{d}, C₁-C₆alkyl, C₁-C₆haloalkyl, C₁-C₆hydroxyalkyl, or C₁-C₆aminoalkyl, wherein each of the alkyl, haloalkyl, hydroxyalkyl, or aminoalkyl is optionally substituted with 1 to 4 substituents independently selected from R^{e}; or
X is —CH₂—, —O—, or —NR³—, wherein
R³ is hydrogen, C₁-C₃alkyl, C₁-C₃hydroxyalkyl, C₁-C₃haloalkyl, or C₃-C₆ cycloalkyl;
A is N or CH;
B is N or CR⁵;
E is N or CR⁷;
F is N or CR¹;
R⁵, R⁷, and R⁸ are each independently hydrogen, halogen, —CN, —NO₂, —OH, —OR, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, or C₁-C₆aminoalkyl; wherein each of the alkyl, alkene, alkyne, haloalkyl, and hydroxyalkyl, and aminoalkyl is optionally substituted with 1 to 4 substituents independently selected from R^{e};
each R¹⁰ is independently halogen, —CN, —NO₂, —OH, —OR, —NR^{c}R^{d}, C₁-C₆alkyl, C₁-C₆haloalkyl, C₁-C₆hydroxyalkyl, C₁-C₆heteroalkyl, C₁-C₆aminoalkyl, C₃-C₆cycloalkyl, or 4 to 6-membered heterocycloalkyl, wherein each of the alkyl, haloalkyl, hydroxyalkyl, heteroalkyl, cycloalkyl and heterocycloalkyl is optionally substituted with 1 to 4-substituents independently selected from R^{e}; or
two R¹⁰ are taken together to form an oxo;
each Rₐ is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R*;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{e} is independently halogen, oxo, —CN, —OH, —S(═O)CH₃, —S(═O)₂CH₃, —CH₂S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(O)NH₂, —C(O)NHCH₃, —C(O)N(CH₃)₂, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6-membered heterocycloalkyl; and
q is 0, 1, 2, or 3.

In some embodiments of Formula (IIIa′), Y is —CH₂CH₂CH₂—. In some embodiments of Formula (IIIa′), Y is —CH₂CH₂—. In some embodiments of Formula (IIIa′), Y is —CH₂—.

In some embodiments, the compound of Formula (III′) has the structure of Formula (IIIb′), or a pharmaceutically acceptable salt or solvate thereof:

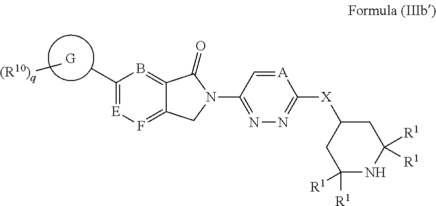

wherein,
ring G is a 5 to 10-membered heteroaryl;
each R¹ is independently hydrogen, halogen, —CN, —NO₂, —OH, —OR^{a}, —SH, —SR^{a}, —NR^{c}R^{d}, C₁-C₆alkyl, C₁-C₆haloalkyl, C₁-C₆hydroxyalkyl, or C₁-C₆aminoalkyl, wherein each of the alkyl, haloalkyl, hydroxyalkyl, or aminoalkyl is optionally substituted with 1 to 4 substituents independently selected from R^{e}; or
two R¹ are taken together to form a C₃-C₆cycloalkyl or 4 to 6-membered heterocycloalkyl, each of which is optionally substituted with 1 to 4 substituents independently selected from R^{e};
X is —CH₂—, —O—, —S—, or —NR³—, wherein R³ is hydrogen, C₁-C₃alkyl, C₁-C₃hydroxyalkyl, C₁-C₃haloalkyl, or C₃-C₆ cycloalkyl;
A is N or CH;
B is N or CR⁵;
E is N or CR⁷;
F is N or CR⁸;
R⁵, R⁷, and R⁸ are each independently hydrogen, halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, or C₁-C₆aminoalkyl; wherein each of the alkyl, alkene, alkyne, haloalkyl, and hydroxyalkyl, and aminoalkyl is optionally substituted with 1 to 4 substituents independently selected from R^{e};
each R¹⁰ is independently halogen, —CN, —NO₂, —OH, —OR^{a}, —NR^{c}R^{d}, C₁-C₆alkyl, C₁-C₆haloalkyl, C₁-C₆hydroxyalkyl, C₁-C₆heteroalkyl, C₁-C₆aminoalkyl, C₃-C₆cycloalkyl, or 4 to 6-membered heterocycloalkyl, wherein each of the alkyl, haloalkyl, hydroxyalkyl, heteroalkyl, cycloalkyl and heterocycloalkyl is optionally substituted with 1 to 4-substituents independently selected from R^{e}; or
two R¹⁰ are taken together to form an oxo;
each Rₐ is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R;
each R^{e} is independently halogen, oxo, —CN, —OH, —S(═O)CH₃, —S(═O)₂CH₃, —CH₂S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(O)NH₂, —C(O)NHCH₃, —C(O)N(CH₃)₂, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6-membered heterocycloalkyl; and
q is 0, 1, 2, or 3.

In some embodiments of Formula (II′), when X is —O—, —S—, or —NR³—, then Z is CH. In some embodiments of Formula (II) or (II′), when X is bond, then Z is CH or N. In some embodiments of Formula (II) or (II′), when X is bond, then Z is N. In some embodiments of Formula (II) or (II′), when X is —CH₂—, then Z is CH or N. In some embodiments of Formula (II) or (II′), when X is —CH₂—, then Z is CH. In some embodiments of Formula (II) or (II′), when X is —CH₂—, then Z is N.

In some embodiments of Formula (I″), (I′), or (II′), X is —O—, —S—, or —NR³—. In some embodiments of Formula (I″), (I′), (I), (II), (II′), (III), (III′), (IIIa), (IIIa′), (IIIb), or (IIIb′), X is —O— or —NR³—. In some embodiments of Formula (I″), (I′), or (II′), X is —S—. In some embodiments of Formula (I″), (I′), (I), (II), (II′) (III), (III′) (IIIa), (IIIa′), (IIIb), or (IIIb′), X is —O—. In some embodiments of Formula (I″), (I′), (I), (II), (II′), (III), (II′), (IIIa), (IIIa′), (IIIb), or (IIIb′), X is —NR³—. In some embodiments of Formula (I″), (I′), (I), (II), (II′), (III), (II′), (IIIa), (IIIa′), (IIIb), or (IIIb′), X is —NH—. In some embodiments of Formula (I″), (I′), (I), (II), (II′), (III), (II′), (IIIa), (IIIa′), (IIIb), or (IIIb′), X is —NCH₃—. In some embodiments of Formula (I′), (I), (II), (III), (IIIa), or (IIIb), X is —CH₂. In some embodiments of Formula (I″), X is —OCH₂—.

In some embodiments of Formula (I″), (I′), (I), (II), or (II′), X is absent.

In some embodiments of Formula (I″), (I′), (I), (II), (II′), (III), (III′), (IIIa), (IIIa′), (IIIb), or (IIIb′), R³ is hydrogen, C₁-C₃alkyl, or C₁-C₃hydroxyalkyl. In some embodiments of Formula (I″), (I′), (II′), (IIIa′), or (IIIb), R³ is C₃-C₆cycloaalkyl. In some embodiments of Formula (I″), (I′), (II′), (III′), (IIIa′), or (IIIb′), R³ is cyclopropyl or cyclobutyl. In some embodiments of Formula (I″), (I′), (II′), (III′), (IIIa′), or (IIIb′), R³ is cyclopropyl. In some embodiments of Formula (I″), (I′), (I), (II), (II′), (III), (III′), (IIIa), (IIIa′), (IIIb), or (IIIb′), R³ is hydrogen or C₁-C₃alkyl. In some embodiments of Formula (I″), (I′), (I), (II), (II′), (III), (III′), (IIIa), (IIIa′), (IIIb), or (IIIb′), R³ is C₁-C₃hydroxyalkyl. In some embodiments of Formula (I″), (I′), (I), (II), (II′), (III), (III′), (IIIa), (IIIa′), (IIIb), or (IIIb′), R³ is C₁-C₃haloalkyl. In some embodiments of Formula (I″), (I′), (I), (II), (II′), (III), (III′), (IIIa), (IIIa′), (IIIb), or (IIIb′), R³ is C₁-C₃alkyl. In some embodiments of Formula (I″), (I′), (I), (II), (II′), (III), (III′), (IIIa), (IIIa′), (IIIb), or (IIIb′), R³ is hydrogen or methyl. In some embodiments of Formula (I″), (I′), (I), (II), (II′), (III), (III′), (IIIa), (IIIa′), (IIIb), or (IIIb′), R³ is methyl. In some embodiments of Formula (I″), (I′), (I), (II), (II′), (III), (III′), (IIIa), (IIIa′), (IIIb), or (IIIb′), R³ is hydrogen.

In some embodiments, the compound is represented by Formula (IV′), or a pharmaceutically acceptable salt or solvate thereof:

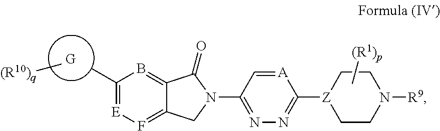

wherein,
ring G is a 5 to 10-membered heteroaryl;
each R¹ is independently hydrogen, halogen, —CN, —NO₂, —OH, —OR^{a}, —SH, —SR^{a}, —NR^{c}R^{d}, C₁-C₆alkyl, C₁-C₆haloalkyl, C₁-C₆hydroxyalkyl, or C₁-C₆aminoalkyl, wherein each of the alkyl, haloalkyl, hydroxyalkyl, or aminoalkyl is optionally substituted with 1 to 4 substituents independently selected from R^{e}; or
two R¹ are taken together to form an oxo; or
two R¹ are taken together to form a bond, C₁-C₃alkylene, or C₁-C₃heteroalkylene, wherein the alkylene or heteroalkylene is optionally substituted with 1 to 4 substituents independently selected from R^{e}; or
two R¹ are taken together to form a C₃-C₆cycloalkyl or 4 to 6-membered heterocycloalkyl, each of which is optionally substituted with 1 to 4 substituents independently selected from R;
Z is CH or N;
A is N or CH;
B is N or CR⁵;
E is N or CR⁷;
F is N or CR⁸;
R⁵, R⁷, and R⁸ are each independently hydrogen, halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, or C₁-C₆aminoalkyl; wherein each of the alkyl, alkene, alkyne, haloalkyl, and hydroxyalkyl, and aminoalkyl is optionally substituted with 1 to 4 substituents independently selected from R^{e};
R⁹ is hydrogen, C₁-C₆alkyl, C₁-C₆haloalkyl, C₃-C₆cycloalkyl, or 4 to 6-membered heterocycloalkyl, wherein each alkyl, haloalkyl, cycloalkyl and heterocycloalkyl is independently substituted with 1 to 4 substituents selected from R^{e};
each R¹⁰ is independently halogen, —CN, —NO₂, —OH, —OR^{a}, —NR^{c}R^{d}, C₁-C₆alkyl, C₁-C₆haloalkyl, C₁-C₆hydroxyalkyl, C₁-C₆heteroalkyl, C₁-C₆aminoalkyl, C₃-C₆cycloalkyl, or 4 to 6-membered heterocycloalkyl, wherein each of the alkyl, haloalkyl, hydroxyalkyl, heteroalkyl, cycloalkyl and heterocycloalkyl is optionally substituted with 1 to 4-substituents independently selected from R^{e}; or
two R¹⁰ are taken together to form an oxo;
each Rₐ is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{e} is independently halogen, oxo, —CN, —OH, —S(═O)CH₃, —S(═O)₂CH₃, —CH₂S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(O)NH₂, —C(O)NHCH₃, —C(O)N(CH₃)₂, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6-membered heterocycloalkyl;
p is 8; and
q is 0, 1, 2, or 3.

In some embodiments of Formula (II), (II′), (IV), or (IV′), Z is N. In some embodiments of Formula (II), (II′), (IV), or (IV′), Z is CH.

In some embodiments of Formula (I″), (I′), (I), (II), or (II′), at least one of A is N.

In some embodiments of Formula (I″), (I′), (I), (II), (II′), (III), (III′), (IIIa), (IIIa′), (IIIb), (IIIb), (IV), or (IV′), each A is N. In some embodiments of Formula (I″), (I′), (I), (II), (II′), (III), (III′), (IIIa), (IIIa′), (IIIb), (IIIb′), (IV), or (IV′), each A is independently CR⁴.

In some embodiments of Formula (I″), (I′), (I), (II), (II′), (III), (III′), (IIIa), (IIIa′), (IIIb), (IIIb′), (IV), or (IV′), each A is independently N or CH. In some embodiments of Formula (I″), (I′), (I), (II), (II′), (III), (III′), (IIIa), (IIIa′), (IIIb), (IIIb′), (IV), or (IV′), each A is CH.

In some embodiments of Formula (I″), (I′), (I), (II), (II′), (III), (III′), (IIIa), (IIIa′), (IIIb), (IIIb′), (IV′), or (IV′), B is CR⁵. In some embodiments of Formula (I″), (I′), (I), (II), (II′), (III), (III′), (IIIa), (IIIa′), (IIIb), (IIIb′), (IV), or (IV′), B is CH. In some embodiments of Formula (I″), (I′), (I), (II), (II′), (III), (III′), (IIIa), (IIIa′), (IIIb), (IIIb′), (IV), or (IV′), B is N.

In some embodiments of Formula (I″), (I′), (I), (II), (II′), (III), (III′), (IIIa), (IIIa′), (IIIb), (IIIb′), (IV), or (IV′), E is CR⁷. In some embodiments of Formula (I″), (I′), (I), (II), (II′), (III), (III′), (IIIa), (IIIa′), (IIIb), (IIIb′), (IV), or (IV′), E is CH. In some embodiments of Formula (I″), (I′), (I), (II), (II′), (III), (III′), (IIIa), (IIIa′), (IIIb), (IIIb′), (IV), or (IV′), E is N.

In some embodiments of Formula (I″), (I′), (I), (II), (II′), (III), (III′), (IIIa), (IIIa′), (IIIb), (IIIb′), (IV), or (IV′), F is CR⁸. In some embodiments of Formula (I″), (I′), (I), (II), (II′), (III), (III′), (IIIa), (IIIa′), (IIIb), (IIIb′), (IV), or (IV′), F is CH. In some embodiments of Formula (I″), (I′), (I), (II), (II′), (III), (III′), (IIIa), (IIIa′), (IIIb), (IIIb′), (IV), or (IV′), F is N.

In some embodiments of Formula (I″), (I′), (I), (II), (II′), (III), (III′), (IIIa), (IIIa′), (IIIb), (IIIb′), (IV), or (IV′), each R¹ is independently hydrogen, halogen, —CN, —NO₂, —OH, —OR^{a}, —SH, —SR^{a}, —NR^{c}R^{d}, C₁-C₆alkyl, C₁-C₆haloalkyl, C₁-C₆hydroxyalkyl, or C₁-C₆aminoalkyl, wherein each of the alkyl, haloalkyl, hydroxyalkyl, or aminoalkyl is optionally substituted with 1 to 4 substituents independently selected from R^{e}. In some embodiments of Formula (I″), (I′), (I), (II), (II′), (III), (III′), (IIIa), (IIIa′), (IIIb), (IIIb′), (IV), or (IV′), each R^{b} is independently hydrogen, halogen, C₁-C₆alkyl, or C₁-C₆haloalkyl, wherein each of the alkyl, haloalkyl, hydroxyalkyl, or aminoalkyl is optionally substituted with 1 to 4 substituents independently selected from R^{e}. In some embodiments of Formula (I″), (I′), (I), (II), (II′), (III), (III′), (IIIa), (IIIa′), (IIIb), (IIIb′), (IV), or (IV′), each R^{b} is independently hydrogen, halogen, or C₁-C₆alkyl. In some embodiments of Formula (I″), (I′), (I), (II), (II′), (III), (III′), (IIIa), (IIIa′), (IIIb), (IIIb′), (IV), or (IV′), each R¹ is independently C₁-C₆alkyl, or C₁-C₆haloalkyl. In some embodiments of Formula (II), (II′), (III), (III′), (IIIa), (IIIa′), (IIIb), (IIIb′), (IV), or (IV′), each R¹ is independently —CH₃, —CH₂CH₃, —CH(CH₃)₂, —CF₃, or —CHF₂. In some embodiments of Formula (I″), (I′), (I), (II), (II′), (III), (III′), (IIIa), (IIIa′), (IIIb), (IIIb′), (IV), or (IV′), each R^{b} is independently hydrogen, —CH₃, —CH₂CH₃, or —CH(CH₃)₂. In some embodiments of Formula (I″), (I′), (I), (II), (II′), (III), (III′), (IIIa), (IIIa′), (IIIb), (IIIb′), (IV), or (IV′), each R¹ is independently halogen. In some embodiments of Formula (I″), (I′), (I), (II), (II′), (III), (III′), (IIIa), (IIIa′), (IIIb), (IIIb′), (IV), or (IV′), each R¹ is independently C₁, Br, or F. In some embodiments of Formula (I″), (I′), (I), (II), (II′), (III), (III′), (IIIa), (IIIa′), (IIIb), (IIIb′), (IV), or (IV′), each R¹ is independently F. In some embodiments of Formula (I″), (I′), (I), (II), (II′), (III), (III′), (IIIa), (IIIa′), (IIIb), (IIIb′), (IV), or (IV′), each R^{b} is independently hydrogen.

In some embodiments of Formula (I″), (I′), (I), (II), (II′), (III), (III′), (IIIb), (IIIb′), (IV), or (IV′), two R¹ are taken together to form a C₃-C₆cycloalkyl. In some embodiments of Formula (I″), (I′), (I), (II), (II′), (III), (III′), (IIIb), (IIIb′), (IV), or (IV′), two R¹ are taken together to form a cyclopropyl, cyclobutyl, cyclopentyl, or cyclohexyl. In some embodiments of Formula (I″), (I′), (I), (II), (II′), (III), (III′), (IIIb), (IIIb′), (IV), or (IV′), two R¹ are taken together to form a cyclopropyl. In some embodiments of Formula (I″), (I′), (I), (II), (II′), (III), (III′) (IIIb), (IIIb′), (IV), or (IV′), two R¹ are taken together to form a cyclobutyl. In some embodiments of Formula (I″), (I′), (I), (II), (II′), (III), (III′), (IIIb), (IIIb′), (IV), or (IV′), two R¹ are taken together to form a cyclopentyl. In some embodiments of Formula (I″), (I′), (I), (II), (II′), (III), (III′), (IIIb), (IIIb′), (IV), or (IV′), two R¹ are taken together to form a cyclohexyl.

In some embodiments of Formula (I″), (I′), (I), (II), (II′), (III), (III′), (IIIb), (IIIb′), (IV), or (IV′), two R¹ are taken together to form an oxo.

In some embodiments of Formula (I″), (I′), (I), (II), or (II′), each R⁴ is independently hydrogen, halogen, —CN, —NO₂, —OH, —OR^{a}, —SH, —SR, —NR^{c}R^{d}, C₁-C₆alkyl, C₁-C₆haloalkyl, C₁-C₆hydroxyalkyl, or C₁-C₆aminoalkyl. In some embodiments of Formula (I″), (I′), (I), (II), or (II′), each R⁴ is independently hydrogen, halogen, —CN, —OH, —OR^{a}, —NR^{c}R^{d}, C₁-C₆alkyl, C₁-C₆haloalkyl, C₁-C₆hydroxyalkyl, or C₁-C₆aminoalkyl. In some embodiments of Formula (I″), (I′), (I), (II), or (II′), each R⁴ is independently hydrogen, halogen, —OH, —OR^{a}, C₁-C₆alkyl, C₁-C₆haloalkyl, or C₁-C₆hydroxyalkyl. In some embodiments of Formula (I″), (I′), (I), (II), or (II′), each R⁴ is independently hydrogen, halogen, or C₁-C₆alkyl. In some embodiments of Formula (I″), (I′), (I), (II), or (II′), each R⁴ is independently halogen. In some embodiments of Formula (I″), (I′), (I), (II), or (II′), each R⁴ is independently C₁-C₆alkyl. In some embodiments of Formula (I″), (I′), (I), (II), or (II′), each R⁴ is independently hydrogen.

In some embodiments of Formula (I″), R³ and one of R⁴ are taken together to form a 5 or 6-membered heterocycloalkylene or 5 or 6-membered heteroarylene.

In some embodiments of Formula (I″), R⁵, R⁶, R⁷ or R⁸ are independently hydrogen, halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, C₁-C₆aminoalkyl, C₃-C₆cycloalkyl, 4 to 6-membered heterocycloalkyl, or 5 to 10-membered heteroaryl; wherein each of the alkyl, alkene, alkyne, haloalkyl, hydroxyalkyl, aminoalkyl, cycloalkyl, heterocycloalkyl, or heteroaryl is optionally substituted with 1 to 4 substituents independently selected from R; and wherein at least one of R⁵, R⁶, R⁷ or R⁸ is C₂-C₆alkene, C₃-C₆cycloalkyl, 4 to 6-membered heterocycloalkyl, or 5 to 8 membered heteroaryl, wherein the alkene, cycloalkyl, heterocycloalkyl and heteroaryl is optionally substituted with 1 to 4 substituents selected from R^{e}. In some embodiments of Formula (I″), R⁵, R⁶, R⁷ or R⁸ are independently hydrogen, halogen, —CN, —NO₂, C₁-C₆alkyl, C₂-C₆alkene, C₂-C₆alkyne, C₁-C₆haloalkyl, C₃-C₆cycloalkyl, 4 to 6-membered heterocycloalkyl, or 5 to 10-membered heteroaryl. In some embodiments of Formula (I″), R⁵, R⁶, R⁷ or R⁸ are independently hydrogen, halogen, —CN, C₃-C₆cycloalkyl, or 5 to 10-membered heteroaryl.

In some embodiments of Formula (I″), R⁵ is 5 to 10-membered heteroaryl; and R⁶, R⁷ and R¹ are independently hydrogen, halogen, —CN, —NO₂, C₁-C₆alkyl, C₂-C₆alkene, C₂-C₆alkyne, or C₁-C₆haloalkyl.

In some embodiments of Formula (I″), R⁶ is 5 to 10-membered heteroaryl; and R⁵, R⁷ and R¹ are independently hydrogen, halogen, —CN, —NO₂, C₁-C₆alkyl, C₂-C₆alkene, C₂-C₆alkyne, or C₁-C₆haloalkyl.

In some embodiments of Formula (I″), R⁷ is 5 to 10-membered heteroaryl; and R⁵, R⁶ and R¹ are independently hydrogen, halogen, —CN, —NO₂, C₁-C₆alkyl, C₂-C₆alkene, C₂-C₆alkyne, or C₁-C₆haloalkyl.

In some embodiments of Formula (I″), R¹ is 5 to 10-membered heteroaryl; and R⁵, R⁶ and R⁷ are independently hydrogen, halogen, —CN, —NO₂, C₁-C₆alkyl, C₂-C₆alkene, C₂-C₆alkyne, or C₁-C₆haloalkyl.

In some embodiments of Formula (I″), B is CR⁵, wherein R¹ is C₂-C₆alkene, C₃-C₆cycloalkyl, 4 to 6-membered heterocycloalkyl, or 5 to 10-membered heteroaryl, wherein the alkene, cycloalkyl, heterocycloalkyl and heteroaryl is optionally substituted with 1 to 4 substituents selected from R^{e}; and F is N.

In some embodiments of Formula (I″), (I′), (II′), (III′), (IIIa′), (IIIb′), or (IV′), D is CR⁶, wherein R⁶ is C₂-C₆alkene, C₃-C₆cycloalkyl, 4 to 6-membered heterocycloalkyl, or 5 to 10-membered heteroaryl, wherein the alkene, cycloalkyl, heterocycloalkyl and heteroaryl is optionally substituted with 1 to 4 substituents selected from R^{e}; and E is N. In some embodiments of Formula (I″), (I′), (II′), (III′), (IIIa′), (IIIb′), or (IV′), D is CR⁶, wherein R⁶ is C₂-C₆alkene, C₃-C₆cycloalkyl, 4 to 6-membered heterocycloalkyl, or 5 to 10-membered heteroaryl, wherein the alkene, cycloalkyl, heterocycloalkyl and heteroaryl is optionally substituted with 1 to 4 substituents selected from R^{e}; and F is N. In some embodiments of Formula (I″), (I′), (II′), (III′), (IIIa′), (IIIb′), or (IV′), D is CR⁶, wherein R⁶ is C₂-C₆alkene, C₃-C₆cycloalkyl, 4 to 6-membered heterocycloalkyl, or 5 to 10-membered heteroaryl, wherein the alkene, cycloalkyl, heterocycloalkyl and heteroaryl is optionally substituted with 1 to 4 substituents selected from R^{e}; B is N; and F is N. In some embodiments of Formula (I″), (I′), (II′) (III′), (IIIa′), (IIIb′), or (IV′), D is CR⁶, wherein R⁶ is C₂-C₆alkene, C₃-C₆cycloalkyl, 4 to 6-membered heterocycloalkyl, or 5 to 10-membered heteroaryl, wherein the alkene, cycloalkyl, heterocycloalkyl and heteroaryl is optionally substituted with 1 to 4 substituents selected from R^{e}; E is N; and F is N.

In some embodiments of Formula (I″), F is CR⁸, wherein R⁸ is C₂-C₆alkene, C₃-C₆cycloalkyl, 4 to 6-membered heterocycloalkyl, or 5 to 10-membered heteroaryl, wherein the alkene, cycloalkyl, heterocycloalkyl and heteroaryl is optionally substituted with 1 to 4 substituents selected from R^{e}; and E is N.

In some embodiments of Formula (I″), (I′), (I), (II), (II′), (III), (III′), (IIIa), (IIIa′), (IIIb), (IIIb′), (IV), or (IV′), R⁵ is hydrogen, halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, or C₁-C₆aminoalkyl; wherein each of the alkyl, haloalkyl, and hydroxyalkyl, and aminoalkyl is optionally substituted with 1 to 4 substituents is hydrogen, halogen, —CN, —NO₂, —OH, —OR, —SH, —SR, C₁-C₆alkyl, C₁-C₆haloalkyl, C₁-C₆hydroxyalkyl, or C₁-C₆aminoalkyl; wherein each of the alkyl, alkene, alkyne, haloalkyl, and hydroxyalkyl, and aminoalkyl is optionally substituted with 1 to 4 substituents independently selected from R^{e}. In some embodiments of Formula (I″), (I′), (I), (II), (II′), (III), (III′), (IIIa), (IIIa′), (IIIb), (IIIb′), (IV), or (IV′), R⁵ is hydrogen, halogen, C₁-C₆alkyl, C₁-C₆haloalkyl, C₁-C₆hydroxyalkyl, or C₁-C₆aminoalkyl. In some embodiments of Formula (I″), (I′), (I), (II), (II′), (III), (III′), (IIIa), (IIIa′), (IIIb), (III′), (IV), or (IV′), R⁵ C₁-C₆alkyl. In some embodiments of Formula (I″), (I′), (I), (II), (II′), (III), (III′), (IIIa), (IIIa′), (IIIb), (IIIb′), (IV), or (IV′), R⁵ is C₁-C₆haloalkyl. In some embodiments of Formula (I″), (I′), (I), (II), (II′), (III), (III′), (IIIa), (IIIa′), (IIIb), (IIIb′), (IV), or (IV′), R⁵ is C₁-C₆hydroxyalkyl. In some embodiments of Formula (I″), (I′), (I), (II), (II′), (III), (III′), (IIIa), (IIIa′), (IIIb), (IIIb′), (IV), or (IV′), R⁵ is halogen or hydrogen. In some embodiments of Formula (I″), (I′), (I), (II), (II′), (III), (III′), (IIIa), (IIIa′), (III), (III′), (IV), or (IV′), R⁵ is halogen. In some embodiments of Formula (I″), (I′), (I), (II), (II′), (III), (III′), (IIIa), (IIIa′), (IIIb), (IIIb′), (IV), or (IV′), R⁵ is hydrogen. In some embodiments of Formula (I″), (I′), (I), (II), (II′), (III), (III′), (IIIa), (IIIa′), (IIIb), (IIIb′), (IV), or (IV′), R⁵ is fluorine. In some embodiments of Formula (I″), (I′), (I), (II), (II′), (III), (III′), (IIIa), (IIIa′), (III), (III′), (IV), or (IV′), R⁵ is chlorine.

In some embodiments of Formula (I″), (I′), (I), (II), (II′), (III), (III′), (IIIa), (IIIa′), (IIIb), (IIIb′), (IV), or (IV′), R⁶ is hydrogen, halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, or C₁-C₆aminoalkyl; wherein each of the alkyl, alkene, alkyne, haloalkyl, and hydroxyalkyl, and aminoalkyl is optionally substituted with 1 to 4 substituents independently selected from R^{e}. In some embodiments of Formula (I″), (I′), (I), (II), (II′), (III), (III′), (IIIa), (IIIa′), (IIIb), (IIIb′), (IV), or (IV′), R⁶ is hydrogen, halogen, —CN, —NO₂, —OH, —OR^{a}, —SH, —SR^{a}, C₁-C₆alkyl, C₁-C₆haloalkyl, C₁-C₆hydroxyalkyl, or C₁-C₆aminoalkyl; wherein each of the alkyl, haloalkyl, and hydroxyalkyl, and aminoalkyl is optionally substituted with 1 to 4 substituents independently selected from R^{e}. In some embodiments of Formula (I″), (I′), (I), (II), (II′), (III), (III′), (IIIa), (IIIa′), (IIIb), (IIIb′), (IV), or (IV′), R⁶ is hydrogen, halogen, C₁-C₆alkyl, C₁-C₆haloalkyl, C₁-C₆hydroxyalkyl, or C₁-C₆aminoalkyl. In some embodiments of Formula (I″), (I′), (I), (II), (II′), (III), (III′), (IIIa), (IIIa′), (IIIb), (IIIb′), (IV), or (IV′), R⁶ is C₁-C₆alkyl. In some embodiments of Formula (I″), (I′), (I), (II), (II′), (III), (III′), (IIIa), (IIIa′), (III), (III′), (IV), or (IV′), R⁶ is C₁-C₆haloalkyl. In some embodiments of Formula (I″), (I′), (I), (II), (II′), (III), (III), (IIIa), (IIIa′), (IIIb), (IIIb′), (IV), or (IV′), R⁶ is C₁-C₆hydroxyalkyl. In some embodiments of Formula (I″), (I′), (I), (II), (II′), (III), (III′), (IIIa), (IIIa′), (IIIb), (IIIb′), (IV), or (IV′), R^{b} is halogen or hydrogen. In some embodiments of Formula (I″), (I′), (I), (II), (II′), (III), (III′), (IIIa), (IIIa′), (IIIb), (IIIb′), (IV), or (IV′), R⁶ is halogen. In some embodiments of Formula (I″), (I′), (I), (II), (II′), (III), (III′), (IIIa), (IIIa′), (IIIb), (IIIb′), (IV), or (IV′), R^{b} is hydrogen.

In some embodiments of Formula (I″) (I′), (I), (II), (II′), (III), (III′), (IIIa), (IIIa′), (IIIb), (IIIb′), (IV), or (IV′), R⁷ is hydrogen, halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, or C₁-C₆aminoalkyl; wherein each of the alkyl, alkene, alkyne, haloalkyl, and hydroxyalkyl, and aminoalkyl is optionally substituted with 1 to 4 substituents independently selected from R^{e}. In some embodiments of Formula (I″), (I′), (I), (II), (II′), (III), (III′), (IIIa), (IIIa′), (IIIb), (IIIb′), (IV), or (IV′), R⁷ is hydrogen, halogen, —CN, —NO₂, —OH, —OR^{a}, —SH, —SR^{a}, C₁-C₆alkyl, C₁-C₆haloalkyl, C₁-C₆hydroxyalkyl, or C₁-C₆aminoalkyl; wherein each of the alkyl, haloalkyl, and hydroxyalkyl, and aminoalkyl is optionally substituted with 1 to 4 substituents independently selected from R^{e}. In some embodiments of Formula (I″), (I′), (I), (II), (II′), (III), (III′), (IIIa), (IIIa′), (IIIb), (IIIb′), (IV), or (IV′), R⁷ is hydrogen, halogen, C₁-C₆alkyl, C₁-C₆haloalkyl, C₁-C₆hydroxyalkyl, or C₁-C₆aminoalkyl. In some embodiments of Formula (I″), (I′), (I), (II), (II′), (III), (III′), (IIIa), (IIIa′), (IIIb), (IIIb′), (IV), or (IV′), R⁷ is C₁-C₆alkyl. In some embodiments of Formula (I″), (I), (I), (II), (II′), (III), (III′), (IIIa), (IIIa′), (IIIb), (IIIb′), (IV), or (IV′), R⁷ is C₁-C₆haloalkyl. In some embodiments of Formula (I), (I′), (I, II), (II′), (IIIb), (III′), (IV), or (IV′), R⁷ is C₁-C₆hydroxyalkyl. In some embodiments of Formula (I″), (I′), (I), (II), (II′), (III), (III′), (IIIa), (IIIa′), (IIIb), (IIIb′), (IV), or (IV′), R⁷ is halogen or hydrogen. In some embodiments of Formula (I″), (I), ( ), (II), (II′), (III), (III′), (IIIa), (IIIa′), (IIIb), (IIIb′), (IV), or (IV′), R⁷ is halogen. In some embodiments of Formula (I″), (I′), (I), (II), (II′), (III), (III′), (IIIa), (IIIb), (IIIb′), (IV), or (IV′), R⁷ is hydrogen. In some embodiments of Formula (I″), (I′), (I), (II), (II′), (III), (III′), (IIIa), (IIIa′), (IIIb), (IIIb′), (IV), or (IV′), R⁷ is flourine. In some embodiments of Formula (I″), (I′), (I), (II), (II′), (III), (III′), (IIIa), (IIIa′), (IIIb), (IIIb′), (IV), or (IV′), R⁷ is chlorine. In some embodiments of Formula (I″), (I′), (I), (II), (II′), (III), (III′), (IIIa), (IIIa′), (IIIb), (IIIb′), (IV), or (IV′), R⁷ is hydroxyl. In some embodiments of Formula (I′), (I), (II), (III), (IIIa), (IIIb), or (IV), R⁷ is methoxy.

In some embodiments of Formula (I″), (I′), (I), (II), (II′), (III), (III), (IIIa), (IIIa′), (IIIb), (IIIb′), (IV), or (IV′), R¹ is hydrogen, halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, or C₁-C₆aminoalkyl; wherein each of the alkyl, alkene, alkyne, haloalkyl, and hydroxyalkyl, and aminoalkyl is optionally substituted with 1 to 4 substituents is hydrogen, halogen, —CN, —NO₂, —OH, —OR, —SH, —SR, C₁-C₆alkyl, C₁-C₆haloalkyl, C₁-C₆hydroxyalkyl, or C₁-C₆aminoalkyl; wherein each of the alkyl, haloalkyl, and hydroxyalkyl, and aminoalkyl is optionally substituted with 1 to 4 substituents independently selected from R^{e}. In some embodiments of Formula (I″), (I′), (I), (II), (II′), (III), (III′), (IIIa), (IIIa′), (IIIb), (III′), (IV), or (IV′), R¹ is hydrogen, halogen, C₁-C₆alkyl, C₁-C₆haloalkyl, C₁-C₆hydroxyalkyl, or C₁-C₆aminoalkyl. In some embodiments of Formula (I″), (I′), (I), (II), (II′), (III), (III′), (IIIa), (IIIa′), (IIIb), (III′), (IV), or (IV′), R¹ is C₁-C₆alkyl. In some embodiments of Formula (I″), (I′), (I), (II), (II′), (III), (III′), (IIIa), (IIIa′), (III), (III′), (IV), or (IV′), R¹ is C₁-C₆haloalkyl. In some embodiments of Formula (I″), (I′), (I), (II), (II′), (III), (III′), (IIIa), (IIIa′), (IIIb), (IIIb′), (IV), or (IV′), R⁸ is C₁-C₆hydroxyalkyl. In some embodiments of Formula (I″), (I′), (I), (II), (II′), (III), (III′), (IIIa), (IIIa′), (IIIb), (IIIb′), (IV), or (IV′), R¹ is halogen or hydrogen. In some embodiments of Formula (I″), (I′), (I), (II), (II′), (III), (III′), (IIIa), (IIIa′), (III), (III′), (IV), or (IV′), R⁸ is halogen. In some embodiments of Formula (I″), (I′), (I), (II), (II′), (III), (III′), (IIIa), (IIIa′), (IIIb), (IIIb′), (IV), or (IV′), R¹ is hydrogen. In some embodiments of Formula (I″), (I′), (I), (II), (II′), (III), (III′), (IIIa), (IIIa′), (IIIb), (IIIb′), (IV), or (IV′), R¹ is flourine. In some embodiments of Formula (I″), (I′), (I), (II), (II′), (III), (III), (IIIa), (IIIa′), (III), (III′), (IV), or (IV′), R¹ is chlorine.

In some embodiments of Formula (II), (II′), (III), (III′), (IIIa), (IIIa′), (IIIb), (IIIb′), (IV), or (IV′), ring G is a 5 to 6-membered heteroaryl optional substituted with 1 or 4 substituents selected from R¹⁰.

In some embodiments of Formula (II), (II′), (III), (III′), (IIIa), (IIIa′), (IIIb), (IIIb′), (IV), or (IV′), ring G is a 5-membered heteroaryl optional substituted with 1 or 2 substituents selected from R¹⁰. In some embodiments of Formula (II), (II′), (III), (III′), (IIIa), (IIIa′), (IIIb), (IIIb′), (IV), or (IV′), ring G is pyrrole, imidazole, triazole, tetrazole, thiophene, oxazole, thiazole, or thiadiazole. In some embodiments of Formula (II), (II′), (III), (III′), (IIIa), (IIIa′), (IIIb), (IIIb′), (IV), or (IV′), ring G is pyrrole, imidazole, oxazole, triazole, thiazole, or thiadiazole. In some embodiments of Formula (II), (II′), (III), (III′), (IIIa), (IIIa′), (IIIb), (IIIb′), (IV), or (IV′), ring G is pyrrole, imidazole, oxazole, or thiadiazole. In some embodiments of Formula (II), (II′) (III), (III′), (IIIa), (IIIa′), (IIIb), (IIIb′), (IV), or (IV′), ring G is pyrrole. In some embodiments of Formula (II), (II′), (III), (III′), (IIIa), (IIIa′), (IIIb), (IIIb′), (IV), or (IV′), ring G is imidazole. In some embodiments of Formula (II), (II′), (III), (III′), (IIIa), (IIIa′), (IIIb), (IIIb′), (IV), or (IV′), ring G is thiadiazole. In some embodiments of Formula (II), (II′), (III), (III′), (IIIa), (IIIa′), (IIIb), (IIIb′), (IV), or (IV′), ring G is oxazole.

In some embodiments of Formula (II), (II′), (III), (III′), (IIIa), (IIIa′), (IIIb), (IIIb′), (IV), or (IV′), ring G is:

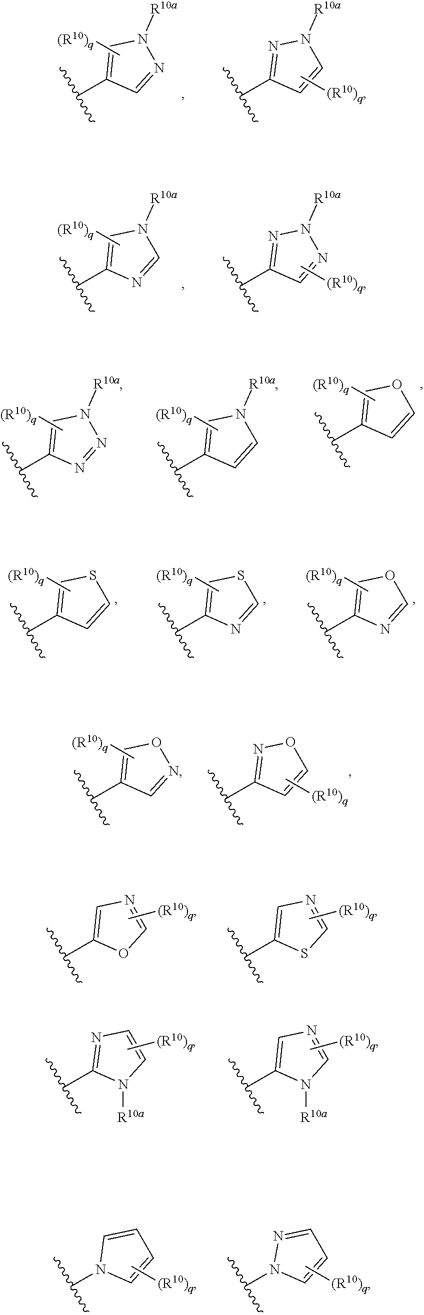

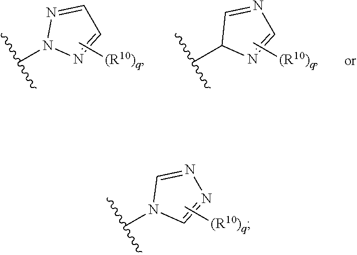

wherein,
each R¹⁰ is independently halogen, —CN, —NO₂, —OH, —OR, —NR^{c}R^{d}, C₁-C₆alkyl, C₁-C₆haloalkyl, C₁-C₆hydroxyalkyl, C₁-C₆aminoalkyl, C₃-C₆cycloalkyl, or 4 to 6-membered heterocycloalkyl, wherein each of the alkyl, haloalkyl, hydroxyalkyl, aminoalkyl, cycloalkyl and heterocycloalkyl is optionally substituted with 1 to 4-substituents independently selected from R;
each R^{10a} is independently hydrogen, C₁-C₆alkyl, C₁-C₆haloalkyl, C₃-C₆cycloalkyl, or 4 to 6-membered heterocycloalkyl, wherein each of the alkyl, haloalkyl, cycloalkyl and heterocycloalkyl is optionally substituted with 1 to 4-substituents independently selected from R^{e}; and
q is 0, 1, 2, or 3.

In some embodiments of Formula (II), (II′), (III), (III′), (IIIa), (IIIa′), (IIIb), (IIIb′), (IV), or (IV′): each R¹⁰ is independently halogen, —CN, —NO2, —OH, —ORa, C1 C6alkyl, C1 C6haloalkyl, C3-C6cycloalkyl, or 4 to 6-membered heterocycloalkyl; each R^{10a} is independently hydrogen, C₁-C₆alkyl, C₁-C₆haloalkyl, C₃-C₆cycloalkyl, or 4 to 6-membered heterocycloalkyl; and p is 0, 1, or 2.

In some embodiments of Formula (II), (II′), (III), (III′), (IIIa), (IIIa′), (IIIb), (IIIb′), (IV), or (IV′),

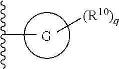

is:

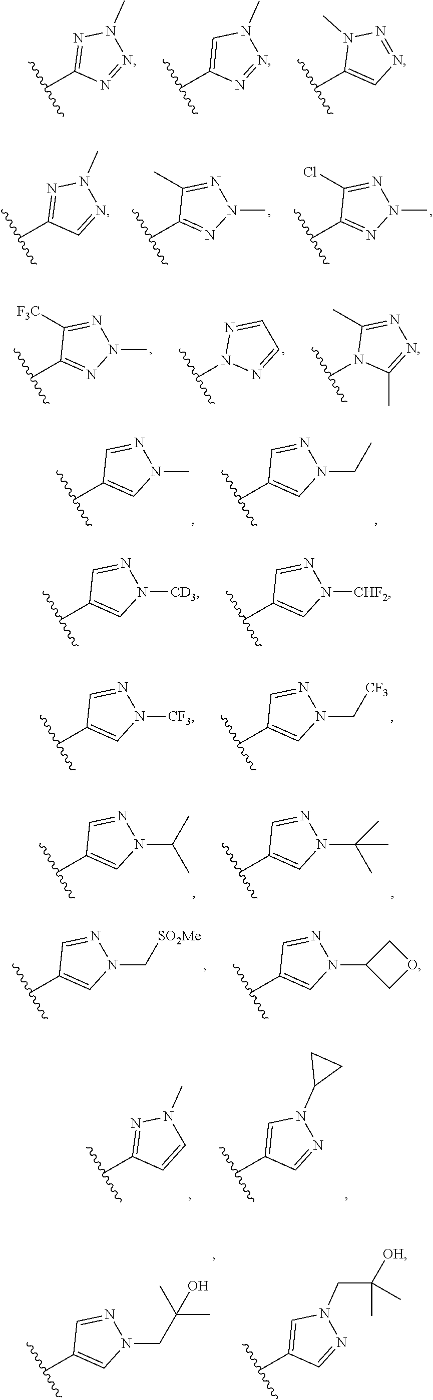

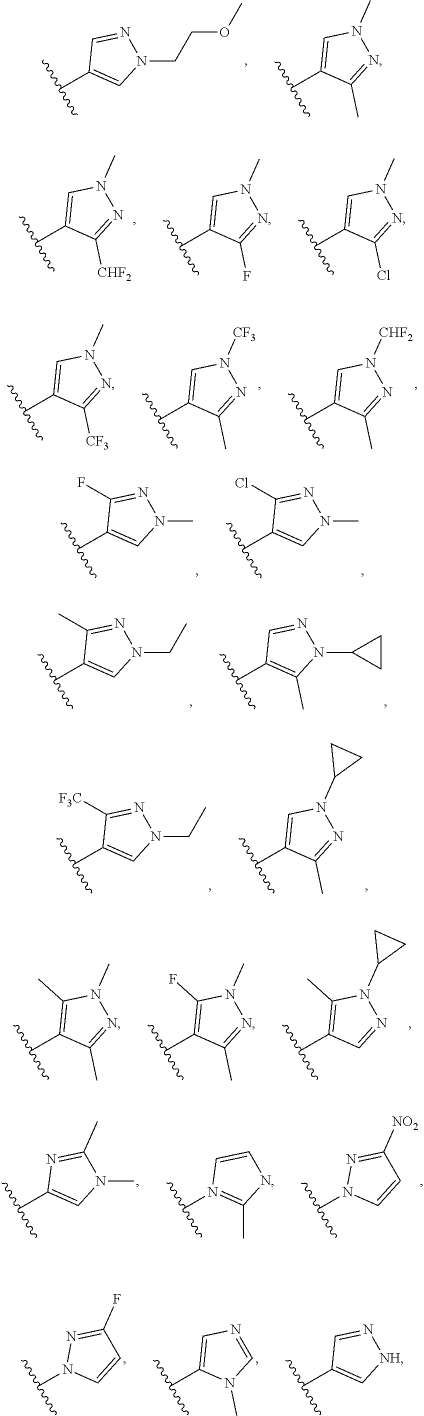

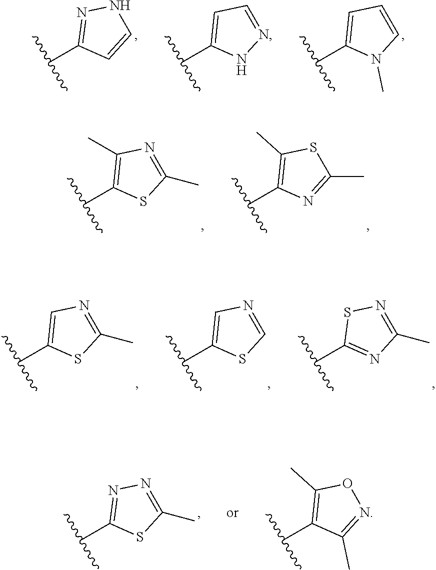

In some embodiments of Formula (11), (II′), (111), (111′), (IIa), (IIa′), (IIIb), (IIIb′), (IV), or (IV′),

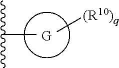

is:

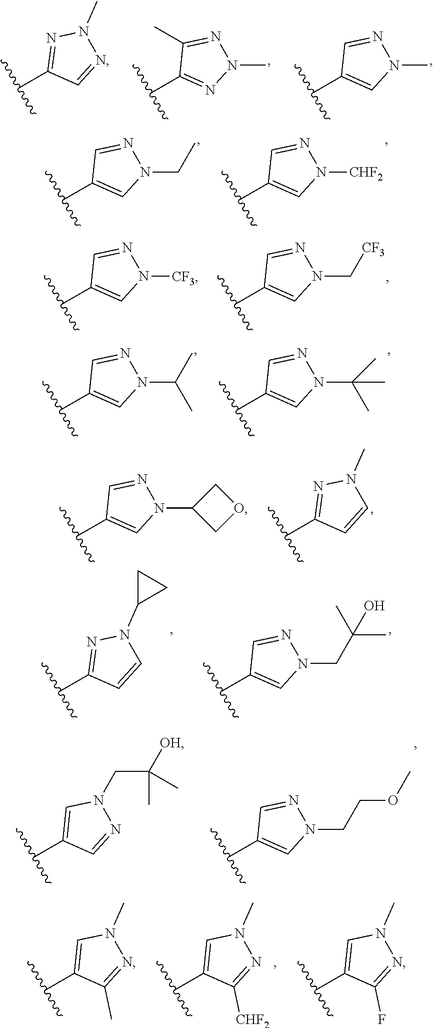

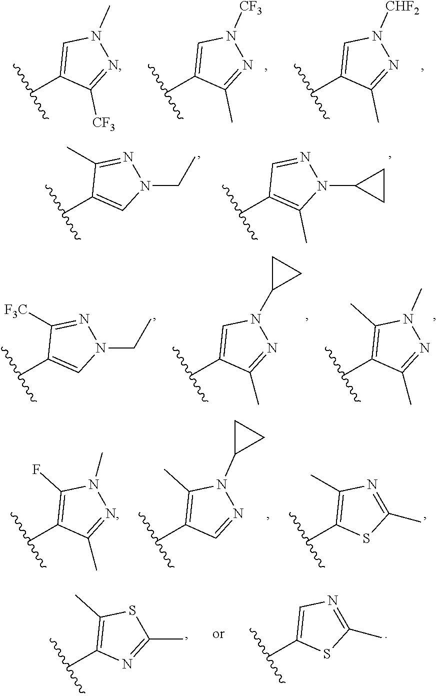

In some embodiments of Formula (II), (II′), (III), (III′), (IIIa), (IIIa′), (IIIb), (IIIb′), (IV), or (IV′),

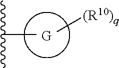

is:

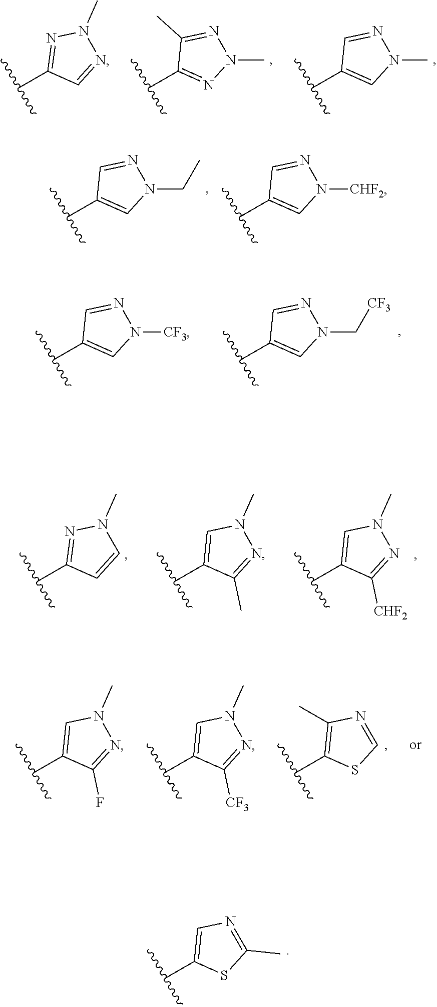

In some embodiments of Formula (II), (II′), (III), (III′), (IIIa), (IIIa′), (IIIb), (IIIb′), (IV), or (IV′),

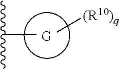

is:

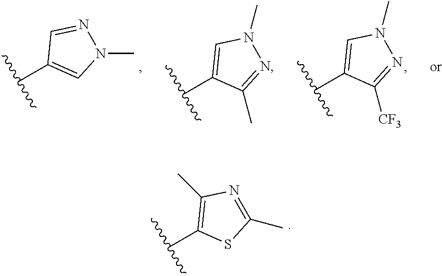

In some embodiments of Formula (II), (II′), (III), (III′), (IIIa), (IIIa′), (IIIb), (IIIb′), (IV), or (IV′),

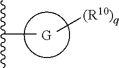

is:

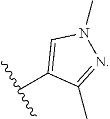 [SMILES: *c1cn(C)nc1C]

In some embodiments of Formula (II), (II′), (III), (III′), (IIIa), (IIa′), (IIIb), (IIIb′), (IV), or (IV′),

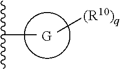

is:

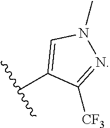 [SMILES: *c1cn(C)nc1C(F)(F)F]

In some embodiments of Formula (II), (II′), (III), (III′), (IIIa), (IIa′), (IIIb), (IIIb′), (IV), or (IV′),

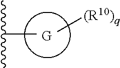

is:

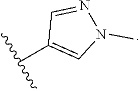 [SMILES: *c1cnn(C)c1]

In some embodiments of Formula (II), (II′), (III), (III′), (IIIa), (IIIa′), (IIIb), (IIIb′), (IV), or (IV′),

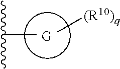

is:

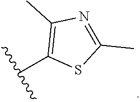 [SMILES: *c1sc(C)nc1C]

In some embodiments of Formula (II), (II′), (III), (III′), (IIIa), (IIIa′), (IIIb), (IIIb′), (IV), or (IV′), ring G is a 6-membered heteroaryl optional substituted with 1 to 4 substituents selected from R¹⁰.

In some embodiments of Formula (II), (II′), (III), (III′), (IIIa), (IIIa′), (IIIb), (IIIb′), (IV), or (IV′), ring G is pyridine, pyrimidine, or triazine. In some embodiments of Formula (II), (II′), (III), (III′), (IIIa), (IIIa′), (IIIb), (IIIb′), (IV), or (IV′), ring G is pyridine. In some embodiments of Formula (II), (II′), (III), (III′), (IIIa), (IIIa′), (IIIb), (IIIb′), (IV), or (IV′), ring G is pyrimidine. In some embodiments of Formula (II), (II′), (III), (III′), (IIIa), (IIIa′), (IIIb), (IIIb′), (IV), or (IV′), ring G is triazine.

In some embodiments of Formula (II), (II′), (III), (III′), (IIIa), (IIIa′), (IIIb), (IIIb′), (IV), or (IV′), ring G is:

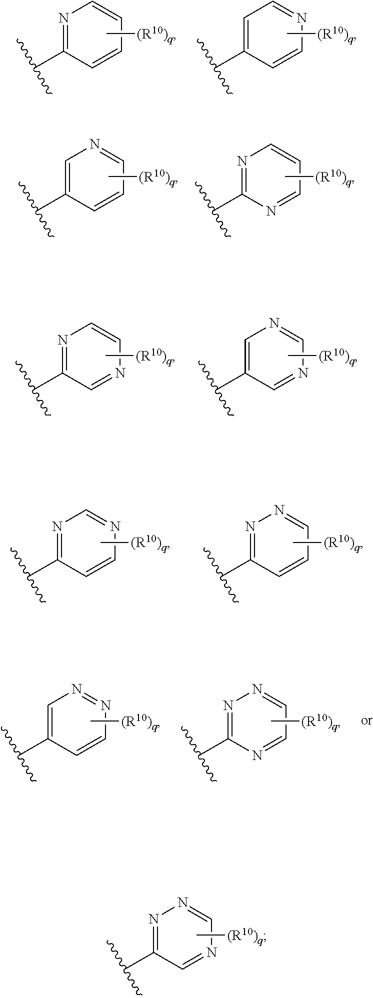

wherein,
each R¹⁰ is independently halogen, —CN, —NO₂, —OH, —OR^{a}, —NR^{c}R^{d}, C₁-C₆alkyl, C₁-C₆haloalkyl, C₁-C₆hydroxyalkyl, C₁-C₆aminoalkyl, C₃-C₆cycloalkyl, or 4 to 6-membered heterocycloalkyl, wherein each of the alkyl, haloalkyl, hydroxyalkyl, aminoalkyl, cycloalkyl and heterocycloalkyl is optionally substituted with 1 to 4-substituents independently selected from R; and
q is 0, 1, 2, or 3.

In some embodiments of Formula (II), (II′), (III), (III′), (IIIa), (IIIa′), (IIIb), (IIIb′), (IV), or (IV′),

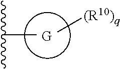

is:

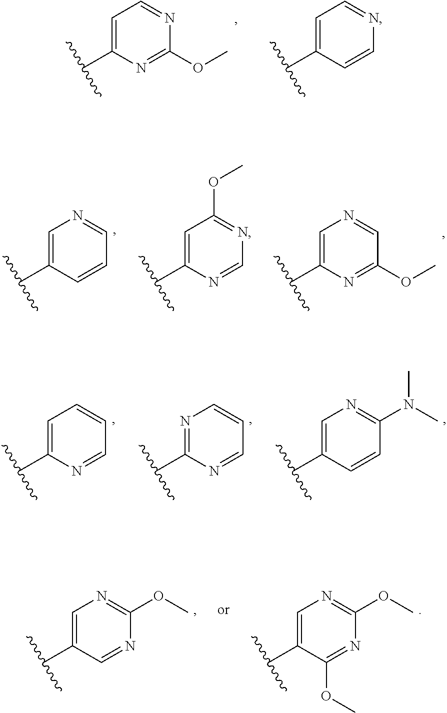

In some embodiments of Formula (II), (II′), (III), (III′), (IIIa), (IIIa′), (IIIb), (IIIb′), (IV), or (IV′), ring G is an 8 to 10-membered bicyclic heteroaryl comprising 1 to 3 heteroatoms selected from N, O, and S; and optional substituted with 1 to 4 substituents selected from R¹⁰. In some embodiments of Formula (II), (II′), (III), (III′), (IIIa), (IIIa′), (IIIb), (IIIb′), (IV), or (IV′), ring G is a 10-membered bicyclic heteroaryl comprising 1 to 3 heteroatoms selected from N, O, and S; and optional substituted with 1 to 4 substituents selected from R¹⁰. In some embodiments of Formula (II), (II′), (III), (III′), (IIIa), (IIIa′), (IIIb), (IIIb′), (IV), or (IV′), ring G is an 8-membered bicyclic heteroaryl comprising 1 to 3 heteroatoms selected from N, O, and S; and optional substituted with 1 to 4 substituents selected from R¹⁰. In some embodiments of Formula (II), (II′), (III), (III′), (IIIa), (IIIa′), (IIIb), (IIIb′), (IV), or (IV′), ring G is an 8-membered bicyclic heteroaryl comprising 1 to 3 heteroatoms selected from N; and optional substituted with 1 to 4 substituents selected from R¹⁰.

In some embodiments of Formula (II), (II′), (III), (III′), (IIIa), (IIIa′), (IIIb), (IIIb′), (IV), or (IV′), ring G is imidazo[1,2-a]pyrazine, imidazo[1,2-a]pyrimidine, pyrazolo[1,5-a]pyridine, pyrazolo[1,5-a]pyrimidine, or 2-methyl-2H-indazole.

In some embodiments of Formula (II), (II′), (III), (III′), (IIIa), (IIIa′), (IIIb), (IIIb′), (IV), or (IV′),

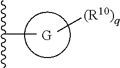

is

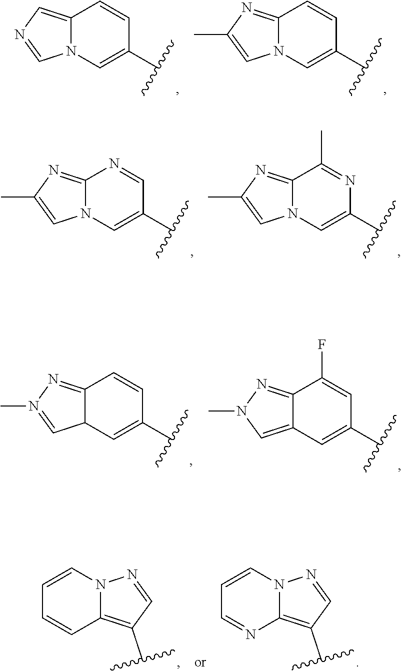

In some embodiments of Formula (I′), (I), (II), (II′), (III), (III′), (IIIa), (IIIa′), (IIIb), (IIIb′), (IV), or (IV′), each R¹⁰ is independently halogen, —CN, —NO₂, —OH, —OR, —NR^{c}R^{d}, C₁-C₆alkyl, C₁-C₆haloalkyl, C₁-C₆heteroalkyl, C₁-C₆hydroxyalkyl, C₁-C₆aminoalkyl, C₃-C₆cycloalkyl, or 4 to 6-membered heterocycloalkyl, wherein each of the alkyl, haloalkyl, hydroxyalkyl, aminoalkyl, cycloalkyl and heterocycloalkyl is optionally substituted with 1 to 4-substituents independently selected from R*. In some embodiments of Formula (I′), (I), (II), (II′), (III), (III′), (IIIa), (IIIa′), (IIIb), (IIIb′), (IV), or (IV′), each R¹⁰ is independently —CN, —NO₂, —OH, —OR^{a}, or —NR^{c}R^{d}. In some embodiments of Formula (I′), (I), (II), (II′), (III), (III′), (IIIa), (IIIa′), (IIIb), (IIIb′), (IV), or (IV′), each R¹⁰ is independently —OH or —OR^{a}. In some embodiments of Formula (I′), (I), (II), (II′), (III), (III′), (IIIa), (IIIa′), (IIIb), (IIIb′), (IV), or (IV′), each R¹⁰ is independently —OH or —OMe. In some embodiments of Formula (I′), (I), (II), (II′), (III), (III′), (IIIa), (IIIa′), (IIIb), (IIIb′), (IV), or (IV′), each R¹⁰ is independently halogen, C₁-C₆alkyl, C₁-C₆haloalkyl, C₁-C₆hydroxyalkyl, C₁-C₆aminoalkyl, or C₃-C₆cycloalkyl. In some embodiments of Formula (I′), (I), (II), (II′), (III), (III′), (IIIa), (IIIa′), (IIIb), (IIIb′), (IV), or (IV′), each R¹⁰ is independently halogen, C₁-C₆alkyl, C₁-C₆haloalkyl, C₁-C₆hydroxyalkyl. In some embodiments of Formula (I′), (I), (II), (II′), (III), (III′), (IIIa), (IIa′), (IIIb), (IIIb′), (IV), or (IV′), each R¹⁰ is independently halogen, C₁-C₆alkyl, C₁-C₆haloalkyl. In some embodiments of Formula (I′), (I), (II), (II′), (III), (III′), (IIIa), (IIIa′), (IIIb), (IIIb′), (IV), or (IV′), each R¹⁰ is independently C₁-C₆haloalkyl. In some embodiments of Formula (I′), (I), (II), (II′), (III), (III′), (IIIa), (IIIa′), (IIb), (IIb′), (IV), or (IV′), each R¹⁰ is independently —CF₃, —CHF₂, —CH₂CF₃, or —CH₂CHF₂. In some embodiments of Formula (I′), (I), (II), (II′), (III), (III′), (IIIa), (IIIa′), (IIb), (IIb′), (IV), or (IV′), each R¹⁰ is independently —CF₃ or —CHF₂. In some embodiments of Formula (I′), (I), (II), (II′), (III), (III′), (IIIa), (IIIa′), (IIIb), (IIIb′), (IV), or (IV′), each R¹⁰ is independently C₁-C₆alkyl. In some embodiments of Formula (I′), (I), (II), (II′), (III), (III′), (IIIa), (IIIa′), (IIIb), (IIIb′), (IV), or (IV′), each R¹⁰ is independently methyl, ethyl, isopropyl, or tert-butyl. In some embodiments of Formula (I′), (I), (II), (II′), (III), (III′), (IIIa), (IIIa′), (IIIb), (IIIb), (IV), or (IV′), each R¹⁰ is independently methyl. In some embodiments of Formula (I′), (I), (II), (II′), (III), (III′), (IIIa), (IIIa′), (IIIb), (IIb′), (IV), or (IV′), each R¹⁰ is independently halogen. In some embodiments of Formula (I′), (I), (II), (II′), (III), (III′), (IIIa), (IIIa′), (IIIb), (IIIb′), (IV), or (IV′), each R¹⁰ is independently fluorine or chlorine. In some embodiments of Formula (I′), (I), (II), (II′), (III), (III′), (IIIa), (IIIa′), (IIIb), (IIIb′), (IV), or (IV′), each R¹⁰ is independently fluorine. In some embodiments of Formula (I′), (I), (II), (II′), (III), (III′), (IIIa), (IIIa′), (IIIb), (IIIb′), (IV), or (IV′), each R¹⁰ is independently chlorine.

In some embodiments of Formula (I′), (I), (II), (II′), (III), (III′), (IIIa), (IIIa′), (IIIb), (IIIb′), (IV), or (IV′), each R¹⁰ is independently —F, —Cl, —CN, methyl, ethyl, —OCH₃, —CF₃, —CHF₂, or —CH₂F. In some embodiments of Formula (I′), (I), (II), (II′), (III), (III′), (IIIa), (IIIa′), (IIb), (IIb′), (IV), or (IV′), each R¹⁰ is independently —F, —C₁, methyl, —CF₃, or —CHF₂.

In some embodiments of Formula (I′), (I), (II), (II′), (III), (III′), (IIIa), (IIIa′), (IIIb), (IIIb′), (IV), or (IV′), two R¹⁰ are taken together to form an oxo.

In some embodiments of Formula (II), (II′), (III), (III′), (IIIa), (IIIa′), (IIb), (IIb′), (IV), or (IV′), each R^{10a} is independently hydrogen, C₁-C₆alkyl, C₁-C₆haloalkyl, C₃-C₆cycloalkyl, or 4 to 6-membered heterocycloalkyl. In some embodiments of Formula (II), (II′), (III), (III′), (lIIa), (IIIa′), (IIIb), (IIIb′), (IV), or (IV′), each R^{10a} is independently C₃-C₆cycloalkyl. In some embodiments of Formula (II), (II′), (III), (III′), (IIIa), (IIIa′), (IIIb), (IIIb′), (IV), or (IV′), each R^{10a} is independently cyclopropyl, cyclobutyl, cyclopentyl, or cyclohexyl. In some embodiments of Formula (II), (II′), (III), (III′), (IIIa), (IIIa′), (IIb), (IIb′), (IV), or (IV′), each R^{10a} is independently cyclopropyl. In some embodiments of Formula (II), (II′), (III), (III′), (IIIa), (IIIa′), (IIIb), (IIIb′), (IV), or (IV′), each R^{10a} is independently cyclopropyl. In some embodiments of Formula (II), (II′), (III), (III′), (IIIa), (IIIa′), (IIb), (IIb′), (IV), or (IV′), each R^{10a} is independently 4 to 6-membered heterocycloalkyl. In some embodiments of Formula (II), (II′), (III), (III′), (IIIa), (IIIa′), (IIIb), (IIIb′), (IV), or (IV′), each R^{10a} is independently 4-membered heterocycloalkyl. In some embodiments of Formula (II), (II′), (III), (III′), (IIIa), (IIIa′), (IIIb), (IIIb′), (IV), or (IV′), each R^{10a} is independently 5-membered heterocycloalkyl. In some embodiments of Formula (II), (II′), (III), (III′), (IIIa), (IIIa′), (IIb), (IIIb′), (IV), or (IV′), each R^{10a} is independently 6-membered heterocycloalkyl. In some embodiments of Formula (II), (II′), (III), (III′), (IIIa), (IIIa′), (IIIb), (IIIb′), (IV), or (IV′), each R^{10a} is independently oxatanyl. In some embodiments of Formula (II), (II′), (III), (III′), (IIIa), (IIIa′), (IIIb), (IIIb′), (IV), or (IV′), each R^{10a} is independently C₁-C₆haloalkyl. In some embodiments of Formula (II), (II′), (III), (III′), (IIIa), (IIIa′), (IIIb), (IIIb′), (IV), or (IV′), each R^{10a} is independently —CF₃, —CHF₂, —CH₂CF₃, or —CH₂CHF₂. In some embodiments of Formula (II), (II′), (III), (III′), (IIIa), (IIIa′), (IIIb), (IIIb′), (IV), or (IV′), each R^{10a} is independently C₁-C₆alkyl. In some embodiments of Formula (II), (II′), (III), (III′), (IIIa), (IIIa′), (IIIb), (IIIb′), (IV), or (IV′), each R^{10a} is independently methyl, ethyl, isopropyl, or tert-butyl. In some embodiments of Formula (II), (II′), (III), (III′), (IIIa), (IIIa′), (IIIb), (IIIb′), (IV), or (IV′), each R^{10a} is independently methyl. In some embodiments of Formula (II), (II′), (III), (III′), (IIIa), (IIIa′), (IIIb), (IIIb′), (IV), or (IV′), each R^{10a} is independently hydrogen.

In some embodiments of Formula (II), (II′), (III), (III′), (IIIa), (IIIa′), (IIIb), (IIIb′), (IV), or (IV′), each R^{10a} is independently hydrogen, methyl, ethyl, isopropyl, tert-butyl, —CD₃, —CF₃, CHF₂, CH₂F, —CH₂CF₃, —CH₂SO₂CH₃, cyclopropyl, oxetanyl,

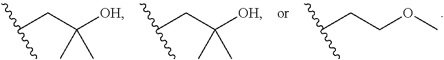

In some embodiments of Formula (I′), (I), (II), (II′), (III), (III′), (IIIa), (IIIa′), (IIIb), (IIIb′), (IV), or (IV′), p is an integer from 1-9. In some embodiments of Formula (I′), (I), (II), (II′), (III), (III′), (IIIa), (IIIa′), (IIIb), (IIIb′), (IV), or (IV′), p is an integer from 1-8. In some embodiments of Formula (I′), (I), (II), (II′), (III), (III′), (IIIa), (IIIa′), (IIIb), (IIIb′), (IV), or (IV′), p is an integer from 1-6. In some embodiments of Formula (I′), (I), (II), (II′), (III), (III′), (IIIa), (IIIa′), (IIIb), (IIIb′), (IV), or (IV′), p is an integer from 1-4. In some embodiments of Formula (I′), (I), (II), (II′), (III), (III′, (IIIa), (IIIa′), (IIIb), (IIIb′), (IV), or (IV′), p is 1, 2, 3, 4, 5, 6, 7, or 8. In some embodiments of Formula (I′), (I), (II), (II′), (III), (III′), (IIIa), (IIIa′), (IIIb), (IIIb′), (IV), or (IV′), p is 1, 2, 3, 4, 5, or 6. In some embodiments of Formula (I′), (I), (II), (II′), (III), (III′), (IIIa), (IIIa′), (IIIb), (IIIb′), (IV), or (IV′), p is 1, 2, 3, or 4. In some embodiments of Formula (I′), (I), (II), (II′), (III), (III′), (IIIa), (IIIa′), (IIIb), (IIIb′), (IV), or (IV′), p is 1, 2, 3, 4, 5, or 6. In some embodiments of Formula (I′), (I), (II), (II′), (III), (III′), (IIIa), (IIIa′), (IIIb), (IIIb′), (IV), or (IV′), p is 1, 2, or 3. In some embodiments of Formula (I′), (I), (II), (II′), (III), (III′), (IIIa), (IIIa′), (IIIb), (IIIb′), (IV), or (IV′), p is 1, 2, 3, 4, 5, or 6. In some embodiments of Formula (I′), (I), (II), (II′), (III), (III′), (IIIa), (IIIa′), (IIb), (IIb′), (IV), or (IV′), p is 1 or 2. In some embodiments of Formula (I′), (I), (II), (II′), (III), (III′), (IIIa), (IIIa′), (IIIb), (IIIb′), (IV), or (IV′), p is 1, 2, 3, 4, 5, or 6. In some embodiments of Formula (I′), (I), (II), (II′), (II), (III′), (IIIa), (IIIa′), (IIIb), (IIIb′), (IV), or (IV′), p is 2. In some embodiments of Formula (I′), (I), (II), (II′), (III), (III), (IIIa), (IIIa′), (IIIb), (IIIb′), (IV), or (IV′), p is 1.

In some embodiments of Formula (II), (II′), (III), (III′), (IIIa), (IIIa′), (IIIb), (IIIb′), (IV), or (IV′), q is 1, 2, or 3. In some embodiments of Formula (II), (II′), (III), (III′), (IIIa), (IIIa′), (IIIb), (IIIb′), (IV), or (IV′), q is 3. In some embodiments of Formula (II), (II′), (III), (III′), (IIIa), (IIIa′), (IIIb), (IIb′), (IV), or (IV′), q is 1 or 2. In some embodiments of Formula (II), (II′), (III), (III′), (IIIa), (IIIa′), (IIIb), (IIIb′), (IV), or (IV′), q is 2. In some embodiments of Formula (II), (II′), (III), (III′), (IIIa), (IIIa′), (IIIb), (IIIb′), (IV), or (IV′), q is 1. In some embodiments of Formula (II), (II′), (III), (III′), (IIIa), (IIIa′), (IIIb), (IIIb′), (IV), or (IV′), q is 0.

In some embodiments of the compound disclosed herein each Rₐ is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e}. In some embodiments of a compound disclosed herein, each Rₐ is independently C₁-C₆alkyl, C₁-C₆haloalkyl, or cycloalkyl, heterocycloalkyl. In some embodiments of a compound disclosed herein, each Rₐ is independently C₁-C₆alkyl or C₁-C₆haloalkyl. In some embodiments of a compound disclosed herein, each Rₐ is independently C₁-C₆alkyl. In some embodiments of a compound disclosed herein, each Rₐ is independently methyl, ethyl, isopropyl, tert-butyl, —OCH₃, —CF₃, or —CHF₂, In some embodiments of a compound disclosed herein, each Rₐ is independently methyl, ethyl, isopropyl or tertbutyl.

In some embodiments of the compound disclosed herein, each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e}. In some embodiments of a compound disclosed herein, each R^{b} is independently hydrogen, C₁-C₆alkyl, C₁-C₆haloalkyl, or cycloalkyl, heterocycloalkyl. In some embodiments of a compound disclosed herein, each R¹ is independently hydrogen, C₁-C₆alkyl or C₁-C₆haloalkyl. In some embodiments of a compound disclosed herein, each R^{b} is independently hydrogen or C₁-C₆alkyl. In some embodiments of a compound disclosed herein, each R¹ is hydrogen. In some embodiments of a compound disclosed herein, each R¹ is independently C₁-C₆alkyl. In some embodiments of a compound disclosed herein, each R^{b} is independently hydrogen, methyl, ethyl, isopropyl, tert-butyl, —OCH₃, —CF₃, or —CHF₂, In some embodiments of a compound disclosed herein, each R^{b} is independently hydrogen, methyl, ethyl, isopropyl, or tert-butyl. In some embodiments of a compound disclosed herein, each R^{b} is independently hydrogen or methyl.

In some embodiments of the compound disclosed herein, R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e}. In some embodiments of a compound disclosed herein, each R^{c} and R^{d} are independently hydrogen, C₁-C₆alkyl or C₁-C₆haloalkyl. In some embodiments of a compound disclosed herein, each R^{c} and R^{d} are independently hydrogen or C₁-C₆alkyl. In some embodiments of a compound disclosed herein, each R^{c} and R^{d} are hydrogen. In some embodiments of a compound disclosed herein, each R^{c} and R^{d} are independently C₁-C₆alkyl. In some embodiments of a compound disclosed herein, each R^{c} and R^{d} are independently hydrogen methyl, ethyl, isopropyl, tert-butyl, —OCH₃, —CF₃, or —CHF₂, In some embodiments of a compound disclosed herein, each R^{c} and R^{d} are independently hydrogen, methyl, ethyl, isopropyl, or tert-butyl. In some embodiments of a compound disclosed herein, each R^{c} and R^{d} are independently hydrogen or methyl. In some embodiments of a compound disclosed herein, each R^{c} and R^{d} are methyl.

In some embodiments of a compound disclosed herein, each R^{e} is independently halogen, oxo, —CN, —OH, —S(═O)CH₃, —S(═O)₂CH₃, —CH₂S(═O)₂CH₃, —S(═O)₂NH₂, —CH₂S(═O)₂CH₃, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6-membered heterocycloalkyl. In some embodiments of a compound disclosed herein, each R¹ is independently halogen, oxo, —CN, —OH, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl. In some embodiments of a compound disclosed herein, each R¹ is independently halogen, oxo, —CN, —OH, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁₋₆alkyl, —N(C₁₋₆alkyl)₂, —C(═O)C₁₋₆alkyl, —C(═O)OH, —C(═O)OC₁-C₆alkyl, —NHC(═O)C₁-C₆alkyl, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl. In some embodiments, R^{e} is —NHC(═O)C₁₋₃alkyl such as —NHC(═O)CH₃. In some embodiments of a compound disclosed herein, each R^{e} is independently halogen, —CN, —OH, or C₁-C₆alkyl. In some embodiments of a compound disclosed herein, each R^{e} is independently halogen, —OH, or C₁-C₆alkyl. In some embodiments of a compound disclosed herein, each R^{e} is independently halogen or C₁-C₆alkyl. In some embodiments of a compound disclosed herein, each R^{e} is independently halogen. In some embodiments of a compound disclosed herein, each R^{e} is independently —C₁ or —F. In some embodiments of a compound disclosed herein, each R^{e} is independently halogen, —CN, —OH, —S(═O)CH₃, —S(═O)₂CH₃, —CH₂S(═O)₂CH₃, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₃-C₆cycloalkyl, or 4 to 6-membered heterocycloalkyl. In some embodiments, each R is independently —F, —C₁, —OH, —CH₂S(═O)₂CH₃, methyl, ethyl, isopropyl, tertbutyl, —OCH₃, cyclopropyl, cyclobutyl, cyclopentyl, cyclohexyl, or azetidine, oxytane, piperidine, piperazine, or morpholine. In some embodiments, each R is independently —F, —OH, —CF₃, —CHF₂, —OCH₃, or methyl. In some embodiments, each R^{e} is independently —F, —CF₃, —CHF₂, or methyl. In some embodiments, each R is methyl.

In some embodiments of a compound disclosed herein, one or more of R¹, R², R³, R⁴, R⁵, R⁶, R⁷, R⁸, R⁹, R¹⁰, R^{10a}, R^{a}, R^{b}, R^{c}, R^{d}, and R^{e} groups comprise deuterium at a percentage higher than the natural abundance of deuterium.

In some embodiments of a compound disclosed herein, one or more ¹H are replaced with one or more deuteriums in one or more of the following groups R¹, R², R³, R⁴, R⁵, R⁶, R⁷, R⁸, R⁹, R¹⁰, R^{10a}, R^{a}, R^{b}, R^{c}, R^{d}, and R^{e}.

In some embodiments of a compound disclosed herein, the abundance of deuterium in each of R¹, R², R³, R⁴, R⁵, R⁶, R⁷, R⁸, R⁹, R¹⁰, R^{10a}, R^{a}, R^{b}, R^{c}, R^{d}, and R^{e} is independently at least 1%, at least 10%, at least 20%, at least 30%, at least 40%, at least 50%, at least 60%, at least 70%, at least 80%, at least 90%, or 100% by molar.

Any combination of the groups described above for the various variables is contemplated herein. Throughout the specification, groups and substituents thereof are chosen by one skilled in the field to provide stable moieties and compounds.

In some embodiments the compound disclosed herein, or a pharmaceutically acceptable salt or solvate thereof, is one of the compounds in Table 3A, Table 3B, or Table 3C.

TABLE 3A

| No. | Chemical Structure | Name |
|---|---|---|
| 1 | 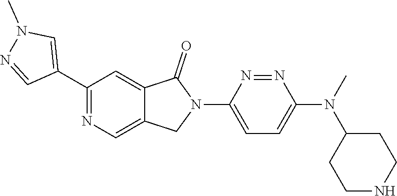 [SMILES: CN(c1ccc(N2Cc3cnc(-c4cnn(C)c4)cc3C2=O)nn1)C1CCNCC1] | 2-(6-(methyl(piperidin-4- yl)amino)pyridazin-3-yl)-6-(1- methyl-1H-pyrazol-4-yl)-2,3- dihydro-1H-pyrrolo[3,4-c]pyridin- 1-one |
| 2 | 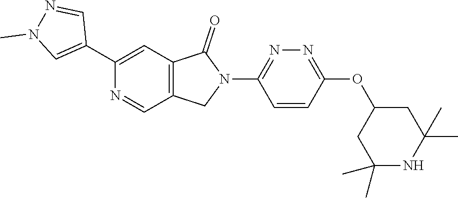 | 6-(1-methyl-1H-pyrazol-4-yl)-2- (6-((2,2,6,6-tetramethylpiperidin- 4-yl)oxy)pyridazin-3-yl)-2,3- dihydro-1H-pyrrolo[3,4-c]pyridin- 1-one |
| 3 | 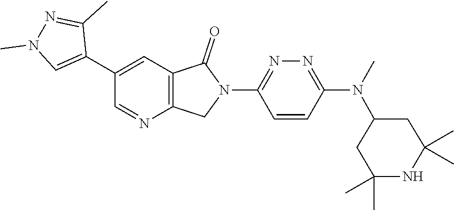 | 2-(6-(methyl(2,2,6,6- tetramethylpiperidin-4- yl)amino)pyridazin-3-yl)-6-(1- methyl-1H-pyrazol-4-yl)-2,3- dihydro-1H-pyrrolo[3,4-c]pyridin- 1-one |
| 6 | 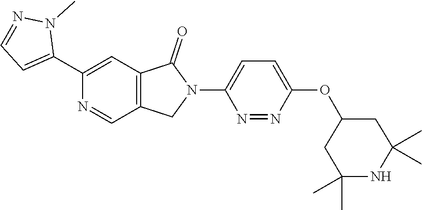 [SMILES: Cn1nccc1-c1cc2c(cn1)CN(c1ccc(OC3CC(C)(C)NC(C)(C)C3)nn1)C2=O] | 6-(1-methyl-1H-pyrazol-5-yl)-2- (6-((2,2,6,6-tetramethylpiperidin- 4-yl)oxy)pyridazin-3-yl)-2,3- dihydro-1H-pyrrolo[3,4-c]pyridin- 1-one |
| 7 | 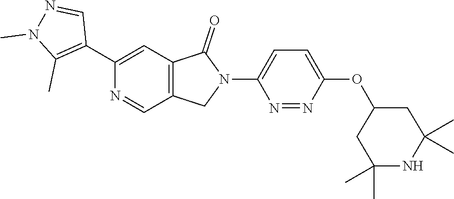 | 6-(1,5-dimethyl-1H-pyrazol-4-yl)- 2-(6-((2,2,6,6- tetramethylpiperidin-4- yl)oxy)pyridazin-3-yl)-2,3- dihydro-1H-pyrrolo[3,4-c]pyridin- 1-one |
| 8 | 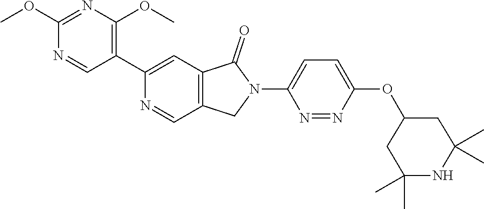 | 6-(2,4-dimethoxypyrimidin-5-yl)- 2-(6-((2,2,6,6- tetramethylpiperidin-4- yl)oxy)pyridazin-3-yl)-2,3- dihydro-1H-pyrrolo[3,4-c]pyridin- 1-one |
| 9 | 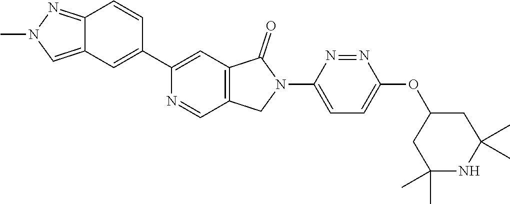 | 6-(2-methyl-2H-indazol-5-yl)-2- (6-((2,2,6,6-tetramethylpiperidin- 4-yl)oxy)pyridazin-3-yl)-2,3- dihydro-1H-pyrrolo[3,4-c]pyridin- 1-one |
| 10 | 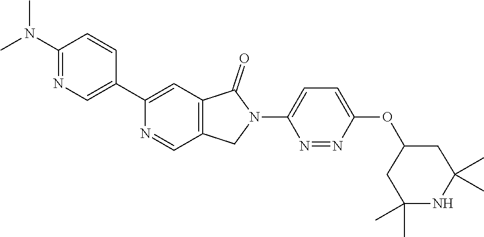 | 6-(6-(dimethylamino)pyridin-3- yl)-2-(6-((2,2,6,6- tetramethylpiperidin-4- yl)oxy)pyridazin-3-yl)-2,3- dihydro-1H-pyrrolo[3,4-c]pyridin- 1-one |
| 15 | 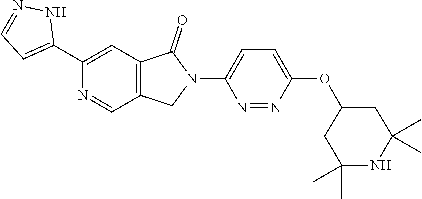 [SMILES: CC1(C)CC(Oc2ccc(N3Cc4cnc(-c5ccn[nH]5)cc4C3=O)nn2)CC(C)(C)N1] | 6-(1H-pyrazol-5-yl)-2-(6-((2,2,6,6- tetramethylpiperidin-4- yl)oxy)pyridazin-3-yl)-2,3- dihydro-1H-pyrrolo[3,4-c]pyridin- 1-one |
| 17 | 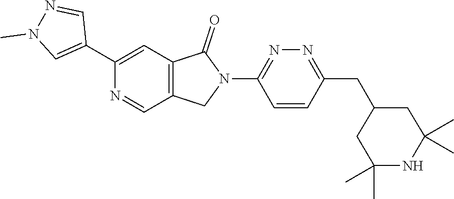 | 6-(1-methyl-1H-pyrazol-4-yl)-2- (6-((2,2,6,6-tetramethylpiperidin- 4-yl)methyl)pyridazin-3-yl)-2,3- dihydro-1H-pyrrolo[3,4-c]pyridin- 1-one |
| 18 | 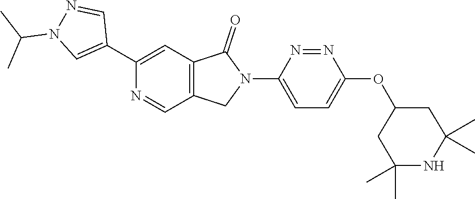 [SMILES: CC(C)n1cc(-c2cc3c(cn2)CN(c2ccc(OC4CC(C)(C)NC(C)(C)C4)nn2)C3=O)cn1] | 6-(1-isopropyl-1H-pyrazol-4-yl)-2- (6-((2,2,6,6-tetramethylpiperidin- 4-yl)oxy)pyridazin-3-yl)-2,3- dihydro-1H-pyrrolo[3,4-c]pyridin- 1-one |
| 21 | 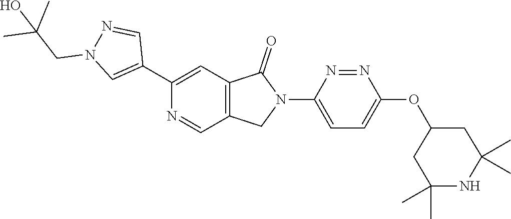 [SMILES: CC(C)(O)Cn1cc(-c2cc3c(cn2)CN(c2ccc(OC4CC(C)(C)NC(C)(C)C4)nn2)C3=O)cn1] | 6-(1-(2-hydroxy-2-methylpropyl)- 1H-pyrazol-4-yl)-2-(6-((2,2,6,6- tetramethylpiperidin-4- yl)oxy)pyridazin-3-yl)-2,3- dihydro-1H-pyrrolo[3,4-c]pyridin- 1-one |
| 23 | 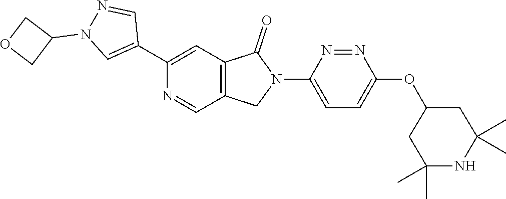 [SMILES: CC1(C)CC(Oc2ccc(N3Cc4cnc(-c5cnn(C6COC6)c5)cc4C3=O)nn2)CC(C)(C)N1] | 6-(1-(oxetan-3-yl)-1H-pyrazol-4- yl)-2-(6-((2,2,6,6- tetramethylpiperidin-4- yl)oxy)pyridazin-3-yl)-2,3- dihydro-1H-pyrrolo[3,4-c]pyridin- 1-one |
| 24 | 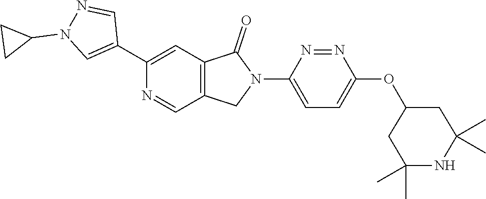 [SMILES: CC1(C)CC(Oc2ccc(N3Cc4cnc(-c5cnn(C6CC6)c5)cc4C3=O)nn2)CC(C)(C)N1] | 6-(1-cyclopropyl-1H-pyrazol-4- yl)-2-(6-((2,2,6,6- tetramethylpiperidin-4- yl)oxy)pyridazin-3-yl)-2,3- dihydro-1H-pyrrolo[3,4-c]pyridin- 1-one |
| 26 | 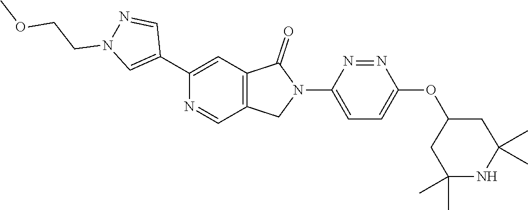 | 6-(1-(2-methoxyethyl)-1H- pyrazol-4-yl)-2-(6-((2,2,6,6- tetramethylpiperidin-4- yl)oxy)pyridazin-3-yl)-2,3- dihydro-1H-pyrrolo[3,4-c]pyridin- 1-one |
| 28 | 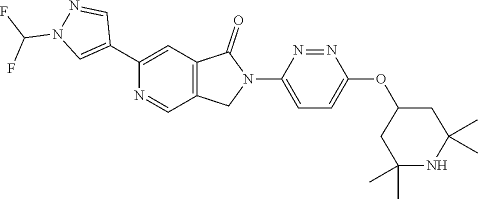 [SMILES: CC1(C)CC(Oc2ccc(N3Cc4cnc(-c5cnn(C(F)F)c5)cc4C3=O)nn2)CC(C)(C)N1] | 6-(1-(difluoromethyl)-1H-pyrazol- 4-yl)-2-(6-((2,2,6,6- tetramethylpiperidin-4- yl)oxy)pyridazin-3-yl)-2,3- dihydro-1H-pyrrolo[3,4-c]pyridin- 1-one |
| 29 | 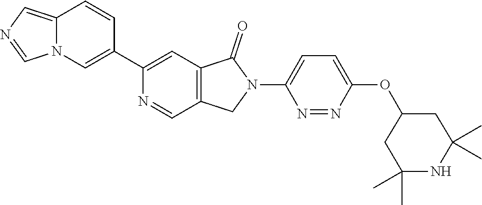 [SMILES: CC1(C)CC(Oc2ccc(N3Cc4cnc(-c5ccc6cncn6c5)cc4C3=O)nn2)CC(C)(C)N1] | 6-(imidazo[1,5-a]pyridin-6-yl)-2- (6-((2,2,6,6-tetramethylpiperidin- 4-yl)oxy)pyridazin-3-yl)-2,3- dihydro-1H-pyrrolo[3,4-c]pyridin- 1-one |
| 30 | 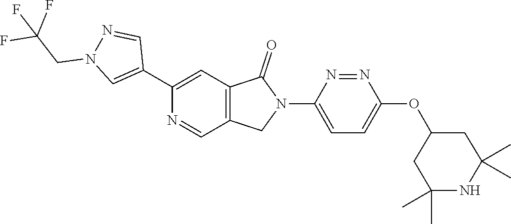 [SMILES: CC1(C)CC(Oc2ccc(N3Cc4cnc(-c5cnn(CC(F)(F)F)c5)cc4C3=O)nn2)CC(C)(C)N1] | 2-(6-((2,2,6,6- tetramethylpiperidin-4- yl)oxy)pyridazin-3-yl)-6-(1-(2,2,2- trifluoroethyl)-1H-pyrazol-4-yl)- 2,3-dihydro-1H-pyrrolo[3,4- c]pyridin-1-one |
| 31 | 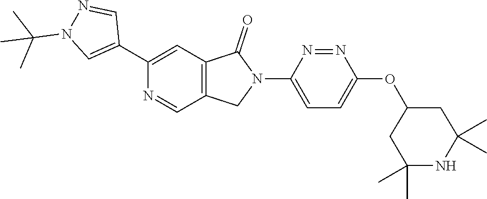 [SMILES: CC1(C)CC(Oc2ccc(N3Cc4cnc(-c5cnn(C(C)(C)C)c5)cc4C3=O)nn2)CC(C)(C)N1] | 6-(1-(tert-butyl)-1H-pyrazol-4-yl)- 2-(6-((2,2,6,6- tetramethylpiperidin-4- yl)oxy)pyridazin-3-yl)-2,3- dihydro-1H-pyrrolo[3,4-c]pyridin- 1-one |
| 35 | 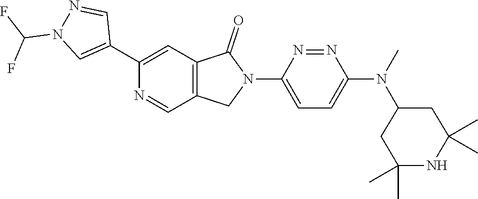 [SMILES: CN(c1ccc(N2Cc3cnc(-c4cnn(C(F)F)c4)cc3C2=O)nn1)C1CC(C)(C)NC(C)(C)C1] | 6-(1-(difluoromethyl)-1H-pyrazol- 4-yl)-2-(6-(methyl(2,2,6,6- tetramethylpiperidin-4- yl)amino)pyridazin-3-yl)-2,3- dihydro-1H-pyrrolo[3,4-c]pyridin- 1-one |
| 43 | 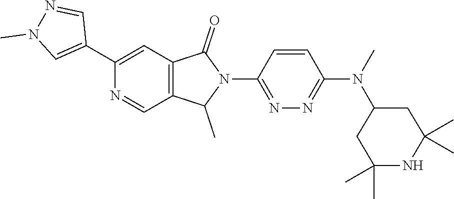 | 3-methyl-2-(6-(methyl(2,2,6,6- tetramethylpiperidin-4- yl)amino)pyridazin-3-yl)-6-(1- methyl-1H-pyrazol-4-yl)-2,3- dihydro-1H-pyrrolo[3,4-c]pyridin- 1-one |
| 47 | 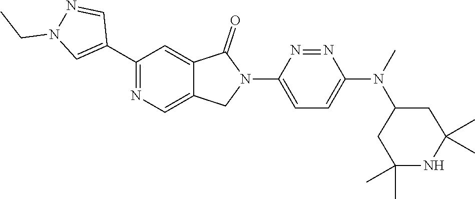 [SMILES: CCn1cc(-c2cc3c(cn2)CN(c2ccc(N(C)C4CC(C)(C)NC(C)(C)C4)nn2)C3=O)cn1] | 6-(1-ethyl-1H-pyrazol-4-yl)-2-(6- (methyl(2,2,6,6- tetramethylpiperidin-4- yl)amino)pyridazin-3-yl)-2,3- dihydro-1H-pyrrolo[3,4-c]pyridin- 1-one |
| 49 | 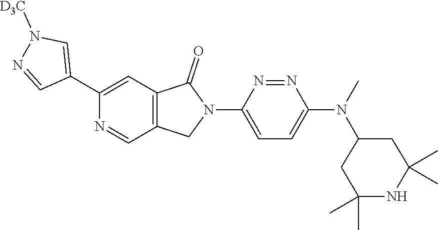 [SMILES: [2H]C([2H])([2H])n1cc(-c2cc3c(cn2)CN(c2ccc(N(C)C4CC(C)(C)NC(C)(C)C4)nn2)C3=O)cn1] | 2-(6-(methyl(2,2,6,6- tetramethylpiperidin-4- yl)amino)pyridazin-3-yl)-6-(1- (methyl-d3)-1H-pyrazol-4-yl)-2,3- dihydro-1H-pyrrolo[3,4-c]pyridin- 1-one |
| 51 | 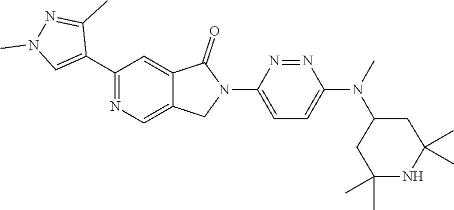 | 6-(1,3-dimethyl-1H-pyrazol-4-yl)- 2-(6-(methyl(2,2,6,6- tetramethylpiperidin-4- yl)amino)pyridazin-3-yl)-2,3- dihydro-1H-pyrrolo[3,4-c]pyridin- 1-one |
| 54 | 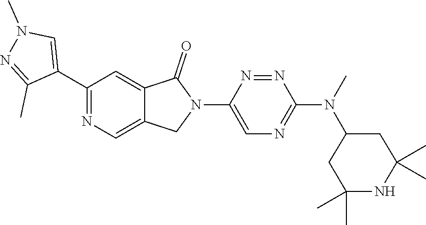 [SMILES: Cc1nn(C)cc1-c1cc2c(cn1)CN(c1cnc(N(C)C3CC(C)(C)NC(C)(C)C3)nn1)C2=O] | 6-(1,3-dimethyl-1H-pyrazol-4-yl)- 2-(3-(methyl(2,2,6,6- tetramethylpiperidin-4-yl)amino)- 1,2,4-triazin-6-yl)-2,3-dihydro-1H- pyrrolo[3,4-c]pyridin-1-one |
| 56 | 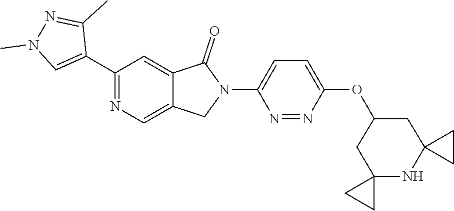 [SMILES: Cc1nn(C)cc1-c1cc2c(cn1)CN(c1ccc(OC3CC4(CC4)NC4(CC4)C3)nn1)C2=O] | 2-(6-((4- azadispiro[2.1.25.33]decan-9- yl)oxy)pyridazin-3-yl)-6-(1,3- dimethyl-1H-pyrazol-4-yl)-2,3- dihydro-1H-pyrrolo[3,4-c]pyridin- 1-one |
| 57 | 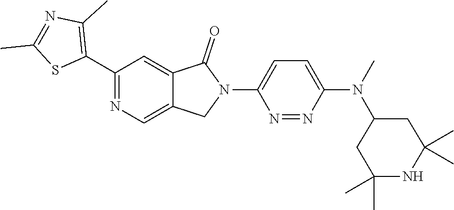 | 6-(2,4-dimethylthiazol-5-yl)-2-(6- (methyl(2,2,6,6- tetramethylpiperidin-4- yl)amino)pyridazin-3-yl)-2,3- dihydro-1H-pyrrolo[3,4-c]pyridin- 1-one |
| 58 | 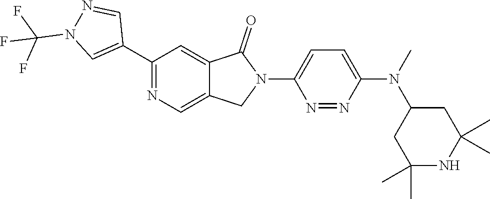 [SMILES: CN(c1ccc(N2Cc3cnc(-c4cnn(C(F)(F)F)c4)cc3C2=O)nn1)C1CC(C)(C)NC(C)(C)C1] | 2-(6-(methyl(2,2,6,6- tetramethylpiperidin-4- yl)amino)pyridazin-3-yl)-6-(1- (trifluoromethyl)-1H-pyrazol-4- yl)-2,3-dihydro-1H-pyrrolo[3,4- c]pyridin-1-one |
| 59 | 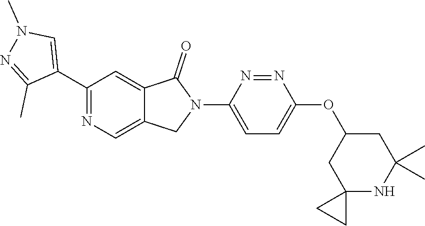 [SMILES: Cc1nn(C)cc1-c1cc2c(cn1)CN(c1ccc(OC3CC(C)(C)NC4(CC4)C3)nn1)C2=O] | 6-(1,3-dimethyl-1H-pyrazol-4-yl)- 2-(6-((5,5-dimethyl-4- azaspiro[2.5]octan-7- yl)oxy)pyridazin-3-yl)-2,3- dihydro-1H-pyrrolo[3,4-c]pyridin- 1-one |
| 63 | 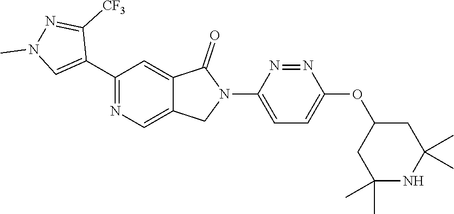 | 6-(1-methyl-3-(trifluoromethyl)- 1H-pyrazol-4-yl)-2-(6-((2,2,6,6- tetramethylpiperidin-4- yl)oxy)pyridazin-3-yl)-2,3- dihydro-1H-pyrrolo[3,4-c]pyridin- 1-one |
| 64 | 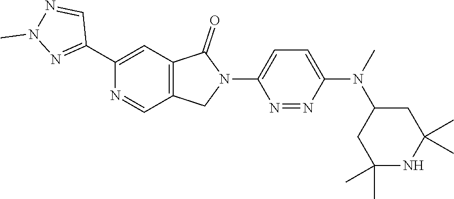 | 2-(6-(methyl(2,2,6,6- tetramethylpiperidin-4- yl)amino)pyridazin-3-yl)-6-(2- methyl-2H-1,2,3-triazol-4-yl)-2,3- dihydro-1H-pyrrolo[3,4-c]pyridin- 1-one |
| 65 | 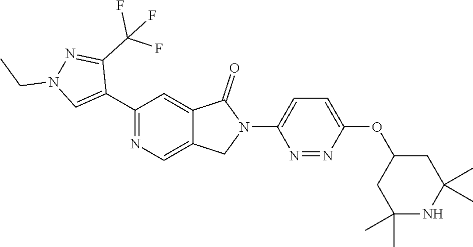 [SMILES: CCn1cc(-c2cc3c(cn2)CN(c2ccc(OC4CC(C)(C)NC(C)(C)C4)nn2)C3=O)c(C(F)(F)F)n1] | 6-(1-ethyl-3-(trifluoromethyl)-1H- pyrazol-4-yl)-2-(6-((2,2,6,6- tetramethylpiperidin-4- yl)oxy)pyridazin-3-yl)-2,3- dihydro-1H-pyrrolo[3,4-c]pyridin- 1-one |
| 66 | 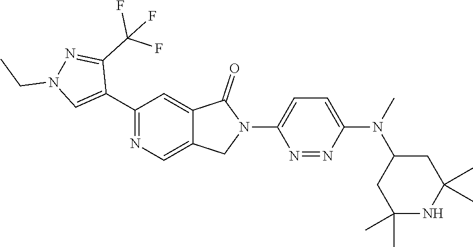 [SMILES: CCn1cc(-c2cc3c(cn2)CN(c2ccc(N(C)C4CC(C)(C)NC(C)(C)C4)nn2)C3=O)c(C(F)(F)F)n1] | 6-(1-ethyl-3-(trifluoromethyl)-1H- pyrazol-4-yl)-2-(6-(methyl(2,2,6,6- tetramethylpiperidin-4- yl)amino)pyridazin-3-yl)-2,3- dihydro-1H-pyrrolo[3,4-c]pyridin- 1-one |
| 71 | 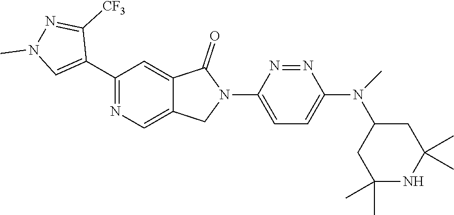 | 2-(6-(methyl(2,2,6,6- tetramethylpiperidin-4- yl)amino)pyridazin-3-yl)-6-(1- methyl-3-(trifluoromethyl)-1H- pyrazol-4-yl)-2,3-dihydro-1H- pyrrolo[3,4-c]pyridin-1-one |
| 75 | 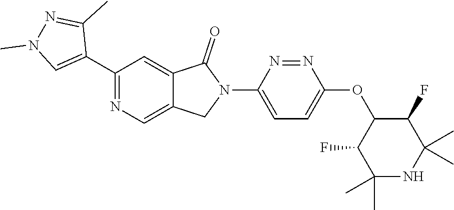 | 2-(6-(((3S,5S)-3,5-difluoro- 2,2,6,6-tetramethylpiperidin-4- yl)oxy)pyridazin-3-yl)-6-(1,3- dimethyl-1H-pyrazol-4-yl)-2,3- dihydro-1H-pyrrolo[3,4-c]pyridin- 1-one |
| 76 | 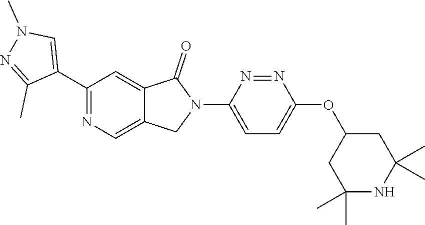 [SMILES: Cc1nn(C)cc1-c1cc2c(cn1)CN(c1ccc(OC3CC(C)(C)NC(C)(C)C3)nn1)C2=O] | 6-(1,3-dimethyl-1H-pyrazol-4-yl)- 2-(6-((2,2,6,6- tetramethylpiperidin-4- yl)oxy)pyridazin-3-yl)-2,3- dihydro-1H-pyrrolo[3,4-c]pyridin- 1-one |
| 78 | 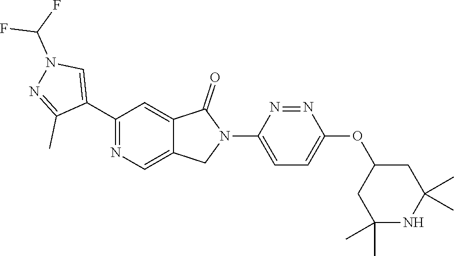 [SMILES: Cc1nn(C(F)F)cc1-c1cc2c(cn1)CN(c1ccc(OC3CC(C)(C)NC(C)(C)C3)nn1)C2=O] | 6-(1-(difluoromethyl)-3-methyl- 1H-pyrazol-4-yl)-2-(6-((2,2,6,6- tetramethylpiperidin-4- yl)oxy)pyridazin-3-yl)-2,3- dihydro-1H-pyrrolo[3,4-c]pyridin- 1-one |
| 80 | 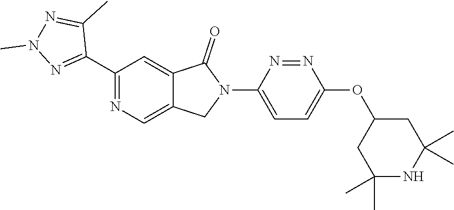 | 6-(2,5-dimethyl-2H-1,2,3-triazol- 4-yl)-2-(6-((2,2,6,6- tetramethylpiperidin-4- yl)oxy)pyridazin-3-yl)-2,3- dihydro-1H-pyrrolo[3,4-c]pyridin- 1-one |
| 81 | 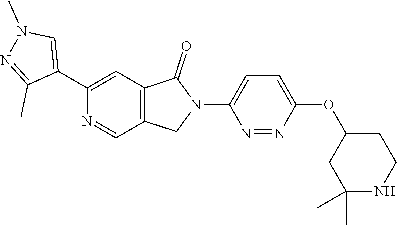 [SMILES: Cc1nn(C)cc1-c1cc2c(cn1)CN(c1ccc(OC3CCNC(C)(C)C3)nn1)C2=O] | 6-(1,3-dimethyl-1H-pyrazol-4-yl)- 2-(6-((2,2-dimethylpiperidin-4- yl)oxy)pyridazin-3-yl)-2,3- dihydro-1H-pyrrolo[3,4-c]pyridin- 1-one |
| 83 | 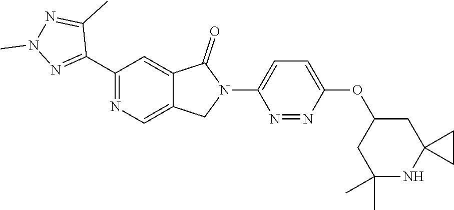 [SMILES: Cc1nn(C)nc1-c1cc2c(cn1)CN(c1ccc(OC3CC(C)(C)NC4(CC4)C3)nn1)C2=O] | 6-(2,5-dimethyl-2H-1,2,3-triazol- 4-yl)-2-(6-((5,5-dimethyl-4- azaspiro[2.5]octan-7- yl)oxy)pyridazin-3-yl)-2,3- dihydro-1H-pyrrolo[3,4-c]pyridin- 1-one |
| 84 | 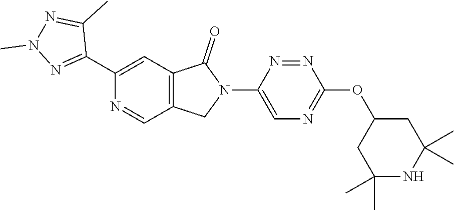 | 6-(1,3-dimethyl-1H-pyrazol-4-yl)- 2-(3-((2,2,6,6- tetramethylpiperidin-4-yl)oxy)- 1,2,4-triazin-6-yl)-2,3-dihydro-1H- pyrrolo[3,4-c]pyridin-1-one |
| 86 | 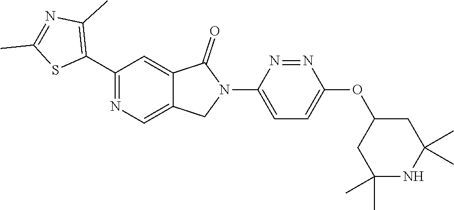 | 6-(2,4-dimethylthiazol-5-yl)-2-(6- ((2,2,6,6-tetramethylpiperidin-4- yl)oxy)pyridazin-3-yl)-2,3- dihydro-1H-pyrrolo[3,4-c]pyridin- 1-one |
| 88 | 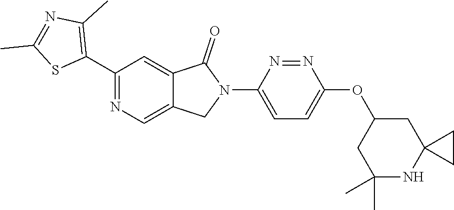 [SMILES: Cc1nc(C)c(-c2cc3c(cn2)CN(c2ccc(OC4CC(C)(C)NC5(CC5)C4)nn2)C3=O)s1] | 2-(6-((5,5-dimethyl-4- azaspiro[2.5]octan-7- yl)oxy)pyridazin-3-yl)-6-(2,4- dimethylthiazol-5-yl)-2,3-dihydro- 1H-pyrrolo[3,4-c]pyridin-1-one |
| 91 | 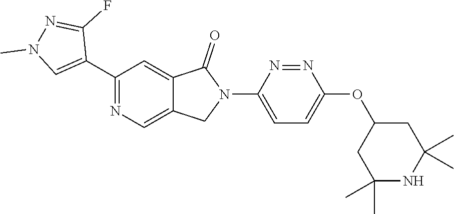 | 6-(3-fluoro-1-methyl-1H-pyrazol- 4-yl)-2-(6-((2,2,6,6- tetramethylpiperidin-4- yl)oxy)pyridazin-3-yl)-2,3- dihydro-1H-pyrrolo[3,4-c]pyridin- 1-one |
| 92 | 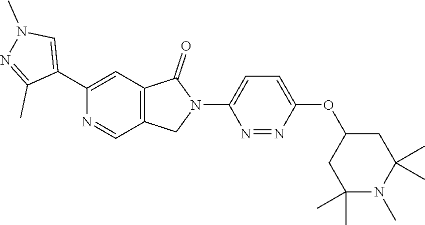 [SMILES: Cc1nn(C)cc1-c1cc2c(cn1)CN(c1ccc(OC3CC(C)(C)N(C)C(C)(C)C3)nn1)C2=O] | 6-(1,3-dimethyl-1H-pyrazol-4-yl)- 2-(6-((1,2,2,6,6- pentamethylpiperidin-4- yl)oxy)pyridazin-3-yl)-2,3- dihydro-1H-pyrrolo[3,4-c]pyridin- 1-one |
| 101 | 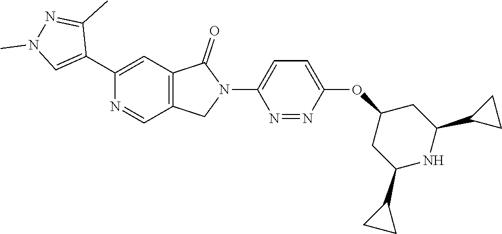 [SMILES: Cc1nn(C)cc1-c1cc2c(cn1)CN(c1ccc(O[C@H]3C[C@@H](C4CC4)N[C@@H](C4CC4)C3)nn1)C2=O] | 2-(6-(((2R,4s,6S)-2,6- dicyclopropylpiperidin-4- yl)oxy)pyridazin-3-yl)-6-(1,3- dimethyl-1H-pyrazol-4-yl)-2,3- dihydro-1H-pyrrolo[3,4-c]pyridin- 1-one |
| 103 | 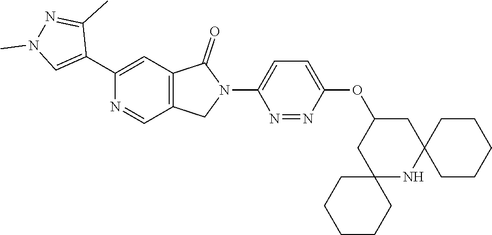 [SMILES: Cc1nn(C)cc1-c1cc2c(cn1)CN(c1ccc(OC3CC4(CCCCC4)NC4(CCCCC4)C3)nn1)C2=O] | 2-(6-((7- azadispiro[5.1.5⁸.3⁶]hexadecan-15- yl)oxy)pyridazin-3-yl)-6-(1,3- dimethyl-1H-pyrazol-4-yl)-2,3- dihydro-1H-pyrrolo[3,4-c]pyridin- 1-one |
| 104 | 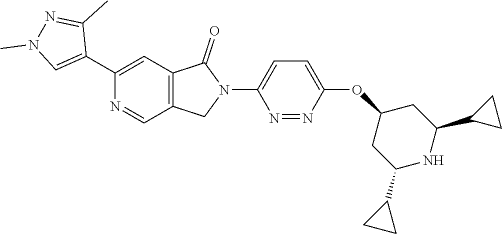 | 2-(6-(((2S,6S)-2,6- dicyclopropylpiperidin-4- yl)oxy)pyridazin-3-yl)-6-(1,3- dimethyl-1H-pyrazol-4-yl)-2,3- dihydro-1H-pyrrolo[3,4-c]pyridin- 1-one |
| 109 | 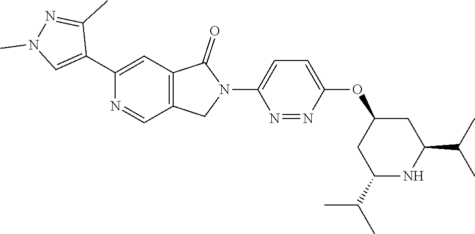 | 2-(6-(((2S,6S)-2,6- diisopropylpiperidin-4- yl)oxy)pyridazin-3-yl)-6-(1,3- dimethyl-1H-pyrazol-4-yl)-2,3- dihydro-1H-pyrrolo[3,4-c]pyridin- 1-one |
| 113 | 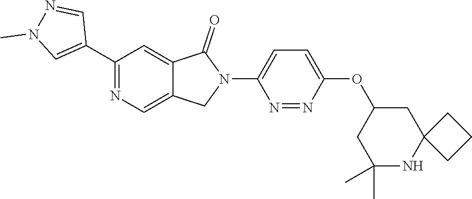 [SMILES: Cn1cc(-c2cc3c(cn2)CN(c2ccc(OC4CC(C)(C)NC5(CCC5)C4)nn2)C3=O)cn1] | 2-(6-((6,6-dimethyl-5- azaspiro[3.5]nonan-8- yl)oxy)pyridazin-3-yl)-6-(1- methyl-1H-pyrazol-4-yl)-2,3- dihydro-1H-pyrrolo[3,4-c]pyridin- 1-one |
| 115 | 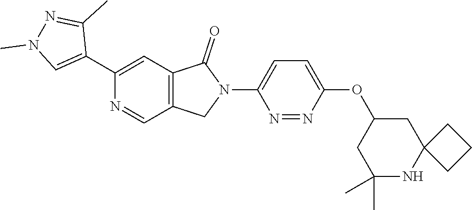 [SMILES: Cc1nn(C)cc1-c1cc2c(cn1)CN(c1ccc(OC3CC(C)(C)NC4(CCC4)C3)nn1)C2=O] | 6-(1,3-dimethyl-1H-pyrazol-4-yl)- 2-(6-((6,6-dimethyl-5- azaspiro[3.5]nonan-8- yl)oxy)pyridazin-3-yl)-2,3- dihydro-1H-pyrrolo[3,4-c]pyridin- 1-one |
| 119 | 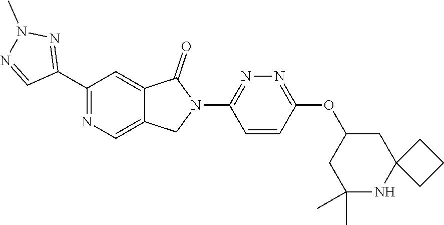 [SMILES: Cn1ncc(-c2cc3c(cn2)CN(c2ccc(OC4CC(C)(C)NC5(CCC5)C4)nn2)C3=O)n1] | 2-(6-((6,6-dimethyl-5- azaspiro[3.5]nonan-8- yl)oxy)pyridazin-3-yl)-6-(2- methyl-2H-1,2,3-triazol-4-yl)-2,3- dihydro-1H-pyrrolo[3,4-c]pyridin- 1-one |
| 134 | 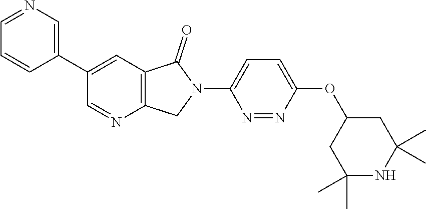 [SMILES: CC1(C)CC(Oc2ccc(N3Cc4ncc(-c5cccnc5)cc4C3=O)nn2)CC(C)(C)N1] | 3-(pyridin-3-yl)-6-(6-((2,2,6,6- tetramethylpiperidin-4- yl)oxy)pyridazin-3-yl)-6,7- dihydro-5H-pyrrolo[3,4-b]pyridin- 5-one |
| 135 | 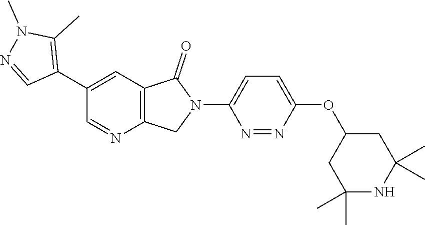 [SMILES: Cc1c(-c2cnc3c(c2)C(=O)N(c2ccc(OC4CC(C)(C)NC(C)(C)C4)nn2)C3)cnn1C] | 3-(1,5-dimethyl-1H-pyrazol-4-yl)- 6-(6-((2,2,6,6- tetramethylpiperidin-4- yl)oxy)pyridazin-3-yl)-6,7- dihydro-5H-pyrrolo[3,4-b]pyridin- 5-one |
| 138 | 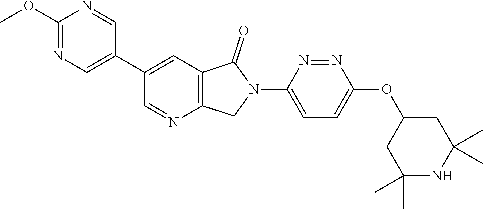 | 3-(2-methoxypyrimidin-5-yl)-6-(6- ((2,2,6,6-tetramethylpiperidin-4- yl)oxy)pyridazin-3-yl)-6,7- dihydro-5H-pyrrolo[3,4-b]pyridin- 5-one |
| 139 | 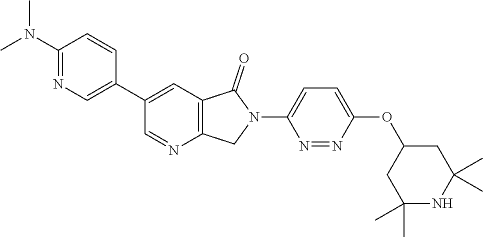 | 3-(6-(dimethylamino)pyridin-3- yl)-6-(6-((2,2,6,6- tetramethylpiperidin-4- yl)oxy)pyridazin-3-yl)-6,7- dihydro-5H-pyrrolo[3,4-b]pyridin- 5-one |
| 140 | 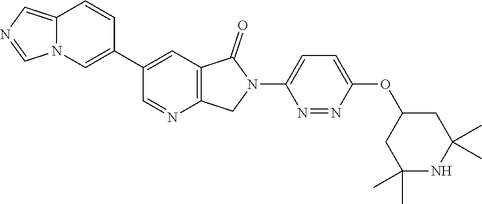 [SMILES: CC1(C)CC(Oc2ccc(N3Cc4ncc(-c5ccc6cncn6c5)cc4C3=O)nn2)CC(C)(C)N1] | 3-(imidazo[1,5-a]pyridin-6-yl)-6- (6-((2,2,6,6-tetramethylpiperidin- 4-yl)oxy)pyridazin-3-yl)-6,7- dihydro-5H-pyrrolo[3,4-b]pyridin- 5-one |
| 141 | 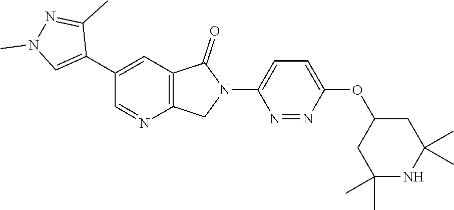 | 3-(1,3-dimethyl-1H-pyrazol-4-yl)- 6-(6-((2,2,6,6- tetramethylpiperidin-4- yl)oxy)pyridazin-3-yl)-6,7- dihydro-5H-pyrrolo[3,4-b]pyridin- 5-one |
| 142 | 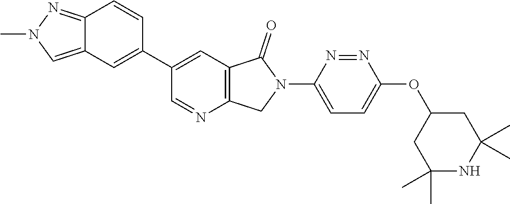 | 3-(2-methyl-2H-indazol-5-yl)-6- (6-((2,2,6,6-tetramethylpiperidin- 4-yl)oxy)pyridazin-3-yl)-6,7- dihydro-5H-pyrrolo[3,4-b]pyridin- 5-one |
| 143 | 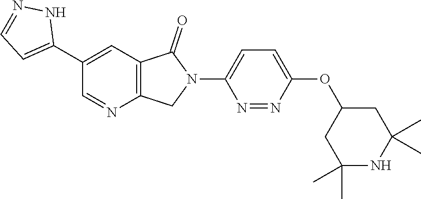 [SMILES: CC1(C)CC(Oc2ccc(N3Cc4ncc(-c5ccn[nH]5)cc4C3=O)nn2)CC(C)(C)N1] | 3-(1H-pyrazol-5-yl)-6-(6-((2,2,6,6- tetramethylpiperidin-4- yl)oxy)pyridazin-3-yl)-6,7- dihydro-5H-pyrrolo[3,4-b]pyridin- 5-one |
| 144 | 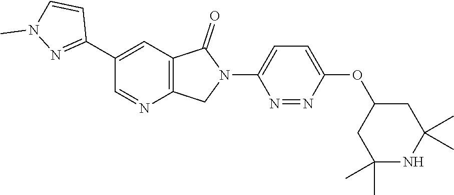 | 3-(1-methyl-1H-pyrazol-3-yl)-6- (6-((2,2,6,6-tetramethylpiperidin- 4-yl)oxy)pyridazin-3-yl)-6,7- dihydro-5H-pyrrolo[3,4-b]pyridin- 5-one |
| 148 | 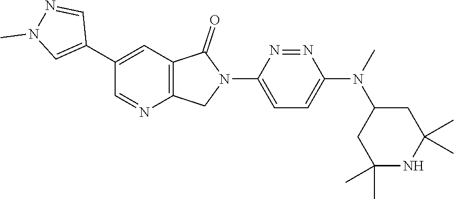 | 6-(6-(methyl(2,2,6,6- tetramethylpiperidin-4- yl)amino)pyridazin-3-yl)-3-(1- methyl-1H-pyrazol-4-yl)-6,7- dihydro-5H-pyrrolo[3,4-b]pyridin- 5-one |
| 149 | 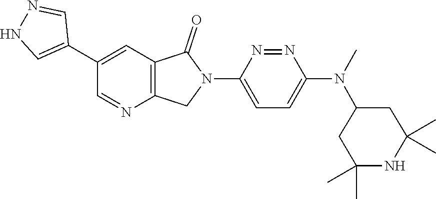 [SMILES: CN(c1ccc(N2Cc3ncc(-c4cn[nH]c4)cc3C2=O)nn1)C1CC(C)(C)NC(C)(C)C1] | 6-(6-(methyl(2,2,6,6- tetramethylpiperidin-4- yl)amino)pyridazin-3-yl)-3-(1H- pyrazol-4-yl)-6,7-dihydro-5H- pyrrolo[3,4-b]pyridin-5-one |
| 150 | 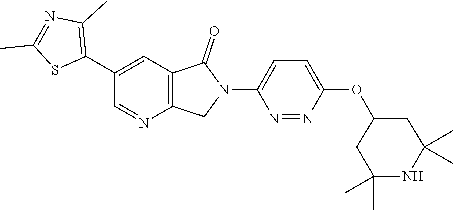 | 3-(2,4-dimethylthiazol-5-yl)-6-(6- ((2,2,6,6-tetramethylpiperidin-4- yl)oxy)pyridazin-3-yl)-6,7- dihydro-5H-pyrrolo[3,4-b]pyridin- 5-one |
| 156 | 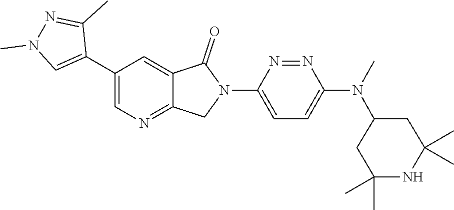 | 3-(1,3-dimethyl-1H-pyrazol-4-yl)- 6-(6-(methyl(2,2,6,6- tetramethylpiperidin-4- yl)amino)pyridazin-3-yl)-6,7- dihydro-5H-pyrrolo[3,4-b]pyridin- 5-one |
| 160 | 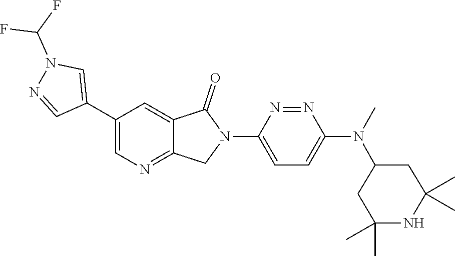 [SMILES: CN(c1ccc(N2Cc3ncc(-c4cnn(C(F)F)c4)cc3C2=O)nn1)C1CC(C)(C)NC(C)(C)C1] | 3-(1 -(difluoromethyl)-1H-pyrazol- 4-yl)-6-(6-(methyl(2,2,6,6- tetramethylpiperidin-4- yl)amino)pyridazin-3-yl)-6,7- dihydro-5H-pyrrolo[3,4-b]pyridin- 5-one |
| 161 | 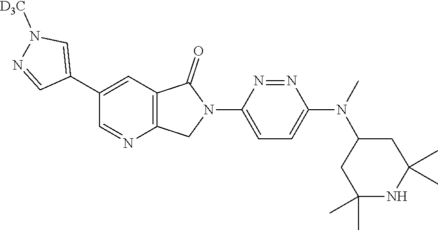 [SMILES: [2H]C([2H])([2H])n1cc(-c2cnc3c(c2)C(=O)N(c2ccc(N(C)C4CC(C)(C)NC(C)(C)C4)nn2)C3)cn1] | 6-(6-(methyl(2,2,6,6- tetramethylpiperidin-4- yl)amino)pyridazin-3-yl)-3-(1- (methyl-d3)-1H-pyrazol-4-yl)-6,7- dihydro-5H-pyrrolo[3,4-b]pyridin- 5-one |
| 162 | 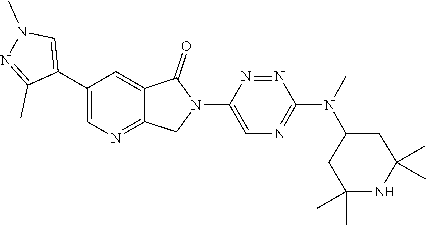 [SMILES: Cc1nn(C)cc1-c1cnc2c(c1)C(=O)N(c1cnc(N(C)C3CC(C)(C)NC(C)(C)C3)nn1)C2] | 3-(1,3-dimethyl-1H-pyrazol-4-yl)- 6-(3-(methyl(2,2,6,6- tetramethylpiperidin-4-yl)amino)- 1,2,4-triazin-6-yl)-6,7-dihydro-5H- pyrrolo[3,4-b]pyridin-5-one |
| 163 | 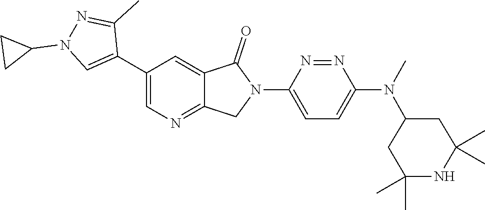 [SMILES: Cc1nn(C2CC2)cc1-c1cnc2c(c1)C(=O)N(c1ccc(N(C)C3CC(C)(C)NC(C)(C)C3)nn1)C2] | 3-(1-cyclopropyl-3-methyl-1H- pyrazol-4-yl)-6-(6-(methyl(2,2,6,6- tetramethylpiperidin-4- yl)amino)pyridazin-3-yl)-6,7- dihydro-5H-pyrrolo[3,4-b]pyridin- 5-one |
| 167 | 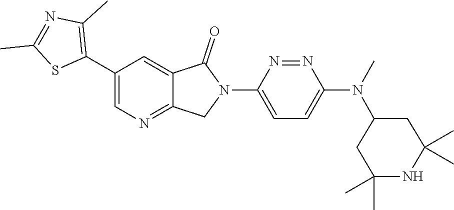 | 3-(2,4-dimethylthiazol-5-yl)-6-(6- (methyl(2,2,6,6- tetramethylpiperidin-4- yl)amino)pyridazin-3-yl)-6,7- dihydro-5H-pyrrolo[3,4-b]pyridin- 5-one |
| 168 | 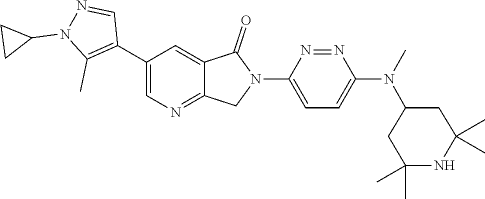 [SMILES: Cc1c(-c2cnc3c(c2)C(=O)N(c2ccc(N(C)C4CC(C)(C)NC(C)(C)C4)nn2)C3)cnn1C1CC1] | 3-(1-cyclopropyl-5-methyl-1H- pyrazol-4-yl)-6-(6-(methyl(2,2,6,6- tetramethylpiperidin-4- yl)amino)pyridazin-3-yl)-6,7- dihydro-5H-pyrrolo[3,4-b]pyridin- 5-one |
| 172 | 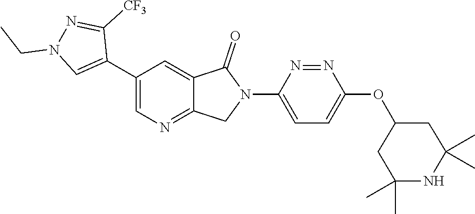 [SMILES: CCn1cc(-c2cnc3c(c2)C(=O)N(c2ccc(OC4CC(C)(C)NC(C)(C)C4)nn2)C3)c(C(F)(F)F)n1] | 3-(1-ethyl-3-(trifluoromethyl)-1H- pyrazol-4-yl)-6-(6-((2,2,6,6- tetramethylpiperidin-4- yl)oxy)pyridazin-3-yl)-6,7- dihydro-5H-pyrrolo[3,4-b]pyridin- 5-one |
| 173 | 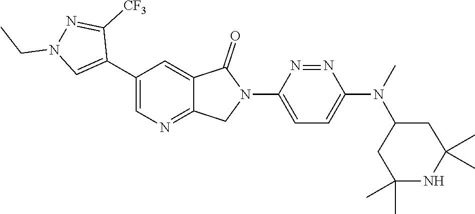 [SMILES: CCn1cc(-c2cnc3c(c2)C(=O)N(c2ccc(N(C)C4CC(C)(C)NC(C)(C)C4)nn2)C3)c(C(F)(F)F)n1] | 3-(1-ethyl-3-(trifluoromethyl)-1H- pyrazol-4-yl)-6-(6-(methyl(2,2,6,6- tetramethylpiperidin-4- yl)amino)pyridazin-3-yl)-6,7- dihydro-5H-pyrrolo[3,4-b]pyridin- 5-one |
| 176 | 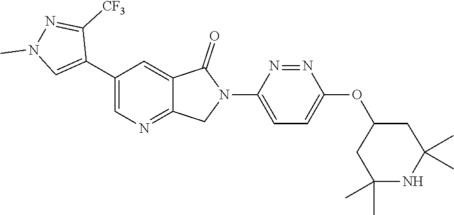 | 3-(1-methyl-3-(trifluoromethyl)- 1H-pyrazol-4-yl)-6-(6-((2,2,6,6- tetramethylpiperidin-4- yl)oxy)pyridazin-3-yl)-6,7- dihydro-5H-pyrrolo[3,4-b]pyridin- 5-one |
| 177 | 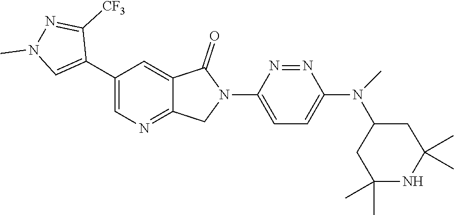 | 6-(6-(methyl(2,2,6,6- tetramethylpiperidin-4- yl)amino)pyridazin-3-yl)-3-(1- methyl-3-(trifluoromethyl)-1H- pyrazol-4-yl)-6,7-dihydro-5H- pyrrolo[3,4-b]pyridin-5-one |
| 179 | 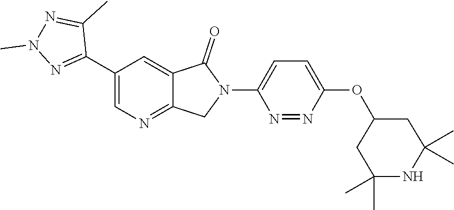 | 3-(2,5-dimethyl-2H-1,2,3-triazol- 4-yl)-6-(6-((2,2,6,6- tetramethylpiperidin-4- yl)oxy)pyridazin-3-yl)-6,7- dihydro-5H-pyrrolo[3,4-b]pyridin- 5-one |
| 180 | 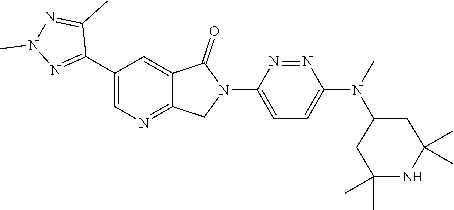 | 3-(2,5-dimethyl-2H-1,2,3-triazol- 4-yl)-6-(6-(methyl(2,2,6,6- tetramethylpiperidin-4- yl)amino)pyridazin-3-yl)-6,7- dihydro-5H-pyrrolo[3,4-b]pyridin- 5-one |
| 182 | 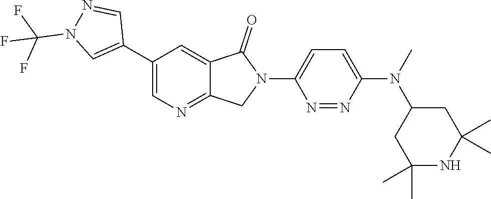 [SMILES: CN(c1ccc(N2Cc3ncc(-c4cnn(C(F)(F)F)c4)cc3C2=O)nn1)C1CC(C)(C)NC(C)(C)C1] | 6-(6-(methyl(2,2,6,6- tetramethylpiperidin-4- yl)amino)pyridazin-3-yl)-3-(1- (trifluoromethyl)-1H-pyrazol-4- yl)-6,7-dihydro-5H-pyrrolo[3,4- b]pyridin-5-one |
| 184 | 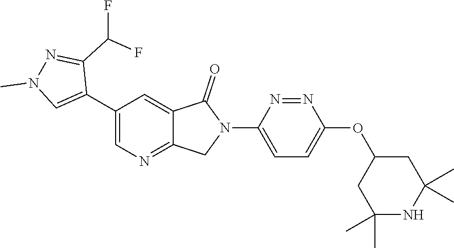 | 3-(3-(difluoromethyl)-1-methyl- 1H-pyrazol-4-yl)-6-(6-((2,2,6,6- tetramethylpiperidin-4- yl)oxy)pyridazin-3-yl)-6,7- dihydro-5H-pyrrolo[3,4-b]pyridin- 5-one |
| 187 | 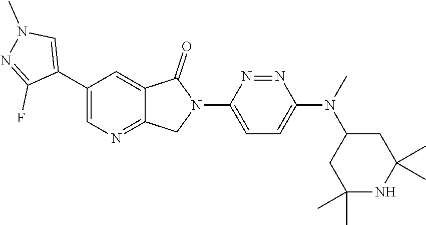 [SMILES: CN(c1ccc(N2Cc3ncc(-c4cn(C)nc4F)cc3C2=O)nn1)C1CC(C)(C)NC(C)(C)C1] | 3-(3-fluoro-1-methyl-1H-pyrazol- 4-yl)-6-(6-(methyl(2,2,6,6- tetramethylpiperidin-4- yl)amino)pyridazin-3-yl)-6,7- dihydro-5H-pyrrolo[3,4-b]pyridin- 5-one |
| 189 | 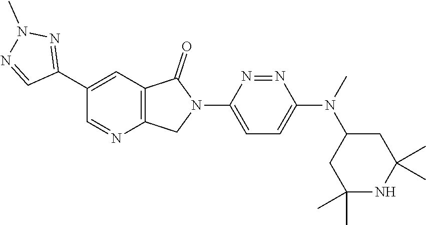 [SMILES: CN(c1ccc(N2Cc3ncc(-c4cnn(C)n4)cc3C2=O)nn1)C1CC(C)(C)NC(C)(C)C1] | 6-(6-(methyl(2,2,6,6- tetramethylpiperidin-4- yl)amino)pyridazin-3-yl)-3-(2- methyl-2H-1,2,3-triazol-4-yl)-6,7- dihydro-5H-pyrrolo[3,4-b]pyridin- 5-one |
| 190 | 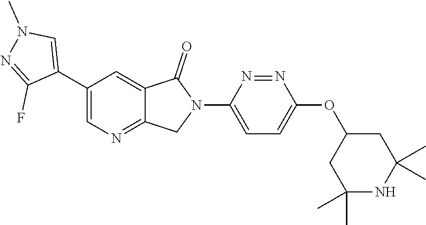 [SMILES: Cn1cc(-c2cnc3c(c2)C(=O)N(c2ccc(OC4CC(C)(C)NC(C)(C)C4)nn2)C3)c(F)n1] | 3-(3-fluoro-1-methyl-1H-pyrazol- 4-yl)-6-(6-((2,2,6,6- tetramethylpiperidin-4- yl)oxy)pyridazin-3-yl)-6,7- dihydro-5H-pyrrolo[3,4-b]pyridin- 5-one |
| 191 | 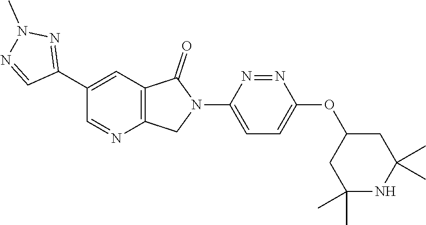 [SMILES: Cn1ncc(-c2cnc3c(c2)C(=O)N(c2ccc(OC4CC(C)(C)NC(C)(C)C4)nn2)C3)n1] | 3-(2-methyl-2H-1,2,3-triazol-4- yl)-6-(6-((2,2,6,6- tetramethylpiperidin-4- yl)oxy)pyridazin-3-yl)-6,7- dihydro-5H-pyrrolo[3,4-b]pyridin- 5-one |
| 193 | 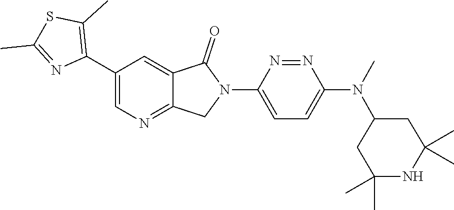 | 3-(2,5-dimethylthiazol-4-yl)-6-(6- (methyl(2,2,6,6- tetramethylpiperidin-4- yl)amino)pyridazin-3-yl)-6,7- dihydro-5H-pyrrolo[3,4-b]pyridin- 5-one |
| 195 | 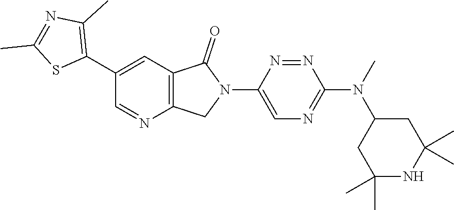 | 3-(2,4-dimethylthiazol-5-yl)-6-(3- (methyl(2,2,6,6- tetramethylpiperidin-4-yl)amino)- 1,2,4-triazin-6-yl)-6,7-dihydro-5H- pyrrolo[3,4-b]pyridin-5-one |
| 198 | 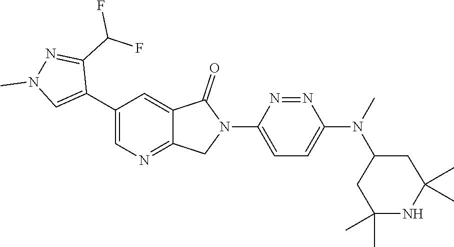 | 3-(3-(difluoromethyl)-1-methyl- 1H-pyrazol-4-yl)-6-(6- (methyl(2,2,6,6- tetramethylpiperidin-4- yl)amino)pyridazin-3-yl)-6,7- dihydro-5H-pyrrolo[3,4-b]pyridin- 5-one |
| 203 | 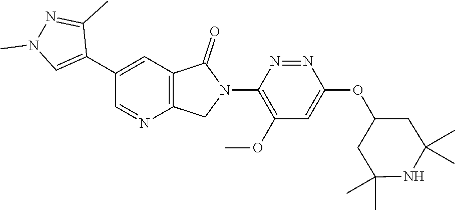 | 3-(1,3-dimethyl-1H-pyrazol-4-yl)- 6-(4-methoxy-6-((2,2,6,6- tetramethylpiperidin-4- yl)oxy)pyridazin-3-yl)-6,7- dihydro-5H-pyrrolo[3,4-b]pyridin- 5-one |
| 205 | 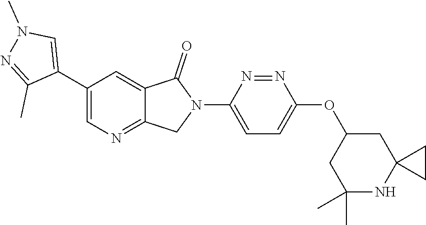 [SMILES: Cc1nn(C)cc1-c1cnc2c(c1)C(=O)N(c1ccc(OC3CC(C)(C)NC4(CC4)C3)nn1)C2] | 3-(1,3-dimethyl-1H-pyrazol-4-yl)- 6-(6-((5,5-dimethyl-4- azaspiro[2.5]octan-7- yl)oxy)pyridazin-3-yl)-6,7- dihydro-5H-pyrrolo[3,4-b]pyridin- 5-one |
| 207 | 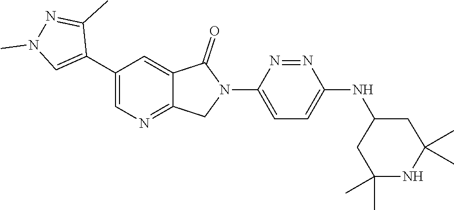 | 3-(1,3-dimethyl-1H-pyrazol-4-yl)- 6-(6-((2,2,6,6- tetramethylpiperidin-4- yl)amino)pyridazin-3-yl)-6,7- dihydro-5H-pyrrolo[3,4-b]pyridin- 5-one |
| 208 | 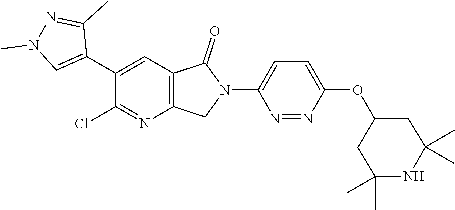 | 2-chloro-3-(1,3-dimethyl-1H- pyrazol-4-yl)-6-(6-((2,2,6,6- tetramethylpiperidin-4- yl)oxy)pyridazin-3-yl)-6,7- dihydro-5H-pyrrolo[3,4-b]pyridin- 5-one |
| 210 | 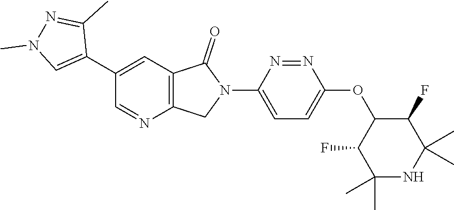 | 6-(6-(((3S,5S)-3,5-difluoro- 2,2,6,6-tetramethylpiperidin-4- yl)oxy)pyridazin-3-yl)-3-(1,3- dimethyl-1H-pyrazol-4-yl)-6,7- dihydro-5H-pyrrolo[3,4-b]pyridin- 5-one |
| 211 | 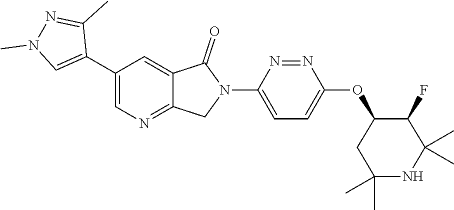 | 3-(1,3-dimethyl-1H-pyrazol-4-yl)- 6-(6-(((3S,4R)-3-fluoro-2,2,6,6- tetramethylpiperidin-4- yl)oxy)pyridazin-3-yl)-6,7- dihydro-5H-pyrrolo[3,4-b]pyridin- 5-one |
| 212 | 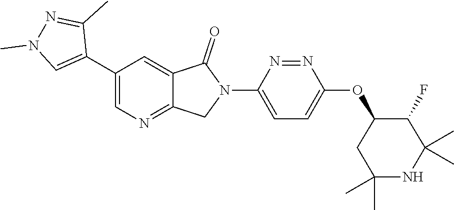 | 3-(1,3-dimethyl-1H-pyrazol-4-yl)- 6-(6-(((3R,4R)-3-fluoro-2,2,6,6- tetramethylpiperidin-4- yl)oxy)pyridazin-3-yl)-6,7- dihydro-5H-pyrrolo[3,4-b]pyridin- 5-one |
| 216 | 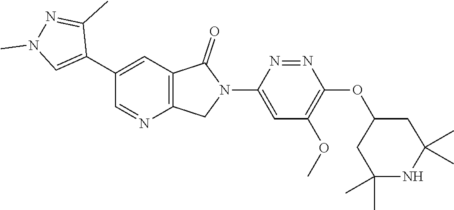 | 3-(1,3-dimethyl-1H-pyrazol-4-yl)- 6-(5-methoxy-6-((2,2,6,6- tetramethylpiperidin-4- yl)oxy)pyridazin-3-yl)-6,7- dihydro-5H-pyrrolo[3,4-b]pyridin- 5-one |
| 218 | 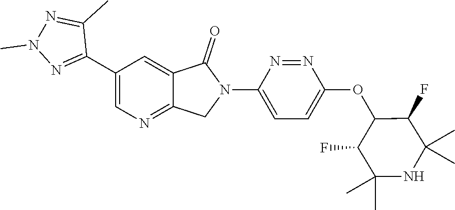 | 6-(6-(((3S,5S)-3,5-difluoro- 2,2,6,6-tetramethylpiperidin-4- yl)oxy)pyridazin-3-yl)-3-(2,5- dimethyl-2H-1,2,3-triazol-4-yl)- 6,7-dihydro-5H-pyrrolo[3,4- b]pyridin-5-one |
| 221 | 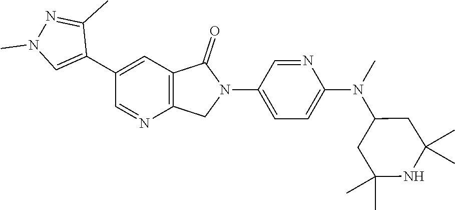 | 3-(1,3-dimethyl-1H-pyrazol-4-yl)- 6-(6-(methyl(2,2,6,6- tetramethylpiperidin-4- yl)amino)pyridin-3-yl)-6,7- dihydro-5H-pyrrolo[3,4-b]pyridin- 5-one |
| 222 | 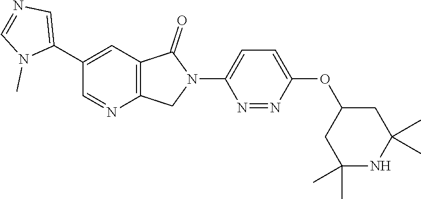 [SMILES: Cn1cncc1-c1cnc2c(c1)C(=O)N(c1ccc(OC3CC(C)(C)NC(C)(C)C3)nn1)C2] | 3-(1-methyl-1H-imidazol-5-yl)-6- (6-((2,2,6,6-tetramethylpiperidin- 4-yl)oxy)pyridazin-3-yl)-6,7- dihydro-5H-pyrrolo[3,4-b]pyridin- 5-one |
| 223 | 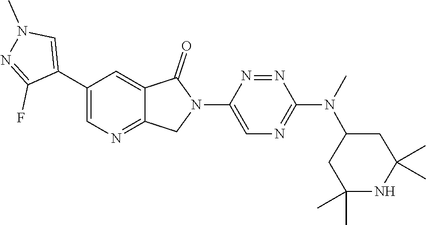 [SMILES: CN(c1ncc(N2Cc3ncc(-c4cn(C)nc4F)cc3C2=O)nn1)C1CC(C)(C)NC(C)(C)C1] | 3-(3-fluoro-1-methyl-1H-pyrazol- 4-yl)-6-(3-(methyl(2,2,6,6- tetramethylpiperidin-4-yl)amino)- 1,2,4-triazin-6-yl)-6,7-dihydro-5H- pyrrolo[3,4-b]pyridin-5-one |
| 224 | 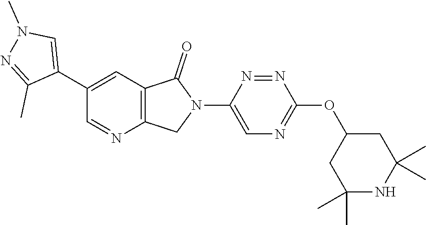 [SMILES: Cc1nn(C)cc1-c1cnc2c(c1)C(=O)N(c1cnc(OC3CC(C)(C)NC(C)(C)C3)nn1)C2] | 3-(1,3-dimethyl-1H-pyrazol-4-yl)- 6-(3-((2,2,6,6- tetramethylpiperidin-4-yl)oxy)- 1,2,4-triazin-6-yl)-6,7-dihydro-5H- pyrrolo[3,4-b]pyridin-5-one |
| 225 | 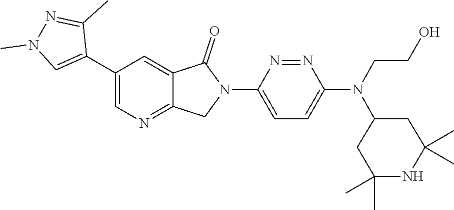 | 3-(1,3-dimethyl-1H-pyrazol-4-yl)- 6-(6-((2-hydroxyethyl)(2,2,6,6- tetramethylpiperidin-4- yl)amino)pyridazin-3-yl)-6,7- dihydro-5H-pyrrolo[3,4-b]pyridin- 5-one |
| 226 | 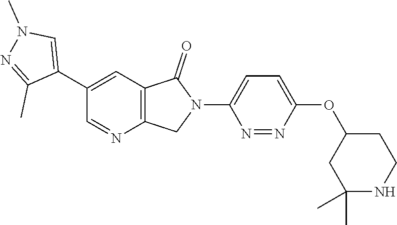 [SMILES: Cc1nn(C)cc1-c1cnc2c(c1)C(=O)N(c1ccc(OC3CCNC(C)(C)C3)nn1)C2] | 3-(1,3-dimethyl-1H-pyrazol-4-yl)- 6-(6-((2,2-dimethylpiperidin-4- yl)oxy)pyridazin-3-yl)-6,7- dihydro-5H-pyrrolo[3,4-b]pyridin- 5-one |
| 228 | 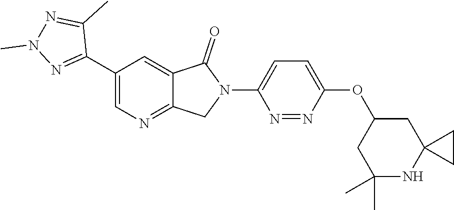 [SMILES: Cc1nn(C)nc1-c1cnc2c(c1)C(=O)N(c1ccc(OC3CC(C)(C)NC4(CC4)C3)nn1)C2] | 3-(2,5-dimethyl-2H-1,2,3-triazol- 4-yl)-6-(6-((5,5-dimethyl-4- azaspiro[2.5]octan-7- yl)oxy)pyridazin-3-yl)-6,7- dihydro-5H-pyrrolo[3,4-b]pyridin- 5-one |
| 230 | 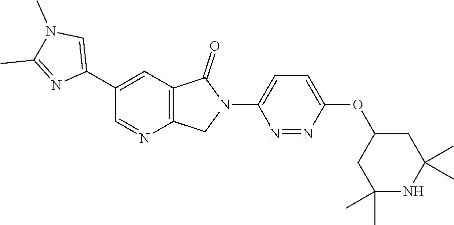 | 3-(1,2-dimethyl-1H-imidazol-4- yl)-6-(6-((2,2,6,6- tetramethylpiperidin-4- yl)oxy)pyridazin-3-yl)-6,7- dihydro-5H-pyrrolo[3,4-b]pyridin- 5-one |
| 231 | 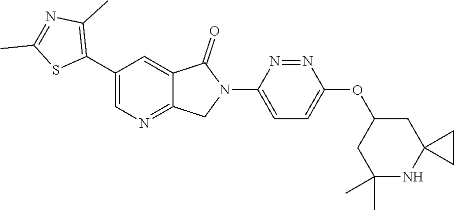 [SMILES: Cc1nc(C)c(-c2cnc3c(c2)C(=O)N(c2ccc(OC4CC(C)(C)NC5(CC5)C4)nn2)C3)s1] | 6-(6-((5,5-dimethyl-4- azaspiro[2.5]octan-7- yl)oxy)pyridazin-3-yl)-3-(2,4- dimethylthiazol-5-yl)-6,7-dihydro- 5H-pyrrolo[3,4-b]pyridin-5-one |
| 232 | 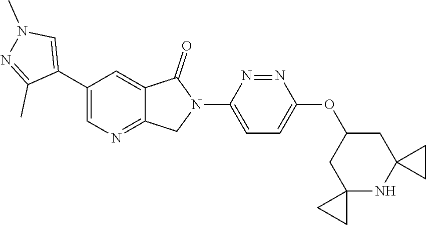 [SMILES: Cc1nn(C)cc1-c1cnc2c(c1)C(=O)N(c1ccc(OC3CC4(CC4)NC4(CC4)C3)nn1)C2] | 6-(6-((4- azadispiro[2.1.25.33]decan-9- yl)oxy)pyridazin-3-yl)-3-(1,3- dimethyl-1H-pyrazol-4-yl)-6,7- dihydro-5H-pyrrolo[3,4-b]pyridin- 5-one |
| 234 | 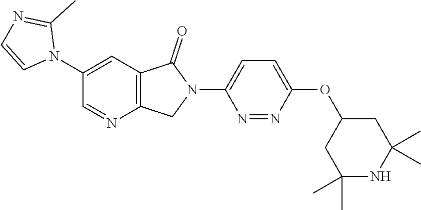 [SMILES: Cc1nccn1-c1cnc2c(c1)C(=O)N(c1ccc(OC3CC(C)(C)NC(C)(C)C3)nn1)C2] | 3-(2-methyl-1H-imidazol-1-yl)-6- (6-((2,2,6,6-tetramethylpiperidin- 4-yl)oxy)pyridazin-3-yl)-6,7- dihydro-5H-pyrrolo[3,4-b]pyridin- 5-one |
| 235 | 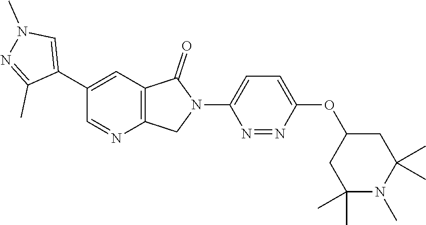 [SMILES: Cc1nn(C)cc1-c1cnc2c(c1)C(=O)N(c1ccc(OC3CC(C)(C)N(C)C(C)(C)C3)nn1)C2] | 3-(1,3-dimethyl-1H-pyrazol-4-yl)- 6-(6-((1,2,2,6,6- pentamethylpiperidin-4- yl)oxy)pyridazin-3-yl)-6,7- dihydro-5H-pyrrolo[3,4-b]pyridin- 5-one |
| 237 | 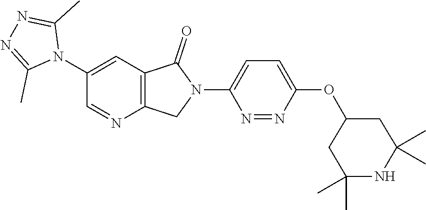 [SMILES: Cc1nnc(C)n1-c1cnc2c(c1)C(=O)N(c1ccc(OC3CC(C)(C)NC(C)(C)C3)nn1)C2] | 3-(3,5-dimethyl-4H-1,2,4-triazol- 4-yl)-6-(6-((2,2,6,6- tetramethylpiperidin-4- yl)oxy)pyridazin-3-yl)-6,7- dihydro-5H-pyrrolo[3,4-b]pyridin- 5-one |
| 241 | 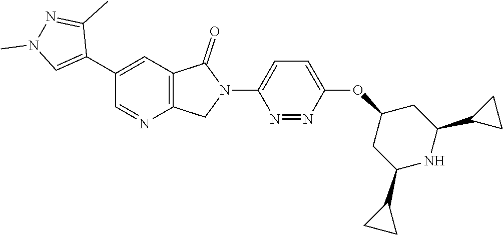 [SMILES: Cc1nn(C)cc1-c1cnc2c(c1)C(=O)N(c1ccc(O[C@H]3C[C@@H](C4CC4)N[C@@H](C4CC4)C3)nn1)C2] | 6-(6-(((2R,4s,6S)-2,6- dicyclopropylpiperidin-4- yl)oxy)pyridazin-3-yl)-3-(1,3- dimethyl-1H-pyrazol-4-yl)-6,7- dihydro-5H-pyrrolo[3,4-b]pyridin- 5-one |
| 243 | 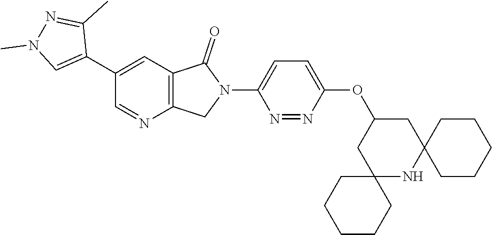 [SMILES: Cc1nn(C)cc1-c1cnc2c(c1)C(=O)N(c1ccc(OC3CC4(CCCCC4)NC4(CCCCC4)C3)nn1)C2] | 6-(6-((7- azadispiro[5.1.58.36]hexadecan-15- yl)oxy)pyridazin-3-yl)-3-(1,3- dimethyl-1H-pyrazol-4-yl)-6,7- dihydro-5H-pyrrolo[3,4-b]pyridin- 5-one |
| 245 | 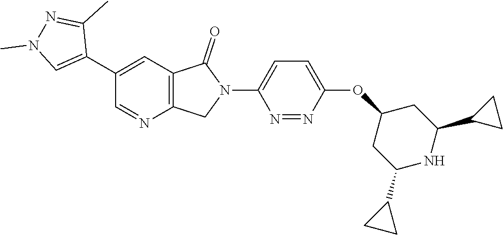 | 6-(6-(((2S,6S)-2,6- dicyclopropylpiperidin-4- yl)oxy)pyridazin-3-yl)-3-(1,3- dimethyl-1H-pyrazol-4-yl)-6,7- dihydro-5H-pyrrolo[3,4-b]pyridin- 5-one |
| 247 | 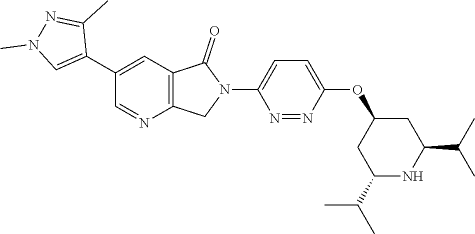 | 6-(6-(((2S,6S)-2,6- diisopropylpiperidin-4- yl)oxy)pyridazin-3-yl)-3-(1,3- dimethyl-1H-pyrazol-4-yl)-6,7- dihydro-5H-pyrrolo[3,4-b]pyridin- 5-one |
| 249 | 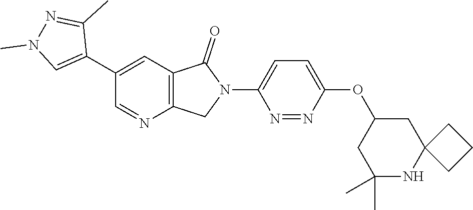 [SMILES: Cc1nn(C)cc1-c1cnc2c(c1)C(=O)N(c1ccc(OC3CC(C)(C)NC4(CCC4)C3)nn1)C2] | 3-(1,3-dimethyl-1H-pyrazol-4-yl)- 6-(6-((6,6-dimethyl-5- azaspiro[3.5]nonan-8- yl)oxy)pyridazin-3-yl)-6,7- dihydro-5H-pyrrolo[3,4-b]pyridin- 5-one |
| 253 | 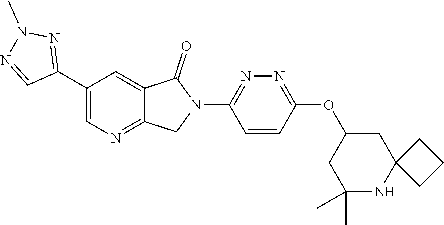 [SMILES: Cn1ncc(-c2cnc3c(c2)C(=O)N(c2ccc(OC4CC(C)(C)NC5(CCC5)C4)nn2)C3)n1] | 6-(6-((6,6-dimethyl-5- azaspiro[3.5]nonan-8- yl)oxy)pyridazin-3-yl)-3-(2- methyl-2H-1,2,3-triazol-4-yl)-6,7- dihydro-5H-pyrrolo[3,4-b]pyridin- 5-one |
| 275 | 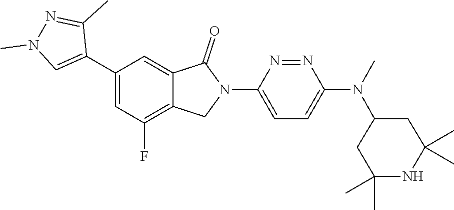 | 6-(1,3-dimethyl-1H-pyrazol-4-yl)- 4-fluoro-2-(6-(methyl(2,2,6,6- tetramethylpiperidin-4- yl)amino)pyridazin-3- yl)isoindolin-1-one |
| 276 | 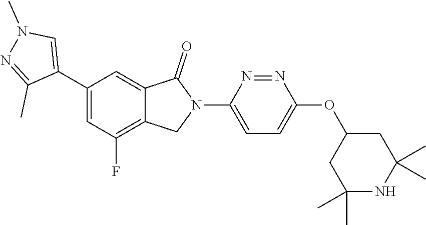 [SMILES: Cc1nn(C)cc1-c1cc(F)c2c(c1)C(=O)N(c1ccc(OC3CC(C)(C)NC(C)(C)C3)nn1)C2] | 6-(1,3-dimethyl-1H-pyrazol-4-yl)- 4-fluoro-2-(6-((2,2,6,6- tetramethylpiperidin-4- yl)oxy)pyridazin-3-yl)isoindolin- 1-one |
| 277 | 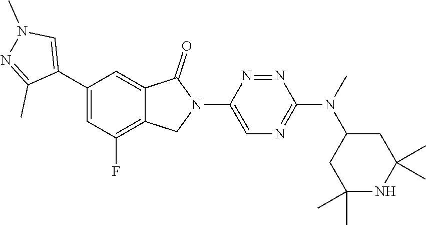 [SMILES: Cc1nn(C)cc1-c1cc(F)c2c(c1)C(=O)N(c1cnc(N(C)C3CC(C)(C)NC(C)(C)C3)nn1)C2] | 6-(1,3-dimethyl-1H-pyrazol-4-yl)- 4-fluoro-2-(3-(methyl(2,2,6,6- tetramethylpiperidin-4-yl)amino)- 1,2,4-triazin-6-yl)isoindolin-1-one |
| 279 | 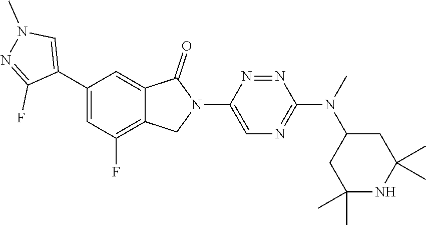 [SMILES: CN(c1ncc(N2Cc3c(F)cc(-c4cn(C)nc4F)cc3C2=O)nn1)C1CC(C)(C)NC(C)(C)C1] | 4-fluoro-6-(3-fluoro-1-methyl-1H- pyrazol-4-yl)-2-(3-(methyl(2,2,6,6- tetramethylpiperidin-4-yl)amino)- 1,2,4-triazin-6-yl)isoindolin-1-one |
| 280 | 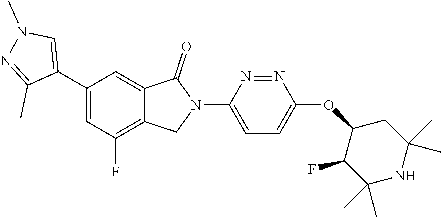 [SMILES: Cc1nn(C)cc1-c1cc(F)c2c(c1)C(=O)N(c1ccc(O[C@H]3CC(C)(C)NC(C)(C)[C@H]3F)nn1)C2] | 6-(1,3-dimethyl-1H-pyrazol-4-yl)- 4-fluoro-2-(6-(((3R,4S)-3-fluoro- 2,2,6,6-tetramethylpiperidin-4- yl)oxy)pyridazin-3-yl)isoindolin- 1-one |
| 282 | 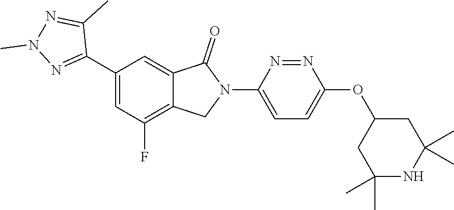 | 6-(2,5-dimethyl-2H-1,2,3-triazol- 4-yl)-4-fluoro-2-(6-((2,2,6,6- tetramethylpiperidin-4- yl)oxy)pyridazin-3-yl)isoindolin- 1-one |
| 284 | 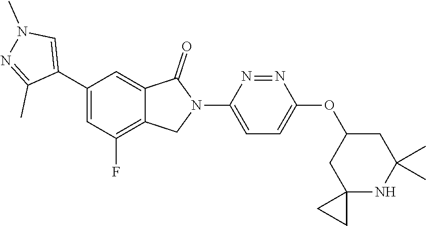 [SMILES: Cc1nn(C)cc1-c1cc(F)c2c(c1)C(=O)N(c1ccc(OC3CC(C)(C)NC4(CC4)C3)nn1)C2] | 6-(1,3-dimethyl-1H-pyrazol-4-yl)- 2-(6-((5,5-dimethyl-4- azaspiro[2.5]octan-7- yl)oxy)pyridazin-3-yl)-4- fluoroisoindolin-1-one |
| 285 | 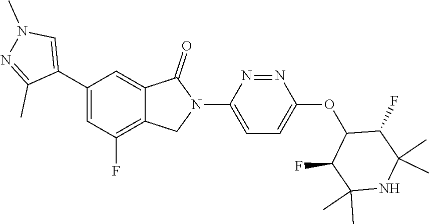 [SMILES: Cc1nn(C)cc1-c1cc(F)c2c(c1)C(=O)N(c1ccc(OC3[C@H](F)C(C)(C)NC(C)(C)[C@H]3F)nn1)C2] | 2-(6-(((3R,5R)-3,5-difluoro- 2,2,6,6-tetramethylpiperidin-4- yl)oxy)pyridazin-3-yl)-6-(1,3- dimethyl-1H-pyrazol-4-yl)-4- fluoroisoindolin-1-one |
| 287 | 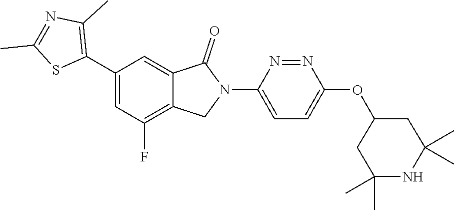 | 6-(2,4-dimethylthiazol-5-yl)-4- fluoro-2-(6-((2,2,6,6- tetramethylpiperidin-4- yl)oxy)pyridazin-3-yl)isoindolin- 1-one |
| 288 | 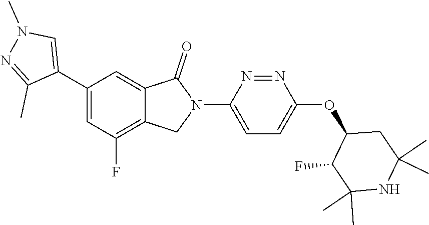 [SMILES: Cc1nn(C)cc1-c1cc(F)c2c(c1)C(=O)N(c1ccc(O[C@H]3CC(C)(C)NC(C)(C)[C@@H]3F)nn1)C2] | 6-(1,3-dimethyl-1H-pyrazol-4-yl)- 4-fluoro-2-(6-(((3S,4S)-3-fluoro- 2,2,6,6-tetramethylpiperidin-4- yl)oxy)pyridazin-3-yl)isoindolin- 1-one |
| 290 | 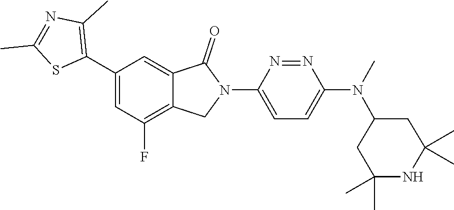 | 6-(2,4-dimethylthiazol-5-yl)-4- fluoro-2-(6-(methyl(2,2,6,6- tetramethylpiperidin-4- yl)amino)pyridazin-3- yl)isoindolin-1-one |
| 292 | 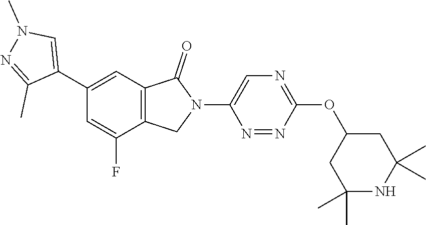 [SMILES: Cc1nn(C)cc1-c1cc(F)c2c(c1)C(=O)N(c1cnc(OC3CC(C)(C)NC(C)(C)C3)nn1)C2] | 6-(1,3-dimethyl-1H-pyrazol-4-yl)- 4-fluoro-2-(3-((2,2,6,6- tetramethylpiperidin-4-yl)oxy)- 1,2,4-triazin-6-yl)isoindolin-1-one |
| 293 | 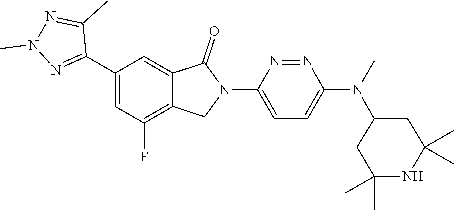 | 6-(2,5-dimethyl-2H-1,2,3-triazol- 4-yl)-4-fluoro-2-(6- (methyl(2,2,6,6- tetramethylpiperidin-4- yl)amino)pyridazin-3- yl)isoindolin-1-one |
| 308 | 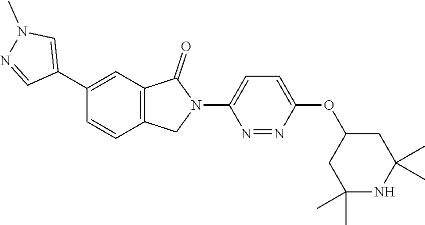 [SMILES: Cn1cc(-c2ccc3c(c2)C(=O)N(c2ccc(OC4CC(C)(C)NC(C)(C)C4)nn2)C3)cn1] | 6-(1-methyl-1H-pyrazol-4-yl)-2- (6-((2,2,6,6-tetramethylpiperidin- 4-yl)oxy)pyridazin-3- yl)isoindolin-1-one |
| 309 | 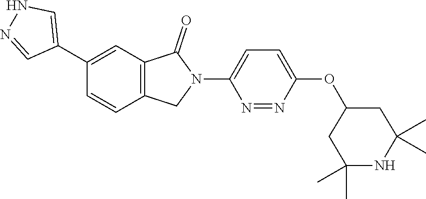 [SMILES: CC1(C)CC(Oc2ccc(N3Cc4ccc(-c5cn[nH]c5)cc4C3=O)nn2)CC(C)(C)N1] | 6-(1H-pyrazol-4-yl)-2-(6-((2,2,6,6- tetramethylpiperidin-4- yl)oxy)pyridazin-3-yl)isoindolin- 1-one |
| 310 | 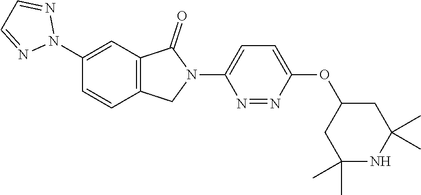 [SMILES: CC1(C)CC(Oc2ccc(N3Cc4ccc(-n5nccn5)cc4C3=O)nn2)CC(C)(C)N1] | 2-(6-((2,2,6,6- tetramethylpiperidin-4- yl)oxy)pyridazin-3-yl)-6-(2H- 1,2,3-triazol-2-yl)isoindolin-1-one |
| 311 | 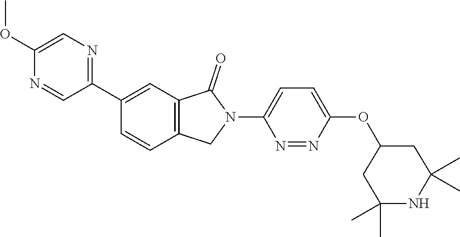 [SMILES: COc1cnc(-c2ccc3c(c2)C(=O)N(c2ccc(OC4CC(C)(C)NC(C)(C)C4)nn2)C3)cn1] | 6-(5-methoxypyrazin-2-yl)-2-(6- ((2,2,6,6-tetramethylpiperidin-4- yl)oxy)pyridazin-3-yl)isoindolin- 1-one |
| 312 | 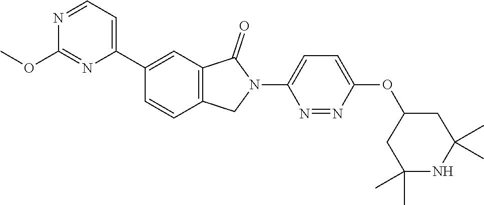 | 6-(2-methoxypyrimidin-4-yl)-2-(6- ((2,2,6,6-tetramethylpiperidin-4- yl)oxy)pyridazin-3-yl)isoindolin- 1-one |
| 313 | 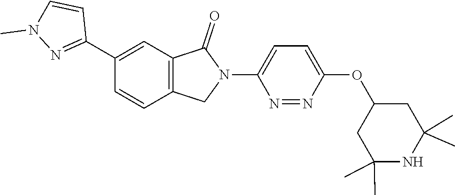 | 6-(1-methyl-1H-pyrazol-3-yl)-2- (6-((2,2,6,6-tetramethylpiperidin- 4-yl)oxy)pyridazin-3- yl)isoindolin-1-one |
| 314 | 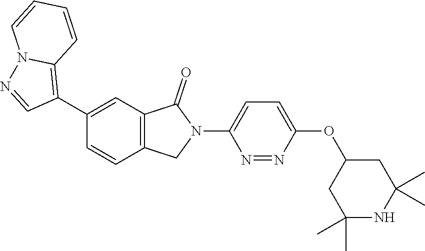 [SMILES: CC1(C)CC(Oc2ccc(N3Cc4ccc(-c5cnn6ccccc56)cc4C3=O)nn2)CC(C)(C)N1] | 6-(pyrazolo[1,5-a]pyridin-3-yl)-2- (6-((2,2,6,6-tetramethylpiperidin- 4-yl)oxy)pyridazin-3- yl)isoindolin-1-one |
| 315 | 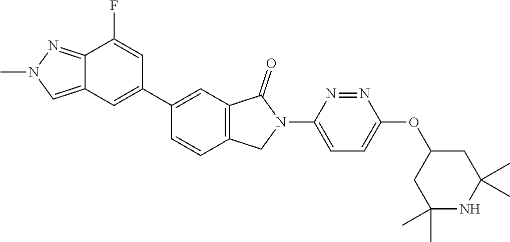 | 6-(7-fluoro-2-methyl-2H-indazol- 5-yl)-2-(6-((2,2,6,6- tetramethylpiperidin-4- yl)oxy)pyridazin-3-yl)isoindolin- 1-one |
| 316 | 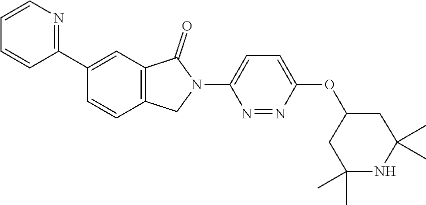 [SMILES: CC1(C)CC(Oc2ccc(N3Cc4ccc(-c5ccccn5)cc4C3=O)nn2)CC(C)(C)N1] | 6-(pyridin-2-yl)-2-(6-((2,2,6,6- tetramethylpiperidin-4- yl)oxy)pyridazin-3-yl)isoindolin- 1-one |
| 317 | 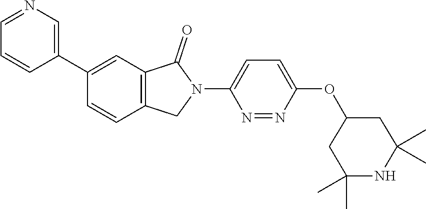 [SMILES: CC1(C)CC(Oc2ccc(N3Cc4ccc(-c5cccnc5)cc4C3=O)nn2)CC(C)(C)N1] | 6-(pyridin-3-yl)-2-(6-((2,2,6,6- tetramethylpiperidin-4- yl)oxy)pyridazin-3-yl)isoindolin- 1-one |
| 318 | 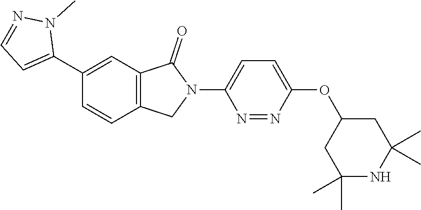 [SMILES: Cn1nccc1-c1ccc2c(c1)C(=O)N(c1ccc(OC3CC(C)(C)NC(C)(C)C3)nn1)C2] | 6-(1-methyl-1H-pyrazol-5-yl)-2- (6-((2,2,6,6-tetramethylpiperidin- 4-yl)oxy)pyridazin-3- yl)isoindolin-1-one |
| 319 | 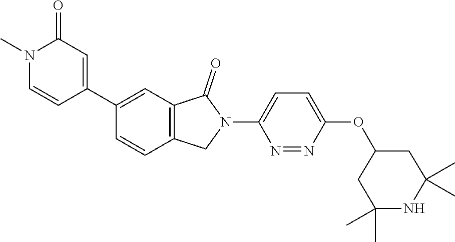 | 6-(1-methyl-2-oxo-1,2- dihydropyridin-4-yl)-2-(6- ((2,2,6,6-tetramethylpiperidin-4- yl)oxy)pyridazin-3-yl)isoindolin- 1-one |
| 320 | 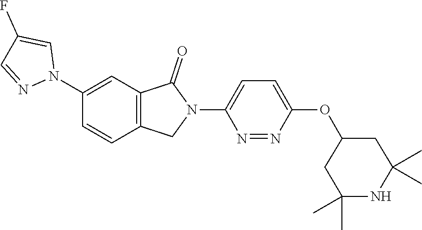 [SMILES: CC1(C)CC(Oc2ccc(N3Cc4ccc(-n5cc(F)cn5)cc4C3=O)nn2)CC(C)(C)N1] | 6-(4-fluoro-1H-pyrazol-1-yl)-2-(6- ((2,2,6,6-tetramethylpiperidin-4- yl)oxy)pyridazin-3-yl)isoindolin- 1-one |
| 321 | 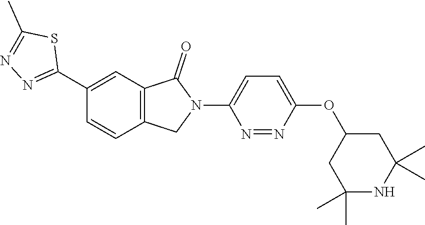 [SMILES: Cc1nnc(-c2ccc3c(c2)C(=O)N(c2ccc(OC4CC(C)(C)NC(C)(C)C4)nn2)C3)s1] | 6-(5-methyl-1,3,4-thiadiazol-2-yl)- 2-(6-((2,2,6,6- tetramethylpiperidin-4- yl)oxy)pyridazin-3-yl)isoindolin- 1-one |
| 322 | 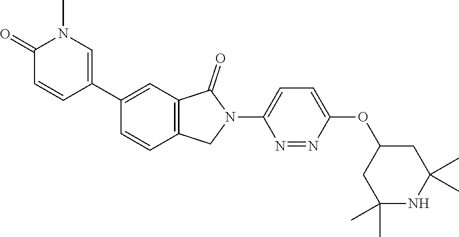 [SMILES: Cn1cc(-c2ccc3c(c2)C(=O)N(c2ccc(OC4CC(C)(C)NC(C)(C)C4)nn2)C3)ccc1=O] | 6-(1-methyl-6-oxo-1,6- dihydropyridin-3-yl)-2-(6- ((2,2,6,6-tetramethylpiperidin-4- yl)oxy)pyridazin-3-yl)isoindolin- 1-one |
| 323 | 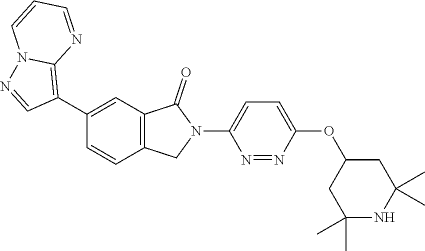 [SMILES: CC1(C)CC(Oc2ccc(N3Cc4ccc(-c5cnn6cccnc56)cc4C3=O)nn2)CC(C)(C)N1] | 6-(pyrazolo[1,5-a]pyrimidin-3-yl)- 2-(6-((2,2,6,6- tetramethylpiperidin-4- yl)oxy)pyridazin-3-yl)isoindolin- 1-one |
| 324 | 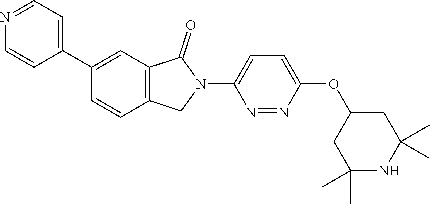 [SMILES: CC1(C)CC(Oc2ccc(N3Cc4ccc(-c5ccncc5)cc4C3=O)nn2)CC(C)(C)N1] | 6-(pyridin-4-yl)-2-(6-((2,2,6,6- tetramethylpiperidin-4- yl)oxy)pyridazin-3-yl)isoindolin- 1-one |
| 325 | 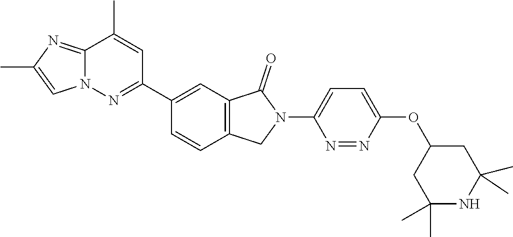 | 6-(2,8-dimethylimidazo[1,2- b]pyridazin-6-yl)-2-(6-((2,2,6,6- tetramethylpiperidin-4- yl)oxy)pyridazin-3-yl)isoindolin- 1-one |
| 326 | 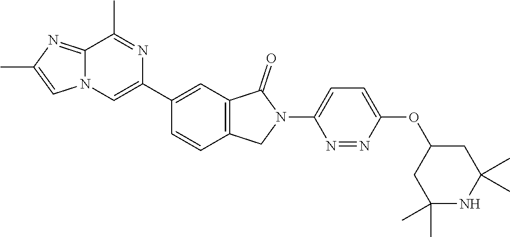 | 6-(2,8-dimethylimidazo[1,2- a]pyrazin-6-yl)-2-(6-((2,2,6,6- tetramethylpiperidin-4- yl)oxy)pyridazin-3-yl)isoindolin- 1-one |
| 327 | 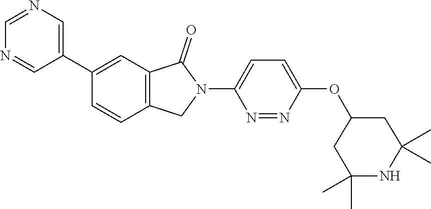 [SMILES: CC1(C)CC(Oc2ccc(N3Cc4ccc(-c5cncnc5)cc4C3=O)nn2)CC(C)(C)N1] | 6-(pyrimidin-5-yl)-2-(6-((2,2,6,6- tetramethylpiperidin-4- yl)oxy)pyridazin-3-yl)isoindolin- 1-one |
| 328 | 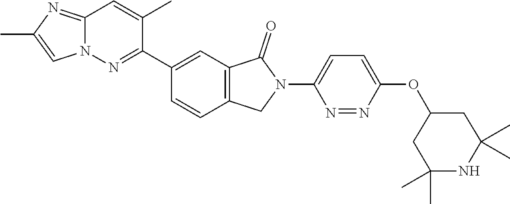 | 6-(2,7-dimethylimidazo[1,2- b]pyridazin-6-yl)-2-(6-((2,2,6,6- tetramethylpiperidin-4- yl)oxy)pyridazin-3-yl)isoindolin- 1-one |
| 329 | 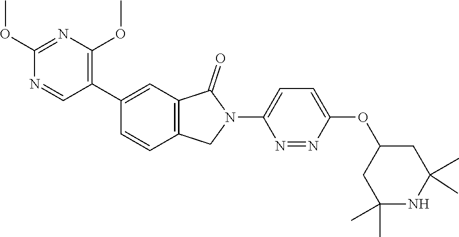 [SMILES: COc1ncc(-c2ccc3c(c2)C(=O)N(c2ccc(OC4CC(C)(C)NC(C)(C)C4)nn2)C3)c(OC)n1] | 6-(2,4-dimethoxypyrimidin-5-yl)- 2-(6-((2,2,6,6- tetramethylpiperidin-4- yl)oxy)pyridazin-3-yl)isoindolin- 1-one |
| 330 | 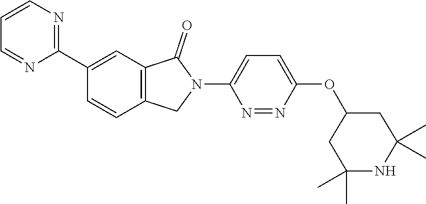 [SMILES: CC1(C)CC(Oc2ccc(N3Cc4ccc(-c5ncccn5)cc4C3=O)nn2)CC(C)(C)N1] | 6-(pyrimidin-2-yl)-2-(6-((2,2,6,6- tetramethylpiperidin-4- yl)oxy)pyridazin-3-yl)isoindolin- 1-one |
| 331 | 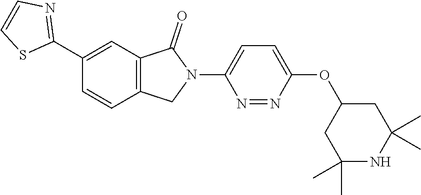 [SMILES: CC1(C)CC(Oc2ccc(N3Cc4ccc(-c5nccs5)cc4C3=O)nn2)CC(C)(C)N1] | 2-(6-((2,2,6,6- tetramethylpiperidin-4- yl)oxy)pyridazin-3-yl)-6-(thiazol- 2-yl)isoindolin-1-one |
| 332 | 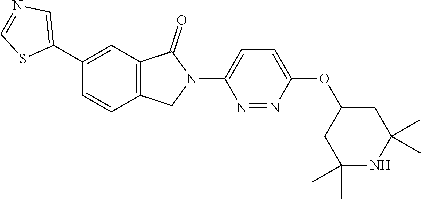 [SMILES: CC1(C)CC(Oc2ccc(N3Cc4ccc(-c5cncs5)cc4C3=O)nn2)CC(C)(C)N1] | 2-(6-((2,2,6,6- tetramethylpiperidin-4- yl)oxy)pyridazin-3-yl)-6-(thiazol- 5-yl)isoindolin-1-one |
| 333 | 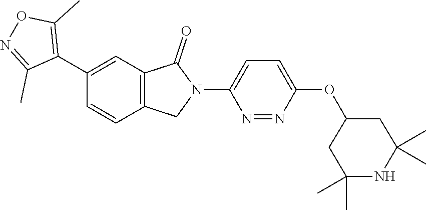 [SMILES: Cc1noc(C)c1-c1ccc2c(c1)C(=O)N(c1ccc(OC3CC(C)(C)NC(C)(C)C3)nn1)C2] | 6-(3,5-dimethylisoxazol-4-yl)-2- (6-((2,2,6,6-tetramethylpiperidin- 4-yl)oxy)pyridazin-3- yl)isoindolin-1-one |
| 334 | 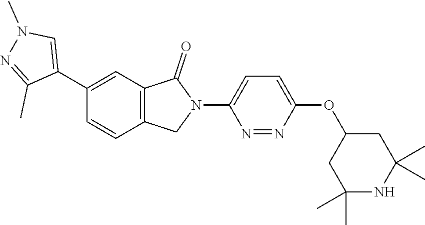 [SMILES: Cc1nn(C)cc1-c1ccc2c(c1)C(=O)N(c1ccc(OC3CC(C)(C)NC(C)(C)C3)nn1)C2] | 2-(6-((2,2,6,6- tetramethylpiperidin-4- yl)oxy)pyridazin-3-yl)-6-(1,3,5- trimethyl-1H-pyrazol-4- yl)isoindolin-1-one |
| 335 | 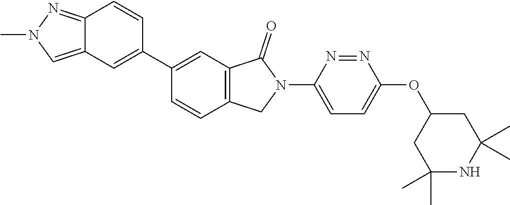 | 6-(2-methyl-2H-indazol-5-yl)-2- (6-((2,2,6,6-tetramethylpiperidin- 4-yl)oxy)pyridazin-3- yl)isoindolin-1-one |
| 336 | 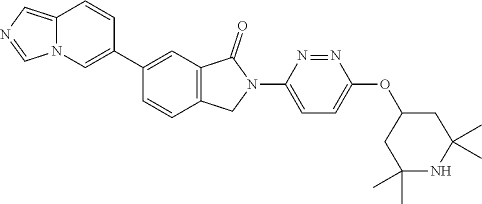 [SMILES: CC1(C)CC(Oc2ccc(N3Cc4ccc(-c5ccc6cncn6c5)cc4C3=O)nn2)CC(C)(C)N1] | 6-(imidazo[1,5-a]pyridin-6-yl)-2- (6-((2,2,6,6-tetramethylpiperidin- 4-yl)oxy)pyridazin-3- yl)isoindolin-1-one |
| 337 | 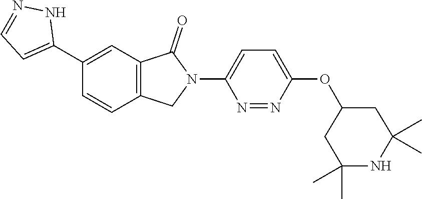 [SMILES: CC1(C)CC(Oc2ccc(N3Cc4ccc(-c5ccn[nH]5)cc4C3=O)nn2)CC(C)(C)N1] | 6-(1H-pyrazol-5-yl)-2-(6-((2,2,6,6- tetramethylpiperidin-4- yl)oxy)pyridazin-3-yl)isoindolin- 1-one |
| 338 | 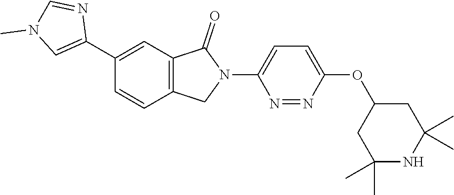 | 6-(1-methyl-1H-imidazol-4-yl)-2- (6-((2,2,6,6-tetramethylpiperidin- 4-yl)oxy)pyridazin-3- yl)isoindolin-1-one |
| 339 | 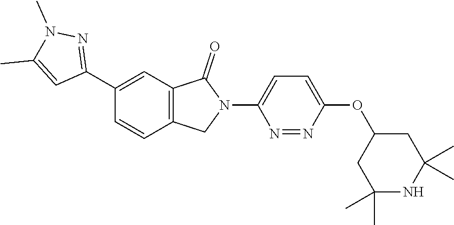 | 6-(1,5-dimethyl-1H-pyrazol-3-yl)- 2-(6-((2,2,6,6- tetramethylpiperidin-4- yl)oxy)pyridazin-3-yl)isoindolin- 1-one |
| 340 | 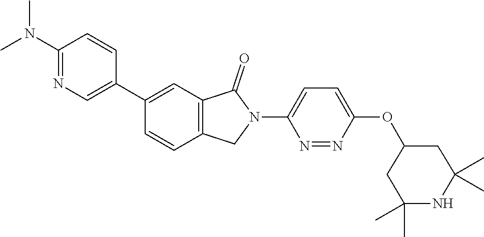 | 6-(6-(dimethylamino)pyridin-3- yl)-2-(6-((2,2,6,6- tetramethylpiperidin-4- yl)oxy)pyridazin-3-yl)isoindolin- 1-one |
| 341 | 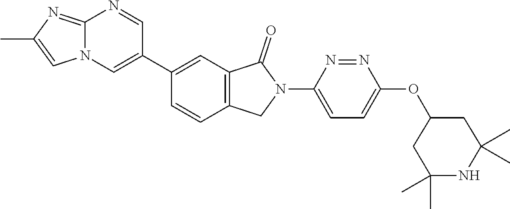 | 6-(2-methylimidazo[1,2- a]pyrimidin-6-yl)-2-(6-((2,2,6,6- tetramethylpiperidin-4- yl)oxy)pyridazin-3-yl)isoindolin- 1-one |
| 342 | 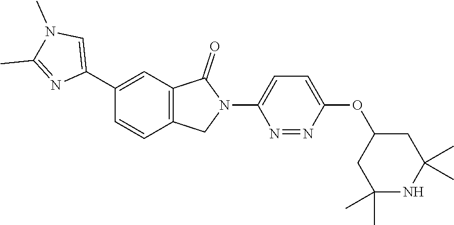 | 6-(1,2-dimethyl-1H-imidazol-4- yl)-2-(6-((2,2,6,6- tetramethylpiperidin-4- yl)oxy)pyridazin-3-yl)isoindolin- 1-one |
| 343 | 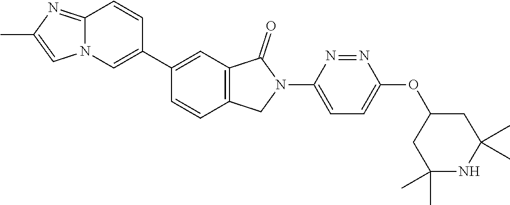 | 6-(2-methylimidazo[1,2-a]pyridin- 6-yl)-2-(6-((2,2,6,6- tetramethylpiperidin-4- yl)oxy)pyridazin-3-yl)isoindolin- 1-one |
| 346 | 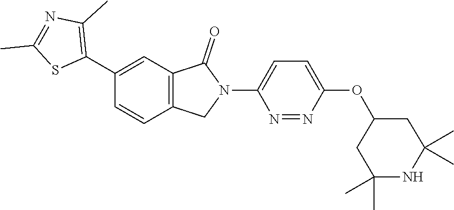 | 6-(2,4-dimethylthiazol-5-yl)-2-(6- ((2,2,6,6-tetramethylpiperidin-4- yl)oxy)pyridazin-3-yl)isoindolin- 1-one |
| 347 | 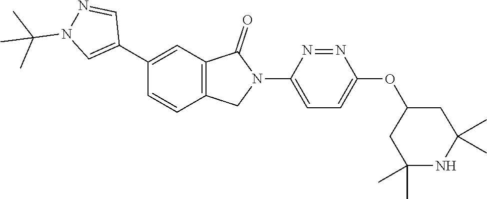 [SMILES: CC1(C)CC(Oc2ccc(N3Cc4ccc(-c5cnn(C(C)(C)C)c5)cc4C3=O)nn2)CC(C)(C)N1] | 6-(1-(tert-butyl)-1H-pyrazol-4-yl)- 2-(6-((2,2,6,6- tetramethylpiperidin-4- yl)oxy)pyridazin-3-yl)isoindolin- 1-one |
| 348 | 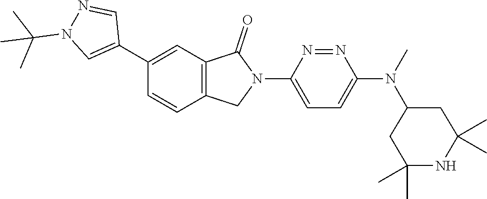 [SMILES: CN(c1ccc(N2Cc3ccc(-c4cnn(C(C)(C)C)c4)cc3C2=O)nn1)C1CC(C)(C)NC(C)(C)C1] | 6-(1-(tert-butyl)-1H-pyrazol-4-yl)- 2-(6-(methyl(2,2,6,6- tetramethylpiperidin-4- yl)amino)pyridazin-3- yl)isoindolin-1-one |
| 349 | 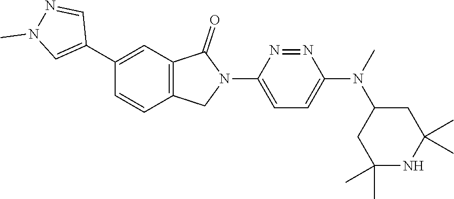 | 2-(6-(methyl(2,2,6,6- tetramethylpiperidin-4- yl)amino)pyridazin-3-yl)-6-(1- methyl-1H-pyrazol-4- yl)isoindolin-1-one |
| 350 | 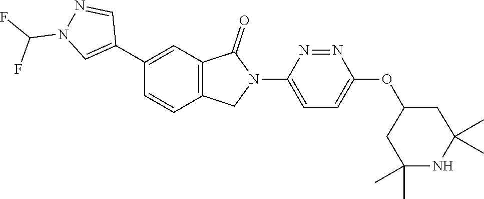 [SMILES: CC1(C)CC(Oc2ccc(N3Cc4ccc(-c5cnn(C(F)F)c5)cc4C3=O)nn2)CC(C)(C)N1] | 6-(1-(difluoromethyl)-1H-pyrazol- 4-yl)-2-(6-((2,2,6,6- tetramethylpiperidin-4- yl)oxy)pyridazin-3-yl)isoindolin- 1-one |
| 351 | 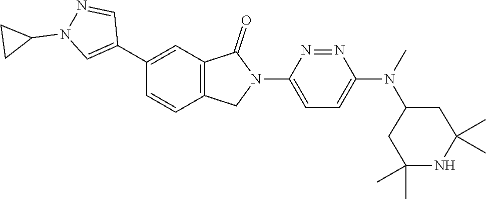 [SMILES: CN(c1ccc(N2Cc3ccc(-c4cnn(C5CC5)c4)cc3C2=O)nn1)C1CC(C)(C)NC(C)(C)C1] | 6-(1-cyclopropyl-1H-pyrazol-4- yl)-2-(6-(methyl(2,2,6,6- tetramethylpiperidin-4- yl)amino)pyridazin-3- yl)isoindolin-1-one |
| 352 | 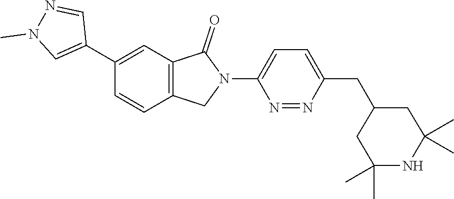 | 6-(1-methyl-1H-pyrazol-4-yl)-2- (6-((2,2,6,6-tetramethylpiperidin- 4-yl)methyl)pyridazin-3- yl)isoindolin-1-one |
| 353 | 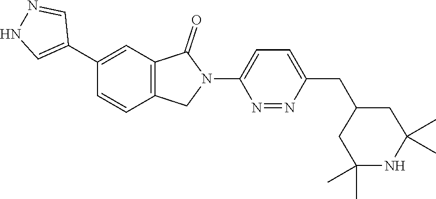 [SMILES: CC1(C)CC(Cc2ccc(N3Cc4ccc(-c5cn[nH]c5)cc4C3=O)nn2)CC(C)(C)N1] | 6-(1H-pyrazol-4-yl)-2-(6-((2,2,6,6- tetramethylpiperidin-4- yl)methyl)pyridazin-3- yl)isoindolin-1-one |
| 354 | 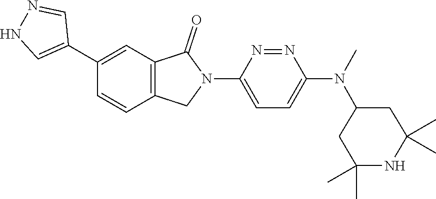 [SMILES: CN(c1ccc(N2Cc3ccc(-c4cn[nH]c4)cc3C2=O)nn1)C1CC(C)(C)NC(C)(C)C1] | 2-(6-(methyl(2,2,6,6- tetramethylpiperidin-4- yl)amino)pyridazin-3-yl)-6-(1H- pyrazol-4-yl)isoindolin-1-one |
| 355 | 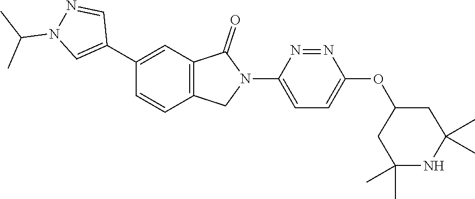 [SMILES: CC(C)n1cc(-c2ccc3c(c2)C(=O)N(c2ccc(OC4CC(C)(C)NC(C)(C)C4)nn2)C3)cn1] | 6-(1-isopropyl-1H-pyrazol-4-yl)-2- (6-((2,2,6,6-tetramethylpiperidin- 4-yl)oxy)pyridazin-3- yl)isoindolin-1-one |
| 356 | 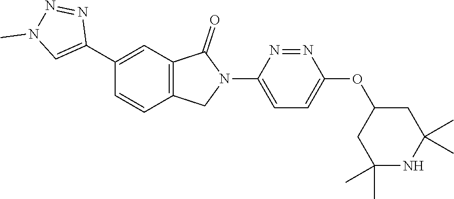 | 6-(1-methyl-1H-1,2,3-triazol-4- yl)-2-(6-((2,2,6,6- tetramethylpiperidin-4- yl)oxy)pyridazin-3-yl)isoindolin- 1-one |
| 357 | 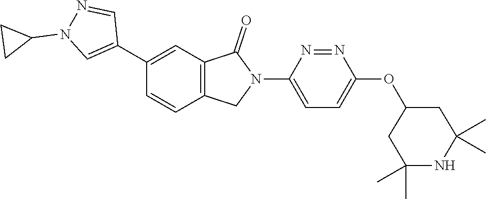 [SMILES: CC1(C)CC(Oc2ccc(N3Cc4ccc(-c5cnn(C6CC6)c5)cc4C3=O)nn2)CC(C)(C)N1] | 6-(1-cyclopropyl-1H-pyrazol-4- yl)-2-(6-((2,2,6,6- tetramethylpiperidin-4- yl)oxy)pyridazin-3-yl)isoindolin- 1-one |
| 358 | 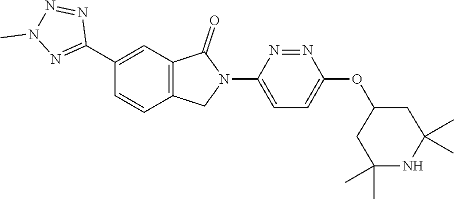 | 6-(2-methyl-2H-tetrazol-5-yl)-2- (6-((2,2,6,6-tetramethylpiperidin- 4-yl)oxy)pyridazin-3- yl)isoindolin-1-one |
| 359 | 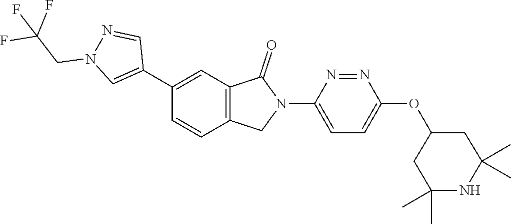 [SMILES: CC1(C)CC(Oc2ccc(N3Cc4ccc(-c5cnn(CC(F)(F)F)c5)cc4C3=O)nn2)CC(C)(C)N1] | 2-(6-((2,2,6,6- tetramethylpiperidin-4- yl)oxy)pyridazin-3-yl)-6-(1-(2,2,2- trifluoroethyl)-1H-pyrazol-4- yl)isoindolin-1-one |
| 360 | 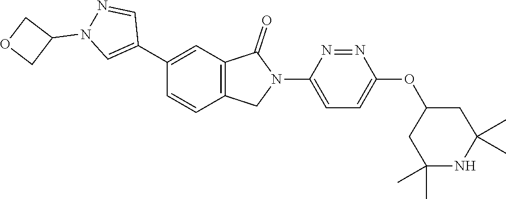 [SMILES: CC1(C)CC(Oc2ccc(N3Cc4ccc(-c5cnn(C6COC6)c5)cc4C3=O)nn2)CC(C)(C)N1] | 6-(1-(oxetan-3-yl)-1H-pyrazol-4- yl)-2-(6-((2,2,6,6- tetramethylpiperidin-4- yl)oxy)pyridazin-3-yl)isoindolin- 1-one |
| 362 | 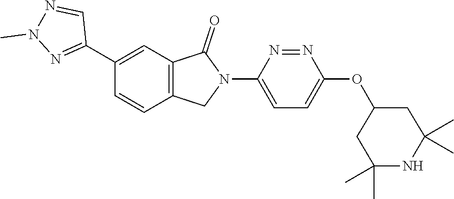 | 6-(2-methyl-2H-1,2,3-triazol-4- yl)-2-(6-((2,2,6,6- tetramethylpiperidin-4- yl)oxy)pyridazin-3-yl)isoindolin- 1-one |
| 363 | 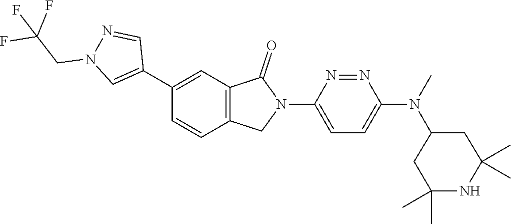 [SMILES: CN(c1ccc(N2Cc3ccc(-c4cnn(CC(F)(F)F)c4)cc3C2=O)nn1)C1CC(C)(C)NC(C)(C)C1] | 2-(6-(methyl(2,2,6,6- tetramethylpiperidin-4- yl)amino)pyridazin-3-yl)-6-(1- (2,2,2-trifluoroethyl)-1H-pyrazol- 4-yl)isoindolin-1-one |
| 364 | 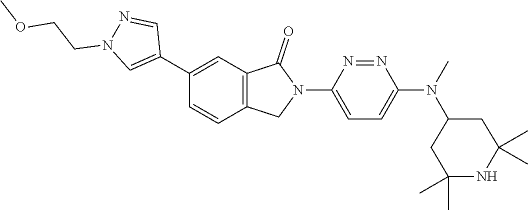 | 6-(1-(2-methoxyethyl)-1H- pyrazol-4-yl)-2-(6-(methyl(2,2,6,6- tetramethylpiperidin-4- yl)amino)pyridazin-3- yl)isoindolin-1-one |
| 365 | 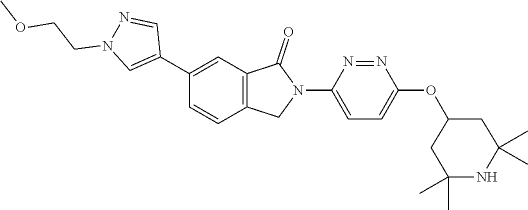 | 6-(1-(2-methoxyethyl)-1H- pyrazol-4-yl)-2-(6-((2,2,6,6- tetramethylpiperidin-4- yl)oxy)pyridazin-3-yl)isoindolin- 1-one |
| 368 | 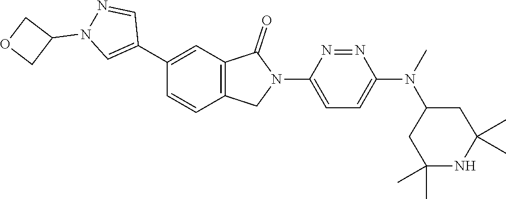 [SMILES: CN(c1ccc(N2Cc3ccc(-c4cnn(C5COC5)c4)cc3C2=O)nn1)C1CC(C)(C)NC(C)(C)C1] | 2-(6-(methyl(2,2,6,6- tetramethylpiperidin-4- yl)amino)pyridazin-3-yl)-6-(1- (oxetan-3-yl)-1H-pyrazol-4- yl)isoindolin-1-one |
| 369 | 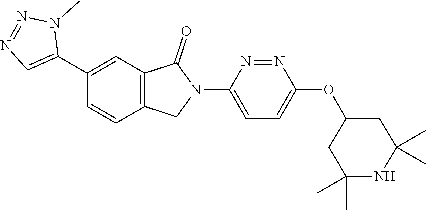 [SMILES: Cn1nncc1-c1ccc2c(c1)C(=O)N(c1ccc(OC3CC(C)(C)NC(C)(C)C3)nn1)C2] | 6-(1-methyl-1H-1,2,3-triazol-5- yl)-2-(6-((2,2,6,6- tetramethylpiperidin-4- yl)oxy)pyridazin-3-yl)isoindolin- 1-one |
| 370 | 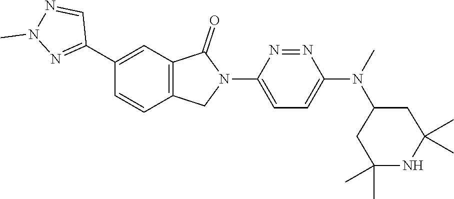 | 2-(6-(methyl(2,2,6,6- tetramethylpiperidin-4- yl)amino)pyridazin-3-yl)-6-(2- methyl-2H-1,2,3-triazol-4- yl)isoindolin-1-one |
| 371 | 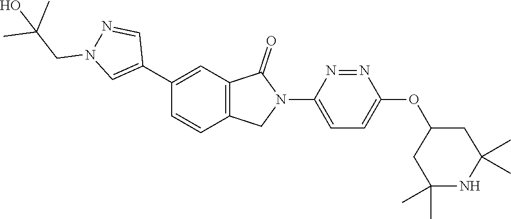 [SMILES: CC(C)(O)Cn1cc(-c2ccc3c(c2)C(=O)N(c2ccc(OC4CC(C)(C)NC(C)(C)C4)nn2)C3)cn1] | 6-(1-(2-hydroxy-2-methylpropyl)- 1H-pyrazol-4-yl)-2-(6-((2,2,6,6- tetramethylpiperidin-4- yl)oxy)pyridazin-3-yl)isoindolin- 1-one |
| 372 | 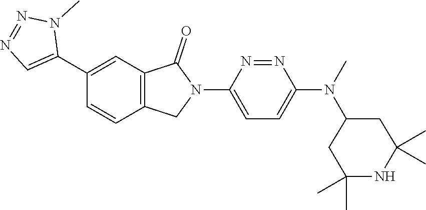 [SMILES: CN(c1ccc(N2Cc3ccc(-c4cnnn4C)cc3C2=O)nn1)C1CC(C)(C)NC(C)(C)C1] | 2-(6-(methyl(2,2,6,6- tetramethylpiperidin-4- yl)amino)pyridazin-3-yl)-6-(1- methyl-1H-1,2,3-triazol-5- yl)isoindolin-1-one |
| 373 | 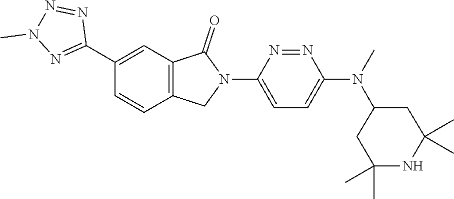 | 2-(6-(methyl(2,2,6,6- tetramethylpiperidin-4- yl)amino)pyridazin-3-yl)-6-(2- methyl-2H-tetrazol-5- yl)isoindolin-1-one |
| 375 | 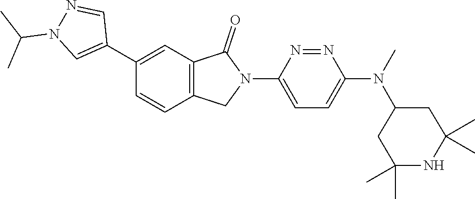 [SMILES: CC(C)n1cc(-c2ccc3c(c2)C(=O)N(c2ccc(N(C)C4CC(C)(C)NC(C)(C)C4)nn2)C3)cn1] | 6-(1-isopropyl-1 H-pyrazol-4-yl)-2- (6-(methyl(2,2,6,6- tetramethylpiperidin-4- yl)amino)pyridazin-3- yl)isoindolin-1-one |
| 378 | 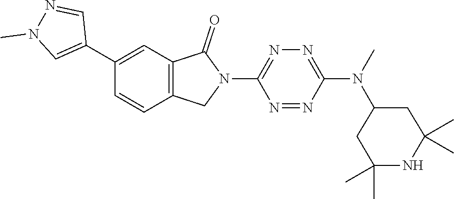 | 2-(6-(methyl(2,2,6,6- tetramethylpiperidin-4-yl)amino)- 1,2,4,5-tetrazin-3-yl)-6-(1-methyl- 1H-pyrazol-4-yl)isoindolin-1-one |
| 381 | 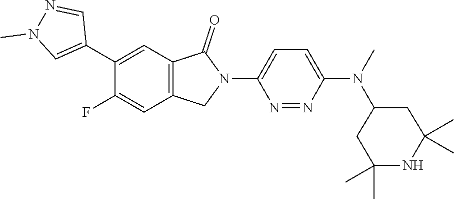 | 5-fluoro-2-(6-(methyl(2,2,6,6- tetramethylpiperidin-4- yl)amino)pyridazin-3-yl)-6-(1- methyl-1H-pyrazol-4- yl)isoindolin-1-one |
| 382 | 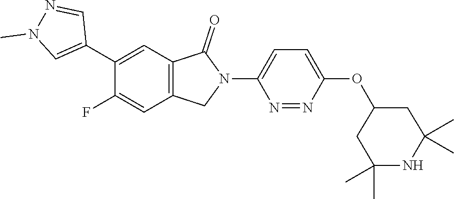 | 5-fluoro-6-(1-methyl-1H-pyrazol- 4-yl)-2-(6-((2,2,6,6- tetramethylpiperidin-4- yl)oxy)pyridazin-3-yl)isoindolin- 1-one |
| 383 | 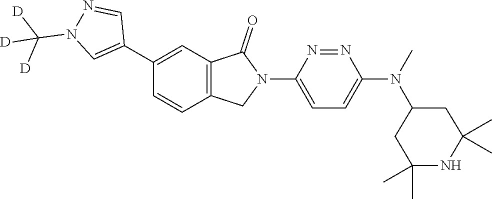 [SMILES: [2H]C([2H])([2H])n1cc(-c2ccc3c(c2)C(=O)N(c2ccc(N(C)C4CC(C)(C)NC(C)(C)C4)nn2)C3)cn1] | 2-(6-(methyl(2,2,6,6- tetramethylpiperidin-4- yl)amino)pyridazin-3-yl)-6-(1- (methyl-d3)-1H-pyrazol-4- yl)isoindolin-1-one |
| 385 | 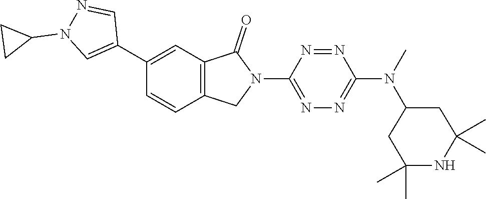 [SMILES: CN(c1nnc(N2Cc3ccc(-c4cnn(C5CC5)c4)cc3C2=O)nn1)C1CC(C)(C)NC(C)(C)C1] | 6-(1-cyclopropyl-1H-pyrazol-4- yl)-2-(6-(methyl(2,2,6,6- tetramethylpiperidin-4-yl)amino)- 1,2,4,5-tetrazin-3-yl)isoindolin-1- one |
| 392 | 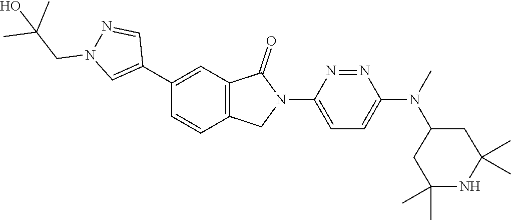 [SMILES: CN(c1ccc(N2Cc3ccc(-c4cnn(CC(C)(C)O)c4)cc3C2=O)nn1)C1CC(C)(C)NC(C)(C)C1] | 6-(1-(2-hydroxy-2-methylpropyl)- 1H-pyrazol-4-yl)-2-(6- (methyl(2,2,6,6- tetramethylpiperidin-4- yl)amino)pyridazin-3- yl)isoindolin-1-one |
| 403 | 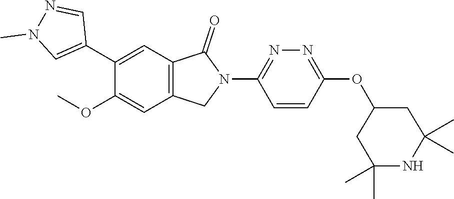 | 5-methoxy-6-(1-methyl-1H- pyrazol-4-yl)-2-(6-((2,2,6,6- tetramethylpiperidin-4- yl)oxy)pyridazin-3-yl)isoindolin- 1-one |
| 406 | 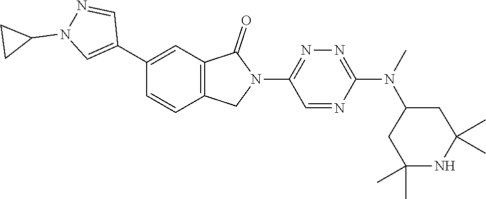 [SMILES: CN(c1ncc(N2Cc3ccc(-c4cnn(C5CC5)c4)cc3C2=O)nn1)C1CC(C)(C)NC(C)(C)C1] | 6-(1-cyclopropyl-1H-pyrazol-4- yl)-2-(3-(methyl(2,2,6,6- tetramethylpiperidin-4-yl)amino)- 1,2,4-triazin-6-yl)isoindolin-1-one |
| 410 | 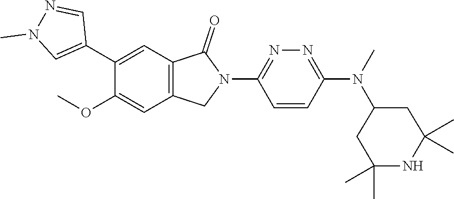 | 5-methoxy-2-(6-(methyl(2,2,6,6- tetramethylpiperidin-4- yl)amino)pyridazin-3-yl)-6-(1- methyl-1H-pyrazol-4- yl)isoindolin-1-one |
| 411 | 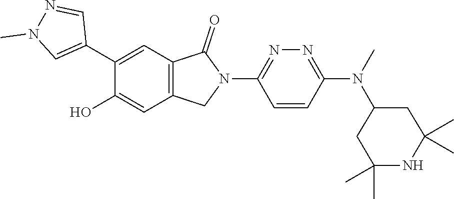 | 5-hydroxy-2-(6-(methyl(2,2,6,6- tetramethylpiperidin-4- yl)amino)pyridazin-3-yl)-6-(1- methyl-1H-pyrazol-4- yl)isoindolin-1-one |
| 417 | 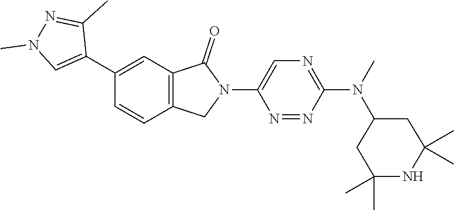 | 6-(1,3-dimethyl-1H-pyrazol-4-yl)- 2-(3-(methyl(2,2,6,6- tetramethylpiperidin-4-yl)amino)- 1,2,4-triazin-6-yl)isoindolin-1-one |
| 420 | 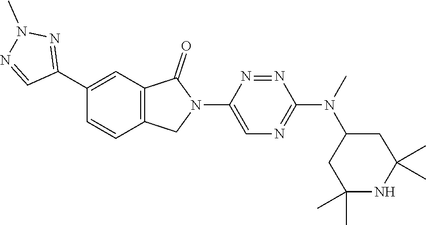 [SMILES: CN(c1ncc(N2Cc3ccc(-c4cnn(C)n4)cc3C2=O)nn1)C1CC(C)(C)NC(C)(C)C1] | 2-(3-(methyl(2,2,6,6- tetramethylpiperidin-4-yl)amino)- 1,2,4-triazin-6-yl)-6-(2-methyl- 2H-1,2,3-triazol-4-yl)isoindolin-1- one |
| 421 | 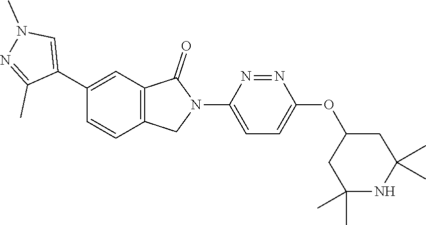 [SMILES: Cc1nn(C)cc1-c1ccc2c(c1)C(=O)N(c1ccc(OC3CC(C)(C)NC(C)(C)C3)nn1)C2] | 6-(1,3-dimethyl-1H-pyrazol-4-yl)- 2-(6-((2,2,6,6- tetramethylpiperidin-4- yl)oxy)pyridazin-3-yl)isoindolin- 1-one |
| 424 | 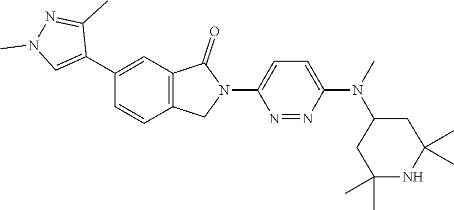 | 6-(1,3-dimethyl-1H-pyrazol-4-yl)- 2-(6-(methyl(2,2,6,6- tetramethylpiperidin-4- yl)amino)pyridazin-3- yl)isoindolin-1-one |
| 430 | 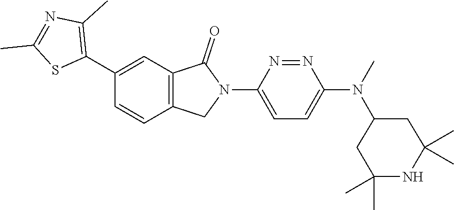 | 6-(2,4-dimethylthiazol-5-yl)-2-(6- (methyl(2,2,6,6- tetramethylpiperidin-4- yl)amino)pyridazin-3- yl)isoindolin-1-one |
| 431 | 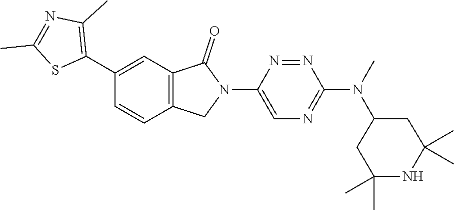 | 6-(2,4-dimethylthiazol-5-yl)-2-(3- (methyl(2,2,6,6- tetramethylpiperidin-4-yl)amino)- 1,2,4-triazin-6-yl)isoindolin-1-one |
| 433 | 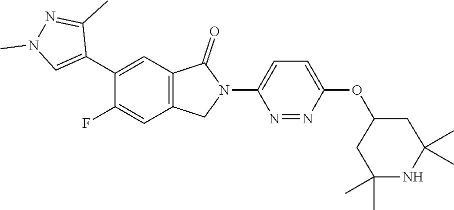 | 6-(1,3-dimethyl-1H-pyrazol-4-yl)- 5-fluoro-2-(6-((2,2,6,6- tetramethylpiperidin-4- yl)oxy)pyridazin-3-yl)isoindolin- 1-one |
| 435 | 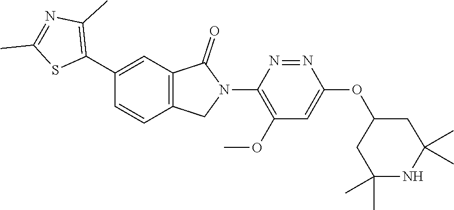 | 6-(2,4-dimethylthiazol-5-yl)-2-(4- methoxy-6-((2,2,6,6- tetramethylpiperidin-4- yl)oxy)pyridazin-3-yl)isoindolin- 1-one |
| 438 | 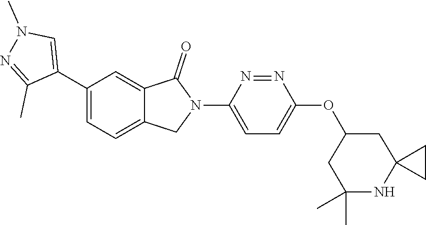 [SMILES: Cc1nn(C)cc1-c1ccc2c(c1)C(=O)N(c1ccc(OC3CC(C)(C)NC4(CC4)C3)nn1)C2] | 6-(1,3-dimethyl-1H-pyrazol-4-yl)- 2-(6-((5,5-dimethyl-4- azaspiro[2.5]octan-7- yl)oxy)pyridazin-3-yl)isoindolin- 1-one |
| 441 | 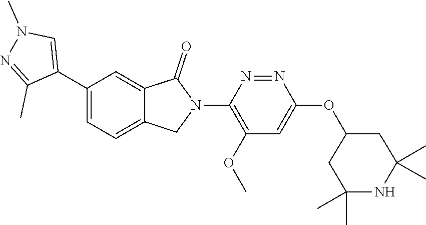 [SMILES: COc1cc(OC2CC(C)(C)NC(C)(C)C2)nnc1N1Cc2ccc(-c3cn(C)nc3C)cc2C1=O] | 6-(1,3-dimethyl-1H-pyrazol-4-yl)- 2-(4-methoxy-6-((2,2,6,6- tetramethylpiperidin-4- yl)oxy)pyridazin-3-yl)isoindolin- 1-one |
| 442 | 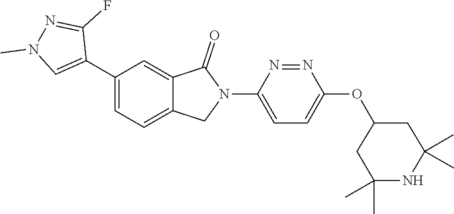 | 6-(3-fluoro-1-methyl-1H-pyrazol- 4-yl)-2-(6-((2,2,6,6- tetramethylpiperidin-4- yl)oxy)pyridazin-3-yl)isoindolin- 1-one |
| 443 | 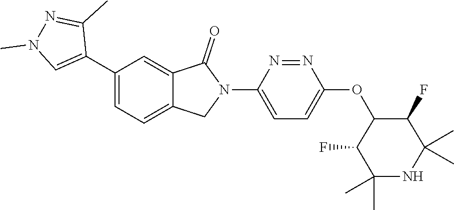 | 2-(6-(((3S,5S)-3,5-difluoro- 2,2,6,6-tetramethylpiperidin-4- yl)oxy)pyridazin-3-yl)-6-(1,3- dimethyl-1H-pyrazol-4- yl)isoindolin-1-one |
| 444 | 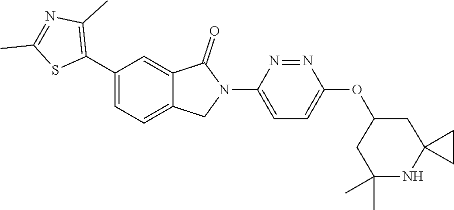 [SMILES: Cc1nc(C)c(-c2ccc3c(c2)C(=O)N(c2ccc(OC4CC(C)(C)NC5(CC5)C4)nn2)C3)s1] | 2-(6-((5,5-dimethyl-4- azaspiro[2.5]octan-7- yl)oxy)pyridazin-3-yl)-6-(2,4- dimethylthiazol-5-yl)isoindolin-1- one |
| 448 | 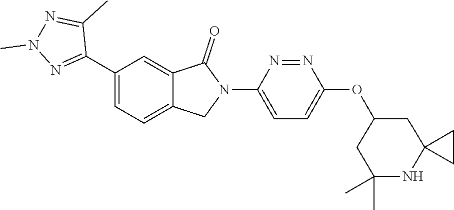 [SMILES: Cc1nn(C)nc1-c1ccc2c(c1)C(=O)N(c1ccc(OC3CC(C)(C)NC4(CC4)C3)nn1)C2] | 6-(2,5-dimethyl-2H-1,2,3-triazol- 4-yl)-2-(6-((5,5-dimethyl-4- azaspiro[2.5]octan-7- yl)oxy)pyridazin-3-yl)isoindolin- 1-one |
| 449 | 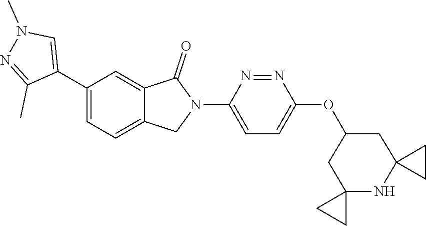 [SMILES: Cc1nn(C)cc1-c1ccc2c(c1)C(=O)N(c1ccc(OC3CC4(CC4)NC4(CC4)C3)nn1)C2] | 2-(6-((4- azadispiro[2.1.2⁵.3³]decan-9- yl)oxy)pyridazin-3-yl)-6-(1,3- dimethyl-1H-pyrazol-4- yl)isoindolin-1-one |
| 451 | 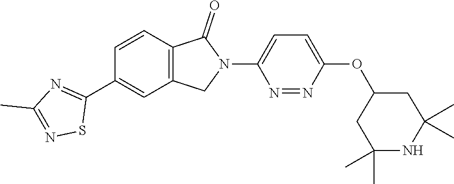 | 5-(3-methyl-1,2,4-thiadiazol-5-yl)- 2-(6-((2,2,6,6- tetramethylpiperidin-4- yl)oxy)pyridazin-3-yl)isoindolin- 1-one |
| 455 | 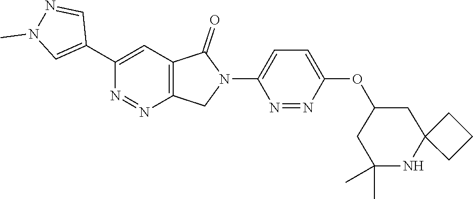 [SMILES: Cn1cc(-c2cc3c(nn2)CN(c2ccc(OC4CC(C)(C)NC5(CCC5)C4)nn2)C3=O)cn1] | 6-(6-((6,6-dimethyl-5- azaspiro[3.5]nonan-8- yl)oxy)pyridazin-3-yl)-3-(1- methyl-1H-pyrazol-4-yl)-6,7- dihydro-5H-pyrrolo[3,4- c]pyridazin-5-one |
| 457 | 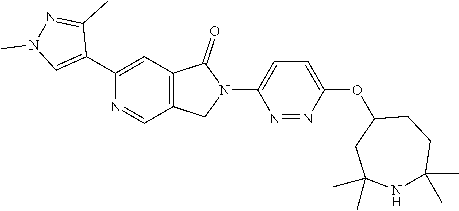 | 6-(1,3-dimethyl-1H-pyrazol-4-yl)- 2-(6-((2,2,7,7-tetramethylazepan- 4-yl)oxy)pyridazin-3-yl)-2,3- dihydro-1H-pyrrolo[3,4-c]pyridin- 1-one |
| 459 | 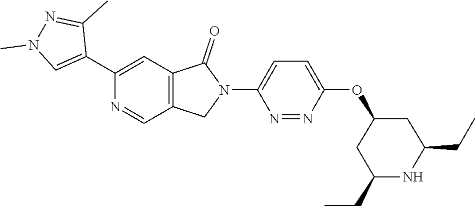 [SMILES: CC[C@@H]1C[C@H](Oc2ccc(N3Cc4cnc(-c5cn(C)nc5C)cc4C3=O)nn2)C[C@H](CC)N1] | 2-(6-(((2S,4r,6R)-2,6- diethylpiperidin-4- yl)oxy)pyridazin-3-yl)-6-(1,3- dimethyl-1H-pyrazol-4-yl)-2,3- dihydro-1H-pyrrolo[3,4-c]pyridin- 1-one |
| 461 | 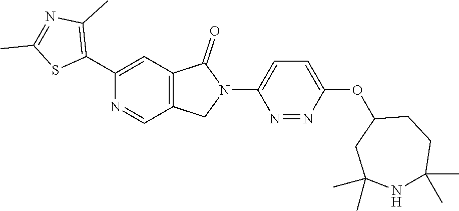 | 6-(2,4-dimethylthiazol-5-yl)-2-(6- ((2,2,7,7-tetramethylazepan-4- yl)oxy)pyridazin-3-yl)-2,3- dihydro-1H-pyrrolo[3,4-c]pyridin- 1-one |
| 462 | 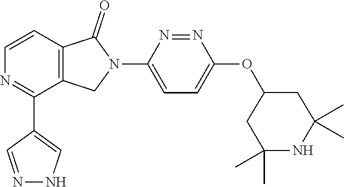 [SMILES: CC1(C)CC(Oc2ccc(N3Cc4c(ccnc4-c4cn[nH]c4)C3=O)nn2)CC(C)(C)N1] | 4-(1H-pyrazol-4-yl)-2-(6-((2,2,6,6- tetramethylpiperidin-4- yl)oxy)pyridazin-3-yl)-2,3- dihydro-1H-pyrrolo[3,4-c]pyridin- 1-one |
| 463 | 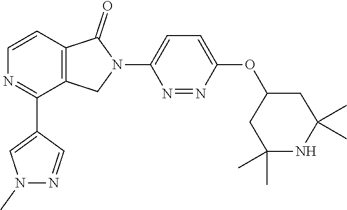 | 4-(1-methyl-1H-pyrazol-4-yl)-2- (6-((2,2,6,6-tetramethylpiperidin- 4-yl)oxy)pyridazin-3-yl)-2,3- dihydro-1H-pyrrolo[3,4-c]pyridin- 1-one |
| 464 | 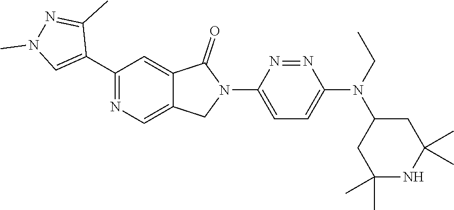 | 6-(1,3-dimethyl-1H-pyrazol-4-yl)- 2-(6-(ethyl(2,2,6,6- tetramethylpiperidin-4- yl)amino)pyridazin-3-yl)-2,3- dihydro-1H-pyrrolo[3,4-c]pyridin- 1-one |
| 465 | 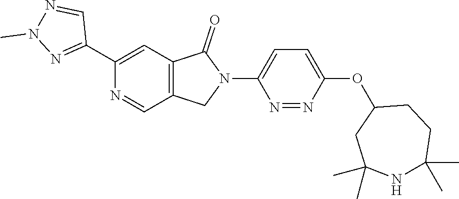 | 6-(2-methyl-2H-1,2,3-triazol-4- yl)-2-(6-((2,2,7,7- tetramethylazepan-4- yl)oxy)pyridazin-3-yl)-2,3- dihydro-1H-pyrrolo[3,4-c]pyridin- 1-one |
| 466 | 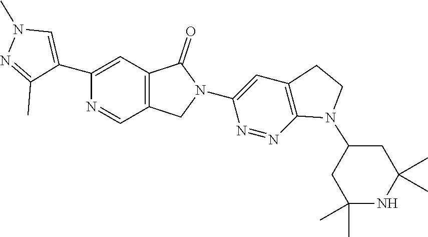 | 6-(1,3-dimethyl-1H-pyrazol-4-yl)- 2-(7-(2,2,6,6-tetramethylpiperidin- 4-yl)-6,7-dihydro-5H-pyrrolo[2,3- c]pyridazin-3-yl)-2,3-dihydro-1H- pyrrolo[3,4-c]pyridin-1-one |
| 468 | 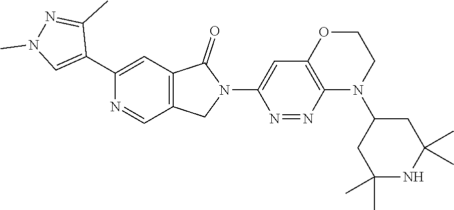 | 6-(1,3-dimethyl-1H-pyrazol-4-yl)- 2-(8-(2,2,6,6-tetramethylpiperidin- 4-yl)-7,8-dihydro-6H- pyridazino[4,3-b][1,4]oxazin-3- yl)-2,3-dihydro-1H-pyrrolo[3,4- c]pyridin-1-one |
| 469 | 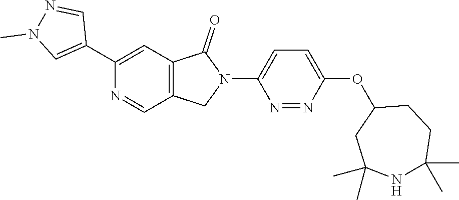 | 6-(1-methyl-1H-pyrazol-4-yl)-2- (6-((2,2,7,7-tetramethylazepan-4- yl)oxy)pyridazin-3-yl)-2,3- dihydro-1H-pyrrolo[3,4-c]pyridin- 1-one |
| 471 | 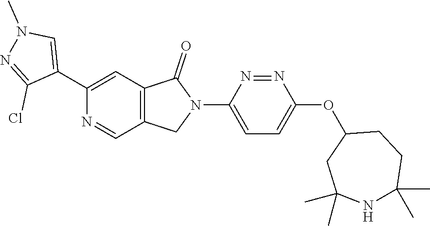 [SMILES: Cn1cc(-c2cc3c(cn2)CN(c2ccc(OC4CCC(C)(C)NC(C)(C)C4)nn2)C3=O)c(Cl)n1] | 6-(3-chloro-1-methyl-1H-pyrazol- 4-yl)-2-(6-((2,2,7,7- tetramethylazepan-4- yl)oxy)pyridazin-3-yl)-2,3- dihydro-1H-pyrrolo[3,4-c]pyridin- 1-one |
| 472 | 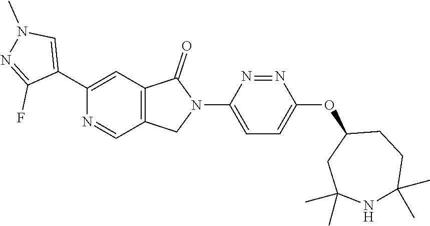 [SMILES: Cn1cc(-c2cc3c(cn2)CN(c2ccc(O[C@H]4CCC(C)(C)NC(C)(C)C4)nn2)C3=O)c(F)n1] | 6-(3-fluoro-1-methyl-1H-pyrazol- 4-yl)-2-(6-((2,2,7,7- tetramethylazepan-4- yl)oxy)pyridazin-3-yl)-2,3- dihydro-1H-pyrrolo[3,4-c]pyridin- 1-one |
| 473 | 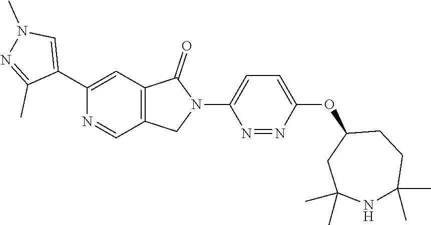 [SMILES: Cc1nn(C)cc1-c1cc2c(cn1)CN(c1ccc(O[C@H]3CCC(C)(C)NC(C)(C)C3)nn1)C2=O] | (S)-6-(1,3-dimethyl-1H-pyrazol-4- yl)-2-(6-((2,2,7,7- tetramethylazepan-4- yl)oxy)pyridazin-3-yl)-2,3- dihydro-1H-pyrrolo[3,4-c]pyridin- 1-one |
| 474 | 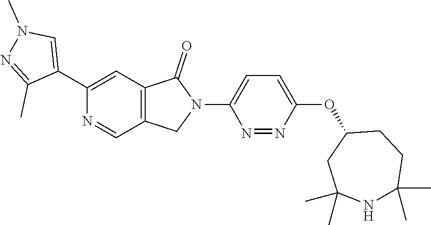 [SMILES: Cc1nn(C)cc1-c1cc2c(cn1)CN(c1ccc(O[C@@H]3CCC(C)(C)NC(C)(C)C3)nn1)C2=O] | (R)-6-(1,3-dimethyl-1H-pyrazol-4- yl)-2-(6-((2,2,7,7- tetramethylazepan-4- yl)oxy)pyridazin-3-yl)-2,3- dihydro-1H-pyrrolo[3,4-c]pyridin- 1-one |
| 475 | 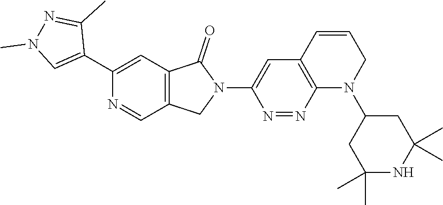 | 6-(1,3-dimethyl-1H-pyrazol-4-yl)- 2-(8-(2,2,6,6-tetramethylpiperidin- 4-yl)-7,8-dihydropyrido[2,3- c]pyridazin-3-yl)-2,3-dihydro-1H- pyrrolo[3,4-c]pyridin-1-one |
| 476 | 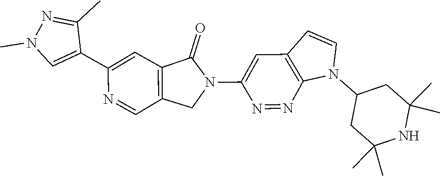 | 6-(1,3-dimethyl-1H-pyrazol-4-yl)- 2-(7-(2,2,6,6-tetramethylpiperidin- 4-yl)-7H-pyrrolo[2,3-c]pyridazin- 3-yl)-2,3-dihydro-1H-pyrrolo[3,4- c]pyridin-1-one |
| 479 | 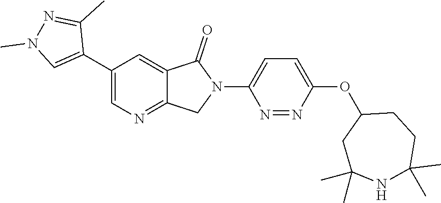 | 3-(1,3-dimethyl-1H-pyrazol-4-yl)- 6-(6-((2,2,7,7-tetramethylazepan- 4-yl)oxy)pyridazin-3-yl)-6,7- dihydro-5H-pyrrolo[3,4-b]pyridin- 5-one |
| 481 | 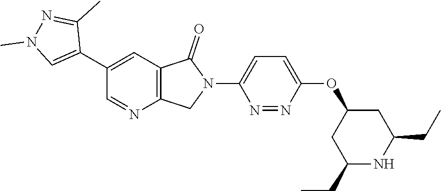 [SMILES: CC[C@@H]1C[C@H](Oc2ccc(N3Cc4ncc(-c5cn(C)nc5C)cc4C3=O)nn2)C[C@H](CC)N1] | 6-(6-(((2S,4r,6R)-2,6- diethylpiperidin-4- yl)oxy)pyridazin-3-yl)-3-(1,3- dimethyl-1H-pyrazol-4-yl)-6,7- dihydro-5H-pyrrolo[3,4-b]pyridin- 5-one |
| 482 | 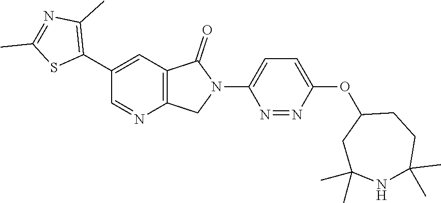 | 3-(2,4-dimethylthiazol-5-yl)-6-(6- ((2,2,7,7-tetramethylazepan-4- yl)oxy)pyridazin-3-yl)-6,7- dihydro-5H-pyrrolo[3,4-b]pyridin- 5-one |
| 483 | 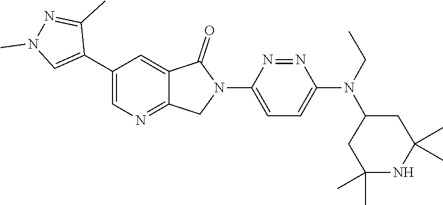 | 3-(1,3-dimethyl-1H-pyrazol-4-yl)- 6-(6-(ethyl(2,2,6,6- tetramethylpiperidin-4- yl)amino)pyridazin-3-yl)-6,7- dihydro-5H-pyrrolo[3,4-b]pyridin- 5-one |
| 484 | 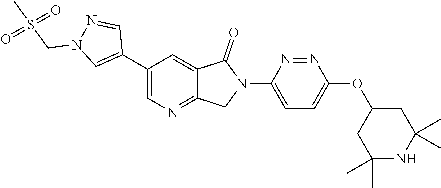 [SMILES: CC1(C)CC(Oc2ccc(N3Cc4ncc(-c5cnn(CS(C)(=O)=O)c5)cc4C3=O)nn2)CC(C)(C)N1] | 3-(1-((methylsulfonyl)methyl)-1H- pyrazol-4-yl)-6-(6-((2,2,6,6- tetramethylpiperidin-4- yl)oxy)pyridazin-3-yl)-6,7- dihydro-5H-pyrrolo[3,4-b]pyridin- 5-one |
| 485 | 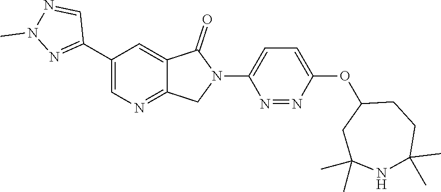 | 3-(2-methyl-2H-1,2,3-triazol-4- yl)-6-(6-((2,2,7,7- tetramethylazepan-4- yl)oxy)pyridazin-3-yl)-6,7- dihydro-5H-pyrrolo[3,4-b]pyridin- 5-one |
| 486 | 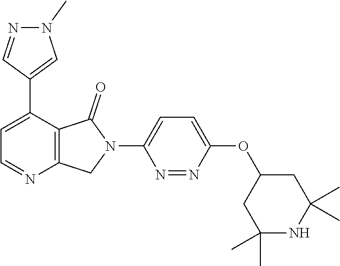 [SMILES: Cn1cc(-c2ccnc3c2C(=O)N(c2ccc(OC4CC(C)(C)NC(C)(C)C4)nn2)C3)cn1] | 4-(1-methyl-1H-pyrazol-4-yl)-6- (6-((2,2,6,6-tetramethylpiperidin- 4-yl)oxy)pyridazin-3-yl)-6,7- dihydro-5H-pyrrolo[3,4-b]pyridin- 5-one |
| 487 | 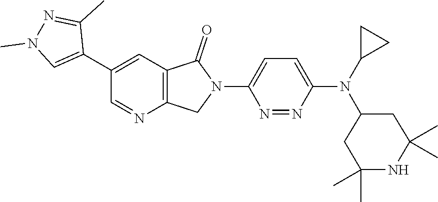 | 6-(6-(cyclopropyl(2,2,6,6- tetramethylpiperidin-4- yl)amino)pyridazin-3-yl)-3-(1,3- dimethyl-1H-pyrazol-4-yl)-6,7- dihydro-5H-pyrrolo[3,4-b]pyridin- 5-one |
| 488 | 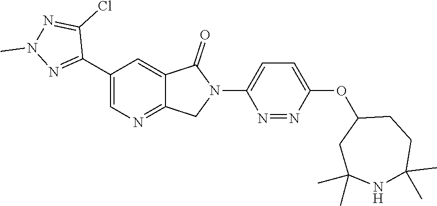 | 3-(5-chloro-2-methyl-2H-1,2,3- triazol-4-yl)-6-(6-((2,2,7,7- tetramethylazepan-4- yl)oxy)pyridazin-3-yl)-6,7- dihydro-5H-pyrrolo[3,4-b]pyridin- 5-one |
| 489 | 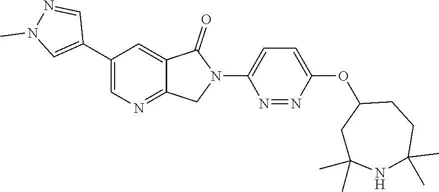 | 3-(1-methyl-1H-pyrazol-4-yl)-6- (6-((2,2,7,7-tetramethylazepan-4- yl)oxy)pyridazin-3-yl)-6,7- dihydro-5H-pyrrolo[3,4-b]pyridin- 5-one |
| 491 | 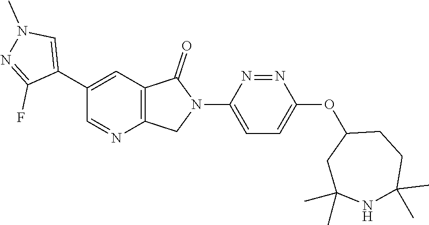 [SMILES: Cn1cc(-c2cnc3c(c2)C(=O)N(c2ccc(OC4CCC(C)(C)NC(C)(C)C4)nn2)C3)c(F)n1] | 3-(3-fluoro-1-methyl-1H-pyrazol- 4-yl)-6-(6-((2,2,7,7- tetramethylazepan-4- yl)oxy)pyridazin-3-yl)-6,7- dihydro-5H-pyrrolo[3,4-b]pyridin- 5-one |
| 492 | 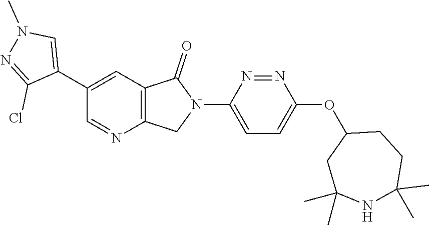 [SMILES: Cn1cc(-c2cnc3c(c2)C(=O)N(c2ccc(OC4CCC(C)(C)NC(C)(C)C4)nn2)C3)c(Cl)n1] | 3-(3-chloro-1-methyl-1H-pyrazol- 4-yl)-6-(6-((2,2,7,7- tetramethylazepan-4- yl)oxy)pyridazin-3-yl)-6,7- dihydro-5H-pyrrolo[3,4-b]pyridin- 5-one |
| 493 | 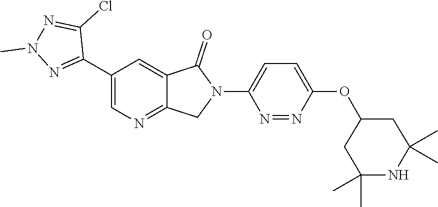 | 3-(5-chloro-2-methyl-2H-1,2,3- triazol-4-yl)-6-(6-((2,2,6,6- tetramethylpiperidin-4- yl)oxy)pyridazin-3-yl)-6,7- dihydro-5H-pyrrolo[3,4-b]pyridin- 5-one |
| 494 | 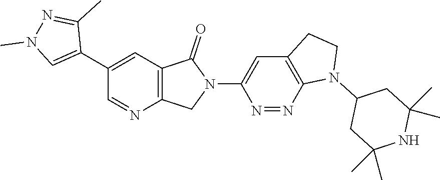 | 3-(1,3-dimethyl-1H-pyrazol-4-yl)- 6-(7-(2,2,6,6-tetramethylpiperidin- 4-yl)-6,7-dihydro-5H-pyrrolo[2,3- c]pyridazin-3-yl)-6,7-dihydro-5H- pyrrolo[3,4-b]pyridin-5-one |
| 497 | 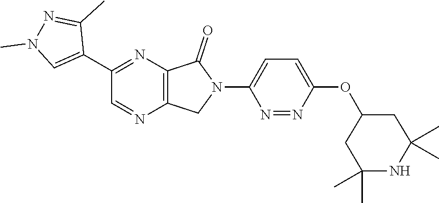 | 3-(1,3-dimethyl-1H-pyrazol-4-yl)- 6-(6-((2,2,6,6- tetramethylpiperidin-4- yl)oxy)pyridazin-3-yl)-6,7- dihydro-5H-pyrrolo[3,4-b]pyrazin- 5-one |
| 499 | 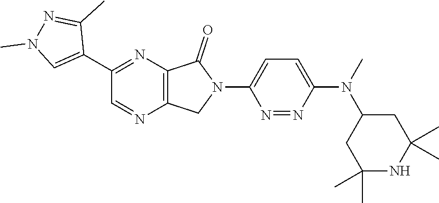 | 3-(1,3-dimethyl-1H-pyrazol-4-yl)- 6-(6-(methyl(2,2,6,6- tetramethylpiperidin-4- yl)amino)pyridazin-3-yl)-6,7- dihydro-5H-pyrrolo[3,4-b]pyrazin- 5-one |
| 500 | 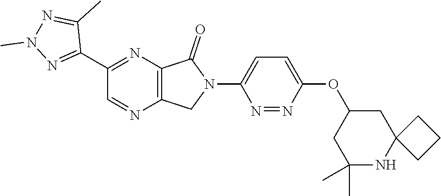 [SMILES: Cc1nn(C)nc1-c1cnc2c(n1)C(=O)N(c1ccc(OC3CC(C)(C)NC4(CCC4)C3)nn1)C2] | 3-(2,5-dimethyl-2H-1,2,3-triazol- 4-yl)-6-(6-((6,6-dimethyl-5- azaspiro[3.5]nonan-8- yl)oxy)pyridazin-3-yl)-6,7- dihydro-5H-pyrrolo[3,4-b]pyrazin- 5-one |
| 504 | 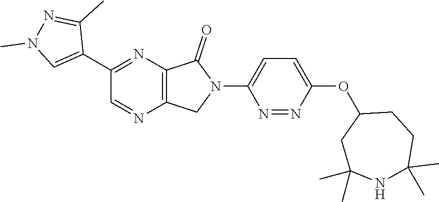 | 3-(1,3-dimethyl-1H-pyrazol-4-yl)- 6-(6-((2,2,7,7-tetramethylazepan- 4-yl)oxy)pyridazin-3-yl)-6,7- dihydro-5H-pyrrolo[3,4-b]pyrazin- 5-one |
| 505 | 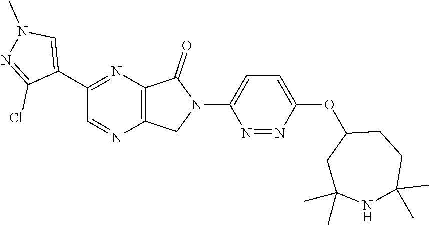 [SMILES: Cn1cc(-c2cnc3c(n2)C(=O)N(c2ccc(OC4CCC(C)(C)NC(C)(C)C4)nn2)C3)c(Cl)n1] | 3-(3-chloro-1-methyl-1H-pyrazol- 4-yl)-6-(6-((2,2,7,7- tetramethylazepan-4- yl)oxy)pyridazin-3-yl)-6,7- dihydro-5H-pyrrolo[3,4-b]pyrazin- 5-one |
| 506 | 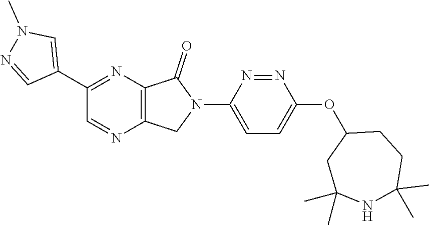 [SMILES: Cn1cc(-c2cnc3c(n2)C(=O)N(c2ccc(OC4CCC(C)(C)NC(C)(C)C4)nn2)C3)cn1] | 3-(1-methyl-1H-pyrazol-4-yl)-6- (6-((2,2,7,7-tetramethylazepan-4- yl)oxy)pyridazin-3-yl)-6,7- dihydro-5H-pyrrolo[3,4-b]pyrazin- 5-one |
| 507 | 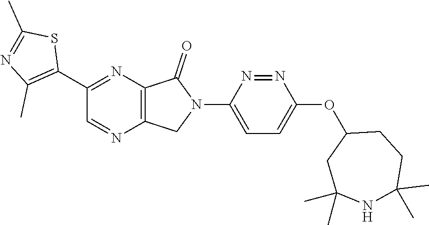 [SMILES: Cc1nc(C)c(-c2cnc3c(n2)C(=O)N(c2ccc(OC4CCC(C)(C)NC(C)(C)C4)nn2)C3)s1] | 3-(2,4-dimethylthiazol-5-yl)-6-(6- ((2,2,7,7-tetramethylazepan-4- yl)oxy)pyridazin-3-yl)-6,7- dihydro-5H-pyrrolo[3,4-b]pyrazin- 5-one |
| 508 | 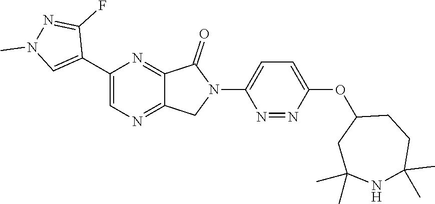 | 3-(3-fluoro-1-methyl-1H-pyrazol- 4-yl)-6-(6-((2,2,7,7- tetramethylazepan-4- yl)oxy)pyridazin-3-yl)-6,7- dihydro-5H-pyrrolo[3,4-b]pyrazin- 5-one |
| 509 | 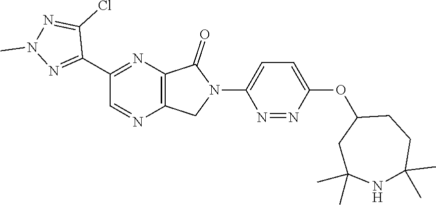 | 3-(5-chloro-2-methyl-2H-1,2,3- triazol-4-yl)-6-(6-((2,2,7,7- tetramethylazepan-4- yl)oxy)pyridazin-3-yl)-6,7- dihydro-5H-pyrrolo[3,4-b]pyrazin- 5-one |
| 593 | 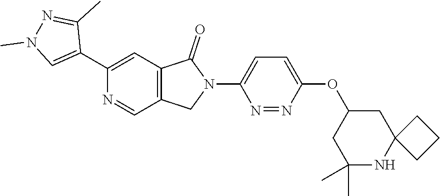 [SMILES: Cc1nn(C)cc1-c1cc2c(cn1)CN(c1ccc(OC3CC(C)(C)NC4(CCC4)C3)nn1)C2=O] | 6-(1,3-dimethyl-1H-pyrazol-4-yl)- 2-(6-((6,6-dimethyl-5- azaspiro[3.5]nonan-8- yl)oxy)pyridazin-3-yl)-2,3- dihydro-1H-pyrrolo[3,4-c]pyridin- 1-one |
| 597 | 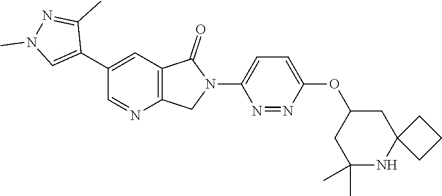 [SMILES: Cc1nn(C)cc1-c1cnc2c(c1)C(=O)N(c1ccc(OC3CC(C)(C)NC4(CCC4)C3)nn1)C2] | 3-(1,3-dimethyl-1H-pyrazol-4-yl)- 6-(6-((6,6-dimethyl-5- azaspiro[3.5]nonan-8- yl)oxy)pyridazin-3-yl)-6,7- dihydro-5H-pyrrolo[3,4-b]pyridin- 5-one |
| 601 | 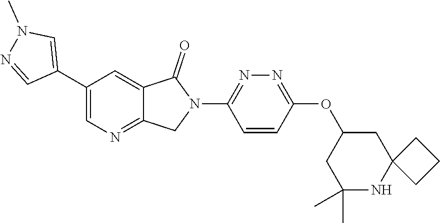 [SMILES: Cn1cc(-c2cnc3c(c2)C(=O)N(c2ccc(OC4CC(C)(C)NC5(CCC5)C4)nn2)C3)cn1] | 6-(6-((6,6-dimethyl-5- azaspiro[3.5]nonan-8- yl)oxy)pyridazin-3-yl)-3-(2- methyl-2H-1,2,3-triazol-4-yl)-6,7- dihydro-5H-pyrrolo[3,4-b]pyridin- 5-one |
| 605 | 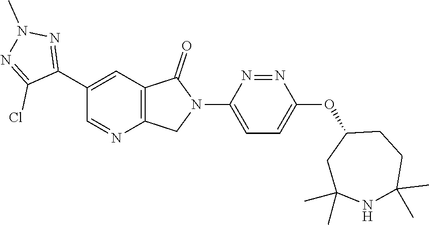 [SMILES: Cn1nc(Cl)c(-c2cnc3c(c2)C(=O)N(c2ccc(O[C@@H]4CCC(C)(C)NC(C)(C)C4)nn2)C3)n1] | (R)-3-(5-chloro-2-methyl-2H- 1,2,3-triazol-4-yl)-6-(6-((2,2,7,7- tetramethylazepan-4- yl)oxy)pyridazin-3-yl)-6,7- dihydro-5H-pyrrolo[3,4-b]pyridin- 5-one |
| 607 | 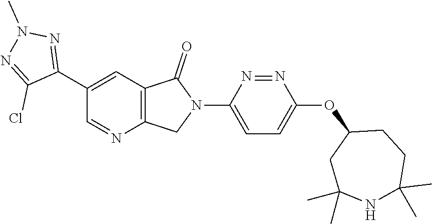 [SMILES: Cn1nc(Cl)c(-c2cnc3c(c2)C(=O)N(c2ccc(O[C@H]4CCC(C)(C)NC(C)(C)C4)nn2)C3)n1] | (S)-3-(5-chloro-2-methyl-2H- 1,2,3-triazol-4-yl)-6-(6-((2,2,7,7- tetramethylazepan-4- yl)oxy)pyridazin-3-yl)-6,7- dihydro-5H-pyrrolo[3,4-b]pyridin- 5-one |
| 608 | 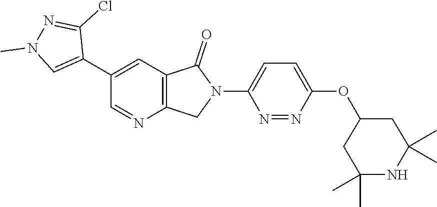 | 3-(3-chloro-1-methyl-1H-pyrazol- 4-yl)-6-(6-((2,2,6,6- tetramethylpiperidin-4- yl)oxy)pyridazin-3-yl)-6,7- dihydro-5H-pyrrolo[3,4-b]pyridin- 5-one |
| 619 | 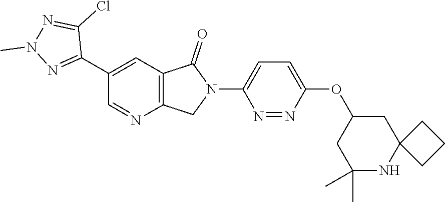 [SMILES: Cn1nc(Cl)c(-c2cnc3c(c2)C(=O)N(c2ccc(OC4CC(C)(C)NC5(CCC5)C4)nn2)C3)n1] | 3-(5-chloro-2-methyl-2H-1,2,3- triazol-4-yl)-6-(6-((6,6-dimethyl-5- azaspiro[3.5]nonan-8- yl)oxy)pyridazin-3-yl)-6,7- dihydro-5H-pyrrolo[3,4-b]pyridin- 5-one |
| 621 | 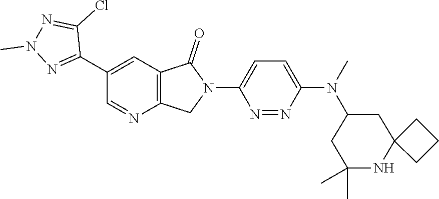 [SMILES: CN(c1ccc(N2Cc3ncc(-c4nn(C)nc4Cl)cc3C2=O)nn1)C1CC(C)(C)NC2(CCC2)C1] | 3-(5-chloro-2-methyl-2H-1,2,3- triazol-4-yl)-6-(6-((6,6-dimethyl-5- azaspiro[3.5]nonan-8- yl)(methyl)amino)pyridazin-3-yl)- 6,7-dihydro-5H-pyrrolo[3,4- b]pyridin-5-one |
| 624 | 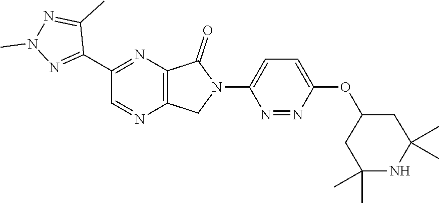 | 3-(2,5-dimethyl-2H-1,2,3-triazol- 4-yl)-6-(6-((2,2,6,6- tetramethylpiperidin-4- yl)oxy)pyridazin-3-yl)-6,7- dihydro-5H-pyrrolo[3,4-b]pyrazin- 5-one |
| 626 | 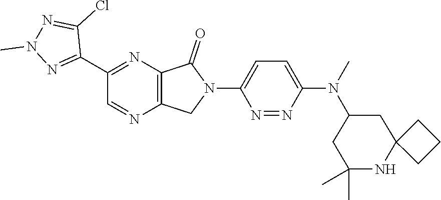 [SMILES: CN(c1ccc(N2Cc3ncc(-c4nn(C)nc4Cl)nc3C2=O)nn1)C1CC(C)(C)NC2(CCC2)C1] | 3-(5-chloro-2-methyl-2H-1,2,3- triazol-4-yl)-6-(6-((6,6-dimethyl-5- azaspiro[3.5]nonan-8- yl)(methyl)amino)pyridazin-3-yl)- 6,7-dihydro-5H-pyrrolo[3,4- b]pyrazin-5-one |
| 628 | 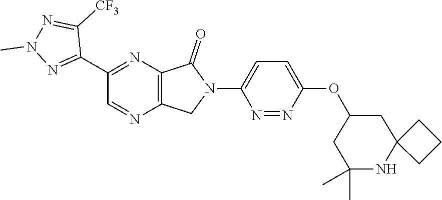 [SMILES: Cn1nc(-c2cnc3c(n2)C(=O)N(c2ccc(OC4CC(C)(C)NC5(CCC5)C4)nn2)C3)c(C(F)(F)F)n1] | 3-(5-chloro-2-methyl-2H-1,2,3- triazol-4-yl)-6-(6-((6,6-dimethyl-5- azaspiro[3.5]nonan-8- yl)oxy)pyridazin-3-yl)-6,7- dihydro-5H-pyrrolo[3,4-b]pyrazin- 5-one |
| 633 | 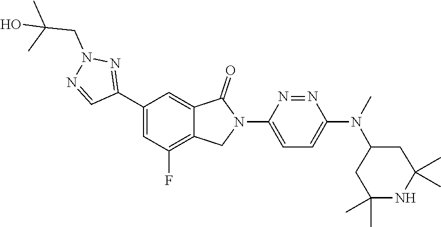 [SMILES: CN(c1ccc(N2Cc3c(F)cc(-c4cnn(CC(C)(C)O)n4)cc3C2=O)nn1)C1CC(C)(C)NC(C)(C)C1] | 4-fluoro-6-(2-(2-hydroxy-2- methylpropyl)-2H-1,2,3-triazol-4- yl)-2-(6-(methyl(2,2,6,6- tetramethylpiperidin-4- yl)amino)pyridazin-3- yl)isoindolin-1-one |
| 635 | 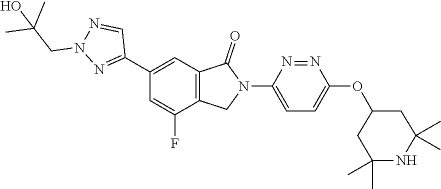 [SMILES: CC(C)(O)Cn1ncc(-c2cc(F)c3c(c2)C(=O)N(c2ccc(OC4CC(C)(C)NC(C)(C)C4)nn2)C3)n1] | 4-fluoro-6-(2-(2-hydroxy-2- methylpropyl)-2H-1,2,3-triazol-4- yl)-2-(6-((2,2,6,6- tetramethylpiperidin-4- yl)oxy)pyridazin-3-yl)isoindolin- 1-one |
| 649 | 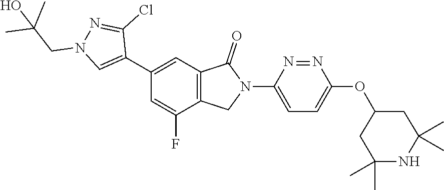 [SMILES: CC(C)(O)Cn1cc(-c2cc(F)c3c(c2)C(=O)N(c2ccc(OC4CC(C)(C)NC(C)(C)C4)nn2)C3)c(Cl)n1] | 6-(3-chloro-1-(2-hydroxy-2- methylpropyl)-1H-pyrazol-4-yl)-4- fluoro-2-(6-((2,2,6,6- tetramethylpiperidin-4- yl)oxy)pyridazin-3-yl)isoindolin- 1-one |
| 664 | 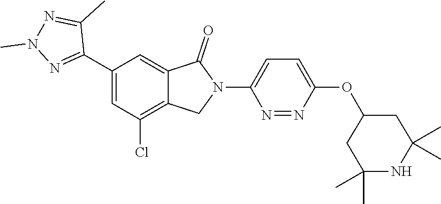 | 4-chloro-6-(2,5-dimethyl-2H- 1,2,3-triazol-4-yl)-2-(6-((2,2,6,6- tetramethylpiperidin-4- yl)oxy)pyridazin-3-yl)isoindolin- 1-one |
| 677 | 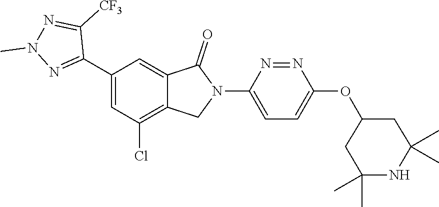 | 4-chloro-6-(5-chloro-2-methyl-2H- 1,2,3-triazol-4-yl)-2-(6-((2,2,6,6- tetramethylpiperidin-4- yl)oxy)pyridazin-3-yl)isoindolin- 1-one |
| 678 | 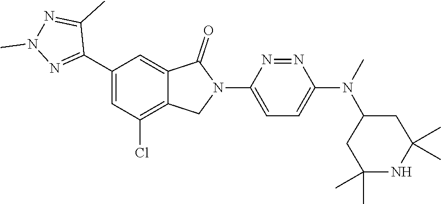 | 6-(2,5-dimethyl-2H-1,2,3-triazol- 4-yl)-2-(6-(methyl(2,2,6,6- tetramethylpiperidin-4- yl)amino)pyridazin-3-yl)-1- oxoisoindoline-4-carbonitrile |
| 680 | 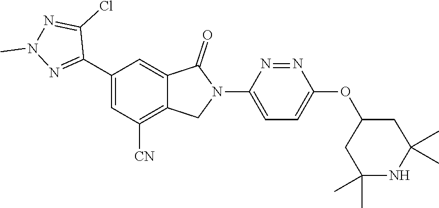 | 6-(5-chloro-2-methyl-2H-1,2,3- triazol-4-yl)-1-oxo-2-(6-((2,2,6,6- tetramethylpiperidin-4- yl)oxy)pyridazin-3-yl)isoindoline- 4-carbonitrile |
| 682 | 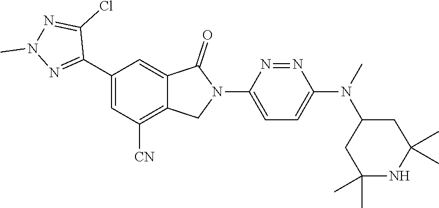 | 6-(5-chloro-2-methyl-2H-1,2,3- triazol-4-yl)-2-(6-(methyl(2,2,6,6- tetramethylpiperidin-4- yl)amino)pyridazin-3-yl)-1- oxoisoindoline-4-carbonitrile |
| 683 | 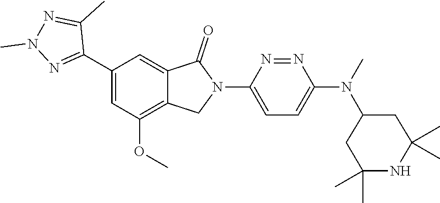 | 6-(2,5-dimethyl-2H-1,2,3-triazol- 4-yl)-4-methoxy-2-(6- (methyl(2,2,6,6- tetramethylpiperidin-4- yl)amino)pyridazin-3- yl)isoindolin-1-one |
| 688 | 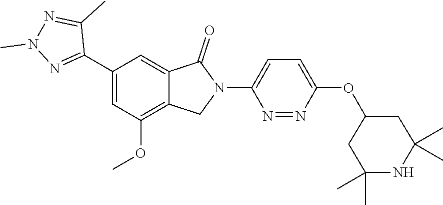 | 6-(2,5-dimethyl-2H-1,2,3-triazol- 4-yl)-4-methoxy-2-(6-((2,2,6,6- tetramethylpiperidin-4- yl)oxy)pyridazin-3-yl)isoindolin- 1-one |
| 696 | 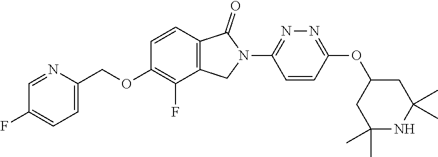 [SMILES: CC1(C)CC(Oc2ccc(N3Cc4c(ccc(OCc5ccc(F)cn5)c4F)C3=O)nn2)CC(C)(C)N1] | 4-fluoro-5-((5-fluoropyridin-2- yl)methoxy)-2-(6-((2,2,6,6- tetramethylpiperidin-4- yl)oxy)pyridazin-3-yl)isoindolin- 1-one |

TABLE 3B

| No. | Chemical Structure | Name |
|---|---|---|
| 19 | 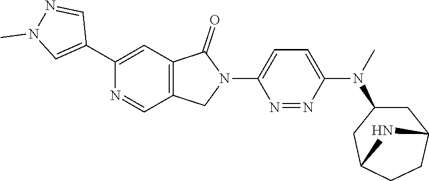 [SMILES: CN(c1ccc(N2Cc3cnc(-c4cnn(C)c4)cc3C2=O)nn1)[C@@H]1C[C@H]2CC[C@@H](C1)N2] | 2-(6-(((1R,3s,5S)-8- azabicyclo[3.2.1]octan-3- yl)(methyl)amino)pyridazin-3-yl)- 6-(1-methyl-1H-pyrazol-4-yl)-2,3- dihydro-1H-pyrrolo[3,4-c]pyridin- 1-one |
| 20 | 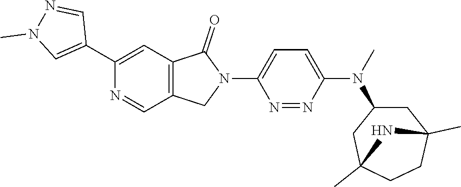 | 2-(6-(((1R,3s,5S)-1,5-dimethyl-8- azabicyclo[3.2.1]octan-3- yl)(methyl)amino)pyridazin-3-yl)- 6-(1-methyl-1H-pyrazol-4-yl)-2,3- dihydro-1H-pyrrolo[3,4-c]pyridin- 1-one |
| 27 | 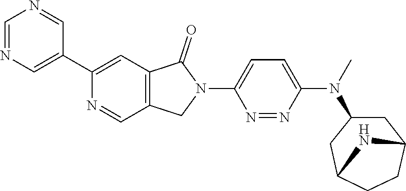 [SMILES: CN(c1ccc(N2Cc3cnc(-c4cncnc4)cc3C2=O)nn1)[C@@H]1C[C@H]2CC[C@@H](C1)N2] | 2-(6-(((1R,3s,5S)-8- azabicyclo[3.2.1]octan-3- yl)(methyl)amino)pyridazin-3-yl)- 6-(pyrimidin-5-yl)-2,3-dihydro- 1H-pyrrolo[3,4-c]pyridin-1-one |
| 42 | 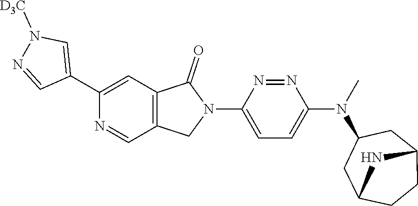 [SMILES: [2H]C([2H])([2H])n1cc(-c2cc3c(cn2)CN(c2ccc(N(C)[C@@H]4C[C@H]5CC[C@@H](C4)N5)nn2)C3=O)cn1] | 2-(6-(((1R,3s,5S)-8- azabicyclo[3.2.1]octan-3- yl)(methyl)amino)pyridazin-3-yl)- 6-(1-(methyl-d3)-1H-pyrazol-4- yl)-2,3-dihydro-1H-pyrrolo[3,4- c]pyridin-1-one |
| 55 | 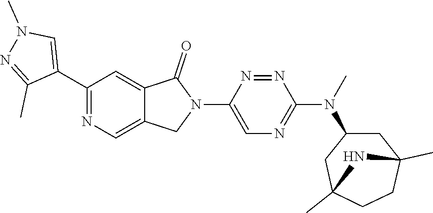 [SMILES: Cc1nn(C)cc1-c1cc2c(cn1)CN(c1cnc(N(C)[C@H]3C[C@]4(C)CC[C@](C)(C3)N4)nn1)C2=O] | 6-(1,3-dimethyl-1H-pyrazol-4-yl)- 2-(3-(((1R,3s,5S)-1,5-dimethyl-8- azabicyclo[3.2.1]octan-3- yl)(methyl)amino)-1,2,4-triazin-6- yl)-2,3-dihydro-1H-pyrrolo[3,4- c]pyridin-1-one |
| 60 | 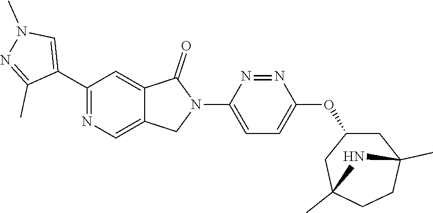 [SMILES: Cc1nn(C)cc1-c1cc2c(cn1)CN(c1ccc(O[C@@H]3C[C@]4(C)CC[C@](C)(C3)N4)nn1)C2=O] | 6-(1,3-dimethyl-1H-pyrazol-4-yl)- 2-(6-(((1R,3r,5S)-1,5-dimethyl-8- azabicyclo[3.2.1]octan-3- yl)oxy)pyridazin-3-yl)-2,3- dihydro-1H-pyrrolo[3,4-c]pyridin- 1-one |
| 61 | 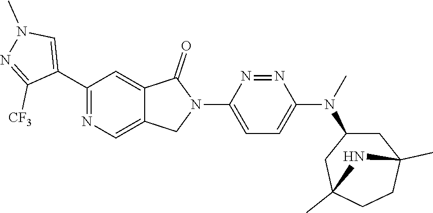 [SMILES: CN(c1ccc(N2Cc3cnc(-c4cn(C)nc4C(F)(F)F)cc3C2=O)nn1)[C@H]1C[C@]2(C)CC[C@](C)(C1)N2] | 2-(6-(((1R,3s,5S)-1,5-dimethyl-8- azabicyclo[3.2.1]octan-3- yl)(methyl)amino)pyridazin-3-yl)- 6-(1-methyl-3-(trifluoromethyl)- 1H-pyrazol-4-yl)-2,3-dihydro-1H- pyrrolo[3,4-c]pyridin-1-one |
| 62 | 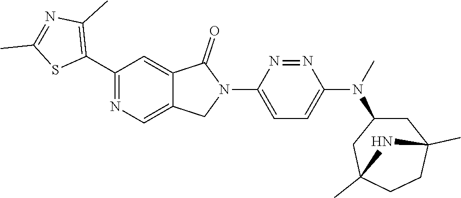 | 2-(6-(((1R,3s,5S)-1,5-dimethyl-8- azabicyclo[3.2.1]octan-3- yl)(methyl)amino)pyridazin-3-yl)- 6-(2,4-dimethylthiazol-5-yl)-2,3- dihydro-1H-pyrrolo[3,4-c]pyridin- 1-one |
| 67 | 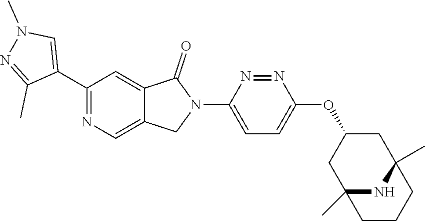 [SMILES: Cc1nn(C)cc1-c1cc2c(cn1)CN(c1ccc(O[C@@H]3C[C@]4(C)CCC[C@](C)(C3)N4)nn1)C2=O] | 6-(1,3-dimethyl-1H-pyrazol-4-yl)- 2-(6-(((1R,3r,5S)-1,5-dimethyl-9- azabicyclo[3.3.1]nonan-3- yl)oxy)pyridazin-3-yl)-2,3- dihydro-1H-pyrrolo[3,4-c]pyridin- 1-one |
| 68 | 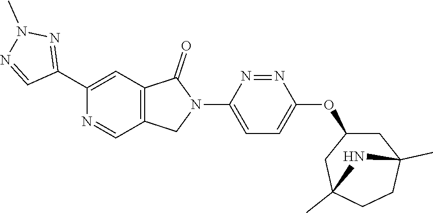 [SMILES: Cn1ncc(-c2cc3c(cn2)CN(c2ccc(O[C@H]4C[C@]5(C)CC[C@](C)(C4)N5)nn2)C3=O)n1] | 2-(6-(((1R,3s,5S)-1,5-dimethyl-8- azabicyclo[3.2.1]octan-3- yl)oxy)pyridazin-3-yl)-6-(2- methyl-2H-1,2,3-triazol-4-yl)-2,3- dihydro-1H-pyrrolo[3,4-c]pyridin- 1-one |
| 69 | 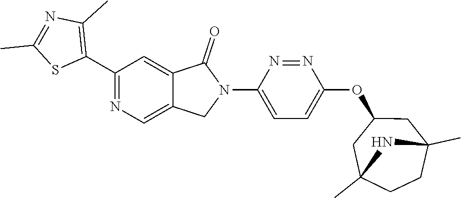 | 2-(6-(((1R,3s,5S)-1,5-dimethyl-8- azabicyclo[3.2.1]octan-3- yl)oxy)pyridazin-3-yl)-6-(2,4- dimethylthiazol-5-yl)-2,3-dihydro- 1H-pyrrolo[3,4-c]pyridin-1-one |
| 70 | 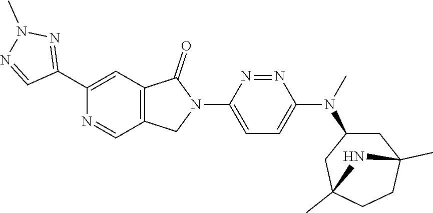 [SMILES: CN(c1ccc(N2Cc3cnc(-c4cnn(C)n4)cc3C2=O)nn1)[C@H]1C[C@]2(C)CC[C@](C)(C1)N2] | 2-(6-(((1R,3s,5S)-1,5-dimethyl-8- azabicyclo[3.2.1]octan-3- yl)(methyl)amino)pyridazin-3-yl)- 6-(2-methyl-2H-1,2,3-triazol-4- yl)-2,3-dihydro-1H-pyrrolo[3,4- c]pyridin-1-one |
| 72 | 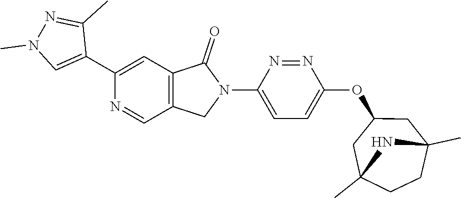 | 6-(1,3-dimethyl-1H-pyrazol-4-yl)- 2-(6-(((1R,3s,5S)-1,5-dimethyl-8- azabicyclo[3.2.1]octan-3- yl)oxy)pyridazin-3-yl)-2,3- dihydro-1H-pyrrolo[3,4-c]pyridin- 1-one |
| 73 | 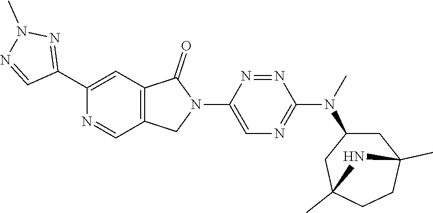 [SMILES: CN(c1ncc(N2Cc3cnc(-c4cnn(C)n4)cc3C2=O)nn1)[C@H]1C[C@]2(C)CC[C@](C)(C1)N2] | 2-(3-(((1R,3s,5S)-1,5-dimethyl-8- azabicyclo[3.2.1]octan-3- yl)(methyl)amino)-1,2,4-triazin-6- yl)-6-(2-methyl-2H-1,2,3-triazol- 4-yl)-2,3-dihydro-1H-pyrrolo[3,4- c]pyridin-1-one |
| 74 | 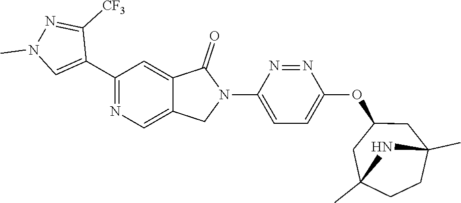 | 2-(6-(((1R,3s,5S)-1,5-dimethyl-8- azabicyclo[3.2.1]octan-3- yl)oxy)pyridazin-3-yl)-6-(1- methyl-3-(trifluoromethyl)-1H- pyrazol-4-yl)-2,3-dihydro-1H- pyrrolo[3,4-c]pyridin-1-one |
| 77 | 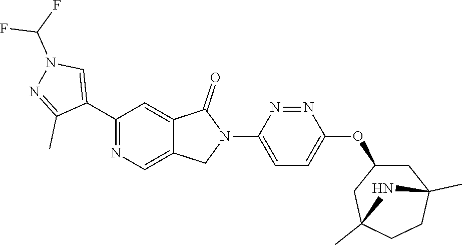 [SMILES: Cc1nn(C(F)F)cc1-c1cc2c(cn1)CN(c1ccc(O[C@H]3C[C@]4(C)CC[C@](C)(C3)N4)nn1)C2=O] | 6-(1-(difluoromethyl)-3-methyl- 1H-pyrazol-4-yl)-2-(6- (((1R,3s,5S)-1,5-dimethyl-8- azabicyclo[3.2.1]octan-3- yl)oxy)pyridazin-3-yl)-2,3- dihydro-1H-pyrrolo[3,4-c]pyridin- 1-one |
| 79 | 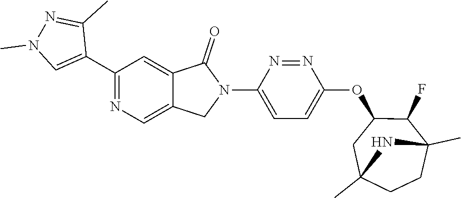 | 6-(1,3-dimethyl-1H-pyrazol-4-yl)- 2-(6-(((1S,2S,3R,5R)-2-fluoro-1,5- dimethyl-8- azabicyclo[3.2.1]octan-3- yl)oxy)pyridazin-3-yl)-2,3- dihydro-1H-pyrrolo[3,4-c]pyridin- 1-one |
| 82 | 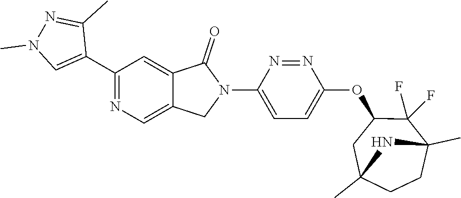 | 2-(6-(((1R,2S,3R,4S,5S)-2,4- difluoro-1,5-dimethyl-8- azabicyclo[3.2.1]octan-3- yl)oxy)pyridazin-3-yl)-6-(1,3- dimethyl-1H-pyrazol-4-yl)-2,3- dihydro-1H-pyrrolo[3,4-c]pyridin- 1-one |
| 85 | 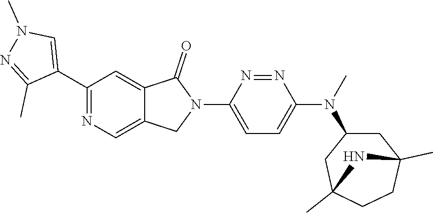 [SMILES: Cc1nn(C)cc1-c1cc2c(cn1)CN(c1ccc(N(C)[C@H]3C[C@]4(C)CC[C@](C)(C3)N4)nn1)C2=O] | 6-(1,3-dimethyl-1H-pyrazol-4-yl)- 2-(6-(((1R,3s,5S)-1,5-dimethyl-8- azabicyclo[3.2.1]octan-3- yl)(methyl)amino)pyridazin-3-yl)- 2,3-dihydro-1H-pyrrolo[3,4- c]pyridin-1-one |
| 89 | 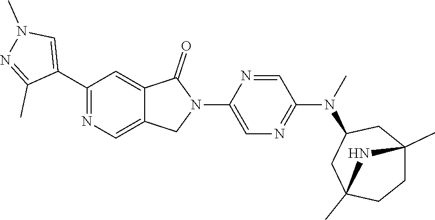 [SMILES: Cc1nn(C)cc1-c1cc2c(cn1)CN(c1cnc(N(C)[C@H]3C[C@]4(C)CC[C@](C)(C3)N4)cn1)C2=O] | 6-(1,3-dimethyl-1H-pyrazol-4-yl)- 2-(5-(((1R,3s,5S)-1,5-dimethyl-8- azabicyclo[3.2.1]octan-3- yl)(methyl)amino)pyrazin-2-yl)- 2,3-dihydro-1H-pyrrolo[3,4- c]pyridin-1-one |
| 90 | 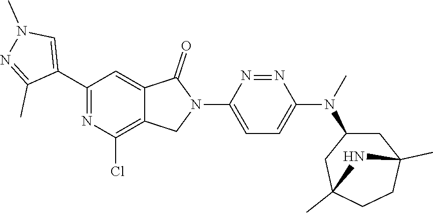 [SMILES: Cc1nn(C)cc1-c1cc2c(c(Cl)n1)CN(c1ccc(N(C)[C@H]3C[C@]4(C)CC[C@](C)(C3)N4)nn1)C2=O] | 4-chloro-6-(1,3-dimethyl-1H- pyrazol-4-yl)-2-(6-(((1R,3s,5S)- 1,5-dimethyl-8- azabicyclo[3.2.1]octan-3- yl)(methyl)amino)pyridazin-3-yl)- 2,3-dihydro-1H-pyrrolo[3,4- c]pyridin-1-one |
| 93 | 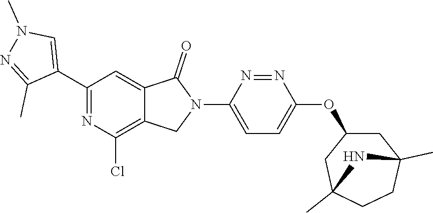 [SMILES: Cc1nn(C)cc1-c1cc2c(c(Cl)n1)CN(c1ccc(O[C@H]3C[C@]4(C)CC[C@](C)(C3)N4)nn1)C2=O] | 4-chloro-6-(1,3-dimethyl-1H- pyrazol-4-yl)-2-(6-(((1R,3s,5S)- 1,5-dimethyl-8- azabicyclo[3.2.1]octan-3- yl)oxy)pyridazin-3-yl)-2,3- dihydro-1H-pyrrolo[3,4-c]pyridin- 1-one |
| 94 | 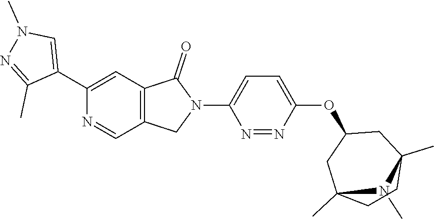 [SMILES: Cc1nn(C)cc1-c1cc2c(cn1)CN(c1ccc(O[C@H]3C[C@]4(C)CC[C@](C)(C3)N4C)nn1)C2=O] | 6-(1,3-dimethyl-1H-pyrazol-4-yl)- 2-(6-(((1R,3s,5S)-1,5,8-trimethyl- 8-azabicyclo[3.2.1]octan-3- yl)oxy)pyridazin-3-yl)-2,3- dihydro-1H-pyrrolo[3,4-c]pyridin- 1-one |
| 95 | 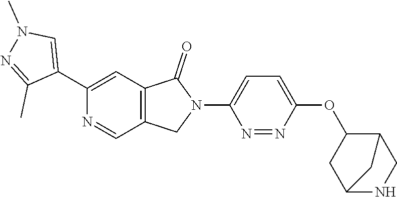 [SMILES: Cc1nn(C)cc1-c1cc2c(cn1)CN(c1ccc(OC3CC4CC3CN4)nn1)C2=O] | 2-(6-((2-azabicyclo[2.2.1]heptan- 5-yl)oxy)pyridazin-3-yl)-6-(1,3- dimethyl-1H-pyrazol-4-yl)-2,3- dihydro-1H-pyrrolo[3,4-c]pyridin- 1-one |
| 96 | 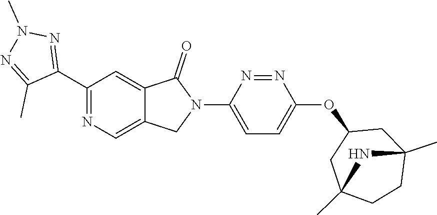 [SMILES: Cc1nn(C)nc1-c1cc2c(cn1)CN(c1ccc(O[C@H]3C[C@]4(C)CC[C@](C)(C3)N4)nn1)C2=O] | 6-(2,5-dimethyl-2H-1,2,3-triazol- 4-yl)-2-(6-(((1R,3s,5S)-1,5- dimethyl-8- azabicyclo[3.2.1]octan-3- yl)oxy)pyridazin-3-yl)-2,3- dihydro-1H-pyrrolo[3,4-c]pyridin- 1-one |
| 97 | 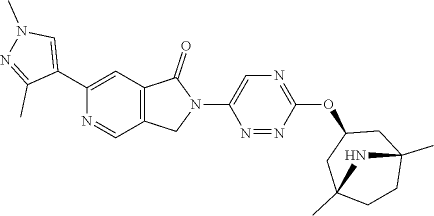 [SMILES: Cc1nn(C)cc1-c1cc2c(cn1)CN(c1cnc(O[C@H]3C[C@]4(C)CC[C@](C)(C3)N4)nn1)C2=O] | 6-(1,3-dimethyl-1H-pyrazol-4-yl)- 2-(3-(((1R,3s,5S)-1,5-dimethyl-8- azabicyclo[3.2.1]octan-3-yl)oxy)- 1,2,4-triazin-6-yl)-2,3-dihydro-1H- pyrrolo[3,4-c]pyridin-1-one |
| 98 | 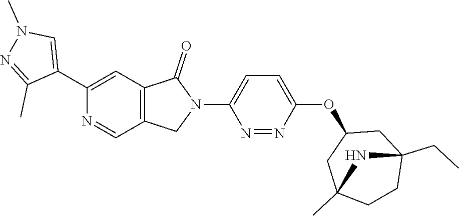 [SMILES: CC[C@]12CC[C@](C)(C[C@@H](Oc3ccc(N4Cc5cnc(-c6cn(C)nc6C)cc5C4=O)nn3)C1)N2] | 6-(1,3-dimethyl-1H-pyrazol-4-yl)- 2-(6-(((1S,3R,5R)-1-ethyl-5- methyl-8-azabicyclo[3.2.1]octan- 3-yl)oxy)pyridazin-3-yl)-2,3- dihydro-1H-pyrrolo[3,4-c]pyridin- 1-one |
| 99 | 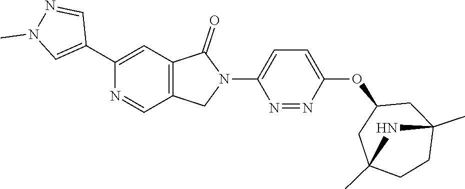 | 2-(6-(((1R,3s,5S)-1,5-dimethyl-8- azabicyclo[3.2.1]octan-3- yl)oxy)pyridazin-3-yl)-6-(1- methyl-1H-pyrazol-4-yl)-2,3- dihydro-1H-pyrrolo[3,4-c]pyridin- 1-one |
| 100 | 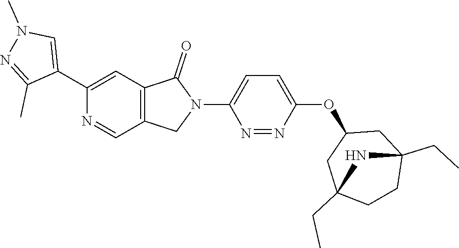 [SMILES: CC[C@]12CC[C@](CC)(C[C@@H](Oc3ccc(N4Cc5cnc(-c6cn(C)nc6C)cc5C4=O)nn3)C1)N2] | 2-(6-(((1R,3s,5S)-1,5-diethyl-8- azabicyclo[3.2.1]octan-3- yl)oxy)pyridazin-3-yl)-6-(1,3- dimethyl-1H-pyrazol-4-yl)-2,3- dihydro-1H-pyrrolo[3,4-c]pyridin- 1-one |
| 102 | 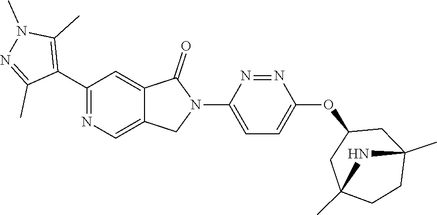 [SMILES: Cc1nn(C)c(C)c1-c1cc2c(cn1)CN(c1ccc(O[C@H]3C[C@]4(C)CC[C@](C)(C3)N4)nn1)C2=O] | 2-(6-(((1R,3s,5S)-1,5-dimethyl-8- azabicyclo[3.2.1]octan-3- yl)oxy)pyridazin-3-yl)-6-(1,3,5- trimethyl-1H-pyrazol-4-yl)-2,3- dihydro-1H-pyrrolo[3,4-c]pyridin- 1-one |
| 105 | 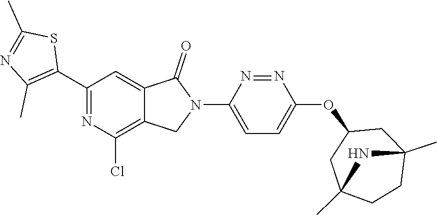 [SMILES: Cc1nc(C)c(-c2cc3c(c(Cl)n2)CN(c2ccc(O[C@H]4C[C@]5(C)CC[C@](C)(C4)N5)nn2)C3=O)s1] | 4-chloro-2-(6-(((1R,3s,5S)-1,5- dimethyl-8- azabicyclo[3.2.1]octan-3- yl)oxy)pyridazin-3-yl)-6-(2,4- dimethylthiazol-5-yl)-2,3-dihydro- 1H-pyrrolo[3,4-c]pyridin-1-one |
| 106 | 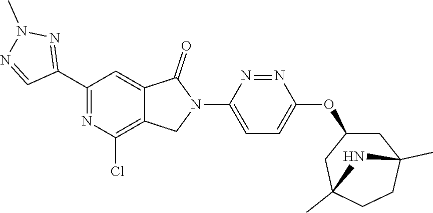 [SMILES: Cn1ncc(-c2cc3c(c(Cl)n2)CN(c2ccc(O[C@H]4C[C@]5(C)CC[C@](C)(C4)N5)nn2)C3=O)n1] | 4-chloro-2-(6-(((1R,3s,5S)-1,5- dimethyl-8- azabicyclo[3.2.1]octan-3- yl)oxy)pyridazin-3-yl)-6-(2- methyl-2H-1,2,3-triazol-4-yl)-2,3- dihydro-1H-pyrrolo[3,4-c]pyridin- 1-one |
| 107 | 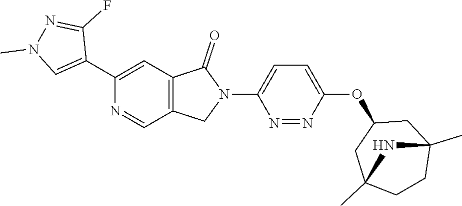 | 2-(6-(((1R,3s,5S)-1,5-dimethyl-8- azabicyclo[3.2.1]octan-3- yl)oxy)pyridazin-3-yl)-6-(3-fluoro- 1-methyl-1H-pyrazol-4-yl)-2,3- dihydro-1H-pyrrolo[3,4-c]pyridin- 1-one |
| 108 | 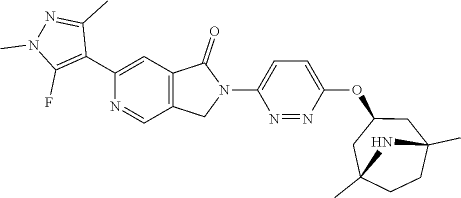 | 2-(6-(((1R,3s,5S)-1,5-dimethyl-8- azabicyclo[3.2.1]octan-3- yl)oxy)pyridazin-3-yl)-6-(5-fluoro- 1,3-dimethyl-1H-pyrazol-4-yl)- 2,3-dihydro-1H-pyrrolo[3,4- c]pyridin-1-one |
| 110 | 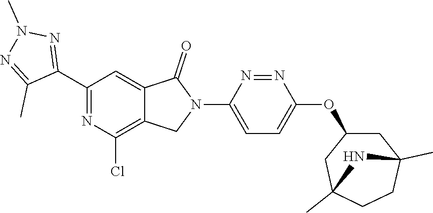 [SMILES: Cc1nn(C)nc1-c1cc2c(c(Cl)n1)CN(c1ccc(O[C@H]3C[C@]4(C)CC[C@](C)(C3)N4)nn1)C2=O] | 4-chloro-6-(2,5-dimethyl-2H- 1,2,3-triazol-4-yl)-2-(6- (((1R,3s,5S)-1,5-dimethyl-8- azabicyclo[3.2.1]octan-3- yl)oxy)pyridazin-3-yl)-2,3- dihydro-1H-pyrrolo[3,4-c]pyridin- 1-one |
| 111 | 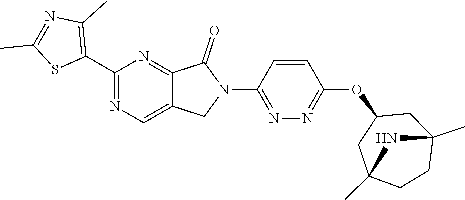 | 6-(6-(((1R,3s,5S)-1,5-dimethyl-8- azabicyclo[3.2.1]octan-3- yl)oxy)pyridazin-3-yl)-2-(2,4- dimethylthiazol-5-yl)-5,6-dihydro- 7H-pyrrolo[3,4-d]pyrimidin-7-one |
| 112 | 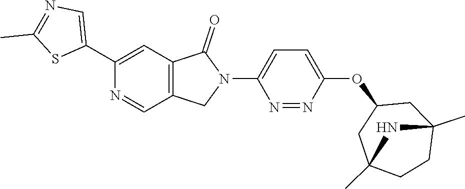 | 2-(6-(((1R,3s,5S)-1,5-dimethyl-8- azabicyclo[3.2.1]octan-3- yl)oxy)pyridazin-3-yl)-6-(2- methylthiazol-5-yl)-2,3-dihydro- 1H-pyrrolo[3,4-c]pyridin-1-one |
| 114 | 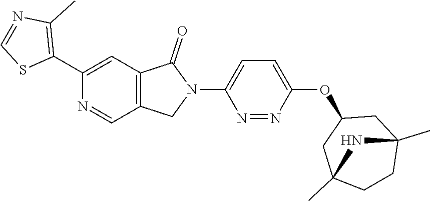 [SMILES: Cc1ncsc1-c1cc2c(cn1)CN(c1ccc(O[C@H]3C[C@]4(C)CC[C@](C)(C3)N4)nn1)C2=O] | 2-(6-(((1R,3s,5S)-1,5-dimethyl-8- azabicyclo[3.2.1]octan-3- yl)oxy)pyridazin-3-yl)-6-(4- methylthiazol-5-yl)-2,3-dihydro- 1H-pyrrolo[3,4-c]pyridin-1-one |
| 116 | 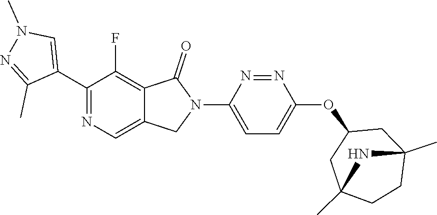 [SMILES: Cc1nn(C)cc1-c1ncc2c(c1F)C(=O)N(c1ccc(O[C@H]3C[C@]4(C)CC[C@](C)(C3)N4)nn1)C2] | 6-(1,3-dimethyl-1H-pyrazol-4-yl)- 2-(6-(((1R,3s,5S)-1,5-dimethyl-8- azabicyclo[3.2.1]octan-3- yl)oxy)pyridazin-3-yl)-7-fluoro- 2,3-dihydro-1H-pyrrolo[3,4- c]pyridin-1-one |
| 117 | 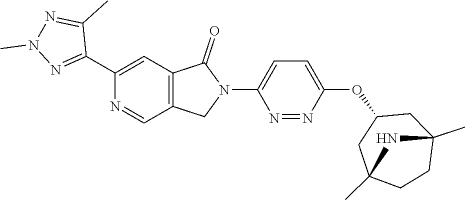 | 6-(2,5-dimethyl-2H-1,2,3-triazol- 4-yl)-2-(6-(((1R,3r,5S)-1,5- dimethyl-8- azabicyclo[3.2.1]octan-3- yl)oxy)pyridazin-3-yl)-2,3- dihydro-1H-pyrrolo[3,4-c]pyridin- 1-one |
| 118 | 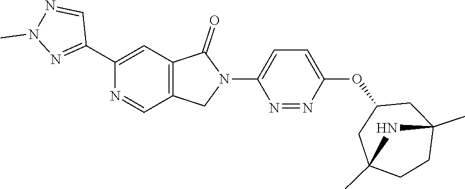 | 2-(6-(((1R,3r,5S)-1,5-dimethyl-8- azabicyclo[3.2.1]octan-3- yl)oxy)pyridazin-3-yl)-6-(2- methyl-2H-1,2,3-triazol-4-yl)-2,3- dihydro-1H-pyrrolo[3,4-c]pyridin- 1-one |
| 164 | 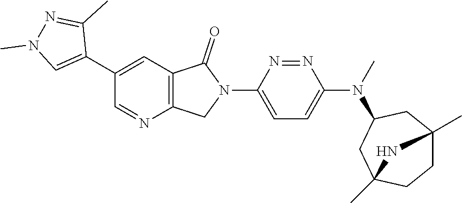 | 3-(1,3-dimethyl-1H-pyrazol-4-yl)- 6-(6-(((1R,3s,5S)-1,5-dimethyl-8- azabicyclo[3.2.1]octan-3- yl)(methyl)amino)pyridazin-3-yl)- 6,7-dihydro-5H-pyrrolo[3,4- b]pyridin-5-one |
| 166 | 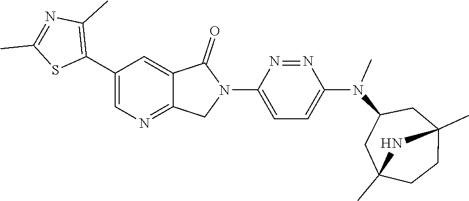 | 6-(6-(((1R,3s,5S)-1,5-dimethyl-8- azabicyclo[3.2.1]octan-3- yl)(methyl)amino)pyridazin-3-yl)- 3-(2,4-dimethylthiazol-5-yl)-6,7- dihydro-5H-pyrrolo[3,4-b]pyridin- 5-one |
| 169 | 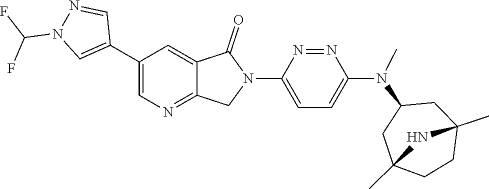 [SMILES: CN(c1ccc(N2Cc3ncc(-c4cnn(C(F)F)c4)cc3C2=O)nn1)[C@H]1C[C@]2(C)CC[C@](C)(C1)N2] | 3-(1-(difluoromethyl)-1H-pyrazol- 4-yl)-6-(6-(((1R,3s,5S)-1,5- dimethyl-8- azabicyclo[3.2.1]octan-3- yl)(methyl)amino)pyridazin-3-yl)- 6,7-dihydro-5H-pyrrolo[3,4- b]pyridin-5-one |
| 171 | 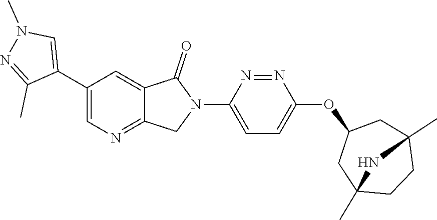 [SMILES: Cc1nn(C)cc1-c1cnc2c(c1)C(=O)N(c1ccc(O[C@H]3C[C@]4(C)CC[C@](C)(C3)N4)nn1)C2] | 3-(1,3-dimethyl-1H-pyrazol-4-yl)- 6-(6-(((1R,3s,5S)-1,5-dimethyl-8- azabicyclo[3.2.1]octan-3- yl)oxy)pyridazin-3-yl)-6,7- dihydro-5H-pyrrolo[3,4-b]pyridin- 5-one |
| 174 | 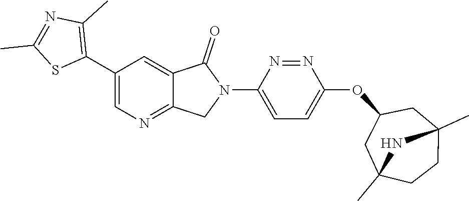 | 6-(6-(((1R,3s,5S)-1,5-dimethyl-8- azabicyclo[3.2.1]octan-3- yl)oxy)pyridazin-3-yl)-3-(2,4- dimethylthiazol-5-yl)-6,7-dihydro- 5H-pyrrolo[3,4-b]pyridin-5-one |
| 175 | 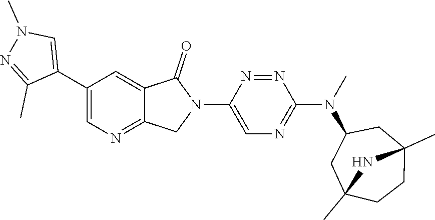 [SMILES: Cc1nn(C)cc1-c1cnc2c(c1)C(=O)N(c1cnc(N(C)[C@H]3C[C@]4(C)CC[C@](C)(C3)N4)nn1)C2] | 3-(1,3-dimethyl-1H-pyrazol-4-yl)- 6-(3-(((1R,3s,5S)-1,5-dimethyl-8- azabicyclo[3.2.1]octan-3- yl)(methyl)amino)-1,2,4-triazin-6- yl)-6,7-dihydro-5H-pyrrolo[3,4- b]pyridin-5-one |
| 178 | 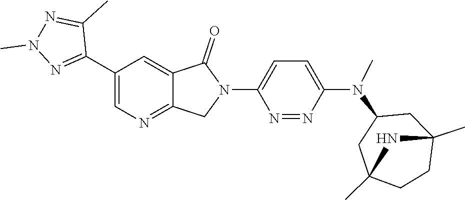 | 3-(2,5-dimethyl-2H-1,2,3-triazol- 4-yl)-6-(6-(((1R,3s,5S)-1,5- dimethyl-8- azabicyclo[3.2.1]octan-3- yl)(methyl)amino)pyridazin-3-yl)- 6,7-dihydro-5H-pyrrolo[3,4- b]pyridin-5-one |
| 181 | 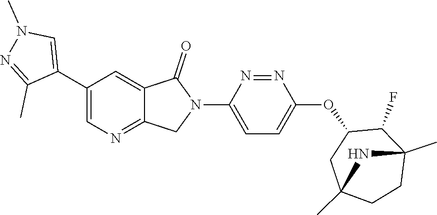 [SMILES: Cc1nn(C)cc1-c1cnc2c(c1)C(=O)N(c1ccc(O[C@H]3C[C@@]4(C)CC[C@](C)(N4)[C@H]3F)nn1)C2] | 3-(1,3-dimethyl-1H-pyrazol-4-yl)- 6-(6-(((1S,2R,3S,5R)-2-fluoro-1,5- dimethyl-8- azabicyclo[3.2.1]octan-3- yl)oxy)pyridazin-3-yl)-6,7- dihydro-5H-pyrrolo[3,4-b]pyridin- 5-one |
| 183 | 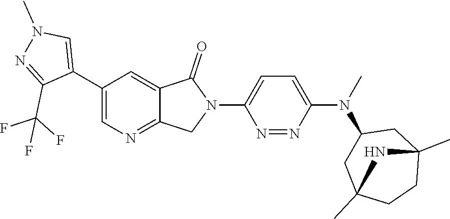 [SMILES: CN(c1ccc(N2Cc3ncc(-c4cn(C)nc4C(F)(F)F)cc3C2=O)nn1)[C@H]1C[C@]2(C)CC[C@](C)(C1)N2] | 6-(6-(((1R,3s,5S)-1,5-dimethyl-8- azabicyclo[3.2.1]octan-3- yl)(methyl)amino)pyridazin-3-yl)- 3-(1-methyl-3-(trifluoromethyl)- 1H-pyrazol-4-yl)-6,7-dihydro-5H- pyrrolo[3,4-b]pyridin-5-one |
| 185 | 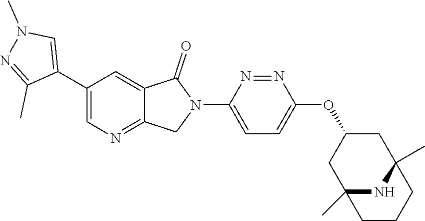 [SMILES: Cc1nn(C)cc1-c1cnc2c(c1)C(=O)N(c1ccc(O[C@@H]3C[C@]4(C)CCC[C@](C)(C3)N4)nn1)C2] | 3-(1,3-dimethyl-1H-pyrazol-4-yl)- 6-(6-(((1R,3r,5S)-1,5-dimethyl-9- azabicyclo[3.3.1]nonan-3- yl)oxy)pyridazin-3-yl)-6,7- dihydro-5H-pyrrolo[3,4-b]pyridin- 5-one |
| 186 | 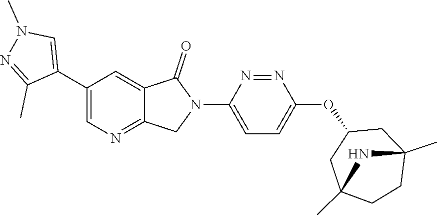 [SMILES: Cc1nn(C)cc1-c1cnc2c(c1)C(=O)N(c1ccc(O[C@@H]3C[C@]4(C)CC[C@](C)(C3)N4)nn1)C2] | 3-(1,3-dimethyl-1H-pyrazol-4-yl)- 6-(6-(((1R,3r,5S)-1,5-dimethyl-8- azabicyclo[3.2.1]octan-3- yl)oxy)pyridazin-3-yl)-6,7- dihydro-5H-pyrrolo[3,4-b]pyridin- 5-one |
| 188 | 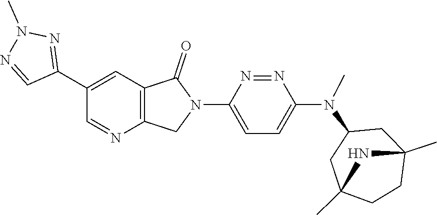 [SMILES: CN(c1ccc(N2Cc3ncc(-c4cnn(C)n4)cc3C2=O)nn1)[C@H]1C[C@]2(C)CC[C@](C)(C1)N2] | 6-(6-(((1R,3s,5S)-1,5-dimethyl-8- azabicyclo[3.2.1]octan-3- yl)(methyl)amino)pyridazin-3-yl)- 3-(2-methyl-2H-1,2,3-triazol-4- yl)-6,7-dihydro-5H-pyrrolo[3,4- b]pyridin-5-one |
| 194 | 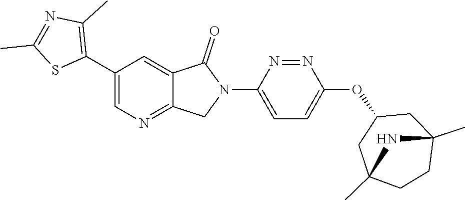 | 6-(6-(((1R,3r,5S)-1,5-dimethyl-8- azabicyclo[3.2.1]octan-3- yl)oxy)pyridazin-3-yl)-3-(2,4- dimethylthiazol-5-yl)-6,7-dihydro- 5H-pyrrolo[3,4-b]pyridin-5-one |
| 196 | 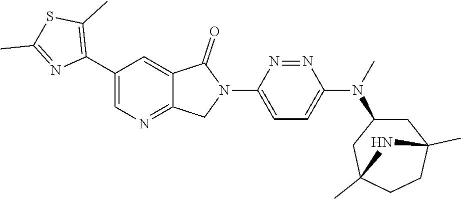 | 6-(6-(((1R,3s,5S)-1,5-dimethyl-8- azabicyclo[3.2.1]octan-3- yl)(methyl)amino)pyridazin-3-yl)- 3-(2,5-dimethylthiazol-4-yl)-6,7- dihydro-5H-pyrrolo[3,4-b]pyridin- 5-one |
| 197 | 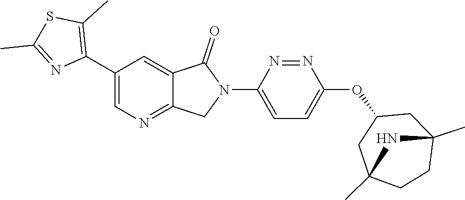 | 6-(6-(((1R,3r,5S)-1,5-dimethyl-8- azabicyclo[3.2.1]octan-3- yl)oxy)pyridazin-3-yl)-3-(2,5- dimethylthiazol-4-yl)-6,7-dihydro- 5H-pyrrolo[3,4-b]pyridin-5-one |
| 199 | 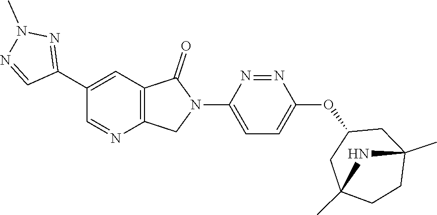 [SMILES: Cn1ncc(-c2cnc3c(c2)C(=O)N(c2ccc(O[C@@H]4C[C@]5(C)CC[C@](C)(C4)N5)nn2)C3)n1] | 6-(6-(((1R,3s,5S)-1,5-dimethyl-8- azabicyclo[3.2.1]octan-3- yl)oxy)pyridazin-3-yl)-3-(2- methyl-2H-1,2,3-triazol-4-yl)-6,7- dihydro-5H-pyrrolo[3,4-b]pyridin- 5-one |
| 200 | 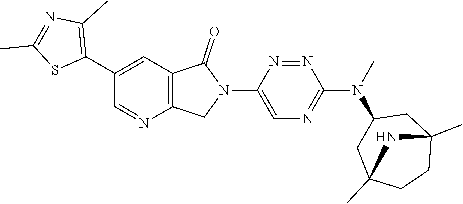 | 6-(3-(((1R,3s,5S)-1,5-dimethyl-8- azabicyclo[3.2.1]octan-3- yl)(methyl)amino)-1,2,4-triazin-6- yl)-3-(2,4-dimethylthiazol-5-yl)- 6,7-dihydro-5H-pyrrolo[3,4- b]pyridin-5-one |
| 201 | 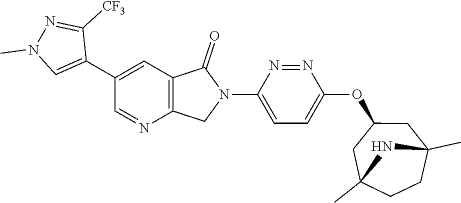 | 6-(6-(((1R,3s,5S)-1,5-dimethyl-8- azabicyclo[3.2.1]octan-3- yl)oxy)pyridazin-3-yl)-3-(1- methyl-3-(trifluoromethyl)-1H- pyrazol-4-yl)-6,7-dihydro-5H- pyrrolo[3,4-b]pyridin-5-one |
| 202 | 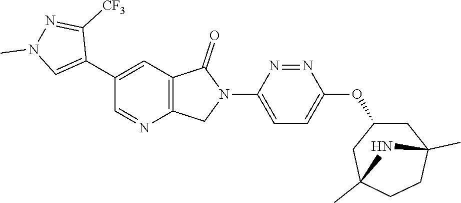 | 6-(6-(((1R,3r,5S)-1,5-dimethyl-8- azabicyclo[3.2.1]octan-3- yl)oxy)pyridazin-3-yl)-3-(1- methyl-3-(trifluoromethyl)-1H- pyrazol-4-yl)-6,7-dihydro-5H- pyrrolo[3,4-b]pyridin-5-one |
| 206 | 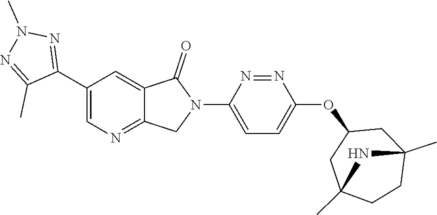 [SMILES: Cc1nn(C)nc1-c1cnc2c(c1)C(=O)N(c1ccc(O[C@H]3C[C@]4(C)CC[C@](C)(C3)N4)nn1)C2] | 3-(2,5-dimethyl-2H-1,2,3-triazol- 4-yl)-6-(6-(((1R,3s,5S)-1,5- dimethyl-8- azabicyclo[3.2.1]octan-3- yl)oxy)pyridazin-3-yl)-6,7- dihydro-5H-pyrrolo[3,4-b]pyridin- 5-one |
| 209 | 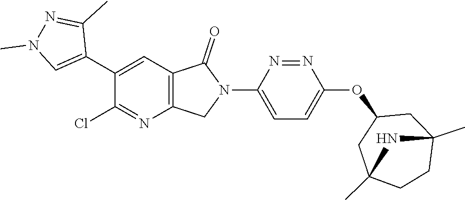 | 2-chloro-3-(1,3-dimethyl-1H- pyrazol-4-yl)-6-(6-(((1R,3s,5S)- 1,5-dimethyl-8- azabicyclo[3.2.1]octan-3- yl)oxy)pyridazin-3-yl)-6,7- dihydro-5H-pyrrolo[3,4-b]pyridin- 5-one |
| 213 | 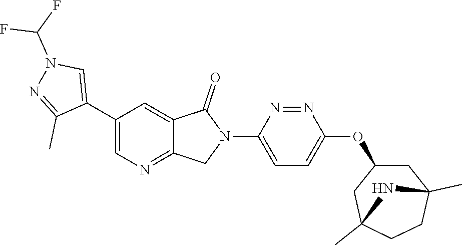 [SMILES: Cc1nn(C(F)F)cc1-c1cnc2c(c1)C(=O)N(c1ccc(O[C@H]3C[C@]4(C)CC[C@](C)(C3)N4)nn1)C2] | 3-(1-(difluoromethyl)-3-methyl- 1H-pyrazol-4-yl)-6-(6- (((1R,3s,5S)-1,5-dimethyl-8- azabicyclo[3.2.1]octan-3- yl)oxy)pyridazin-3-yl)-6,7- dihydro-5H-pyrrolo[3,4-b]pyridin- 5-one |
| 214 | 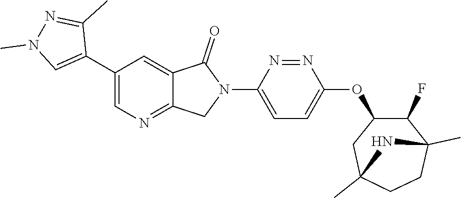 | 3-(1,3-dimethyl-1H-pyrazol-4-yl)- 6-(6-(((1S,2S,3R,5R)-2-fluoro-1,5- dimethyl-8- azabicyclo[3.2.1]octan-3- yl)oxy)pyridazin-3-yl)-6,7- dihydro-5H-pyrrolo[3,4-b]pyridin- 5-one |
| 215 | 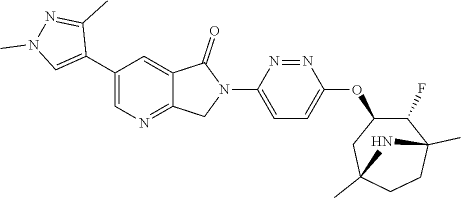 | 3-(1,3-dimethyl-1H-pyrazol-4-yl)- 6-(6-(((1S,2R,3R,5R)-2-fluoro- 1,5-dimethyl-8- azabicyclo[3.2.1]octan-3- yl)oxy)pyridazin-3-yl)-6,7- dihydro-5H-pyrrolo[3,4-b]pyridin- 5-one |
| 217 | 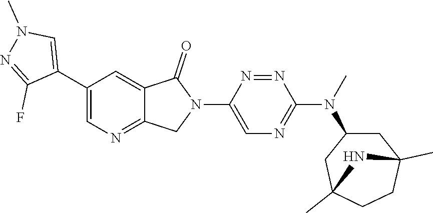 [SMILES: CN(c1ncc(N2Cc3ncc(-c4cn(C)nc4F)cc3C2=O)nn1)[C@H]1C[C@]2(C)CC[C@](C)(C1)N2] | 6-(3-(((1R,3s,5S)-1,5-dimethyl-8- azabicyclo[3.2.1]octan-3- yl)(methyl)amino)-1,2,4-triazin-6- yl)-3-(3-fluoro-1-methyl-1H- pyrazol-4-yl)-6,7-dihydro-5H- pyrrolo[3,4-b]pyridin-5-one |
| 219 | 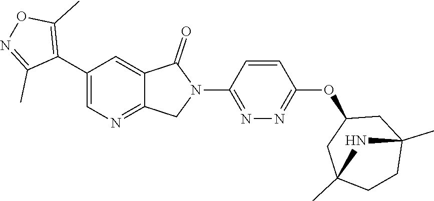 [SMILES: Cc1noc(C)c1-c1cnc2c(c1)C(=O)N(c1ccc(O[C@H]3C[C@]4(C)CC[C@](C)(C3)N4)nn1)C2] | 6-(6-(((1R,3s,5S)-1,5-dimethyl-8- azabicyclo[3.2.1]octan-3- yl)oxy)pyridazin-3-yl)-3-(3,5- dimethylisoxazol-4-yl)-6,7- dihydro-5H-pyrrolo[3,4-b]pyridin- 5-one |
| 220 | 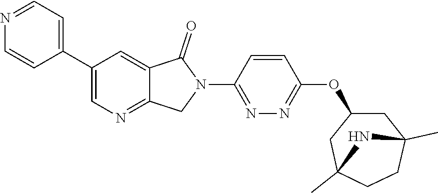 [SMILES: C[C@]12CC[C@](C)(C[C@@H](Oc3ccc(N4Cc5ncc(-c6ccncc6)cc5C4=O)nn3)C1)N2] | 6-(6-(((1R,3s,5S)-1,5-dimethyl-8- azabicyclo[3.2.1]octan-3- yl)oxy)pyridazin-3-yl)-3-(pyridin- 4-yl)-6,7-dihydro-5H-pyrrolo[3,4- b]pyridin-5-one |
| 227 | 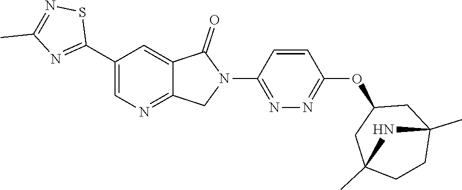 | 6-(6-(((1R,3s,5S)-1,5-dimethyl-8- azabicyclo[3.2.1]octan-3- yl)oxy)pyridazin-3-yl)-3-(3- methyl-1,2,4-thiadiazol-5-yl)-6,7- dihydro-5H-pyrrolo[3,4-b]pyridin- 5-one |
| 233 | 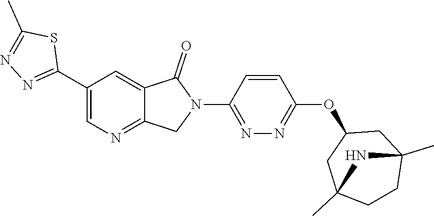 [SMILES: Cc1nnc(-c2cnc3c(c2)C(=O)N(c2ccc(O[C@H]4C[C@]5(C)CC[C@](C)(C4)N5)nn2)C3)s1] | 6-(6-(((1R,3s,5S)-1,5-dimethyl-8- azabicyclo[3.2.1]octan-3- yl)oxy)pyridazin-3-yl)-3-(5- methyl-1,3,4-thiadiazol-2-yl)-6,7- dihydro-5H-pyrrolo[3,4-b]pyridin- 5-one |
| 236 | 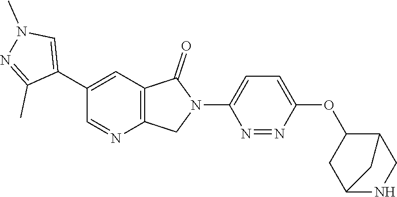 [SMILES: Cc1nn(C)cc1-c1cnc2c(c1)C(=O)N(c1ccc(OC3CC4CC3CN4)nn1)C2] | 6-(6-((2-azabicyclo[2.2.1]heptan- 5-yl)oxy)pyridazin-3-yl)-3-(1,3- dimethyl-1H-pyrazol-4-yl)-6,7- dihydro-5H-pyrrolo[3,4-b]pyridin- 5-one |
| 238 | 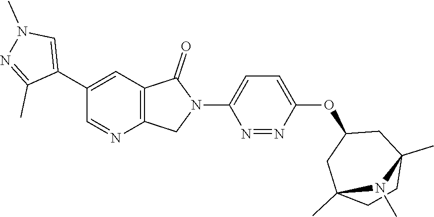 [SMILES: Cc1nn(C)cc1-c1cnc2c(c1)C(=O)N(c1ccc(O[C@H]3C[C@]4(C)CC[C@](C)(C3)N4C)nn1)C2] | 3-(1,3-dimethyl-1H-pyrazol-4-yl)- 6-(6-(((1R,3s,5S)-1,5,8-trimethyl- 8-azabicyclo[3.2.1]octan-3- yl)oxy)pyridazin-3-yl)-6,7- dihydro-5H-pyrrolo[3,4-b]pyridin- 5-one |
| 239 | 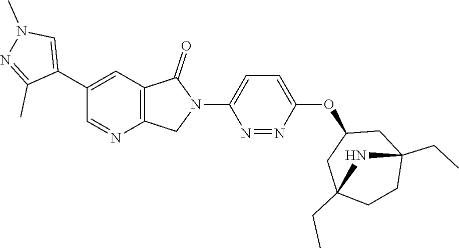 [SMILES: CC[C@]12CC[C@](CC)(C[C@@H](Oc3ccc(N4Cc5ncc(-c6cn(C)nc6C)cc5C4=O)nn3)C1)N2] | 6-(6-(((1R,3s,5S)-1,5-diethyl-8- azabicyclo[3.2.1]octan-3- yl)oxy)pyridazin-3-yl)-3-(1,3- dimethyl-1H-pyrazol-4-yl)-6,7- dihydro-5H-pyrrolo[3,4-b]pyridin- 5-one |
| 240 | 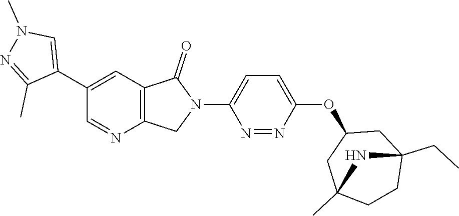 [SMILES: CC[C@]12CC[C@](C)(C[C@@H](Oc3ccc(N4Cc5ncc(-c6cn(C)nc6C)cc5C4=O)nn3)C1)N2] | 3-(1,3-dimethyl-1H-pyrazol-4-yl)- 6-(6-(((1S,3R,5R)-1-ethyl-5- methyl-8-azabicyclo[3.2.1]octan- 3-yl)oxy)pyridazin-3-yl)-6,7- dihydro-5H-pyrrolo[3,4-b]pyridin- 5-one |
| 242 | 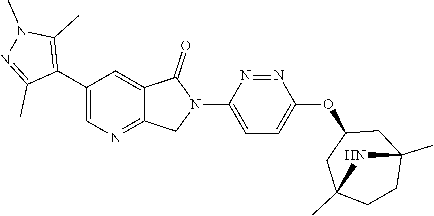 [SMILES: Cc1nn(C)c(C)c1-c1cnc2c(c1)C(=O)N(c1ccc(O[C@H]3C[C@]4(C)CC[C@](C)(C3)N4)nn1)C2] | 6-(6-(((1R,3s,5S)-1,5-dimethyl-8- azabicyclo[3.2.1]octan-3- yl)oxy)pyridazin-3-yl)-3-(1,3,5- trimethyl-1H-pyrazol-4-yl)-6,7- dihydro-5H-pyrrolo[3,4-b]pyridin- 5-one |
| 244 | 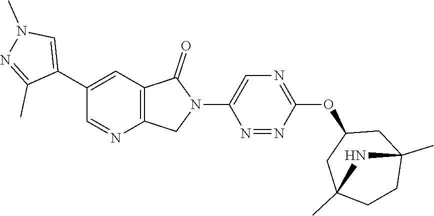 [SMILES: Cc1nn(C)cc1-c1cnc2c(c1)C(=O)N(c1cnc(O[C@H]3C[C@]4(C)CC[C@](C)(C3)N4)nn1)C2] | 3-(1,3-dimethyl-1H-pyrazol-4-yl)- 6-(3-(((1R,3s,5S)-1,5-dimethyl-8- azabicyclo[3.2.1]octan-3-yl)oxy)- 1,2,4-triazin-6-yl)-6,7-dihydro-5H- pyrrolo[3,4-b]pyridin-5-one |
| 246 | 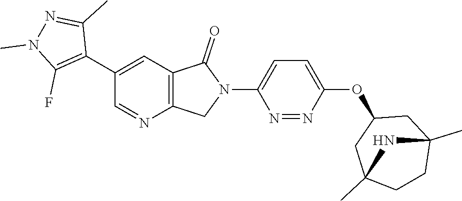 | 6-(6-(((1R,3s,5S)-1,5-dimethyl-8- azabicyclo[3.2.1]octan-3- yl)oxy)pyridazin-3-yl)-3-(5-fluoro- 1,3-dimethyl-1H-pyrazol-4-yl)- 6,7-dihydro-5H-pyrrolo[3,4- b]pyridin-5-one |
| 248 | 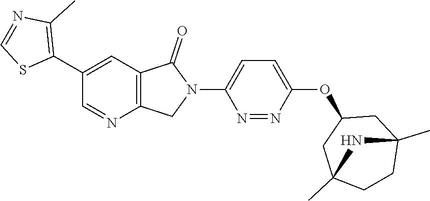 [SMILES: Cc1ncsc1-c1cnc2c(c1)C(=O)N(c1ccc(O[C@H]3C[C@]4(C)CC[C@](C)(C3)N4)nn1)C2] | 6-(6-(((1R,3s,5S)-1,5-dimethyl-8- azabicyclo[3.2.1]octan-3- yl)oxy)pyridazin-3-yl)-3-(4- methylthiazol-5-yl)-6,7-dihydro- 5H-pyrrolo[3,4-b]pyridin-5-one |
| 250 | 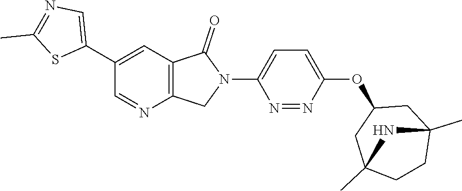 | 6-(6-(((1R,3s,5S)-1,5-dimethyl-8- azabicyclo[3.2.1]octan-3- yl)oxy)pyridazin-3-yl)-3-(2- methylthiazol-5-yl)-6,7-dihydro- 5H-pyrrolo[3,4-b]pyridin-5-one |
| 251 | 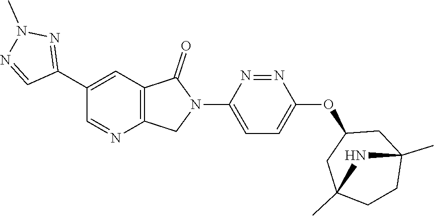 [SMILES: Cn1ncc(-c2cnc3c(c2)C(=O)N(c2ccc(O[C@H]4C[C@]5(C)CC[C@](C)(C4)N5)nn2)C3)n1] | 6-(6-(((1R,3s,5S)-1,5-dimethyl-8- azabicyclo[3.2.1]octan-3- yl)oxy)pyridazin-3-yl)-3-(2- methyl-2H-1,2,3-triazol-4-yl)-6,7- dihydro-5H-pyrrolo[3,4-b]pyridin- 5-one |
| 261 | 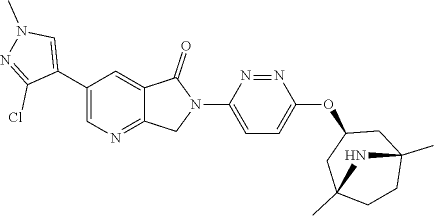 [SMILES: Cn1cc(-c2cnc3c(c2)C(=O)N(c2ccc(O[C@H]4C[C@]5(C)CC[C@](C)(C4)N5)nn2)C3)c(Cl)n1] | 3-(3-chloro-1-methyl-1H-pyrazol- 4-yl)-6-(6-(((1R,3s,5S)-1,5- dimethyl-8- azabicyclo[3.2.1]octan-3- yl)oxy)pyridazin-3-yl)-6,7- dihydro-5H-pyrrolo[3,4-b]pyridin- 5-one |
| 274 | 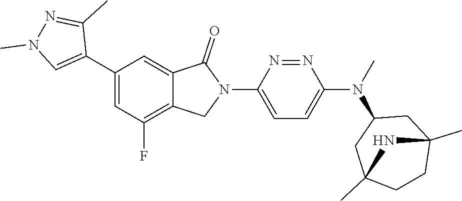 | 6-(1,3-dimethyl-1H-pyrazol-4-yl)- 2-(6-(((1R,3s,5S)-1,5-dimethyl-8- azabicyclo[3.2.1]octan-3- yl)(methyl)amino)pyridazin-3-yl)- 4-fluoroisoindolin-1-one |
| 278 | 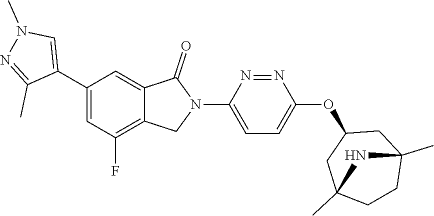 [SMILES: Cc1nn(C)cc1-c1cc(F)c2c(c1)C(=O)N(c1ccc(O[C@H]3C[C@]4(C)CC[C@](C)(C3)N4)nn1)C2] | 6-(1,3-dimethyl-1H-pyrazol-4-yl)- 2-(6-(((1R,3s,5S)-1,5-dimethyl-8- azabicyclo[3.2.1]octan-3- yl)oxy)pyridazin-3-yl)-4- fluoroisoindolin-1-one |
| 281 | 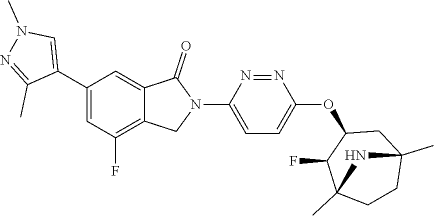 [SMILES: Cc1nn(C)cc1-c1cc(F)c2c(c1)C(=O)N(c1ccc(O[C@H]3C[C@]4(C)CC[C@@](C)(N4)[C@H]3F)nn1)C2] | 6-(1,3-dimethyl-1H-pyrazol-4-yl)- 4-fluoro-2-(6-(((1R,2R,3S,5S)-2- fluoro-1,5-dimethyl-8- azabicyclo[3.2.1]octan-3- yl)oxy)pyridazin-3-yl)isoindolin- 1-one |
| 283 | 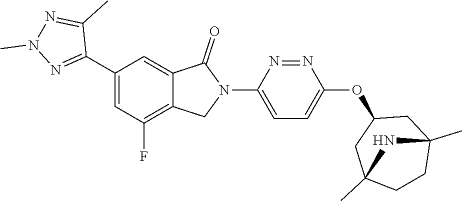 | 6-(2,5-dimethyl-2H-1,2,3-triazol- 4-yl)-2-(6-(((1R,3s,5S)-1,5- dimethyl-8- azabicyclo[3.2.1]octan-3- yl)oxy)pyridazin-3-yl)-4- fluoroisoindolin-1-one |
| 286 | 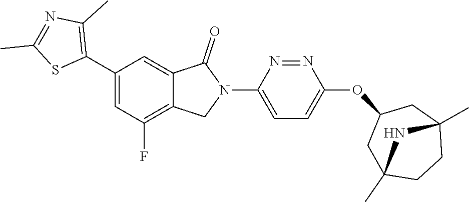 | 2-(6-(((1R,3s,5S)-1,5-dimethyl-8- azabicyclo[3.2.1]octan-3- yl)oxy)pyridazin-3-yl)-6-(2,4- dimethylthiazol-5-yl)-4- fluoroisoindolin-1-one |
| 289 | 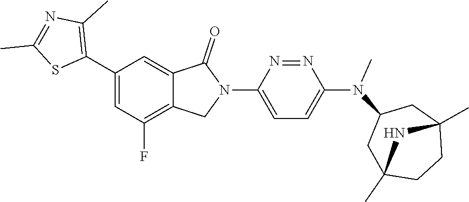 | 2-(6-(((1R,3s,5S)-1,5-dimethyl-8- azabicyclo[3.2.1]octan-3- yl)(methyl)amino)pyridazin-3-yl)- 6-(2,4-dimethylthiazol-5-yl)-4- fluoroisoindolin-1-one |
| 291 | 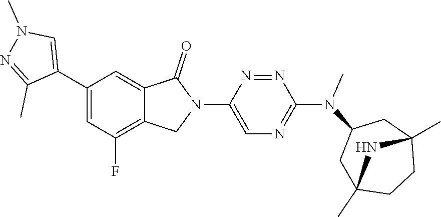 [SMILES: Cc1nn(C)cc1-c1cc(F)c2c(c1)C(=O)N(c1cnc(N(C)[C@H]3C[C@]4(C)CC[C@](C)(C3)N4)nn1)C2] | 6-(1,3-dimethyl-1H-pyrazol-4-yl)- 2-(3-(((1R,3s,5S)-1,5-dimethyl-8- azabicyclo[3.2.1]octan-3- yl)(methyl)amino)-1,2,4-triazin-6- yl)-4-fluoroisoindolin-1-one |
| 294 | 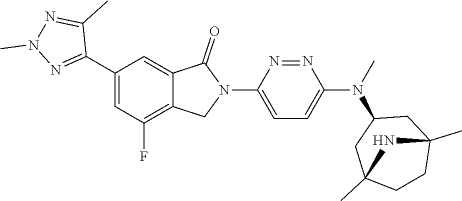 | 6-(2,5-dimethyl-2H-1,2,3-triazol- 4-yl)-2-(6-(((1R,3s,5S)-1,5- dimethyl-8- azabicyclo[3.2.1]octan-3- yl)(methyl)amino)pyridazin-3-yl)- 4-fluoroisoindolin-1-one |
| 303 | 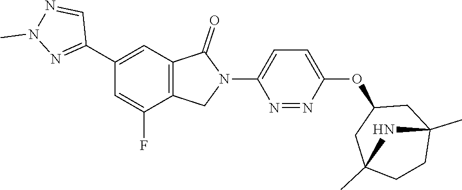 | 2-(6-(((1R,3s,5S)-1,5-dimethyl-8- azabicyclo[3.2.1]octan-3- yl)oxy)pyridazin-3-yl)-4-fluoro-6- (2-methyl-2H-1,2,3-triazol-4- yl)isoindolin-1-one |
| 366 | 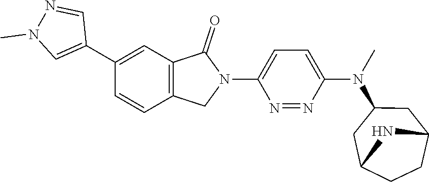 [SMILES: CN(c1ccc(N2Cc3ccc(-c4cnn(C)c4)cc3C2=O)nn1)[C@@H]1C[C@H]2CC[C@@H](C1)N2] | 2-(6-(((1R,3s,5S)-8- azabicyclo[3.2.1]octan-3- yl)(methyl)amino)pyridazin-3-yl)- 6-(1-methyl-1H-pyrazol-4- yl)isoindolin-1-one |
| 367 | 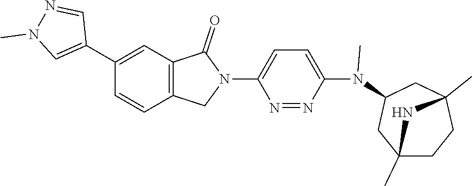 | 2-(6-(((1R,3s,5S)-1,5-dimethyl-8- azabicyclo[3.2.1]octan-3- yl)(methyl)amino)pyridazin-3-yl)- 6-(1-methyl-1H-pyrazol-4- yl)isoindolin-1-one |
| 376 | 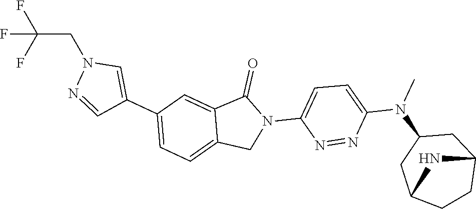 [SMILES: CN(c1ccc(N2Cc3ccc(-c4cnn(CC(F)(F)F)c4)cc3C2=O)nn1)[C@@H]1C[C@H]2CC[C@@H](C1)N2] | 2-(6-(((1R,3s,5S)-8- azabicyclo[3.2.1]octan-3- yl)(methyl)amino)pyridazin-3-yl)- 6-(1-(2,2,2-trifluoroethyl)-1H- pyrazol-4-yl)isoindolin-1-one |
| 377 | 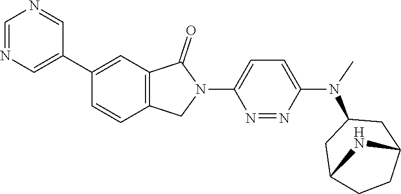 [SMILES: CN(c1ccc(N2Cc3ccc(-c4cncnc4)cc3C2=O)nn1)[C@@H]1C[C@H]2CC[C@@H](C1)N2] | 2-(6-(((1R,3s,5S)-8- azabicyclo[3.2.1]octan-3- yl)(methyl)amino)pyridazin-3-yl)- 6-(pyrimidin-5-yl)isoindolin-1-one |
| 386 | 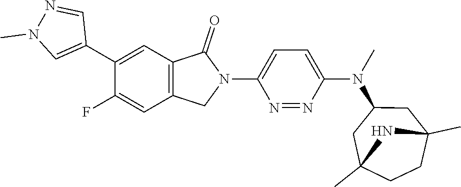 | 2-(6-(((1R,3s,5S)-1,5-dimethyl-8- azabicyclo[3.2.1]octan-3- yl)(methyl)amino)pyridazin-3-yl)- 5-fluoro-6-(1-methyl-1H-pyrazol- 4-yl)isoindolin-1-one |
| 387 | 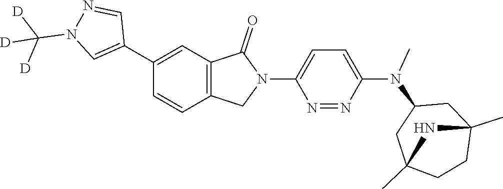 [SMILES: [2H]C([2H])([2H])n1cc(-c2ccc3c(c2)C(=O)N(c2ccc(N(C)[C@H]4C[C@]5(C)CC[C@](C)(C4)N5)nn2)C3)cn1] | 2-(6-(((1R,3s,5S)-1,5-dimethyl-8- azabicyclo[3.2.1]octan-3- yl)(methyl)amino)pyridazin-3-yl)- 6-(1-(methyl-d3)-1H-pyrazol-4- yl)isoindolin-1-one |
| 396 | 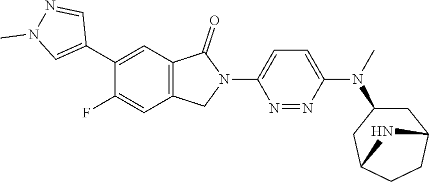 [SMILES: CN(c1ccc(N2Cc3cc(F)c(-c4cnn(C)c4)cc3C2=O)nn1)[C@@H]1C[C@H]2CC[C@@H](C1)N2] | 2-(6-(((1R,3s,5S)-8- azabicyclo[3.2.1]octan-3- yl)(methyl)amino)pyridazin-3-yl)- 5-fluoro-6-(1-methyl-1H-pyrazol- 4-yl)isoindolin-1-one |
| 397 | 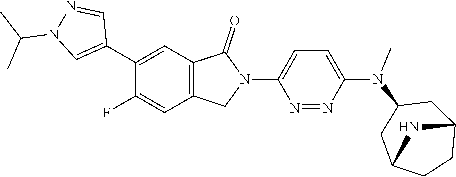 [SMILES: CC(C)n1cc(-c2cc3c(cc2F)CN(c2ccc(N(C)[C@@H]4C[C@H]5CC[C@@H](C4)N5)nn2)C3=O)cn1] | 2-(6-(((1R,3s,5S)-8- azabicyclo[3.2.1]octan-3- yl)(methyl)amino)pyridazin-3-yl)- 5-fluoro-6-(1-isopropyl-1H- pyrazol-4-yl)isoindolin-1-one |
| 398 | 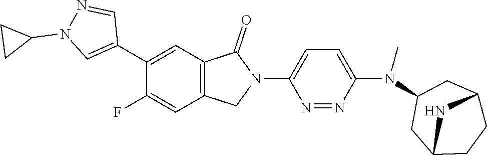 [SMILES: CN(c1ccc(N2Cc3cc(F)c(-c4cnn(C5CC5)c4)cc3C2=O)nn1)[C@@H]1C[C@H]2CC[C@@H](C1)N2] | 2-(6-(((1R,3s,5S)-8- azabicyclo[3.2.1]octan-3- yl)(methyl)amino)pyridazin-3-yl)- 6-(1-cyclopropyl-1H-pyrazol-4- yl)-5-fluoroisoindolin-1-one |
| 413 | 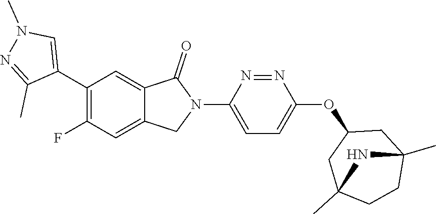 [SMILES: Cc1nn(C)cc1-c1cc2c(cc1F)CN(c1ccc(O[C@H]3C[C@]4(C)CC[C@](C)(C3)N4)nn1)C2=O] | 6-(1,3-dimethyl-1H-pyrazol-4-yl)- 2-(6-(((1R,3s,5S)-1,5-dimethyl-8- azabicyclo[3.2.1]octan-3- yl)oxy)pyridazin-3-yl)-5- fluoroisoindolin-1-one |
| 415 | 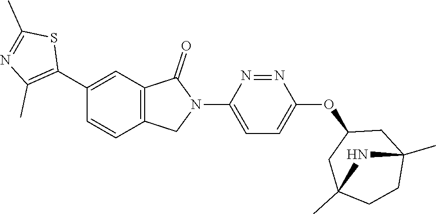 [SMILES: Cc1nc(C)c(-c2ccc3c(c2)C(=O)N(c2ccc(O[C@H]4C[C@]5(C)CC[C@](C)(C4)N5)nn2)C3)s1] | 2-(6-(((1R,3s,5S)-1,5-dimethyl-8- azabicyclo[3.2.1]octan-3- yl)oxy)pyridazin-3-yl)-6-(2,4- dimethylthiazol-5-yl)isoindolin-1- one |
| 416 | 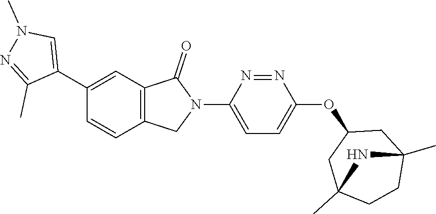 [SMILES: Cc1nn(C)cc1-c1ccc2c(c1)C(=O)N(c1ccc(O[C@H]3C[C@]4(C)CC[C@](C)(C3)N4)nn1)C2] | 6-(1,3-dimethyl-1H-pyrazol-4-yl)- 2-(6-(((1R,3s,5S)-1,5-dimethyl-8- azabicyclo[3.2.1]octan-3- yl)oxy)pyridazin-3-yl)isoindolin- 1-one |
| 418 | 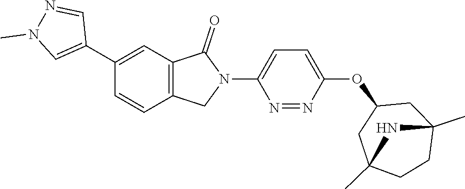 | 2-(6-(((1R,3s,5S)-1,5-dimethyl-8- azabicyclo[3.2.1]octan-3- yl)oxy)pyridazin-3-yl)-6-(1- methyl-1H-pyrazol-4- yl)isoindolin-1-one |
| 419 | 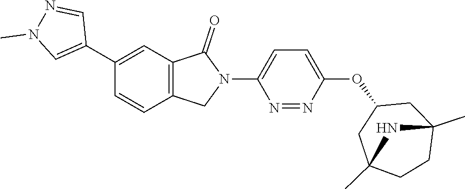 | 2-(6-(((1R,3r,5S)-1,5-dimethyl-8- azabicyclo[3.2.1]octan-3- yl)oxy)pyridazin-3-yl)-6-(1- methyl-1H-pyrazol-4- yl)isoindolin-1-one |
| 422 | 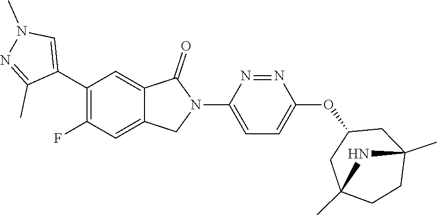 [SMILES: Cc1nn(C)cc1-c1cc2c(cc1F)CN(c1ccc(O[C@@H]3C[C@]4(C)CC[C@](C)(C3)N4)nn1)C2=O] | 6-(1,3-dimethyl-1H-pyrazol-4-yl)- 2-(6-(((1R,3r,5S)-1,5-dimethyl-8- azabicyclo[3.2.1]octan-3- yl)oxy)pyridazin-3-yl)-5- fluoroisoindolin-1-one |
| 423 | 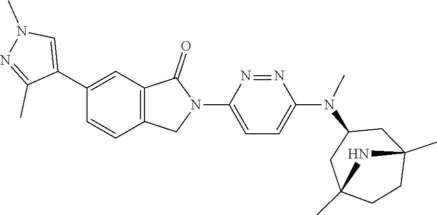 [SMILES: Cc1nn(C)cc1-c1ccc2c(c1)C(=O)N(c1ccc(N(C)[C@H]3C[C@]4(C)CC[C@](C)(C3)N4)nn1)C2] | 6-(1,3-dimethyl-1H-pyrazol-4-yl)- 2-(6-(((1R,3s,5S)-1,5-dimethyl-8- azabicyclo[3.2.1]octan-3- yl)(methyl)amino)pyridazin-3- yl)isoindolin-1-one |
| 425 | 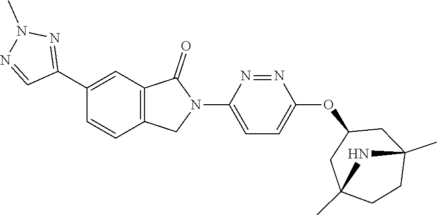 [SMILES: Cn1ncc(-c2ccc3c(c2)C(=O)N(c2ccc(O[C@H]4C[C@]5(C)CC[C@](C)(C4)N5)nn2)C3)n1] | 2-(6-(((1R,3s,5S)-1,5-dimethyl-8- azabicyclo[3.2.1]octan-3- yl)oxy)pyridazin-3-yl)-6-(2- methyl-2H-1,2,3-triazol-4- yl)isoindolin-1-one |
| 426 | 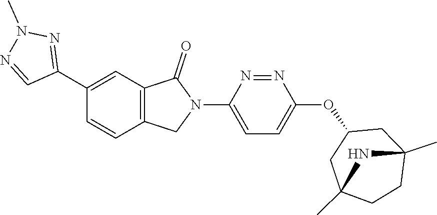 [SMILES: Cn1ncc(-c2ccc3c(c2)C(=O)N(c2ccc(O[C@@H]4C[C@]5(C)CC[C@](C)(C4)N5)nn2)C3)n1] | 2-(6-(((1R,3r,5S)-1,5-dimethyl-8- azabicyclo[3.2.1]octan-3- yl)oxy)pyridazin-3-yl)-6-(2- methyl-2H-1,2,3-triazol-4- yl)isoindolin-1-one |
| 427 | 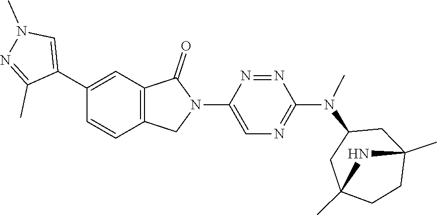 [SMILES: Cc1nn(C)cc1-c1ccc2c(c1)C(=O)N(c1cnc(N(C)[C@H]3C[C@]4(C)CC[C@](C)(C3)N4)nn1)C2] | 6-(1,3-dimethyl-1H-pyrazol-4-yl)- 2-(3-(((1R,3s,5S)-1,5-dimethyl-8- azabicyclo[3.2.1]octan-3- yl)(methyl)amino)-1,2,4-triazin-6- yl)isoindolin-1-one |
| 428 | 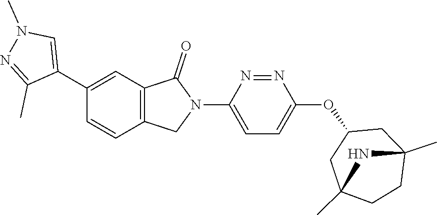 [SMILES: Cc1nn(C)cc1-c1ccc2c(c1)C(=O)N(c1ccc(O[C@@H]3C[C@]4(C)CC[C@](C)(C3)N4)nn1)C2] | 6-(1,3-dimethyl-1H-pyrazol-4-yl)- 2-(6-(((1R,3r,5S)-1,5-dimethyl-8- azabicyclo[3.2.1]octan-3- yl)oxy)pyridazin-3-yl)isoindolin- 1-one |
| 429 | 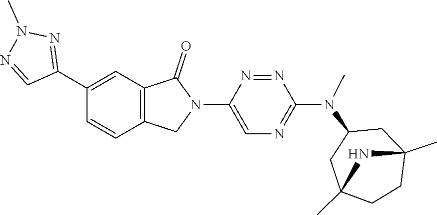 [SMILES: CN(c1ncc(N2Cc3ccc(-c4cnn(C)n4)cc3C2=O)nn1)[C@H]1C[C@]2(C)CC[C@](C)(C1)N2] | 2-(3-(((1R,3s,5S)-1,5-dimethyl-8- azabicyclo[3.2.1]octan-3- yl)(methyl)amino)-1,2,4-triazin-6- yl)-6-(2-methyl-2H-1,2,3-triazol- 4-yl)isoindolin-1-one |
| 432 | 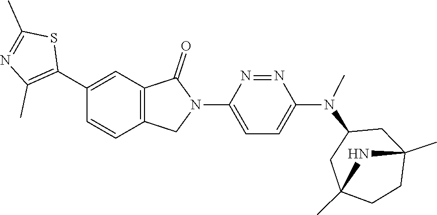 [SMILES: Cc1nc(C)c(-c2ccc3c(c2)C(=O)N(c2ccc(N(C)[C@H]4C[C@]5(C)CC[C@](C)(C4)N5)nn2)C3)s1] | 2-(6-(((1R,3s,5S)-1,5-dimethyl-8- azabicyclo[3.2.1]octan-3- yl)(methyl)amino)pyridazin-3-yl)- 6-(2,4-dimethylthiazol-5- yl)isoindolin-1-one |
| 434 | 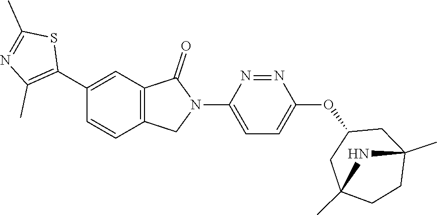 [SMILES: Cc1nc(C)c(-c2ccc3c(c2)C(=O)N(c2ccc(O[C@@H]4C[C@]5(C)CC[C@](C)(C4)N5)nn2)C3)s1] | 2-(6-(((1R,3r,5S)-1,5-dimethyl-8- azabicyclo[3.2.1]octan-3- yl)oxy)pyridazin-3-yl)-6-(2,4- dimethylthiazol-5-yl)isoindolin-1- one |
| 436 | 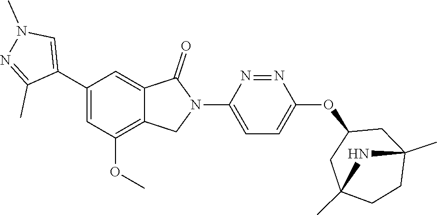 [SMILES: COc1cc(-c2cn(C)nc2C)cc2c1CN(c1ccc(O[C@H]3C[C@]4(C)CC[C@](C)(C3)N4)nn1)C2=O] | 6-(1,3-dimethyl-1H-pyrazol-4-yl)- 2-(6-(((1R,3s,5S)-1,5-dimethyl-8- azabicyclo[3.2.1]octan-3- yl)oxy)pyridazin-3-yl)-4- methoxyisoindolin-1-one |
| 437 | 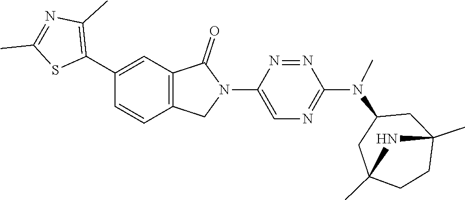 | 2-(3-(((1R,3s,5S)-1,5-dimethyl-8- azabicyclo[3.2.1]octan-3- yl)(methyl)amino)-1,2,4-triazin-6- yl)-6-(2,4-dimethylthiazol-5- yl)isoindolin-1-one |
| 439 | 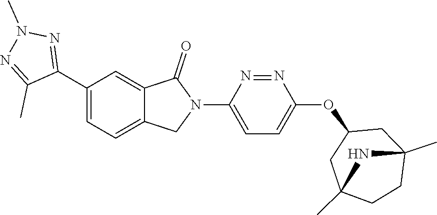 [SMILES: Cc1nn(C)nc1-c1ccc2c(c1)C(=O)N(c1ccc(O[C@H]3C[C@]4(C)CC[C@](C)(C3)N4)nn1)C2] | 6-(2,5-dimethyl-2H-1,2,3-triazol- 4-yl)-2-(6-(((1R,3s,5S)-1,5- dimethyl-8- azabicyclo[3.2.1]octan-3- yl)oxy)pyridazin-3-yl)isoindolin- 1-one |
| 445 | 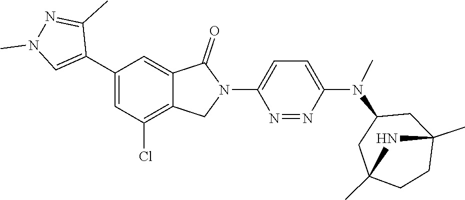 | 4-chloro-6-(1,3-dimethyl-1H- pyrazol-4-yl)-2-(6-(((1R,3s,5S)- 1,5-dimethyl-8- azabicyclo[3.2.1]octan-3- yl)(methyl)amino)pyridazin-3- yl)isoindolin-1-one |
| 446 | 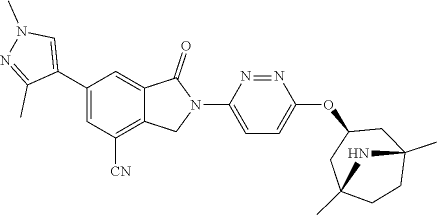 [SMILES: Cc1nn(C)cc1-c1cc(C#N)c2c(c1)C(=O)N(c1ccc(O[C@H]3C[C@]4(C)CC[C@](C)(C3)N4)nn1)C2] | 6-(1,3-dimethyl-1H-pyrazol-4-yl)- 2-(6-(((1R,3s,5S)-1,5-dimethyl-8- azabicyclo[3.2.1]octan-3- yl)oxy)pyridazin-3-yl)-1- oxoisoindoline-4-carbonitrile |
| 447 | 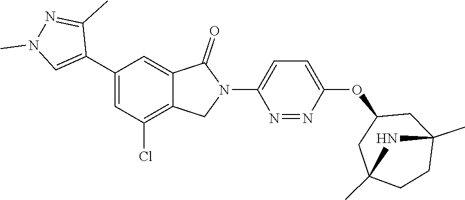 | 4-chloro-6-(1,3-dimethyl-1H- pyrazol-4-yl)-2-(6-(((1R,3s,5S)- 1,5-dimethyl-8- azabicyclo[3.2.1]octan-3- yl)oxy)pyridazin-3-yl)isoindolin- 1-one |
| 452 | 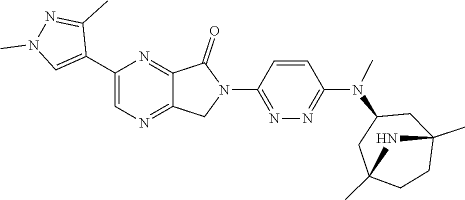 | 3-(1,3-dimethyl-1H-pyrazol-4-yl)- 6-(6-(((1R,3s,5S)-1,5-dimethyl-8- azabicyclo[3.2.1]octan-3- yl)(methyl)amino)pyridazin-3-yl)- 6,7-dihydro-5H-pyrrolo[3,4- b]pyrazin-5-one |
| 453 | 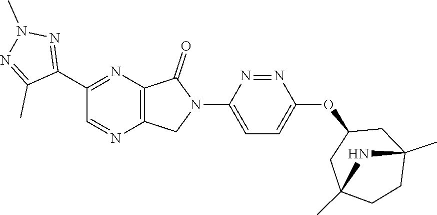 [SMILES: Cc1nn(C)nc1-c1cnc2c(n1)C(=O)N(c1ccc(O[C@H]3C[C@]4(C)CC[C@](C)(C3)N4)nn1)C2] | 3-(2,5-dimethyl-2H-1,2,3-triazol- 4-yl)-6-(6-(((1R,3s,5S)-1,5- dimethyl-8- azabicyclo[3.2.1]octan-3- yl)oxy)pyridazin-3-yl)-6,7- dihydro-5H-pyrrolo[3,4-b]pyrazin- 5-one |
| 454 | 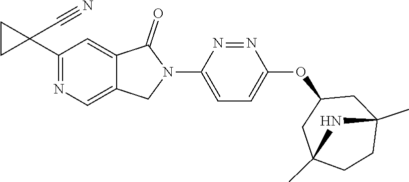 [SMILES: C[C@]12CC[C@](C)(C[C@@H](Oc3ccc(N4Cc5cnc(C6(C#N)CC6)cc5C4=O)nn3)C1)N2] | 1-(2-(6-(((1R,3s,5S)-1,5-dimethyl- 8-azabicyclo[3.2.1]octan-3- yl)oxy)pyridazin-3-yl)-1-oxo-2,3- dihydro-1H-pyrrolo[3,4-c]pyridin- 6-yl)cyclopropane-1-carbonitrile |
| 456 | 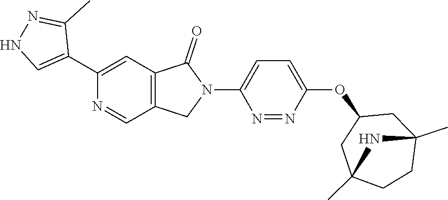 [SMILES: Cc1n[nH]cc1-c1cc2c(cn1)CN(c1ccc(O[C@H]3C[C@]4(C)CC[C@](C)(C3)N4)nn1)C2=O] | 2-(6-(((1R,3s,5S)-1,5-dimethyl-8- azabicyclo[3.2.1]octan-3- yl)oxy)pyridazin-3-yl)-6-(3- methyl-1H-pyrazol-4-yl)-2,3- dihydro-1H-pyrrolo[3,4-c]pyridin- 1-one |
| 458 | 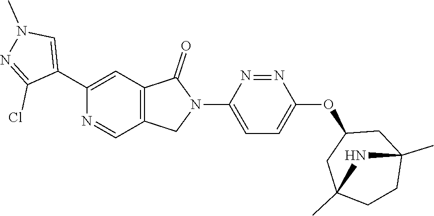 [SMILES: Cn1cc(-c2cc3c(cn2)CN(c2ccc(O[C@H]4C[C@]5(C)CC[C@](C)(C4)N5)nn2)C3=O)c(Cl)n1] | 6-(3-chloro-1-methyl-1H-pyrazol- 4-yl)-2-(6-(((1R,3s,5S)-1,5- dimethyl-8- azabicyclo[3.2.1]octan-3- yl)oxy)pyridazin-3-yl)-2,3- dihydro-1H-pyrrolo[3,4-c]pyridin- 1-one |
| 460 | 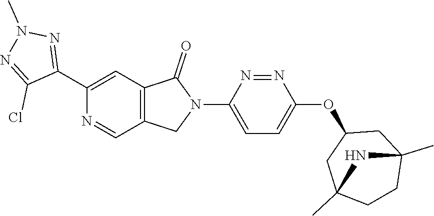 [SMILES: Cn1nc(Cl)c(-c2cc3c(cn2)CN(c2ccc(O[C@H]4C[C@]5(C)CC[C@](C)(C4)N5)nn2)C3=O)n1] | 6-(5-chloro-2-methyl-2H-1,2,3- triazol-4-yl)-2-(6-(((1R,3s,5S)-1,5- dimethyl-8- azabicyclo[3.2.1]octan-3- yl)oxy)pyridazin-3-yl)-2,3- dihydro-1H-pyrrolo[3,4-c]pyridin- 1-one |
| 467 | 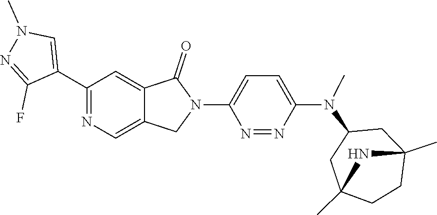 [SMILES: CN(c1ccc(N2Cc3cnc(-c4cn(C)nc4F)cc3C2=O)nn1)[C@H]1C[C@]2(C)CC[C@](C)(C1)N2] | 2-(6-(((1R,3s,5S)-1,5-dimethyl-8- azabicyclo[3.2.1]octan-3- yl)(methyl)amino)pyridazin-3-yl)- 6-(3-fluoro-1-methyl-1H-pyrazol- 4-yl)-2,3-dihydro-1H-pyrrolo[3,4- c]pyridin-1-one |
| 470 | 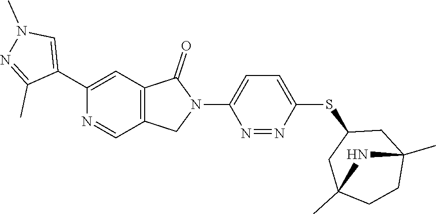 [SMILES: Cc1nn(C)cc1-c1cc2c(cn1)CN(c1ccc(S[C@H]3C[C@]4(C)CC[C@](C)(C3)N4)nn1)C2=O] | 6-(1,3-dimethyl-1H-pyrazol-4-yl)- 2-(6-(((1R,3s,5S)-1,5-dimethyl-8- azabicyclo[3.2.1]octan-3- yl)thio)pyridazin-3-yl)-2,3- dihydro-1H-pyrrolo[3,4-c]pyridin- 1-one |
| 477 | 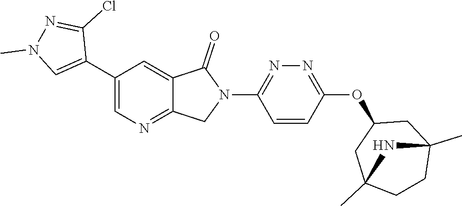 | 3-(3-chloro-1-methyl-1H-pyrazol- 4-yl)-6-(6-(((1R,3s,5S)-1,5- dimethyl-8- azabicyclo[3.2.1]octan-3- yl)oxy)pyridazin-3-yl)-6,7- dihydro-5H-pyrrolo[3,4-b]pyridin- 5-one |
| 478 | 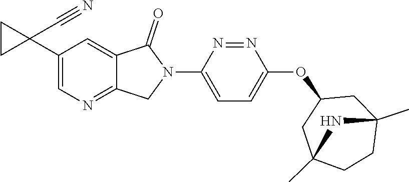 [SMILES: C[C@]12CC[C@](C)(C[C@@H](Oc3ccc(N4Cc5ncc(C6(C#N)CC6)cc5C4=O)nn3)C1)N2] | 1-(6-(6-(((1R,3s,5S)-1,5-dimethyl- 8-azabicyclo[3.2.1]octan-3- yl)oxy)pyridazin-3-yl)-5-oxo-6,7- dihydro-5H-pyrrolo[3,4-b]pyridin- 3-yl)cyclopropane-1-carbonitrile |
| 480 | 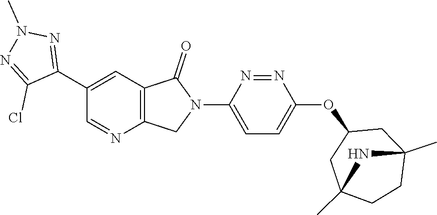 [SMILES: Cn1nc(Cl)c(-c2cnc3c(c2)C(=O)N(c2ccc(O[C@H]4C[C@]5(C)CC[C@](C)(C4)N5)nn2)C3)n1] | 3-(5-chloro-2-methyl-2H-1,2,3- triazol-4-yl)-6-(6-(((1R,3s,5S)-1,5- dimethyl-8- azabicyclo[3.2.1]octan-3- yl)oxy)pyridazin-3-yl)-6,7- dihydro-5H-pyrrolo[3,4-b]pyridin- 5-one |
| 490 | 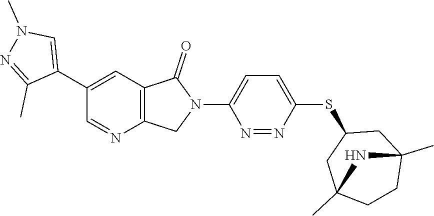 [SMILES: Cc1nn(C)cc1-c1cnc2c(c1)C(=O)N(c1ccc(S[C@H]3C[C@]4(C)CC[C@](C)(C3)N4)nn1)C2] | 3-(1,3-dimethyl-1H-pyrazol-4-yl)- 6-(6-(((1R,3s,5S)-1,5-dimethyl-8- azabicyclo[3.2.1]octan-3- yl)thio)pyridazin-3-yl)-6,7- dihydro-5H-pyrrolo[3,4-b]pyridin- 5-one |
| 495 | 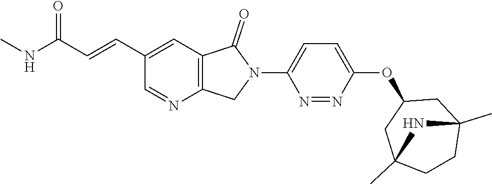 | (E)-3-(6-(6-(((1R,3s,5S)-1,5- dimethyl-8- azabicyclo[3.2.1]octan-3- yl)oxy)pyridazin-3-yl)-5-oxo-6,7- dihydro-5H-pyrrolo[3,4-b]pyridin- 3-yl)-N-methylacrylamide |
| 496 | 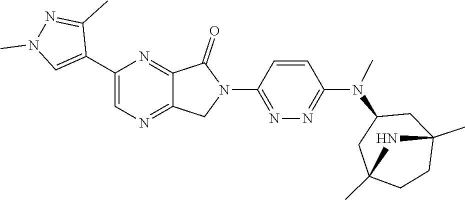 | 3-(1,3-dimethyl-1H-pyrazol-4-yl)- 6-(6-(((1R,3s,5S)-1,5-dimethyl-8- azabicyclo[3.2.1]octan-3- yl)(methyl)amino)pyridazin-3-yl)- 6,7-dihydro-5H-pyrrolo[3,4- b]pyrazin-5-one |
| 498 | 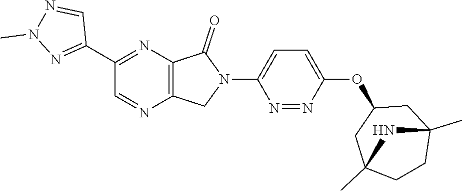 | 6-(6-(((1R,3s,5S)-1,5-dimethyl-8- azabicyclo[3.2.1]octan-3- yl)oxy)pyridazin-3-yl)-3-(2- methyl-2H-1,2,3-triazol-4-yl)-6,7- dihydro-5H-pyrrolo[3,4-b]pyrazin- 5-one |
| 502 | 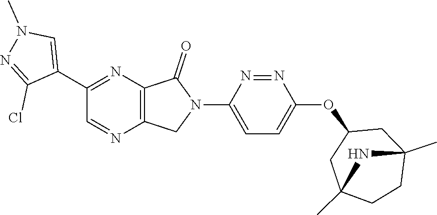 [SMILES: Cn1cc(-c2cnc3c(n2)C(=O)N(c2ccc(O[C@H]4C[C@]5(C)CC[C@](C)(C4)N5)nn2)C3)c(Cl)n1] | 3-(3-chloro-1-methyl-1H-pyrazol- 4-yl)-6-(6-(((1R,3s,5S)-1,5- dimethyl-8- azabicyclo[3.2.1]octan-3- yl)oxy)pyridazin-3-yl)-6,7- dihydro-5H-pyrrolo[3,4-b]pyrazin- 5-one |
| 503 | 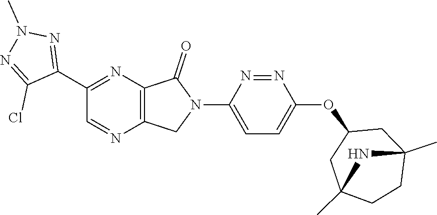 [SMILES: Cn1nc(Cl)c(-c2cnc3c(n2)C(=O)N(c2ccc(O[C@H]4C[C@]5(C)CC[C@](C)(C4)N5)nn2)C3)n1] | 3-(5-chloro-2-methyl-2H-1,2,3- triazol-4-yl)-6-(6-(((1R,3s,5S)-1,5- dimethyl-8- azabicyclo[3.2.1]octan-3- yl)oxy)pyridazin-3-yl)-6,7- dihydro-5H-pyrrolo[3,4-b]pyrazin- 5-one |
| 589 | 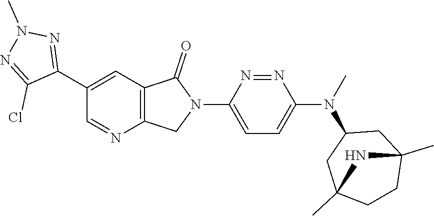 [SMILES: CN(c1ccc(N2Cc3ncc(-c4nn(C)nc4Cl)cc3C2=O)nn1)[C@H]1C[C@]2(C)CC[C@](C)(C1)N2] | 3-(5-chloro-2-methyl-2H-1,2,3- triazol-4-yl)-6-(6-(((1R,3s,5S)-1,5- dimethyl-8- azabicyclo[3.2.1]octan-3- yl)(methyl)amino)pyridazin-3-yl)- 6,7-dihydro-5H-pyrrolo[3,4- b]pyridin-5-one |
| 590 | 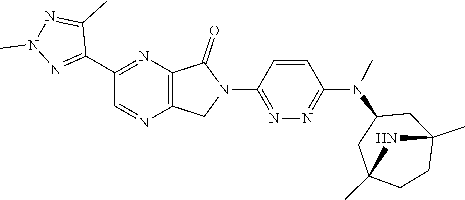 | 3-(2,5-dimethyl-2H-1,2,3-triazol- 4-yl)-6-(6-(((1R,3s,5S)-1,5- dimethyl-8- azabicyclo[3.2.1]octan-3- yl)(methyl)amino)pyridazin-3-yl)- 6,7-dihydro-5H-pyrrolo[3,4- b]pyrazin-5-one |
| 591 | 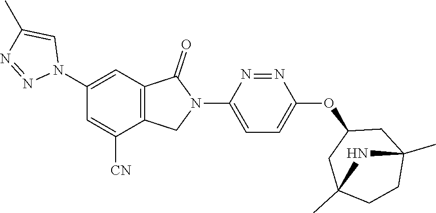 [SMILES: Cc1cn(-c2cc(C#N)c3c(c2)C(=O)N(c2ccc(O[C@H]4C[C@]5(C)CC[C@](C)(C4)N5)nn2)C3)nn1] | 2-(6-(((1R,3s,5S)-1,5-dimethyl-8- azabicyclo[3.2.1]octan-3- yl)oxy)pyridazin-3-yl)-6-(4- methyl-1H-1,2,3-triazol-1-yl)-1- oxoisoindoline-4-carbonitrile |
| 592 | 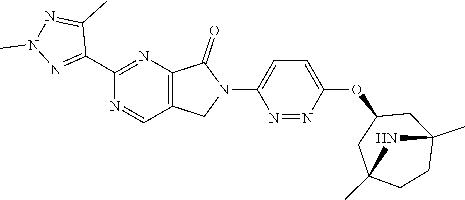 | 2-(2,5-dimethyl-2H-1,2,3-triazol- 4-yl)-6-(6-(((1R,3s,5S)-1,5- dimethyl-8- azabicyclo[3.2.1]octan-3- yl)oxy)pyridazin-3-yl)-5,6- dihydro-7H-pyrrolo[3,4- d]pyrimidin-7-one |
| 594 | 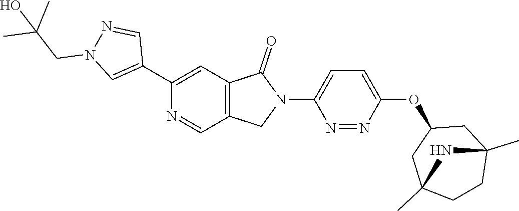 [SMILES: CC(C)(O)Cn1cc(-c2cc3c(cn2)CN(c2ccc(O[C@H]4C[C@]5(C)CC[C@](C)(C4)N5)nn2)C3=O)cn1] | 2-(6-(((1R,3s,5S)-1,5-dimethyl-8- azabicyclo[3.2.1]octan-3- yl)oxy)pyridazin-3-yl)-6-(1-(2- hydroxy-2-methylpropyl)-1H- pyrazol-4-yl)-2,3-dihydro-1H- pyrrolo[3,4-c]pyridin-1-one |
| 595 | 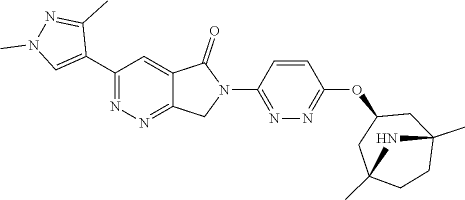 | 3-(1,3-dimethyl-1H-pyrazol-4-yl)- 6-(6-(((1R,3s,5S)-1,5-dimethyl-8- azabicyclo[3.2.1]octan-3- yl)oxy)pyridazin-3-yl)-6,7- dihydro-5H-pyrrolo[3,4- c]pyridazin-5-one |
| 596 | 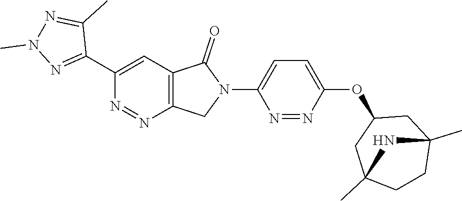 | 3-(2,5-dimethyl-2H-1,2,3-triazol- 4-yl)-6-(6-(((1R,3s,5S)-1,5- dimethyl-8- azabicyclo[3.2.1]octan-3- yl)oxy)pyridazin-3-yl)-6,7- dihydro-5H-pyrrolo[3,4- c]pyridazin-5-one |
| 598 | 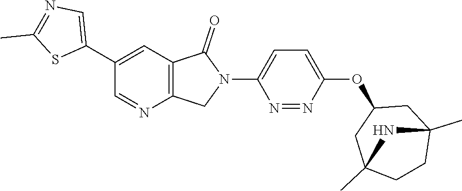 | 6-(6-(((1R,3s,5S)-1,5-dimethyl-8- azabicyclo[3.2.1]octan-3- yl)oxy)pyridazin-3-yl)-3-(2- methylthiazol-5-yl)-6,7-dihydro- 5H-pyrrolo[3,4-b]pyridin-5-one |
| 599 | 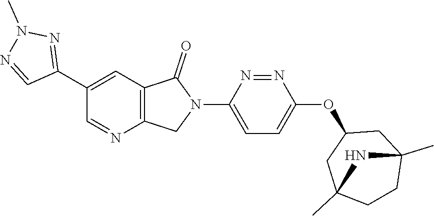 [SMILES: Cn1ncc(-c2cnc3c(c2)C(=O)N(c2ccc(O[C@H]4C[C@]5(C)CC[C@](C)(C4)N5)nn2)C3)n1] | 6-(6-(((1R,3s,5S)-1,5-dimethyl-8- azabicyclo[3.2.1]octan-3- yl)oxy)pyridazin-3-yl)-3-(2- methyl-2H-1,2,3-triazol-4-yl)-6,7- dihydro-5H-pyrrolo[3,4-b]pyridin- 5-one |
| 604 | 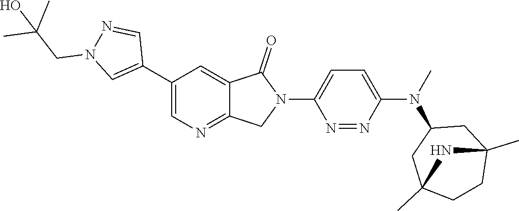 [SMILES: CN(c1ccc(N2Cc3ncc(-c4cnn(CC(C)(C)O)c4)cc3C2=O)nn1)[C@H]1C[C@]2(C)CC[C@](C)(C1)N2] | 6-(6-(((1R,3s,5S)-1,5-dimethyl-8- azabicyclo[3.2.1]octan-3- yl)(methyl)amino)pyridazin-3-yl)- 3-(1-(2-hydroxy-2-methylpropyl)- 1H-pyrazol-4-yl)-6,7-dihydro-5H- pyrrolo[3,4-b]pyridin-5-one |
| 606 | 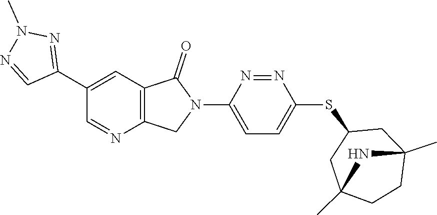 [SMILES: Cn1ncc(-c2cnc3c(c2)C(=O)N(c2ccc(S[C@H]4C[C@]5(C)CC[C@](C)(C4)N5)nn2)C3)n1] | 6-(6-(((1R,3s,5S)-1,5-dimethyl-8- azabicyclo[3.2.1]octan-3- yl)thio)pyridazin-3-yl)-3-(2- methyl-2H-1,2,3-triazol-4-yl)-6,7- dihydro-5H-pyrrolo[3,4-b]pyridin- 5-one |
| 609 | 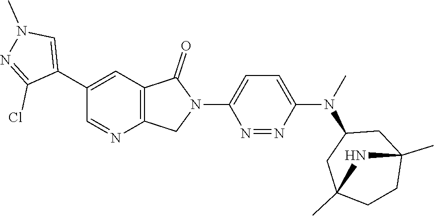 [SMILES: CN(c1ccc(N2Cc3ncc(-c4cn(C)nc4Cl)cc3C2=O)nn1)[C@H]1C[C@]2(C)CC[C@](C)(C1)N2] | 3-(3-chloro-1-methyl-1H-pyrazol- 4-yl)-6-(6-(((1R,3s,5S)-1,5- dimethyl-8- azabicyclo[3.2.1]octan-3- yl)(methyl)amino)pyridazin-3-yl)- 6,7-dihydro-5H-pyrrolo[3,4- b]pyridin-5-one |
| 610 | 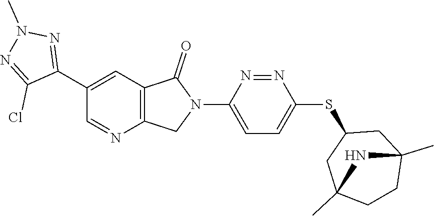 [SMILES: Cn1nc(Cl)c(-c2cnc3c(c2)C(=O)N(c2ccc(S[C@H]4C[C@]5(C)CC[C@](C)(C4)N5)nn2)C3)n1] | 3-(5-chloro-2-methyl-2H-1,2,3- triazol-4-yl)-6-(6-(((1R,3s,5S)-1,5- dimethyl-8- azabicyclo[3.2.1]octan-3- yl)thio)pyridazin-3-yl)-6,7- dihydro-5H-pyrrolo[3,4-b]pyridin- 5-one |
| 611 | 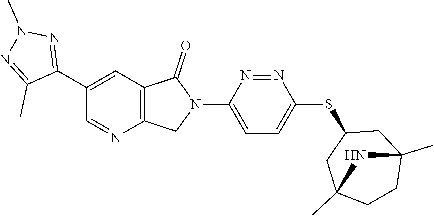 [SMILES: Cc1nn(C)nc1-c1cnc2c(c1)C(=O)N(c1ccc(S[C@H]3C[C@]4(C)CC[C@](C)(C3)N4)nn1)C2] | 3-(2,5-dimethyl-2H-1,2,3-triazol- 4-yl)-6-(6-(((1R,3s,5S)-1,5- dimethyl-8- azabicyclo[3.2.1]octan-3- yl)thio)pyridazin-3-yl)-6,7- dihydro-5H-pyrrolo[3,4-b]pyridin- 5-one |
| 612 | 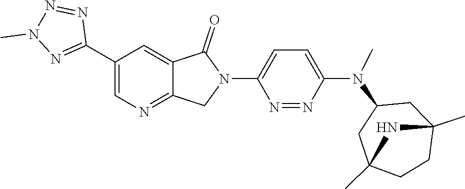 | 6-(6-(((1R,3s,5S)-1,5-dimethyl-8- azabicyclo[3.2.1]octan-3- yl)(methyl)amino)pyridazin-3-yl)- 3-(2-methyl-2H-tetrazol-5-yl)-6,7- dihydro-5H-pyrrolo[3,4-b]pyridin- 5-one |
| 622 | 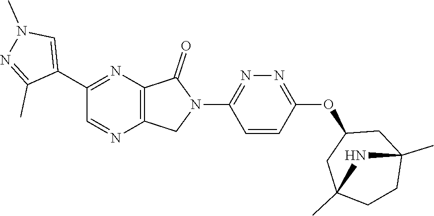 [SMILES: Cc1nn(C)cc1-c1cnc2c(n1)C(=O)N(c1ccc(O[C@H]3C[C@]4(C)CC[C@](C)(C3)N4)nn1)C2] | 3-(1,3-dimethyl-1H-pyrazol-4-yl)- 6-(6-(((1R,3s,5S)-1,5-dimethyl-8- azabicyclo[3.2.1]octan-3- yl)oxy)pyridazin-3-yl)-6,7- dihydro-5H-pyrrolo[3,4-b]pyrazin- 5-one |
| 623 | 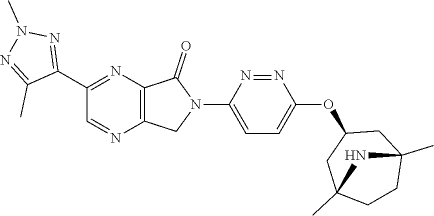 [SMILES: Cc1nn(C)nc1-c1cnc2c(n1)C(=O)N(c1ccc(O[C@H]3C[C@]4(C)CC[C@](C)(C3)N4)nn1)C2] | 3-(2,5-dimethyl-2H-1,2,3-triazol- 4-yl)-6-(6-(((1R,3s,5S)-1,5- dimethyl-8- azabicyclo[3.2.1]octan-3- yl)oxy)pyridazin-3-yl)-6,7- dihydro-5H-pyrrolo[3,4-b]pyrazin- 5-one |
| 625 | 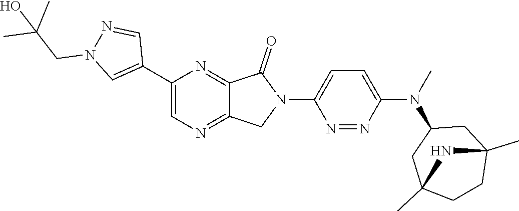 [SMILES: CN(c1ccc(N2Cc3ncc(-c4cnn(CC(C)(C)O)c4)nc3C2=O)nn1)[C@H]1C[C@]2(C)CC[C@](C)(C1)N2] | 6-(6-(((1R,3s,5S)-1,5-dimethyl-8- azabicyclo[3.2.1]octan-3- yl)(methyl)amino)pyridazin-3-yl)- 3-(1-(2-hydroxy-2-methylpropyl)- 1H-pyrazol-4-yl)-6,7-dihydro-5H- pyrrolo[3,4-b]pyrazin-5-one |
| 627 | 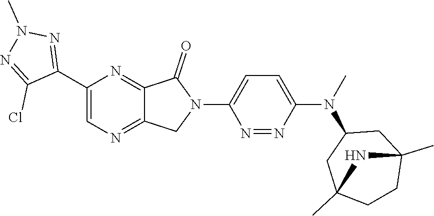 [SMILES: CN(c1ccc(N2Cc3ncc(-c4nn(C)nc4Cl)nc3C2=O)nn1)[C@H]1C[C@]2(C)CC[C@](C)(C1)N2] | 3-(5-chloro-2-methyl-2H-1,2,3- triazol-4-yl)-6-(6-(((1R,3s,5S)-1,5- dimethyl-8- azabicyclo[3.2.1]octan-3- yl)(methyl)amino)pyridazin-3-yl)- 6,7-dihydro-5H-pyrrolo[3,4- b]pyrazin-5-one |
| 631 | 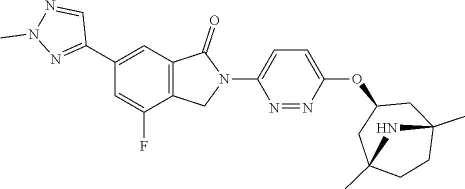 | 2-(6-(((1R,3s,5S)-1,5-dimethyl-8- azabicyclo[3.2.1]octan-3- yl)oxy)pyridazin-3-yl)-4-fluoro-6- (2-methyl-2H-1,2,3-triazol-4- yl)isoindolin-1-one |
| 632 | 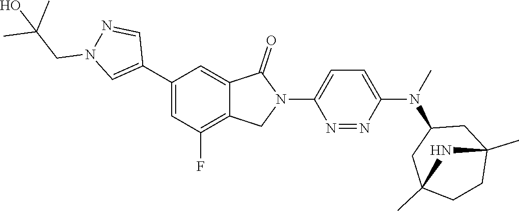 [SMILES: CN(c1ccc(N2Cc3c(F)cc(-c4cnn(CC(C)(C)O)c4)cc3C2=O)nn1)[C@H]1C[C@]2(C)CC[C@](C)(C1)N2] | 2-(6-(((1R,3s,5S)-1,5-dimethyl-8- azabicyclo[3.2.1]octan-3- yl)(methyl)amino)pyridazin-3-yl)- 4-fluoro-6-(1-(2-hydroxy-2- methylpropyl)-1H-pyrazol-4- yl)isoindolin-1-one |
| 634 | 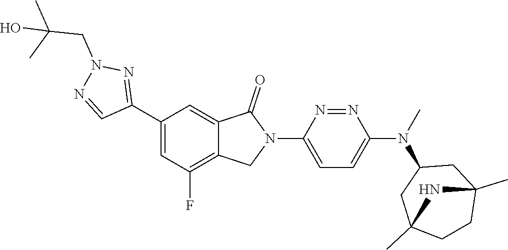 [SMILES: CN(c1ccc(N2Cc3c(F)cc(-c4cnn(CC(C)(C)O)n4)cc3C2=O)nn1)[C@H]1C[C@]2(C)CC[C@](C)(C1)N2] | 2-(6-(((1R,3s,5S)-1,5-dimethyl-8- azabicyclo[3.2.1]octan-3- yl)(methyl)amino)pyridazin-3-yl)- 4-fluoro-6-(2-(2-hydroxy-2- methylpropyl)-2H-1,2,3-triazol-4- yl)isoindolin-1-one |
| 660 | 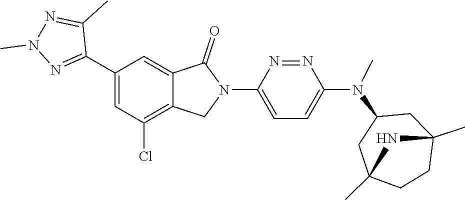 | 4-chloro-6-(2,5-dimethyl-2H- 1,2,3-triazol-4-yl)-2-(6- (((1R,3s,5S)-1,5-dimethyl-8- azabicyclo[3.2.1]octan-3- yl)(methyl)amino)pyridazin-3- yl)isoindolin-1-one |
| 661 | 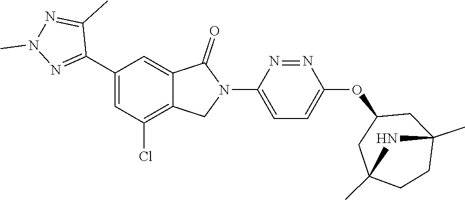 | 4-chloro-6-(2,5-dimethyl-2H- 1,2,3-triazol-4-yl)-2-(6- (((1R,3s,5S)-1,5-dimethyl-8- azabicyclo[3.2.1]octan-3- yl)oxy)pyridazin-3-yl)isoindolin- 1-one |
| 673 | 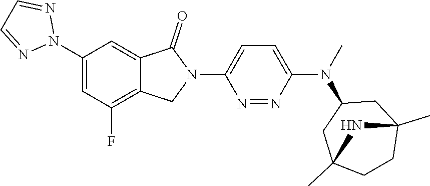 [SMILES: CN(c1ccc(N2Cc3c(F)cc(-n4nccn4)cc3C2=O)nn1)[C@H]1C[C@]2(C)CC[C@](C)(C1)N2] | 2-(6-(((1R,3s,5S)-1,5-dimethyl-8- azabicyclo[3.2.1]octan-3- yl)(methyl)amino)pyridazin-3-yl)- 4-fluoro-6-(2H-1,2,3-triazol-2- yl)isoindolin-1-one |
| 676 | 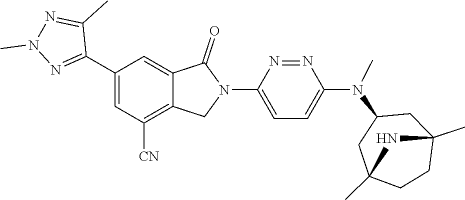 | 6-(2,5-dimethyl-2H-1,2,3-triazol- 4-yl)-2-(6-(((1R,3s,5S)-1,5- dimethyl-8- azabicyclo[3.2.1]octan-3- yl)(methyl)amino)pyridazin-3-yl)- 1-oxoisoindoline-4-carbonitrile |
| 679 | 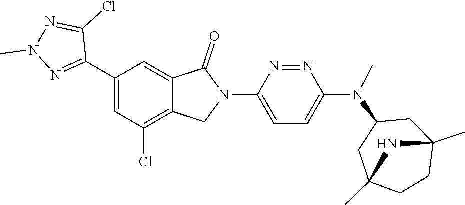 | 4-chloro-6-(5-chloro-2-methyl-2H- 1,2,3-triazol-4-yl)-2-(6- (((1R,3s,5S)-1,5-dimethyl-8- azabicyclo[3.2.1]octan-3- yl)(methyl)amino)pyridazin-3- yl)isoindolin-1-one |
| 681 | 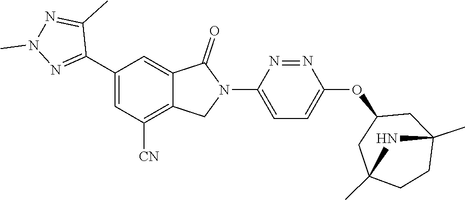 | 6-(2,5-dimethyl-2H-1,2,3-triazol- 4-yl)-2-(6-(((1R,3s,5S)-1,5- dimethyl-8- azabicyclo[3.2.1]octan-3- yl)oxy)pyridazin-3-yl)-1- oxoisoindoline-4-carbonitrile |
| 684 | 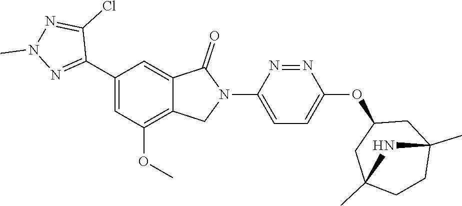 | 6-(5-chloro-2-methyl-2H-1,2,3- triazol-4-yl)-2-(6-(((1R,3s,5S)-1,5- dimethyl-8- azabicyclo[3.2.1]octan-3- yl)oxy)pyridazin-3-yl)-4- methoxyisoindolin-1-one |
| 685 | 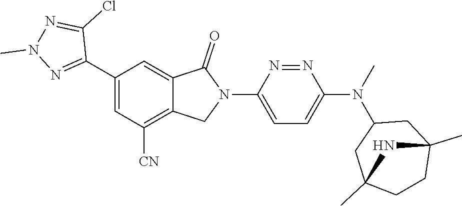 | 6-(5-chloro-2-methyl-2H-1,2,3- triazol-4-yl)-2-(6-(((1R,5S)-1,5- dimethyl-8- azabicyclo[3.2.1]octan-3- yl)(methyl)amino)pyridazin-3-yl)- 1-oxoisoindoline-4-carbonitrile |
| 686 | 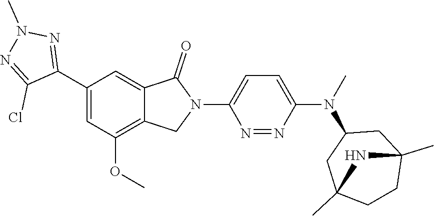 [SMILES: COc1cc(-c2nn(C)nc2Cl)cc2c1CN(c1ccc(N(C)[C@H]3C[C@]4(C)CC[C@](C)(C3)N4)nn1)C2=O] | 6-(5-chloro-2-methyl-2H-1,2,3- triazol-4-yl)-2-(6-(((1R,3s,5S)-1,5- dimethyl-8- azabicyclo[3.2.1]octan-3- yl)(methyl)amino)pyridazin-3-yl)- 4-methoxyisoindolin-1-one |
| 687 | 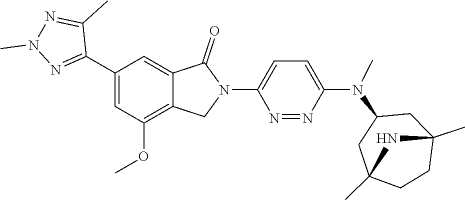 | 6-(2,5-dimethyl-2H-1,2,3-triazol- 4-yl)-2-(6-(((1R,3s,5S)-1,5- dimethyl-8- azabicyclo[3.2.1]octan-3- yl)(methyl)amino)pyridazin-3-yl)- 4-methoxyisoindolin-1-one |
| 689 | 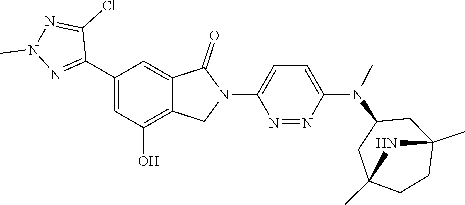 | 6-(5-chloro-2-methyl-2H-1,2,3- triazol-4-yl)-2-(6-(((1R,3s,5S)-1,5- dimethyl-8- azabicyclo[3.2.1]octan-3- yl)(methyl)amino)pyridazin-3-yl)- 4-hydroxyisoindolin-1-one |
| 693 | 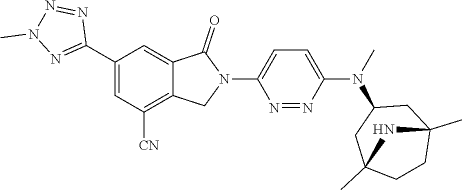 | 2-(6-(((1R,3s,5S)-1,5-dimethyl-8- azabicyclo[3.2.1]octan-3- yl)(methyl)amino)pyridazin-3-yl)- 6-(2-methyl-2H-tetrazol-5-yl)-1- oxoisoindoline-4-carbonitrile |
| 694 | 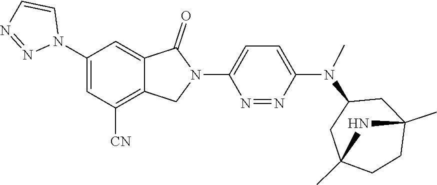 [SMILES: CN(c1ccc(N2Cc3c(C#N)cc(-n4ccnn4)cc3C2=O)nn1)[C@H]1C[C@]2(C)CC[C@](C)(C1)N2] | 3-(5-chloro-2-methyl-2H-1,2,3- triazol-4-yl)-6-(6-((6,6-dimethyl-5- azaspiro[3.5]nonan-8- yl)oxy)pyridazin-3-yl)-6,7- dihydro-5H-pyrrolo[3,4-b]pyridin- 5-one |
| 695 | 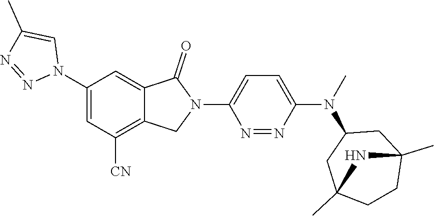 [SMILES: Cc1cn(-c2cc(C#N)c3c(c2)C(=O)N(c2ccc(N(C)[C@H]4C[C@]5(C)CC[C@](C)(C4)N5)nn2)C3)nn1] | 2-(6-(((1R,3s,5S)-1,5-dimethyl-8- azabicyclo[3.2.1]octan-3- yl)(methyl)amino)pyridazin-3-yl)- 6-(4-methyl-1H-1,2,3-triazol-1- yl)-1-oxoisoindoline-4-carbonitrile |

TABLE 3C

| No. | Chemical Structure | Name |
|---|---|---|
| 4 | 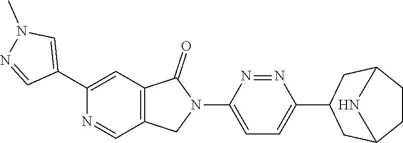 [SMILES: Cn1cc(-c2cc3c(cn2)CN(c2ccc(C4CC5CCC(C4)N5)nn2)C3=O)cn1] | 2-(6-(8-azabicyclo[3.2.1]octan-3- yl)pyridazin-3-yl)-6-(1-methyl- 1H-pyrazol-4-yl)-2,3-dihydro-1H- pyrrolo[3,4-c]pyridin-1-one |
| 5 | 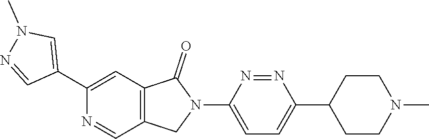 [SMILES: CN1CCC(c2ccc(N3Cc4cnc(-c5cnn(C)c5)cc4C3=O)nn2)CC1] | 6-(1-methyl-1H-pyrazol-4-yl)-2- (6-(1-methylpiperidin-4- yl)pyridazin-3-yl)-2,3-dihydro-1H- pyrrolo[3,4-c]pyridin-1-one |
| 11 | 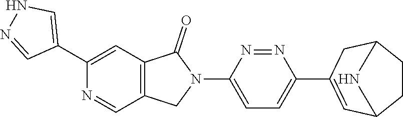 [SMILES: O=C1c2cc(-c3cn[nH]c3)ncc2CN1c1ccc(C2=CC3CCC(C2)N3)nn1] | 2-(6-(8-azabicyclo[3.2.1]oct-2-en- 3-yl)pyridazin-3-yl)-6-(1H- pyrazol-4-yl)-2,3-dihydro-1H- pyrrolo[3,4-c]pyridin-1-one |
| 12 | 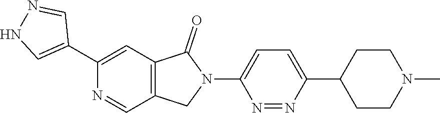 [SMILES: CN1CCC(c2ccc(N3Cc4cnc(-c5cn[nH]c5)cc4C3=O)nn2)CC1] | 2-(6-(1-methylpiperidin-4- yl)pyridazin-3-yl)-6-(1H-pyrazol- 4-yl)-2,3-dihydro-1H-pyrrolo[3,4- c]pyridin-1-one |
| 13 | 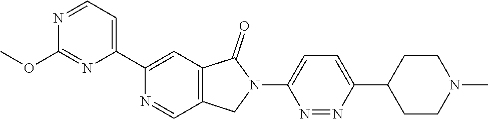 | 6-(2-methoxypyrimidin-4-yl)-2-(6- (1-methylpiperidin-4-yl)pyridazin- 3-yl)-2,3-dihydro-1H-pyrrolo[3,4- c]pyridin-1-one |
| 14 | 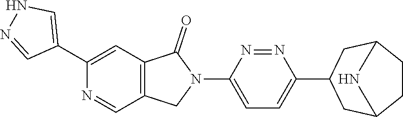 [SMILES: O=C1c2cc(-c3cn[nH]c3)ncc2CN1c1ccc(C2CC3CCC(C2)N3)nn1] | 2-(6-(8-azabicyclo[3.2.1]octan-3- yl)pyridazin-3-yl)-6-(1H-pyrazol- 4-yl)-2,3-dihydro-1H-pyrrolo[3,4- c]pyridin-1-one |
| 16 | 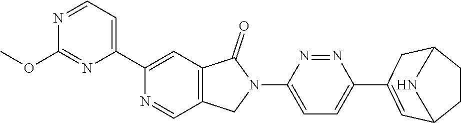 [SMILES: COc1nccc(-c2cc3c(cn2)CN(c2ccc(C4=CC5CCC(C4)N5)nn2)C3=O)n1] | 2-(6-(8-azabicyclo[3.2.1]oct-2-en- 3-yl)pyridazin-3-yl)-6-(2- methoxypyrimidin-4-yl)-2,3- dihydro-1H-pyrrolo[3,4-c]pyridin- 1-one |
| 22 | 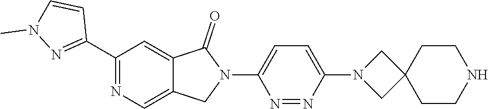 [SMILES: Cn1ccc(-c2cc3c(cn2)CN(c2ccc(N4CC5(CCNCC5)C4)nn2)C3=O)n1] | 2-(6-(2,7-diazaspiro[3.5]nonan-2- yl)pyridazin-3-yl)-6-(1-methyl- 1H-pyrazol-3-yl)-2,3-dihydro-1H- pyrrolo[3,4-c]pyridin-1-one |
| 25 | 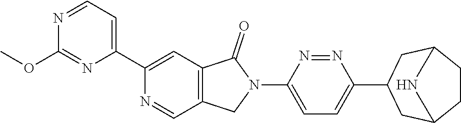 [SMILES: COc1nccc(-c2cc3c(cn2)CN(c2ccc(C4CC5CCC(C4)N5)nn2)C3=O)n1] | 2-(6-(8-azabicyclo[3.2.1]octan-3- yl)pyridazin-3-yl)-6-(2- methoxypyrimidin-4-yl)-2,3- dihydro-1H-pyrrolo[3,4-c]pyridin- 1-one |
| 32 | 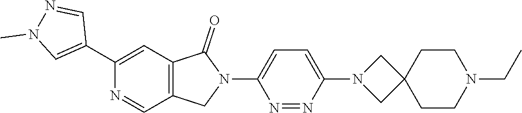 [SMILES: CCN1CCC2(CC1)CN(c1ccc(N3Cc4cnc(-c5cnn(C)c5)cc4C3=O)nn1)C2] | 2-(6-(7-ethyl-2,7- diazaspiro[3.5]nonan-2- yl)pyridazin-3-yl)-6-(1-methyl- 1H-pyrazol-4-yl)-2,3-dihydro-1H- pyrrolo[3,4-c]pyridin-1-one |
| 33 | 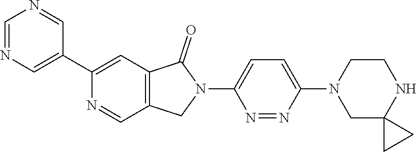 [SMILES: O=C1c2cc(-c3cncnc3)ncc2CN1c1ccc(N2CCNC3(CC3)C2)nn1] | 2-(6-(4,7-diazaspiro[2.5]octan-7- yl)pyridazin-3-yl)-6-(pyrimidin-5- yl)-2,3-dihydro-1H-pyrrolo[3,4- c]pyridin-1-one |
| 34 | 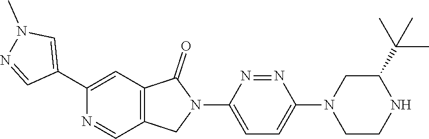 [SMILES: Cn1cc(-c2cc3c(cn2)CN(c2ccc(N4CCN[C@@H](C(C)(C)C)C4)nn2)C3=O)cn1] | (S)-2-(6-(3-(tert-butyl)piperazin-1- yl)pyridazin-3-yl)-6-(1-methyl- 1H-pyrazol-4-yl)-2,3-dihydro-1H- pyrrolo[3,4-c]pyridin-1-one |
| 36 | 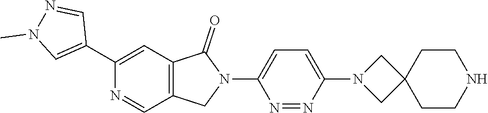 [SMILES: Cn1cc(-c2cc3c(cn2)CN(c2ccc(N4CC5(CCNCC5)C4)nn2)C3=O)cn1] | 2-(6-(2,7-diazaspiro[3.5]nonan-2- yl)pyridazin-3-yl)-6-(1-methyl- 1H-pyrazol-4-yl)-2,3-dihydro-1H- pyrrolo[3,4-c]pyridin-1-one |
| 37 | 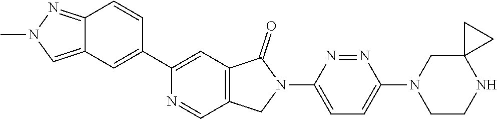 [SMILES: Cn1cc2cc(-c3cc4c(cn3)CN(c3ccc(N5CCNC6(CC6)C5)nn3)C4=O)ccc2n1] | 2-(6-(4,7-diazaspiro[2.5]octan-7- yl)pyridazin-3-yl)-6-(2-methyl- 2H-indazol-5-yl)-2,3-dihydro-1H- pyrrolo[3,4-c]pyridin-1-one |
| 38 | 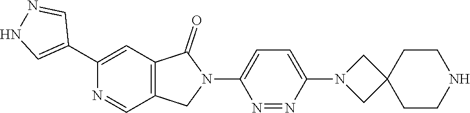 [SMILES: O=C1c2cc(-c3cn[nH]c3)ncc2CN1c1ccc(N2CC3(CCNCC3)C2)nn1] | 2-(6-(2,7-diazaspiro[3.5]nonan-2- yl)pyridazin-3-yl)-6-(1H-pyrazol- 4-yl)-2,3-dihydro-1H-pyrrolo[3,4- c]pyridin-1-one |
| 39 | 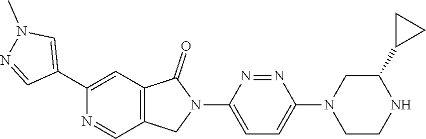 [SMILES: Cn1cc(-c2cc3c(cn2)CN(c2ccc(N4CCN[C@@H](C5CC5)C4)nn2)C3=O)cn1] | (S)-2-(6-(3-cyclopropylpiperazin- 1-yl)pyridazin-3-yl)-6-(1-methyl- 1H-pyrazol-4-yl)-2,3-dihydro-1H- pyrrolo[3,4-c]pyridin-1-one |
| 40 | 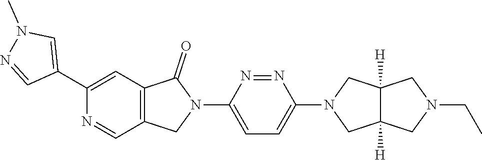 [SMILES: CCN1C[C@@H]2CN(c3ccc(N4Cc5cnc(-c6cnn(C)c6)cc5C4=O)nn3)C[C@@H]2C1] | 2-(6-((3aR,6aS)-5- ethylhexahydropyrrolo[3,4- c]pyrrol-2(1H)-yl)pyridazin-3-yl)- 6-(1-methyl-1H-pyrazol-4-yl)-2,3- dihydro-1H-pyrrolo[3,4-c]pyridin- 1-one |
| 41 | 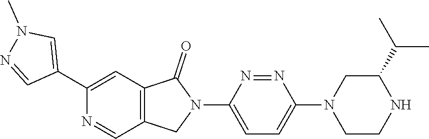 [SMILES: CC(C)[C@H]1CN(c2ccc(N3Cc4cnc(-c5cnn(C)c5)cc4C3=O)nn2)CCN1] | (S)-2-(6-(3-isopropylpiperazin-1- yl)pyridazin-3-yl)-6-(1-methyl- 1H-pyrazol-4-yl)-2,3-dihydro-1H- pyrrolo[3,4-c]pyridin-1-one |
| 44 | 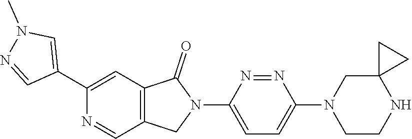 [SMILES: Cn1cc(-c2cc3c(cn2)CN(c2ccc(N4CCNC5(CC5)C4)nn2)C3=O)cn1] | 2-(6-(4,7-diazaspiro[2.5]octan-7- yl)pyridazin-3-yl)-6-(1-methyl- 1H-pyrazol-4-yl)-2,3-dihydro-1H- pyrrolo[3,4-c]pyridin-1-one |
| 45 | 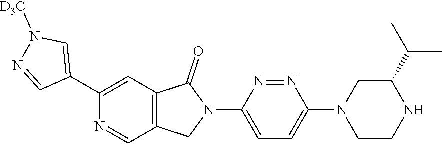 [SMILES: [2H]C([2H])([2H])n1cc(-c2cc3c(cn2)CN(c2ccc(N4CCN[C@@H](C(C)C)C4)nn2)C3=O)cn1] | (S)-2-(6-(3-isopropylpiperidin-1- yl)pyridazin-3-yl)-6-(1-(methyl- d3)-1H-pyrazol-4-yl)-2,3-dihydro- 1H-pyrrolo[3,4-c]pyridin-1-one |
| 46 | 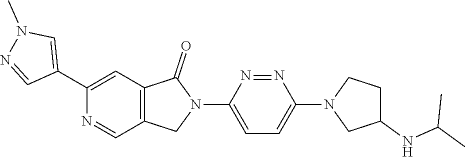 [SMILES: CC(C)NC1CCN(c2ccc(N3Cc4cnc(-c5cnn(C)c5)cc4C3=O)nn2)C1] | 2-(6-(3- (isopropylamino)pyrrolidin-1- yl)pyridazin-3-yl)-6-(1-methyl- 1H-pyrazol-4-yl)-2,3-dihydro-1H- pyrrolo[3,4-c]pyridin-1-one |
| 48 | 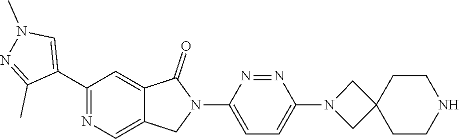 [SMILES: Cc1nn(C)cc1-c1cc2c(cn1)CN(c1ccc(N3CC4(CCNCC4)C3)nn1)C2=O] | 2-(6-(2,7-diazaspiro[3.5]nonan-2- yl)pyridazin-3-yl)-6-(1,3-dimethyl- 1H-pyrazol-4-yl)-2,3-dihydro-1H- pyrrolo[3,4-c]pyridin-1-one |
| 50 | 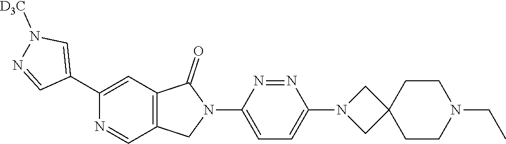 [SMILES: [2H]C([2H])([2H])n1cc(-c2cc3c(cn2)CN(c2ccc(N4CC5(CCN(CC)CC5)C4)nn2)C3=O)cn1] | 2-(6-(7-ethyl-2,7- diazaspiro[3.5]nonan-2- yl)pyridazin-3-yl)-6-(1-(methyl- d3)-1H-pyrazol-4-yl)-2,3-dihydro- 1H-pyrrolo[3,4-c]pyridin-1-one |
| 52 | 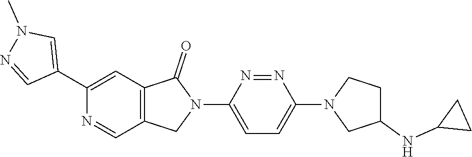 [SMILES: Cn1cc(-c2cc3c(cn2)CN(c2ccc(N4CCC(NC5CC5)C4)nn2)C3=O)cn1] | 2-(6-(3- (cyclopropylamino)pyrrolidin-1- yl)pyridazin-3-yl)-6-(1-methyl- 1H-pyrazol-4-yl)-2,3-dihydro-1H- pyrrolo[3,4-c]pyridin-1-one |
| 53 | 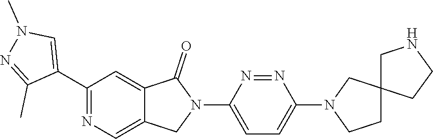 [SMILES: Cc1nn(C)cc1-c1cc2c(cn1)CN(c1ccc(N3CCC4(CCNC4)C3)nn1)C2=O] | 2-(6-(2,7-diazaspiro[4.4]nonan-2- yl)pyridazin-3-yl)-6-(1,3-dimethyl- 1H-pyrazol-4-yl)-2,3-dihydro-1H- pyrrolo[3,4-c]pyridin-1-one |
| 95 | 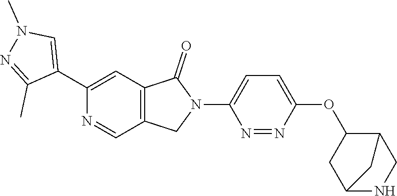 [SMILES: Cc1nn(C)cc1-c1cc2c(cn1)CN(c1ccc(OC3CC4CC3CN4)nn1)C2=O] | 2-(6-((2-azabicyclo[2.2.1]heptan- 5-yl)oxy)pyridazin-3-yl)-6-(1,3- dimethyl-1H-pyrazol-4-yl)-2,3- dihydro-1H-pyrrolo[3,4-c]pyridin- 1-one |
| 120 | 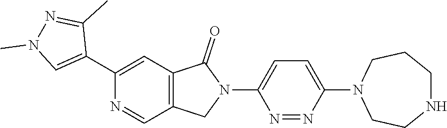 [SMILES: Cc1nn(C)cc1-c1cc2c(cn1)CN(c1ccc(N3CCCNCC3)nn1)C2=O] | 2-(6-(1,4-diazepan-1-yl)pyridazin- 3-yl)-6-(1,3-dimethyl-1H-pyrazol- 4-yl)-2,3-dihydro-1H-pyrrolo[3,4- c]pyridin-1-one |
| 121 | 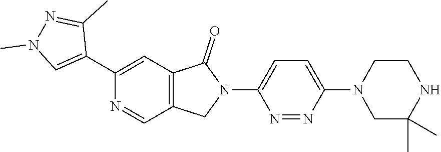 | 6-(1,3-dimethyl-1H-pyrazol-4-yl)- 2-(6-(3,3-dimethylpiperazin-1- yl)pyridazin-3-yl)-2,3-dihydro-1H- pyrrolo[3,4-c]pyridin-1-one |
| 122 | 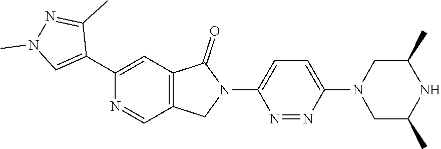 | 6-(1,3-dimethyl-1H-pyrazol-4-yl)- 2-(6-((3R,5S)-3,5- dimethylpiperazin-1-yl)pyridazin- 3-yl)-2,3-dihydro-1H-pyrrolo[3,4- c]pyridin-1-one |
| 123 | 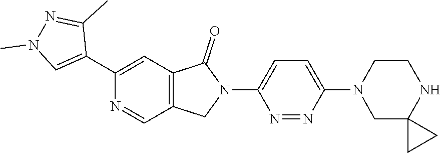 [SMILES: Cc1nn(C)cc1-c1cc2c(cn1)CN(c1ccc(N3CCNC4(CC4)C3)nn1)C2=O] | 2-(6-(4,7-diazaspiro[2.5]octan-7- yl)pyridazin-3-yl)-6-(1,3-dimethyl- 1H-pyrazol-4-yl)-2,3-dihydro-1H- pyrrolo[3,4-c]pyridin-1-one |
| 124 | 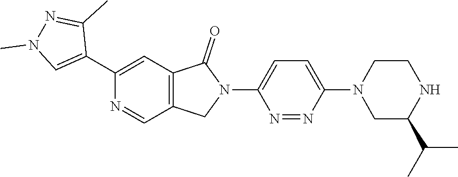 [SMILES: Cc1nn(C)cc1-c1cc2c(cn1)CN(c1ccc(N3CCN[C@@H](C(C)C)C3)nn1)C2=O] | (S)-6-(1,3-dimethyl-1H-pyrazol-4- yl)-2-(6-(3-isopropylpiperazin-1- yl)pyridazin-3-yl)-2,3-dihydro-1H- pyrrolo[3,4-c]pyridin-1-one |
| 125 | 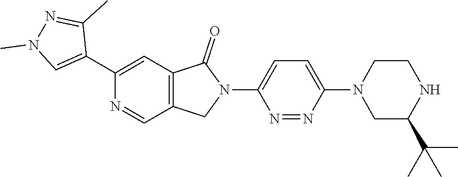 [SMILES: Cc1nn(C)cc1-c1cc2c(cn1)CN(c1ccc(N3CCN[C@@H](C(C)(C)C)C3)nn1)C2=O] | (S)-2-(6-(3-(tert-butyl)piperazin-1- yl)pyridazin-3-yl)-6-(1,3-dimethyl- 1H-pyrazol-4-yl)-2,3-dihydro-1H- pyrrolo[3,4-c]pyridin-1-one |
| 126 | 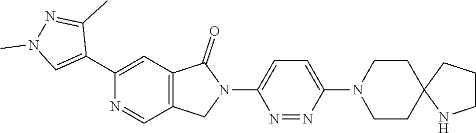 [SMILES: Cc1nn(C)cc1-c1cc2c(cn1)CN(c1ccc(N3CCC4(CCCN4)CC3)nn1)C2=O] | 2-(6-(1,8-diazaspiro[4.5]decan-8- yl)pyridazin-3-yl)-6-(1,3-dimethyl- 1H-pyrazol-4-yl)-2,3-dihydro-1H- pyrrolo[3,4-c]pyridin-1-one |
| 127 | 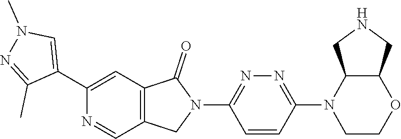 [SMILES: Cc1nn(C)cc1-c1cc2c(cn1)CN(c1ccc(N3CCO[C@@H]4CNC[C@@H]43)nn1)C2=O] | 6-(1,3-dimethyl-1H-pyrazol-4-yl)- 2-(6-((4aS,7aR)- hexahydropyrrolo[3,4- b][1,4]oxazin-4(4aH)-yl)pyridazin- 3-yl)-2,3-dihydro-1H-pyrrolo[3,4- c]pyridin-1-one |
| 128 | 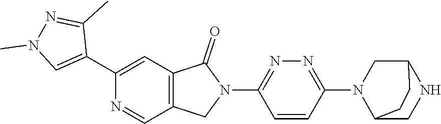 [SMILES: Cc1nn(C)cc1-c1cc2c(cn1)CN(c1ccc(N3CC4CCC3CN4)nn1)C2=O] | 2-(6-(2,5-diazabicyclo[2.2.2]octan- 2-yl)pyridazin-3-yl)-6-(1,3- dimethyl-1H-pyrazol-4-yl)-2,3- dihydro-1H-pyrrolo[3,4-c]pyridin- 1-one |
| 129 | 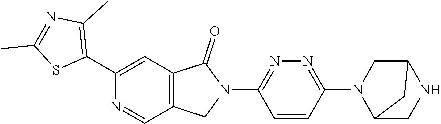 [SMILES: Cc1nc(C)c(-c2cc3c(cn2)CN(c2ccc(N4CC5CC4CN5)nn2)C3=O)s1] | 2-(6-(2,5- diazabicyclo[2.2.1]heptan-2- yl)pyridazin-3-yl)-6-(2,4- dimethylthiazol-5-yl)-2,3-dihydro- 1H-pyrrolo[3,4-c]pyridin-1-one |
| 130 | 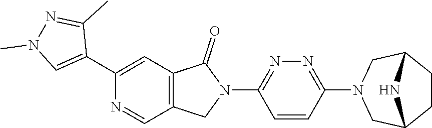 [SMILES: Cc1nn(C)cc1-c1cc2c(cn1)CN(c1ccc(N3C[C@H]4CC[C@@H](C3)N4)nn1)C2=O] | 2-(6-((1R,5S)-3,8- diazabicyclo[3.2.1]octan-3- yl)pyridazin-3-yl)-6-(1,3-dimethyl- 1H-pyrazol-4-yl)-2,3-dihydro-1H- pyrrolo[3,4-c]pyridin-1-one |
| 131 | 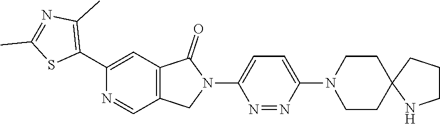 [SMILES: Cc1nc(C)c(-c2cc3c(cn2)CN(c2ccc(N4CCC5(CCCN5)CC4)nn2)C3=O)s1] | 2-(6-(1,8-diazaspiro[4.5]decan-8- yl)pyridazin-3-yl)-6-(2,4- dimethylthiazol-5-yl)-2,3-dihydro- 1H-pyrrolo[3,4-c]pyridin-1-one |
| 132 | 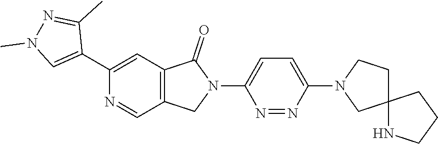 [SMILES: Cc1nn(C)cc1-c1cc2c(cn1)CN(c1ccc(N3CCC4(CCCN4)C3)nn1)C2=O] | 2-(6-(1,7-diazaspiro[4.4]nonan-7- yl)pyridazin-3-yl)-6-(1,3-dimethyl- 1H-pyrazol-4-yl)-2,3-dihydro-1H- pyrrolo[3,4-c]pyridin-1-one |
| 133 | 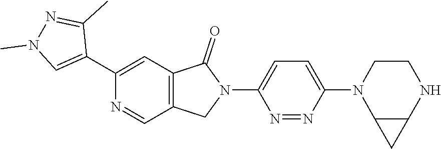 [SMILES: Cc1nn(C)cc1-c1cc2c(cn1)CN(c1ccc(N3CCNC4CC43)nn1)C2=O] | 2-(6-(2,5- diazabicyclo[4.1.0]heptan-2- yl)pyridazin-3-yl)-6-(1,3-dimethyl- 1H-pyrazol-4-yl)-2,3-dihydro-1H- pyrrolo[3,4-c]pyridin-1-one |
| 136 | 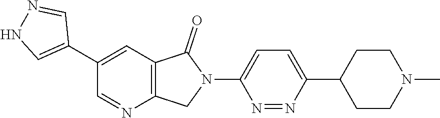 [SMILES: CN1CCC(c2ccc(N3Cc4ncc(-c5cn[nH]c5)cc4C3=O)nn2)CC1] | 6-(6-(1-methylpiperidin-4- yl)pyridazin-3-yl)-3-(1H-pyrazol- 4-yl)-6,7-dihydro-5H-pyrrolo[3,4- b]pyridin-5-one |
| 137 | 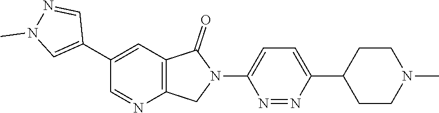 | 3-(1-methyl-1H-pyrazol-4-yl)-6- (6-(1-methylpiperidin-4- yl)pyridazin-3-yl)-6,7-dihydro-5H- pyrrolo[3,4-b]pyridin-5-one |
| 145 | 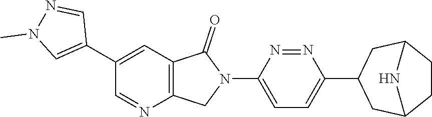 [SMILES: Cn1cc(-c2cnc3c(c2)C(=O)N(c2ccc(C4CC5CCC(C4)N5)nn2)C3)cn1] | 6-(6-(8-azabicyclo[3.2.1]octan-3- yl)pyridazin-3-yl)-3-(1-methyl- 1H-pyrazol-4-yl)-6,7-dihydro-5H- pyrrolo[3,4-b]pyridin-5-one |
| 146 | 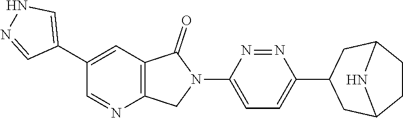 [SMILES: O=C1c2cc(-c3cn[nH]c3)cnc2CN1c1ccc(C2CC3CCC(C2)N3)nn1] | 6-(6-(8-azabicyclo[3.2.1]octan-3- yl)pyridazin-3-yl)-3-(1H-pyrazol- 4-yl)-6,7-dihydro-5H-pyrrolo[3,4- b]pyridin-5-one |
| 147 | 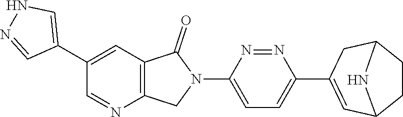 [SMILES: O=C1c2cc(-c3cn[nH]c3)cnc2CN1c1ccc(C2=CC3CCC(C2)N3)nn1] | 6-(6-(8-azabicyclo[3.2.1]oct-2-en- 3-yl)pyridazin-3-yl)-3-(1H- pyrazol-4-yl)-6,7-dihydro-5H- pyrrolo[3,4-b]pyridin-5-one |
| 151 | 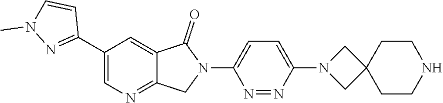 [SMILES: Cn1ccc(-c2cnc3c(c2)C(=O)N(c2ccc(N4CC5(CCNCC5)C4)nn2)C3)n1] | 6-(6-(2,7-diazaspiro[3.5]nonan-2- yl)pyridazin-3-yl)-3-(1-methyl- 1H-pyrazol-3-yl)-6,7-dihydro-5H- pyrrolo[3,4-b]pyridin-5-one |
| 152 | 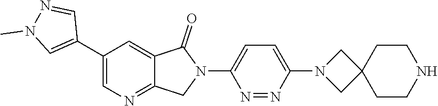 [SMILES: Cn1cc(-c2cnc3c(c2)C(=O)N(c2ccc(N4CC5(CCNCC5)C4)nn2)C3)cn1] | 6-(6-(2,7-diazaspiro[3.5]nonan-2- yl)pyridazin-3-yl)-3-(1-methyl- 1H-pyrazol-4-yl)-6,7-dihydro-5H- pyrrolo[3,4-b]pyridin-5-one |
| 153 | 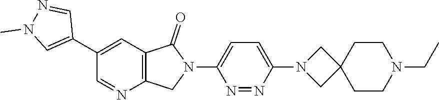 [SMILES: CCN1CCC2(CC1)CN(c1ccc(N3Cc4ncc(-c5cnn(C)c5)cc4C3=O)nn1)C2] | 6-(6-(7-ethyl-2,7- diazaspiro[3.5]nonan-2- yl)pyridazin-3-yl)-3-(1-methyl- 1H-pyrazol-4-yl)-6,7-dihydro-5H- pyrrolo[3,4-b]pyridin-5-one |
| 154 | 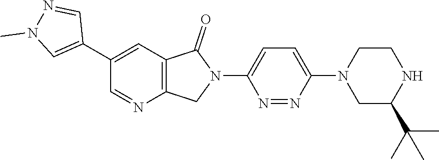 [SMILES: Cn1cc(-c2cnc3c(c2)C(=O)N(c2ccc(N4CCN[C@@H](C(C)(C)C)C4)nn2)C3)cn1] | (S)-6-(6-(3-(tert-butyl)piperazin-1- yl)pyridazin-3-yl)-3-(1-methyl- 1H-pyrazol-4-yl)-6,7-dihydro-5H- pyrrolo[3,4-b]pyridin-5-one |
| 155 | 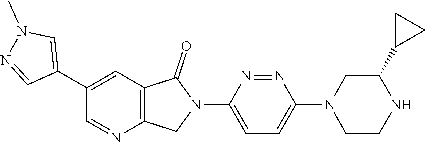 [SMILES: Cn1cc(-c2cnc3c(c2)C(=O)N(c2ccc(N4CCN[C@@H](C5CC5)C4)nn2)C3)cn1] | (S)-6-(6-(3-cyclopropylpiperazin- 1-yl)pyridazin-3-yl)-3-(1-methyl- 1H-pyrazol-4-yl)-6,7-dihydro-5H- pyrrolo[3,4-b]pyridin-5-one |
| 157 | 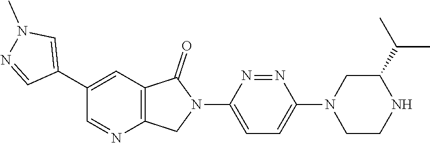 [SMILES: CC(C)[C@H]1CN(c2ccc(N3Cc4ncc(-c5cnn(C)c5)cc4C3=O)nn2)CCN1] | (S)-6-(6-(3-isopropylpiperazin-1- yl)pyridazin-3-yl)-3-(1-methyl- 1H-pyrazol-4-yl)-6,7-dihydro-5H- pyrrolo[3,4-b]pyridin-5-one |
| 158 | 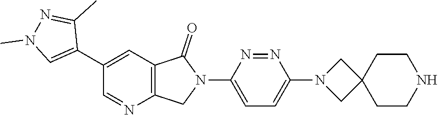 [SMILES: Cc1nn(C)cc1-c1cnc2c(c1)C(=O)N(c1ccc(N3CC4(CCNCC4)C3)nn1)C2] | 6-(6-(2,7-diazaspiro[3.5]nonan-2- yl)pyridazin-3-yl)-3-(1,3-dimethyl- 1H-pyrazol-4-yl)-6,7-dihydro-5H- pyrrolo[3,4-b]pyridin-5-one |
| 159 | 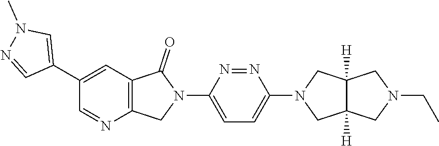 [SMILES: CCN1C[C@@H]2CN(c3ccc(N4Cc5ncc(-c6cnn(C)c6)cc5C4=O)nn3)C[C@@H]2C1] | 6-(6-((3aR,6aS)-5- ethylhexahydropyrrolo[3,4- c]pyrrol-2(1H)-yl)pyridazin-3-yl)- 3-(1-methyl-1H-pyrazol-4-yl)-6,7- dihydro-5H-pyrrolo[3,4-b]pyridin- 5-one |
| 165 | 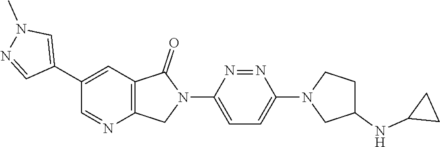 [SMILES: Cn1cc(-c2cnc3c(c2)C(=O)N(c2ccc(N4CCC(NC5CC5)C4)nn2)C3)cn1] | 6-(6-(3- (cyclopropylamino)pyrrolidin-1- yl)pyridazin-3-yl)-3-(1-methyl- 1H-pyrazol-4-yl)-6,7-dihydro-5H- pyrrolo[3,4-b]pyridin-5-one |
| 170 | 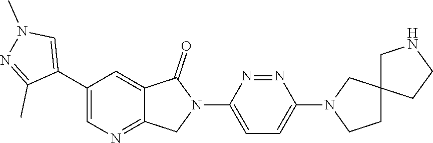 [SMILES: Cc1nn(C)cc1-c1cnc2c(c1)C(=O)N(c1ccc(N3CCC4(CCNC4)C3)nn1)C2] | 6-(6-(2,7-diazaspiro[4.4]nonan-2- yl)pyridazin-3-yl)-3-(1,3-dimethyl- 1H-pyrazol-4-yl)-6,7-dihydro-5H- pyrrolo[3,4-b]pyridin-5-one |
| 252 | 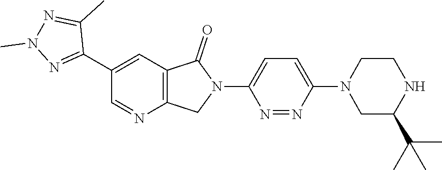 [SMILES: Cc1nn(C)nc1-c1cnc2c(c1)C(=O)N(c1ccc(N3CCN[C@@H](C(C)(C)C)C3)nn1)C2] | (S)-6-(6-(3-(tert-butyl)piperazin-1- yl)pyridazin-3-yl)-3-(2,5-dimethyl- 2H-1,2,3-triazol-4-yl)-6,7-dihydro- 5H-pyrrolo[3,4-b]pyridin-5-one |
| 254 | 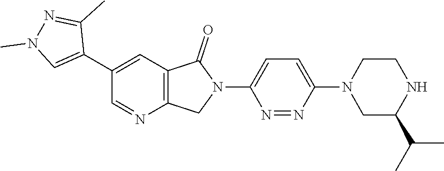 [SMILES: Cc1nn(C)cc1-c1cnc2c(c1)C(=O)N(c1ccc(N3CCN[C@@H](C(C)C)C3)nn1)C2] | (S)-3-(1,3-dimethyl-1H-pyrazol-4- yl)-6-(6-(3-isopropylpiperazin-1- yl)pyridazin-3-yl)-6,7-dihydro-5H- pyrrolo[3,4-b]pyridin-5-one |
| 255 | 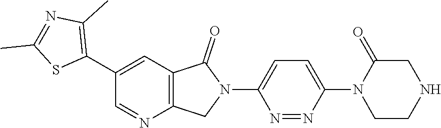 [SMILES: Cc1nc(C)c(-c2cnc3c(c2)C(=O)N(c2ccc(N4CCNCC4=O)nn2)C3)s1] | 3-(2,4-dimethylthiazol-5-yl)-6-(6- (2-oxopiperazin-1-yl)pyridazin-3- yl)-6,7-dihydro-5H-pyrrolo[3,4- b]pyridin-5-one |
| 256 | 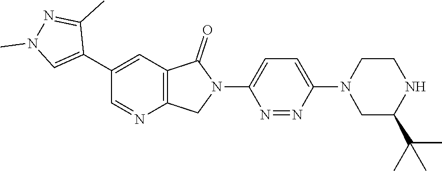 [SMILES: Cc1nn(C)cc1-c1cnc2c(c1)C(=O)N(c1ccc(N3CCN[C@@H](C(C)(C)C)C3)nn1)C2] | (S)-6-(6-(3-(tert-butyl)piperazin-1- yl)pyridazin-3-yl)-3-(1,3-dimethyl- 1H-pyrazol-4-yl)-6,7-dihydro-5H- pyrrolo[3,4-b]pyridin-5-one |
| 257 | 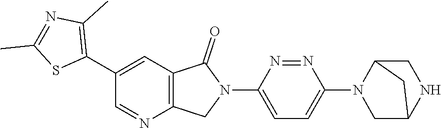 [SMILES: Cc1nc(C)c(-c2cnc3c(c2)C(=O)N(c2ccc(N4CC5CC4CN5)nn2)C3)s1] | 6-(6-(2,5- diazabicyclo[2.2.1]heptan-2- yl)pyridazin-3-yl)-3-(2,4- dimethylthiazol-5-yl)-6,7-dihydro- 5H-pyrrolo[3,4-b]pyridin-5-one |
| 258 | 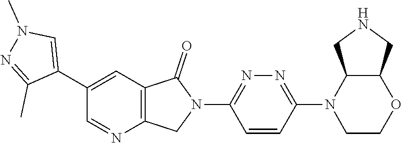 [SMILES: Cc1nn(C)cc1-c1cnc2c(c1)C(=O)N(c1ccc(N3CCO[C@@H]4CNC[C@@H]43)nn1)C2] | 3-(1,3-dimethyl-1H-pyrazol-4-yl)- 6-(6-((4aS,7aR)- hexahydropyrrolo[3,4- b][1,4]oxazin-4(4aH)-yl)pyridazin- 3-yl)-6,7-dihydro-5H-pyrrolo[3,4- b]pyridin-5-one |
| 259 | 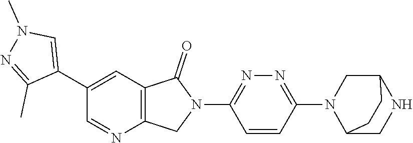 [SMILES: Cc1nn(C)cc1-c1cnc2c(c1)C(=O)N(c1ccc(N3CC4CCC3CN4)nn1)C2] | 6-(6-(2,5-diazabicyclo[2.2.2]octan- 2-yl)pyridazin-3-yl)-3-(1,3- dimethyl-1H-pyrazol-4-yl)-6,7- dihydro-5H-pyrrolo[3,4-b]pyridin- 5-one |
| 260 | 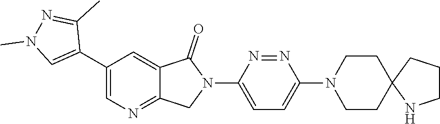 [SMILES: Cc1nn(C)cc1-c1cnc2c(c1)C(=O)N(c1ccc(N3CCC4(CCCN4)CC3)nn1)C2] | 6-(6-(1,8-diazaspiro[4.5]decan-8- yl)pyridazin-3-yl)-3-(1,3-dimethyl- 1H-pyrazol-4-yl)-6,7-dihydro-5H- pyrrolo[3,4-b]pyridin-5-one |
| 263 | 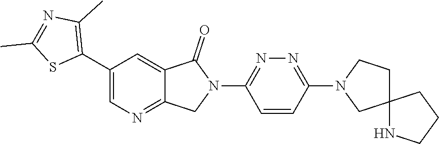 [SMILES: Cc1nc(C)c(-c2cnc3c(c2)C(=O)N(c2ccc(N4CCC5(CCCN5)C4)nn2)C3)s1] | 6-(6-(1,7-diazaspiro[4.4]nonan-7- yl)pyridazin-3-yl)-3-(2,4- dimethylthiazol-5-yl)-6,7-dihydro- 5H-pyrrolo[3,4-b]pyridin-5-one |
| 264 | 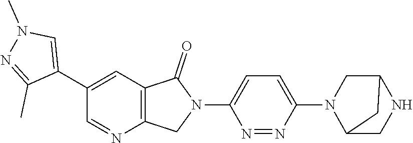 [SMILES: Cc1nn(C)cc1-c1cnc2c(c1)C(=O)N(c1ccc(N3CC4CC3CN4)nn1)C2] | 6-(6-(2,5- diazabicyclo[2.2.1]heptan-2- yl)pyridazin-3-yl)-3-(1,3-dimethyl- 1H-pyrazol-4-yl)-6,7-dihydro-5H- pyrrolo[3,4-b]pyridin-5-one |
| 265 | 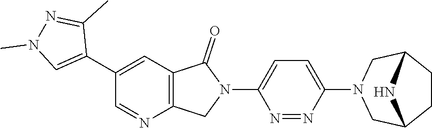 [SMILES: Cc1nn(C)cc1-c1cnc2c(c1)C(=O)N(c1ccc(N3C[C@H]4CC[C@@H](C3)N4)nn1)C2] | 6-(6-((1R,5S)-3,8- diazabicyclo[3.2.1]octan-3- yl)pyridazin-3-yl)-3-(1,3-dimethyl- 1H-pyrazol-4-yl)-6,7-dihydro-5H- pyrrolo[3,4-b]pyridin-5-one |
| 266 | 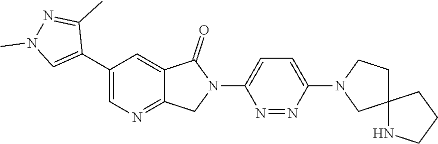 [SMILES: Cc1nn(C)cc1-c1cnc2c(c1)C(=O)N(c1ccc(N3CCC4(CCCN4)C3)nn1)C2] | 6-(6-(1,7-diazaspiro[4.4]nonan-7- yl)pyridazin-3-yl)-3-(1,3-dimethyl- 1H-pyrazol-4-yl)-6,7-dihydro-5H- pyrrolo[3,4-b]pyridin-5-one |
| 267 | 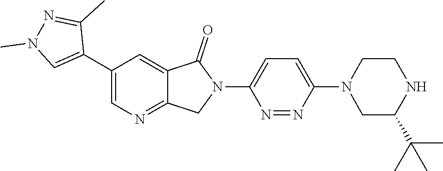 [SMILES: Cc1nn(C)cc1-c1cnc2c(c1)C(=O)N(c1ccc(N3CCN[C@H](C(C)(C)C)C3)nn1)C2] | (R)-6-(6-(3-(tert-butyl)piperazin-1- yl)pyridazin-3-yl)-3-(1,3-dimethyl- 1H-pyrazol-4-yl)-6,7-dihydro-5H- pyrrolo[3,4-b]pyridin-5-one |
| 268 | 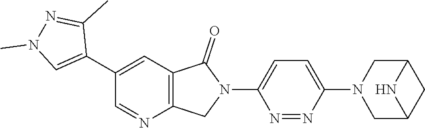 [SMILES: Cc1nn(C)cc1-c1cnc2c(c1)C(=O)N(c1ccc(N3CC4CC(C3)N4)nn1)C2] | 6-(6-(3,6- diazabicyclo[3.1.1]heptan-3- yl)pyridazin-3-yl)-3-(1,3-dimethyl- 1H-pyrazol-4-yl)-6,7-dihydro-5H- pyrrolo[3,4-b]pyridin-5-one |
| 270 | 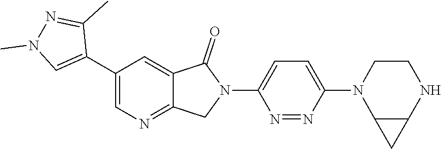 [SMILES: Cc1nn(C)cc1-c1cnc2c(c1)C(=O)N(c1ccc(N3CCNC4CC43)nn1)C2] | 6-(6-(2,5- diazabicyclo[4.1.0]heptan-2- yl)pyridazin-3-yl)-3-(1,3-dimethyl- 1H-pyrazol-4-yl)-6,7-dihydro-5H- pyrrolo[3,4-b]pyridin-5-one |
| 271 | 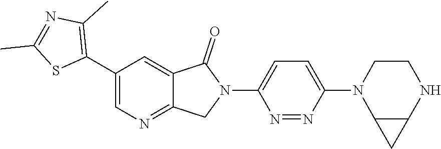 [SMILES: Cc1nc(C)c(-c2cnc3c(c2)C(=O)N(c2ccc(N4CCNC5CC54)nn2)C3)s1] | 6-(6-(2,5- diazabicyclo[4.1.0]heptan-2- yl)pyridazin-3-yl)-3-(2,4- dimethylthiazol-5-yl)-6,7-dihydro- 5H-pyrrolo[3,4-b]pyridin-5-one |
| 272 | 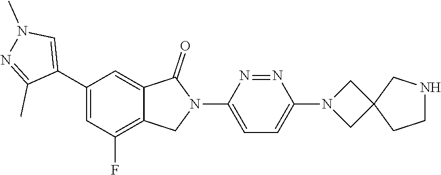 [SMILES: Cc1nn(C)cc1-c1cc(F)c2c(c1)C(=O)N(c1ccc(N3CC4(CCNC4)C3)nn1)C2] | 2-(6-(2,6-diazaspiro[3.4]octan-2- yl)pyridazin-3-yl)-6-(1,3-dimethyl- 1H-pyrazol-4-yl)-4- fluoroisoindolin-1-one |
| 273 | 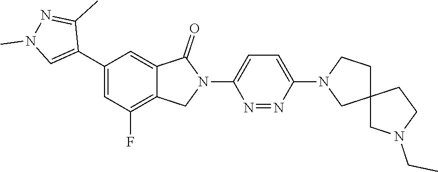 [SMILES: CCN1CCC2(CCN(c3ccc(N4Cc5c(F)cc(-c6cn(C)nc6C)cc5C4=O)nn3)C2)C1] | 6-(1,3-dimethyl-1H-pyrazol-4-yl)- 2-(6-(7-ethyl-2,7- diazaspiro[4.4]nonan-2- yl)pyridazin-3-yl)-4- fluoroisoindolin-1-one |
| 295 | 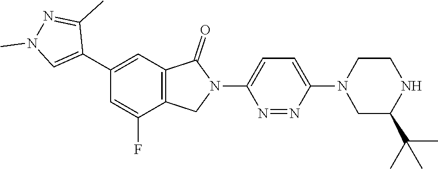 [SMILES: Cc1nn(C)cc1-c1cc(F)c2c(c1)C(=O)N(c1ccc(N3CCN[C@@H](C(C)(C)C)C3)nn1)C2] | (S)-2-(6-(3-(tert-butyl)piperazin-1- yl)pyridazin-3-yl)-6-(1,3-dimethyl- 1H-pyrazol-4-yl)-4- fluoroisoindolin-1-one |
| 296 | 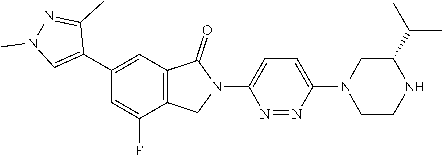 [SMILES: Cc1nn(C)cc1-c1cc(F)c2c(c1)C(=O)N(c1ccc(N3CCN[C@@H](C(C)C)C3)nn1)C2] | (S)-6-(1,3-dimethyl-1H-pyrazol-4- yl)-4-fluoro-2-(6-(3- isopropylpiperazin-1-yl)pyridazin- 3-yl)isoindolin-1-one |
| 297 | 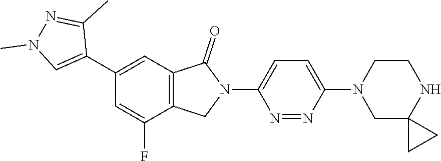 [SMILES: Cc1nn(C)cc1-c1cc(F)c2c(c1)C(=O)N(c1ccc(N3CCNC4(CC4)C3)nn1)C2] | 2-(6-(4,7-diazaspiro[2.5]octan-7- yl)pyridazin-3-yl)-6-(1,3-dimethyl- 1H-pyrazol-4-yl)-4- fluoroisoindolin-1-one |
| 298 | 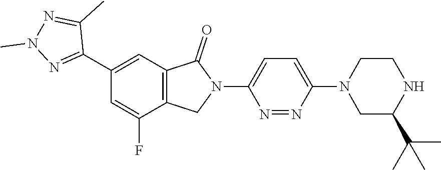 [SMILES: Cc1nn(C)nc1-c1cc(F)c2c(c1)C(=O)N(c1ccc(N3CCN[C@@H](C(C)(C)C)C3)nn1)C2] | (S)-2-(6-(3-(tert-butyl)piperazin-1- yl)pyridazin-3-yl)-6-(2,5-dimethyl- 2H-1,2,3-triazol-4-yl)-4- fluoroisoindolin-1-one |
| 299 | 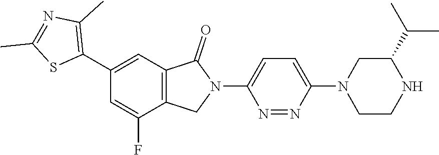 [SMILES: Cc1nc(C)c(-c2cc(F)c3c(c2)C(=O)N(c2ccc(N4CCN[C@@H](C(C)C)C4)nn2)C3)s1] | (S)-6-(2,4-dimethylthiazol-5-yl)-4- fluoro-2-(6-(3-isopropylpiperazin- 1-yl)pyridazin-3-yl)isoindolin-1- one |
| 300 | 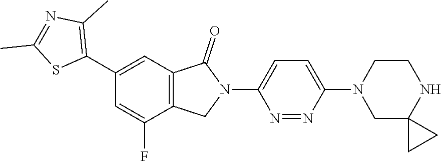 [SMILES: Cc1nc(C)c(-c2cc(F)c3c(c2)C(=O)N(c2ccc(N4CCNC5(CC5)C4)nn2)C3)s1] | 2-(6-(4,7-diazaspiro[2.5]octan-7- yl)pyridazin-3-yl)-6-(2,4- dimethylthiazol-5-yl)-4- fluoroisoindolin-1-one |
| 301 | 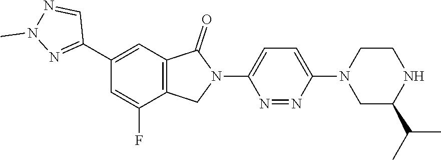 [SMILES: CC(C)[C@H]1CN(c2ccc(N3Cc4c(F)cc(-c5cnn(C)n5)cc4C3=O)nn2)CCN1] | (S)-4-fluoro-2-(6-(3- isopropylpiperazin-1-yl)pyridazin- 3-yl)-6-(2-methyl-2H-1,2,3- triazol-4-yl)isoindolin-1-one |
| 302 | 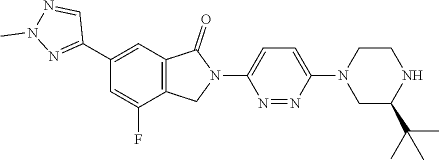 [SMILES: Cn1ncc(-c2cc(F)c3c(c2)C(=O)N(c2ccc(N4CCN[C@@H](C(C)(C)C)C4)nn2)C3)n1] | (S)-2-(6-(3-(tert-butyl)piperazin-1- yl)pyridazin-3-yl)-4-fluoro-6-(2- methyl-2H-1,2,3-triazol-4- yl)isoindolin-1-one |
| 304 | 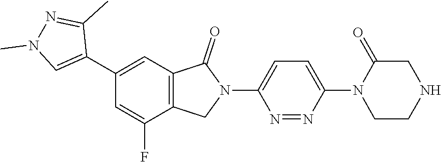 [SMILES: Cc1nn(C)cc1-c1cc(F)c2c(c1)C(=O)N(c1ccc(N3CCNCC3=O)nn1)C2] | 6-(1,3-dimethyl-1H-pyrazol-4-yl)- 4-fluoro-2-(6-(2-oxopiperazin-1- yl)pyridazin-3-yl)isoindolin-1-one |
| 305 | 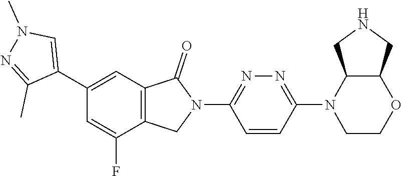 [SMILES: Cc1nn(C)cc1-c1cc(F)c2c(c1)C(=O)N(c1ccc(N3CCO[C@@H]4CNC[C@@H]43)nn1)C2] | 6-(1,3-dimethyl-1H-pyrazol-4-yl)- 4-fluoro-2-(6-((4aS,7aR)- hexahydropyrrolo[3,4- b][1,4]oxazin-4(4aH)-yl)pyridazin- 3-yl)isoindolin-1-one |
| 306 | 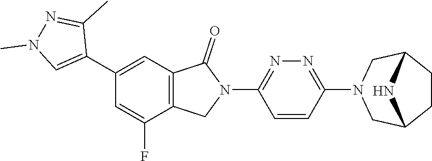 [SMILES: Cc1nn(C)cc1-c1cc(F)c2c(c1)C(=O)N(c1ccc(N3C[C@H]4CC[C@@H](C3)N4)nn1)C2] | 2-(6-((1R,5S)-3,8- diazabicyclo[3.2.1]octan-3- yl)pyridazin-3-yl)-6-(1,3-dimethyl- 1H-pyrazol-4-yl)-4- fluoroisoindolin-1-one |
| 344 | 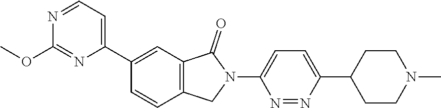 | 6-(2-methoxypyrimidin-4-yl)-2-(6- (1-methylpiperidin-4-yl)pyridazin- 3-yl)isoindolin-1-one |
| 345 | 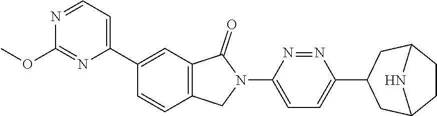 [SMILES: COc1nccc(-c2ccc3c(c2)C(=O)N(c2ccc(C4CC5CCC(C4)N5)nn2)C3)n1] | 2-(6-(8-azabicyclo[3.2.1]octan-3- yl)pyridazin-3-yl)-6-(2- methoxypyrimidin-4-yl)isoindolin- 1-one |
| 361 | 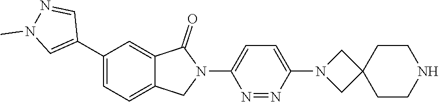 [SMILES: Cn1cc(-c2ccc3c(c2)C(=O)N(c2ccc(N4CC5(CCNCC5)C4)nn2)C3)cn1] | 2-(6-(2,7-diazaspiro[3.5]nonan-2- yl)pyridazin-3-yl)-6-(1-methyl- 1H-pyrazol-4-yl)isoindolin-1-one |
| 374 | 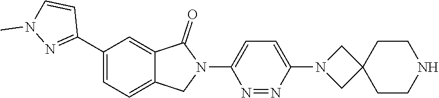 [SMILES: Cn1ccc(-c2ccc3c(c2)C(=O)N(c2ccc(N4CC5(CCNCC5)C4)nn2)C3)n1] | 2-(6-(2,7-diazaspiro[3.5]nonan-2- yl)pyridazin-3-yl)-6-(1-methyl- 1H-pyrazol-3-yl)isoindolin-1-one |
| 379 | 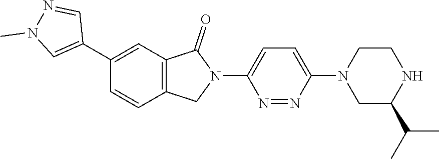 [SMILES: CC(C)[C@H]1CN(c2ccc(N3Cc4ccc(-c5cnn(C)c5)cc4C3=O)nn2)CCN1] | (S)-2-(6-(3-isopropylpiperazin-1- yl)pyridazin-3-yl)-6-(1-methyl- 1H-pyrazol-4-yl)isoindolin-1-one |
| 380 | 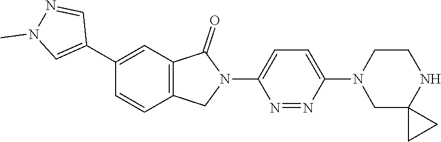 [SMILES: Cn1cc(-c2ccc3c(c2)C(=O)N(c2ccc(N4CCNC5(CC5)C4)nn2)C3)cn1] | 2-(6-(4,7-diazaspiro[2.5]octan-7- yl)pyridazin-3-yl)-6-(1-methyl- 1H-pyrazol-4-yl)isoindolin-1-one |
| 384 | 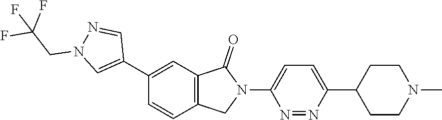 [SMILES: CN1CCC(c2ccc(N3Cc4ccc(-c5cnn(CC(F)(F)F)c5)cc4C3=O)nn2)CC1] | 2-(6-(1-methylpiperidin-4- yl)pyridazin-3-yl)-6-(1-(2,2,2- trifluoroethyl)-1H-pyrazol-4- yl)isoindolin-1-one |
| 388 | 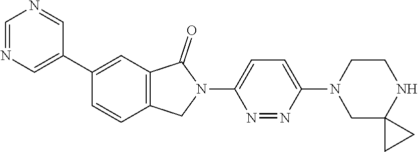 [SMILES: O=C1c2cc(-c3cncnc3)ccc2CN1c1ccc(N2CCNC3(CC3)C2)nn1] | 2-(6-(4,7-diazaspiro[2.5]octan-7- yl)pyridazin-3-yl)-6-(pyrimidin-5- yl)isoindolin-1-one |
| 389 | 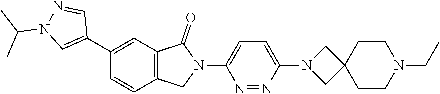 [SMILES: CCN1CCC2(CC1)CN(c1ccc(N3Cc4ccc(-c5cnn(C(C)C)c5)cc4C3=O)nn1)C2] | 2-(6-(7-ethyl-2,7- diazaspiro[3.5]nonan-2- yl)pyridazin-3-yl)-6-(1-isopropyl- 1H-pyrazol-4-yl)isoindolin-1-one |
| 390 | 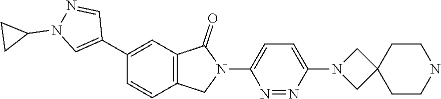 [SMILES: O=C1c2cc(-c3cnn(C4CC4)c3)ccc2CN1c1ccc(N2CC3(CCNCC3)C2)nn1] | 6-(1-cyclopropyl-1H-pyrazol-4- yl)-2-(6-(7-ethyl-2,7- diazaspiro[3.5]nonan-2- yl)pyridazin-3-yl)isoindolin-1-one |
| 391 | 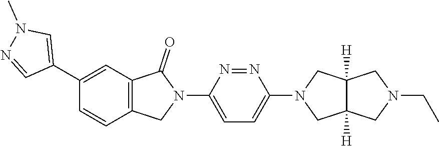 [SMILES: CCN1C[C@@H]2CN(c3ccc(N4Cc5ccc(-c6cnn(C)c6)cc5C4=O)nn3)C[C@@H]2C1] | 2-(6-((3aR,6aS)-5- ethylhexahydropyrrolo[3,4- c]pyrrol-2(1H)-yl)pyridazin-3-yl)- 6-(1-methyl-1H-pyrazol-4- yl)isoindolin-1-one |
| 394 | 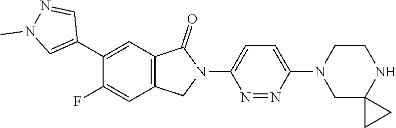 [SMILES: Cn1cc(-c2cc3c(cc2F)CN(c2ccc(N4CCNC5(CC5)C4)nn2)C3=O)cn1] | 2-(6-(4,7-diazaspiro[2.5]octan-7- yl)pyridazin-3-yl)-5-fluoro-6-(1- methyl-1H-pyrazol-4- yl)isoindolin-1-one |
| 395 | 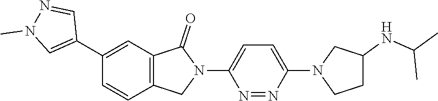 [SMILES: CC(C)NC1CCN(c2ccc(N3Cc4ccc(-c5cnn(C)c5)cc4C3=O)nn2)C1] | 2-(6-(3- (isopropylamino)pyrrolidin-1- yl)pyridazin-3-yl)-6-(1-methyl- 1H-pyrazol-4-yl)isoindolin-1-one |
| 399 | 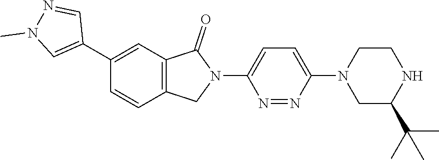 [SMILES: Cn1cc(-c2ccc3c(c2)C(=O)N(c2ccc(N4CCN[C@@H](C(C)(C)C)C4)nn2)C3)cn1] | (S)-2-(6-(3-(tert-butyl)piperazin-1- yl)pyridazin-3-yl)-6-(1-methyl- 1H-pyrazol-4-yl)isoindolin-1-one |
| 400 | 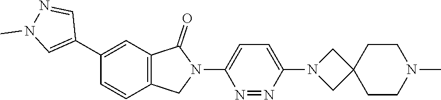 | 2-(6-(7-ethyl-2,7- diazaspiro[3.5]nonan-2- yl)pyridazin-3-yl)-6-(1-methyl- 1H-pyrazol-4-yl)isoindolin-1-one |
| 401 | 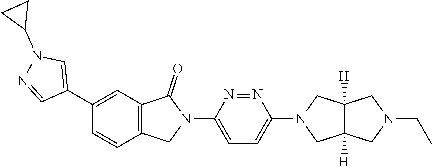 [SMILES: CCN1C[C@@H]2CN(c3ccc(N4Cc5ccc(-c6cnn(C7CC7)c6)cc5C4=O)nn3)C[C@@H]2C1] | 6-(1-cyclopropyl-1H-pyrazol-4- yl)-2-(6-((3aR,6aS)-5- ethylhexahydropyrrolo[3,4- c]pyrrol-2(1H)-yl)pyridazin-3- yl)isoindolin-1-one |
| 402 | 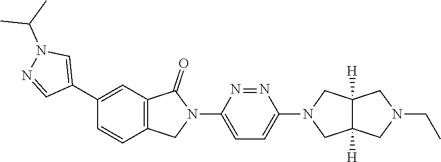 [SMILES: CCN1C[C@@H]2CN(c3ccc(N4Cc5ccc(-c6cnn(C(C)C)c6)cc5C4=O)nn3)C[C@@H]2C1] | 2-(6-((3aR,6aS)-5- ethylhexahydropyrrolo[3,4- c]pyrrol-2(1H)-yl)pyridazin-3-yl)- 6-(1-isopropyl-1H-pyrazol-4- yl)isoindolin-1-one |
| 404 | 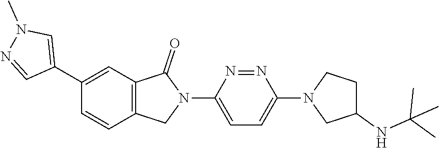 [SMILES: Cn1cc(-c2ccc3c(c2)C(=O)N(c2ccc(N4CCC(NC(C)(C)C)C4)nn2)C3)cn1] | 2-(6-(3-(tert- butylamino)pyrrolidin-1- yl)pyridazin-3-yl)-6-(1-methyl- 1H-pyrazol-4-yl)isoindolin-1-one |
| 405 | 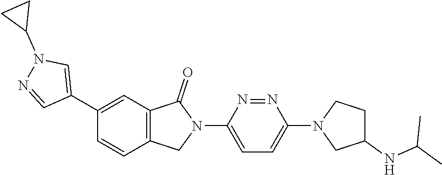 [SMILES: CC(C)NC1CCN(c2ccc(N3Cc4ccc(-c5cnn(C6CC6)c5)cc4C3=O)nn2)C1] | 6-(1-cyclopropyl-1H-pyrazol-4- yl)-2-(6-(3- (isopropylamino)pyrrolidin-1- yl)pyridazin-3-yl)isoindolin-1-one |
| 407 | 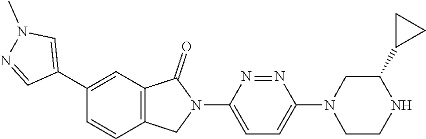 [SMILES: Cn1cc(-c2ccc3c(c2)C(=O)N(c2ccc(N4CCN[C@@H](C5CC5)C4)nn2)C3)cn1] | (S)-2-(6-(3-cyclopropylpiperazin- 1-yl)pyridazin-3-yl)-6-(1-methyl- 1H-pyrazol-4-yl)isoindolin-1-one |
| 408 | 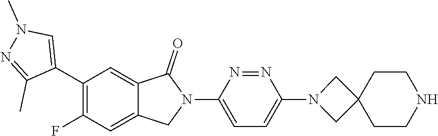 [SMILES: Cc1nn(C)cc1-c1cc2c(cc1F)CN(c1ccc(N3CC4(CCNCC4)C3)nn1)C2=O] | 2-(6-(2,7-diazaspiro[3.5]nonan-2- yl)pyridazin-3-yl)-6-(1,3-dimethyl- 1H-pyrazol-4-yl)-5- fluoroisoindolin-1-one |
| 409 | 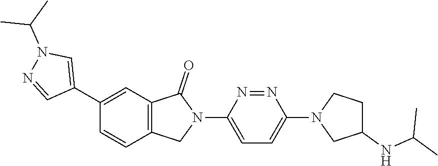 [SMILES: CC(C)NC1CCN(c2ccc(N3Cc4ccc(-c5cnn(C(C)C)c5)cc4C3=O)nn2)C1] | 6-(1-isopropyl-1H-pyrazol-4-yl)-2- (6-(3-(isopropylamino)pyrrolidin- 1-yl)pyridazin-3-yl)isoindolin-1- one |
| 412 | 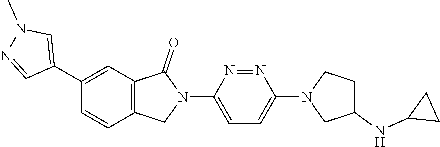 [SMILES: Cn1cc(-c2ccc3c(c2)C(=O)N(c2ccc(N4CCC(NC5CC5)C4)nn2)C3)cn1] | 2-(6-(3- (cyclopropylamino)pyrrolidin-1- yl)pyridazin-3-yl)-6-(1-methyl- 1H-pyrazol-4-yl)isoindolin-1-one |
| 414 | 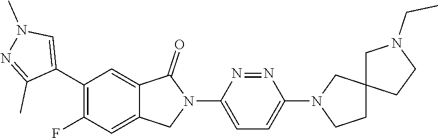 [SMILES: CCN1CCC2(CCN(c3ccc(N4Cc5cc(F)c(-c6cn(C)nc6C)cc5C4=O)nn3)C2)C1] | 6-(1,3-dimethyl-1H-pyrazol-4-yl)- 2-(6-(7-ethyl-2,7- diazaspiro[4.4]nonan-2- yl)pyridazin-3-yl)-5- fluoroisoindolin-1-one |
| 501 | 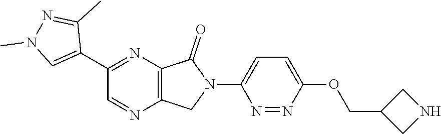 [SMILES: Cc1nn(C)cc1-c1cnc2c(n1)C(=O)N(c1ccc(OCC3CNC3)nn1)C2] | 6-(6-(azetidin-3- ylmethoxy)pyridazin-3-yl)-3-(1,3- dimethyl-1H-pyrazol-4-yl)-6,7- dihydro-5H-pyrrolo[3,4-b]pyrazin- 5-one |
| 510 | 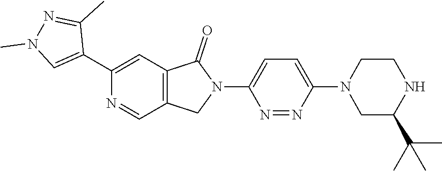 [SMILES: Cc1nn(C)cc1-c1cc2c(cn1)CN(c1ccc(N3CCN[C@@H](C(C)(C)C)C3)nn1)C2=O] | (S)-2-(6-(3-(tert-butyl)piperazin-1- yl)pyridazin-3-yl)-6-(1,3-dimethyl- 1H-pyrazol-4-yl)-2,3-dihydro-1H- pyrrolo[3,4-c]pyridin-1-one |
| 511 | 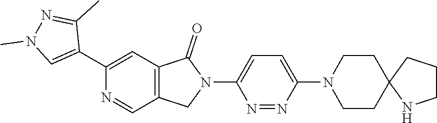 [SMILES: Cc1nn(C)cc1-c1cc2c(cn1)CN(c1ccc(N3CCC4(CCCN4)CC3)nn1)C2=O] | 2-(6-(1,8-diazaspiro[4.5]decan-8- yl)pyridazin-3-yl)-6-(1,3-dimethyl- 1H-pyrazol-4-yl)-2,3-dihydro-1H- pyrrolo[3,4-c]pyridin-1-one |
| 512 | 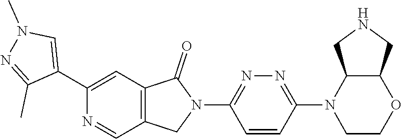 [SMILES: Cc1nn(C)cc1-c1cc2c(cn1)CN(c1ccc(N3CCO[C@@H]4CNC[C@@H]43)nn1)C2=O] | 6-(1,3-dimethyl-1H-pyrazol-4-yl)- 2-(6-((4aS,7aR)- hexahydropyrrolo[3,4- b][1,4]oxazin-4(4aH)-yl)pyridazin- 3-yl)-2,3-dihydro-1H-pyrrolo[3,4- c]pyridin-1-one |
| 513 | 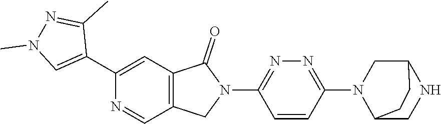 [SMILES: Cc1nn(C)cc1-c1cc2c(cn1)CN(c1ccc(N3CC4CCC3CN4)nn1)C2=O] | 2-(6-(2,5-diazabicyclo[2.2.2]octan- 2-yl)pyridazin-3-yl)-6-(1,3- dimethyl-1H-pyrazol-4-yl)-2,3- dihydro-1H-pyrrolo[3,4-c]pyridin- 1-one |
| 514 | 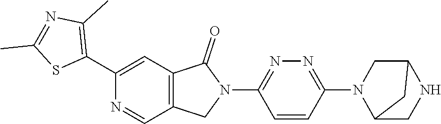 [SMILES: Cc1nc(C)c(-c2cc3c(cn2)CN(c2ccc(N4CC5CC4CN5)nn2)C3=O)s1] | 2-(6-(2,5- diazabicyclo[2.2.1]heptan-2- yl)pyridazin-3-yl)-6-(2,4- dimethylthiazol-5-yl)-2,3-dihydro- 1H-pyrrolo[3,4-c]pyridin-1-one |
| 515 | 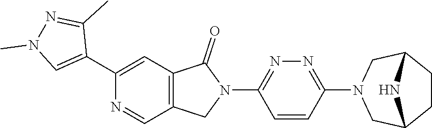 [SMILES: Cc1nn(C)cc1-c1cc2c(cn1)CN(c1ccc(N3C[C@H]4CC[C@@H](C3)N4)nn1)C2=O] | 2-(6-((1R,5S)-3,8- diazabicyclo[3.2.1]octan-3- yl)pyridazin-3-yl)-6-(1,3-dimethyl- 1H-pyrazol-4-yl)-2,3-dihydro-1H- pyrrolo[3,4-c]pyridin-1-one |
| 516 | 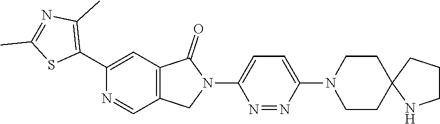 [SMILES: Cc1nc(C)c(-c2cc3c(cn2)CN(c2ccc(N4CCC5(CCCN5)CC4)nn2)C3=O)s1] | 2-(6-(1,8-diazaspiro[4.5]decan-8- yl)pyridazin-3-yl)-6-(2,4- dimethylthiazol-5-yl)-2,3-dihydro- 1H-pyrrolo[3,4-c]pyridin-1-one |
| 517 | 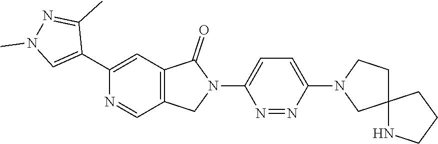 [SMILES: Cc1nn(C)cc1-c1cc2c(cn1)CN(c1ccc(N3CCC4(CCCN4)C3)nn1)C2=O] | 2-(6-(1,7-diazaspiro[4.4]nonan-7- yl)pyridazin-3-yl)-6-(1,3-dimethyl- 1H-pyrazol-4-yl)-2,3-dihydro-1H- pyrrolo[3,4-c]pyridin-1-one |
| 518 | 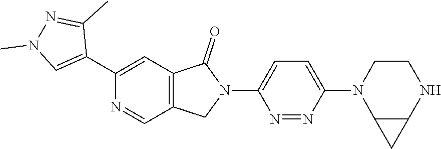 [SMILES: Cc1nn(C)cc1-c1cc2c(cn1)CN(c1ccc(N3CCNC4CC43)nn1)C2=O] | 2-(6-(2,5- diazabicyclo[4.1.0]heptan-2- yl)pyridine151e-3-yl)-6-(1,3- dimethyl-1H-pyrazol-4-yl)-2,3- dihydro-1H-pyrrolo[3,4- c]pyridine-1-one |
| 519 | 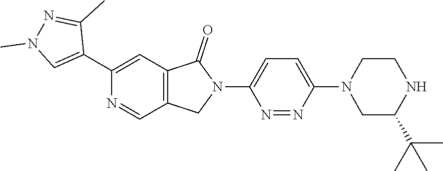 [SMILES: Cc1nn(C)cc1-c1cc2c(cn1)CN(c1ccc(N3CCN[C@H](C(C)(C)C)C3)nn1)C2=O] | (R)-2-(6-(3-(tert-butyl)piperazin-1- yl)pyridazin-3-yl)-6-(1,3-dimethyl- 1H-pyrazol-4-yl)-2,3-dihydro-1H- pyrrolo[3,4-c]pyridin-1-one |
| 520 | 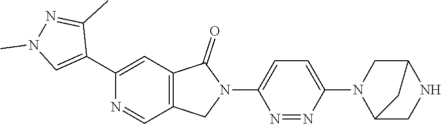 [SMILES: Cc1nn(C)cc1-c1cc2c(cn1)CN(c1ccc(N3CC4CC3CN4)nn1)C2=O] | 2-(6-(2,5- diazabicyclo[2.2.1]heptan-2- yl)pyridazin-3-yl)-6-(1,3-dimethyl- 1H-pyrazol-4-yl)-2,3-dihydro-1H- pyrrolo[3,4-c]pyridin-1-one |
| 521 | 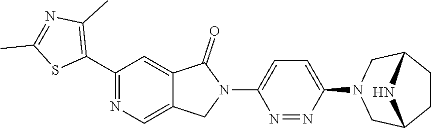 | 2-(6-((1R,5S)-3,8- diazabicyclo[3.2.1]octan-3- yl)pyridazin-3-yl)-6-(2,4- dimethylthiazol-5-yl)-2,3-dihydro- 1H-pyrrolo[3,4-c]pyridin-1-one |
| 522 | 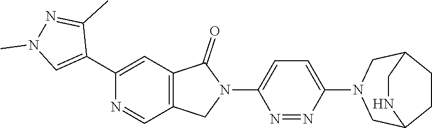 [SMILES: Cc1nn(C)cc1-c1cc2c(cn1)CN(c1ccc(N3CC4CCC(C3)NC4)nn1)C2=O] | 2-(6-(3,6- diazabicyclo[3.2.2]nonan-3- yl)pyridazin-3-yl)-6-(1,3-dimethyl- 1H-pyrazol-4-yl)-2,3-dihydro-1H- pyrrolo[3,4-c]pyridin-1-one |
| 523 | 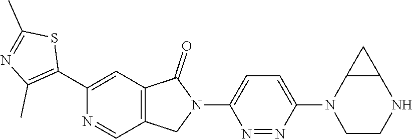 [SMILES: Cc1nc(C)c(-c2cc3c(cn2)CN(c2ccc(N4CCNC5CC54)nn2)C3=O)s1] | 2-(6-(2,5- diazabicyclo[4.1.0]heptan-2- yl)pyridazin-3-yl)-6-(2,4- dimethylthiazol-5-yl)-2,3-dihydro- 1H-pyrrolo[3,4-c]pyridin-1-one |
| 524 | 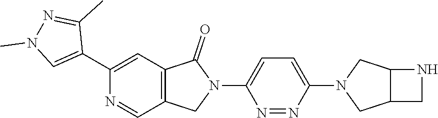 [SMILES: Cc1nn(C)cc1-c1cc2c(cn1)CN(c1ccc(N3CC4CNC4C3)nn1)C2=O] | 2-(6-(3,6- diazabicyclo[3.2.0]heptan-3- yl)pyridazin-3-yl)-6-(1,3-dimethyl- 1H-pyrazol-4-yl)-2,3-dihydro-1H- pyrrolo[3,4-c]pyridin-1-one |
| 525 | 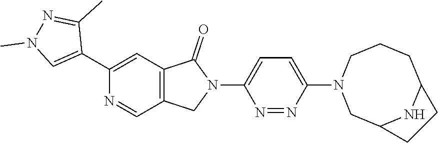 [SMILES: Cc1nn(C)cc1-c1cc2c(cn1)CN(c1ccc(N3CCCC4CCC(C3)N4)nn1)C2=O] | 2-(6-(3,9- diazabicyclo[4.2.1]nonan-3- yl)pyridazin-3-yl)-6-(1,3-dimethyl- 1H-pyrazol-4-yl)-2,3-dihydro-1H- pyrrolo[3,4-c]pyridin-1-one Chemical Formula: C23H26N8O Molecular Weight: 430.5160 |
| 526 | 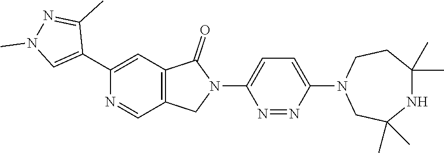 | 6-(1,3-dimethyl-1H-pyrazol-4-yl)- 2-(6-(3,3,5,5-tetramethyl-1,4- diazepan-1-yl)pyridazin-3-yl)-2,3- dihydro-1H-pyrrolo[3,4-c]pyridin- 1-one |
| 527 | 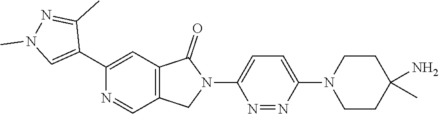 | 2-(6-(4-amino-4-methylpiperidin- 1-yl)pyridazin-3-yl)-6-(1,3- dimethyl-1H-pyrazol-4-yl)-2,3- dihydro-1H-pyrrolo[3,4-c]pyridin- 1-one |
| 528 | 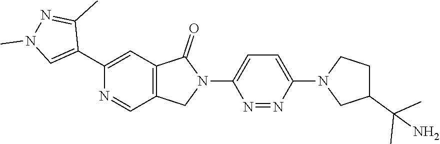 [SMILES: Cc1nn(C)cc1-c1cc2c(cn1)CN(c1ccc(N3CCC(C(C)(C)N)C3)nn1)C2=O] | 2-(6-(3-(2-aminopropan-2- yl)pyrrolidin-1-yl)pyridazin-3-yl)- 6-(1,3-dimethyl-1H-pyrazol-4-yl)- 2,3-dihydro-1H-pyrrolo[3,4- c]pyridin-1-one |
| 529 | 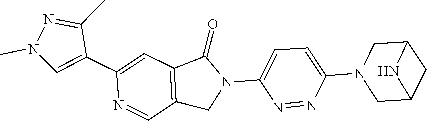 [SMILES: Cc1nn(C)cc1-c1cc2c(cn1)CN(c1ccc(N3CC4CC(C3)N4)nn1)C2=O] | 2-(6-(3,6- diazabicyclo[3.1.1]heptan-3- yl)pyridazin-3-yl)-6-(1,3-dimethyl- 1H-pyrazol-4-yl)-2,3-dihydro-1H- pyrrolo[3,4-c]pyridin-1-one |
| 530 | 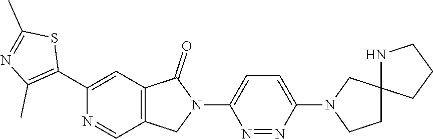 [SMILES: Cc1nc(C)c(-c2cc3c(cn2)CN(c2ccc(N4CCC5(CCCN5)C4)nn2)C3=O)s1] | 2-(6-(1,7-diazaspiro[4.4]nonan-7- yl)pyridazin-3-yl)-6-(2,4- dimethylthiazol-5-yl)-2,3-dihydro- 1H-pyrrolo[3,4-c]pyridin-1-one |
| 531 | 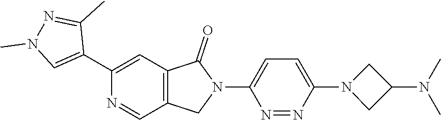 | 6-(1,3-dimethyl-1H-pyrazol-4-yl)- 2-(6-(3-(dimethylamino)azetidin- 1-yl)pyridazin-3-yl)-2,3-dihydro- 1H-pyrrolo[3,4-c]pyridin-1-one |
| 532 | 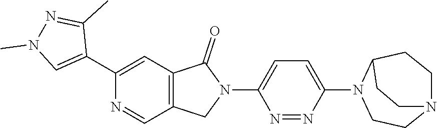 [SMILES: Cc1nn(C)cc1-c1cc2c(cn1)CN(c1ccc(N3CCN4CCC3CC4)nn1)C2=O] | 2-(6-(1,4- diazabicyclo[3.2.2]nonan-4- yl)pyridazin-3-yl)-6-(1,3-dimethyl- 1H-pyrazol-4-yl)-2,3-dihydro-1H- pyrrolo[3,4-c]pyridin-1-one |
| 533 | 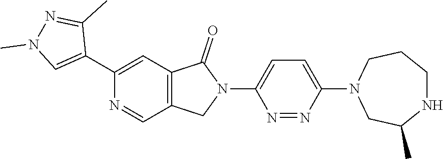 [SMILES: Cc1nn(C)cc1-c1cc2c(cn1)CN(c1ccc(N3CCCN[C@@H](C)C3)nn1)C2=O] | (S)-6-(1,3-dimethyl-1H-pyrazol-4- yl)-2-(6-(3-methyl-1,4-diazepan-1- yl)pyridazin-3-yl)-2,3-dihydro-1H- pyrrolo[3,4-c]pyridin-1-one |
| 534 | 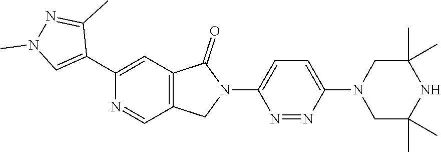 | 6-(1,3-dimethyl-1H-pyrazol-4-yl)- 2-(6-(3,3,5,5-tetramethylpiperazin- 1-yl)pyridazin-3-yl)-2,3-dihydro- 1H-pyrrolo[3,4-c]pyridin-1-one |
| 535 | 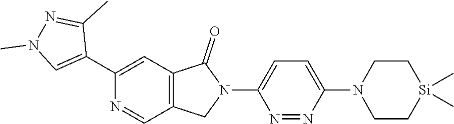 | 2-(6-(4,4-dimethyl-1,4-azasilinan- 1-yl)pyridazin-3-yl)-6-(1,3- dimethyl-1H-pyrazol-4-yl)-2,3- dihydro-1H-pyrrolo[3,4-c]pyridin- 1-one |
| 536 | 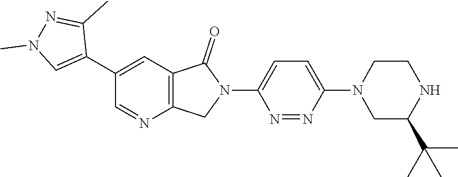 [SMILES: Cc1nn(C)cc1-c1cnc2c(c1)C(=O)N(c1ccc(N3CCN[C@@H](C(C)(C)C)C3)nn1)C2] | (S)-6-(6-(3-(tert-butyl)piperazin-1- yl)pyridazin-3-yl)-3-(1,3-dimethyl- 1H-pyrazol-4-yl)-6,7-dihydro-5H- pyrrolo[3,4-b]pyridin-5-one |
| 537 | 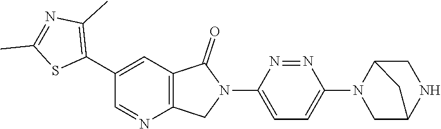 [SMILES: Cc1nc(C)c(-c2cnc3c(c2)C(=O)N(c2ccc(N4CC5CC4CN5)nn2)C3)s1] | 6-(6-(2,5- diazabicyclo[2.2.1]heptan-2- yl)pyridazin-3-yl)-3-(2,4- dimethylthiazol-5-yl)-6,7-dihydro- 5H-pyrrolo[3,4-b]pyridin-5-one |
| 538 | 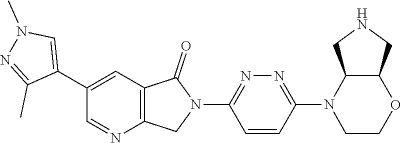 [SMILES: Cc1nn(C)cc1-c1cnc2c(c1)C(=O)N(c1ccc(N3CCO[C@@H]4CNC[C@@H]43)nn1)C2] | 3-(1,3-dimethyl-1H-pyrazol-4-yl)- 6-(6-((4aS,7aR)- hexahydropyrrolo[3,4- b][1,4]oxazin-4(4aH)-yl)pyridazin- 3-yl)-6,7-dihydro-5H-pyrrolo[3,4- b]pyridin-5-one |
| 539 | 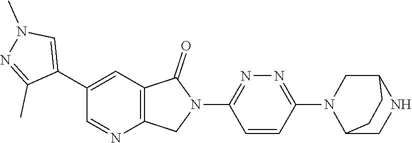 [SMILES: Cc1nn(C)cc1-c1cnc2c(c1)C(=O)N(c1ccc(N3CC4CCC3CN4)nn1)C2] | 6-(6-(2,5-diazabicyclo[2.2.2]octan- 2-yl)pyridazin-3-yl)-3-(1,3- dimethyl-1H-pyrazol-4-yl)-6,7- dihydro-5H-pyrrolo[3,4-b]pyridin- 5-one |
| 540 | 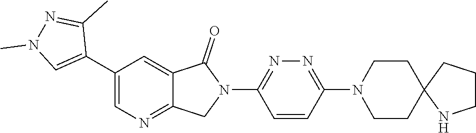 [SMILES: Cc1nn(C)cc1-c1cnc2c(c1)C(=O)N(c1ccc(N3CCC4(CCCN4)CC3)nn1)C2] | 6-(6-(1,8-diazaspiro[4.5]decan-8- yl)pyridazin-3-yl)-3-(1,3-dimethyl- 1H-pyrazol-4-yl)-6,7-dihydro-5H- pyrrolo[3,4-b]pyridin-5-one |
| 541 | 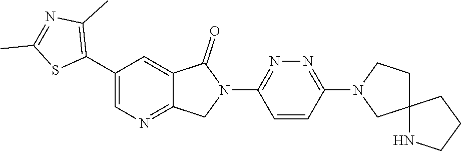 [SMILES: Cc1nc(C)c(-c2cnc3c(c2)C(=O)N(c2ccc(N4CCC5(CCCN5)C4)nn2)C3)s1] | 6-(6-(1,7-diazaspiro[4.4]nonan-7- yl)pyridazin-3-yl)-3-(2,4- dimethylthiazol-5-yl)-6,7-dihydro- 5H-pyrrolo[3,4-b]pyridin-5-one |
| 542 | 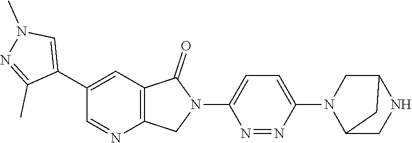 [SMILES: Cc1nn(C)cc1-c1cnc2c(c1)C(=O)N(c1ccc(N3CC4CC3CN4)nn1)C2] | 6-(6-(2,5- diazabicyclo[2.2.1]heptan-2- yl)pyridazin-3-yl)-3-(1,3-dimethyl- 1H-pyrazol-4-yl)-6,7-dihydro-5H- pyrrolo[3,4-b]pyridin-5-one |
| 543 | 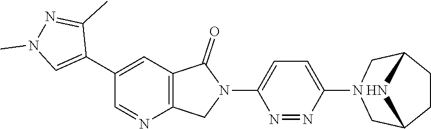 [SMILES: Cc1nn(C)cc1-c1cnc2c(c1)C(=O)N(c1ccc(N3C[C@H]4CC[C@@H](C3)N4)nn1)C2] | 6-(6-((1R,5S)-3,8- diazabicyclo[3.2.1]octan-3- yl)pyridazin-3-yl)-3-(1,3-dimethyl- 1H-pyrazol-4-yl)-6,7-dihydro-5H- pyrrolo[3,4-b]pyridin-5-one |
| 544 | 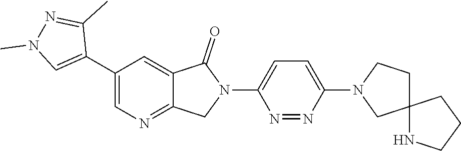 [SMILES: Cc1nn(C)cc1-c1cnc2c(c1)C(=O)N(c1ccc(N3CCC4(CCCN4)C3)nn1)C2] | 6-(6-(1,7-diazaspiro[4.4]nonan-7- yl)pyridazin-3-yl)-3-(1,3-dimethyl- 1H-pyrazol-4-yl)-6,7-dihydro-5H- pyrrolo[3,4-b]pyridin-5-one |
| 545 | 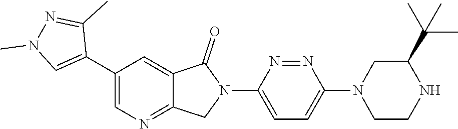 [SMILES: Cc1nn(C)cc1-c1cnc2c(c1)C(=O)N(c1ccc(N3CCN[C@H](C(C)(C)C)C3)nn1)C2] | (R)-6-(6-(3-(tert-butyl)piperazin-1- yl)pyridazin-3-yl)-3-(1,3-dimethyl- 1H-pyrazol-4-yl)-6,7-dihydro-5H- pyrrolo[3,4-b]pyridin-5-one |
| 546 | 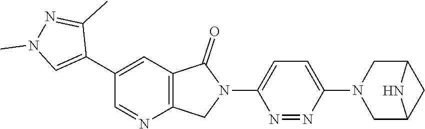 [SMILES: Cc1nn(C)cc1-c1cnc2c(c1)C(=O)N(c1ccc(N3CC4CC(C3)N4)nn1)C2] | 6-(6-(3,6- diazabicyclo[3.1.1]heptan-3- yl)pyridazin-3-yl)-3-(1,3-dimethyl- 1H-pyrazol-4-yl)-6,7-dihydro-5H- pyrrolo[3,4-b]pyridin-5-one |
| 547 | 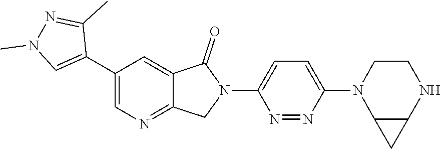 [SMILES: Cc1nn(C)cc1-c1cnc2c(c1)C(=O)N(c1ccc(N3CCNC4CC43)nn1)C2] | 6-(6-(2,5- diazabicyclo[4.1.0]heptan-2- yl)pyridazin-3-yl)-3-(1,3-dimethyl- 1H-pyrazol-4-yl)-6,7-dihydro-5H- pyrrolo[3,4-b]pyridin-5-one |
| 548 | 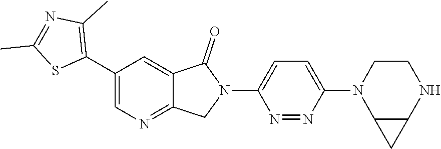 [SMILES: Cc1nc(C)c(-c2cnc3c(c2)C(=O)N(c2ccc(N4CCNC5CC54)nn2)C3)s1] | 6-(6-(2,5- diazabicyclo[4.1.0]heptan-2- yl)pyridazin-3-yl)-3-(2,4- dimethylthiazol-5-yl)-6,7-dihydro- 5H-pyrrolo[3,4-b]pyridin-5-one |
| 549 | 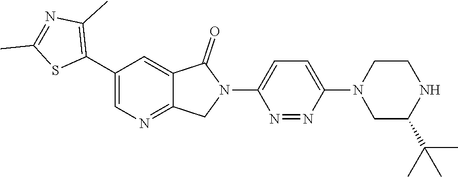 [SMILES: Cc1nc(C)c(-c2cnc3c(c2)C(=O)N(c2ccc(N4CCN[C@H](C(C)(C)C)C4)nn2)C3)s1] | (R)-6-(6-(3-(tert-butyl)piperazin-1- yl)pyridazin-3-yl)-3-(2,4- dimethylthiazol-5-yl)-6,7-dihydro- 5H-pyrrolo[3,4-b]pyridin-5-one |
| 550 | 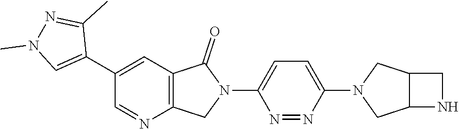 [SMILES: Cc1nn(C)cc1-c1cnc2c(c1)C(=O)N(c1ccc(N3CC4CNC4C3)nn1)C2] | 6-(6-(3,6- diazabicyclo[3.2.0]heptan-3- yl)pyridazin-3-yl)-3-(1,3-dimethyl- 1H-pyrazol-4-yl)-6,7-dihydro-5H- pyrrolo[3,4-b]pyridin-5-one |
| 551 | 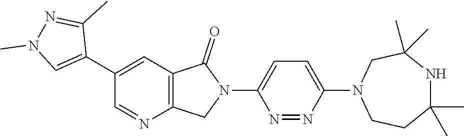 | 3-(1,3-dimethyl-1H-pyrazol-4-yl)- 6-(6-(3,3,5,5-tetramethyl-1,4- diazepan-1-yl)pyridazin-3-yl)-6,7- dihydro-5H-pyrrolo[3,4-b]pyridin- 5-one |
| 552 | 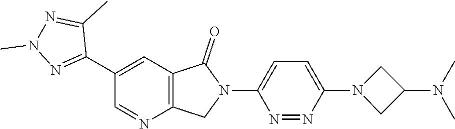 | 3-(2,5-dimethyl-2H-1,2,3-triazol- 4-yl)-6-(6-(3- (dimethylamino)azetidin-1- yl)pyridazin-3-yl)-6,7-dihydro-5H- pyrrolo[3,4-b]pyridin-5-one |
| 553 | 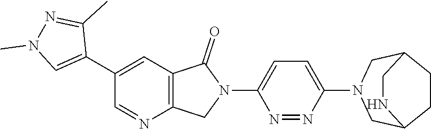 [SMILES: Cc1nn(C)cc1-c1cnc2c(c1)C(=O)N(c1ccc(N3CC4CCC(C3)NC4)nn1)C2] | 6-(6-(3,6- diazabicyclo[3.2.2]nonan-3- yl)pyridazin-3-yl)-3-(1,3-dimethyl- 1H-pyrazol-4-yl)-6,7-dihydro-5H- pyrrolo[3,4-b]pyridin-5-one |
| 554 | 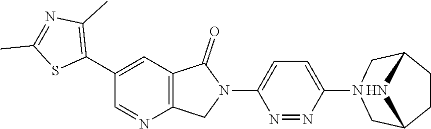 [SMILES: Cc1nc(C)c(-c2cnc3c(c2)C(=O)N(c2ccc(N4C[C@H]5CC[C@@H](C4)N5)nn2)C3)s1] | 6-(6-((1R,5S)-3,8- diazabicyclo[3.2.1]octan-3- yl)pyridazin-3-yl)-3-(2,4- dimethylthiazol-5-yl)-6,7-dihydro- 5H-pyrrolo[3,4-b]pyridin-5-one |
| 555 | 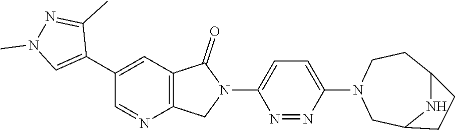 [SMILES: Cc1nn(C)cc1-c1cnc2c(c1)C(=O)N(c1ccc(N3CCC4CCC(C3)N4)nn1)C2] | 6-(6-(3,9- diazabicyclo[4.2.1]nonan-3- yl)pyridazin-3-yl)-3-(1,3-dimethyl- 1H-pyrazol-4-yl)-6,7-dihydro-5H- pyrrolo[3,4-b]pyridin-5-one |
| 556 | 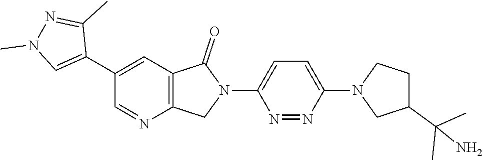 [SMILES: Cc1nn(C)cc1-c1cnc2c(c1)C(=O)N(c1ccc(N3CCC(C(C)(C)N)C3)nn1)C2] | 6-(6-(3-(2-aminopropan-2- yl)pyrrolidin-1-yl)pyridazin-3-yl)- 3-(1,3-dimethyl-1H-pyrazol-4-yl)- 6,7-dihydro-5H-pyrrolo[3,4- b]pyridin-5-one |
| 557 | 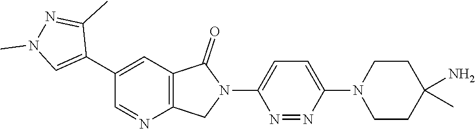 | 6-(6-(4-amino-4-methylpiperidin- 1-yl)pyridazin-3-yl)-3-(1,3- dimethyl-1H-pyrazol-4-yl)-6,7- dihydro-5H-pyrrolo[3,4-b]pyridin- 5-one |
| 558 | 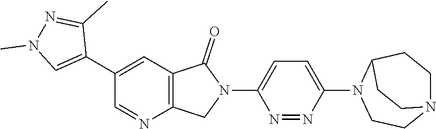 [SMILES: Cc1nn(C)cc1-c1cnc2c(c1)C(=O)N(c1ccc(N3CCN4CCC3CC4)nn1)C2] | 6-(6-(1,4- diazabicyclo[3.2.2]nonan-4- yl)pyridazin-3-yl)-3-(1,3-dimethyl- 1H-pyrazol-4-yl)-6,7-dihydro-5H- pyrrolo[3,4-b]pyridin-5-one |
| 559 | 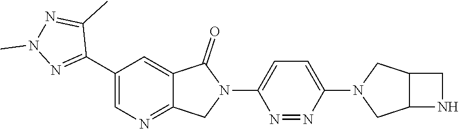 [SMILES: Cc1nn(C)nc1-c1cnc2c(c1)C(=O)N(c1ccc(N3CC4CNC4C3)nn1)C2] | 6-(6-(3,6- diazabicyclo[3.2.0]heptan-3- yl)pyridazin-3-yl)-3-(2,5-dimethyl- 2H-1,2,3-triazol-4-yl)-6,7-dihydro- 5H-pyrrolo[3,4-b]pyridin-5-one |
| 560 | 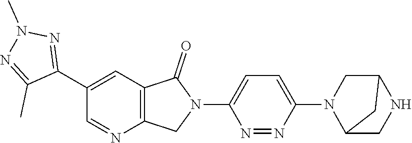 [SMILES: Cc1nn(C)nc1-c1cnc2c(c1)C(=O)N(c1ccc(N3CC4CC3CN4)nn1)C2] | 6-(6-(2,5- diazabicyclo[2.2.1]heptan-2- yl)pyridazin-3-yl)-3-(2,5-dimethyl- 2H-1,2,3-triazol-4-yl)-6,7-dihydro- 5H-pyrrolo[3,4-b]pyridin-5-one |
| 561 | 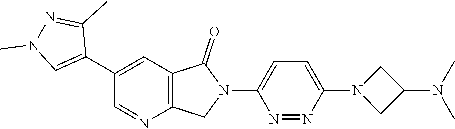 | 3-(1,3-dimethyl-1H-pyrazol-4-yl)- 6-(6-(3-(dimethylamino)azetidin- 1-yl)pyridazin-3-yl)-6,7-dihydro- 5H-pyrrolo[3,4-b]pyridin-5-one |
| 562 | 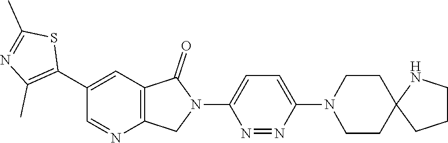 [SMILES: Cc1nc(C)c(-c2cnc3c(c2)C(=O)N(c2ccc(N4CCC5(CCCN5)CC4)nn2)C3)s1] | 6-(6-(1,8-diazaspiro[4.5]decan-8- yl)pyridazin-3-yl)-3-(2,4- dimethylthiazol-5-yl)-6,7-dihydro- 5H-pyrrolo[3,4-b]pyridin-5-one |
| 563 | 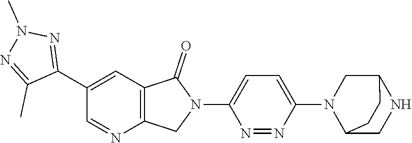 [SMILES: Cc1nn(C)nc1-c1cnc2c(c1)C(=O)N(c1ccc(N3CC4CCC3CN4)nn1)C2] | 6-(6-(2,5-diazabicyclo[2.2.2]octan- 2-yl)pyridazin-3-yl)-3-(2,5- dimethyl-2H-1,2,3-triazol-4-yl)- 6,7-dihydro-5H-pyrrolo[3,4- b]pyridin-5-one |
| 564 | 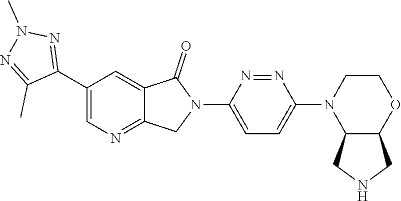 [SMILES: Cc1nn(C)nc1-c1cnc2c(c1)C(=O)N(c1ccc(N3CCO[C@H]4CNC[C@H]43)nn1)C2] | 3-(2,5-dimethyl-2H-1,2,3-triazol- 4-yl)-6-(6-((4aR,7aS)- hexahydropyrrolo[3,4- b][1,4]oxazin-4(4aH)-yl)pyridazin- 3-yl)-6,7-dihydro-5H-pyrrolo[3,4- b]pyridin-5-one |
| 565 | 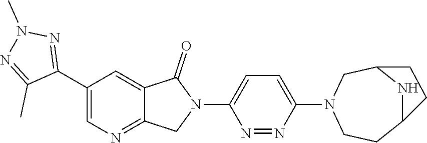 [SMILES: Cc1nn(C)nc1-c1cnc2c(c1)C(=O)N(c1ccc(N3CCC4CCC(C3)N4)nn1)C2] | 6-(6-(3,9- diazabicyclo[4.2.1]nonan-3- yl)pyridazin-3-yl)-3-(2,5-dimethyl- 2H-1,2,3-triazol-4-yl)-6,7-dihydro- 5H-pyrrolo[3,4-b]pyridin-5-one Chemical Formula: C22H25N9O Molecular Weight: 431.5040 |
| 566 | 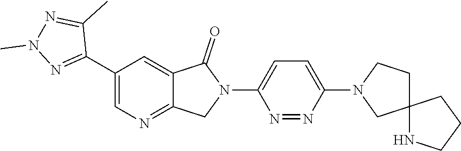 [SMILES: Cc1nn(C)nc1-c1cnc2c(c1)C(=O)N(c1ccc(N3CCC4(CCCN4)C3)nn1)C2] | 6-(6-(1,7-diazaspiro[4.4]nonan-7- yl)pyridazin-3-yl)-3-(2,5-dimethyl- 2H-1,2,3-triazol-4-yl)-6,7-dihydro- 5H-pyrrolo[3,4-b]pyridin-5-one |
| 567 | 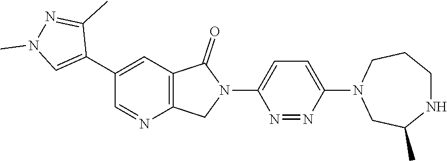 [SMILES: Cc1nn(C)cc1-c1cnc2c(c1)C(=O)N(c1ccc(N3CCCN[C@@H](C)C3)nn1)C2] | (S)-3-(1,3-dimethyl-1H-pyrazol-4- yl)-6-(6-(3-methyl-1,4-diazepan-1- yl)pyridazin-3-yl)-6,7-dihydro-5H- pyrrolo[3,4-b]pyridin-5-one |
| 568 | 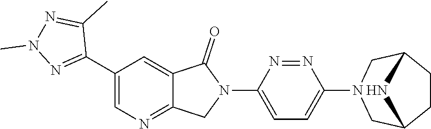 [SMILES: Cc1nn(C)nc1-c1cnc2c(c1)C(=O)N(c1ccc(N3C[C@H]4CC[C@@H](C3)N4)nn1)C2] | 6-(6-((1R,5S)-3,8- diazabicyclo[3.2.1]octan-3- yl)pyridazin-3-yl)-3-(2,5-dimethyl- 2H-1,2,3-triazol-4-yl)-6,7-dihydro- 5H-pyrrolo[3,4-b]pyridin-5-one |
| 569 | 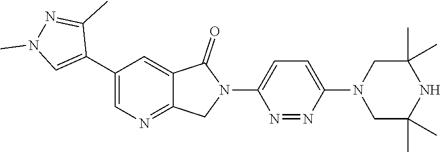 | 3-(1,3-dimethyl-1H-pyrazol-4-yl)- 6-(6-(3,3,5,5-tetramethylpiperazin- 1-yl)pyridazin-3-yl)-6,7-dihydro- 5H-pyrrolo[3,4-b]pyridin-5-one |
| 570 | 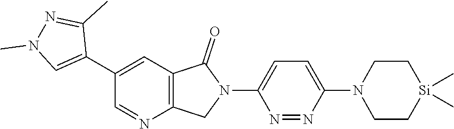 | 6-(6-(4,4-dimethyl-1,4-azasilinan- 1-yl)pyridazin-3-yl)-3-(1,3- dimethyl-1H-pyrazol-4-yl)-6,7- dihydro-5H-pyrrolo[3,4-b]pyridin- 5-one |
| 571 | 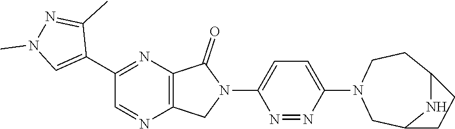 [SMILES: Cc1nn(C)cc1-c1cnc2c(n1)C(=O)N(c1ccc(N3CCC4CCC(C3)N4)nn1)C2] | 6-(6-(3,9- diazabicyclo[4.2.1]nonan-3- yl)pyridazin-3-yl)-3-(1,3-dimethyl- 1H-pyrazol-4-yl)-6,7-dihydro-5H- pyrrolo[3,4-b]pyrazin-5-one |
| 572 | 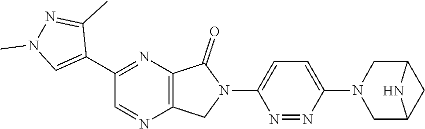 [SMILES: Cc1nn(C)cc1-c1cnc2c(n1)C(=O)N(c1ccc(N3CC4CC(C3)N4)nn1)C2] | 6-(6-(3,6- diazabicyclo[3.1.1]heptan-3- yl)pyridazin-3-yl)-3-(1,3-dimethyl- 1H-pyrazol-4-yl)-6,7-dihydro-5H- pyrrolo[3,4-b]pyrazin-5-one |
| 573 | 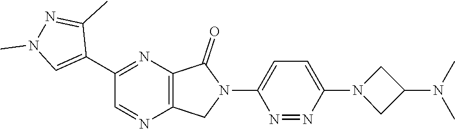 | 3-(1,3-dimethyl-1H-pyrazol-4-yl)- 6-(6-(3-(dimethylamino)azetidin- 1-yl)pyridazin-3-yl)-6,7-dihydro- 5H-pyrrolo[3,4-b]pyrazin-5-one |
| 574 | 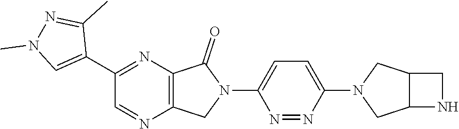 [SMILES: Cc1nn(C)cc1-c1cnc2c(n1)C(=O)N(c1ccc(N3CC4CNC4C3)nn1)C2] | 6-(6-(3,6- diazabicyclo[3.2.0]heptan-3- yl)pyridazin-3-yl)-3-(1,3-dimethyl- 1H-pyrazol-4-yl)-6,7-dihydro-5H- pyrrolo[3,4-b]pyrazin-5-one |
| 575 | 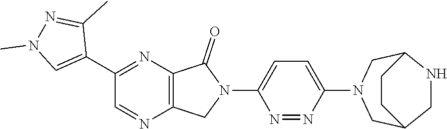 [SMILES: Cc1nn(C)cc1-c1cnc2c(n1)C(=O)N(c1ccc(N3CC4CCC(C3)NC4)nn1)C2] | 6-(6-(3,6- diazabicyclo[3.2.2]nonan-3- yl)pyridazin-3-yl)-3-(1,3-dimethyl- 1H-pyrazol-4-yl)-6,7-dihydro-5H- pyrrolo[3,4-b]pyrazin-5-one |
| 576 | 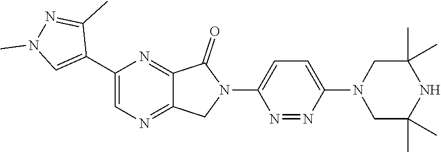 | 3-(1,3-dimethyl-1H-pyrazol-4-yl)- 6-(6-(3,3,5,5-tetramethylpiperazin- 1-yl)pyridazin-3-yl)-6,7-dihydro- 5H-pyrrolo[3,4-b]pyrazin-5-one |
| 577 | 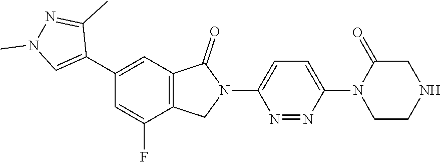 [SMILES: Cc1nn(C)cc1-c1cc(F)c2c(c1)C(=O)N(c1ccc(N3CCNCC3=O)nn1)C2] | 6-(1,3-dimethyl-1H-pyrazol-4-yl)- 4-fluoro-2-(6-(2-oxopiperazin-1- yl)pyridazin-3-yl)isoindolin-1-one |
| 578 | 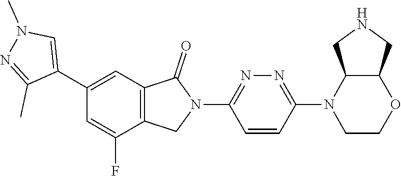 [SMILES: Cc1nn(C)cc1-c1cc(F)c2c(c1)C(=O)N(c1ccc(N3CCO[C@@H]4CNC[C@@H]43)nn1)C2] | 6-(1,3-dimethyl-1H-pyrazol-4-yl)- 4-fluoro-2-(6-((4aS,7aR)- hexahydropyrrolo[3,4- b][1,4]oxazin-4(4aH)-yl)pyridazin- 3-yl)isoindolin-1-one |
| 579 | 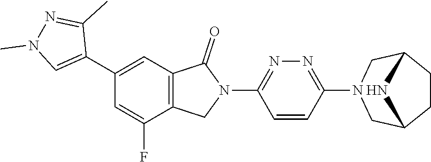 [SMILES: Cc1nn(C)cc1-c1cc(F)c2c(c1)C(=O)N(c1ccc(N3C[C@H]4CC[C@@H](C3)N4)nn1)C2] | 2-(6-((1R,5S)-3,8- diazabicyclo[3.2.1]octan-3- yl)pyridazin-3-yl)-6-(1,3-dimethyl- 1H-pyrazol-4-yl)-4- fluoroisoindolin-1-one |
| 580 | 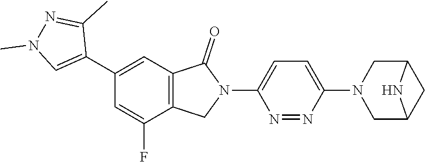 [SMILES: Cc1nn(C)cc1-c1cc(F)c2c(c1)C(=O)N(c1ccc(N3CC4CC(C3)N4)nn1)C2] | 2-(6-(3,6- diazabicyclo[3.1.1]heptan-3- yl)pyridazin-3-yl)-6-(1,3-dimethyl- 1H-pyrazol-4-yl)-4- fluoroisoindolin-1-one |
| 581 | 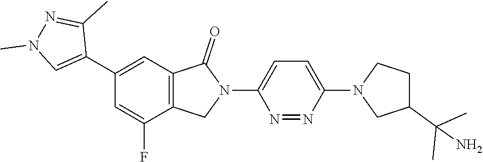 [SMILES: Cc1nn(C)cc1-c1cc(F)c2c(c1)C(=O)N(c1ccc(N3CCC(C(C)(C)N)C3)nn1)C2] | 2-(6-(3-(2-aminopropan-2- yl)pyrrolidin-1-yl)pyridazin-3-yl)- 6-(1,3-dimethyl-1H-pyrazol-4-yl)- 4-fluoroisoindolin-1-one |
| 582 | 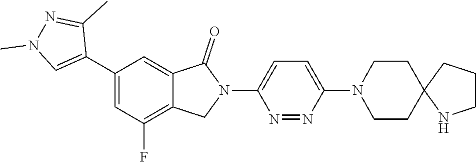 [SMILES: Cc1nn(C)cc1-c1cc(F)c2c(c1)C(=O)N(c1ccc(N3CCC4(CCCN4)CC3)nn1)C2] | 2-(6-(1,8-diazaspiro[4.5]decan-8- yl)pyridazin-3-yl)-6-(1,3-dimethyl- 1H-pyrazol-4-yl)-4- fluoroisoindolin-1-one |
| 583 | 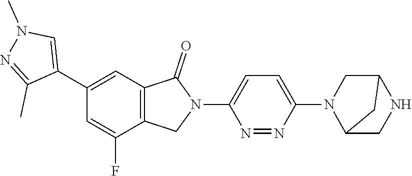 [SMILES: Cc1nn(C)cc1-c1cc(F)c2c(c1)C(=O)N(c1ccc(N3CC4CC3CN4)nn1)C2] | 2-(6-(2,5- diazabicyclo[2.2.1]heptan-2- yl)pyridazin-3-yl)-6-(1,3-dimethyl- 1H-pyrazol-4-yl)-4- fluoroisoindolin-1-one |
| 584 | 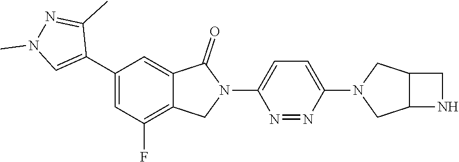 [SMILES: Cc1nn(C)cc1-c1cc(F)c2c(c1)C(=O)N(c1ccc(N3CC4CNC4C3)nn1)C2] | 2-(6-(3,6- diazabicyclo[3.2.0]heptan-3- yl)pyridazin-3-yl)-6-(1,3-dimethyl- 1H-pyrazol-4-yl)-4- fluoroisoindolin-1-one |
| 585 | 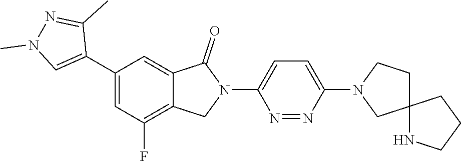 [SMILES: Cc1nn(C)cc1-c1cc(F)c2c(c1)C(=O)N(c1ccc(N3CCC4(CCCN4)C3)nn1)C2] | 2-(6-(1,7-diazaspiro[4.4]nonan-7- yl)pyridazin-3-yl)-6-(1,3-dimethyl- 1H-pyrazol-4-yl)-4- fluoroisoindolin-1-one |
| 586 | 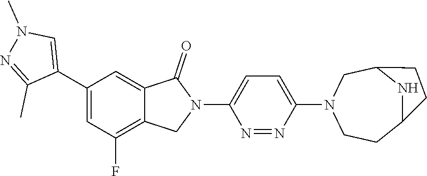 [SMILES: Cc1nn(C)cc1-c1cc(F)c2c(c1)C(=O)N(c1ccc(N3CCC4CCC(C3)N4)nn1)C2] | 2-(6-(3,9- diazabicyclo[4.2.1]nonan-3- yl)pyridazin-3-yl)-6-(1,3-dimethyl- 1H-pyrazol-4-yl)-4- fluoroisoindolin-1-one |
| 587 | 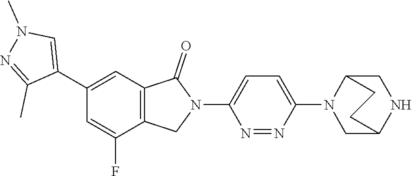 [SMILES: Cc1nn(C)cc1-c1cc(F)c2c(c1)C(=O)N(c1ccc(N3CC4CCC3CN4)nn1)C2] | 2-(6-(2,5-diazabicyclo[2.2.2]octan- 2-yl)pyridazin-3-yl)-6-(1,3- dimethyl-1H-pyrazol-4-yl)-4- fluoroisoindolin-1-one |
| 588 | 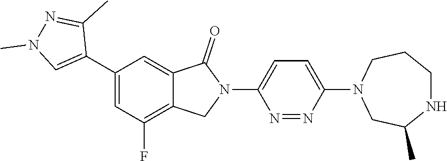 [SMILES: Cc1nn(C)cc1-c1cc(F)c2c(c1)C(=O)N(c1ccc(N3CCCN[C@@H](C)C3)nn1)C2] | (S)-6-(1,3-dimethyl-1H-pyrazol-4- yl)-4-fluoro-2-(6-(3-methyl-1,4- diazepan-1-yl)pyridazin-3- yl)isoindolin-1-one |
| 600 | 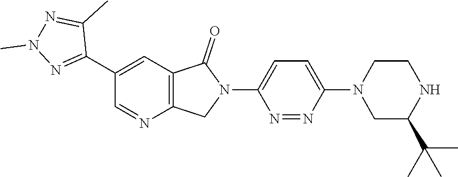 [SMILES: Cc1nn(C)nc1-c1cnc2c(c1)C(=O)N(c1ccc(N3CCN[C@@H](C(C)(C)C)C3)nn1)C2] | (S)-6-(6-(3-(tert-butyl)piperazin-1- yl)pyridazin-3-yl)-3-(2,5-dimethyl- 2H-1,2,3-triazol-4-yl)-6,7-dihydro- 5H-pyrrolo[3,4-b]pyridin-5-one |
| 602 | 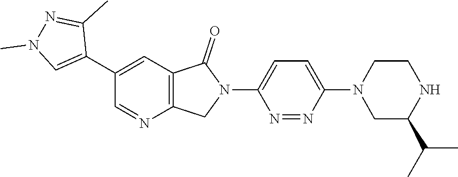 [SMILES: Cc1nn(C)cc1-c1cnc2c(c1)C(=O)N(c1ccc(N3CCN[C@@H](C(C)C)C3)nn1)C2] | (S)-3-(1,3-dimethyl-1H-pyrazol-4- yl)-6-(6-(3-isopropylpiperazin-1- yl)pyridazin-3-yl)-6,7-dihydro-5H- pyrrolo[3,4-b]pyridin-5-one |
| 603 | 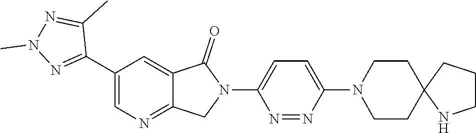 [SMILES: Cc1nn(C)nc1-c1cnc2c(c1)C(=O)N(c1ccc(N3CCC4(CCCN4)CC3)nn1)C2] | 6-(6-(1,8-diazaspiro[4.5]decan-8- yl)pyridazin-3-yl)-3-(2,5-dimethyl- 2H-1,2,3-triazol-4-yl)-6,7-dihydro- 5H-pyrrolo[3,4-b]pyridin-5-one |
| 613 | 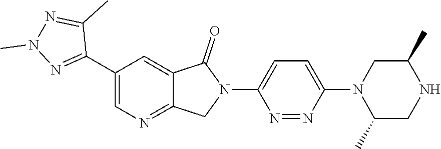 | 3-(2,5-dimethyl-2H-1,2,3-triazol- 4-yl)-6-(6-((2S,5R)-2,5- dimethylpiperazin-1-yl)pyridazin- 3-yl)-6,7-dihydro-5H-pyrrolo[3,4- b]pyridin-5-one |
| 614 | 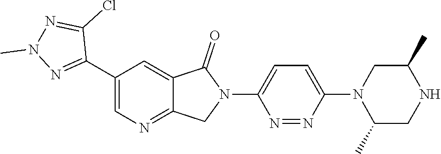 | 3-(5-chloro-2-methyl-2H-1,2,3- triazol-4-yl)-6-(6-((2S,5R)-2,5- dimethylpiperazin-1-yl)pyridazin- 3-yl)-6,7-dihydro-5H-pyrrolo[3,4- b]pyridin-5-one |
| 615 | 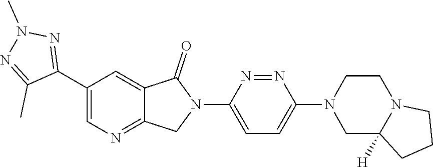 [SMILES: Cc1nn(C)nc1-c1cnc2c(c1)C(=O)N(c1ccc(N3CCN4CCC[C@H]4C3)nn1)C2] | (S)-3-(2,5-dimethyl-2H-1,2,3- triazol-4-yl)-6-(6- (hexahydropyrrolo[1,2-a]pyrazin- 2(1H)-yl)pyridazin-3-yl)-6,7- dihydro-5H-pyrrolo[3,4-b]pyridin- 5-one |
| 616 | 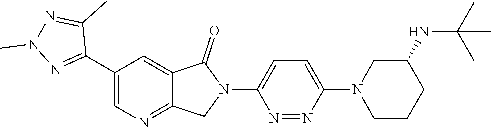 [SMILES: Cc1nn(C)nc1-c1cnc2c(c1)C(=O)N(c1ccc(N3CCC[C@@H](NC(C)(C)C)C3)nn1)C2] | (R)-6-(6-(3-(tert- butylamino)piperidin-1- yl)pyridazin-3-yl)-3-(2,5-dimethyl- 2H-1,2,3-triazol-4-yl)-6,7-dihydro- 5H-pyrrolo[3,4-b]pyridin-5-one |
| 617 | 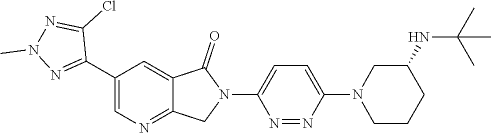 [SMILES: Cn1nc(Cl)c(-c2cnc3c(c2)C(=O)N(c2ccc(N4CCC[C@@H](NC(C)(C)C)C4)nn2)C3)n1] | (R)-6-(6-(3-(tert- butylamino)piperidin-1- yl)pyridazin-3-yl)-3-(5-chloro-2- methyl-2H-1,2,3-triazol-4-yl)-6,7- dihydro-5H-pyrrolo[3,4-b]pyridin- 5-one |
| 618 | 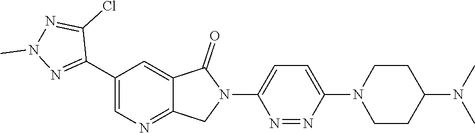 | 3-(5-chloro-2-methyl-2H-1,2,3- triazol-4-yl)-6-(6-(4- (dimethylamino)piperidin-1- yl)pyridazin-3-yl)-6,7-dihydro-5H- pyrrolo[3,4-b]pyridin-5-one |
| 620 | 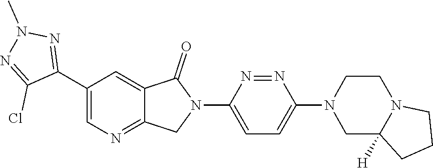 [SMILES: Cn1nc(Cl)c(-c2cnc3c(c2)C(=O)N(c2ccc(N4CCN5CCC[C@H]5C4)nn2)C3)n1] | (S)-3-(5-chloro-2-methyl-2H- 1,2,3-triazol-4-yl)-6-(6- (hexahydropyrrolo[1,2-a]pyrazin- 2(1H)-yl)pyridazin-3-yl)-6,7- dihydro-5H-pyrrolo[3,4-b]pyridin- 5-one |
| 629 | 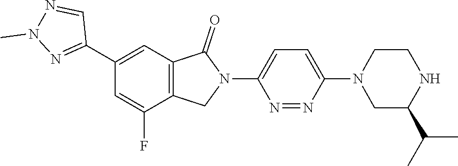 [SMILES: CC(C)[C@H]1CN(c2ccc(N3Cc4c(F)cc(-c5cnn(C)n5)cc4C3=O)nn2)CCN1] | (S)-4-fluoro-2-(6-(3- isopropylpiperazin-1-yl)pyridazin- 3-yl)-6-(2-methyl-2H-1,2,3- triazol-4-yl)isoindolin-1-one |
| 630 | 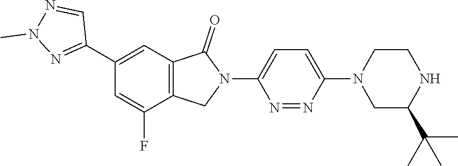 [SMILES: Cn1ncc(-c2cc(F)c3c(c2)C(=O)N(c2ccc(N4CCN[C@@H](C(C)(C)C)C4)nn2)C3)n1] | (S)-2-(6-(3-(tert-butyl)piperazin-1- yl)pyridazin-3-yl)-4-fluoro-6-(2- methyl-2H-1,2,3-triazol-4- yl)isoindolin-1-one |
| 636 | 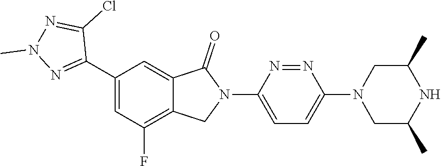 | 6-(5-chloro-2-methyl-2H-1,2,3- triazol-4-yl)-2-(6-((3S,5R)-3,5- dimethylpiperazin-1-yl)pyridazin- 3-yl)-4-fluoroisoindolin-1-one |
| 637 | 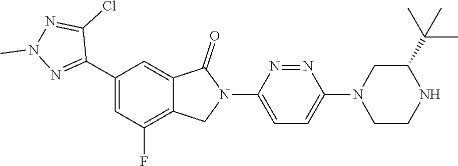 [SMILES: Cn1nc(Cl)c(-c2cc(F)c3c(c2)C(=O)N(c2ccc(N4CCN[C@@H](C(C)(C)C)C4)nn2)C3)n1] | (S)-2-(6-(3-(tert-butyl)piperazin-1- yl)pyridazin-3-yl)-6-(5-chloro-2- methyl-2H-1,2,3-triazol-4-yl)-4- fluoroisoindolin-1-one |
| 638 | 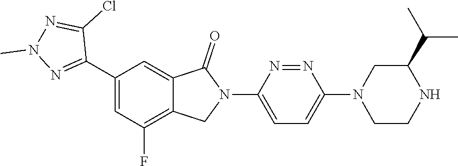 [SMILES: CC(C)[C@@H]1CN(c2ccc(N3Cc4c(F)cc(-c5nn(C)nc5Cl)cc4C3=O)nn2)CCN1] | (R)-6-(5-chloro-2-methyl-2H- 1,2,3-triazol-4-yl)-4-fluoro-2-(6- (3-isopropylpiperazin-1- yl)pyridazin-3-yl)isoindolin-1-one |
| 639 | 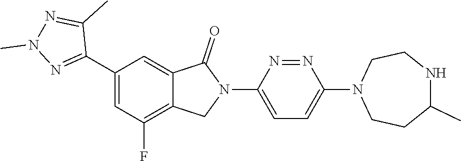 | 6-(2,5-dimethyl-2H-1,2,3-triazol- 4-yl)-4-fluoro-2-(6-(5-methyl-1,4- diazepan-1-yl)pyridazin-3- yl)isoindolin-1-one |
| 640 | 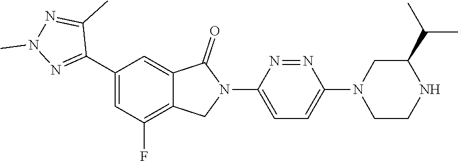 [SMILES: Cc1nn(C)nc1-c1cc(F)c2c(c1)C(=O)N(c1ccc(N3CCN[C@H](C(C)C)C3)nn1)C2] | (R)-6-(2,5-dimethyl-2H-1,2,3- triazol-4-yl)-4-fluoro-2-(6-(3- isopropylpiperazin-1-yl)pyridazin- 3-yl)isoindolin-1-one |
| 641 | 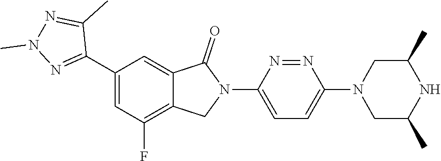 | 6-(2,5-dimethyl-2H-1,2,3-triazol- 4-yl)-2-(6-((3S,5R)-3,5- dimethylpiperazin-1-yl)pyridazin- 3-yl)-4-fluoroisoindolin-1-one |
| 642 | 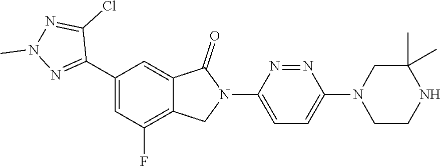 | 6-(5-chloro-2-methyl-2H-1,2,3- triazol-4-yl)-2-(6-(3,3- dimethylpiperazin-1-yl)pyridazin- 3-yl)-4-fluoroisoindolin-1-one |
| 643 | 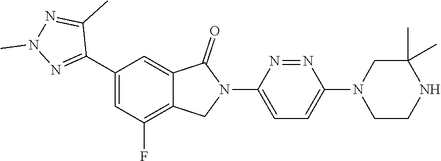 | 6-(2,5-dimethyl-2H-1,2,3-triazol- 4-yl)-2-(6-(3,3-dimethylpiperazin- 1-yl)pyridazin-3-yl)-4- fluoroisoindolin-1-one |
| 644 | 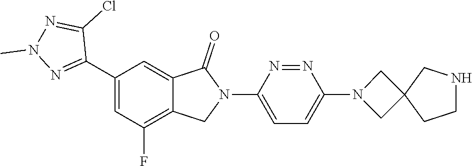 [SMILES: Cn1nc(Cl)c(-c2cc(F)c3c(c2)C(=O)N(c2ccc(N4CC5(CCNC5)C4)nn2)C3)n1] | 2-(6-(2,6-diazaspiro[3.4]octan-2- yl)pyridazin-3-yl)-6-(5-chloro-2- methyl-2H-1,2,3-triazol-4-yl)-4- fluoroisoindolin-1-one |
| 645 | 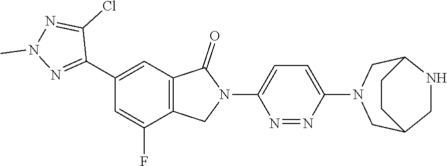 [SMILES: Cn1nc(Cl)c(-c2cc(F)c3c(c2)C(=O)N(c2ccc(N4CC5CCC(C4)NC5)nn2)C3)n1] | 2-(6-(2,5-diazabicyclo[2.2.2]octan- 2-yl)pyridazin-3-yl)-6-(5-chloro-2- methyl-2H-1,2,3-triazol-4-yl)-4- fluoroisoindolin-1-one |
| 646 | 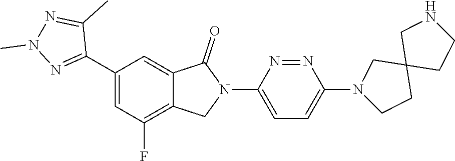 [SMILES: Cc1nn(C)nc1-c1cc(F)c2c(c1)C(=O)N(c1ccc(N3CCC4(CCNC4)C3)nn1)C2] | 2-(6-(2,7-diazaspiro[4.4]nonan-2- yl)pyridazin-3-yl)-6-(2,5-dimethyl- 2H-1,2,3-triazol-4-yl)-4- fluoroisoindolin-1-one |
| 647 | 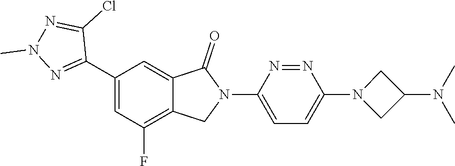 | 6-(5-chloro-2-methyl-2H-1,2,3- triazol-4-yl)-2-(6-(3- (dimethylamino)azetidin-1- yl)pyridazin-3-yl)-4- fluoroisoindolin-1-one |
| 648 | 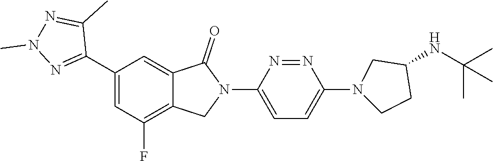 [SMILES: Cc1nn(C)nc1-c1cc(F)c2c(c1)C(=O)N(c1ccc(N3CC[C@@H](NC(C)(C)C)C3)nn1)C2] | (R)-2-(6-(3-(tert- butylamino)pyrrolidin-1- yl)pyridazin-3-yl)-6-(2,5-dimethyl- 2H-1,2,3-triazol-4-yl)-4- fluoroisoindolin-1-one |
| 650 | 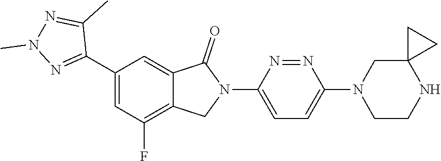 [SMILES: Cc1nn(C)nc1-c1cc(F)c2c(c1)C(=O)N(c1ccc(N3CCNC4(CC4)C3)nn1)C2] | 2-(6-(4,7-diazaspiro[2.5]octan-7- yl)pyridazin-3-yl)-6-(2,5-dimethyl- 2H-1,2,3-triazol-4-yl)-4- fluoroisoindolin-1-one |
| 651 | 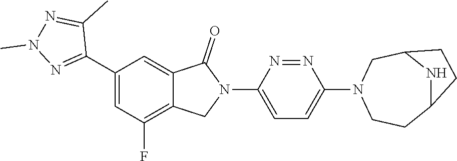 [SMILES: Cc1nn(C)nc1-c1cc(F)c2c(c1)C(=O)N(c1ccc(N3CCC4CCC(C3)N4)nn1)C2] | 2-(6-(3,9- diazabicyclo[4.2.1]nonan-3- yl)pyridazin-3-yl)-6-(2,5-dimethyl- 2H-1,2,3-triazol-4-yl)-4- fluoroisoindolin-1-one |
| 652 | 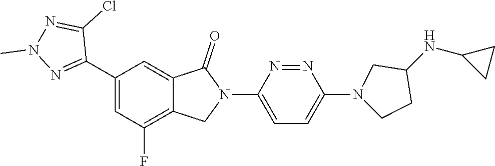 [SMILES: Cn1nc(Cl)c(-c2cc(F)c3c(c2)C(=O)N(c2ccc(N4CCC(NC5CC5)C4)nn2)C3)n1] | 6-(5-chloro-2-methyl-2H-1,2,3- triazol-4-yl)-2-(6-(3- (cyclopropylamino)pyrrolidin-1- yl)pyridazin-3-yl)-4- fluoroisoindolin-1-one |
| 653 | 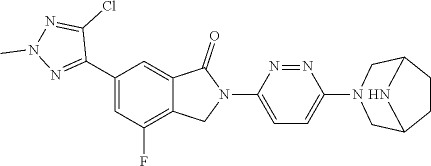 [SMILES: Cn1nc(Cl)c(-c2cc(F)c3c(c2)C(=O)N(c2ccc(N4CC5CCC(C4)N5)nn2)C3)n1] | 2-(6-(3,8-diazabicyclo[3.2.1]octan- 3-yl)pyridazin-3-yl)-6-(5-chloro-2- methyl-2H-1,2,3-triazol-4-yl)-4- fluoroisoindolin-1-one |
| 654 | 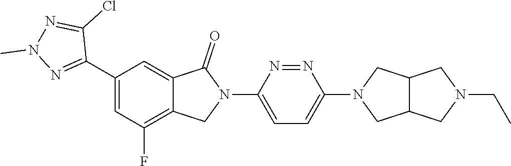 [SMILES: CCN1CC2CN(c3ccc(N4Cc5c(F)cc(-c6nn(C)nc6Cl)cc5C4=O)nn3)CC2C1] | 6-(5-chloro-2-methyl-2H-1,2,3- triazol-4-yl)-2-(6-(5- ethylhexahydropyrrolo[3,4- c]pyrrol-2(1H)-yl)pyridazin-3-yl)- 4-fluoroisoindolin-1-one |
| 655 | 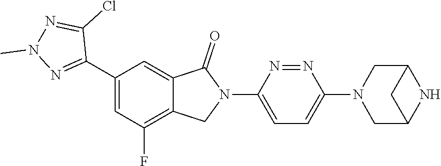 [SMILES: Cn1nc(Cl)c(-c2cc(F)c3c(c2)C(=O)N(c2ccc(N4CC5CC(C4)N5)nn2)C3)n1] | 2-(6-(3,6- diazabicyclo[3.1.1]heptan-3- yl)pyridazin-3-yl)-6-(5-chloro-2- methyl-2H-1,2,3-triazol-4-yl)-4- fluoroisoindolin-1-one |
| 656 | 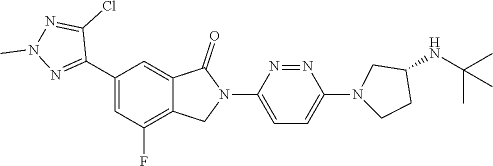 [SMILES: Cn1nc(Cl)c(-c2cc(F)c3c(c2)C(=O)N(c2ccc(N4CC[C@@H](NC(C)(C)C)C4)nn2)C3)n1] | (R)-2-(6-(3-(tert- butylamino)pyrrolidin-1- yl)pyridazin-3-yl)-6-(5-chloro-2- methyl-2H-1,2,3-triazol-4-yl)-4- fluoroisoindolin-1-one |
| 657 | 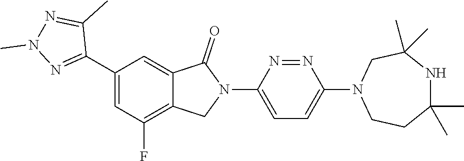 | 6-(2,5-dimethyl-2H-1,2,3-triazol- 4-yl)-4-fluoro-2-(6-(3,3,5,5- tetramethyl-1,4-diazepan-1- yl)pyridazin-3-yl)isoindolin-1-one |
| 658 | 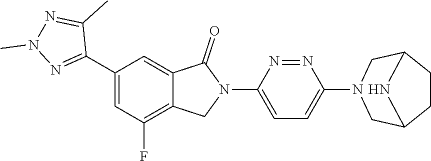 [SMILES: Cc1nn(C)nc1-c1cc(F)c2c(c1)C(=O)N(c1ccc(N3CC4CCC(C3)N4)nn1)C2] | 2-(6-(3,8-diazabicyclo[3.2.1]octan- 3-yl)pyridazin-3-yl)-6-(2,5- dimethyl-2H-1,2,3-triazol-4-yl)-4- fluoroisoindolin-1-one |
| 659 | 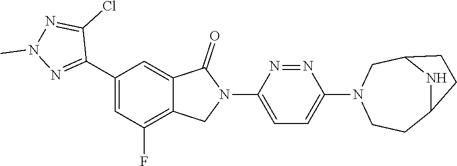 [SMILES: Cn1nc(Cl)c(-c2cc(F)c3c(c2)C(=O)N(c2ccc(N4CCC5CCC(C4)N5)nn2)C3)n1] | 2-(6-(3,9- diazabicyclo[4.2.1]nonan-3- yl)pyridazin-3-yl)-6-(5-chloro-2- methyl-2H-1,2,3-triazol-4-yl)-4- fluoroisoindolin-1-one |
| 662 | 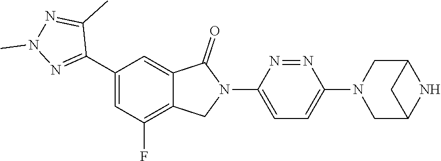 [SMILES: Cc1nn(C)nc1-c1cc(F)c2c(c1)C(=O)N(c1ccc(N3CC4CC(C3)N4)nn1)C2] | 2-(6-(3,6- diazabicyclo[3.1.1]heptan-3- yl)pyridazin-3-yl)-6-(2,5-dimethyl- 2H-1,2,3-triazol-4-yl)-4- fluoroisoindolin-1-one |
| 663 | 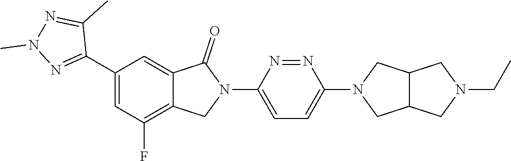 [SMILES: CCN1CC2CN(c3ccc(N4Cc5c(F)cc(-c6nn(C)nc6C)cc5C4=O)nn3)CC2C1] | 6-(2,5-dimethyl-2H-1,2,3-triazol- 4-yl)-2-(6-(5- ethylhexahydropyrrolo[3,4- c]pyrrol-2(1H)-yl)pyridazin-3-yl)- 4-fluoroisoindolin-1-one |
| 665 | 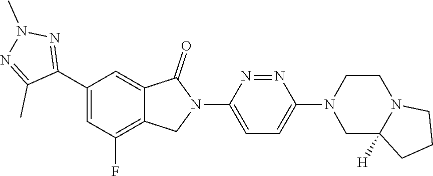 [SMILES: Cc1nn(C)nc1-c1cc(F)c2c(c1)C(=O)N(c1ccc(N3CCN4CCC[C@H]4C3)nn1)C2] | (S)-6-(2,5-dimethyl-2H-1,2,3- triazol-4-yl)-4-fluoro-2-(6- (hexahydropyrrolo[1,2-a]pyrazin- 2(1H)-yl)pyridazin-3- yl)isoindolin-1-one |
| 666 | 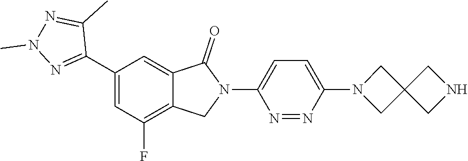 [SMILES: Cc1nn(C)nc1-c1cc(F)c2c(c1)C(=O)N(c1ccc(N3CC4(CNC4)C3)nn1)C2] | 2-(6-(2,6-diazaspiro[3.3]heptan-2- yl)pyridazin-3-yl)-6-(2,5-dimethyl- 2H-1,2,3-triazol-4-yl)-4- fluoroisoindolin-1-one |
| 667 | 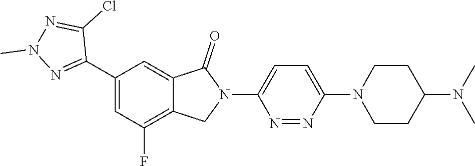 | 6-(5-chloro-2-methyl-2H-1,2,3- triazol-4-yl)-2-(6-(4- (dimethylamino)piperidin-1- yl)pyridazin-3-yl)-4- fluoroisoindolin-1-one |
| 668 | 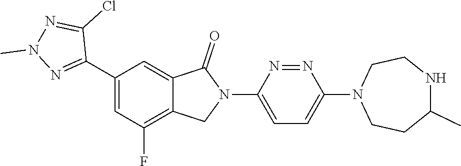 | 6-(5-chloro-2-methyl-2H-1,2,3- triazol-4-yl)-4-fluoro-2-(6-(5- methyl-1,4-diazepan-1- yl)pyridazin-3-yl)isoindolin-1-one |
| 669 | 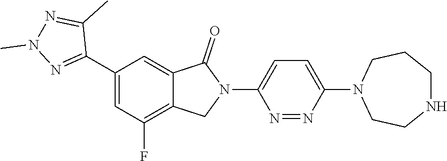 [SMILES: Cc1nn(C)nc1-c1cc(F)c2c(c1)C(=O)N(c1ccc(N3CCCNCC3)nn1)C2] | 2-(6-(1,4-diazepan-1-yl)pyridazin- 3-yl)-6-(2,5-dimethyl-2H-1,2,3- triazol-4-yl)-4-fluoroisoindolin-1- one |
| 670 | 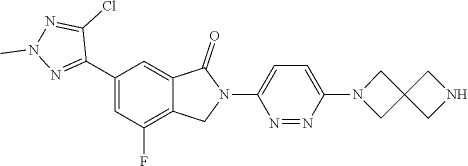 [SMILES: Cn1nc(Cl)c(-c2cc(F)c3c(c2)C(=O)N(c2ccc(N4CC5(CNC5)C4)nn2)C3)n1] | 2-(6-(2,6-diazaspiro[3.3]heptan-2- yl)pyridazin-3-yl)-6-(5-chloro-2- methyl-2H-1,2,3-triazol-4-yl)-4- fluoroisoindolin-1-one |
| 671 | 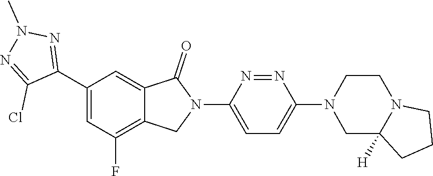 [SMILES: Cn1nc(Cl)c(-c2cc(F)c3c(c2)C(=O)N(c2ccc(N4CCN5CCC[C@H]5C4)nn2)C3)n1] | (S)-6-(5-chloro-2-methyl-2H- 1,2,3-triazol-4-yl)-4-fluoro-2-(6- (hexahydropyrrolo[1,2-a]pyrazin- 2(1H)-yl)pyridazin-3- yl)isoindolin-1-one |
| 672 | 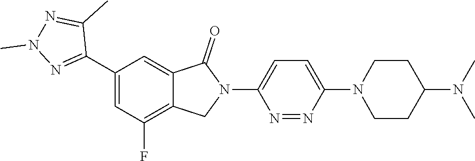 | 6-(2,5-dimethyl-2H-1,2,3-triazol- 4-yl)-2-(6-(4- (dimethylamino)piperidin-1- yl)pyridazin-3-yl)-4- fluoroisoindolin-1-one |
| 674 | 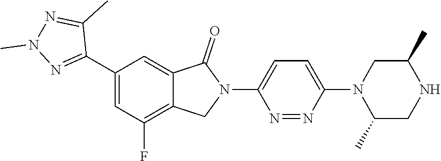 | 6-(2,5-dimethyl-2H-1,2,3-triazol- 4-yl)-2-(6-((2S,5R)-2,5- dimethylpiperazin-1-yl)pyridazin- 3-yl)-4-fluoroisoindolin-1-one |

In some embodiments the compound disclosed herein, or a pharmaceutically acceptable salt or solvate thereof, is one of the compounds Table 4. In some embodiments the compound disclosed herein, or a pharmaceutically acceptable salt or solvate thereof, is one of the compounds in Table 5.

TABLE 4
Exemplary agents that modulate splicing of MSH3 mRNA

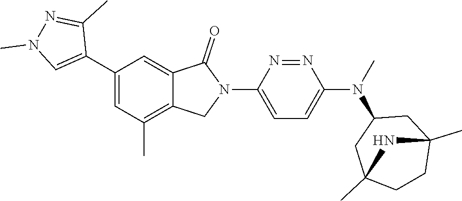

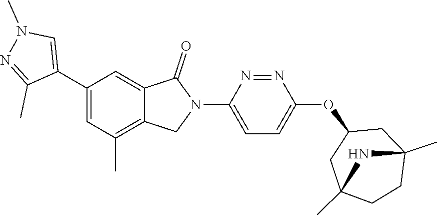 [SMILES: Cc1cc(-c2cn(C)nc2C)cc2c1CN(c1ccc(O[C@H]3C[C@]4(C)CC[C@](C)(C3)N4)nn1)C2=O]

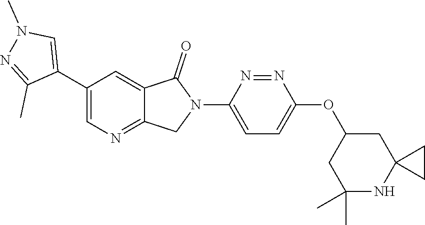 [SMILES: Cc1nn(C)cc1-c1cnc2c(c1)C(=O)N(c1ccc(OC3CC(C)(C)NC4(CC4)C3)nn1)C2]

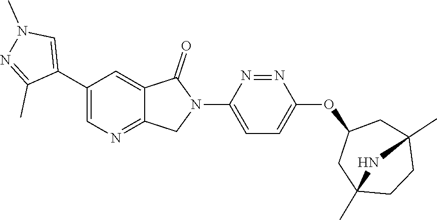 [SMILES: Cc1nn(C)cc1-c1cnc2c(c1)C(=O)N(c1ccc(O[C@H]3C[C@]4(C)CC[C@](C)(C3)N4)nn1)C2]

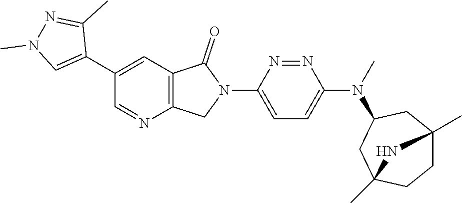

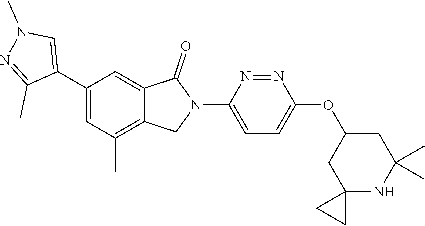 [SMILES: Cc1cc(-c2cn(C)nc2C)cc2c1CN(c1ccc(OC3CC(C)(C)NC4(CC4)C3)nn1)C2=O]

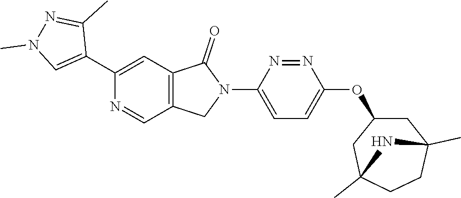

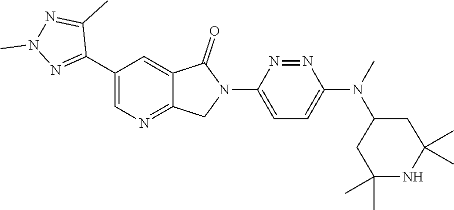

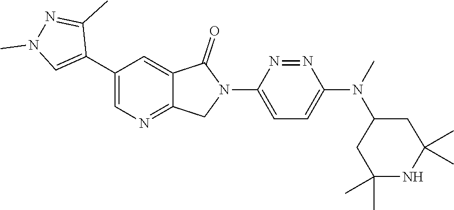

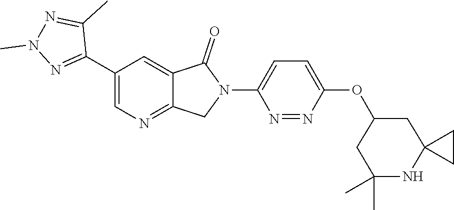 [SMILES: Cc1nn(C)nc1-c1cnc2c(c1)C(=O)N(c1ccc(OC3CC(C)(C)NC4(CC4)C3)nn1)C2]

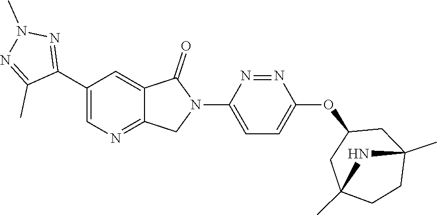 [SMILES: Cc1nn(C)nc1-c1cnc2c(c1)C(=O)N(c1ccc(O[C@H]3C[C@]4(C)CC[C@](C)(C3)N4)nn1)C2]

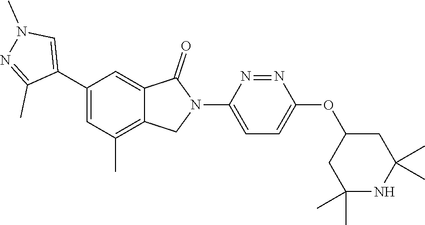 [SMILES: Cc1cc(-c2cn(C)nc2C)cc2c1CN(c1ccc(OC3CC(C)(C)NC(C)(C)C3)nn1)C2=O]

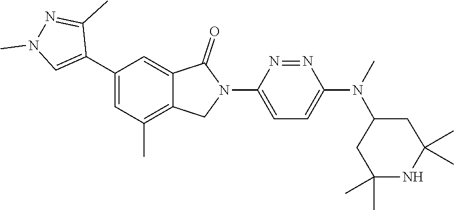

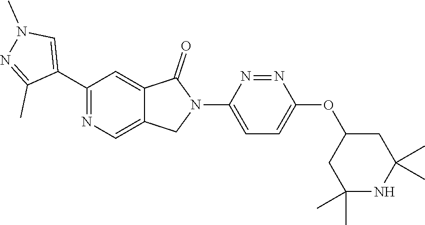 [SMILES: Cc1nn(C)cc1-c1cc2c(cn1)CN(c1ccc(OC3CC(C)(C)NC(C)(C)C3)nn1)C2=O]

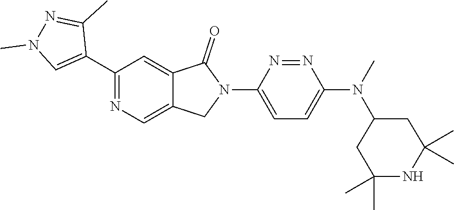

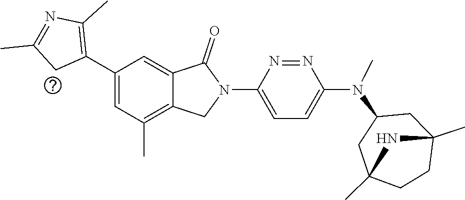

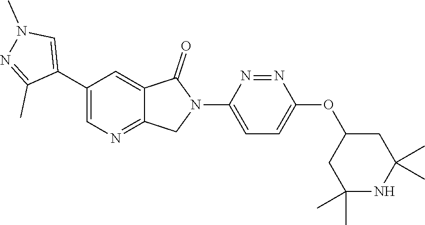 [SMILES: Cc1nn(C)cc1-c1cnc2c(c1)C(=O)N(c1ccc(OC3CC(C)(C)NC(C)(C)C3)nn1)C2]

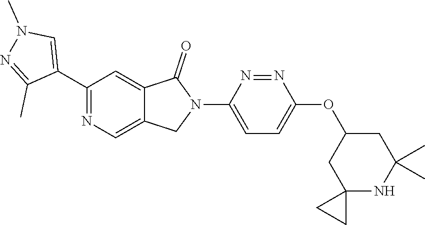 [SMILES: Cc1nn(C)cc1-c1cc2c(cn1)CN(c1ccc(OC3CC(C)(C)NC4(CC4)C3)nn1)C2=O]

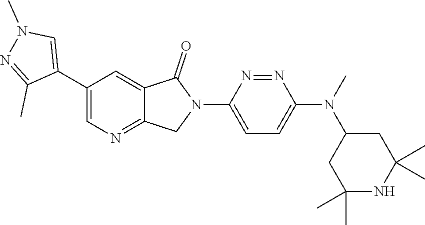 [SMILES: Cc1nn(C)cc1-c1cnc2c(c1)C(=O)N(c1ccc(N(C)C3CC(C)(C)NC(C)(C)C3)nn1)C2]

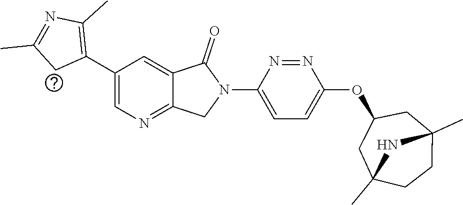

TABLE 5
Exemplary agents that modulate splicing of MSH3 and HTT mRNA

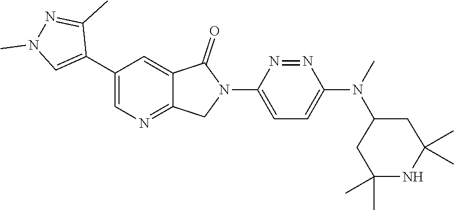

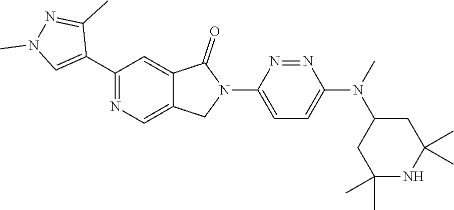

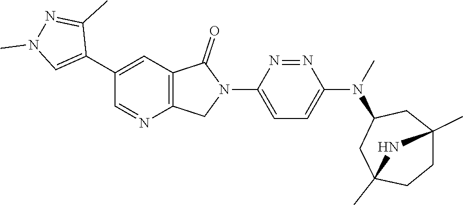

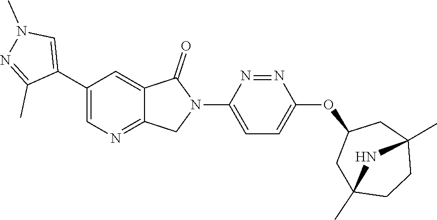 [SMILES: Cc1nn(C)cc1-c1cnc2c(c1)C(=O)N(c1ccc(O[C@H]3C[C@]4(C)CC[C@](C)(C3)N4)nn1)C2]

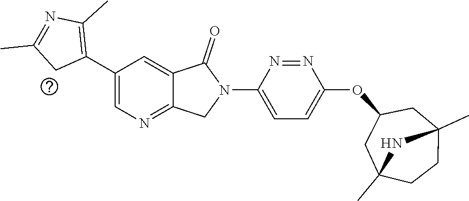

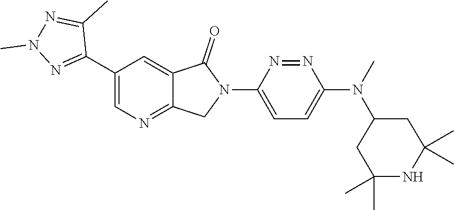

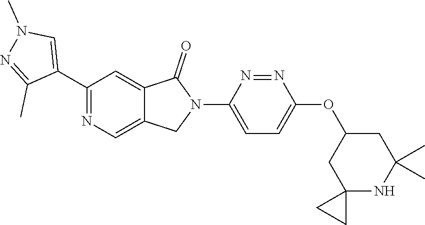 [SMILES: Cc1nn(C)cc1-c1cc2c(cn1)CN(c1ccc(OC3CC(C)(C)NC4(CC4)C3)nn1)C2=O]

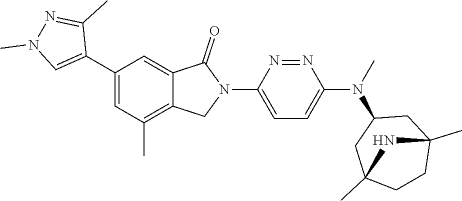

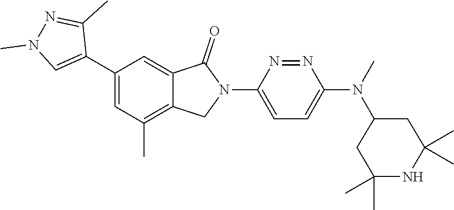

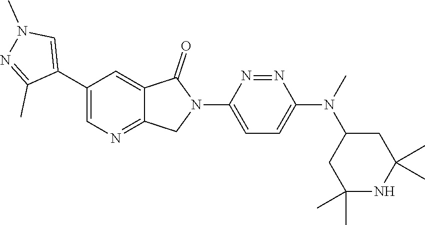 [SMILES: Cc1nn(C)cc1-c1cnc2c(c1)C(=O)N(c1ccc(N(C)C3CC(C)(C)NC(C)(C)C3)nn1)C2]

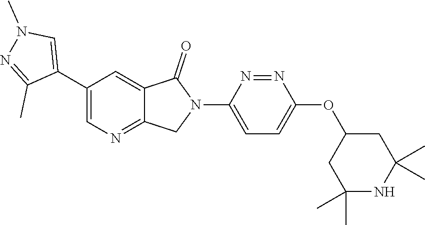 [SMILES: Cc1nn(C)cc1-c1cnc2c(c1)C(=O)N(c1ccc(OC3CC(C)(C)NC(C)(C)C3)nn1)C2]

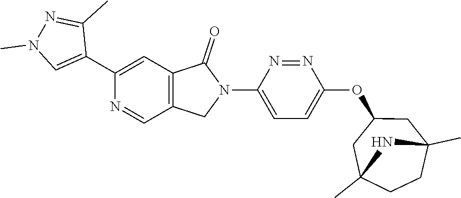

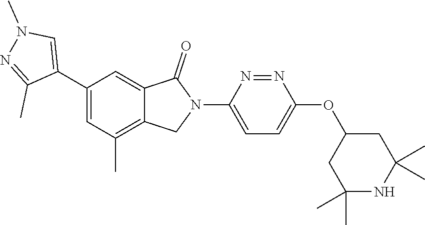 [SMILES: Cc1cc(-c2cn(C)nc2C)cc2c1CN(c1ccc(OC3CC(C)(C)NC(C)(C)C3)nn1)C2=O]

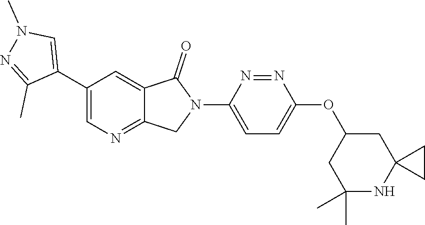 [SMILES: Cc1nn(C)cc1-c1cnc2c(c1)C(=O)N(c1ccc(OC3CC(C)(C)NC4(CC4)C3)nn1)C2]

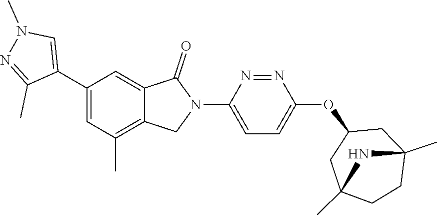 [SMILES: Cc1cc(-c2cn(C)nc2C)cc2c1CN(c1ccc(O[C@H]3C[C@]4(C)CC[C@](C)(C3)N4)nn1)C2=O]

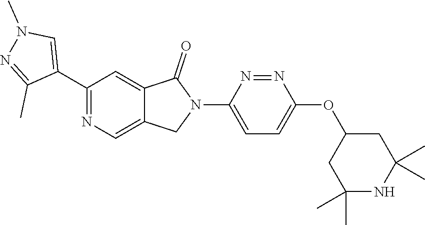 [SMILES: Cc1nn(C)cc1-c1cc2c(cn1)CN(c1ccc(OC3CC(C)(C)NC(C)(C)C3)nn1)C2=O]

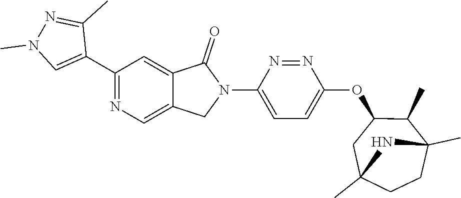

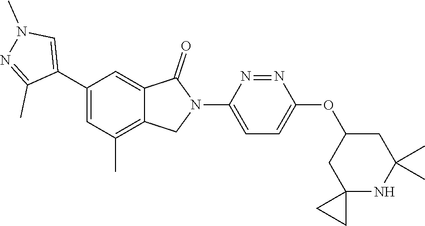 [SMILES: Cc1cc(-c2cn(C)nc2C)cc2c1CN(c1ccc(OC3CC(C)(C)NC4(CC4)C3)nn1)C2=O]

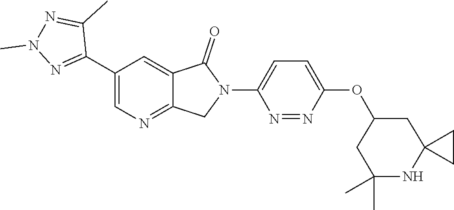 [SMILES: Cc1nn(C)nc1-c1cnc2c(c1)C(=O)N(c1ccc(OC3CC(C)(C)NC4(CC4)C3)nn1)C2]

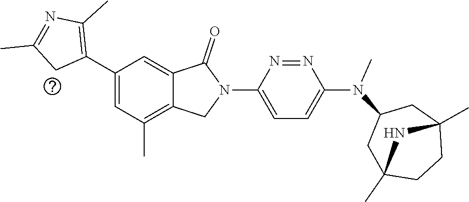

In some embodiments, the agent is a compound selected from the group consisting of:

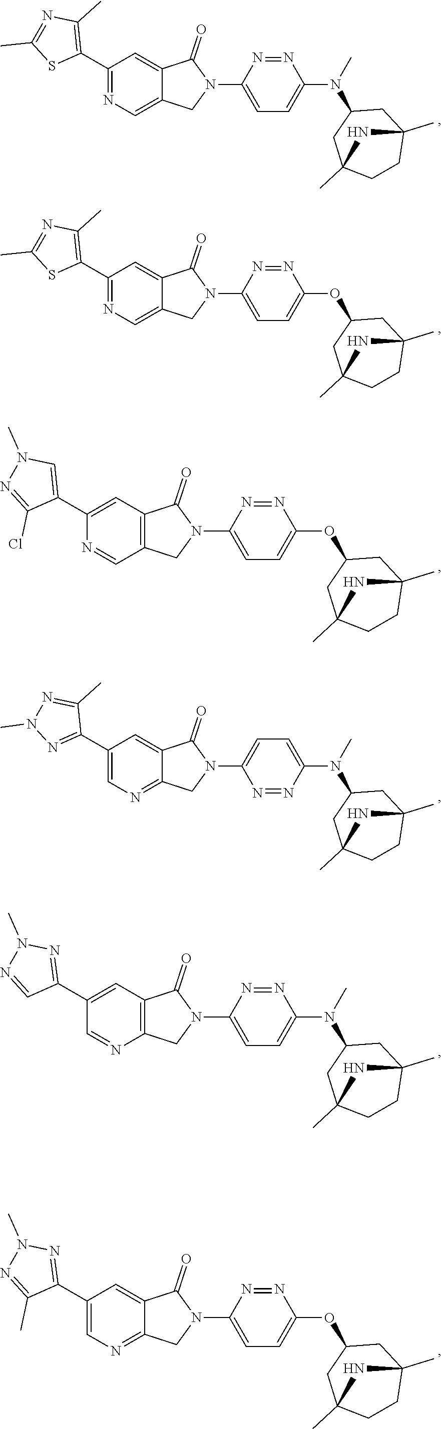

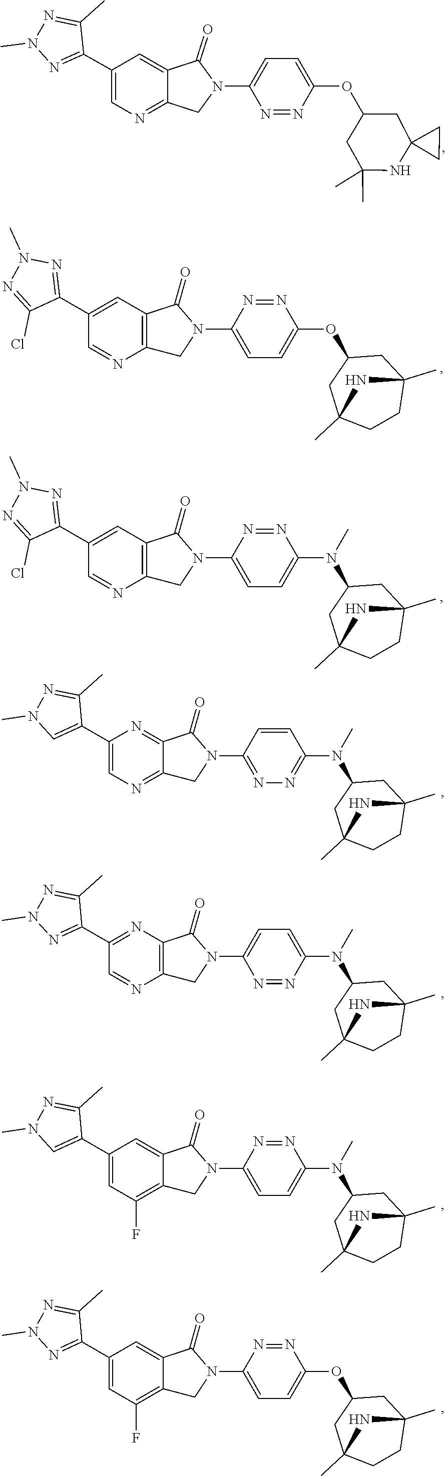

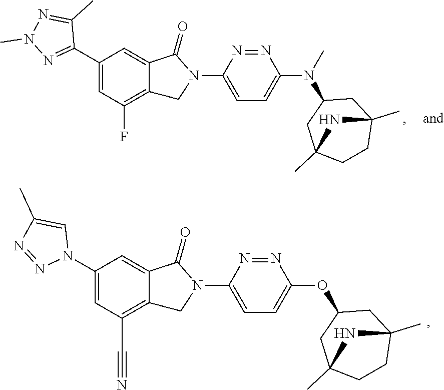

or a pharmaceutically acceptable salt or solvate thereof.

In some embodiments, the agent is a compound that modulates splicing of the MSH3 pre-mRNA such that a poison exon is spliced into the MSH3 mRNA transcript. In some cases, the agent that modulates splicing of the MSH3 pre-mRNA such that a poison exon is spliced into the MSH3 mRNA transcript is selected from any compound from Table 3A, Table 3B, or Table 3C. In some cases, the agent that modulates splicing of the MSH3 pre-mRNA such that a poison exon is spliced into the MSH3 mRNA transcript is selected from any compound from Table 4. In some cases, the agent that modulates splicing of the MSH3 pre-mRNA such that a poison exon is spliced into the MSH3 mRNA transcript is selected from any compound from Table 5.

In some cases, the agent is an agent that modulates splicing of the MSH3 pre-mRNA as described herein and does not modulate splicing of other genes. In some cases, the agent that modulates splicing of the MSH3 pre-mRNA as described herein and does not modulate splicing of other genes is selected from Table 4.

In some cases, the agent modulates splicing of the MSH3 pre-mRNA as described herein, and also modulates splicing of at least one other gene. Advantageously, in such scenarios, a single agent species can be used to modulate splicing of more than one gene. In an exemplary instance, the agent can modulate splicing of MSH3 pre-mRNA as described herein, and can modulate splicing of HTT pre-mRNA. In some cases, the agent that modulates splicing of both MSH3 pre-mRNA and HTT pre-mRNA is selected from Table 5.

In some embodiments, reducing expression of MSH3 (such as by the compositions, methods, and systems provided herein), results in decreased nucleotide repeat expansion of a target gene in a cell. In some embodiments, the nucleotide repeat expansion comprises a trinucleotide repeat expansion. In some embodiments, the nucleotide repeat expansion comprises expansion of a four-nucleotide repeat, a five-nucleotide repeat, a six-nucleotide repeat, a seven-nucleotide repeat, an eight-nucleotide repeat, a nine-nucleotide repeat, a ten-nucleotide repeat, an eleven-nucleotide repeat, or a twelve-nucleotide repeat. In some embodiments, the nucleotide repeat expansion is in an exon of the target gene. In some embodiments, the nucleotide repeat expansion is in an intron of the target gene. In some embodiments, the nucleotide repeat expansion is in a UTR of the target gene. In some embodiments, the nucleotide repeat expansion is in a promoter of the target gene. In some embodiments, the nucleotide repeat expansion in the cell comprises at least 20 repeats, at least 30 repeats, at least 40 repeats, at least 50 repeats, at least 60 repeats, at least 70 repeats, at least 80 repeats, at least 90 repeats, at least 100 repeats, at least 200 repeats, at least 500 repeats, at least 1000 repeats, at least 2000 repeats, at least 5000 repeats, at least 10,000 repeats, or at least 20,000 repeats. In some embodiments, the method decreases nucleotide repeat expansion in the target gene by at least about 0.1 fold, at least about 0.2 fold, at least about 0.5 fold, at least about 1.0 fold, at least about 2.0 fold, at least about 5.0 fold, at least about 10.0 fold, or at least about 100.0 fold, as compared to a level of nucleotide repeat expansion in the target gene in the absence of the agent.

In some cases, the target gene is selected from the group consisting of: ATN1, HTT, AR, ATXN1, ATXN2, ATXN3, CACNA1A, ATXN7, PPP2R2B, TBP, TCF4, JPH3, DMPK, ATXN8, ZNF713, FMR1, AFF/FMR2, FXN, ZFN9, ATXN10, NOP56, C₉orf72, CSTB, and any combination thereof. In some embodiments, the target gene is associated with a nucleotide repeat expansion disease or condition. In some embodiments, the nucleotide repeat expansion disease or condition is selected from the group consisting of: dentatorubropallidoluysian atrophy (DRPLA), Huntington's disease (HD), spinal and bulbar muscular atrophy (SBMA), spinocerebellar ataxia (SCA) type 1, spinocerebellar ataxia (SCA) type 2, spinocerebellar ataxia (SCA) type 3, spinocerebellar ataxia (SCA) type 6, spinocerebellar ataxia (SCA) type 7, spinocerebellar ataxia (SCA) type 8, spinocerebellar ataxia (SCA) type 10, spinocerebellar ataxia (SCA) type 12, spinocerebellar ataxia (SCA) type 17, spinocerebellar ataxia (SCA) type 31, spinocerebellar ataxia (SCA) type 36, spinocerebellar ataxia (SCA) type 37, spinocerebellar ataxia (SCA) type 50, Friedreich ataxia, fragile X syndrome (FRAXA), fragile X-associated tremor/ataxia syndrome (FXTAS), Baratela-Scott syndrome (BSS), myotonic dystrophy (DM) type 1, myotonic dystrophy (DM) type 2, amyotrophic lateral sclerosis (ALS), Kennedy's disease, fragile X E mild mental retardation (FRAXE), progressive myoclonus epilepsy (EPM1), oculopharyngodistal myopathy (OPDM) type 1, oculopharyngodistal myopathy (OPDM) type 2, oculopharyngeal myopathy with leukoencephalopathy (OPML), frontotemporal dementia (FTD), Neuronal intranuclear inclusion disease (NIID), essential tremor, global developmental delay, progressive ataxia, and elevated glutamine (GDPAG), familial myoclonic epilepsy (FAME) type 1, familial myoclonic epilepsy (FAME) type 2, familial myoclonic epilepsy (FAME) type 3, familial myoclonic epilepsy (FAME) type 4, familial myoclonic epilepsy (FAME) type 6, familial myoclonic epilepsy (FAME) type 7, premature ovarian failure (FXPOF), cerebellar ataxia, neuropathy, vestibular areflexia syndrome (CANVAS), multiple system atrophy (MSA), refractory chronic cough, Fuchs endothelial corneal dystrophy (FECD), X-linked dystonia-parkinsonism (XDP), intellectual disability (FRA12A type), Jacobsen syndrome (CBL2), and any combination thereof. Accordingly, the compositions, methods, and systems may be used to treat any one of the nucleotide repeat expansion diseases or conditions provided herein. Table 6 illustrates non-limiting examples of diseases or conditions associated with a gene having a nucleotide repeat expansion (e.g., that can be treated by the compositions, methods, and systems provided herein).

TABLE 6
Nucleotide repeat expansions diseases or conditions

| | | Nucleotide |
|---|---|---|
| Nucleotide repeat expansion disease or condition | Gene | repeat |
| Fragile X E mild mental retardation (FRAXE) | AFF2 | CCG |
| Dentatorubral pallidoluysian atrophy (DRPLA) | ATN1 | CAG |
| Huntington's Disease (HD) | HTT | CAG |
| Huntington's Disease (HD) | HDL2 | CTG |
| Progressive myoclonus epilepsy (EPM1) | CSTB | CCCCGCCCCG |
| | | CG (SEQ ID |
| | | NO: 13) |
| Myotonic dystrophy (DM) type 1 | DMPK | CTG |
| Myotonic dystrophy (DM) type 2 | CNBP | CCTG |
| Oculopharyngodistal myopathy (OPDM) type 1 | LR[12 | CGG |
| Oculopharyngodistal myopathy (OPDM) type 2 | GIPC1 | CGG |
| Oculopharyngeal myopathy with leukoencephalopathy | NUTM2B- | CGG |
| (OPML) | AS1 | |
| Frontotemporal dementia (FTD) | C9orf72 | GGGGCC |
| Amyotrophic lateral sclerosis (ALS) | C9orf72 | GGGGCC |
| Neuronal intranuclear inclusion disease (NIID) and | NOTCH2NLC | CGG |
| essential tremor | | |
| Global developmental delay, progressive ataxia, and | GLS | CGA |
| elevated glutamine (GDPAG) | | |
| Spinocerebellar ataxia (SCA) type 1 | ATXN1 | CAG |
| Spinocerebellar ataxia (SCA) type 2 | ATXN2 | CAG |
| Spinocerebellar ataxia (SCA) type 3 | ATXN3 | CAG |
| Spinocerebellar ataxia (SCA) type 6 | CACNA1A | CAG |
| Spinocerebellar ataxia (SCA) type 7 | ATXN7 | CAG |
| Spinocerebellar ataxia (SCA) type 12 | PPP2R2B | CAG |
| Spinocerebellar ataxia (SCA) type 17 | TBP | CAG |
| Familial myoclonic epilepsy (FAME) type 1 | SAMD12 | ATTTC |
| Familial myoclonic epilepsy (FAME) type 2 | STARD7 | ATTTC |
| Familial myoclonic epilepsy (FAME) type 3 | MARCHF6 | ATTTC |
| Familial myoclonic epilepsy (FAME) type 4 | YEATS2 | ATTTC |
| Familial myoclonic epilepsy (FAME) type 6 | TNRC6A | ATTTC |
| Familial myoclonic epilepsy (FAME) type 7 | RAPGEF2 | ATTTC |
| Fragile X syndrome (FRAXA) | FMR1 | CGG |
| Fragile X-associated tremor ataxia syndrome (FXTAS) | FMR1 | CGG |
| Premature ovarian failure (FXPOF) | FMR1 | CGG |
| Cerebellar ataxia, neuropathy, vestibular areflexia | RFC1 | AAGGG |
| syndrome (CANVAS) | | |
| Multiple system atrophy (MSA) | RFC2 | AAGGG |
| Refractory chronic cough | RFC3 | AAGGG |
| Spinocerebellar ataxia (SCA) type 8 | ATXN8OS | CTG |
| Spinocerebellar ataxia (SCA) type 10 | ATXN10 | ATTCT |
| Spinocerebellar ataxia (SCA) type 31 | TK2/BEAN1 | TGGAA |
| Spinocerebellar ataxia (SCA) type 36 | NOP56 | GGCCTG |
| Spinocerebellar ataxia (SCA) type 37 | DAB1 | ATTTC |
| Spinocerebellar ataxia (SCA) type 50 | FGF14 | GAA |
| Fuchs endothelial corneal dystrophy (FECD) | TCF4 | CTG |
| X-linked dystonia-parkinsonism (XDP) | TAF1 | CCCTCT |
| Spinal and bulbar muscular atrophy (SBMA, or Kennedy's | AR | CAG |
| disease) | | |
| Baratela-Scott syndrome (BSS) | XYLT1 | GGC |
| Intellectual disability, FRA12A type | DIP2B | CGG |
| Jacobsen syndrome (CBL2) | CBL2 | CGG |
| Friedreich ataxia (FA) | FXN | GAA |

Further provided herein, in some aspects, is a method for screening for an agent that modulates splicing of an MSH3 pre-nmRNA. In some embodiments, the method comprises: contacting a cell with a candidate agent; detecting presence or absence of a poison exon in an MSH3 mRNA transcript; and identifying the candidate agent as an agent that modulates splicing of the MSH3 pre-mRNA when the poison exon is detected in the MSH3 mRNA transcript. In some embodiments, the detecting comprises sequencing the MSH3 mRNA transcript. In some embodiments, the detecting comprises detecting an amount of the MSH3 mRNA transcript comprising the poison exon. In some embodiments, the detecting comprises determining a level of MSH3 protein expression. In some cases, the identifying comprises identifying the candidate agent as an agent that modulates splicing of the MSH3 pre-mRNA when the level of MSH3 protein expression is decreased as compared to a level of MSH3 protein expression in the absence of the candidate agent. In some embodiments, the detecting comprises determining or measuring a level of MSH3 mRNA transcript. In some embodiments, the method comprises identifying the candidate agent as an agent that modulates splicing of the MSH3 pre-mRNA when the level of MSH3 mRNA transcript is decreased as compared to a level of MSH3 mRNA transcript in the absence of the candidate agent. In some embodiments, the method comprises identifying the candidate agent as an agent that modulates splicing of the MSH3 pre-mRNA and the pre-mRNA of at least one additional gene. In some embodiments, the detecting comprises detecting viability of the cell. In some embodiments, the identifying comprises identifying the candidate agent as an agent that modulates splicing of the MSH3 pre-mRNA when viability of the cell is decreased as compared to in the absence of the candidate agent. In some embodiments, the method further comprises contacting the cell with an inhibitor of nonsense-mediated decay (NMD). In some cases, the inhibitor of NMD is cycloheximide. In some embodiments, the method further comprises determining that the poison exon leads to NMD of the MSH3 protein when a level of MSH3 protein expression is decreased in the absence of the inhibitor of NMD, and a level of MSH3 protein expression is increased in the presence of the inhibitor of NMD. In some embodiments, the method further comprises contacting a plurality of cells with a library of candidate agents, wherein each cell of the plurality of cells is contacted with a different candidate agent.

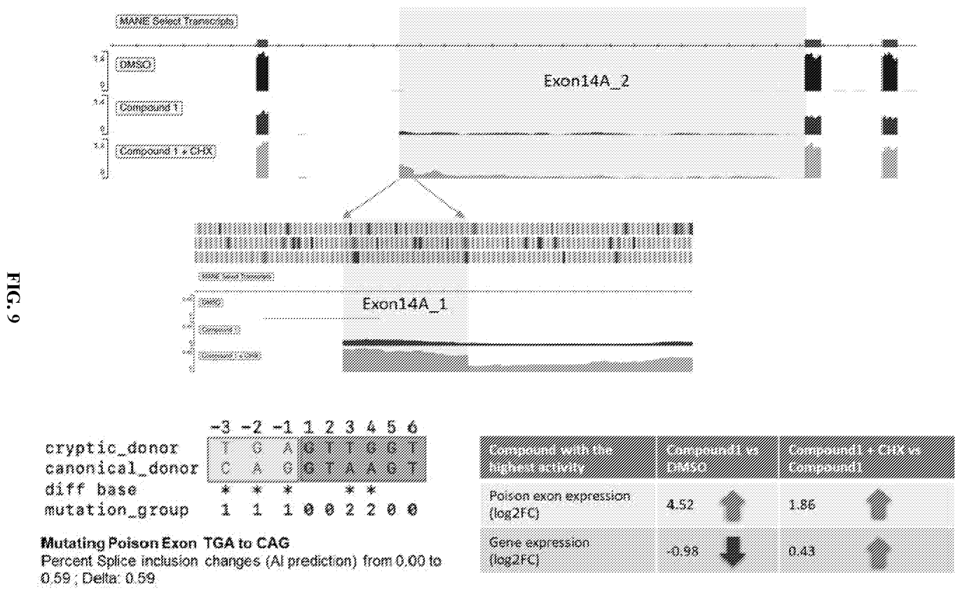

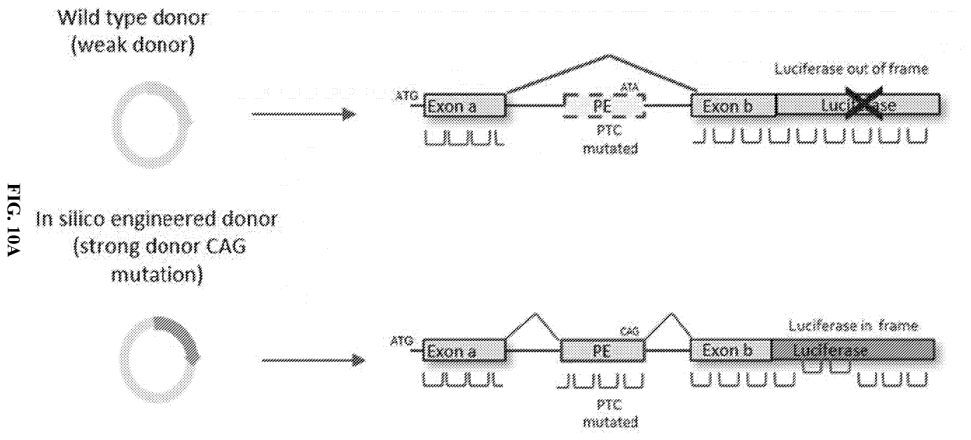

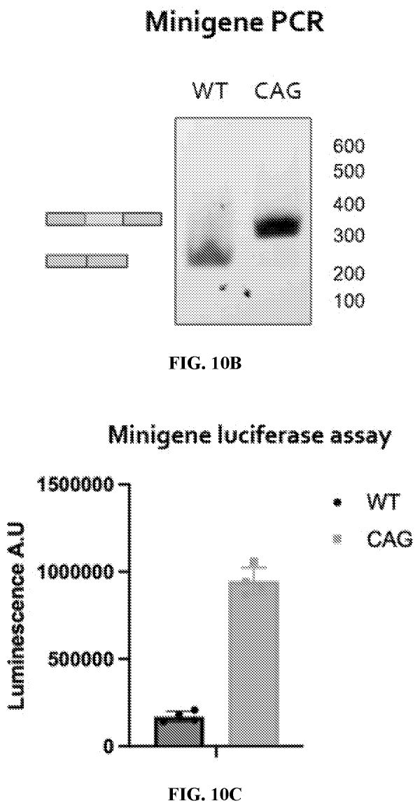

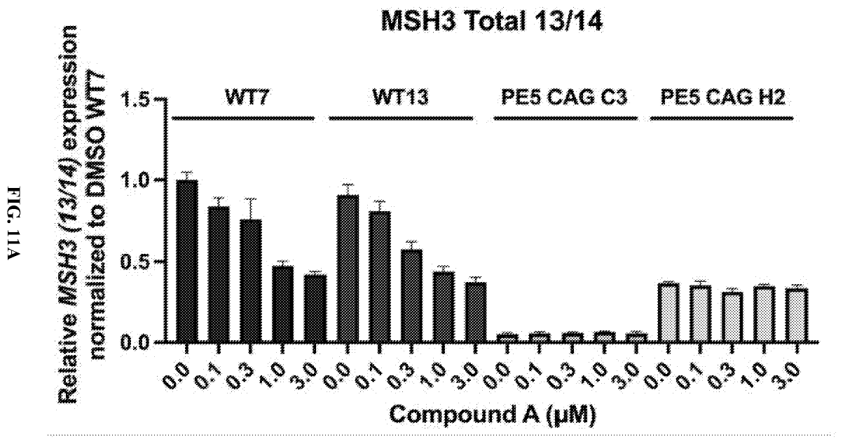

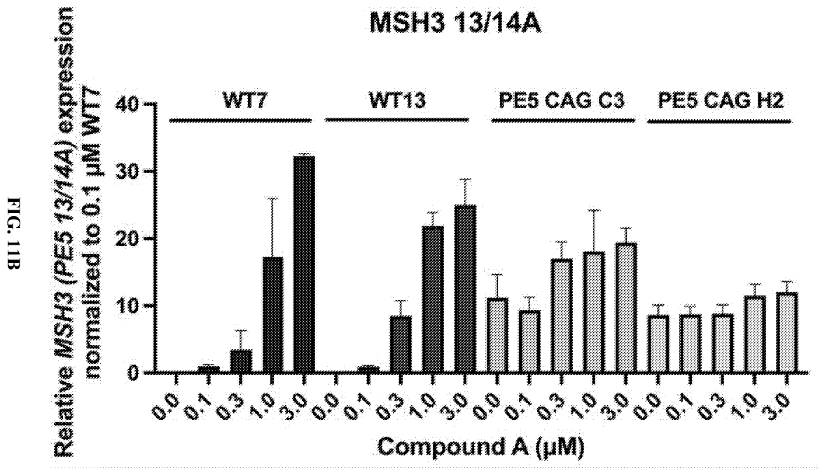

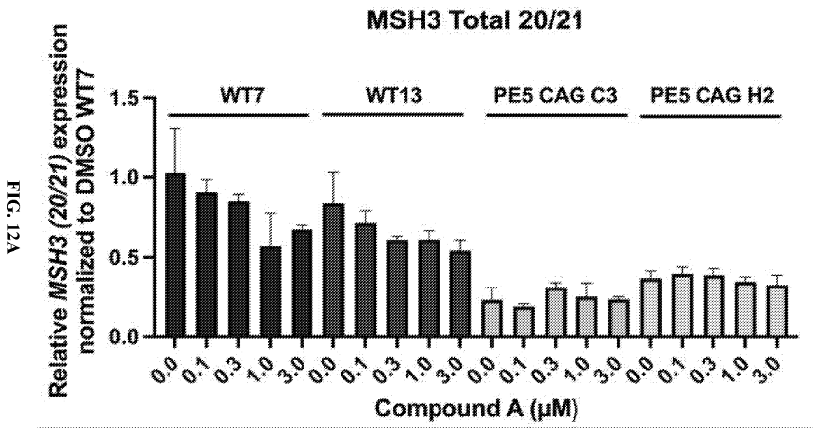

In some embodiments, the method comprises screening for candidate agents by contacting the candidate agents with a poison exon comprising nucleic acid mutation for enhancing an output associated with the contacting of the candidate agents and the poison axon comprising the nucleic acid mutation. As shown in FIG. 10A, an engineered minigene donor (e.g., by mutating endogenous trinucleotides to CAG as shown in FIG. 8 and FIG. 9) can drive expression of a luciferase reporter gene. FIG. 10B illustrates minigene PCR showing the presence of the poison exon comprising the CAG mutation. The wild-type (WT) mRNA did not have the poison exon as confirmed by the decreased nucleotide length. FIG. 10C illustrates increased luciferase signal generated by the presence of the minigene comprising the poison exon comprising the CAG mutation. In such case, the output can be the luciferase signal. In some embodiments, the output comprises RNASeq readout. In some embodiments, the output comprises detecting cell viability of a cell comprising the MSH3 pre-mRNA comprising the poison exon and the spliceosomal RNA. In some embodiments, output comprises a reporter signal, said reporter signal is encoded by the MSH3 pre-mRNA comprising the poison exon.

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (R), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine, and chloride, such as ²H (D), ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability.

In some embodiments, the abundance of deuterium in each of the substituents disclosed herein is independently at least 1%, at least 10%, at least 20%, at least 30%, at least 40%, at least 50%, at least 60%, at least 70%, at least 80%, at least 90%, or 100% by molar. In some embodiments, one or more of the substituents disclosed herein comprise deuterium at a percentage higher than the natural abundance of deuterium. In some embodiments, one or more ¹H are replaced with one or more deuteriums in one or more of the substituents disclosed herein.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or a solvate, or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein, solvate, or stereoisomer thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. In some embodiments, the disclosure provides for methods of treating diseases by administering the compounds in the form of such solvates. In some embodiments, the disclosure provides for methods of treating diseases by administering a composition comprising the compounds in the form of such solvates. Solvates contain either stoichiometric or non-stoichiometric amounts of a solvent, and, in some embodiments, are formed during the process of crystallization with pharmaceutically acceptable solvents.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Dosing

In certain embodiments, the compositions containing the compound(s) described herein are administered for therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, in specific embodiments, the dosage, or the frequency of administration, or both, is reduced, as a function of the symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In some embodiments, doses employed for adult human treatment are typically in the range of 0.01 mg-5000 mg per day. In some embodiments, the daily dosages appropriate for the compound described herein, or a pharmaceutically acceptable salt thereof, are from about 0.01 to about 50 mg/kg per body weight. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation.

### Pharmaceutical Compositions/Formulations

The compounds described herein are administered to a subject in need thereof, either alone or in combination with pharmaceutically acceptable carriers, excipients, or diluents, in a pharmaceutical composition, according to standard pharmaceutical practice. In some embodiments, the compounds described herein are administered to animals.

In another aspect, provided herein are pharmaceutical compositions comprising a compound described herein, or a pharmaceutically acceptable salt thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, (N.Y., 1980; and Pharmaceutical Dosage Forms and Drug Delivery Systems, Seventh Ed. (Lippincott Williams & Wilkins 1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

### EXAMPLES

The following examples are offered to illustrate, but not to limit the claimed invention. The following examples further illustrate the invention but, of course, should not be construed as in any way limiting its scope.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

The compounds and salts of Formulas (I), (II), (III), (IIIa), (IIIb), and (IV) can be synthesized according to one or more illustrative schemes herein and/or techniques known in the art. Materials used herein are either commercially available or prepared by synthetic methods generally known in the art. These schemes are not limited to the compounds listed in the examples or by any particular substituents, which are employed for illustrative purposes. Although various steps are described and depicted in the synthesis schemes below, the steps in some cases may be performed in a different order than the order shown below. Numberings or R groups in each scheme do not necessarily correspond to that of the claims or other schemes or tables herein.

### Abbreviations

AcOH: Acetic acid
Boc₂O: Di-tert-butyl dicarbonate
MeCN or ACN: Acetonitrile
DavePhos: 2-Dicyclohexylphosphino-2′-(N,N-dimethylamino)biphenyl
DCC: N,N′-Dicyclohexylcarbodiimide
DCM: Dichloromethane
DDQ: 2,3-Dichloro-5,6-dicyano-1,4-benzoquinone
Dioxane: 1,4-Dioxane
DIPEA: N, N-Diisopropylethylamine
DMF Dimethylformamide
DMSO Dimethyl sulfoxide
EDCl: N-(3-Dimethylaminopropyl)-N′-ethylcarbodiimide hydrochloride
eq Equivalents
EtOAc Ethyl acetate
Et₃N: Triethylamine
g Grams
h Hour or hours
HATU: Hexafluorophosphate Azabenzotriazole Tetramethyl Uronium
HOBT: 1-Hydroxybenzotriazole
HOSu: N-hydroxysuccinimide
LCMS: Liquid chromatography-mass spectrometry
LAH: Lithium aluminum hydride
MeOH: Methanol
mg Milligrams
min Minute or minutes
mL Milliliters
mmol Millimoles
MS Mass spectrometry
MTBE: Methyl tertiary-butyl ether
NCS: N-Chlorosuccinimide
NMR: Nuclear Magnetic Resonance
PCC: Pyridinium chlorochromate
Pd: Palladium
Pd₂(dba)₃: Tris(dibenzylideneacetone)dipalladium
Pd(dppf)Cl₂: [1,1′-Bis(diphenylphosphino)ferrocene]palladium(II) dichloride
RP-HPLC: Reversed-Phase High-Performance Liquid Chromatography
rt or RT: Room temperature, ambient, about 25° C.
THF: Tetrahydrofuran
TLC: Thin-layer chromatography

### Example 1. Synthesis of 2-(6-(((1S,3R,5R)-2,2-difluoro-1,5-dimethyl-8-azabicyclo[3.2.1]octan-3-yl)oxy)pyridazin-3-yl)-6-(1,3-dimethyl-1H-pyrazol-4-yl)-2,3-dihydro-1H-pyrrolo[3,4-c]pyridin-1-one (Compound No. 82)

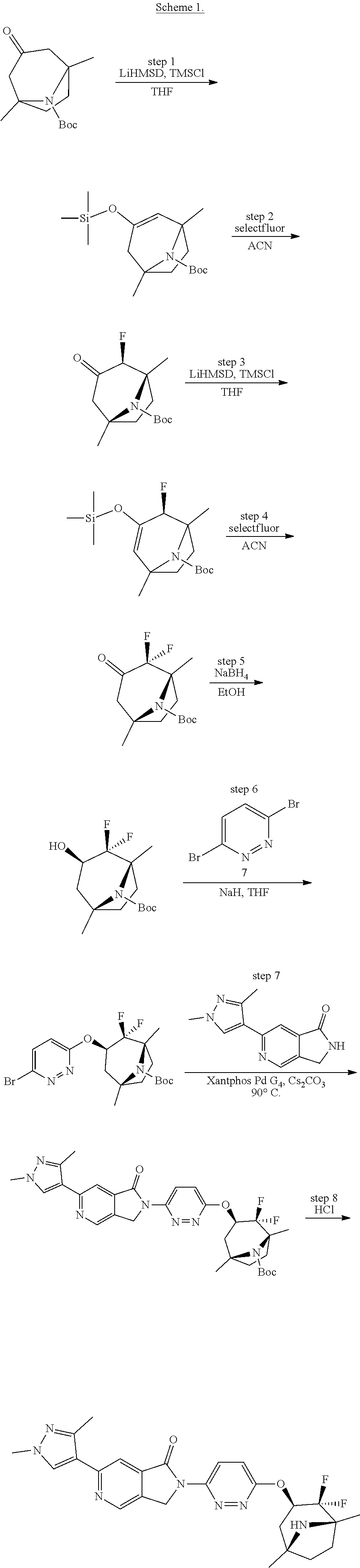

Step 1: To a solution of 2-methylpropan-2-yl 1,5-dimethyl-3-oxo-8-azabicyclo[3.2.1]octane-8-carboxylate (2.50 g, 9.87 mmol) in THF (30 mL) was added LiHMDS (15.8 mL, 15.8 mmol) at −65° C. The reaction was stirred for 1.5 h and then TMSCl (1.6 mL, 12.83 mmol) was added and the reaction mixture stirring at −78° C. for another 2 h. After the reactants were consumed, the reaction was diluted with EA and H₂O. The organic layer was separated, washed with further saturated NaCl solution, and concentrated in vacuo to afford 2-methylpropan-2-yl 1,5-dimethyl-3-[(trimethylsilyl)oxy]-8-azabicyclo[3.2.1]oct-2-ene-8-carboxylate (3.0 g, 93.4%) as a colorless oil which was used directly in the next step without further purification. MS (ESI, pos. ion) m/z 326.5 [M+H]⁺.

Step 2: To a solution of 2-methylpropan-2-yl 1,5-dimethyl-3-[(trimethylsilyl)oxy]-8-azabicyclo[3.2.1]oct-2-ene-8-carboxylate (3.0 g, 9.22 mmol) in acetonitrile (15 mL) was added Selectfluor (5.24 g, 14.8 mmol) and the reaction mixture was stirred at room temperature overnight. After the reactants were consumed, the reaction was quenched with water and then diluted with EA (100 mL). The organic layer was collected, dried over sodium sulfate, and concentrated in vacuo. The crude mixture was purified by silica gel column chromatography with DCM/MeOH=20/1, to provided 2-methylpropan-2-yl (1R,4S,5S)-4-fluoro-1,5-dimethyl-3-oxo-8-azabicyclo[3.2.1]octane-8-carboxylate (1.8 g, 71.98%) as a white solid. MS (ESI, pos. ion) m/z 272.3 [M+H]⁺.

Step 3: 2-Methylpropan-2-yl (1R,4S,5S)-4-fluoro-1,5-dimethyl-3-oxo-8-azabicyclo[3.2.1]octane-8-carboxylate (1.3 g, 4.79 mmol) was dissolved in THF (30 mL) and LiHMDS (7.67 mL, 7.67 mmol) was added at 80° C. The reaction was stirred at −65° C. for 1.5 h, and then TMSCl (0.79 mL, 6.23 mmol) was added. The reaction mixture was stirred for another 2 h at −78° C. After the reactants were consumed, the reaction was diluted with EA and H₂O. The organic layer was separated, washed with further saturated NaCl solution, and concentrated in vacuo to afford 2-methylpropan-2-yl (1S,5R)-2-fluoro-1,5-dimethyl-3-[(trimethylsilyl)oxy]-8-azabicyclo[3.2.1]oct-2-ene-8-carboxylate (1.6 g, 97.22%) as a colorless oil which was directly used in the next step without further purification. MS (ESI, pos. ion) m/z 343.5 [M+H]⁺.

Step 4: To a solution of 2-methylpropan-2-yl 1,5-dimethyl-3-[(trimethylsilyl)oxy]-8-azabicyclo[3.2.1]oct-2-ene-8-carboxylate (1.6 g, 4.66 mmol) in acetonitrile (15 mL) was Selectfluor (1.65 g, 4.66 mmol) was added and the reaction mixture was stirred at room temperature overnight. After the reactants were consumed, the reaction was quenched with water, and diluted with EA (100 mL). The organic layer was collected, dried over sodium sulfate, and concentrated in vacuo. The crude mixture was purified by silica gel column chromatography with DCM/MeOH=20/1, to provided tert-butyl (1S,5R)-2,2-difluoro-1,5-dimethyl-3-oxo-8-azabicyclo[3.2.1]octane-8-carboxylate (837 mg, 3.04 mmol, 65.39%) as a white solid. MS (ESI, pos. ion) m/z 276.3 [M+H]⁺.

Step 5: 2-Methylpropan-2-yl (1S,2S,4R,5R)-2,4-difluoro-5-methyl-3-oxo-8-azabicyclo[3.2.1]octane-8-carboxylate (200 mg, 0.69 mmol) was dissolved in EtOH (1-5 mL) and NaBH₄ (32 mg, 0.83 mmol) was added at 80° C. The reaction was refluxed for 3 h. After the reactants were consumed, the mixture was cooled to room temperature, quenched with water and diluted with EA (50 mL). The mixture was washed with brine and the organic layer was collected, dried over sodium sulfate, and concentrated in vacuo. The crude mixture was purified by silica gel column chromatography with DCM/MeOH to afford tert-butyl (1S,3R,5R)-2,2-difluoro-3-hydroxy-1,5-dimethyl-8-azabicyclo[3.2.1]octane-8-carboxylate (80 mg, 0.29 mmol, 39.71%) as a white solid. MS (ESI, pos. ion) m/z 291.1 [M+H]⁺.

Step 6: To a solution of 2-methylpropan-2-yl (1S,2S,4R,5R)-2,4-difluoro-3-hydroxy-5-methyl-8-azabicyclo[3.2.1]octane-8-carboxylate (80 mg, 0.29 mmol) in THF (6 mL), NaH (16.5 mg, 0.41 mmol) was added and the reaction mixture was stirred for 20 min. Next 3,6-dibromo-1,2-diazine (85 mg, 0.35 mmol) was added and the reaction mixture was stirred at room temperature overnight. After the reactants were consumed, the mixture was cooled to room temperature, quenched with water and diluted with EA (50 mL). The mixture was washed with brine and the organic layer was collected, dried over sodium sulfate, and concentrated in vacuo. The crude mixture was purified by silica gel column chromatography with DCM/MeOH to afford tert-butyl (1S,3R,5R)-3-((6-bromopyridazin-3-yl)oxy)-2,2-difluoro-1,5-dimethyl-8-azabicyclo[3.2.1]octane-8-carboxylate (55 mg, 40.62%) as a white solid. MS (ESI, pos .ion) m/z 449.3 [M+H]⁺.

Step 7: To a solution of 6-(1,3-dimethylpyrazol-4-yl)-2,3-dihydro-1H-pyrrolo[4,3-c]pyridin-1-one (28 mg, 0.12) in dioxane (6 mL) was added 2-methylpropan-2-yl (1R,2R,3s,4S,5S)-3-[(6-bromo-1,2-diazin-3-yl)oxy]-2,4-difluoro-1,5-dimethyl-8-azabicyclo[3.2.1]octane-8-carboxylate (55 mg, 0.12 mmol), XantPhos Pd G4 (11.81 mg, 0.01 m-mol) and Cs₂CO₃ (119.91 mg, 0.37 mmol) and the reaction mixture was stirred at 90° C. overnight. After the reactants were consumed, the reaction was diluted with EA and H₂O. The organic layer was separated, washed with further saturated NaCl solution, and concentrated in vacuo and then purified using silica gel column chromatography eluting with DCM/MeOH to afford tert-butyl (1S,3R,5R)-3-((6-(6-(1,3-dimethyl-1H-pyrazol-4-yl)-1-oxo-1,3-dihydro-2H-pyrrolo[3,4-c]pyridin-2-yl)pyridazin-3-yl)oxy)-2,2-difluoro-1,5-dimethyl-8-azabicyclo[3.2.1]octane-8-carboxylate (15 mg, 0.03 mmol, 20.53%). MS (ESI, pos. ion) m/z 596.6 [M+H]⁺.

Step 8: 2-Methylpropan-2-yl 3-({6-[6-(1,3-dimethylpyrazol-4-yl)-1-oxo-2,3-dihydro-1H-pyrrolo[4,3-c]pyridin-2-yl]-1,2-diazin-3-yl}oxy)-2,4-difluoro-1,5-dimethyl-8-azabicyclo[3.2.1]octane-8-carboxylate (15 mg, 20.53%) was dissolved in HCl (5 mL, in dioxane). The reaction mixture was stirred at room temperature for 3 h. After the reactants were consumed, the reaction was concentrated in vacuo. The crude mixture was purified by silica gel column chromatography with DCM/MeOH to afford 2-(6-(((1S,3R,5R)-2,2-difluoro-1,5-dimethyl-8-azabicyclo[3.2.1]octan-3-yl)oxy)pyridazin-3-yl)-6-(1,3-dimethyl-1H-pyrazol-4-yl)-2,3-dihydro-1H-pyrrolo[3,4-c]pyridin-1-one (8.9 mg, 71.2%) as a white solid.

¹H NMR (DMSO-d₆) δ=8.99 (s, 1H), 8.76 (br d, J=9.6 Hz, 1H), 8.39 (s, 1H), 7.90 (s, 1H), 7.49 (br d, J=9.8 Hz, 1H), 5.72-5.84 (m, 1H), 5.25 (s, 2H), 3.82 (s, 3H), 2.48 (s, 3H), 2.27-2.40 (m, 2H), 1.81-2.03 (m, 2H), 1.60-1.66 (m, 2H), 1.22 (s, 6H). LC/MS (ESI): 496.5 [M+H]⁺.

### Example 2. Synthesis of 3-(1,3-dimethyl-1H-pyrazol-4-yl)-6-(6-(((1S,2R,3R,5R)-2-fluoro-1,5-dimethyl-8-azabicyclo[3.2.1]octan-3-yl)oxy)pyridazin-3-yl)-6,7-dihydro-5H-pyrrolo[3,4-b]pyridin-5-one (Compound No. 215)

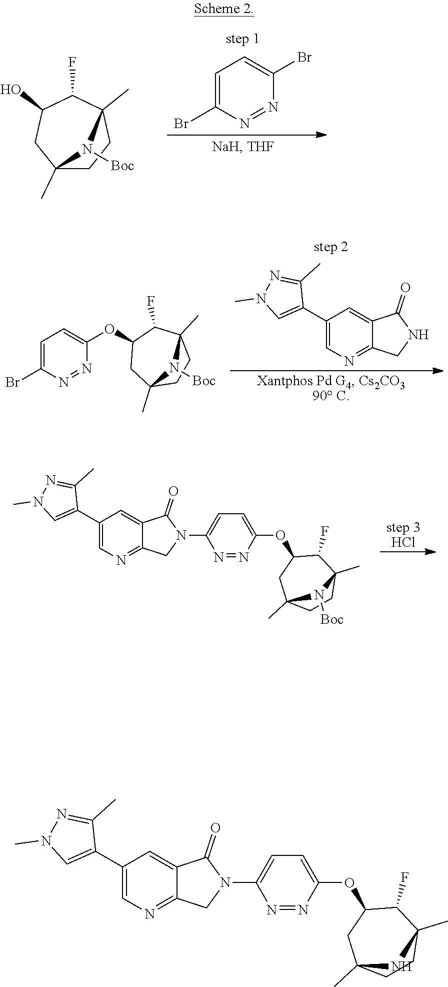

Step 1: To a solution of 2-methylpropan-2-yl (1S,3S,4S,5R)-4-fluoro-3-hydroxy-1,5-dimethyl-8-azabicyclo[3.2.1]octane-8-carboxylate (120 mg, 0.44 mmol) in THF (6 mL) was added NaH (15.80 mg, 0.66 mmol). The reaction mixture was stirred for 20 min and then 3,6-dibromo-1,2-diazine (156.64 mg, 0.66 mmol) was added. The reaction mixture was further stirred at room temperature overnight. After the reactants were consumed, the mixture was cooled to room temperature, quenched with water and diluted with EA (50 mL). The mixture was washed with brine and the organic layer was collected, dried over sodium sulfate, and concentrated in vacuo. The crude mixture was purified by silica gel column chromatography with DCM/MeOH to afford 2-methylpropan-2-yl (1S,3S,4S,5R)-3-[(6-bromo-1,2-diazin-3-yl)oxy]-4-fluoro-1,5-dimethyl-8-azabicyclo[3.2.1]octane-8-carboxylate (50 mg, 26.47%) as a white solid.

MS (ESI, pos. ion) m/z 431.3 [M+H]⁺. ¹H NMR (CHLOROFORM-d) δ=7.43 (d, 1H, J=9.2 Hz), 6.81 (d, 1H, J=9.2 Hz), 5.48-5.61 (m, 1H), 4.62 (dd, 1H, J=51.0, 8.4 Hz), 2.37-2.44 (m, 1H), 1.97-2.09 (m, 1H), 1.67-1.89 (m, 3H), 1.54-1.59 (m, 1H), 1.49 (s, 3H), 1.45 (s, 3H), 1.42 (s, 9H).

Step 2: To a solution of 3-(1,3-dimethylpyrazol-4-yl)-6,7-dihydro-5H-pyrrolo[4,3-b]pyridin-5-one (23 mg, 0.10 mmol) in dioxane (6 mL) were added 2-methylpropan-2-yl (1S,3S,4S,5R)-3-[(6-bromo-1,2-diazin-3-yl)oxy]-4-fluoro-1,5-dimethyl-8-azabicyclo[3.2.1]octane-8-carboxylate (43.36 mg, 0.10 mmol), XantPhos Pd G4 (9.70 mg, 0.01 mmol) and Cs₂CO₃ (98.33 mg, 0.30 mmol). The reaction mixture was stirred at 90° C. overnight. After the reactants were consumed, the reaction was diluted with EA and H₂O. The organic layer was separated, washed with saturated NaCl solution, and concentrated in vacuo and then purified using silica gel column chromatography eluting with DCM/MeOH to afford 2-methylpropan-2-yl (1R,2S,5S)-3-({6-[3-(1,3-dimethylpyrazol-4-yl)-5-oxo-6,7-dihydro-5H-pyrrolo[4,3-b]pyridin-6-yl]-1,2-diazin-3-yl}oxy)-2-fluoro-1,5-dimethyl-8-azabicyclo[3.2.1]octane-8-carboxylate (13 mg, 22.33%). MS (ESI, pos. ion) m/z 578.7 [M+H]⁺.

Step 3: 2-Methylpropan-2-yl (1R,2R,5S)-3-({6-[3-(1,3-dimethylpyrazol-4-yl)-5-oxo-6,7-dihydro-5H-pyrrolo[4,3-b]pyridin-6-yl]-1,2-diazin-3-yl}oxy)-2-fluoro-1,5-dimet-hyl-8-azabicyclo[3.2.1]octane-8-carboxylate (13 mg, 0.02 mmol) was dissolved in HCl (5 mL, in dioxane) and the reaction mixture was stirred at room temperature for 3 h. After the reactants were consumed, the mixture was concentrated in vacuo. The crude mixture was then purified by silica gel column chromatography with DCM/MeOH to afford 3-(1,3-dimethylpyrazol-4-yl)-6-(6-{[(1S,4S,5R)-4-fluoro-1,5-dimethyl-8-azabicyclo[3.2.1]octan-3-yl]oxy}-1,2-diazin-3-yl)-6,7-dihydro-5H-pyrrolo[4,3-b]pyridin-5-one (8 mg, 0.02 mmol, 74.44%) as a white solid.

¹H NMR (DMSO-d₆) δ=8.95-9.02 (m, 1H), 8.75 (d, J=9.6 Hz, 1H), 8.13-8.26 (m, 3H), 7.43 (d, J=9.6 Hz, 1H), 5.43-5.57 (m, 1H), 5.19 (s, 2H), 4.34-4.77 (m, 1H), 3.83 (s, 3H), 2.37 (s, 3H), 1.95-2.23 (m, 2H), 1.67-1.95 (m, 2H), 1.50-1.64 (m, 2H), 1.22 (s, 6H). LC/MS (ESI): 478.2 [M+H]⁺.

### Example 3. Synthesis of 2-(6-(2,7-diazaspiro[3.5]nonan-2-yl)pyridazin-3-yl)-6-(1,3-dimethyl-1H-pyrazol-4-yl)-5-fluoroisoindolin-1-one (Compound No. 154)

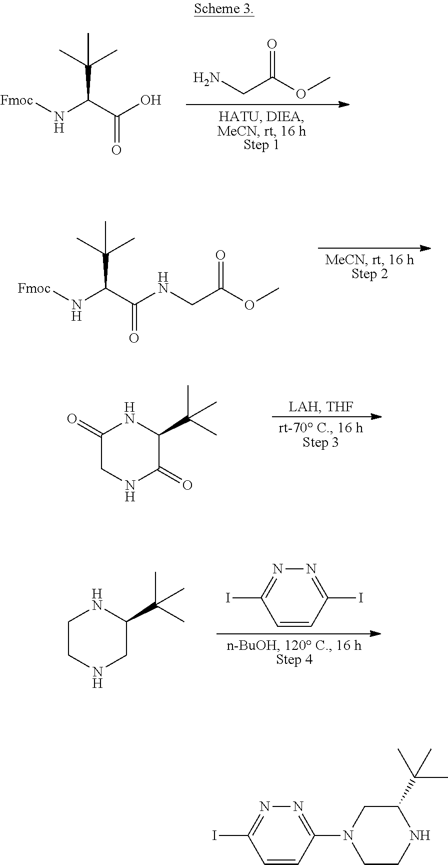

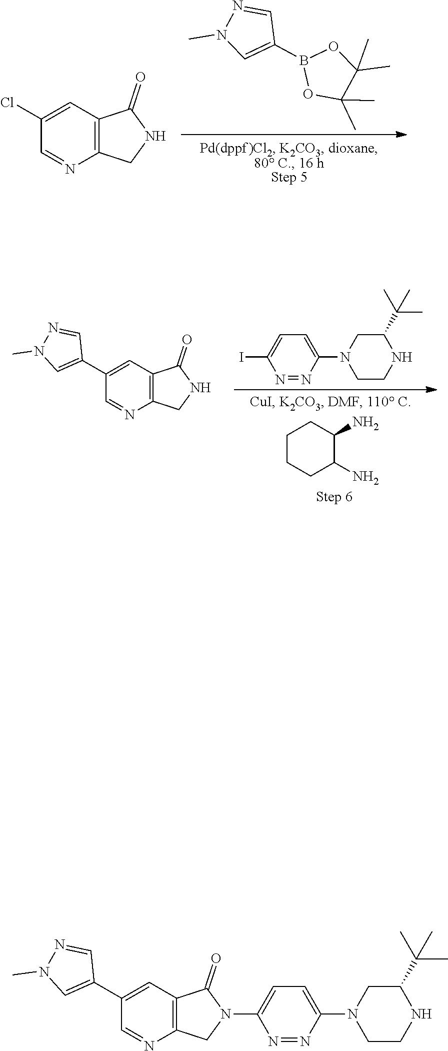

Step 1: To a solution of (2S)-2-({[(9H-fluoren-9-ylmethyl)oxy]carbonyl}amino)-3,3-dimethylbutanoic acid (10 g, 28.29 mmol, 1.0 eq) and methyl aminoacetate (3.0 g, 33.95 mmol, 1.2 eq) in acetonitrile (50 mL) was added DIEA (9.1 g, 70.74 mmol, 2.5 eq) and HATU (11.8 g, 31.12 mmol, 1.1 eq), and then the reaction mixture was stirred at room temperature for 16 h. After the reactants were consumed, the reaction mixture was filtered to afford 9H-fluoren-9-ylmethyl {[(7S)-8,8-dimethyl-3,6-dioxo-5-aza-2-oxanon-7-yl]amino}methanoate (9.5 g, 22.38 mmol, 79.09%) as sightly a yellow solid. MS m/z 425.5 [M+H]⁺.

Step 2: A solution of 9H-fluoren-9-ylmethyl {[(7S)-8,8-dimethyl-3,6-dioxo-5-aza-2-oxanon-7-yl]amino}methanoate (5.0 g, 11.78 mmol, 1.0 eq) in acetonitrile (100 mL) was stirred room temperature for 16 h. After the reactants were consumed, the solution was concentrated in vacuo to afford 9H-fluoren-9-ylmethyl {[(7S)-8,8-dimethyl-3,6-dioxo-5-aza-2-oxanon-7-yl]amino}methanoate (4.85 g, 11.43 mmol, 97.00%) as a yellow solid. MS m/z 171.2 [M+H]⁺.

Step 3: LiAlH₄ (2.14 g, 56.40 mmol, 6.0 eq) was added to a vigorous stirred solution of (3S)-3-(2-methylprop-2-yl)piperazine-2,5-dione (1.6 g, 9.40 mmol, 1.0 eq) in THF (20 mL) in an ice bath, and the mixture was further stirred at 70° C. for 16 h. After the reactants were consumed, the mixture was poured into KOH (6N) in an ice bath. The resulting mixture was further stirred for 1 h, filtered, and the filtrate was collected. The filtrate was extracted with EA, washed with brine, dried over Na₂SO₄, and concentrated in vacuo to provided (2S)-2-(2-methylprop-2-yl)piperazine (1.2 g, 8.44 mmol, 89.74%) as a yellow oil. MS m/z 143.2 [M+H]⁺.

Step 4: DIEA (1.13 g, 8.79 mmol, 1.46 mL, 2.5 eq) was added to a vigorous stirred solution of (2S)-2-(2-methylprop-2-yl)piperazine (0.5 g, 3.51 mmol, 1.0 eq) and 3,6-diiodo-1,2-diazine (1.17 g, 3.51 mmol, 1.0 eq) in n-BuOH (7 mL) at room temperature, and the mixture was stirred at 120° C. for 16 h. After the reactants were consumed, the mixture was concentrated in vacuo to give a residue, which was diluted with EA, washed with water and brine, dried over Na₂SO₄, and concentrated in vacuo to provided 6-iodo-3-[(3S)-3-(2-methylprop-2-yl)piperazin-1-yl]-1,2-diazine (450 mg, 1.30 mmol, 36.98%) as a yellow solid. MS m/z 347.2 [M+H]⁺.

Step 5: Pd(dppf)Cl₂ (49 mg, 0.06 mmol, 0.05 eq) was added to a vigorous stirred solution of 1-methyl-4-(4,4,5,5-tetramethyl-1,3,2-dioxaborolan-2-yl)pyrazole (250 mg, 1.20 mmol, 1.0 eq), 3-bromo-6,7-dihydro-5H-pyrrolo[4,3-b]pyridin-5-one (256 mg, 1.20 mmol, 1.0 eq) and K₂CO₃ (332 mg, 2.40 mmol, 2.0 eq) in dioxane (5 mL) and water (1 mL). The mixture was stirred at 80° C. for 16 h. Then the reaction mixture was filtered and the filtrate was concentrated in vacuo. The crude mixture was purified by silica gel column chromatography with DCM/MeOH 10/1, v/v to provide 3-(1-methylpyrazol-4-yl)-6,7-dihydro-5H-pyrrolo[4,3-b]pyridin-5-one (135 mg, 0.63 mmol, 52.51%) as a sightly yellow solid. MS m/z 215.2 [M+H]⁺.

Step 6: 6-(1-Methylpyrazol-4-yl)-2,3-dihydro-1H-pyrrolo[4,3-c]pyridin-1-one (50 mg, 0.23 mmol, 1.0 eq) and 6-iodo-3-[(3S)-3-(2-methylprop-2-yl)piperazin-1-yl]-1,2-diazine (80 mg, 0.23 mmol, 1.0 eq) was dissolved in DMF (2 mL) and CuI (4 mg, 0.02 mmol, 0.10 eq), K₂CO₃ (97 mg, 0.70 mmol, 3.0 eq) and (1R,2R)-cyclohexane-1,2-diamine (2.7 mg, 0.02 mmol, 0.10 eq) was added and the reaction mixture was stirred at 100° C. for 16 h. After the reactants were consumed, the mixture was filtered and the filtrate was concentrated to give a crude product which was purified by pre-HPLC to provided 2-{6-[(3S)-3-(2-methylprop-2-yl)piperazin-1-yl]-1,2-diazin-3-yl}-6-(1-methylpyrazol-4-yl)-2,3-dihydro-1H-pyrrolo[4,3-c]pyridin-1-one (24.19 mg, 21.55%) as a white solid.

¹H NMR (400 MHz, DMSO-d₆) δ=9.12 (d, J=2.0 Hz, 1H), 8.48 (d, J=9.9 Hz, 1H), 8.44 (s, 1H), 8.36 (d, J=2.1 Hz, 1H), 8.18 (s, 1H), 8.14 (s, 1H), 7.46 (d, J=10.0 Hz, 1H), 5.13 (s, 2H), 4.39-4.31 (m, 1H), 4.10 (br d, J=12.0 Hz, 1H), 3.89 (s, 3H), 3.08-3.03 (m, 1H), 2.86-2.55 (m, 3H), 2.35-2.31 (m, 1H), 0.96 (s, 9H). LC/MS (ESI): 433.5 [M+H]⁺.

### Example 4. Synthesis of 6-(6-((4-azadispiro[2.1.25.33]decan-9-yl)oxy)pyridazin-3-yl)-3-(1,3-dimethyl-1H-pyrazol-4-yl)-6,7-dihydro-5H-pyrrolo[3,4-b]pyridin-5-one (Compound No. 232)

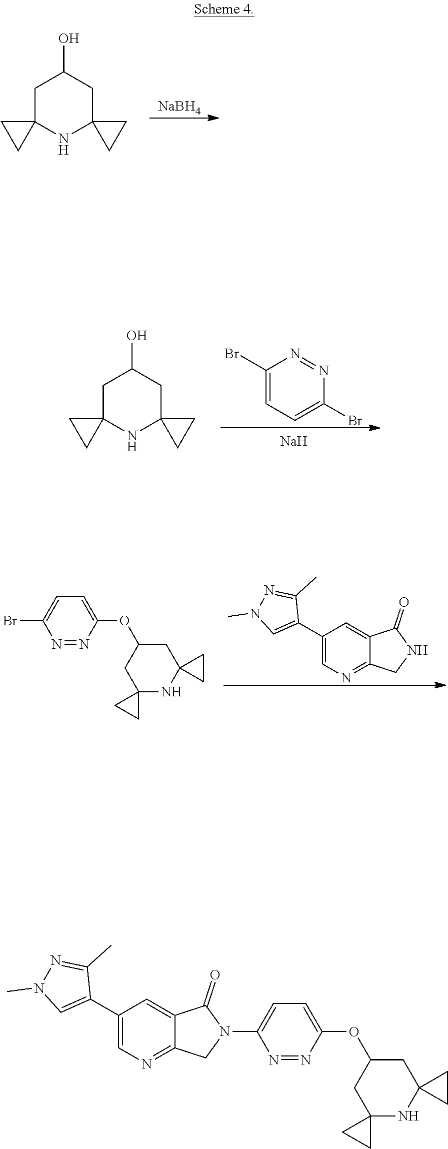

Step 1: To a 100 mL round-bottomed flask was added spiro[4-azaspiro[2.5]octane-5,1′-cyclopropane]-7-one (500 mg, 3.31 mmol) in MeOH (5 mL). This was followed with the addition of NaBH₄ (0.18 mL, 4.96 mmol) at 0° C. over 5 min to give an off-white suspension. The resulting mixture was stirred for 1 h at room temperature. LCMS showed the desired MS (M+H: 154.1). The resulting mixture was cooled to 0° C. and quenched with HCl (2 M, 0.5 mL) and then concentrated in vacuo. The resulting mixture was added to a silica column and was eluted with DCM:MeOH (20:1) (0.1% TEA). Spiro[4-azaspiro[2.5]octane-5,1′-cyclopropane]-7-ol (475 mg, 2.98 mmol, 90.00%) was obtained as a yellow solid. MS (ESI, pos. ion) m/z 154.1 [M+H]⁺.

Step 2: To a solution of spiro[4-azaspiro[2.5]octane-5,1′-cyclopropane]-7-ol (460 mg, 3.00 mmol) in THF (10 mL) was added NaH (270.20 mg, 4.50 mmol) at 0° C. and the resulting mixture was stirred for 30 min at room temperature. Next 3,6-dibromo-1,2-diazine (928.42 mg, 3.90 mmol) was added at 0° C. and the resulting mixture was stirred for 12 h at 60° C. The reaction mixture was quenched with saturated NH₄C₁ solution (50 mL) and the aqueous layer was extracted with EA (50 mL*3). The organic layer was concentrated under vacuum and residue was purified by column chromatography (SiO₂, PE:EA=0~50%, 30 min) to afford 7-[(6-bromo-1,2-diazin-3-yl)oxy]spiro[4-azaspiro[2.5]octane-5,1′-cyclopropane](360 mg, 1.11 mmol, 37.11%). MS (ESI, pos. ion) m/z 310.05 [M+H]⁺.

Step 3: To a flask containing 3-(1,3-dimethylpyrazol-4-yl)-6,7-dihydro-5H-pyrrolo[4,3-b]pyridin-5-one (40.47 mg, 0.18 mmol) in dioxane (2 mL) was added 7-[(6-bromo-1,2-diazin-3-yl)oxy]spiro[4-azaspiro[2.5]octane-5,1′-cyclopropane](50 mg, 0.16 mmol), Cs₂CO₃ (157.56 mg, 0.48 mmol) and 3-(1,3-dimethyl-1H-pyrazol-4-yl)-6,7-dihydro-5H-pyrrolo[3,4-b]pyridin-5-one (26.11 mg, 0.02 mmol) under nitrogen. The mixture was stirred at 90° C. for 18 h. The reaction mixture was concentrated under vacuum. The residue was dissolved in DCM (3 mL) and ACN (2 mL) was added drop wise to get white slurry. The off-white solid was filtered to obtain 3-(1,3-dimethylpyrazol-4-yl)-6-[6-(spiro[4-azaspiro[2.5]octane-5,1′-cyclopropane]-7-yloxy)-1,2-diazin-3-yl]-6,7-dihydro-5H-pyrrolo[4,3-b]pyridin-5-one (4 mg, 0.01 mmol, 5.05%) as an off-white solid.

MS (ESI, pos. ion) m/z 458.3 [M+H]⁺. ¹H NMR (400 MHz, CDCl₃) δ=8.89-8.79 (m, 2H), 8.14 (d, J=1.9 Hz, 1H), 7.56 (s, 1H), 7.05 (d, J=9.6 Hz, 1H), 5.75-5.65 (m, 1H), 5.28 (s, 2H), 3.92 (s, 3H), 2.44 (s, 3H), 2.02-1.89 (m, 2H), 1.86-1.77 (m, 2H), 0.75-0.52 (m, 6H), 0.46-0.39 (m, 2H).

### Example 5. Synthesis of 3-(1,3-dimethyl-1H-pyrazol-4-yl)-6-(3-(methyl(2,2,6,6-tetramethylpiperidin-4-yl)amino)-1,2,4-triazin-6-yl)-6,7-dihydro-5H-pyrrolo[3,4-b]pyridin-5-one (Compound No. 162)

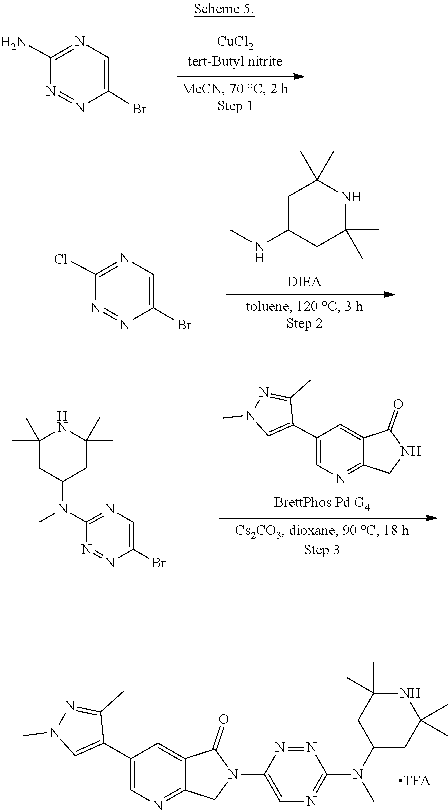

Step 1: To a solution of 6-bromo-1,2,4-triazin-3-amine (2.0 g, 11.43 mmol) and CuCl₂ (1.47 g, 14.86 mmol) in MeCN (25 mL) was added isoamyl nitrite (2.18 mL, 18.29 mmol) and the reaction mixture was stirred at 70° C. for 2 h. Nex the reaction mixture was concentrated and the residue was purified by column chromatography (SiO₂, PE:EA=10:1) to give 6-bromo-3-chloro-1,2,4-triazine (220 mg, 1.13 mmol, 9.90%) as a yellow solid.

Step 2: To a solution of 6-bromo-3-chloro-1,2,4-triazine (200 mg, 1.03 mmol) and 2,2,6,6-tetramethyl-4-(methylamino)hexahydropyridine (192.71 mg, 1.13 mmol) in toluene (10 mL) was added DIEA (0.34 mL, 2.06 mmol) and the reaction mixture was stirred at 120° C. for 3 h. LCMS showed the reaction was completed. Next the reaction mixture was concentrated and the residue was purified by column chromatography (SiO₂, DCM:MeOH=10:1) to give 6-bromo-3-[methyl(2,2,6,6-tetramethylhexahydropyridin-4-yl)amino]-1,2,4-triazine (0.23 g, 67.65%) as a yellow solid.

Step 3: To a solution of 3-(1,3-dimethylpyrazol-4-yl)-6,7-dihydro-5H-pyrrolo[4,3-b]pyridin-5-one (35 mg, 0.15 mmol) and 6-bromo-3-[methyl(2,2,6,6-tetramethylhexahydropyridin-4-yl)amino]-1,2,4-triazine (52 mg, 0.16 mmol) in dioxane (10 mL) was added BrettPhos Pd G₄ (13.61 mg, 0.01 mmol) and Cs₂CO₃ (146.62 mg, 0.45 mmol). The reaction mixture was stirred at 90° C. for 16 h under N₂. LCMS showed the reaction was completed. Next the reaction mixture was filtered and concentrated. The residue was purified by prep-HPLC (TFA) to give 3-(1,3-dimethylpyrazol-4-yl)-6-{3-[methyl(2,2,6,6-tetramethylhexahydropyridin-4-yl)amino]-1,2,4-triazin-6-yl}-6,7-dihydro-5H-pyrrolo[4,3-b]pyridin-5-one (8 mg, 10.97%) as a yellow solid.

MS (ESI, pos. ion) m/z: 476.3[M+H]⁺. ¹H NMR (400 MHz, DMSO-d₆) δ=9.41 (s, 1H), 8.95 (d, J=2.0 Hz, 1H), 8.22-8.14 (m, 2H), 5.14 (m, 3H), 3.83 (s, 3H), 3.09 (s, 3H), 2.35 (s, 3H), 1.99-1.82 (m, 4H), 1.51 (s, 6H), 1.43 (s, 6H), 1H not observed (NH).

### Example 6. Synthesis of 6-(1,3-dimethyl-1H-pyrazol-4-yl)-2-(6-(((1R,3s,5S)-1,5-dimethyl-8-azabicyclo[3.2.1]octan-3-yl)(methyl)amino)pyridazin-3-yl)-4-fluoroisoindolin-1-one (Compound No. 274)

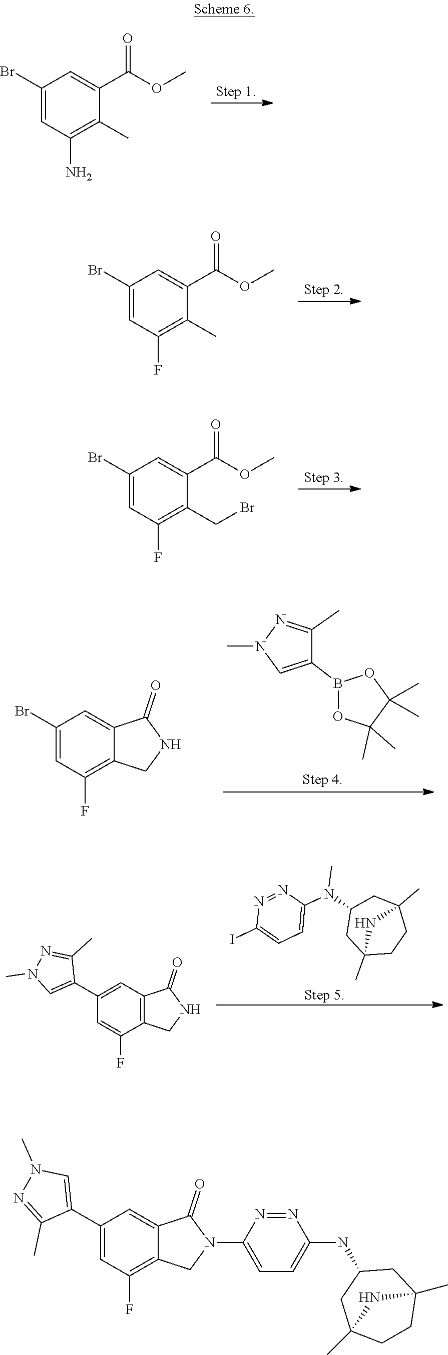

Step 1: To a solution of methyl 3-amino-5-bromo-2-methylbenzoate (1.0 g, 4.10 mmol) in DCM (10 mL) was added nitrosonium tetrafluoroborate (480 mg, 4.10 mmol) portion wise at 0° C. under N₂ to obtain a yellow solution which was stirred at 0° C. for 1 h. Then oxylene (30 mL) was added the reaction mixture was refluxed at 130° C. for 3 h. Next the reaction mixture was cooled to room temperature and poured into H₂O. The aqueous layer was extracted EA. The combined organic layers were washed with brine, dried over MgSO₄, filtered, and concentrated. The crude mixture was purified by silica gel column chromatography with PE/EA 10/1, v/v to obtain methyl 5-bromo-3-fluoro-2-methylbenzoate (860 mg, 85%).

Step 2: To the solution of 5-bromo-3-fluoro-2-methylbenzoate (860 mg, 3.5 mmol) and NBS (623 mg, 3.5 mmol) in (trifluoromethyl)benzene (10 mL), was added AIBN (57 mg, 0.35 mmol). The reaction mixture was stirred under N₂ at 100° C. for 16 h. Next, the reaction mixture was cooled to room temperature and poured into H₂O. The aqueous layer was extracted EA. The combined organic layers were washed with brine, dried over MgSO₄, filtered, and concentrated. The crude mixture was purified by silica gel column chromatography with PE/EA 10/1, v/v to obtain methyl 5-bromo-2-(bromomethyl)-3-fluorobenzoate (800 mg, 70%).

Step 3: Methyl 5-bromo-2-(bromomethyl)-3-fluorobenzoate (800 mg, 2.5 mmol) was dissolved in THF (7 mL) and NH₄OH (7 mL) was added. The reaction was stirred at room temperature for 4 h and was then filtered and concentrated under vacuum. The crude product was used directly in the next step without further purification.

Step 4: To the solution of 6-bromo-4-fluoroisoindolin-1-one (100 mg, 0.43 mmol) and 1,3-dimethyl-4-(4,4,5,5-tetramethyl-1,3,2-dioxaborolan-2-yl)-1H-pyrazole (96 mg, 0.43 mmol) in dioxane:H₂O (5:1) (2 mL: 0.4 mL), potassium carbonate (118 mg, 0.86 mmol) and PdCl₂(dppf) (31 mg, 0.043 mmol) were added. The reaction mixture was stirred under N₂ at 100° C. for 16 h. Then the mixture was cooled, filtered through a pad of Celite, and the filter cake was washed with EA. The filtrate was washed with brine and the organic layer was collected, dried over sodium sulfate, and concentrated in vacuo. The crude mixture was purified by silica gel column chromatography with DCM/MeOH 20/1, v/v to obtain 6-(1,3-dimethyl-1H-pyrazol-4-yl)-4-fluoroisoindolin-1-one (85 mg, 81%) as a yellow solid.

MS m/z 231.0 [M+H]. ¹H NMR (400 MHz, DMSO-d6) δ=8.51-8.33 (m, 2H), 8.04 (s, 1H), 7.64-7.60 (m, 1H), 7.55 (d, J=10.3 Hz, 1H), 7.31 (d, J=10.0 Hz, 1H), 5.13 (s, 2H), 5.11-5.03 (m, 1H), 3.81 (s, 3H), 2.90 (s, 3H), 2.36 (s, 3H), 2.16 (br d, J=8.1 Hz, 2H), 2.03-1.87 (m, 4H), 1.80 (br dd, J=5.4, 13.6 Hz, 2H), 1.45 (s, 6H).

### Example 7. Synthesis of 6-(6-(((1R,3s,5S)-1,5-dimethyl-8-azabicyclo[3.2.1]octan-3-yl)oxy)pyridazin-3-yl)-2-(2,4-dimethylthiazol-5-yl)-5,6-dihydro-7H-pyrrolo[3,4-d]pyrimidin-7-one (Compound No. 111)

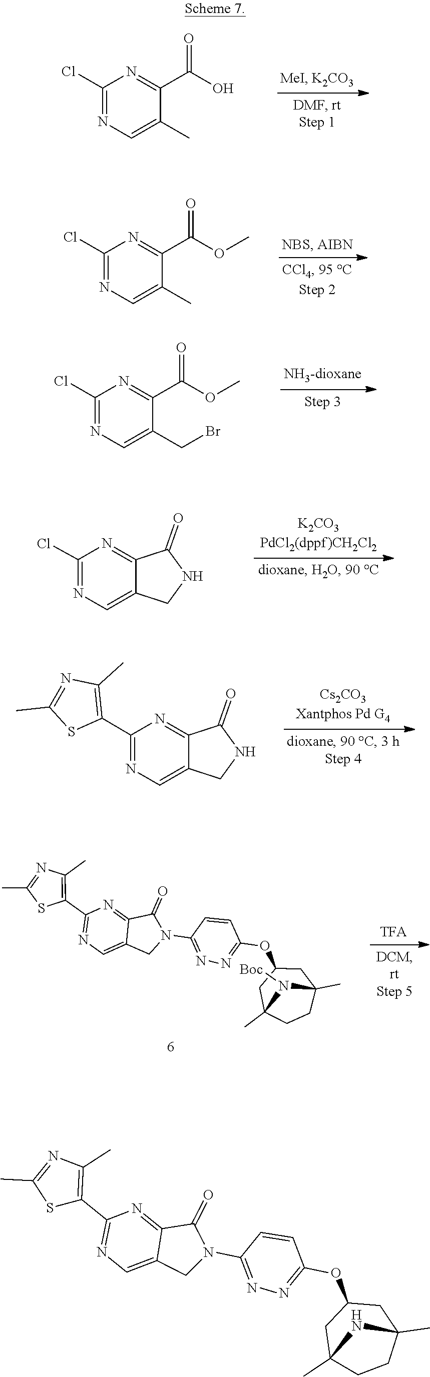

Step 1: To a mixture of 2-chloro-5-methylpyrimidine-4-carboxylic acid (5.0 g, 28.97 mmol) and K₂CO₃ (8.01 g, 57.95 mmol) in DMF (10 mL) was added Mel (5.41 mL, 86.92 mmol) at room temperature, and the mixture was stirred for 16 h. After the reaction was completed, the mixture was filtrated, quenched with H₂O, and extracted with EA. The combined organic layers were washed with aq. NaCl, dried over Na₂SO₄, and concentrated. The resulting residue was purified with chromatography on silica gel PE:EA=5:1 to give 2-chloro-5-methylpyrimidine-4-carboxylate (5.0 g, 92.49%) as a white solid.

Step 2: To a mixture of methyl 2-chloro-5-methylpyrimidine-4-carboxylate (5.0 g, 26.80 mmol) in CCl₄ (100 mL) was added NBS (6.20 g, 34.84 mmol) and AIBN (0.44 g, 2.68 mmol) at room temperature under N₂, and the resulting mixture was stirred at 95° C. for 2 h. Next NBS (2.38 g, 13.40 mmol) was added and the mixture was stirred at 95° C. for 1 h. Then NBS (1.43 g, 8.04 mmol) was added and the mixture was stirred at 95° C. for 1 h. After the reaction was completed, the mixture was cooled to room temperature and was quenched with H₂O and extracted with DCM. The combined organic layers were washed with aq. NaCl, dried over Na₂SO₄, and concentrated. The residue was purification by chromatography on silica gel PE:EA=5:1 to give 5-(bromomethyl)-2-chloropyrimidine-4-carboxylate (4.0 g, 56.26%) as an oil.

Step 3: A mixture of methyl 5-(bromomethyl)-2-chloropyrimidine-4-carboxylate (4.0 g, 15.07 mmol) in NH₃-dioxane (30 mL) was stirred at room temperature for 1 h. LCMS analysis showed the reaction was completed. The mixture was the filtrated and concentrated to afford 2-chloro-6,7-dihydro-5H-pyrrolo[4,3-d]pyrimidin-7-one (1.3 g, 50.98%) as a red solid.

Step 4: To a mixture of 2-chloro-6,7-dihydro-5H-pyrrolo[4,3-d]pyrimidin-7-one (110 mg, 0.65 mmol) and 2,4-dimethyl-5-(4,4,5,5-tetramethyl-1,3,2-dioxaborolan-2-yl)-1,3-thiazole (201.67 mg, 0.84 mmol) in dioxane: H₂O=5:1 (6 mL) was added PdCl₂(dppf) CH₂Cl₂ (52.98 mg, 0.06 mmol) and K₂CO₃ (268.95 mg, 1.95 mmol) at room temperature under N₂, and the mixture was stirred at 95° C. for 3 h. LCMS analysis showed the reaction was completed. Next the mixture was concentrated and the residue was purification by chromatography on silica gel DCM:MeOH=10:1 to give 2-(2,4-dimethyl-1,3-thiazol-5-yl)-6,7-dihydro-5H-pyrrolo[4,3-d]pyrimidin-7-one (0.1 g, 62.50%) as a yellow solid.

Step 5: To a mixture of 2-(2,4-dimethyl-1,3-thiazol-5-yl)-6,7-dihydro-5H-pyrrolo[4,3-d]pyrimidin-7-one (65 mg, 0.26 mmol), 2-methylpropan-2-yl (1R,3s,5S)-3-[(6-bromo-1,2-diazin-3-yl)oxy]-1,5-dimethyl-8-azabicyclo[3.2.1]octane-8-carboxylate (119.70 mg, 0.29 mmol) in dioxane (20 mL) was added XantPhos Pd G4 (42.76 mg, 0.03 mmol) and Cs₂CO₃ (257.97 mg, 0.79 mmol) at room temperature under N₂, and the mixture was stirred at 95° C. for 16 h. LCMS analysis showed the reaction was completed. Next the mixture was filtrated and concentrated to afford 2-methylpropan-2-yl (1S,3s,5R)-3-({6-[2-(2,4-dimethyl-1,3-thiazol-5-yl)-7-oxo-6,7-dihydro-5H-pyrrolo[4,3-d]pyrimidin-6-yl]-1,2-diazin-3-yl}oxy)-1,5-dimethyl-8-azabicyclo[3.2.1]octane-8-carboxylate (150 mg, 98.39%) as a yellow solid which was used in the next step without further purification.

Step 6: To a mixture of 2-methylpropan-2-yl (1S,3s,5R)-3-({6-[2-(2,4-dimethyl-1,3-thiazol-5-yl)-7-oxo-6,7-dihydro-5H-pyrrolo[4,3-d]pyrimidin-6-yl]-1,2-diazin-3-yl}oxy)-1,5-dimethyl-8-azabicyclo[3.2.1]octane-8-carboxylate (100 mg, 0.17 mmol) in DCM (10 mL) was added TFA (1 mL) at room temperature and the mixture was stirred at room temperature for 0.5 h. LCMS analysis showed the reaction was completed. Next the mixture was concentrated and the residue was purified with pre-HPLC(TFA) to give 2-(2,4-dimethyl-1,3-thiazol-5-yl)-6-(6-{[(1S,3s,5R)-1,5-dimethyl-8-azabicyclo[3.2.1]octan-3-yl]oxy}-1,2-diazin-3-yl)-6,7-dihydro-5H-pyrrolo[4,3-d]pyrimidin-7-one (72 mg, 87.09%) as a white solid.

MS (ESI, pos. ion) m/z:478.3[M+H]⁺. ¹HNMR (DMSO-d₆) δ=9.32 (s, 1H), 8.94 (br s, 1H), 8.86 (br s, 1H), 8.74 (dd, J=9.6, 0.9 Hz, 1H), 7.43 (d, J=9.5 Hz, 1H), 5.49-5.60 (m, 1H), 5.21 (s, 2H), 2.84 (s, 3H), 2.67 (s, 3H), 2.42-2.48 (m, 2H), 2.17 (br d, J=8.1 Hz, 2H), 1.89 (br d, J=8.3 Hz, 2H), 1.76-1.85 (m, 2H), 1.46 (s, 6H).

### Example 8. Synthesis of (2-(6-(((1R,3s,5S)-1,5-dimethyl-8-azabicyclo[3.2.1]octan-3-yl)oxy)pyridazin-3-yl)-6-(2,4-dimethylthiazol-5-yl)-2,3-dihydro-1H-pyrrolo[3,4-c]pyridin-1-one (Compound No. 69)

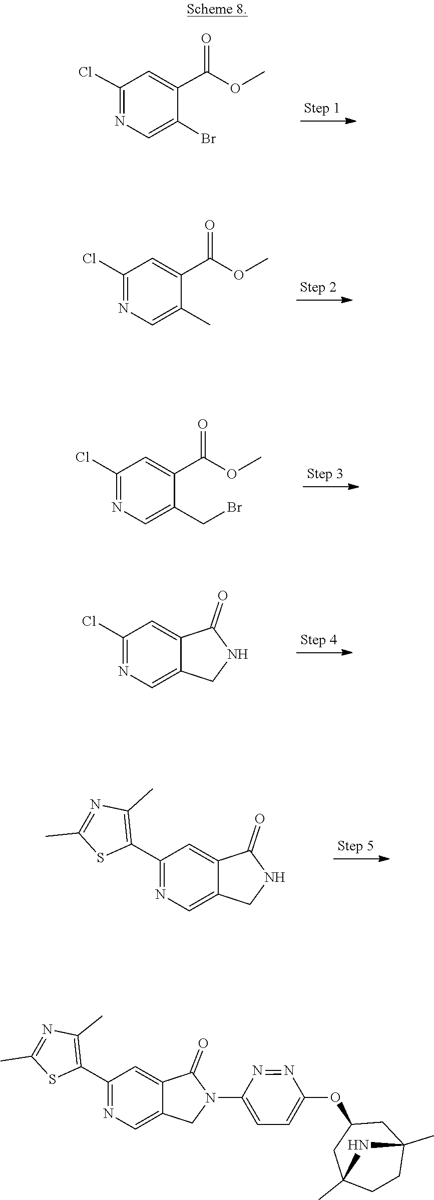

Step 1: To a solution of methyl 5-bromo-2-chloroisonicotinate (5 g, 20 mmol, 1.0 eq), methylboronic acid (1.79 g, 29.8 mmol, 1.5 eq), K₃PO₄ (14.8 g, 69.9 mmol, 3.5 eq), P(Cy)₃ (1.11 g, 3.97 mmol, 0.2 eq) and Pd(OAc)₂ (448 mg, 2.00 mmol, 0.1 eq) in toluene (95 mL) and H₂O (4.5 mL) was degassed and purged with N₂ three times. Then the mixture was stirred at 100° C. for 2 hours under N₂ atmosphere. When full conversion was achieved, the reaction mixture was quenched with water (40 mL) and extracted with ethyl acetate (3×20 mL). The extracts were dried over anhydrous sodium sulfate, filtered and concentrated in vacuo to get the crude residue. The residue was purified by column chromatography (SiO₂, Petroleum ether/Ethyl acetate=20/1 to 15/1) to give methyl 2-chloro-5-methylisonicotinate (2 g, 54% yield) as a yellow solid.

Step 2: 2-Chloro-5-methylisonicotinate (15.0 g, 81.7 mmol, 1.0 eq), N-bromosuccinimide (20.3 g, 113.9 mmol, 1.4 eq) and benzoyl peroxide (448.0 mg, 1.85 mmol, 0.03 eq) were combined with carbon tetrachloride (250 mL). The reaction mixture was heated to 80° C. and stirred for 3 h. The reaction mixture was cooled to rt and concentrated in vacuum. The obtained residue was purified by silica gel column chromatography with PE/EA to obtain methyl 5-(bromomethyl)-2-chloroisonicotinate (17.0 g, 79%) as a brown solid. LC/MS (ESI): 263.9, 265.9 [M+H]⁺.

Step 3: Methyl 5-(bromomethyl)-2-chloroisonicotinate (17.0 g, 64.6 mmol, 1.0 eq) was suspended in 28% aqueous ammonium hydroxide and the mixture was kept stirring at rt for 8 hrs. Then the mixture was filtered, and the residue was washed with water and methanol. The solid was collected, dried under vacuum and used in the next step without further purification. LC/MS (ESI): 169.0, 171.0 [M+H]⁺.

Step 4: 6-Chloro-2,3-dihydro-1H-pyrrolo[3,4-c]pyridin-1-one (336.0 mg, 2.0 mmol, 1.0 eq), 2,4-dimethyl-5-(4,4,5,5-tetramethyl-1,3,2-dioxaborolan-2-yl)thiazole) (526.1 mg, 2.20 mmol, 1.1 eq), aqueous potassium carbonate (2 M, 8.0 mL) and PdCl₂(dppf). CH₂Cl₂ adduct (146.3 mg, 0.2 mmol, 0.1 eq) were charged to a three-neck 100 mL round bottom flask. The flask was evacuated and backfilled three times with nitrogen. Anhydrous dioxane (9 mL) was added and the mixture was heated to 85° C. overnight. Then the mixture was cooled, filtered through a pad of Celite, washing the filter cake with EtOAc (100 mL). The filtrate was washed with washed with brine, then the organic layer was collected and dried over sodium sulfate, then concentrated in vacuo. The crude mixture was purified by silica gel column chromatography with DCM/MeOH 20/1, v/v to obtain 6-(2,4-dimethylthiazol-5-yl)-2,3-dihydro-1H-pyrrolo[3,4-c]pyridin-1-one (245 mg, 52%) as brown solid. LC/MS (ESI): 246.0 [M+H]⁺.

Step 5: To a round bottom flask charged with 6-(2,4-dimethylthiazol-5-yl)-2,3-dihydro-1H-pyrrolo[3,4-c]pyridin-1-one (36.7 mg, 0.15 mmol, 1.0 eq), tert-butyl (1R,3s,5S)-3-((6-bromopyridazin-3-yl)oxy)-1,5-dimethyl-8-azabicyclo[3.2.1]octane-8-carboxylate (61.8 mg, 0.15 mmol, 1.1 eq), Cs₂CO₃ (148.9 mg, 0.46 mmol, 3.0 eq), and XantPhos Pd G4 (14.2 mg, 0.015 mmol, 0.1 eq) was added 1,4-dioxane (4 mL). The reaction mixture was degassed with argon for 5 min then heated at 90° C. for 8 hrs. The reaction mixture was cooled to RT and diluted with EtOAc. The organic phase was washed with brine, dried over sodium sulfate, and concentrated under vacuum. The crude mixture was purified by silica gel column chromatography with DCM/MeOH 20/1, v/v to obtain tert-butyl (1R,3s,5S)-3-((6-(6-(2,4-dimethylthiazol-5-yl)-1-oxo-1,3-dihydro-2H-pyrrolo[3,4-c]pyridin-2-yl)pyridazin-3-yl)oxy)-1,5-dimethyl-8-azabicyclo[3.2.1]octane-8-carboxylate (42.2 mg), which was dissolved HCl in dioxane (2 mL, 4.0 mol/L). The reaction was stirred at RT for 1 h, and then concentrated. The residue obtained was purified by RP HPLC using MeCN and H₂O as eluent to provide 2-(6-(((1R,3s,5S)-1,5-dimethyl-8-azabicyclo[3.2.1]octan-3-yl)oxy)pyridazin-3-yl)-6-(2,4-dimethylthiazol-5-yl)-2,3-dihydro-1H-pyrrolo[3,4-c]pyridin-1-one (12.2 mg, 17.3%) as a white solid.

¹H NMR (DMSO-d6, 400 MHz) δ=9.06 (s, 1H), 8.69 (d, J=9.6 Hz, 1H), 8.24 (s, 1H), 7.92 (s, 1H), 7.35 (d, J=9.6 Hz, 1H), 5.49 (s, 1H), 5.28 (s, 2H), 2.65 (s, 6H), 2.22 (br dd, J=5.9, 12.3 Hz, 2H), 1.88 (br d, J=7.4 Hz, 2H), 1.60 (br d, J=6.9 Hz, 2H), 1.49 (br t, J=11.4 Hz, 2H), 1.25 (s, 6H); LC/MS (ESI): 477.2 [M+H]⁺.

### Example 9. Synthesis of 6-(1,3-dimethyl-1H-pyrazol-4-yl)-2-(6-(((1R,3s,5S)-1,5-dimethyl-8-azabicyclo[3.2.1]octan-3-yl)oxy)pyridazin-3-yl)-2,3-dihydro-1H-pyrrolo[3,4-c]pyridin-1-one (Compound No. 72)

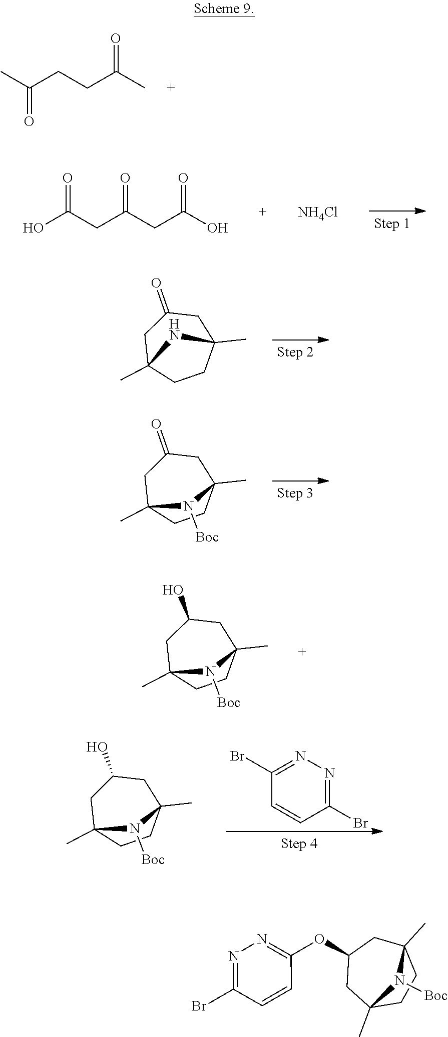

Step 1: Potassium hydroxide (74.8 g, 1.33 mol, 4.5 eq) in water (60 mL) was added to a vigorous stirred solution of 3-oxopentanedioic acid (82.8 g, 0.566 mol, 4.5 eq) and hexane-2,5-dione (34.0 g, 35.0 mL, 0.298 mol, 1.0 eq) in water (120 mL) at 0° C. After the addition, ammonium chloride (47.8 g, 0.896 mol, 3.0 eq) and sodium acetate (49.0 g, 0.596 mol, 2.0 eq) in water (200 mL) were added keeping the temperature at 5° C. The pH value was adjusted to ~9 by using solid KOH. The mixture was stirred at room temperature for 4 days during which time the pH value was adjusted to ~9 by using additional solid KOH. The solution was diluted with 100 mL of water, cooled to 5° C. with vigorous stirring and acidified with pure sulfuric acid until pH=2 (−60 mL). Gas evolution was observed during the acidification. Water layer was divided in two batches. Each batch was washed with EtOAc (2×100 mL) and basified with solid KOH with cooling until pH=10. The water layer was extracted with 15% MeOH in DCM (4×100 mL). The combined organic layers were washed with brine (100 mL), dried over sodium sulfate, filtered, and concentrated in vacuo to afford 1,5-dimethyl-8-azabicyclo[3.2.1]octan-3-one (17.3 g, 37.5%) as a dark brown oil. LC/MS (ESI): 154.1 [M+H]⁺.

Step 2: To an ice-cold solution of 1,5-dimethyl-8-azabicyclo[3.2.1]octan-3-one (3 g, 19.6 mmol, 1.0 eq) of DCM (60 mL) was added pyridine (4.0 mL, 49.0 mmol, 2.5 eq). Triphosgene (2.33 g, 7.84 mmol, 0.4 eq) was dissolved in DCM (20 mL) and added to the above solution dropwise. After 30 minutes, t-BuOH (3.78 mL, 39.2 mmol, 2.0 eq) was added and the mixture was gradually warmed to RT and stirred for 8 hrs. The reaction was quenched with water and diluted with DCM (300 mL). The mixture was washed with 1N HCl (1×50 mL), saturated sodium bicarbonate (1×50 mL), and brine (1×50 mL). The organic layer was collected and dried over sodium sulfate, and then concentrated in vacuo. The crude mixture was purified by silica gel column chromatography with PE/EA 20/1, v/v to provide tert-butyl (1R,5S)-1,5-dimethyl-3-oxo-8-azabicyclo[3.2.1]octane-8-carboxylate (2.3 g, 9.1 mmol, 46.3%) as a sightly yellow oil. LC/MS (ESI): 254.2 [M+H]⁺.

Step 3: (1R,5S)-1,5-Dimethyl-3-oxo-8-azabicyclo[3.2.1]octane-8-carboxylate (2.3 g, 9.1 mmol) was dissolved in EtOH (50 mL) and the solution was heated to reflux. Sodium borohydride (336 mg, 1.0 eq) and was added the mixture was stirred for 15 min. The mixture was cooled to RT and quenched with aqueous ammonium chloride. The organic solvent was removed under vacuum and the residue was redissolved in EtOAc (100 mL). The mixture was washed with brine, and the organic layer was collected and dried over sodium sulfate, and then concentrated in vacuo. The crude mixture was purified by silica gel column chromatography with PE/EtOAc to provided tert-butyl (1R,3r,5S)-3-hydroxy-1,5-dimethyl-8-azabicyclo[3.2.1]octane-8-carboxylate (1.2 g, 52%, PE/EtOAc 3/1, v/v) and tert-butyl (1R,3s,5S)-3-hydroxy-1,5-dimethyl-8-azabicyclo[3.2.1]octane-8-carboxylate (1.1 g, 48%, PE/EtOAc 2/1, v/v) as white solid. LC/MS (ESI): 256.2 [M+H]⁺.

Step 4: To a solution of (1R,3s,5S)-3-hydroxy-1,5-dimethyl-8-azabicyclo[3.2.1]octane-8-carboxylate (1.10 g g, 4.34 mmol, 1.0 eq) in anhydrous THF (20 mL) was added NaH (60 wt %, 208 mg, 5.21 mmol, 1.2 eq). The solution was stirred at room temperature for 30 min, then 3,6-dibromopyridazine (1.24 g, 5.21 mmol, 1.2 eq) was added and the mixture was stirred at RT for 8 hrs. The reaction mixture was quenched with saturated ammonium chloride and diluted with EtOAc (100 mL). The mixture was washed with brine, dried over sodium sulfate, and then concentrated in vacuo. The crude mixture was purified by silica gel column chromatography with PE/EA 20/1, v/v to provide tert-butyl (1R,3s,5S)-3-((6-bromopyridazin-3-yl)oxy)-1,5-dimethyl-8-azabicyclo[3.2.1]octane-8-carboxylate (1.28 g, 72%) as white solid. LC/MS (ESI): 412.2, 414.2 [M+H]⁺.

Step 5: A mixture of 6-(1,3-dimethylpyrazol-4-yl)-2,3-dihydro-1H-pyrrolo[4,3-c]pyridin-1-one (210 mg, 0.92 mmol), 2-methylpropan-2-yl (1R,3s,5S)-3-[(6-bromo-1,2-diazin-3-yl)oxy]-1,5-dimethyl-8-azabicyclo[3.2.1]octane-8-carboxylate (379.36 mg, 0.92 mmol) in dioxane (30 mL) was added Xantphos Pd G4 (149.05 mg, 0.09 mmol), Cs₂CO₃ (897.04 mg, 2.76 mmol) at RT under N₂ and the mixture was stirred at 95° C. for 16 h. The reaction mixture was concentrated and the residue was purification by chromatography on silica gel and eluted with DCM:MeOH=10:1 to give 2-methylpropan-2-yl (1R,3s,5S)-3-({6-[6-(1,3-dimethylpyrazol-4-yl)-1-oxo-2,3-dihydro-1H-pyrrolo[4,3-c]pyridin-2-yl]-1,2-diazin-3-yl}oxy)-1,5-dimethyl-8-azabicyclo[3.2.1]octane-8-carboxylate (400 mg, 77.68%) as a yellow solid.

Step 6: A mixture of 2-methylpropan-2-yl (1R,3s,5S)-3-({6-[6-(1,3-dimethylpyrazol-4-yl)-1-oxo-2,3-dihydro-1H-pyrrolo[4,3-c]pyridin-2-yl]-1,2-diazin-3-yl}oxy)-1,5-dimethyl-8-azabicyclo[3.2.1]octane-8-carboxylate (360 mg, 0.64 mmol) in DCM (10 mL) was added HCl-dioxane (20 mL) at RT and the mixture was stirred or 1 h. The reaction mixture was concentrated and the residue was purification by pre-HPLC(HCl) to give 6-(1,3-dimethylpyrazol-4-yl)-2-(6-{[(1R,3s,5S)-1,5-dimethyl-8-azabicyclo[3.2.1]octan-3-yl]oxy}-1,2-diazin-3-yl)-2,3-dihydro-1H-pyrrolo[4,3-c]pyridin-1-one (248 mg, 83.90%) as a yellow solid.

¹H NMR (DMSO-d₆, 400 MHz) δ=8.98 (s, 1H), 8.71 (d, J=9.6 Hz, 1H), 8.37 (s, 1H), 8.23 (s, 1H), 7.88 (s, 1H), 7.37 (d, J=9.6 Hz, 1H), 5.51 (br t, J=5.7 Hz, 1H), 5.23 (s, 2H), 3.81 (s, 3H), 2.47-2.48 (s, 3H), 2.36 (br dd, J=13.0, 5.9 Hz, 2H), 2.06 (br d, J=7.8 Hz, 2H), 1.79 (br d, J=8.0 Hz, 2H), 1.69 (br t, J=11.9 Hz, 2H), 1.38 (s, 6H); LC/MS (ESI): 460.2 [M+H]⁺.

### Example 10. Synthesis of 6-(1,3-dimethyl-1H-pyrazol-4-yl)-2-(6-(((1S,3R,5R)-1-ethyl-5-methyl-8-azabicyclo[3.2.1]octan-3-yl)oxy)pyridazin-3-yl)-2,3-dihydro-1H-pyrrolo[3,4-c]pyridin-1-one (Compound No. 98)

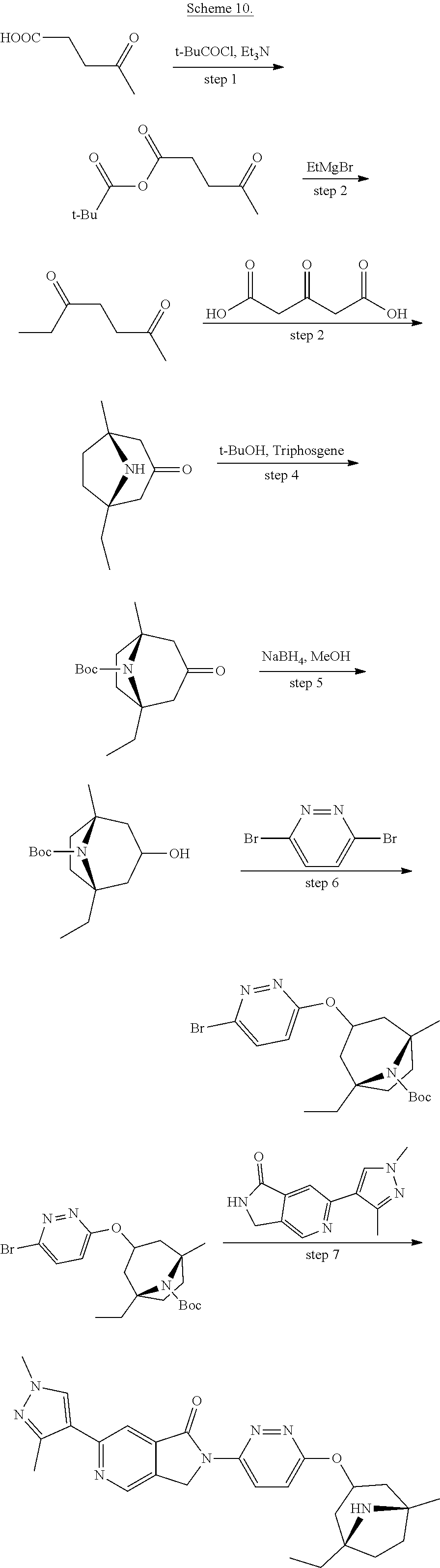

Step 1: A solution of pivaloyl chloride (41.96 mL, 341.03 mmol) in dry THF (50 mL) was added dropwise to a stirred and ice-cooled solution of 4-oxopentanoic acid (31.86 mL, 310.02 mmol) and triethylamine (128.92 mL, 930.07 mmol) in dry THF (300 mL) at 5° C. The solution was stirred for 1.5 h at 5° C., and the precipitated Et₃N·HCl was filtered off. The solid salt was washed with THF. The combined filtrate and washings were dried (MgSO₄), and concentrated in vacuo to give 2,2-dimethylpropanoic 4-oxopentanoic anhydride (57 g, 284.67 mmol, 91.82%) as a colorless oil which was used immediately in the next step. LC/MS (ESI): 201.1 [M+H]⁺.

Step 2: A solution of EtMgBr in THF (1 M, 323.14 mL, 313.14 mmol) was added dropwise over 30 min to a stirred and cooled solution of 2,2-dimethylpropanoic 4-oxopentanoic anhydride (57 g, 284.67 mmol) in dry THF (300 mL) at −60° C. under N₂. After the addition, the mixture was stirred for 30 min at −60° C. Subsequently the cooling bath was removed and the mixture was stirred for an additional 1 h. The reaction was quenched with the addition of ice and NH₄Cl solution, and was then extracted with EA. The EA solution was washed successively with water and brine, dried over MgSO₄, and concentrated in vacuo. The residues obtained from two runs were combined and distilled in vacuo to give heptane-2,5-dione (25 g, 195.05 mmol, 68.52%) as a colorless oil. LC/MS (ESI): 129.1 [M+H]⁺.

Step 3: KOH (49.25 g, 877.74 mmol) in water (50 mL) was added to a vigorous stirred solution of heptane-2,5-dione (25 g, 195.05 mmol) and 3-oxopentanedioic acid (42.30 mL, 370.60 mmol) in water (250 mL) at 0° C. NH₄Cl (20.59 mL, 585.16 mmol) and KOAc (38.29 g, 390.11 mmol) in water (200 mL) were added at 5° C. The pH value was adjusted to ~9 by using solid KOH. The mixture was stirred at room temperature for 4 days during which time the pH value was adjusted to ~9 by using additional solid KOH. The solution was diluted with 300 mL of water and transferred to a 2 L beaker and cooled to 5° C. and acidified with pure sulfuric acid until pH=2. Gas evolution was observed during the acidification. The aqueous layer divided in two batches. Each batch was washed with EA (2×1000 mL) and basified with solid KOH until pH=10. The aqueous layer was extracted with 15% MeOH in DCM (4×1000 mL). The combined organic layers were washed with brine (1000 mL), dried over sodium sulfate, filtered, and concentrated in vacuo to afford (1S,5R)-1-ethyl-5-methyl-8-azabicyclo[3.2.1]octan-3-one (9 g, 53.81 mmol, 27.59%) as a dark brown oil. LC/MS (ESI): 169.1 [M+H]⁺.

Step 4: To an ice-cold solution of (1S,5R)-1-ethyl-5-methyl-8-azabicyclo[3.2.1]octan-3-one (9 g, 53.81 mmol) in DCM (80 mL) was added pyridine (10.86 mL, 134.53 mmol). Trichloromethyl [(trichloromethyl)oxy]methanoate (3.59 mL, 21.52 mmol) was dissolved in DCM (20 mL) and added dropwise to the reaction mixture. After 30 minutes, 2-methylpropan-2-ol (10.23 mL, 107.62 mmol) was added and the mixture was gradually warmed to RT and stirred for 8 h. The reaction was quenched with water and diluted with DCM (300 mL). The mixture was washed with 1N HCl (1×50 mL), saturated sodium bicarbonate (1×100 mL) and brine (1×100 mL). The organic layer was collected and dried over sodium sulfate and then concentrated in vacuo. The crude mixture was purified by silica gel column chromatography with PE/EA 20/1, v/v to provided 2-methylpropan-2-yl (1R)-5-ethyl-1-methyl-3-oxo-8-azabicyclo[3.2.1]octane-8-carboxylate (7.5 g, 28.05 mmol, 52.13%) as a sightly yellow oil. LC/MS (ESI): 268.2 [M+H]⁺.

Step 5: 2-Methylpropan-2-yl (1R)-5-ethyl-1-methyl-3-oxo-8-azabicyclo[3.2.1]octane-8-carboxylate (7.5 g, 28.05 mmol) was dissolved in EtOH (60 mL) and the solution was heated to reflux. NaBH₄ (0.31 mL, 8.42 mmol) was added and the mixture was stirred for 15 min. The mixture was cooled to RT and quenched with aqueous ammonium chloride. The organic solvent was removed under vacuum and the residue was redissolved in EA (200 mL). The mixture was washed with brine, then the organic layer was collected and dried over sodium sulfate and then concentrated in vacuo. The residue was purified by silica gel column chromatography with PE/EtOAc to provided 2-methylpropan-2-yl (1R)-5-ethyl-3-hydroxy-1-methyl-8-azabicyclo[3.2.1]octane-8-carboxylate (3.7 g, 13.74 mmol, 48.97%). LC/MS (ESI): 270.2 [M+H]⁺.

Step 6: To a solution of 2-methylpropan-2-yl (1R)-5-ethyl-3-hydroxy-1-methyl-8-azabicyclo[3.2.1]octane-8-carboxylate (3.7 g, 13.74 mmol) in anhydrous THF (35 mL) was added NaH (0.33 g, 13.74 mmol). The solution was stirred at room temperature for 30 min, then 3,6-dibromo-1,2-diazine (3.27 g, 13.74 mmol) was added and the mixture was stirred at RT for 8 h. The reaction mixture was quenched with saturated ammonium chloride and diluted with EA (200 mL). The mixture was washed with brine, the organic layer was collected and dried over sodium sulfate and then concentrated in vacuo. The crude was purified by silica gel column chromatography with PE/EA 20/1, v/v to provided 2-methylpropan-2-yl (1R)-3-[(6-bromo-1,2-diazin-3-yl)oxy]-5-ethyl-1-methyl-8-azabicyclo[3.2.1]octane-8-carboxylate (2 g, 4.69 mmol, 34.15%) as a white solid. LC/MS (ESI): 426.1 [M+H]⁺.

Step 7: To a round bottom flask charged with 2-methylpropan-2-yl (1S)-3-[(6-bromo-1,2-diazin-3-yl)oxy]-1-ethyl-5-methyl-8-azabicyclo[3.2.1]octane-8-carboxylate (150 mg, 0.35 mmol), 6-(1,3-dimethylpyrazol-4-yl)-2,3-dihydro-1H-pyrrolo[4,3-c]pyridin-1-one (88.33 mg, 0.39 mmol), Cs₂CO₃ (102.23 mg, 0.31 mmol, and XantPhos Pd G4 (16.94 mg, 0.01 mmol) was added 1,4-dioxane (4 mL). The reaction mixture was degassed with argon for 5 min and then heated at 90° C. for 8 h. The reaction mixture was cooled to RT and diluted with EA. The organic phase was washed with brine, dried over sodium sulfate, and concentrated under vacuum. The crude was purified by silica gel column chromatography with DCM/MeOH 20/1, v/v to afford 2-methylpropan-2-yl (1S,5R)-3-({6-[6-(1,3-dimethylpyrazol-4-yl)-1-oxo-2,3-dihydro-1H-pyrrolo[4,3-c]pyridin-2-yl]-1,2-diazin-3-yl}oxy)-1-ethyl-5-methyl-8-azabicyclo[3.2.1]octane-8-carboxylate (60 mg), which was dissolved HCl in dioxane (2 mL, 4.0 mol/L). The reaction was stirred at RT for 1 h and then concentrated. The residue was purified by RP HPLC using MeCN and H₂O to provide 6-(1,3-dimethylpyrazol-4-yl)-2-(6-{[(1S,5R)-1-ethyl-5-methyl-8-azabicyclo[3.2.1]octan-3-yl]oxy}-1,2-diazin-3-yl)-2,3-dihydro-1H-pyrrolo[4,3-c]pyridin-1-one (20 mg, 0.04 mmol, 12.00%) as a white solid.

¹H NMR (DMSO-d₆, 400 MHz) δ=8.98 (s, 1H), 8.70 (d, J=9.6 Hz, 1H), 8.37 (s, 1H), 8.28 (s, 1H), 7.88 (s, 1H), 7.34 (d, J=9.6 Hz, 1H), 5.51 (s, 1H), 5.24 (s, 2H), 3.81 (s, 3H), 2.48 (s, 3H), 2.26 (br dd, J=5.6, 12.4 Hz, 2H), 1.90 (br t, J=7.1 Hz, 2H), 1.71-1.48 (m, 6H), 1.30 (s, 3H), 0.91 (t, J=7.4 Hz, 3H); LC/MS (ESI): 474.2 [M+H]⁺.

### Example 11. Synthesis of 6-(1,3-dimethyl-1H-pyrazol-4-yl)-2-(6-((5,5-dimethyl-4-azaspiro[2.5]octan-7-yl)oxy)pyridazin-3-yl)-2,3-dihydro-1H-pyrrolo[3,4-c]pyridin-1-one (Compound No. 59)

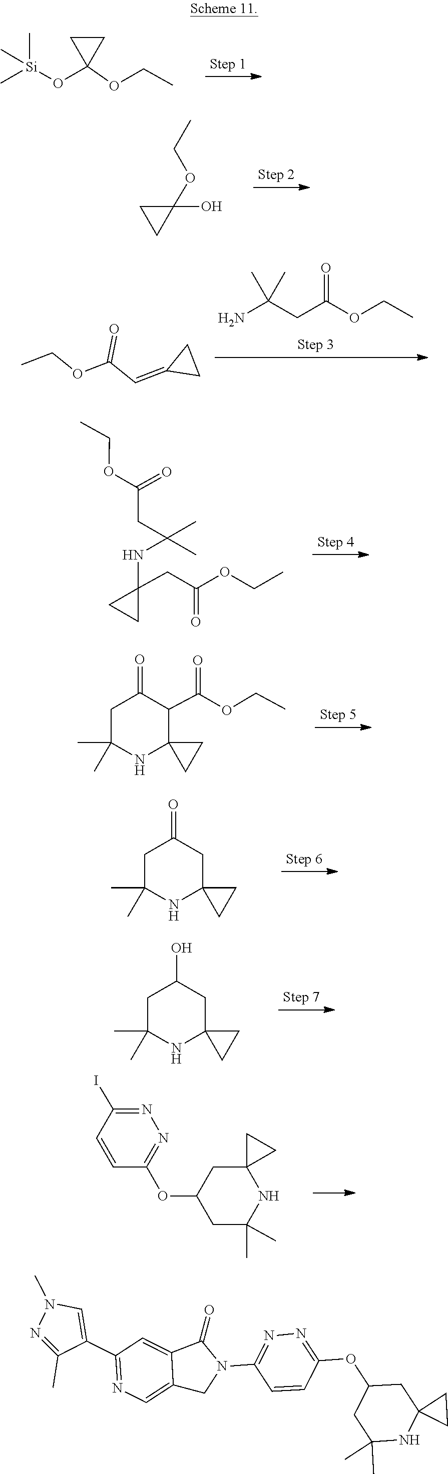

Step 1: A solution of 1-ethoxy-1-[(trimethylsilyl)oxy]cyclopropane (11.5 mL, 57.37 mmol, 1.0 eq) in MeOH (100 mL) was stirred at RT overnight. The reaction mixture was concentrated under vacuum and use directly in the next step without purification.

Step 2: A solution of 1-ethoxycyclopropan-1-ol (5.9 g, 57.38 mmol, 1.0 eq) and benzoic acid (1.4 g, 11.48 mmol, 0.2 eq) in toluene (6 mL) was stirred at 110° C. for 15 min. Then ethyl (triphenyl-λ5-phosphanylidene)acetate (20.0 g, 57.38 mmol, 1.0 eq) was added and the mixture was stirred at 110° C. for 48 hours. The reaction mixture was concentrated under vacuum. The crude product was purified by silica gel chromatography eluted with PE to give ethyl cyclopropylideneacetate (3.1 g, 42.4%).

Step 3: A solution of ethyl cyclopropylideneacetate (1.7 g, 13.47 mmol, 1.0 eq), ethyl 3-amino-3-methylbutanoate (2.3 g, 16.17 mmol, 1.2 eq) in DIEA (2.99 mL, 18.06 mmol, 1.34 eq) was stirred at 60° C. for 3 h. The reaction was monitored by TLC and LC/MS. The crude product was purified by silica gel chromatography eluted with PE:EA (5:1) to obtain ethyl 3-((1-(2-ethoxy-2-oxoethyl)cyclopropyl)amino)-3-methylbutanoate (1.2 g, 32.2%). LC/MS (ESI): 272.1 [M+H]⁺.

Step 4: To a solution of ethyl 3-((1-(2-ethoxy-2-oxoethyl)cyclopropyl)amino)-3-methylbutanoate (980 mg, 3.64 mmol, 1.0 eq) in THF (10 mL) was added t-BuOK (5.46 mL, 5.46 mmol) at 0° C. under N₂ atmosphere. The reaction mixture was stirred at 0° C. for 1 h. LC/MS showed the desired mass. The reaction mixture was poured into saturated aqueous NH₄Cl (10 mL). The resulting mixture was extracted with EA (2×20 mL). The organic layer was dried over Na₂SO₄ and evaporated to dryness. The crude product was purified by silica gel chromatography eluted with PE/EA (3:1) to obtain ethyl 5,5-dimethyl-7-oxo-4-azaspiro[2.5]octane-8-carboxylate (618 mg, 76.1%). LC/MS (ESI): 226.1 [M+H]⁺.

Step 5: A solution of ethyl 7-oxospiro[4-azaspiro[2.5]octane-5,1′-cyclopropane]-8-carboxylate (618.0 mg, 2.77 mmol, 1.0 eq) in a mixture of H₂O (5 mL) and H₂SO₄ (5 mL) was stirred at 120° C. for 1 h. LC/MS showed desired mass. The reaction mixture was poured into ice water. The aqueous layer was adjusted to pH over 9 and extracted with EA (3×20 mL). The crude was concentrated under reduced pressure to yield 5,5-dimethyl-4-azaspiro[2.5]octan-7-one (326.0 mg, 77.9%). LC/MS (ESI): 154.1 [M+H]⁺.

Step 6: To a solution of spiro[4-azaspiro[2.5]octane-5,1′-cyclopropane]-7-one (86.0 mg, 0.57 mmol, 1.0 eq) in MeOH (5 mL) was added NaBH₄ (64.5 mg, 1.71 mmol, 3.0 eq) at 0° C. The mixture was stirred at RT for 1 h. LC/MS showed desired mass. The reaction mixture was diluted with H₂O and the pH was adjusted to 7. The aqueous layer was extracted with EA (2×10 mL) and (DCM/propan-2-ol:IPA, 10:1). The organic layer was dried over Na₂SO₄ and evaporated to dryness. The crude product was use directly in the next step without further purification. LC/MS (ESI): 156.1 [M+H]⁺.

Step 7: To a solution of 5,5-dimethyl-4-azaspiro[2.5]octan-7-ol (106.0 mg, 0.69 mmol, 1.0 eq) in THF (5 mL) was added NaH (39.0 mg, 0.98 mmol, 1.4 eq) at 0° C. under N₂ atmosphere. The reaction mixture was stirred at 0° C. under N₂ atmosphere for 20 min. Then, 3,6-diiodo-1,2-diazine (252.5 mg, 0.76 mmol, 1.1 eq) was added the mixture was stirred at 60° C. overnight. The reaction was cooled to RT and diluted with DMF (5 mL) and then the reaction was stirred for another 3 h at 100° C. LC/MS showed desired mass. The reaction was cooled to RT and then pouring into water. The aqueous layer was extracted with EA (3×10 mL). The organic layer was washed with brine, dried over Na₂SO₄ and evaporated to dryness. The crude product was purified by silica gel chromatography eluted with PE: EA (1:1) to obtain 6-(1,3-dimethyl-1H-pyrazol-4-yl)-2-(6-((5,5-dimethyl-4-azaspiro[2.5]octan-7-yl)oxy)pyridazin-3-yl)-2,3-dihydro-1H-pyrrolo[3,4-c]pyridin-1-one (146.0 mg, 59.1%). LC/MS (ESI): 360.0 [M+H]⁺.

Step 8: A solution of 7-((6-iodopyridazin-3-yl)oxy)-5,5-dimethyl-4-azaspiro[2.5]octane (48.0 mg, 0.13 mmol, 1.0 eq), 3-(1,3-dimethylpyrazol-4-yl)-6,7-dihydro-5H-pyrrolo[4,3-b]pyridin-5-one (32.2 mg, 0.14 mmol, 1.05 eq), (1R,2R)-cyclohexane-1,2-diamine (1.5 mg, 0.01 mmol, 0.1 eq), CuI (2.7 mg, 0.01 mmol, 0.1 eq), K₂CO₃ (55.7 mg, 0.40 mmol, 3.0 eq) in DMF (3 mL) was stirred at 120° C. overnight under N₂ atmosphere. LC/MS showed desired mass and reactant consumed. The reaction mixture was evaporated to dryness. The crude product was purified by silica gel chromatography eluted with DCM/MeOH (10:1) then prep-HPLC(FA) to obtain 3-(1,3-dimethylpyrazol-4-yl)-6-[6-(spiro[4-azaspiro[2.5]octane-5,1′-cyclopropane]-7-yloxy)-1,2-diazin-3-yl]-6,7-dihydro-5H-pyrrolo[4,3-b]pyridin-5-one (12.92 mg, 19.1%).

¹H NMR (DMSO-d₆, 400 MHz) δ=8.98 (s, 1H), 8.69 (d, J=9.6 Hz, 1H), 8.37 (s, 1H), 8.19 (br s, 1H), 7.88 (s, 1H), 7.32 (d, J=9.6 Hz, 1H), 5.63-5.53 (m, 1H), 5.24 (s, 2H), 3.81 (s, 3H), 2.48 (s, 3H), 2.11 (br dd, J=3.6, 12.3 Hz, 1H), 1.88-1.80 (m, 1H), 1.63 (br dd, J=4.3, 11.8 Hz, 1H), 1.37 (t, J=11.6 Hz, 1H), 1.21 (s, 3H), 1.10 (s, 3H), 0.59-0.52 (m, 2H), 0.51-0.44 (m, 1H), 0.40-0.33 (m, 1H); LC/MS (ESI): 460.1 [M+H]⁺.

### Example 12. Synthesis of 2-(6-(((2S,6S)-2,6-dicyclopropylpiperidin-4-yl)oxy)pyridazin-3-yl)-6-(1,3-dimethyl-1H-pyrazol-4-yl)-2,3-dihydro-1H-pyrrolo[3,4-c]pyridin-1-one (Compound No. 104)

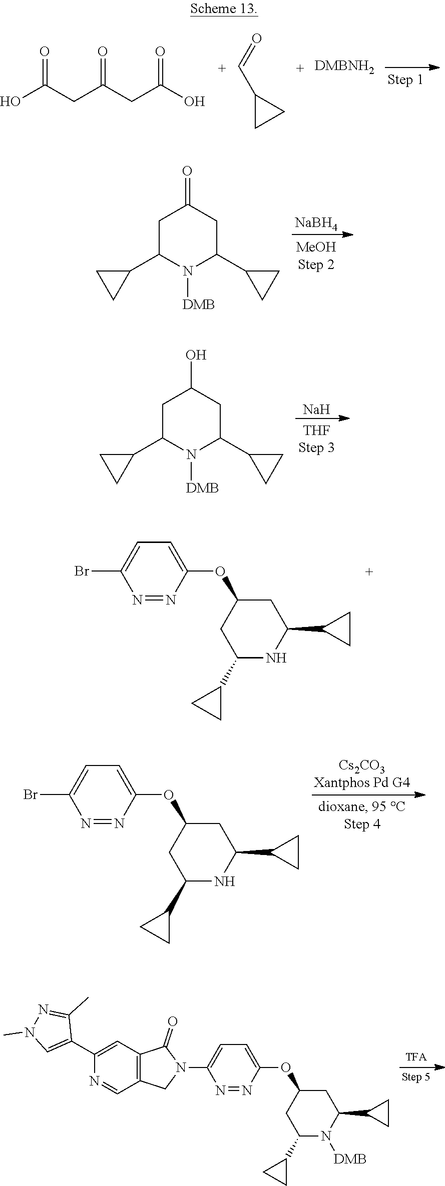

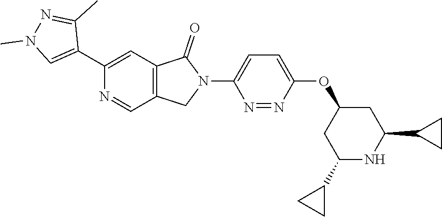

Step 1: To a solution of acetone dicarboxylic acid (3.0 g, 0.5 mmol) in water (20 mL) was added cyclopropanecarbaldehyde (2.876 g, 41 mmol) and then (2,4-dimethoxyphenyl)methanamine (2.8 g, 20.5 mmol) was added portionwise over 10 min. The resulting reaction was stirred at room temperature for 3 days. The reaction mixture was extracted with DCM (3×60 mL) and the combined extracts were washed with brine, dried over anhydrous Na₂SO₄, and dried under vacuum to give a brown residue. The isomeric piperidones were separated by silica gel chromatography with hexane/ethyl acetate (7/1, v/v). The desired compound (3.4 g, 54%) was obtained as a pale yellow oil.

Step 2: To a mixture of 2,6-dicyclopropyl-1-(3,4-dimethylbenzyl)piperidin-4-one (5.0 g, 15.18 mmol) in MeOH (50 mL) was added NaBH₄ (0.57 g, 15.18 mmol) at RT under N₂ and the mixture was stirred at for 2 h. The mixture was concentrated and the residue was purification by chromatography on silica gel DCM:MeOH=10:1 to give 2,6-dicyclopropyl-1-(3,4-dimethylbenzyl)piperidin-4-ol (2.0 g, 39.76%) as an oil.

Step 3: To a mixture of 2,6-dicyclopropyl-1-(3,4-dimethylbenzyl)piperidin-4-ol (2.0 g, 6.03 mmol) in THF (30 mL) was added NaH (0.48 g, 12.07 mmol) at RT under N₂ and the mixture was stirred for 1 h. Then 3,6-dibromo-1,2-diazine (1.87 g, 7.84 mmol) was added and the mixture was stirred at 50° C. for 16 h. The mixture was quenched with MeOH and concentrated under vacuum. The resulting residue was purification by chromatography on silica gel PE:EA=10:1-5:1 to give 3-bromo-6-(((2S,6S)-2,6-dicyclopropylpiperidin-4-yl)oxy)pyridazine (440, 14.93%) and 3-bromo-6-(((2R,4s,6S)-2,6-dicyclopropylpiperidin-4-yl)oxy)pyridazine (800 mg, 1.64 mmol, 27.16%) as a white solid.

Step 4: To a mixture of 6-(1,3-dimethylpyrazol-4-yl)-2,3-dihydro-1H-pyrrolo[4,3-c]pyridin-3-one (70 mg, 0.31 mmol) and 3-bromo-6-(((2S,6S)-2,6-dicyclopropylpiperidin-4-yl)oxy)pyridazine (149.79 mg, 0.31 mmol) in dioxane (10 mL) was added XantPhos Pd G4 (49.68 mg, 0.03 mmol) and Cs₂CO₃ (299.77 mg, 0.92 mmol) at RT under N₂ and the mixture was stirred at 95° C. for 3 h. The mixture was concentrated under reduced pressure and the residue was purification by chromatography on silica gel DCM:MeOH=10 to give 2-(6-(((2S,6S)-2,6-dicyclopropyl-1-(3,4-dimethylbenzyl)piperidin-4-yl)oxy)pyridazin-3-yl)-6-(1,3-dimethyl-1H-pyrazol-4-yl)-2,3-dihydro-1H-pyrrolo[3,4-c]pyridin-1-one (170 mg, 86.36%) as a yellow solid.

Step 5: A mixture of 2-(6-(((2S,6S)-2,6-dicyclopropyl-1-(3,4-dimethylbenzyl)piperidin-4-yl)oxy)pyridazin-3-yl)-6-(1,3-dimethyl-1H-pyrazol-4-yl)-2,3-dihydro-1H-pyrrolo

[3,4-c]pyridin-1-one (170 mg, 0.27 mmol) in TFA (20 mL) was stirred at 110° C. for 48 h. After the reaction was completed, the mixture was concentrated under reduced pressure and the residue was purified by pre-HPLC(TFA) to give 6-(1,3-dimethylpyrazol-4-yl)-2-(6-{[(2S,6S)-2,6-dicyclopropylhexahydropyridin-4-yl]oxy}-1,2-diazin-3-yl)-2,3-dihydro-1H-pyrrolo[4,3-c]pyridin-1-one (78.73 mg, 60.56%) as a yellow solid.

¹H NMR (DMSO-d_{b}) δ=8.98 (s, 1H), 8.73 (d, J=9.6 Hz, 1H), 8.34 (s, 1H), 7.88 (s, 1H), 7.38 (d, J=9.6 Hz, 1H), 5.64 (dt, J=9.0, 4.7 Hz, 1H), 5.25 (s, 2H), 3.82 (s, 3H), 2.90-3.00 (m, 2H), 2.46-2.49 (m, 3H), 2.40-2.46 (m, 1H), 2.29-2.38 (m, 1H), 1.85-2.06 (m, 2H), 1.24-1.34 (m, 1H), 1.18 (td, J=8.5, 4.0 Hz, 1H), 0.53-0.70 (m, 5H), 0.35-0.51 (m, 3H), 1H not observed (NH); LC/MS (ESI): 486.2 [M+H]⁺.

### Example 13. Synthesis of 3-(2,5-dimethyl-2H-1,2,3-triazol-4-yl)-6-(6-(((1R,3s,5S)-1,5-dimethyl-8-azabicyclo[3.2.1]octan-3-yl)oxy)pyridazin-3-yl)-6,7-dihydro-5H-pyrrolo[3,4-b]pyridin-5-one (Compound No. 206)

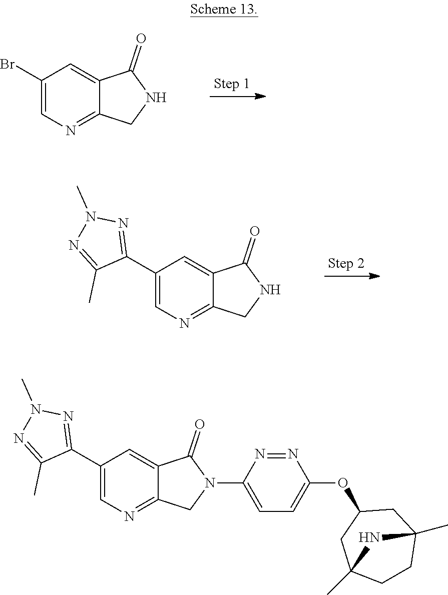 [SMILES: O=C1NCc2ncc(Br)cc21 ; Cc1nn(C)nc1-c1cnc2c(c1)C(=O)NC2 ; Cc1nn(C)nc1-c1cnc2c(c1)C(=O)N(c1ccc(O[C@H]3C[C@]4(C)CC[C@](C)(C3)N4)nn1)C2]

Step 1: 4-Bromo-2,5-dimethyl-2H-1,2,3-triazole (262.0 mg, 1.5 mmol, 1.5 eq); 4,4,4′,4′,5,5,5′,5′-octamethyl-2,2′-bi(1,3,2-dioxaborolane (762 mg, 3.0 mmol, 3.0 eq), potassium acetate (300 mg, 3.0 mmol, 3.0 eq) and PdCl₂(dppf) were added to the CH₂Cl₂ adduct (109.3 mg, 0.15 mmol, 0.1 eq) and the solids were degassed under N₂. Anhydrous dioxane (9 mL) was added and the mixture was heated to 90° C. After the reaction was completed, the reaction mixture was cooled to room temperature. Next 3-bromo-6,7-dihydro-5H-pyrrolo[3,4-b]pyridin-5-one (211.9 mg, 1.0 mmol, 1.0 eq), aqueous potassium carbonate (2 M, 2 mL) and PdCl₂(dppf) were added and the mixture was heated to 90° C. After the reaction was competed, the mixture was cooled, filtered through Celite, and washed with EA (100 mL). The filtrate was washed with brine and then the organic layer was collected, dried over sodium sulfate, and then concentrated under vacuo. The crude mixture was purified by silica gel column chromatography with DCM/MeOH 20/1, v/v to obtain 3-(2,5-dimethyl-2H-1,2,3-triazol-4-yl)-6,7-dihydro-5H-pyrrolo[3,4-b]pyridin-5-one (116 mg, 51%) as a brown solid. LC/MS (ESI): 230.1 [M+H]⁺.

Step 2: To a round bottom flask charged with 3-(2,5-dimethyl-2H-1,2,3-triazol-4-yl)-6,7-dihydro-5H-pyrrolo[3,4-b]pyridin-5-one (37.4 mg, 0.15 mmol, 1.0 eq), tert-butyl (1R,3s,5S)-3-((6-bromopyridazin-3-yl)oxy)-1,5-dimethyl-8-azabicyclo[3.2.1]octane-8-carboxylate (61.8 mg, 0.15 mmol, 1.1 eq), Cs₂CO₃ (148.9 mg, 0.46 mmol, 3.0 eq), and XantPhos Pd G4 (14.2 mg, 0.015 mmol, 0.1 eq) 1,4-dioxane (4 mL) was added and the reaction mixture was degassed with argon for 5 min and then heated to 90° C. for 8 h. The reaction mixture was cooled to RT and diluted with EA. The organic phase was washed with brine, dried over sodium sulfate, and concentrated under vacuum. The crude product was purified by silica gel column chromatography with DCM/MeOH 20/1, v/v and was then dissolved HCl in dioxane (2 mL, 4.0 mol/L) and stirred for 1 h at RT. The product was concentrated under reduced pressure and the resulting residue was purified by RP HPLC using MeCN and H₂O as eluent to provide 3-(2,5-dimethyl-2H-1,2,3-triazol-4-yl)-6-(6-(((1R,3s,5S)-1,5-dimethyl-8-azabicyclo[3.2.1]octan-3-yl)oxy)pyridazin-3-yl)-6,7-dihydro-5H-pyrrolo[3,4-b]pyridin-5-one (37.8 mg, 54.7%) as a white solid.

¹H NMR (DMSO-d₆, 400 MHz) δ=9.18 (d, J=2.0 Hz, 1H), 8.87 (br d, J=10.6 Hz, 1H), 8.79 (br s, 1H), 8.75 (d, J=9.6 Hz, 1H), 8.39 (d, J=2.0 Hz, 1H), 7.41 (d, J=9.5 Hz, 1H), 5.55 (tt, J=10.9, 5.6 Hz, 1H), 5.24 (s, 2H), 4.19 (s, 3H), 2.46 (br d, J=6.1 Hz, 2H), 2.18 (br d, J=8.0 Hz, 2H), 1.90 (br d, J=8.3 Hz, 2H), 1.80 (br t, J=12.1 Hz, 2H), 1.46 (s, 6H), 3H not observed (triazole methyl overlapped with DMSO-d6 residue); LC/MS (ESI): 461.2 [M+H]⁺.

### Example 14. Synthesis of 3-(3-fluoro-1-methyl-1H-pyrazol-4-yl)-6-(6-((2,2,6,6-tetramethylpiperidin-4-yl)oxy)pyridazin-3-yl)-6,7-dihydro-5H-pyrrolo[3,4-b]pyridin-5-one (Compound No. 190)

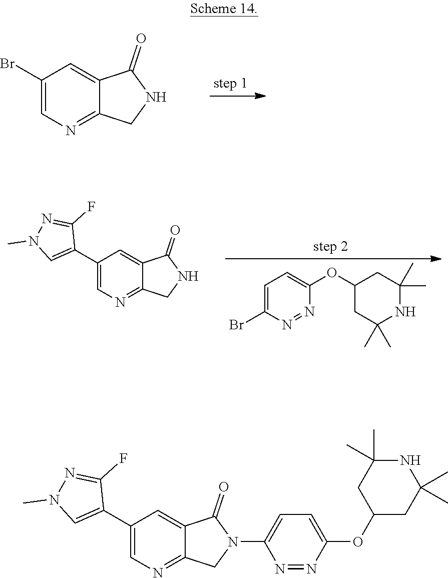

Step 1: 4-Bromo-2,5-dimethyl-2H-1,2,3-triazole (262.0 mg, 1.5 mmol, 1.5 eq), 4,4,4′,4′,5,5,5′,5′-octamethyl-2,2′-bi(1,3,2-dioxaborolane (762 mg, 3.0 mmol, 3.0 eq), potassium acetate (300 mg, 3.0 mmol, 3.0 eq) and PdCl₂(dppf)·CH₂Cl₂ adduct (109.3 mg, 0.15 mmol, 0.1 eq) were charged to a three-neck 100 mL round bottom flask under nitrogen. Anhydrous dioxane (9 mL) was added and the mixture was heated to 90° C. When full conversion was achieved, the reaction mixture was cooled to room temperature. 4-Bromo-3-fluoro-1-methyl-1H-pyrazole (177.9 mg, 1.0 mmol, 1.0 eq), aqueous potassium carbonate (2 M, 2 mL) and PdCl₂(dppf)·CH₂Cl₂ adduct (109.3 mg, 0.15 mmol, 0.1 eq) were added and the mixture was heated to 90° C. When full conversion was achieved, the mixture was cooled, filtered through a pad of Celite, and the filter cake was washed with EA (100 mL). The filtrate was washed with brine and the organic layer was collected, dried over sodium sulfate, and concentrated in vacuo. The crude product was purified by silica gel column chromatography with DCM/MeOH 20/1, v/v to obtain 3-(3-fluoro-1-methyl-1H-pyrazol-4-yl)-6,7-dihydro-5H-pyrrolo[3,4-b]pyridin-5-one (116 mg, 51%) as a brown solid. LC/MS (ESI): 233.1 [M+H]⁺.

Step 2: 3-[1-(Trifluoromethyl)pyrazol-4-yl]-6,7-dihydro-5H-pyrrolo[4,3-b]pyridin-5-one (106 mg, 0.46 mmol), 3-bromo-6-((2,2,6,6-tetramethylpiperidin-4-yl)oxy)pyridazine (149.39 mg, 0.46 mmol), XantPhos Pd G4 (73.95 mg, 0.05 mmol) and Cs₂CO₃ (446.18 mg, 1.37 mmol) was dissolved in dioxane (8 mL) and the reaction mixture was stirred at 90° C. overnight. After the reactants was consumed, the mixture was cooled to room temperature, quenched with water and diluted with EA (100 mL). The mixture was washed with brine, and the organic layer was collected, dried over sodium sulfate, and concentrated in vacuo. The crude product was purified by silica gel column chromatography with DCM/MeOH to provided 3-(3-fluoro-1-methyl-1H-pyrazol-4-yl)-6-(6-((2,2,6,6-tetramethylpiperidin-4-yl)oxy)pyridazin-3-yl)-6,7-dihydro-5H-pyrrolo[3,4-b]pyridin-5-one (24.1 mg, 0.05 mmol, 10.91%) as a white solid.

¹H NMR (DMSO-d₆, 400 MHz) δ=8.99-9.09 (m, 1H), 8.68 (d, J=9.6 Hz, 1H), 8.37-8.47 (m, 1H), 8.22-8.31 (m, 1H), 7.24-7.36 (m, 1H), 5.57-5.68 (m, 1H), 5.19 (s, 2H), 3.80 (s, 3H), 2.05-2.07 (m, 2H), 1.24-1.29 (m, 2H), 1.23 (s, 6H), 1.08-1.13 (m, 6H), 1H not observed (NH); LC/MS (ESI): 466.2 [M+H]⁺.

### Example 15. Synthesis of 3-(1-ethyl-3-(trifluoromethyl)-1H-pyrazol-4-yl)-6-(6-((2,2,6,6-tetramethylpiperidin-4-yl)oxy)pyridazin-3-yl)-6,7-dihydro-5H-pyrrolo[3,4-b]pyridin-5-one (Compound No. 172)

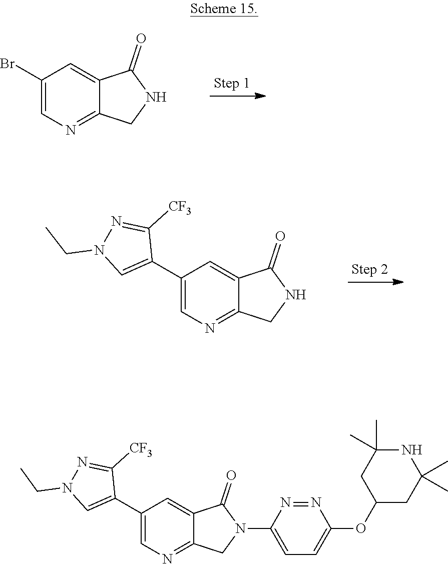 [SMILES: O=C1NCc2ncc(Br)cc21 ; CCn1cc(-c2cnc3c(c2)C(=O)NC3)c(C(F)(F)F)n1 ; CCn1cc(-c2cnc3c(c2)C(=O)N(c2ccc(OC4CC(C)(C)NC(C)(C)C4)nn2)C3)c(C(F)(F)F)n1]

Step 1: 3-Bromo-6,7-dihydro-5H-pyrrolo[3,4-b]pyridin-5-one (213.0 mg, 1.0 mmol, 1.0 eq), (1-ethyl-3-(trifluoromethyl)-1H-pyrazol-4-yl)boronic acid (312. mg, 1.5 mmol, 1.5 eq), aqueous potassium carbonate (2 M, 2 mL) and PdCl₂(dppf)·CH₂Cl₂ adduct (73.2 mg, 0.1 mmol, 0.1 eq) were charged to a three-neck 100 mL round bottom flask under nitrogen. Anhydrous dioxane (9 mL) was added and the mixture was heated to 85° C. overnight. Then the mixture was cooled, filtered through a pad of Celite, and the filter cake was rinsed with EA (100 mL). The filtrate was washed with brine and the organic layer was collected, dried over sodium sulfate, and concentrated in vacuo. The crude product was purified by silica gel column chromatography with DCM/MeOH 20/1, v/v to obtain 3-(1-ethyl-3-(trifluoromethyl)-1H-pyrazol-4-yl)-6,7-dihydro-5H-pyrrolo[3,4-b]pyridin-5-one (183.2 mg, 62%) as a brown solid. LC/MS (ESI): 297.1 [M+H]⁺.

Step 2: To a round bottom flask charged with 3-(1-ethyl-3-(trifluoromethyl)-1H-pyrazol-4-yl)-6,7-dihydro-5H-pyrrolo[3,4-b]pyridin-5-one (44.4 mg, 0.15 mmol, 1.0 eq), tert-butyl (1R,3s,5S)-3-((6-bromopyridazin-3-yl)oxy)-1,5-dimethyl-8-azabicyclo[3.2.1]octane-8-carboxylate (61.8 mg, 0.15 mmol, 1.1 eq), Cs₂CO₃ (148.9 mg, 0.46 mmol, 3.0 eq), and XantPhos Pd G4 (14.2 mg, 0.015 mmol, 0.1 eq) was added 1,4-dioxane (4 mL). The reaction mixture was degassed with argon for 5 min and then heated to 90° C. for 8 h. The reaction mixture was cooled to RT and diluted with EA. The organic phase was washed with brine, dried over sodium sulfate, and concentrated under vacuum. The crude product was purified by silica gel column chromatography with DCM/MeOH 20/1, v/v. The residue was purified by RP HPLC using MeCN and H₂O as eluent to provide 3-(1-ethyl-3-(trifluoromethyl)-1H-pyrazol-4-yl)-6-(6-((2,2,6,6-tetramethylpiperidin-4-yl)oxy)pyridazin-3-yl)-6,7-dihydro-5H-pyrrolo[3,4-b]pyridin-5-one (42.35 mg, 53.3%) as a white solid.

¹H NMR (400 MHz, DMSO-d₆) δ=8.92 (d, 1H, J=2.0 Hz), 8.71 (d, 1H, J=9.6 Hz), 8.50 (s, 1H), 8.32 (s, 1H), 8.19 (d, 1H, J=2.0 Hz), 7.35 (d, 1H, J=9.6 Hz), 5.64 (s, 1H), 5.24 (s, 2H), 4.30 (q, 2H, J=7.3 Hz), 2.17 (dd, 2H, J=3.9, 12.4 Hz), 1.5-1.5 (m, 3H), 1.4-1.5 (m, 2H), 1.34 (s, 6H), 1.24 (s, 6H); LC/MS (ESI): 530.2 [M+H]⁺.

### Example 16. Synthesis of 3-(3-(difluoromethyl)-1-methyl-1H-pyrazol-4-yl)-6-(6-(methyl(2,2,6,6-tetramethylpiperidin-4-yl)amino)pyridazin-3-yl)-6,7-dihydro-5H-pyrrolo[3,4-b]pyridin-5-one (Compound No. 198)

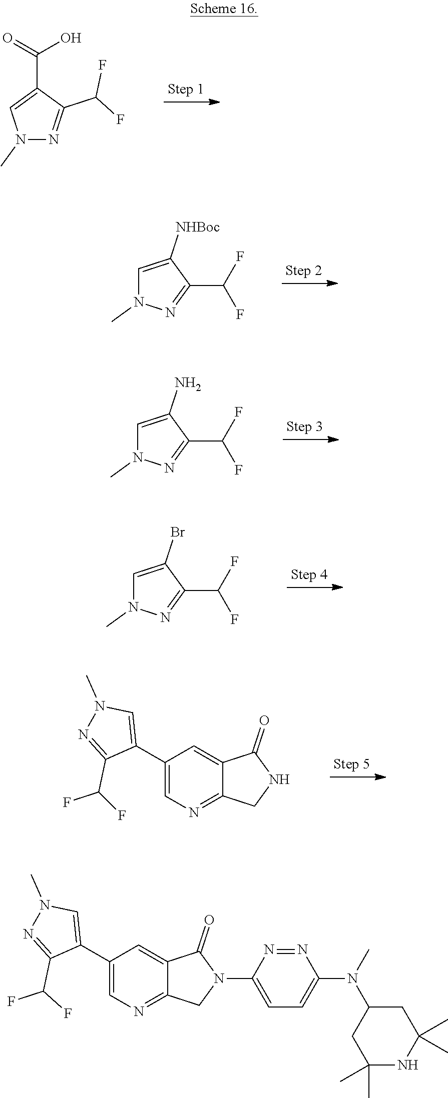

Step 1: To a solution of 3-(difluoromethyl)-1-methyl-1H-pyrazole-4-carboxylic acid (6.58 mL, 56.78 mmol) in 2-methylpropan-2-ol (200 mL) was added ethyl[di(prop-2-yl)]amine (18.82 mL, 113.56 mmol) and diphenyl azidophosphonate (15.99 mL, 73.81 mmol) and the reaction mixture was stirred at 90° C. for 4 h under N₂. The mixture was diluted with EA (10 mL*3) and H₂O (30 mL) and the organic layer was washed with brine (5 mL*3), dried over sodium sulfate, and then concentrated in vacuo. The crude product was purified by silica gel column chromatography with PE/EA 10/1, v/v to provided tert-butyl (3-(difluoromethyl)-1-methyl-1H-pyrazol-4-yl)carbamate (13.2 g, 53.39 mmol, 94.03%). LC/MS (ESI): 248.1 [M+H]⁺.

Step 2: To a solution of tert-butyl (3-(difluoromethyl)-1-methyl-1H-pyrazol-4-yl)carbamate (13.2 g, 53.39 mmol) in MeOH (30 mL) was added 4 μM HCl-MeOH (60 mL) and the reaction mixture was stirred at 25° C. overnight. Then the reaction mixture was concentrated to obtain the cured product 3-(difluoromethyl)-1-methyl-1H-pyrazol-4-amine (10.2 g) which was used in the next step without further purification. LC/MS (ESI): 148.1[M+H]⁺.

Step 3: To a solution of 3-(difluoromethyl)-1-methyl-1H-pyrazol-4-amine (3 g, 20.39 mmol) in acetonitrile (30 mL) was added 2-methyl-2-(nitrosooxidanyl)propane (3.67 mL, 30.59 mmol) and copper bromide (I) (1.24 mL, 40.78 mmol) and the reaction mixture was stirred at 70° C. for 1 h. The mixture was diluted with EA (50 mL*3) and H₂O (50 mL) and then the organic layer was washed with brine (30 mL*3), dried over sodium sulfate, and concentrated in vacuo. The crude product was purified by silica gel column chromatography with PE/EA (50/1-20/1-10/1, v/v) to provide 4-bromo-3-(difluoromethyl)-1-methyl-1H-pyrazole (2.3 g, 10.90 mmol, 53.46%). LC/MS (ESI): 210.9, 212.9 [M+H]⁺.

Step 4: 4-Bromo-3-(difluoromethyl)-1-methyl-1H-pyrazole (316.5 mg, 1.5 mmol, 1.5 eq), 4,4,4′,4′,5,5,5′,5′-octamethyl-2,2′-bi(1,3,2-dioxaborolane (762 mg, 3.0 mmol, 3.0 eq), potassium acetate (300 mg, 3.0 mmol, 3.0 eq) and PdCl₂(dppf)·CH₂Cl₂ adduct (109.3 mg, 0.15 mmol, 0.1 eq) were charged to a three-neck 100 mL round bottom flask under nitrogen. Anhydrous dioxane (9 mL) was added and the mixture was heated to 90° C. When full conversion was achieved, the reaction mixture was cooled to room temperature. Next 3-bromo-6,7-dihydro-5H-pyrrolo[3,4-b]pyridin-5-one (211.9 mg, 1.0 mmol, 1.0 eq), aqueous potassium carbonate (2 M, 2 mL) and PdCl₂(dppf)·CH₂Cl₂ adduct (109.3 mg, 0.15 mmol, 0.1 eq) were added and the mixture was heated to 90° C. When full conversion was achieved, the mixture was cooled, filtered through a pad of Celite, and the filter cake was rinsed with EA (100 mL). The filtrate was washed with brine and the organic layer was collected, dried over sodium sulfate, and concentrated in vacuo. The crude product was purified by silica gel column chromatography with DCM/MeOH 20/1, v/v to obtain 3-(3-(difluoromethyl)-1-methyl-1H-pyrazol-4-yl)-6,7-dihydro-5H-pyrrolo[3,4-b]pyridin-5-one (164.7 mg, 62%) as a brown solid. LC/MS (ESI): 265.1 [M+H]⁺.

Step 5: To a round bottom flask charged with 3-(3-(difluoromethyl)-1-methyl-1H-pyrazol-4-yl)-6,7-dihydro-5H-pyrrolo[3,4-b]pyridin-5-one (39.4 mg, 0.15 mmol, 1.0 eq), 6-bromo-N-methyl-N-(2,2,6,6-tetramethylpiperidin-4-yl)pyridazin-3-amine (61.8 mg, 0.15 mmol, 1.1 eq), Cs₂CO₃ (148.9 mg, 0.46 mmol, 3.0 eq), and XantPhos Pd G4 (14.2 mg, 0.015 mmol, 0.1 eq) was added 1,4-dioxane (4 mL). The reaction mixture was degassed with argon for 5 min and then heated at 90° C. for 8 h. The reaction mixture was cooled to RT and diluted with EA. The organic phase was washed with brine, dried over sodium sulfate, and concentrated under vacuum. The crude product was purified by silica gel column chromatography with DCM/MeOH 20/1, and then the residue was further purified by RP HPLC using MeCN and H₂O as eluent to provide 3-(3-(difluoromethyl)-1-methyl-1H-pyrazol-4-yl)-6-(6-(methyl(2,2,6,6-tetramethylpiperidin-4-yl)amino)pyridazin-3-yl)-6,7-dihydro-5H-pyrrolo[3,4-b]pyridin-5-one (3.83 mg, 5.0%) as a white solid.

¹H NMR (400 MHz, Methanol-d₄) δ: 8.97 (d, J=2.0 Hz, 1H), 8.63 (d, J=9.9 Hz, 1H), 8.34 (d, J=2.1 Hz, 1H), 8.12 (s, 1H), 7.29 (d, J=10.0 Hz, 1H), 7.03-6.74 (m, 1H), 5.22 (s, 2H), 4.00 (s, 3H), 2.99 (s, 3H), 1.83-1.69 (m, 4H), 1.50 (s, 6H), 1.35 (s, 6H); LC/MS (ESI): 511.2 [M+H]⁺.

### Example 17. Synthesis of (S)-6-(6-(3-cyclopropylpiperazin-1-yl)pyridazin-3-yl)-3-(1-methyl-1H-pyrazol-4-yl)-6,7-dihydro-5H-pyrrolo[3,4-b]pyridin-5-one (Compound No. 155)

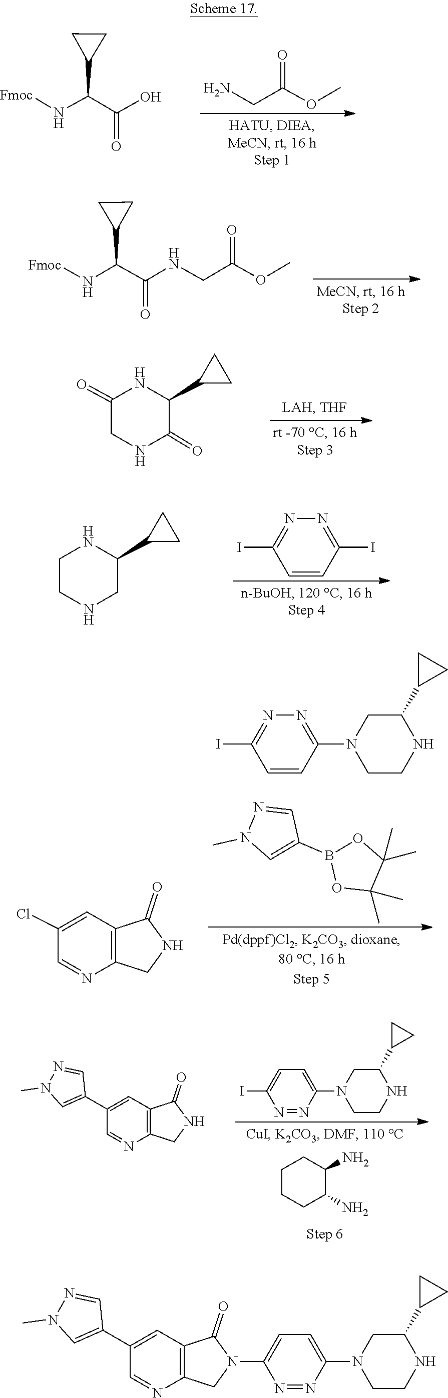

Step 1: To a solution of (S)-2-((((9H-fluoren-9-yl)methoxy)carbonyl)amino)-2-cyclopropylacetic acid (10 g, 28.29 mmol, 1.0 eq) and methyl aminoacetate (3.0 g, 33.95 mmol, 1.2 eq) in acetonitrile (50 mL) was added DIEA (9.1 g, 70.74 mmol, 2.5 eq) and HATU (11.8 g, 31.12 mmol, 1.1 eq), and the reaction mixture was stirred at RT for 16 h. Next the reaction mixture was filtered to provided methyl (S)-(2-((((9H-fluoren-9-yl)methoxy)carbonyl)amino)-2-cyclopropylacetyl)glycinate (9.5 g, 22.38 mmol, 79.09%) as a sightly yellow solid. LC/MS (ESI): 409.2 [M+H]⁺.

Step 2: A solution of methyl (S)-(2-((((9H-fluoren-9-yl)methoxy)carbonyl)amino)-2-cyclopropylacetyl)glycinate (5.0 g, 11.78 mmol, 1.0 eq) in acetonitrile (100 mL) was stirred RT for 16 h. After the reaction was completed, the solution was concentrated in vacuo to provided (S)-3-cyclopropylpiperazine-2,5-dione (4.85 g, 11.43 mmol, 97.00%) as a sightly yellow solid. LC/MS (ESI): 155.1 [M+H]⁺.

Step 3: LiAlH₄ (2.14 g, 56.40 mmol, 6.0 eq) was added to a vigorous stirred solution of (S)-3-cyclopropylpiperazine-2,5-dione (1.6 g, 9.40 mmol, 1.0 eq) in THF (20 mL) in an ice bath, and then the reaction mixture was stirred at 70° C. for 16 h. After the reaction was completed, the mixture was poured into KOH (6N) and stirred in an ice bath for 1 h. Next the mixture was filtered and the filtrate was extracted with EA, washed with brine, dried over Na₂SO₄, and concentrated in vacuo to provided (S)-2-cyclopropylpiperazine (1.2 g, 8.44 mmol, 89.74%) as a yellow oil. LC/MS (ESI): 127.1 [M+H]⁺.

Step 4: DIEA (1.13 g, 8.79 mmol, 1.46 mL, 2.5 eq) was added to a vigorous stirred solution of (2S)-2-(2-methylprop-2-yl)piperazine (0.5 g, 3.51 mmol, 1.0 eq) and 3,6-diiodo-1,2-diazine (1.17 g, 3.51 mmol, 1.0 eq) in n-BuOH (7 mL), and the reaction mixture was stirred at 120° C. for 16 h. After the reaction was competed, the mixture was concentrated in vacuo and the residue was diluted with EA, washed with water and brine, dried over Na₂SO₄, and concentrated in vacuo to provided (S)-3-(3-cyclopropylpiperazin-1-yl)-6-iodopyridazine (450 mg, 1.30 mmol, 36.98%) as a yellow solid. LC/MS (ESI): 331.1 [M+H]⁺.

Step 5: Pd(dppf)Cl₂ (49 mg, 0.06 mmol, 0.05 eq) was added to a vigorous stirred solution of 1-methyl-4-(4,4,5,5-tetramethyl-1,3,2-dioxaborolan-2-yl)pyrazole (250 mg, 1.20 mmol, 1.0 eq), 3-bromo-6,7-dihydro-5H-pyrrolo[4,3-b]pyridin-5-one (256 mg, 1.20 mmol, 1.0 eq) and K₂CO₃ (332 mg, 2.40 mmol, 2.0 eq) in dioxane (5 mL) and water (1 mL). The reaction mixture was stirred at 80° C. for 16 h. Next the reaction mixture was filtered and the filtrate was concentrated in vacuo. The crude product was purified by silica gel column chromatography with DCM/MeOH 10/1, v/v to give 3-(1-methylpyrazol-4-yl)-6,7-dihydro-5H-pyrrolo[4,3-b]pyridin-5-one (135 mg, 0.63 mmol, 52.51%) as a sightly yellow solid. LC/MS (ESI): 215.2 [M+H]⁺.

Step 6: 6-(1-Methylpyrazol-4-yl)-2,3-dihydro-1H-pyrrolo[4,3-c]pyridin-1-one (50 mg, 0.23 mmol, 1.0 eq) and (S)-3-(3-cyclopropylpiperazin-1-yl)-6-iodopyridazine (80 mg, 0.23 mmol, 1.0 eq) were dissolved in DMF (2 mL), and CuI (4 mg, 0.02 mmol, 0.10 eq), K₂CO₃ (97 mg, 0.70 mmol, 3.0 eq) and (1R,2R)-cyclohexane-1,2-diamine (2.7 mg, 0.02 mmol, 0.10 eq) were added and the reaction mixture was stirred at 100° C. for 16 h. Next the reaction was filtered and the filtrate was concentrated. The crude product was purified by pre-HPLC to provide (S)-6-(6-(3-cyclopropylpiperazin-1-yl)pyridazin-3-yl)-3-(1-methyl-1H-pyrazol-4-yl)-6,7-dihydro-5H-pyrrolo[3,4-b]pyridin-5-one (24.19 mg, 21.55%) as a white solid.

¹H NMR (DMSO-d₆, 400 MHz) δ=9.12 (d, J=1.6 Hz, 1H), 8.49 (d, J=9.8 Hz, 1H), 8.44 (s, 1H), 8.36 (d, J=1.8 Hz, 1H), 8.14 (s, 1H), 7.51 (d, J=10.0 Hz, 1H), 5.13 (s, 2H), 4.27 (br d, J=11.1 Hz, 1H), 4.15 (br d, J=10.8 Hz, 1H), 3.89 (s, 3H), 3.14-3.03 (m, 2H), 2.95 (br t, J=11.5 Hz, 1H), 2.84-2.74 (m, 2H), 0.84 (br d, J=7.1 Hz, 1H), 0.48 (br s, 2H), 0.33 (br s, 2H), 1H not observed (NH); LC/MS (ESI): 417.2 [M+H]⁺.

### Example 18. Synthesis of 6-(6-(((1R,3s,5S)-1,5-dimethyl-8-azabicyclo[3.2.1]octan-3-yl)oxy)pyridazin-3-yl)-3-(4-methylthiazol-5-yl)-6,7-dihydro-5H-pyrrolo[3,4-b]pyridin-5-one (Compound No. 248)

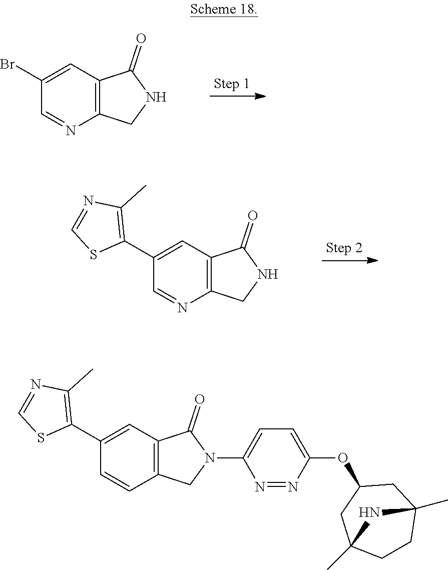 [SMILES: O=C1NCc2ncc(Br)cc21 ; Cc1ncsc1-c1cnc2c(c1)C(=O)NC2 ; Cc1ncsc1-c1ccc2c(c1)C(=O)N(c1ccc(O[C@H]3C[C@]4(C)CC[C@](C)(C3)N4)nn1)C2]

Step 1: 3-Bromo-6,7-dihydro-5H-pyrrolo[3,4-b]pyridin-5-one (213.0 mg, 1.0 mmol, 1.0 eq), 4-methyl-5-(4,4,5,5-tetramethyl-1,3,2-dioxaborolan-2-yl)thiazole (375 mg, 1.5 mmol, 1.5 eq), aqueous potassium carbonate (2 M, 2 mL) and PdCl₂(dppf)·CH₂Cl₂ adduct (73.2 mg, 0.1 mmol, 0.1 eq) were charged to a three-neck 100 mL round bottom flask under nitrogen. Anhydrous dioxane (9 mL) was added and the mixture was heated to 85° C. overnight. Then the mixture was cooled, filtered through a pad of Celite, and the filter cake was rinsed with EA (100 mL). The filtrate was washed with brine and the organic layer was collected, dried over sodium sulfate, and concentrated in vacuo. The crude product was purified by silica gel column chromatography with DCM/MeOH 20/1, v/v to obtain 3-(4-methylthiazol-5-yl)-6,7-dihydro-5H-pyrrolo[3,4-b]pyridin-5-one (143.2 mg, 62%) as brown solid. LC/MS (ESI): 232.1 [M+H]⁺.

Step 2: To a round bottom flask charged with 3-(4-methylthiazol-5-yl)-6,7-dihydro-5H-pyrrolo[3,4-b]pyridin-5-one (44.4 mg, 0.15 mmol, 1.0 eq), tert-butyl (1R,3s,5S)-3-((6-bromopyridazin-3-yl)oxy)-1,5-dimethyl-8-azabicyclo[3.2.1]octane-8-carboxylate (61.8 mg, 0.15 mmol, 1.1 eq), Cs₂CO₃ (148.9 mg, 0.46 mmol, 3.0 eq), and XantPhos Pd G4 (14.2 mg, 0.015 mmol, 0.1 eq) was added 1,4-dioxane (4 mL). The reaction mixture was degassed with argon for 5 min and was then heated to 90° C. for 8 h. The reaction mixture was cooled RT and diluted with EA. The organic phase was washed with brine, dried over sodium sulfate, and concentrated under vacuum. The crude product was purified by silica gel column chromatography with DCM/MeOH 20/1, v/v, and was then dissolved HCl in dioxane (2 mL, 4.0 mol/L). The reaction mixture was stirred at RT for 1 h, and was then concentrated. The resulting residue was purified by RP HPLC using MeCN and H₂O as eluent to provide 6-(6-(((1R,3s,5S)-1,5-dimethyl-8-azabicyclo[3.2.1]octan-3-yl)oxy)pyridazin-3-yl)-3-(4-methylthiazol-5-yl)-6,7-dihydro-5H-pyrrolo[3,4-b]pyridin-5-one (20.37 mg, 29.4%) as a white solid.

¹HNMR (400 MHz, DMSO-d₆) δ=9.15 (s, 1H), 9.00 (s, 1H), 8.72 (br d, J=9.5 Hz, 1H), 8.29 (br d, J=12.3 Hz, 3H), 7.38 (br d, J=9.5 Hz, 1H), 5.56-5.48 (m, 1H), 5.24 (s, 2H), 2.53-2.51 (m, 3H), 2.35 (br dd, J=5.3, 12.4 Hz, 2H), 2.06 (br d, J=7.5 Hz, 2H), 1.88-1.73 (m, 4H), 1.41 (br s, 6H); LC/MS (ESI): 463.2 [M+H]⁺.

### Example 19. Synthesis of 3-(1,3-dimethyl-1H-pyrazol-4-yl)-6-(6-((6,6-dimethyl-5-azaspiro[3.5]nonan-8-yl)oxy)pyridazin-3-yl)-6,7-dihydro-5H-pyrrolo[3,4-b]pyridin-5-one (Compound No. 249)

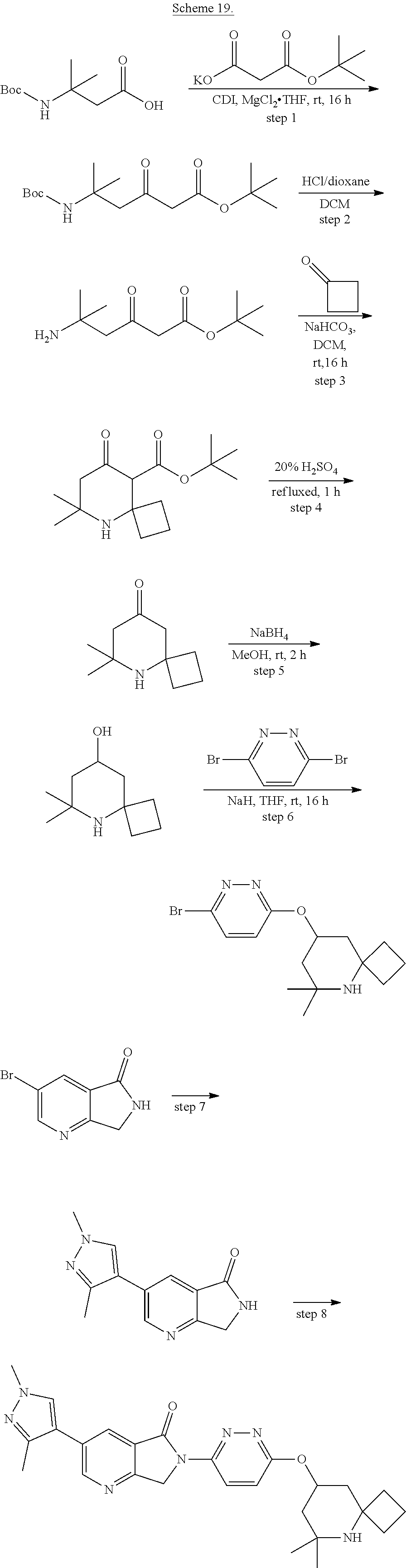

Step 1: To a solution of 3-((tert-butoxycarbonyl)amino)-3-methylbutanoic acid (12.0 g, 55.23 mmol, 1.0 eq) in THF(100 mL) was added CDI (10.75 g, 66.28 mmol, 1.20 eq) and the reaction mixture was stirred at RT for 1 h. Then added MgCl₂ (2.62 g, 43.08 mmol, 0.78 eq) and potassium 3-(tert-butoxy)-3-oxopropanoate (16.43 g, 82.85 mmol, 1.5 eq) were added and the reaction mixture was stirred at 50° C. overnight. Next the reaction mixture was concentrated and then diluted with H₂O (40 mL). The aqueous layer was extracted with EA (3×50 mL). The organic layer was dried over Na₂SO₄ and concentrated under reduced pressure. The crude product was purified by silica gel chromatography eluted with PE: EA (10:1) to give tert-butyl 5-((tert-butoxycarbonyl)amino)-5-methyl-3-oxohexanoate(10.5 g, 20.27%). LC/MS (ESI): 260.2 [M+H]⁺.

Step 2: To a solution of tert-butyl 5-((tert-butoxycarbonyl)amino)-5-methyl-3-oxohexanoate (5.0 g, 15.85 mmol, 1.0 eq) in DCM (10 mL) was added HCl/dioxane (5 mL) and the reaction mixture was stirred at RT for 1 h. After the reaction was completed, the reaction mixture was concentrated to give tert-butyl 5-amino-5-methyl-3-oxohexanoate (3.4 g, 99.9%). LC/MS (ESI): 216.2 [M+H]⁺.

Step 3: To a solution of tert-butyl 5-amino-5-methyl-3-oxohexanoate (3.1 g, 16.84 mmol, 1.0 eq) in DCM (18 mL) was added K₂CO₃ (4.98 g, 36.0 mmol, 2.5 eq) and cyclobutanone (5.43 mL, 70.09 mmol, 5.0 eq) and the reaction mixture was stirred at RT for 72 h. LC/MS showed desired mass. The reaction mixture was filtered and the filtrate was washed with water and brine, dried over Na₂SO₄ and concentrated. The crude product purified by silica gel chromatography eluted with PE: EA (3:1) to give tert-butyl 6,6-dimethyl-8-oxo-5-azaspiro[3.5]nonane-9-carboxylate (1.2 g, 31.1%). LC/MS (ESI): 268.2 [M+H]⁺.

Step 4: To a solution of tert-butyl 6,6-dimethyl-8-oxo-5-azaspiro[3.5]nonane-9-carboxylate (1200 mg, 4.49 mmol, 1.0 eq) in a mixture of H₂O (18 mL) and H₂SO₄ (2 mL) was added and the reaction mixture was stirred at 120° C. for 1 h. LC/MS showed desired mass. The reaction mixture was poured into ice water and the pH was adjusted to over 9. The aqueous layer was extracted with EA (3×20 mL). The organic layer was dried over Na₂SO₄ and concentrated under reduced pressure. The crude product was purified by silica gel chromatography eluted with DCM:MeOH (10:1) to give 6,6-dimethyl-5-azaspiro[3.5]nonan-8-one (620 mg, 81.27%). LC/MS (ESI): 168.2 [M+H]⁺.

Step 5: To a solution of 6,6-dimethyl-5-azaspiro[3.5]nonan-8-one (620 mg, 3.71 mmol, 1.0 eq) in MeOH (10 mL) was added NaBH₄ (140 mg, 3.71 mmol, 1.0 eq) at 0° C. The reaction mixture was stirred at RT for 1 h. LC/MS showed the desired mass. The reaction mixture was the diluted with H₂O and the pH was adjusted to around 7. The aqueous layer was extracted with EA (2×10 mL) and (DCM/propan-2-ol:IPA, 10:1). The organic layer was dried over Na₂SO₄ and concentrated under reduced pressure. The crude product was purified by silica gel chromatography eluted with DCM:MeOH (10:1, 0.5% TEA) to give 6,6-dimethyl-5-azaspiro[3.5]nonan-8-ol (540 mg, 86.01%). LC/MS (ESI): 170.2 [M+H]⁺.

Step 6: To a solution of 6,6-dimethyl-5-azaspiro[3.5]nonan-8-ol (540.0 mg, 3.19 mmol, 1.0 eq) in THF (10 mL) was added NaH (153.13 mg, 3.83 mmol, 1.2 eq, 60%) at 0° C. under N₂ atmosphere and the reaction mixture was stirred for 20 min. Then, 3,6-dibromo-1,2-diazine (758.88 mg, 3.19 mmol, 1.0 eq) was added and the resulting mixture was stirred at 60° C. overnight. Next the reaction mixture was cooled to RT and the mixture was poured into water. The aqueous layer was extracted with EA (3×10 mL). The organic layer was washed with brine, dried over Na₂SO₄ and evaporated to dryness. The crude product was purified by silica gel chromatography eluted with DCM:MeOH (10:1, 0.5% TEA) to give 8-((6-bromopyridazin-3-yl)oxy)-6,6-dimethyl-5-azaspiro[3.5]nonane (860 mg, 82.60%). LC/MS (ESI): 326.2 [M+H]⁺.

Step 7: 3-Bromo-6,7-dihydro-5H-pyrrolo[3,4-b]pyridin-5-one (211.0 mg, 1.0 mmol, 1.0 eq), 1,3-dimethyl-4-(4,4,5,5-tetramethyl-1,3,2-dioxaborolan-2-yl)-1H-pyrazole (244.1 mg, 1.10 mmol, 1.1 eq), aqueous potassium carbonate (2 M, 8 mL) and PdCl₂(dppf)·CH₂Cl₂ adduct (73.3 mg, 0.1 mmol, 0.1 eq) were charged to a three-neck 100 mL round bottom flask under nitrogen. Anhydrous dioxane (9 mL) was added and the mixture was heated to 85° C. overnight. Then the mixture was cooled, filtered through a pad of Celite, and the filter cake was washed with EA (100 mL). The filtrate was washed with brine and the organic layer was collected, dried over sodium sulfate, and concentrated in vacuo. The crude product was purified by silica gel column chromatography with DCM/MeOH 20/1, v/v to obtain 3-(1,3-dimethyl-1H-pyrazol-4-yl)-6,7-dihydro-5H-pyrrolo[3,4-b]pyridin-5-one (150.1 mg, 68%) as a brown solid. LC/MS (ESI): 229.1 [M+H]⁺.

Step 8: To a round bottom flask charged with 3-(1,3-dimethyl-1H-pyrazol-4-yl)-6,7-dihydro-5H-pyrrolo[3,4-b]pyridin-5-one (34.2 mg, 0.15 mmol, 1.0 eq), 8-((6-bromopyridazin-3-yl)oxy)-6,6-dimethyl-5-azaspiro[3.5]nonane (48.9 mg, 0.15 mmol, 1.1 eq), Cs₂CO₃ (148.9 mg, 0.46 mmol, 3.0 eq), and XantPhos Pd G4 (14.2 mg, 0.015 mmol, 0.1 eq) was added 1,4-dioxane (4 mL). The reaction mixture was degassed with argon for 5 min and was then heated to 90° C. for 8 h. The reaction mixture was cooled to RT and diluted with A. The organic phase was washed with brine, dried over sodium sulfate, and concentrated under vacuum. The crude product was purified by silica gel column chromatography with DCM/MeOH 20/1, v/v to obtain 5-(1-(tetrahydro-2H-pyran-2-yl)-1H-pyrazol-4-yl)isoindolin-1-one (42.2 mg), which was dissolved in HCl-dioxane (2 mL, 4.0 mol/L). The reaction was stirred at RT for 1 h, and was then concentrated. The resulting residue was purified by RP HPLC using MeCN and H₂O as eluent to provide 3-(1,3-dimethyl-1H-pyrazol-4-yl)-6-(6-((6,6-dimethyl-5-azaspiro[3.5]nonan-8-yl)oxy)pyridazin-3-yl)-6,7-dihydro-5H-pyrrolo[3,4-b]pyridin-5-one (4.94 mg, 6.9%) as a white solid.

¹H NMR (DMSO-d₆, 400 MHz) δ=8.95 (d, J=2.0 Hz, 1H), 8.71 (d, J=9.6 Hz, 1H), 8.22 (s, 1H), 8.19-8.15 (m, 2H), 7.35 (d, J=9.6 Hz, 1H), 5.57-5.46 (m, 1H), 5.20 (s, 2H), 3.82 (s, 3H), 2.57-2.52 (m, 1H), 2.36 (s, 3H), 2.21-2.07 (m, 4H), 1.94-1.85 (m, 1H), 1.83-1.65 (m, 2H), 1.55-1.40 (m, 2H), 1.21 (s, 3H), 1.16 (s, 3H); LC/MS (ESI): 474.2 [M+H]⁺.

### Example 20. Synthesis of 3-(2,4-dimethylthiazol-5-yl)-6-(6-(2-oxopiperazin-1-yl)pyridazin-3-yl)-6,7-dihydro-5H-pyrrolo[3,4-b]pyridin-5-one (Compound No. 255)

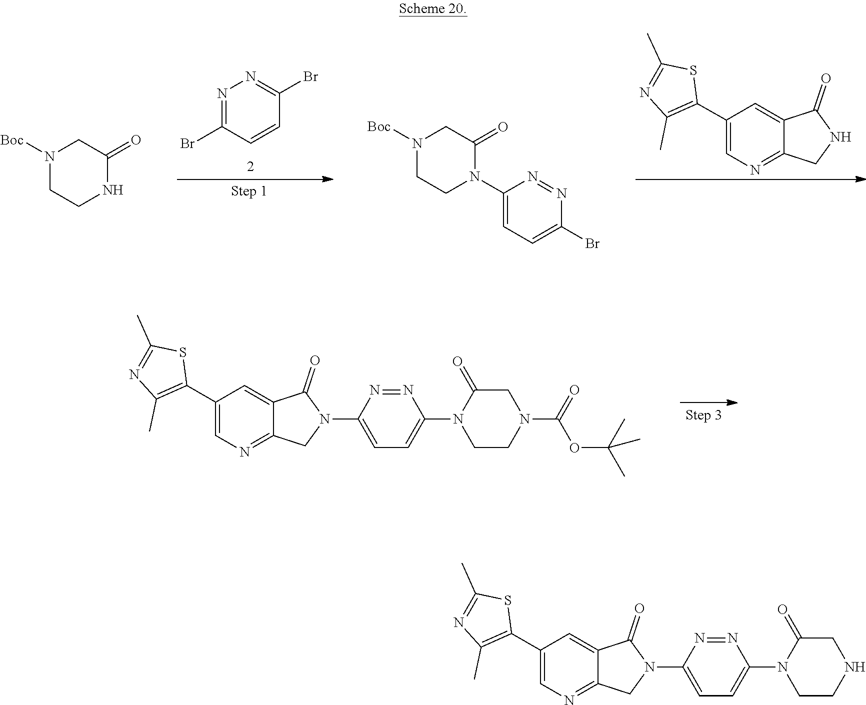 [SMILES: CC(C)(C)OC(=O)N1CCNC(=O)C1 ; Brc1ccc(Br)nn1 ; CC(C)(C)OC(=O)N1CCN(c2ccc(Br)nn2)C(=O)C1 ; Cc1nc(C)c(-c2cnc3c(c2)C(=O)NC3)s1 ; Cc1nc(C)c(-c2cnc3c(c2)C(=O)N(c2ccc(N4CCN(C(=O)OC(C)(C)C)CC4=O)nn2)C3)s1 ; Cc1nc(C)c(-c2cnc3c(c2)C(=O)N(c2ccc(N4CCNCC4=O)nn2)C3)s1]

Step 1: A solution of 2-methylpropan-2-yl 3-oxopiperazine-1-carboxylate (2.00 g, 9.99 mmol, 1.00 eq), 3,6-dibromo-1,2-diazine (11.89 g, 99.90 mmol, 5.00 eq), Cs₂CO₃ (9.76 g, 29.97 mmol, 3.00 eq), Pd(dba)Cl₂ (1.00 g, 1.00 mmol, 0.10 eq) and Xanphos (1.00 g, 2.00 mmol, 0.20 eq) in 1,4-dioxane (150 mL) was stirred at 100° C. for 16 h. After completion, the reaction mixture was concentrated under vacuum. The crude product was purified by silica gel column chromatography with PE/EA 20/1-5/1, v/v to provided 2-methylpropan-2-yl-4-(6-bromo-1,2-diazin-3-yl)-3-oxopiperazine-1-carboxylate (655 mg, 1.83 mmol, 18.36%) as a yellow solid. LC/MS (ESI): 357.2 [M+H]⁺.

Step 2: A mixture of 3-(2,4-dimethyl-1,3-thiazol-5-yl)-6,7-dihydro-5H-pyrrolo[4,3-b]pyridin-5-one (100 mg, 0.41 mmol, 1.0 eq), 2-methylpropan-2-yl 4-(6-bromo-1,2-diazin-3-yl)-3-oxopiperazine-1-carboxylate (174.75 mg, 0.49 mmol, 1.2 eq), Cs₂CO₃ (398.48 mg, 1.22 mmol, 3.0 eq) and XantPhos Pd G4 (66.04 mg, 0.04 mmol, 0.1 eq) in dioxane (1 mL) was stirred under N₂ at 100° C. for 3 h. The reaction mixture was cooled to RT and concentrated under vacuum. The crude product was purified by silica gel column chromatography with DCM/MeOH 20/1, v/v to provided 2-methylpropan-2-yl 4-{6-[3-(2,4-dimethyl-1,3-thiazol-5-yl)-5-oxo-6,7-dihydro-5H-pyrrolo[4,3-b]pyridin-6-yl]-1,2-diazin-3-yl}-3-oxopiperazine-1-carboxylate (100 mg, 47.03%) as a brown solid. LC/MS (ESI): 522.18 [M+H]⁺.

Step 3: A solution of 2-methylpropan-2-yl 4-{6-[3-(2,4-dimethyl-1,3-thiazol-5-yl)-5-oxo-6,7-dihydro-5H-pyrrolo[4,3-b]pyridin-6-yl]-1,2-diazin-3-yl}-3-oxopiperazine-1-carboxylate (100 mg, 0.19 mmol) in HCl (g) in 1,4-dioxane (4 M, 4 mL) was stirred at RT for 1 h. The reaction mixture was concentrated under vacuum. The crude product was purified by column chromatography (SiO₂, DCM:MeOH=50:1-25:1-10:1). The product was triturated with MeOH and filtered to provided 3-(2,4-dimethyl-1,3-thiazol-5-yl)-6-[6-(2-oxopiperazin-1-yl)-1,2-diazin-3-yl]-6,7-dihydro-5H-pyrrolo[4,3-b]pyridin-5-one (20.28 mg, 25.09%) as a white solid. LC/MS (ESI): 422.1 [M+H]⁺. ¹H NMR (400 MHz, DMSO-d₆) δ=9.77 (br s, 2H), 8.98 (d, J=2.1 Hz, 1H), 8.84 (d, J=9.6 Hz, 1H), 8.27 (d, J=2.0 Hz, 1H), 8.20 (d, J=9.8 Hz, 1H), 5.29 (s, 2H), 4.31-4.22 (m, 2H), 4.02 (s, 2H), 3.65-3.58 (m, 2H), 2.68 (s, 3H), 2.43 (s, 3H); LC/MS (ESI): 422.95 [M+H]⁺.

### Example 21. Synthesis of (S)-6-(6-(3-isopropylpiperazin-1-yl)pyridazin-3-yl)-3-(1-methyl-1H-pyrazol-4-yl)-6,7-dihydro-5H-pyrrolo[3,4-b]pyridin-5-one (Compound No. 157)

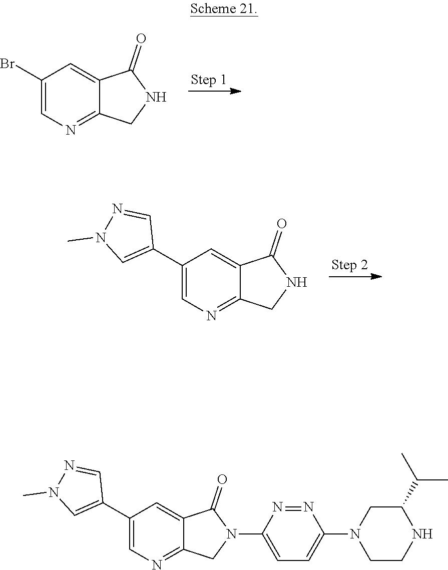 [SMILES: O=C1NCc2ncc(Br)cc21 ; Cn1cc(-c2cnc3c(c2)C(=O)NC3)cn1 ; CC(C)[C@H]1CN(c2ccc(N3Cc4ncc(-c5cnn(C)c5)cc4C3=O)nn2)CCN1]

Step 1: 3-Bromo-6,7-dihydro-5H-pyrrolo[3,4-b]pyridin-5-one (213.0 mg, 1.0 mmol, 1.0 eq), 1-methyl-4-(4,4,5,5-tetramethyl-1,3,2-dioxaborolan-2-yl)-1H-pyrazole (312. mg, 1.5 mmol, 1.5 eq), aqueous potassium carbonate (2 M, 2 mL) and PdCl₂(dppf)·CH₂Cl₂ adduct (73.2 mg, 0.1 mmol, 0.1 eq) were charged to a three-neck 100 mL round bottom flask under nitrogen. Anhydrous dioxane (9 mL) was added and the mixture was heated to 85° C. overnight. Then the mixture was cooled, filtered through a pad of Celite, and the filter cake with rinsed with EA (100 mL). The filtrate was washed with brine and the organic layer was collected, dried over sodium sulfate, and concentrated in vacuo. The crude product was purified by silica gel column chromatography with DCM/MeOH 20/1, v/v to obtain 3-(1-methyl-1H-pyrazol-4-yl)-6,7-dihydro-5H-pyrrolo[3,4-b]pyridin-5-one (183.2 mg, 85.2%) as a brown solid. LC/MS (ESI): 215.1 [M+H]⁺.

Step 2: To a round bottom flask charged with 3-(1-methyl-1H-pyrazol-4-yl)-6,7-dihydro-5H-pyrrolo[3,4-b]pyridin-5-one (32.2 mg, 0.15 mmol, 1.0 eq), tert-butyl (S)-4-(6-bromopyridazin-3-yl)-2-isopropylpiperazine-1-carboxylate (57.6 mg, 0.15 mmol, 1.1 eq), Cs₂CO₃ (148.9 mg, 0.46 mmol, 3.0 eq), and XantPhos Pd G4 (14.2 mg, 0.015 mmol, 0.1 eq) was added 1,4-dioxane (4 mL). The reaction mixture was degassed with argon for 5 min and was then heated at 90° C. for 8 h. The reaction mixture was cooled to RT and was diluted with EA. The organic phase was washed with brine, dried over sodium sulfate, and was concentrated under vacuum. The crude product was purified by silica gel column chromatography with DCM/MeOH 20/1, v/v, and was then dissolved HCl in dioxane (2 mL, 4.0 mol/L). The reaction was stirred at RT for 1 h, and was then concentrated. The resulting residue was purified by RP HPLC using MeCN and H₂O as eluent to provide 3-(1-ethyl-3-(trifluoromethyl)-1H-pyrazol-4-yl)-6-(6-((2,2,6,6-tetramethylpiperidin-4-yl)oxy)pyridazin-3-yl)-6,7-dihydro-5H-pyrrolo[3,4-b]pyridin-5-one (9.51 mg, 15.2%) as a white solid.

¹H NMR (DMSO-d₆, 400 MHz) δ 9.12 (d, 1H, J=2.0 Hz), 8.48 (d, 1H, J=10.0 Hz), 8.44 (s, 1H), 8.37 (d, 1H, J=2.1 Hz), 8.18 (s, 1H), 8.14 (s, 1H), 7.48 (d, 1H, J=9.9 Hz), 5.14 (s, 2H), 4.27-4.34 (m, 1H), 4.11-4.19 (m, 1H), 3.90 (s, 3H), 3.02-3.15 (m, 1H), 2.82-2.92 (m, 1H), 2.79-2.86 (m, 1H), 2.62-2.72 (m, 2H), 1.66-1.72 (m, 1H), 0.97 (d, 6H, J=6.8 Hz); LC/MS (ESI): 419.2 [M+H]⁺.

### Example 22. Synthesis of 3-(1,3-dimethyl-1H-pyrazol-4-yl)-6-(3-(((1R,3s,5S)-1,5-dimethyl-8-azabicyclo[3.2.1]octan-3-yl)(methyl)amino)-1,2,4-triazin-6-yl)-6,7-dihydro-5H-pyrrolo[3,4-b]pyridin-5-one (Compound No. 175)

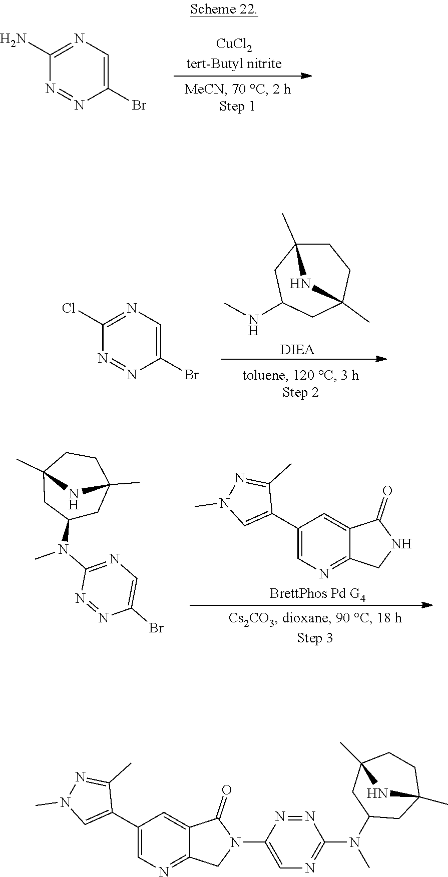

Step 1: To a solution of 6-bromo-1,2,4-triazin-3-amine (2.0 g, 11.43 mmol) and CuCl₂ (1.47 g, 14.86 mmol) in MeCN (25 mL) was added isoamyl nitrite (2.18 mL, 18.29 mmol) and the reaction mixture was stirred at 70° C. for 2 h. The reaction mixture was then concentrated and the resulting residue was purified by column chromatography (SiO₂, PE:EA=10:1) to give 6-bromo-3-chloro-1,2,4-triazine (220 mg, 1.13 mmol, 9.90%) as a yellow solid.

Step 2: To a solution of 6-bromo-3-chloro-1,2,4-triazine (200 mg, 1.03 mmol) and (1R,5S)—N, 1,5-trimethyl-8-azabicyclo[3.2.1]octan-3-amine (192.71 mg, 1.13 mmol) in toluene (10 mL) was added DIEA (0.34 mL, 2.06 mmol) and the reaction mixture was stirred at 120° C. for 3 h. LC/MS showed the reaction was completed. The reaction mixture was concentrated and the resulting residue was purified by column chromatography (SiO₂, DCM:MeOH=10:1) to give (1R,3s,5S)—N-(6-bromo-1,2,4-triazin-3-yl)-N, 1,5-trimethyl-8-azabicyclo[3.2.1]octan-3-amine (0.23 g, 67.65%) as a yellow solid.

Step 3: To a solution of 3-(1,3-dimethylpyrazol-4-yl)-6,7-dihydro-5H-pyrrolo[4,3-b]pyridin-5-one (35 mg, 0.15 mmol) and (1R,3S,5S)—N-(6-bromo-1,2,4-triazin-3-yl)-N, 1,5-trimethyl-8-azabicyclo[3.2.1]octan-3-amine (52 mg, 0.16 mmol) in dioxane (10 mL) was added BrettPhos Pd G4 (13.61 mg, 0.01 mmol) and Cs₂CO₃ (146.62 mg, 0.45 mmol); and the reaction mixture was stirred at 90° C. for 16 h under N₂. LC/MS showed the reaction was completed. The reaction mixture was filtered and concentrated. The resulting residue was purified by pre-HPLC (TFA) to give 3-(1,3-dimethyl-1H-pyrazol-4-yl)-6-(3-(((1R,5S)-1,5-dimethyl-8-azabicyclo[3.2.1]octan-3-yl)(methyl)amino)-1,2,4-triazin-6-yl)-6,7-dihydro-5H-pyrrolo[3,4-b]pyridin-5-one (30.25 mg, 39.86%) as a yellow solid.

¹H NMR (DMSO-d₆, 400 MHz) δ=9.34 (s, 1H), 8.94 (d, J=1.6 Hz, 1H), 8.18 (s, 2H), 5.13 (s, 2H), 5.01-5.10 (m, 1H), 3.82 (s, 3H), 3.02 (s, 3H), 2.36 (s, 3H), 1.78 (br d, J=7.4 Hz, 2H), 1.51-1.65 (m, 6H), 1.20 (s, 6H), 1H not observed (NH); LC/MS (ESI): 474.3 [M+H]⁺.

### Example 23. Synthesis of 6-(6-((7-azadispiro[5.1.58.36]hexadecan-15-yl)oxy)pyridazin-3-yl)-3-(1,3-dimethyl-1H-pyrazol-4-yl)-6,7-dihydro-5H-pyrrolo[3,4-b]pyridin-5-one (Compound No. 243)

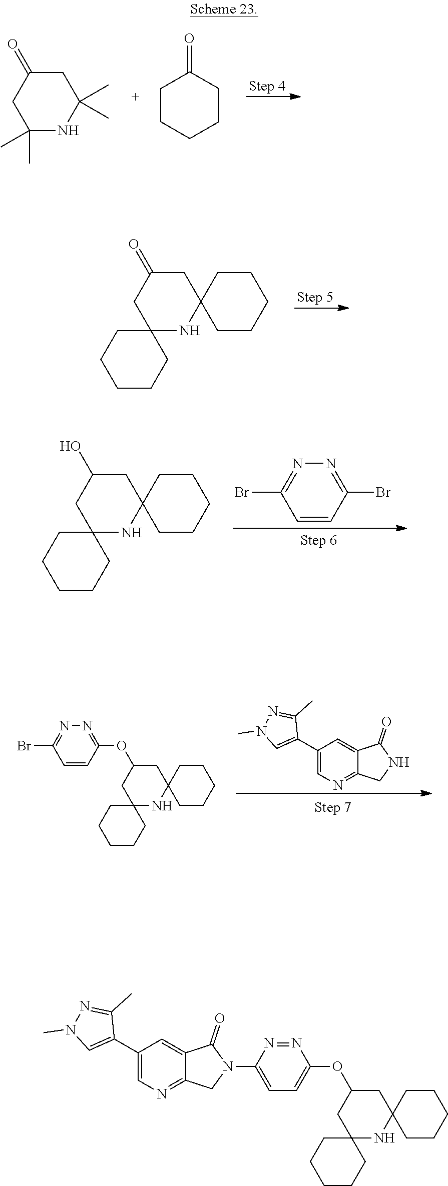

Step 1: A solution of ethyl 5-bromo-2-methylnicotinate (3.00 g, 12.35 mmol, 1.00 eq), benzoyl peroxide (5.98 g, 24.70 mmol, 2.00 eq) and N-bromosuccinimide (4.37 g, 24.70 mmol, 2.00 eq) in carbon tetrachloride (30 mL) was stirred at 80° C. for 16 h. After consumption of the starting material, the reaction mixture was concentrated under vacuum. The crude product was purified by silica gel column chromatography with PE/EA 20/1~5/1, v/v to provided ethyl 5-bromo-2-(bromomethyl)nicotinate (2.37 g, 7.41 mmol, 61.03%) as a white solid. LC/MS (ESI): 324.0 [M+H]⁺.

Step 2: A solution of ethyl 5-bromo-2-(bromomethyl)nicotinate (2.00 g, 6.23 mmol, 1.00 eq) and ammonia (1.06, 62.30 mmol, 10.0 eq) in isopropanol (10 mL) was stirred at 25° C. for 6 h. After consumption of the starting material, the reaction mixture was concentrated under vacuum. The crude product was purified by silica gel column chromatography with DCM/MeOH 50/1-10/1, v/v to provided 3-bromo-6,7-dihydro-5H-pyrrolo[3,4-b]pyridin-5-one (1.06 g, 5.04 mmol, 81.32%) as a white solid. LC/MS (ESI): 214.0 [M+H]⁺.

Step 3: 3-Bromo-6,7-dihydro-5H-pyrrolo[3,4-b]pyridin-5-one (1.00 g, 5.0 mmol, 1.00 eq), 2,4-dimethyl-5-(4,4,5,5-tetramethyl-1,3,2-dioxaborolan-2-yl)thiazole) (1.31 g, 5.50 mmol, 1.10 eq), aqueous potassium carbonate (2 M, 8 mL) and PdCl₂(dppf)·CH₂Cl₂ adduct (365.7 mg, 0.50 mmol, 0.1 eq) were charged to a three-neck 100 mL round bottom flask under nitrogen. Anhydrous dioxane (9 mL) was added and the mixture was heated to 85° C. overnight. Then the mixture was cooled, filtered through a pad of Celite, and the filter cake was rinsed with EA (100 mL). The filtrate was washed with brine and the organic layer was collected, dried over sodium sulfate, and concentrated in vacuo. The crude product was purified by silica gel column chromatography with DCM/MeOH 20/1, v/v to obtain 3-(2,4-dimethylthiazol-5-yl)-6,7-dihydro-5H-pyrrolo[3,4-b]pyridin-5-one (335 mg, 68%) as a brown solid.

LC/MS (ESI): 246.0 [M+H]⁺.

Step 4: A solution of 2,2,6,6-tetramethylhexahydropyridin-4-one (10.0 g, 64.42 mmol, 1.00 eq), cyclohexanone (18.97 g, 193.25 mmol, 3.00 eq) and NH₄Cl (20.67 g, 386.50 mmol, 6.00 eq) in DMSO (30 mL) was stirred at 80° C. for 48 h. After consumption of the starting material, the mixture was cooled to room temperature and diluted with 100 mL of water. The aqueous layer was extracted with EA (3×100 mL). The organic layer was washed with brine (100 mL), dried over sodium sulfate, filtered, and concentrated. The crude product was purified by silica gel column chromatography with PE/EA 20/1~1/1, v/v to provided spiro[1-azaspiro[5.5]undecane-2,1′-cyclohexane]-4-one (15 g, 57.99 mmol, 90.03%) as a white solid. LC/MS (ESI): 237.2 [M+H]⁺.

Step 5: To a solution of spiro[1-azaspiro[5.5]undecane-2,1′-cyclohexane]-4-one (6.40 g, 27.19 mmol, 1.00 eq) in MeOH (65 mL), NaBH₄ (0.51 g, 13.60 mmol, 0.50 eq) was added at 0° C.; and the reaction mixture was stirred at room temperature for 3 h. After consumption of the starting material, the reaction mixture was concentrated under vacuum. The crude product was purified by silica gel column chromatography with DCM/MeOH 50/1-20/1, v/v to provided spiro[1-azaspiro[5.5]undecane-2,1′-cyclohexane]-4-ol (3.40 g, 14.32 mmol, 52.67%) as a yellow oil. LC/MS (ESI): 239.2 [M+H]⁺.

Step 6: To a solution of spiro[1-azaspiro[5.5]undecane-2,1′-cyclohexane]-4-ol (3.00 g, 12.64 mmol, 1.00 eq) in THF (30 mL), NaH (0.76 g, 31.59 mmol, 2.50 eq) was added dropwise over 30 min at 0° C. Next the resulting mixture was stirred under N₂ at 25° C. for 0.5 h. Then 3,6-dibromo-1,2-diazine (3.61 g, 15.16 mmol, 1.20 eq) was added and the reaction mixture was stirred under N₂ at 25° C. for 24 h.

After consumption of the starting material, the reaction mixture was quenched with saturated ammonium chloride and the reaction was concentrated under vacuum. The residue was extracted with EA (3×30 mL) and the organic layer was washed with brine, dried over sodium sulfate, and concentrated in vacuo.

The crude product was purified by silica gel column chromatography with PE/EtOAc 20/1~1/1 to provided 4-[(6-bromo-1,2-diazin-3-yl)oxy]spiro[1-azaspiro[5.5]undecane-2,1′-cyclohexane](1.80 g, 4.43 mmol, 35.03%) as a yellow solid. LC/MS (ESI): 395.3 [M+H]⁺.

Step 7: To a solution of4-[(6-bromo-1,2-diazin-3-yl)oxy]spiro[1-azaspiro[5.5]undecane-2,1′-cyclohexane](0.9 g, 2.28 mmol, 1.00 eq), 3-(1,3-dimethylpyrazol-4-yl)-6,7-dihydro-5H-pyrrolo[4,3-b]pyridin-5-one (0.63 g, 2.74 mmol, 1.20 eq) and Cs₂CO₃ (2.23 g, 6.85 mmol, 3.00 eq) in 1,4-dioxane (15 mL) was added XPhos Pd G4 (0.20 g, 0.23 mmol, 0.10 eq) at 25° C.; and the reaction mixture was stirred under N₂ at 90° C. for 4 h. After consumption of the starting material, the reaction mixture was concentrated under vacuum. The crude product was purified by silica gel column chromatography with DCM/MeOH 50/1~10/1, v/v to provided 3-(1,3-dimethylpyrazol-4-yl)-6-[6-(spiro[l1-azaspiro[5.5]undecane-2,1′-cyclohexane]-4-yloxy)-1,2-diazin-3-yl]-6,7-dihydro-5H-pyrrolo[4,3-b]pyridin-5-one (0.43 g, 0.79 mmol, 34.82%) as a white solid.

¹H NMR (400 MHz, DMSO-d₆) δ=8.95 (d, J=2.0 Hz, 1H), 8.70 (d, J=9.6 Hz, 1H), 8.21-8.16 (m, 3H), 7.32 (d, J=9.6 Hz, 1H), 5.61-5.51 (m, 1H), 5.20 (s, 2H), 3.82 (s, 3H), 2.36 (s, 3H), 1.79-1.49 (m, 10H), 1.49-1.30 (m, 14H); LC/MS (ESI): 542.4 [M+H]⁺.

### Example 24. Synthesis of 6-(2,5-dimethyl-2H-1,2,3-triazol-4-yl)-2-(6-(((1R,3s,5S)-1,5-dimethyl-8-azabicyclo[3.2.1]octan-3-yl)oxy)pyridazin-3-yl)-4-fluoroisoindolin-1-one (Compound No. 283)

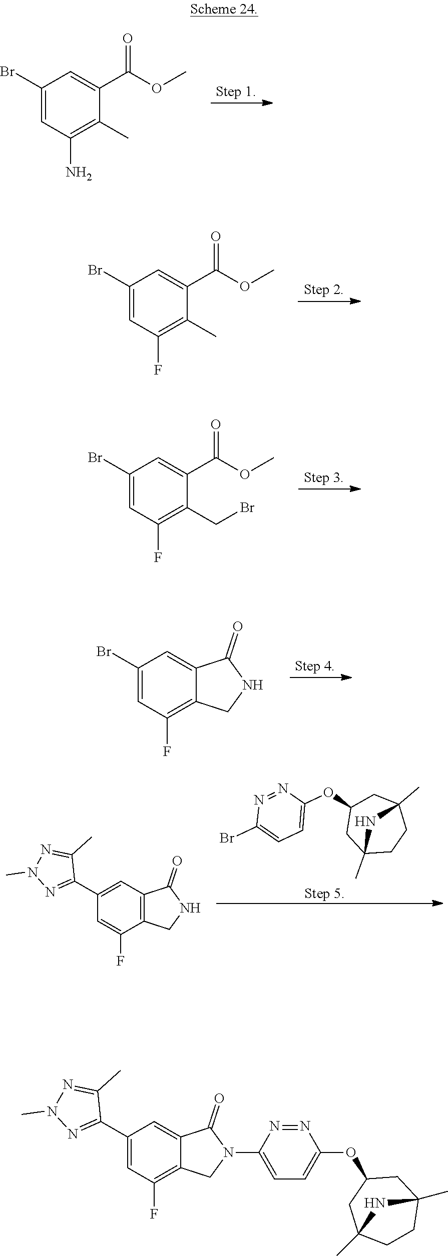

Step 1: To the solution of methyl 3-amino-5-bromo-2-methylbenzoate (1.0 g, 4.10 mmol) in DCM (10 mL), nitrosonium tetrafluoroborate, (480 mg, 4.10 mmol) was added portionwise at 0° C. under N₂; and the reaction mixture was stirred at 0° C. for 1 h. Then oxylene (30 mL) was added and the reaction mixture was refluxed at 130° C. for 3 h. The reaction mixture was cooled to room temperature and poured into H₂O and the aqueous layer was extracted EA. The organic layer was washed with brine, dried over MgSO₄, filtered, and concentrated. The crude product was purified by silica gel column chromatography with PE/EA 10/1, v/v to afford methyl 5-bromo-3-fluoro-2-methylbenzoate (860 mg, 85%).

Step 2: To the solution of 5-bromo-3-fluoro-2-methylbenzoate (860 mg, 3.5 mmol) and NBS (623 mg, 3.5 mmol) in (trifluoromethyl)benzene (10 mL), was added AIBN (57 mg, 0.35 mmol); and the reaction mixture was stirred under N₂ at 100° C. for 16 h. Then the reaction mixture was cooled to room temperature and was poured into H₂O. The aqueous layer was extracted EA. The organic layer was washed with brine, dried over MgSO₄, filtered, and concentrated. The crude product was purified by silica gel column chromatography with PE/EA 10/1, v/v to afford methyl 5-bromo-2-(bromomethyl)-3-fluorobenzoate (800 mg, 70%).

Step 3: To a solution of methyl 5-bromo-2-(bromomethyl)-3-fluorobenzoate (800 mg, 2.5 mmol) in THF (7 mL), NH₄OH (7 mL) was added and the reaction mixture was stirred at RT for 4 h. The reaction mixture was filtered and concentrated under vacuum. The crude product was used for directly in the next step.

Step 4: 6-Bromo-4-fluoroisoindolin-1-one (343.0 mg, 1.5 mmol, 1.5 eq), 4,4,4′,4′,5,5,5′,5′-octamethyl-2,2′-bi(1,3,2-dioxaborolane (762 mg, 3.0 mmol, 3.0 eq), potassium acetate (300 mg, 3.0 mmol, 3.0 eq) and PdCl₂(dppf)·CH₂Cl₂ adduct (109.3 mg, 0.15 mmol, 0.1 eq) were charged to a three-neck 100 mL round bottom flask under nitrogen. Anhydrous dioxane (9 mL) was added and the mixture was heated to 90° C. When full conversion was achieved, the reaction mixture was cooled to room temperature. Then 3-bromo-6,7-dihydro-5H-pyrrolo[3,4-b]pyridin-5-one (211.9 mg, 1.0 mmol, 1.0 eq), aqueous potassium carbonate (2 M, 2 mL) and PdCl₂(dppf)·CH₂Cl₂ adduct (109.3 mg, 0.15 mmol, 0.1 eq) were added and the mixture was heated to 90° C. When full conversion was achieved, the mixture was cooled, filtered through a pad of Celite, and the filter cake was rinsed with EA (100 mL). The filtrate was washed with brine and the organic layer was collected, dried over sodium sulfate, and concentrated in vacuo. The crude product was purified by silica gel column chromatography with DCM/MeOH 20/1, v/v to obtain 6-(2,5-dimethyl-2H-1,2,3-triazol-4-yl)-4-fluoroisoindolin-1-one (81.5 mg, 33%) as a brown solid. LC/MS (ESI): 247.1 [M+H]⁺.

Step 5: To a round bottom flask charged with 6-(2,5-dimethyl-2H-1,2,3-triazol-4-yl)-4-fluoroisoindolin-1-one (36.9 mg, 0.15 mmol, 1.0 eq), tert-butyl (1R,3S,5S)-3-((6-bromopyridazin-3-yl)oxy)-1,5-dimethyl-8-azabicyclo[3.2.1]octane-8-carboxylate (61.8 mg, 0.15 mmol, 1.1 eq), Cs₂CO₃ (148.9 mg, 0.46 mmol, 3.0 eq), and XantPhos Pd G4 (14.2 mg, 0.015 mmol, 0.1 eq) was added 1,4-dioxane (4 mL). The reaction mixture was degassed with argon for 5 min and was then heated at 90° C. for 8 h. The reaction mixture was cooled to RT and was then diluted with EA. The organic phase was washed with brine, dried over sodium sulfate, and concentrated under vacuum. The crude product was purified by silica gel column chromatography with DCM/MeOH 20/1, v/v, and was then dissolved HCl in dioxane (2 mL, 4.0 mol/L). The reaction was stirred at RT for 1 h, and was then concentrated. The resulting residue was purified by RP HPLC using MeCN and H₂O as eluent to provide 6-(1,3-dimethyl-1H-pyrazol-4-yl)-2-(6-(((1R,3s,5S)-1,5-dimethyl-8-azabicyclo[3.2.1]octan-3-yl)oxy)pyridazin-3-yl)-4-fluoroisoindolin-1-one (28.8 mg, 40.3%) as a white solid.

¹H NMR (400 MHz, DMSO-d₆) δ: 9.42 (br d, J=7.4 Hz, 1H), 9.31-9.17 (m, 1H), 8.72 (d, J=9.6 Hz, 1H), 7.89 (s, 1H), 7.83 (dd, J=0.8, 9.9 Hz, 1H), 7.39 (d, J=9.6 Hz, 1H), 5.52 (td, J=5.3, 10.8 Hz, 1H), 5.27 (s, 2H), 4.16 (s, 3H), 2.48 (s, 3H), 2.41 (br dd, J=5.8, 13.3 Hz, 2H), 2.13 (br d, J=8.0 Hz, 2H), 1.95-1.81 (m, 4H), 1.48 (s, 6H); LC/MS (ESI): 478.2 [M+H]⁺.

### Example 25. Synthesis of (S)-2-(6-(3-(tert-butyl)piperazin-1-yl)pyridazin-3-yl)-6-(1,3-dimethyl-1H-pyrazol-4-yl)-4-fluoroisoindolin-1-one (Compound No. 295)

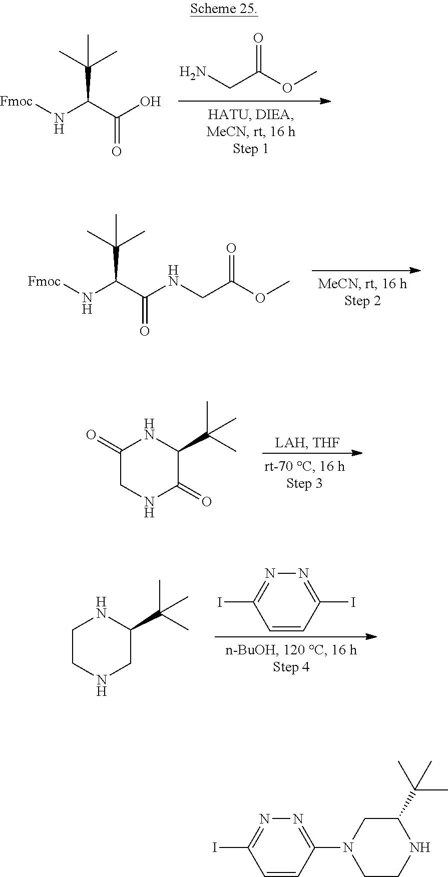

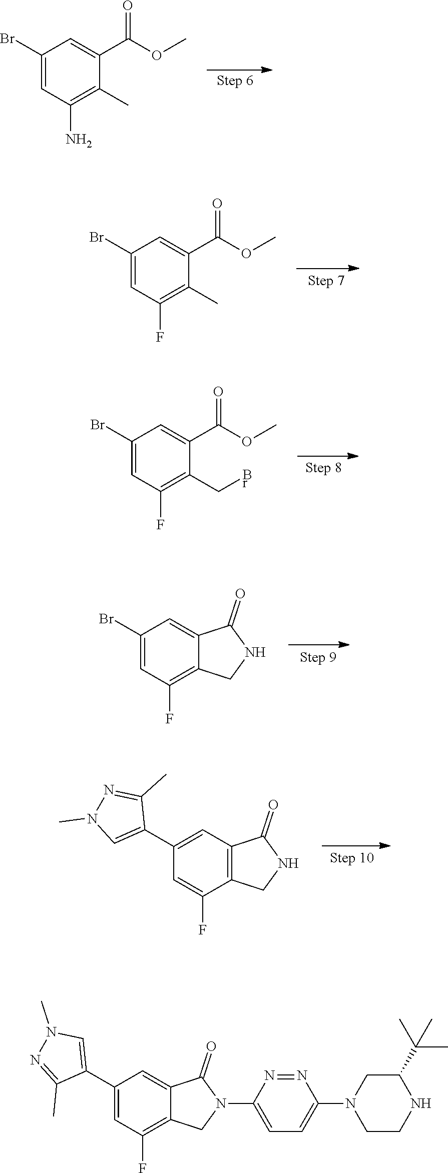 [SMILES: COC(=O)c1cc(Br)cc(N)c1C ; COC(=O)c1cc(Br)cc(F)c1C ; COC(=O)c1cc(Br)cc(F)c1CBr ; O=C1NCc2c(F)cc(Br)cc21 ; Cc1nn(C)cc1-c1cc(F)c2c(c1)C(=O)NC2 ; Cc1nn(C)cc1-c1cc(F)c2c(c1)C(=O)N(c1ccc(N3CCN[C@@H](C(C)(C)C)C3)nn1)C2]

Step 1: To a solution of (2S)-2-({[(9H-fluoren-9-ylmethyl)oxy]carbonyl}amino)-3,3-dimethylbutanoic acid (10 g, 28.29 mmol, 1.0 eq) and methyl aminoacetate (3.0 g, 33.95 mmol, 1.2 eq) in acetonitrile (50 mL) was added DIEA (9.1 g, 70.74 mmol, 2.5 eq) and HATU (11.8 g, 31.12 mmol, 1.1 eq), and the reaction mixture was stirred at RT for 16 h. Next the reaction mixture was filtered to provided 9H-fluoren-9-ylmethyl {[(7S)-8,8-dimethyl-3,6-dioxo-5-aza-2-oxanon-7-yl]amino}methanoate (9.5 g, 22.38 mmol, 79.09%) as a sightly yellow solid. LC/MS (ESI): 425.5 [M+H]⁺.

Step 2: A solution of 9H-fluoren-9-ylmethyl{[(7S)-8,8-dimethyl-3,6-dioxo-5-aza-2-oxanon-7-yl]amino}methanoate (5.0 g, 11.78 mmol, 1.0 eq) in acetonitrile (100 mL) was stirred RT for 16 h. After the reaction was completed, the reaction mixture was concentrated in vacuo to provided 9H-fluoren-9-ylmethyl {[(7S)-8,8-dimethyl-3,6-dioxo-5-aza-2-oxanon-7-yl]amino}methanoate (4.85 g, 11.43 mmol, 97.00%) as a sightly yellow solid. LC/MS (ESI): 171.2 [M+H]⁺.

Step 3: LiAlH₄ (2.14 g, 56.40 mmol, 6.0 eq) was added to a vigorous stirred solution of (3S)-3-(2-methylprop-2-yl)piperazine-2,5-dione (1.6 g, 9.40 mmol, 1.0 eq) in THF (20 mL) in an ice bath, and the resulting reaction mixture was stirred at 70° C. for 16 h. Next the mixture was poured into KOH (6N), and the mixture was stirred in an ice bath for 1 h and was then filtered. The filtrate was extracted with EA, washed with brine, dried over Na₂SO₄, and concentrated in vacuo to provided (2S)-2-(2-methylprop-2-yl)piperazine (1.2 g, 8.44 mmol, 89.74%) as a yellow oil. LC/MS (ESI): 143.2 [M+H]⁺.

Step 4: DIEA (1.13 g, 8.79 mmol, 1.46 mL, 2.5 eq) was added to a vigorous stirred solution of (2S)-2-(2-methylprop-2-yl)piperazine (0.5 g, 3.51 mmol, 1.0 eq) and 3,6-diiodo-1,2-diazine (1.17 g, 3.51 mmol, 1.0 eq) in n-BuOH (7 mL) at RT, and the reaction mixture was stirred at 120° C. for 16 h. Next the mixture was concentrated in vacuo and the resulting residue was diluted with EA, washed with water and brine, dried over Na₂SO₄, and concentrated in vacuo to provided 6-iodo-3-[(3S)-3-(2-methylprop-2-yl)piperazin-1-yl]-1,2-diazine (450 mg, 1.30 mmol, 36.98%) as a yellow solid. LC/MS (ESI): 347.2 [M+H]⁺.

Step 5: Pd(dppf)Cl₂ (49 mg, 0.06 mmol, 0.05 eq) was added to a vigorous stirred solution of 1-methyl-4-(4,4,5,5-tetramethyl-1,3,2-dioxaborolan-2-yl)pyrazole (250 mg, 1.20 mmol, 1.0 eq), 3-bromo-6,7-dihydro-5H-pyrrolo[4,3-b]pyridin-5-one (256 mg, 1.20 mmol, 1.0 eq) and K₂CO₃ (332 mg, 2.40 mmol, 2.0 eq) in dioxane (5 mL) and water (1 mL) and the resulting reaction mixture was stirred at 80° C. for 16 h. The reaction mixture was filtered and the filtrate was concentrated in vacuo. The crude product was purified by silica gel column chromatography with DCM/MeOH 10/1, v/v to afford 3-(1-methylpyrazol-4-yl)-6,7-dihydro-5H-pyrrolo[4,3-b]pyridin-5-one (135 mg, 0.63 mmol, 52.51%) as a sightly yellow solid. LC/MS (ESI): 215.2 [M+H]⁺.

Step 6: To the solution of methyl 3-amino-5-bromo-2-methylbenzoate (1.0 g, 4.10 mmol) in DCM (10 mL), was nitrosonium tetrafluoroborate, (480 mg, 4.10 mmol) was added portionwise at 0° C. under N₂ and the resulting reaction mixture was stirred at 0° C. for 1 h. Then oxylene (30 mL) was added and the reaction mixture was refluxed at 130° C. for 3 h. The reaction mixture was cooled to room temperature and poured into H₂O. The aqueous layer was extracted EA. The organic layer was washed with brine, dried over MgSO₄, filtered, and concentrated. The crude product was purified by silica gel column chromatography with PE/EA 10/1, v/v to obtain methyl 5-bromo-3-fluoro-2-methylbenzoate (860 mg, 85%).

Step 7: To the solution of 5-bromo-3-fluoro-2-methylbenzoate (860 mg, 3.5 mmol) and NBS (623 mg, 3.5 mmol) in (trifluoromethyl)benzene (10 mL), was AIBN (57 mg, 0.35 mmol) was added; and the reaction mixture was stirred under N₂ at 100° C. for 16 h. The reaction mixture was then cooled to room temperature and poured into H₂O. The aqueous layer was extracted EA. The organic layer was washed with brine, dried over MgSO₄, filtered, and concentrated. The crude product was purified by silica gel column chromatography with PE/EA 10/1, v/v to obtain methyl 5-bromo-2-(bromomethyl)-3-fluorobenzoate (800 mg, 70%).

Step 8: To a solution of methyl 5-bromo-2-(bromomethyl)-3-fluorobenzoate (800 mg, 2.5 mmol) dissolved in THF (7 mL), NH₄OH (7 mL) was added and the reaction was stirred at RT for 4. The reaction mixture was filtered and concentrated under vacuum. The crude product was used directly in the next step.

Step 9: 6-Bromo-4-fluoroisoindolin-1-one (228 mg, 1.0 mmol, 1.0 eq), 1,3-dimethyl-4-(4,4,5,5-tetramethyl-1,3,2-dioxaborolan-2-yl)-1H-pyrazole (244.1 mg, 1.10 mmol, 1.1 eq), aqueous potassium carbonate (2 M, 8 mL) and PdCl₂(dppf)·CH₂Cl₂ adduct (73.3 mg, 0.1 mmol, 0.1 eq) were charged to a three-neck 100 mL round bottom flask under nitrogen. Anhydrous dioxane (9 mL) was added and the reaction mixture was heated to 85° C. overnight. Then the mixture was cooled, filtered through a pad of Celite, and the filter cake was rinsed with EA (100 mL). The filtrate was washed with brine and the organic layer was collected, dried over sodium sulfate, and concentrated in vacuo. The crude product was purified by silica gel column chromatography with DCM/MeOH 20/1, v/v to obtain 6-(1,3-dimethyl-1H-pyrazol-4-yl)-4-fluoroisoindolin-1-one (134.7 mg, 55%) as a brown solid. LC/MS (ESI): 246.1 [M+H]⁺.

Step 10: 6-(1,3-Dimethyl-1H-pyrazol-4-yl)-4-fluoroisoindolin-1-one (50 mg, 0.23 mmol, 1.0 eq) and 6-iodo-3-[(3S)-3-(2-methylprop-2-yl)piperazin-1-yl]-1,2-diazine (80 mg, 0.23 mmol, 1.0 eq) were dissolved in DMF (2 mL), and CuI (4 mg, 0.02 mmol, 0.10 eq), K₂CO₃ (97 mg, 0.70 mmol, 3.0 eq) and (1R,2R)-cyclohexane-1,2-diamine (2.7 mg, 0.02 mmol, 0.10 eq) were added and the resulting reaction mixture was stirred at 100° C. for 16 h. Next the reaction mixture was filtered and the filtrate was concentrated. The crude product was purified by pre-HPLC to provided (S)-2-(6-(3-(tert-butyl)piperazin-1-yl)pyridazin-3-yl)-6-(1,3-dimethyl-1H-pyrazol-4-yl)-4-fluoroisoindolin-1-one (24.19 mg, 21.55%) as a white solid.

¹H NMR (DMSO-d₆, 400 MHz) δ=8.46 (d, J=9.9 Hz, 1H), 8.13 (s, 1H), 7.64 (s, 1H), 7.61-7.56 (m, 1H), 7.47 (d, J=10.0 Hz, 1H), 5.18 (s, 2H), 4.36 (br d, J=12.1 Hz, 1H), 4.12 (br d, J=11.9 Hz, 1H), 3.80 (s, 3H), 3.08 (br d, J=11.4 Hz, 1H), 2.89-2.71 (m, 2H), 2.65-2.57 (m, 1H), 2.39 (dd, J=2.4, 10.8 Hz, 1H), 2.35 (s, 3H), 0.96 (s, 9H), 1H not observed (NH); LC/MS (ESI): 464.2 [M+H]⁺.

### Example 26. Synthesis of 2-(6-(4,7-diazaspiro[2.5]octan-7-yl)pyridazin-3-yl)-6-(2,4-dimethylthiazol-5-yl)-4-fluoroisoindolin-1-one (Compound No. 300)

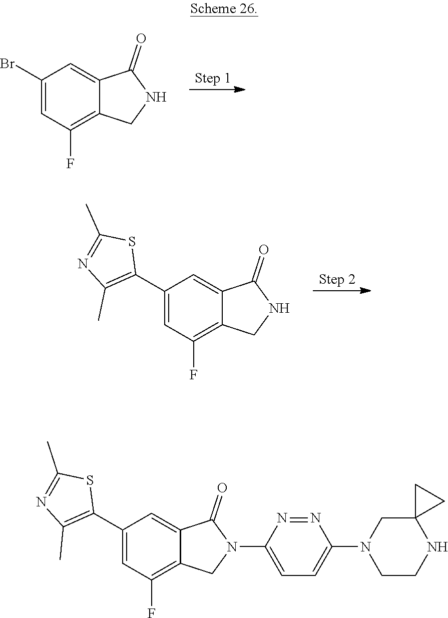

Step 1: 6-Bromo-4-fluoroisoindolin-1-one (457.9 mg, 2.0 mmol, 1.0 eq), 2,4-dimethyl-5-(4,4,5,5-tetramethyl-1,3,2-dioxaborolan-2-yl)thiazole) (526.1 mg, 2.20 mmol, 1.1 eq), aqueous potassium carbonate (2 M, 8 mL) and PdCl₂(dppf)·CH₂Cl₂ adduct (146.3 mg, 0.2 mmol, 0.1 eq) were charged to a three-neck 100 mL round bottom flask under nitrogen. Anhydrous dioxane (9 mL) was added and the reaction mixture was heated to 85° C. overnight. Then the mixture was cooled, filtered through a pad of Celite, and the filter cake was rinsed with EA (100 mL). The filtrate was washed with brine and the organic layer was collected, dried over sodium sulfate, and concentrated in vacuo. The crude product was purified by silica gel column chromatography with DCM/MeOH 20/1, v/v to afford 6-(2,4-dimethylthiazol-5-yl)-4-fluoroisoindolin-1-one (273.6 mg, 57%) as a brown solid. LC/MS (ESI): 263.1 [M+H]⁺.

Step 2: To a round bottom flask charged with 6-(2,4-dimethylthiazol-5-yl)-4-fluoroisoindolin-1-one (37.4 mg, 0.15 mmol, 1.0 eq), tert-butyl 7-(6-bromopyridazin-3-yl)-4,7-diazaspiro[2.5]octane-4-carboxylate (55.2 mg, 0.15 mmol, 1.1 eq), Cs₂CO₃ (148.9 mg, 0.46 mmol, 3.0 eq), and XantPhos Pd G4 (14.2 mg, 0.015 mmol, 0.1 eq) was added 1,4-dioxane (4 mL). The reaction mixture was degassed with argon for 5 min and was then heated to 90° C. for 8 h. The reaction mixture was cooled to RT and was then diluted with EA. The organic phase was washed with brine, dried over sodium sulfate, and concentrated under vacuum. The crude product was purified by silica gel column chromatography with DCM/MeOH 20/1, v/v to obtain 5-(1-(tetrahydro-2H-pyran-2-yl)-1H-pyrazol-4-yl)isoindolin-1-one (42.2 mg), which was dissolved HCl in dioxane (2 mL, 4.0 mol/L). The reaction was stirred at RT for 1 h and was then concentrated. The resulting residue was purified by RP HPLC using MeCN and H₂O as eluent to provide 2-(6-(4,7-diazaspiro[2.5]octan-7-yl)pyridazin-3-yl)-6-(2,4-dimethylthiazol-5-yl)-4-fluoroisoindolin-1-one (13.59 mg, 20.1%) as a white solid.

¹HNMR (400 MHz, DMSO-d₆) δ=8.44 (d, J=9.9 Hz, 1H), 8.15 (d, J=1.0 Hz, 1H), 7.70-7.65 (m, 1H), 7.63 (s, 1H), 7.44 (d, J=10.0 Hz, 1H), 5.24 (s, 2H), 3.58-3.53 (m, 2H), 3.44 (s, 2H), 2.89 (br d, J=4.5 Hz, 2H), 2.66 (s, 3H), 2.43 (s, 3H), 0.51 (br d, J=9.8 Hz, 4H); LC/MS (ESI): 451.1 [M+H]⁺.

### Example 27. Synthesis of 6-(1,3-dimethyl-1H-pyrazol-4-yl)-2-(6-(((1R,3s,5S)-1,5-dimethyl-8-azabicyclo[3.2.1]octan-3-yl)oxy)pyridazin-3-yl)isoindolin-1-one (Compound No. 416)

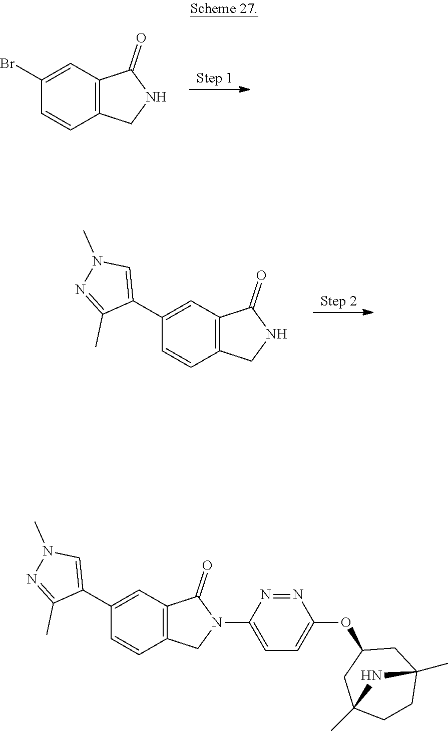 [SMILES: O=C1NCc2ccc(Br)cc21 ; Cc1nn(C)cc1-c1ccc2c(c1)C(=O)NC2 ; Cc1nn(C)cc1-c1ccc2c(c1)C(=O)N(c1ccc(O[C@H]3C[C@]4(C)CC[C@](C)(C3)N4)nn1)C2]

Step 1: 6-Bromoisoindolin-1-one (426.0 mg, 2.0 mmol, 1.0 eq), 1,3-dimethyl-4-(4,4,5,5-tetramethyl-1,3,2-dioxaborolan-2-yl)-1H-pyrazole (488 mg, 2.20 mmol, 1.1 eq), aqueous potassium carbonate (2 M, 8 mL) and PdCl₂(dppf)·CH₂Cl₂ adduct (146.3 mg, 0.2 mmol, 0.1 eq) were charged to a three-neck 100 mL round bottom flask under nitrogen. Anhydrous dioxane (9 mL) was added and the reaction mixture was heated to 85° C. overnight. Then the mixture was cooled, filtered through a pad of Celite, and the filter cake was rinsed with EA (100 mL). The filtrate was washed with brine and the organic layer was collected, dried over sodium sulfate, and concentrated in vacuo. The crude product was purified by silica gel column chromatography with DCM/MeOH 20/1, v/v to afford 6-(1,3-dimethyl-1H-pyrazol-4-yl)isoindolin-1-one (335 mg, 68%) as a brown solid. LC/MS (ESI): 228.1 [M+H]⁺.

Step 2: To a round bottom flask charged with 6-(1,3-dimethyl-1H-pyrazol-4-yl)isoindolin-1-one (34.2 mg, 0.15 mmol, 1.0 eq), tert-butyl (1R,3s,5S)-3-((6-bromopyridazin-3-yl)oxy)-1,5-dimethyl-8-azabicyclo[3.2.1]octane-8-carboxylate (61.8 mg, 0.15 mmol, 1.1 eq), Cs₂CO₃ (148.9 mg, 0.46 mmol, 3.0 eq), and XantPhos Pd G4 (14.2 mg, 0.015 mmol, 0.1 eq) was added 1,4-dioxane (4 mL). The reaction mixture was degassed with argon for 5 min and was then heated to 90° C. for 8 h. The reaction mixture was cooled down to RT and was then diluted with EA. The organic phase was washed with brine, dried over sodium sulfate, and concentrated under vacuum. The crude product was purified by silica gel column chromatography with DCM/MeOH 20/1, v/v, and was then dissolved HCl in dioxane (2 mL, 4.0 mol/L). The reaction was stirred at RT for 1 h and was then concentrated. The resulting residue was purified by RP HPLC using MeCN and H₂O as eluent to provide 6-(1,3-dimethyl-1H-pyrazol-4-yl)-2-(6-(((1R,3s,5S)-1,5-dimethyl-8-azabicyclo[3.2.1]octan-3-yl)oxy)pyridazin-3-yl)isoindolin-1-one (16.84 mg, 23.4%) as a white solid.

¹H NMR (DMSO-d₆, 400 MHz) δ=8.73 (d, J=9.6 Hz, 1H), 8.20 (br d, J=4.5 Hz, 1H), 8.06 (s, 1H), 7.81-7.77 (m, 2H), 7.76-7.73 (m, 1H), 7.33 (d, J=9.5 Hz, 1H), 5.49 (s, 1H), 5.16 (s, 2H), 3.81 (s, 3H), 2.34 (s, 3H), 2.33-2.29 (m, 2H), 2.03-1.93 (m, 2H), 1.72 (br d, J=2.5 Hz, 2H), 1.66-1.56 (m, 2H), 1.34 (br s, 6H); LC/MS (ESI): 459.3 [M+H]⁺.

### Example 28. Synthesis of 6-(1,3-dimethyl-1H-pyrazol-4-yl)-2-(6-(((1R,3s,5S)-1,5-dimethyl-8-azabicyclo[3.2.1]octan-3-yl)oxy)pyridazin-3-yl)-7-fluoro-2,3-dihydro-1H-pyrrolo[3,4-c]pyridin-1-one (Compound No. 116)

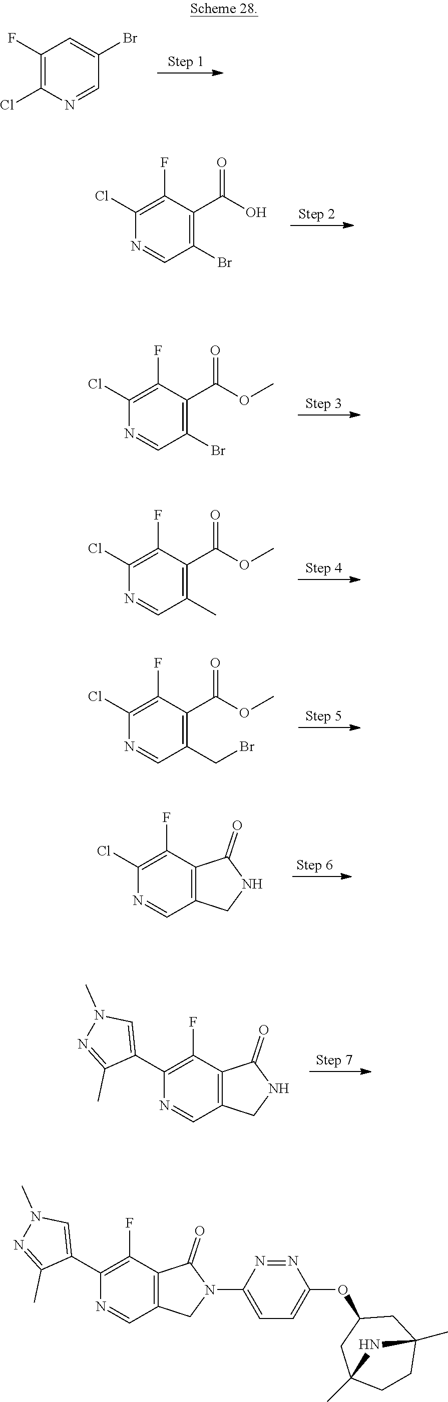

Step 1: LDA (48 mL, 2.0 mol in THF) was added dropwise into a solution of 5-bromo-2-chloro-3-fluoropyridine (10 g, 47.52 mmol) in THF (200 mL) at −78° C. under N₂ and the reaction mixture was stirred at −78° C. for 2 h. The mixture was poured into the CO₂ (solid) in THF. The reaction mixture was then concentrated under vacuum. The pH value of the residue was adjusted to 3 with 2 μM HCl. The resulting mixture was extracted with EA, washed with brine, dried over MgSO₄, and concentrated under vacuum to afford the desired product (12 g, crude) as a white solid.

Step 2: To a solution of 5-bromo-2-chloro-3-fluoroisonicotinic acid (12 g, 51.2 mmol) and K₂CO₃ (5.7 g, 41.7 mmol) in DMF (80 mL), Mel (3.4 mL, 54.7 mmol) was added portionwise; and the reaction mixture was stirred at RT for 16 h. The reaction mixture was poured into H₂O. The aqueous layer was extracted EA. The organic layer was washed with brine, dried over MgSO₄, filtered, and concentrated. The crude product was purified by silica gel column chromatography with PE/EA 10/1, v/v to obtain methyl 5-bromo-2-chloro-3-fluoroisonicotinate (10 g, 79%).

Step 3: To the solution of 5-bromo-2-chloro-3-fluoroisonicotinate (2500 mg, 9.4 mmol) and 2,4,6-trimethyl-1,3,5,2,4,6-trioxatriborinane in dioxane (25 mL), was added potassium carbonate (2600 mg, 18.8 mmol) and PdCl₂(dppf) (500 mg, 0.94 mmol); and the reaction mixture was stirred under N₂ at 110° C. for 1 h. Then the mixture was cooled, filtered through a pad of Celite and the filter cake was washed with EA. The filtrate was washed with brine and the organic layer was collected, dried over sodium sulfate, and concentrated in vacuo. The crude product was purified by silica gel column chromatography with PE/EA 10/1, v/v to afford methyl 2-chloro-3-fluoro-5-methylisonicotinate (673 mg, 35.3%).

Step 4: To a solution of methyl 2-chloro-3-fluoro-5-methylisonicotinate (320 mg, 1.6 mmol) and NBS (285 mg, 1.6 mmol) in CCl₄ (10 mL), was added BPO (40 mg, 0.16 mmol); and the reaction mixture was stirred under N₂ at 80° C. for 16 h. The mixture was cooled to room temperature and was then poured into H₂O. The aqueous layer was extracted EA. The organic layer was washed with brine, dried over MgSO₄, filtered, and concentrated. The crude product was purified by silica gel column chromatography with PE/EA 5/1, v/v to afford methyl 5-(bromomethyl)-2-chloro-3-fluoroisonicotinate (356 mg, 80%).

Step 5: Methyl 5-(bromomethyl)-2-chloro-3-fluoroisonicotinate (356 mg, 1.26 mmol) was dissolved in THF (5 mL) and NH₄OH (5 mL) was added; and the reaction was stirred at RT for 3 h. The reaction mixture was filtered and concentrated under vacuum. The crude product was used directly in the next step.

Step 6: 6-Chloro-7-fluoro-2,3-dihydro-1H-pyrrolo[3,4-c]pyridin-1-one (186.0 mg, 1.0 mmol, 1.0 eq), 1,3-dimethyl-4-(4,4,5,5-tetramethyl-1,3,2-dioxaborolan-2-yl)-1H-pyrazole (312 mg, 1.5 mmol, 1.5 eq), aqueous potassium carbonate (2 M, 2 mL) and PdCl₂(dppf)·CH₂Cl₂ adduct (73.2 mg, 0.1 mmol, 0.1 eq) were charged to a three-neck 100 mL round bottom flask under nitrogen and then anhydrous dioxane (9 mL) was added and the reaction mixture was heated to 85° C. overnight. The mixture was cooled, filtered through a pad of Celite, and the filter cake was rinsed with EA (100 mL). The filtrate was washed with brine and the organic layer was collected, dried over sodium sulfate, and concentrated in vacuo. The crude product was purified by silica gel column chromatography with DCM/MeOH 20/1, v/v to afford 6-(1,3-dimethyl-1H-pyrazol-4-yl)-7-fluoro-2,3-dihydro-1H-pyrrolo[3,4-c]pyridin-1-one (100.8 mg, 41.2%) as a brown solid. LC/MS (ESI): 247.1 [M+H]⁺.

Step 7: To a round bottom flask charged with 6-(1,3-dimethyl-1H-pyrazol-4-yl)-7-fluoro-2,3-dihydro-1H-pyrrolo[3,4-c]pyridin-1-one (36.9 mg, 0.15 mmol, 1.0 eq), tert-butyl (S)-4-(6-bromopyridazin-3-yl)-2-isopropylpiperazine-1-carboxylate (57.6 mg, 0.15 mmol, 1.1 eq), Cs₂CO₃ (148.9 mg, 0.46 mmol, 3.0 eq), and XantPhos Pd G4 (14.2 mg, 0.015 mmol, 0.1 eq) was added 1,4-dioxane (4 mL). The reaction mixture was degassed with argon for 5 min and was then heated to 90° C. for 8 h. The reaction mixture was cooled to RT and was diluted with EA. The organic phase was washed with brine, dried over sodium sulfate, and concentrated under vacuum. The crude product was purified by silica gel column chromatography with DCM/MeOH 20/1, v/v, and was then dissolved HCl in dioxane (2 mL, 4.0 mol/L). The reaction was stirred at RT for 1 h and was then concentrated. The resulting residue was purified by RP HPLC using MeCN and H₂O as eluent to provide 6-(1,3-dimethyl-1H-pyrazol-4-yl)-2-(6-(((1R,3s,5S)-1,5-dimethyl-8-azabicyclo[3.2.1]octan-3-yl)oxy)pyridazin-3-yl)-7-fluoro-2,3-dihydro-1H-pyrrolo[3,4-c]pyridin-1-one (3.93 mg, 5.6%) as a white solid.

¹HNMR (400 MHz, METHANOL-d₄) δ=8.72-8.67 (m, 2H), 8.44 (s, 1H), 8.00 (d, J=3.3 Hz, 1H), 7.15 (d, J=9.6 Hz, 1H), 5.58-5.44 (m, 1H), 5.18 (s, 2H), 3.81 (s, 3H), 2.45-2.38 (m, 5H), 2.16 (br d, J=8.3 Hz, 2H), 1.90-1.82 (m, 2H), 1.68 (br t, J=12.0 Hz, 2H), 1.40 (s, 6H); LC/MS (ESI): 478.2 [M+H]⁺.

### Example 29. Synthesis of 4-chloro-6-(1,3-dimethyl-1H-pyrazol-4-yl)-2-(6-(((1R,3s,5S)-1,5-dimethyl-8-azabicyclo[3.2.1]octan-3-yl)oxy)pyridazin-3-yl)-2,3-dihydro-1H-pyrrolo[3,4-c]pyridin-1-one (Compound No. 93)

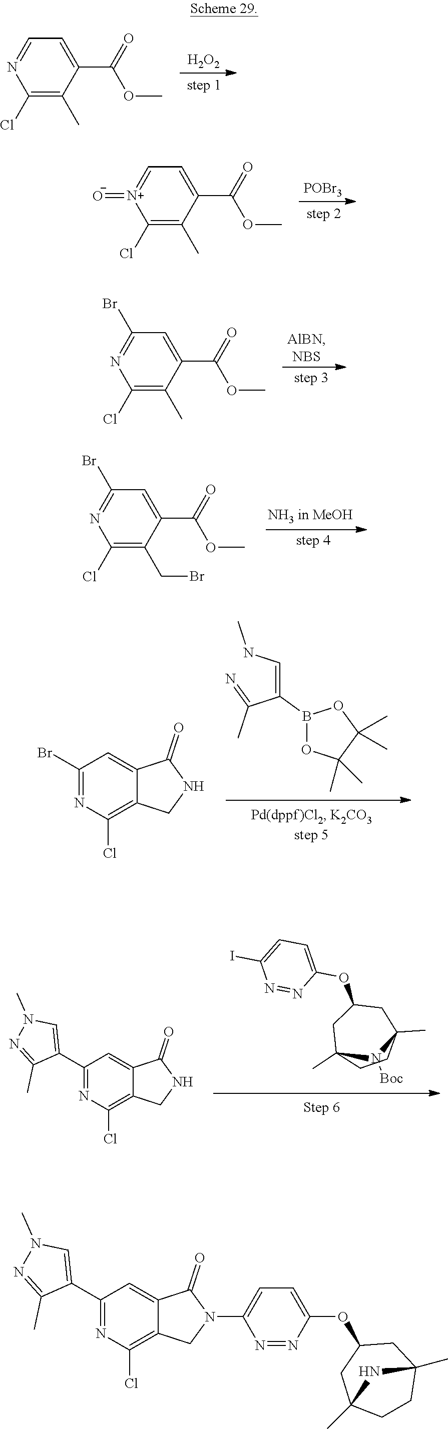

Step 1: To methyl 2-chloro-3-methylisonicotinate (5000 mg, 27.03 mmol) in TFA (50 mL), H₂O₂(42.16 mL, 351.35 mmol) was added and the mixture was stirred for 4 h. The mixture was cooled to RT and quenched with aqueous Na₂SO₃. The mixture was concentrated under vacuum and the residue was redissolved in EA (300 mL). The mixture was washed with brine and the organic layer was collected, dried over sodium sulfate, and concentrated in vacuo. The crude product was purified by silica gel column chromatography with PE/EtOAc to provided 2-chloro-4-(methoxycarbonyl)-3-methylpyridine 1-oxide (3000 mg, 14.85 mmol, 54.95%). LC/MS (ESI): 202.0 [M+H]⁺.

Step 2: POBr3 (6398.79 mg, 22.32 mmol) was added to the solution of 2-chloro-4-(methoxycarbonyl)-3-methylpyridine 1-oxide (3000 mg, 14.88 mmol) in toluene (30 mL) at RT and the reaction mixture was stirred at 80° C. for 4 h. The reaction mixture was cooled to RT and was quenched with aqueous Na₂CO₃. This mixture was extracted with EA (3×100 mL) and the combined extracts were dried over Na₂SO₄, filtered, and concentrated under reduced pressure. The crude product was purified by silica gel chromatography eluted with PE: EtOAc=2:1, to give methyl methyl 6-bromo-2-chloro-3-methylisonicotinate (1500 mg, 5.67 mmol, 38.11%) as a white solid. LC/MS (ESI): 263.9 [M+H]⁺.

Step 3: Methyl 6-bromo-2-chloro-3-methylpyridine-4-carboxylate (1500 mg, 5.67 mmol), NBS (1513.80 mg, 8.50 mmol) and AIBN (0.17 mL, 1.13 mmol) were combined with carbon tetrachloride (40 mL); and the reaction mixture was heated to 80° C. for 3 h. The reaction mixture was cooled to RT and concentrated under vacuum. The resulting residue was purified by silica gel column chromatography with PE/EA to afford ethyl methyl 6-bromo-3-(bromomethyl)-2-chloropyridine-4-carboxylate (1150 mg, 3.35 mmol, 59.05%) as a brown solid. LC/MS (ESI): 341.8 [M+H]⁺.

Step 4: Methyl 6-bromo-3-(bromomethyl)-2-chloropyridine-4-carboxylate (1150 mg, 3.35 mmol) was suspended in 28% aqueous ammonium hydroxide and the mixture was stirred at RT for 8 h. Then the mixture was filtered, and the residue was washed with water and methanol. The solid was collected, dried under vacuum and used in the next step without further purification. LC/MS (ESI): 246.9 [M+H]⁺.

Step 5: 6-Bromo-4-chloro-2,3-dihydro-1H-pyrrolo[4,3-c]pyridin-1-one (550 mg, 2.22 mmol), 2,5-dimethyl-4-(4,4,5,5-tetramethyl-1,3,2-dioxaborolan-2-yl)-1,2,3-triazole (545.35 mg, 2.44 mmol), K₂CO₃ (921.41 mg, 6.67 mmol)) and Pd(dppf)Cl₂ (162.62 mg, 0.22 mmol) were charged into a 100 mL round bottom flask. The flask was placed under nitrogen and anhydrous dioxane (9 mL) and water (1 mL) were added and the mixture was heated to 85° C. overnight. Then the mixture was cooled, filtered through a pad of Celite, and washed with EA (50 mL). The filtrate was washed with washed with brine and the organic layer was collected, dried over sodium sulfate, and concentrated under vacuo. The crude product was purified by silica gel column chromatography with DCM/MeOH 20/1, v/v to obtain 4-chloro-6-(2,5-dimethyl-1,2,3-triazol-4-yl)-2,3-dihydro-1H-pyrrolo[4,3-c]pyridin-1-one (400 mg, 1.52 mmol, 68.26%) as a brown solid. LC/MS (ESI): 263.1 [M+H]⁺.

Step 6: To a round bottom flask charged with 4-chloro-6-(1,3-dimethylpyrazol-4-yl)-2,3-dihydro-1H-pyrrolo[4,3-c]pyridin-1-one (120 mg, 0.46 mmol), 2-methylpropan-2-yl(1S,3s,5R)-3-[(6-iodo-1,2-diazin-3-yl)oxy]-1,5-dimethyl-8-azabicyclo[3.2.1]octane-8-carboxylate (230.80 mg, 0.50 mmol), Cs₂CO₃ (446.50 mg, 1.37 mmol), and XantPhos Pd G4 (43.96 mg, 0.05 mmol) was added 1,4-dioxane (4 mL). The reaction mixture was degassed with argon for 5 min and was then heated to 90° C. for 8 h. The reaction mixture was cooled to RT and then diluted with EA. The organic phase was washed with brine, dried over sodium sulfate, and concentrated under vacuum. The crude product was purified by silica gel column chromatography with DCM/MeOH 20/1, v/v to obtain 2-methylpropan-2-yl (1S,3s,5R)-3-({6-[4-chloro-6-(1,3-dimethylpyrazol-4-yl)-1-oxo-2,3-dihydro-1H-pyrrolo[4,3-c]pyridin-2-yl]-1,2-diazin-3-yl}oxy)-1,5-dimethyl-8-azabicyclo[3.2.1]octane-8-carboxylate (40 mg), which was dissolved HCl in dioxane (2 mL, 4.0 mol/L). The reaction was stirred at RT for 1 h and was then concentrated. The resulting residue was purified by RP HPLC using MeCN and H₂O as eluent to provide 4-chloro-6-(1,3-dimethylpyrazol-4-yl)-2-(6-{[(1S,3s,5R)-1,5-dimethyl-8-azabicyclo[3.2.1]octan-3-yl]oxy}-1,2-diazin-3-yl)-2,3-dihydro-1H-pyrrolo[4,3-c]pyridin-1-one (10.3 mg, 0.02 mmol, 4.53%) as a white solid.

¹H NMR (DMSO-d₆, 400 MHz) δ=8.96 (br d, J=8.8 Hz, 1H), 8.85 (br d, J=9.3 Hz, 1H), 8.70 (d, J=9.6 Hz, 1H), 8.47 (s, 1H), 7.94 (s, 1H), 7.41 (d, J=9.6 Hz, 1H), 5.61-5.47 (m, 1H), 5.16 (s, 2H), 3.82 (s, 3H), 2.48-2.42 (m, 5H), 2.16 (br d, J=8.0 Hz, 2H), 1.95-1.75 (m, 4H), 1.45 (s, 6H); LC/MS (ESI): 494.1 [M+H]⁺.

### Example 30. Synthesis of 6-(1,3-dimethyl-1H-pyrazol-4-yl)-2-(6-(7-ethyl-2,7-diazaspiro[4.4]nonan-2-yl)pyridazin-3-yl)-5-fluoroisoindolin-1-one (Compound No. 414)

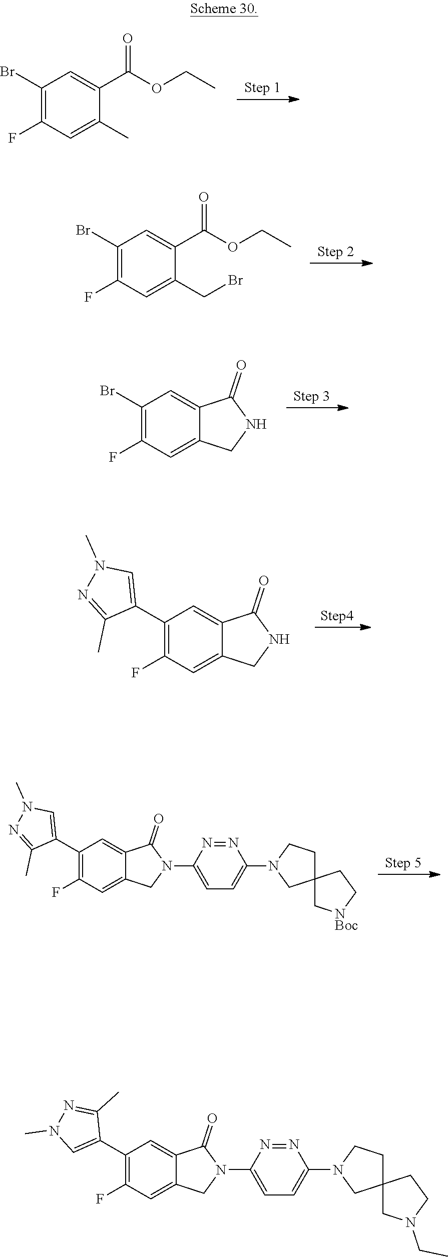 [SMILES: CCOC(=O)c1cc(Br)c(F)cc1C ; CCOC(=O)c1cc(Br)c(F)cc1CBr ; O=C1NCc2cc(F)c(Br)cc21 ; Cc1nn(C)cc1-c1cc2c(cc1F)CNC2=O ; Cc1nn(C)cc1-c1cc2c(cc1F)CN(c1ccc(N3CCC4(CCN(C(=O)OC(C)(C)C)C4)C3)nn1)C2=O ; CCN1CCC2(CCN(c3ccc(N4Cc5cc(F)c(-c6cn(C)nc6C)cc5C4=O)nn3)C2)C1]

Step 1: Ethyl 5-bromo-4-fluoro-2-methylbenzoate (2.60 g, 10.0 mmol, 1.0 eq), N-bromosuccinimide (2.49 g, 14.0 mmol, 1.4 eq) and benzoyl peroxide (242. mg, 1.0 mmol, 0.1 eq) were combined with carbon tetrachloride (150 mL). The reaction mixture was heated to 80° C. and stirred for 3 h. The reaction mixture was cooled to RT and concentrated under vacuum. The resulting residue was purified by silica gel column chromatography with PE/EA to afford ethyl 5-bromo-2-(bromomethyl)-4-fluorobenzoate (2.61 g, 77%) as a brown solid. LC/MS (ESI): 338.9, 340.9, 342.9 [M+H]⁺.

Step 2: Ethyl 5-bromo-2-(bromomethyl)-4-fluorobenzoate (2.61 g, 7.7 mmol, 1.0 eq) was suspended in 28% aqueous ammonium hydroxide and the mixture was stirred at RT for 8 h. Then the mixture was filtered, and the residue was washed with water and methanol. The solid was collected, dried under vacuum and used in the next step without further purification. LC/MS (ESI): 230.0, 232.0 [M+H]⁺.

Step 3: 6-Bromo-5-fluoroisoindolin-1-one (456.0 mg, 2.0 mmol, 1.0 eq), 1,3-dimethyl-4-(4,4,5,5-tetramethyl-1,3,2-dioxaborolan-2-yl)-1H-pyrazole (488 mg, 2.20 mmol, 1.1 eq), aqueous potassium carbonate (2 M, 8 mL) and PdCl₂(dppf)·CH₂Cl₂ adduct (146.3 mg, 0.2 mmol, 0.1 eq) were charged to a three-neck 100 mL round bottom flask under nitrogen and anhydrous dioxane (9 mL) was added and the mixture was heated to 85° C. overnight. The mixture was cooled, filtered through a pad of Celite, and the filter cake was rinsed with EA (100 mL). The filtrate was washed with brine and the organic layer was collected, dried over sodium sulfate, and concentrated in vacuo. The crude product was purified by silica gel column chromatography with DCM/MeOH 20/1, v/v to afford 6-(1,3-dimethyl-1H-pyrazol-4-yl)-5-fluoroisoindolin-1-one (298.9 mg, 61%) as a brown solid. LC/MS (ESI): 246.1 [M+H]⁺.

Step 4: To a round bottom flask charged with 6-(1,3-dimethyl-1H-pyrazol-4-yl)isoindolin-1-one (34.2 mg, 0.15 mmol, 1.0 eq), tert-butyl 7-(6-bromopyridazin-3-yl)-2,7-diazaspiro[4.4]nonane-2-carboxylate (57.3 mg, 0.15 mmol, 1.1 eq), Cs₂CO₃ (148.9 mg, 0.46 mmol, 3.0 eq), and XantPhos Pd G4 (14.2 mg, 0.015 mmol, 0.1 eq) was added 1,4-dioxane (4 mL). The reaction mixture was degassed with argon for 5 min and was then heated to 90° C. for 8 h. The reaction mixture was cooled to RT and diluted with EA. The organic phase was washed with brine, dried over sodium sulfate, and concentrated under vacuum. The crude product was purified by silica gel column chromatography with DCM/MeOH 20/1, v/v to afford tert-butyl 7-(6-(6-(1,3-dimethyl-1H-pyrazol-4-yl)-5-fluoro-1-oxoisoindolin-2-yl)pyridazin-3-yl)-2,7-diazaspiro[4.4]nonane-2-carboxylate (69 mg, 77%) as a brown solid. LC/MS (ESI): 578.3 [M+H]⁺.

Step 5: To a round bottom flask charged with tert-butyl 7-(6-(6-(1,3-dimethyl-1H-pyrazol-4-yl)-5-fluoro-1-oxoisoindolin-2-yl)pyridazin-3-yl)-2,7-diazaspiro[4.4]nonane-2-carboxylate (69 mg, 0.12 mmol, 1.0 eq) and acetaldehyde (0.3 mL, 1.52 mmol, 12.0 eq) was added MeOH (4 mL); and the reaction mixture was stirred at 0° C. for 1 h. Then NaBH₃CN (37.24 mg, 0.76 mmol, 6.0 eq) was added and the reaction mixture was stirred at RT for 1 h. The reaction was concentration and the resulting residue was purified by RP-HPLC using MeCN and H₂O as eluent to provide 6-(1,3-dimethyl-1H-pyrazol-4-yl)-2-(6-(7-ethyl-2,7-diazaspiro[4.4]nonan-2-yl)pyridazin-3-yl)-5-fluoroisoindolin-1-one (9.02 mg, 0.01 mmol, 12.6%) as a white solid.

¹H NMR (400 MHz, METHANOL-d₄) S: 8.58 (d, J=9.9 Hz, 1H), 7.85 (d, J=6.9 Hz, 1H), 7.80 (d, J=1.8 Hz, 1H), 7.51 (d, J=9.9 Hz, 1H), 7.07 (d, J=9.9 Hz, 1H), 5.14 (s, 2H), 3.90 (s, 3H), 3.64-3.50 (m, 4H), 3.10 (br t, J=6.8 Hz, 2H), 3.05-2.97 (m, 2H), 2.89 (q, J=7.3 Hz, 2H), 2.33 (s, 3H), 2.20-2.10 (m, 2H), 2.04 (dt, J=2.6, 7.1 Hz, 2H), 1.25 (t, J=7.3 Hz, 3H); LC/MS (ESI): 476.3 [M+H]⁺.

### Example 31. Synthesis of 5-(3-methyl-1,2,4-thiadiazol-5-yl)-2-(6-((2,2,6,6-tetramethylpiperidin-4-yl)oxy)pyridazin-3-yl)isoindolin-1-one (Compound No. 451)

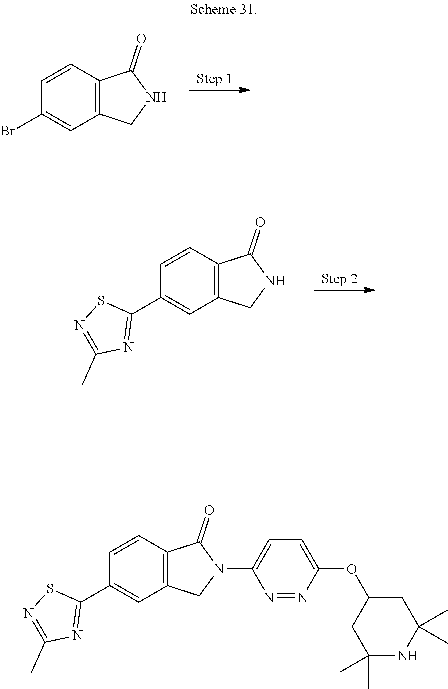 [SMILES: O=C1NCc2cc(Br)ccc21 ; Cc1nsc(-c2ccc3c(c2)CNC3=O)n1 ; Cc1nsc(-c2ccc3c(c2)CN(c2ccc(OC4CC(C)(C)NC(C)(C)C4)nn2)C3=O)n1]

Step 1: 5-Bromoisoindolin-1-one (315 mg, 1.5 mmol, 1.5 eq), 4,4,4′,4′,5,5,5′,5′-octamethyl-2,2′-bi(1,3,2-dioxaborolane (762 mg, 3.0 mmol, 3.0 eq), potassium acetate (300 mg, 3.0 mmol, 3.0 eq) and PdCl₂(dppf)·CH₂Cl₂ adduct (109.3 mg, 0.15 mmol, 0.1 eq) were charged to a three-neck 100 mL round bottom flask under nitrogen and anhydrous dioxane (9 mL) was added and the mixture was heated to 90° C. When full conversion was achieved, the reaction mixture was cooled to room temperature. Then 5-bromo-3-methyl-1,2,4-thiadiazole (177.3 mg, 1.0 mmol, 1.0 eq), aqueous potassium carbonate (2 M, 2 mL) and PdCl₂(dppf)·CH₂Cl₂ adduct (109.3 mg, 0.15 mmol, 0.1 eq) were added and the reaction mixture was heated to 90° C. When full conversion was achieved, the mixture was cooled, filtered through a pad of Celite, and the filter cake was rinsed with EA (100 mL). The filtrate was washed with brine and the organic layer was collected, dried over sodium sulfate, and concentrated in vacuo. The crude product was purified by silica gel column chromatography with DCM/MeOH 20/1, v/v to afford 5-(3-methyl-1,2,4-thiadiazol-5-yl)isoindolin-1-one (71.9 mg, 31%) as a brown solid. LC/MS (ESI): 232.1 [M+H]⁺.

Step 2: To a round bottom flask charged with 5-(3-methyl-1,2,4-thiadiazol-5-yl)isoindolin-1-one (34.8 mg, 0.15 mmol, 1.0 eq), 3-bromo-6-((2,2,6,6-tetramethylpiperidin-4-yl)oxy)pyridazine (50.0 mg, 0.16 mmol, 1.1 eq), Cs₂CO₃ (148.9 mg, 0.46 mmol, 3.0 eq), and XantPhos Pd G4 (14.2 mg, 0.015 mmol, 0.1 eq) was added 1,4-dioxane (4 mL). The reaction mixture was degassed with argon for 5 min and was then heated to 90° C. for 8 h. The reaction mixture was cooled to RT and diluted with EA. The organic phase was washed with brine, dried over sodium sulfate, and concentrated under vacuum. The crude product was purified by silica gel column chromatography with DCM/MeOH 20/1, v/v. The resulting residue was further purified by RP HPLC using MeCN and H₂O as eluent to provide 5-(3-methyl-1,2,4-thiadiazol-5-yl)-2-(6-((2,2,6,6-tetramethylpiperidin-4-yl)oxy)pyridazin-3-yl)isoindolin-1-one (20.68 mg, 29.7%) as a white solid.

¹H NMR (400 MHz, DMSO-d₆) δ=9.16 (d, J=1.9 Hz, 1H), 8.74 (dd, J=2.6, 9.4 Hz, 1H), 8.71-8.67 (m, 1H), 8.47-8.44 (m, 1H), 8.42 (d, J=2.0 Hz, 1H), 8.16 (s, 1H), 5.23 (d, J=5.5 Hz, 2H), 4.13 (br s, 1H), 4.06-4.03 (m, 1H), 3.90 (s, 3H), 3.54-3.44 (m, 1H), 2.76 (br d, J=14.6 Hz, 1H), 2.44-2.34 (m, 1H), 2.22-2.14 (m, 1H), 2.11-2.07 (m, 1H), 2.06-1.96 (m, 2H), 1.85-1.70 (m, 1H), 1.58 (br d, J=8.0 Hz, 1H); LC/MS (ESI): 402.2 [M+H]⁺.

### Example 32. Synthesis of 3-(1,3-dimethyl-1H-pyrazol-4-yl)-6-(6-(((1R,3s,5S)-1,5-dimethyl-8-azabicyclo[3.2.1]octan-3-yl)(methyl)amino)pyridazin-3-yl)-6,7-dihydro-5H-pyrrolo[3,4-b]pyrazin-5-one (Compound No. 452)

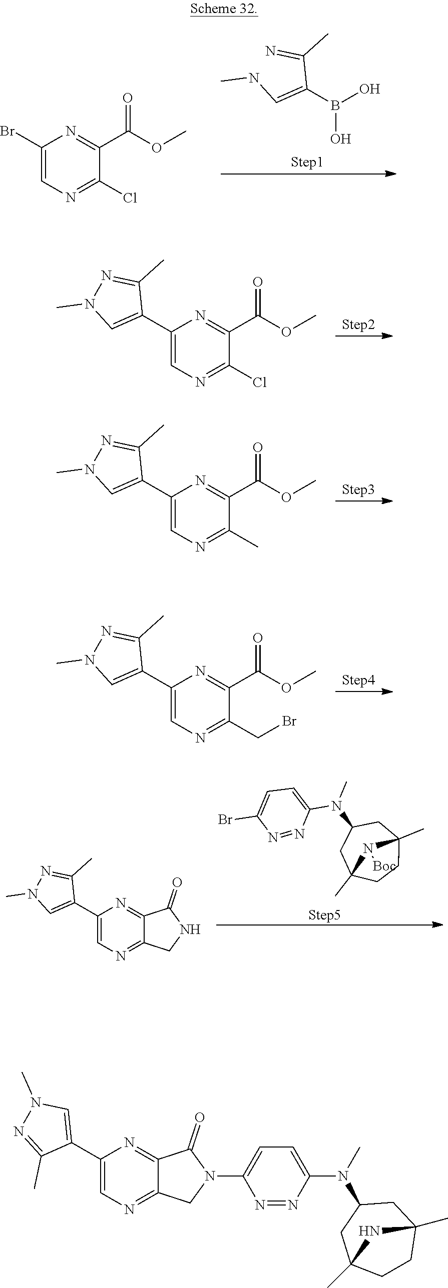

Step 1: Methyl 6-bromo-3-chloropyrazine-2-carboxylate (400 mg, 1.59 mmol, 1.0 eq), (2,5-dimethylpyrazol-3-yl)boranediol (222.52 mg, 1.59 mmol, 1.0 eq), sodium carbonate (505.57 mg, 4.77 mmol, 3.0 eq) and PdCl₂(dppf)·CH₂Cl₂ adduct (116.28 mg, 0.16 mmol, 0.1 eq) were charged to a three-neck 100 mL round bottom flask under nitrogen and anhydrous dioxane (9 mL) and water (1 mL) were added and the reaction mixture was heated to 85° C. overnight. Then the mixture was cooled, filtered through a pad of Celite, and the filter cake was rinsed with EA (100 mL). The filtrate was washed with brine and the organic layer was collected, dried over sodium sulfate, and concentrated in vacuo. The crude product was purified by silica gel column chromatography with PE/EA 5/1, v/v to afford methyl 3-chloro-6-(1,3-dimethylpyrazol-4-yl)pyrazine-2-carboxylate (300 mg, 70.72%) LC/MS (ESI): 267.0 [M+H]⁺.

Step 2: Methyl 3-chloro-6-(1,3-dimethylpyrazol-5-yl)pyrazine-2-carboxylate (500 mg, 1.87 mmol, 1.2 eq), 2,4,6-trimethyl-1,3,5,2,4,6-trioxatriborinane (1.29 mL, 2.25 mmol, 1.0 eq), potassium carbonate (777.31 mg, 5.62 mmol, 3.0 eq) and PdCl₂(dppf)·CH₂Cl₂ adduct (137.05 mg, 0.19 mmol, 0.1 eq) were charged to a three-neck 100 mL round bottom flask under nitrogen. Anhydrous dioxane (10 mL) was added and the reaction mixture was heated to 110° C. 3 h. Then the mixture was cooled, filtered through a pad of Celite, and the filter cake was rinsed with EA(100 mL). The filtrate was washed with brine and the organic layer was collected, dried over sodium sulfate, and concentrated in vacuo. The crude product was purified by silica gel column chromatography with PE/EA 5/1, v/v to afford methyl 6-(1,3-dimethylpyrazol-5-yl)-3-methylpyrazine-2-carboxylate (430 mg, 93.13%). LC/MS (ESI): 247.1 [M+H]⁺.

Step 3: Methyl 6-(1,3-dimethyl-1H-pyrazol-4-yl)-3-methylpyrazine-2-carboxylate (430 mg, 1.75 mmol, 1.0 eq), N-bromosuccinimide (371.67 mg, 2.10 mmol, 1.2 eq) and benzoyl peroxide (129.17 mg, 0.79 mmol, 0.45 eq) were combined with carbon tetrachloride (10 mL) and the reaction mixture was heated to 80° C. and stirred for 16 h. The reaction mixture was cooled to RT and concentrated under vacuum. The resulting residue was purified by silica gel column chromatography with PE/EA to afford methyl 3-(bromomethyl)-6-(1,3-dimethyl-1H-pyrazol-4-yl)pyrazine-2-carboxylate (440 mg, 77.32%) as a brown solid. LC/MS (ESI): 325.1, 327.1 [M+H]⁺.

Step 4: Methyl 3-(bromomethyl)-6-(1,3-dimethylpyrazol-4-yl)pyrazine-2-carboxylate (440 mg, 1.35 mmol, 1.0 eq) was suspended in 28% aqueous ammonium hydroxide and the mixture was stirred at RT for 8 h. The mixture was filtered, and the residue was washed with water and methanol. The solid was collected, dried under vacuum, and used in the next step without further purification. LC/MS (ESI): 230.1 [M+H]⁺

Step 5: To a round bottom flask charged with 3-(1,3-dimethylpyrazol-4-yl)-6,7-dihydro-5H-pyrrolo[4,3-b]pyrazin-5-one (40 mg, 0.17 mmol, 1.0 eq), tert-butyl (1R,3s,5S)-3-((6-bromopyridazin-3-yl)oxy)-1,5-dimethyl-8-azabicyclo[3.2.1]octane-8-carboxylate (71.95 mg, 0.17 mmol, 1.0 eq), Cs₂CO₃ (170.13 mg, 0.52 mmol, 3.0 eq), and XantPhos Pd G4 (16.79 mg, 0.02 mmol, 0.1 eq) was added 1,4-dioxane (4 mL). The reaction mixture was degassed with argon for 5 min and was then heated to 90° C. for 8 h. The reaction mixture was cooled to RT and diluted with EA. The organic phase was washed with brine, dried over sodium sulfate, and concentrated under vacuum. The crude product was purified by silica gel column chromatography with DCM/MeOH 20/1, v/v to obtain 2-methylpropan-2-yl (1R,3s,5S)-3-({6-[2-(1,3-dimethylpyrazol-4-yl)-7-oxo-6,7-dihydro-5H-pyrrolo[4,3-b]pyrazin-6-yl]-1,2-diazin-3-yl}oxy)-1,5-dimethyl-8-azabicyclo[3.2.1]octane-8-carboxylate (40.2 mg), which was dissolved HCl in dioxane (2 mL, 4.0 mol/L). The reaction was stirred at RT for 1 h, and was then concentrated. The resulting residue was purified by RP HPLC using MeCN and H₂O as eluent to provide 3-(1,3-dimethylpyrazol-4-yl)-6-(6-{[(1S,3s,5R)-1,5-dimethyl-8-azabicyclo[3.2.1]octan-3-yl]oxy}-1,2-diazin-3-yl)-6,7-dihydro-5H-pyrrolo[4,3-b]pyrazin-5-one (25 mg, 30%) as a white solid.

¹H NMR (400 MHz, DMSO-d₆) δ=9.10 (s, 1H), 8.49 (s, 2H), 8.30 (s, 1H), 7.35 (d, J=10.0 Hz, 1H), 5.16 (s, 2H), 5.02 (td, J=5.6, 11.3 Hz, 1H), 3.85 (s, 3H), 2.91 (s, 3H), 2.52 (br s, 3H), 1.98 (br d, J=7.6 Hz, 2H), 1.79 (br t, J=10.9 Hz, 2H), 1.74-1.68 (m, 2H), 1.66 (br d, J=5.1 Hz, 2H), 1.31 (s, 6H); LC/MS (ESI): 474.3 [M+H]⁺.

### Example 33. Synthesis of 3-(2,5-dimethyl-2H-1,2,3-triazol-4-yl)-6-(6-(((1R,3s,5S)-1,5-dimethyl-8-azabicyclo[3.2.1]octan-3-yl)oxy)pyridazin-3-yl)-6,7-dihydro-5H-pyrrolo[3,4-b]pyrazin-5-one (Compound No. 453) PGP-155 C₃

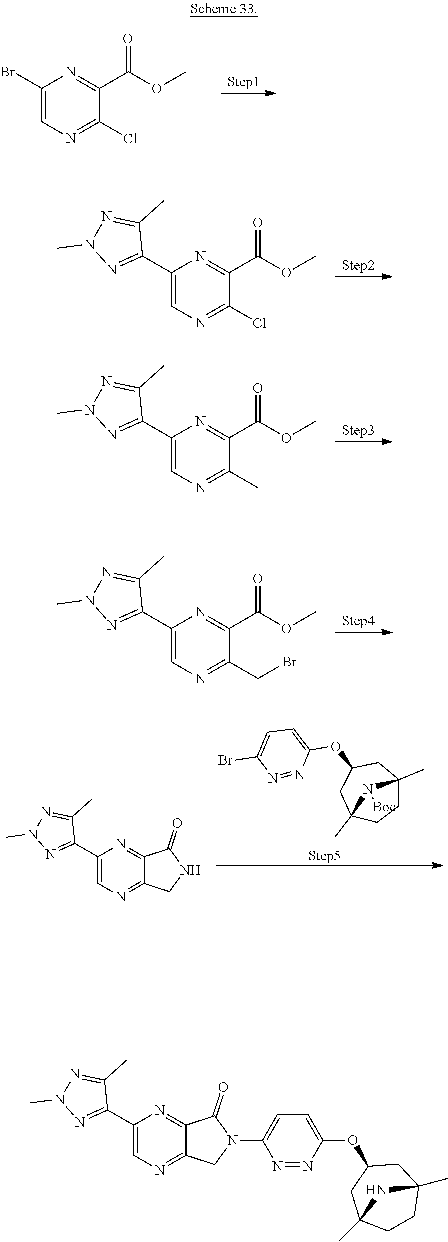

Step 1: 4-Bromo-2,5-dimethyl-2H-1,2,3-triazole (262.0 mg, 1.5 mmol, 1.5 eq), 4,4,4′,4′,5,5,5′,5′-octamethyl-2,2′-bi(1,3,2-dioxaborolane (762 mg, 3.0 mmol, 3.0 eq), potassium acetate (300 mg, 3.0 mmol, 3.0 eq) and PdCl₂(dppf)·CH₂Cl₂ adduct (109.3 mg, 0.15 mmol, 0.1 eq) were charged to a three-neck 100 mL round bottom flask under nitrogen and anhydrous dioxane (9 mL) was added and the reaction mixture was heated to 90° C. When full conversion was achieved, the reaction mixture was cooled to room temperature. Then methyl 6-bromo-3-chloropyrazine-2-carboxylate (249.9 mg, 1.0 mmol, 1.0 eq), aqueous potassium carbonate (2 M, 2 mL) and PdCl₂(dppf)·CH₂Cl₂ adduct (109.3 mg, 0.15 mmol, 0.1 eq) were added and the mixture was heated to 90° C. When full conversion was achieved, the mixture was cooled, filtered through a pad of Celite, and the filter cake was rinsed with EA (100 mL). The filtrate was washed with brine and the organic layer was collected, dried over sodium sulfate, and concentrated in vacuo. The crude product was purified by silica gel column chromatography with DCM/MeOH, 20/1, v/v to obtain methyl 3-chloro-6-(2,5-dimethyl-2H-1,2,3-triazol-4-yl)pyrazine-2-carboxylate (165.5 mg, 62%) as a brown solid. LC/MS (ESI): 268.1 [M+H]⁺.

Step 2: Methyl 3-chloro-6-(2,5-dimethyl-2H-1,2,3-triazol-4-yl)pyrazine-2-carboxylate (500 mg, 1.87 mmol, 1.2 eq), 2,4,6-trimethyl-1,3,5,2,4,6-trioxatriborinane (1.29 mL, 2.25 mmol, 1.0 eq), potassium carbonate (777.31 mg, 5.62 mmol, 3.0 eq) and PdCl₂(dppf)·CH₂Cl₂ adduct (137.05 mg, 0.19 mmol, 0.1 eq) were charged to a three-neck 100 mL round bottom flask under nitrogen. Anhydrous dioxane (10 mL) was added and the reaction mixture was heated to 110° C. 3 h. Then the mixture was cooled, filtered through a pad of Celite, and the filter cake was rinsed with EA (100 mL). The filtrate was washed with brine and the organic layer was collected, dried over sodium sulfate, and concentrated in vacuo. The crude product was purified by silica gel column chromatography with PE/EA 5/1, v/v to afford methyl 6-(2,5-dimethyl-2H-1,2,3-triazol-4-yl)-3-methylpyrazine-2-carboxylate (430 mg, 93.13%).

LC/MS (ESI): 247.1 [M+H]⁺.

Step 3: Methyl 6-(2,5-dimethyl-2H-1,2,3-triazol-4-yl)-3-methylpyrazine-2-carboxylate (430 mg, 1.75 mmol, 1.0 eq), N-bromosuccinimide (371.67 mg, 2.10 mmol, 1.2 eq) and benzoyl peroxide (129.17 mg, 0.79 mmol, 0.45 eq) were combined with carbon tetrachloride (10 mL) and the reaction mixture was heated to 80° C. and stirred for 16 h. The reaction mixture was cooled to RT and concentrated under vacuum. The resulting residue was purified by silica gel column chromatography with PE/EA to afford methyl 3-(bromomethyl)-6-(2,5-dimethyl-2H-1,2,3-triazol-4-yl)pyrazine-2-carboxylate (440 mg, 77.32%) as a brown solid. LC/MS (ESI): 325.1, 327.1 [M+H]⁺.

Step 4: Methyl 3-(bromomethyl)-6-(2,5-dimethyl-2H-1,2,3-triazol-4-yl)pyrazine-2-carboxylate (440 mg, 1.35 mmol, 1.0 eq) was suspended in 28% aqueous ammonium hydroxide and the mixture was stirred at RT for 8 h. Then the mixture was filtered, and the residue was washed with water and methanol. The solid was collected, dried under vacuum, and used in the next step without further purification.

LC/MS (ESI): 230.1 [M+H]⁺

Step 5: To a round bottom flask charged with 3-(2,5-dimethyl-2H-1,2,3-triazol-4-yl)-6,7-dihydro-5H-pyrrolo[3,4-b]pyrazin-5-one (40 mg, 0.17 mmol, 1.0 eq), tert-butyl (1R,3s,5S)-3-((6-bromopyridazin-3-yl)oxy)-1,5-dimethyl-8-azabicyclo[3.2.1]octane-8-carboxylate (71.95 mg, 0.17 mmol, 1.0 eq), Cs₂CO₃ (170.13 mg, 0.52 mmol, 3.0 eq), and XantPhos Pd G4 (16.79 mg, 0.02 mmol, 0.1 eq) was added 1,4-dioxane (4 mL). The reaction mixture was degassed with argon for 5 min and was then heated at 90° C. for 8 h. The reaction mixture was cooled to RT and diluted with EA. The organic phase was washed with brine, dried over sodium sulfate, and concentrated under vacuum. The crude product was purified by silica gel column chromatography with DCM/MeOH 20/1, v/v, and was then dissolved in HCl in dioxane (2 mL, 4.0 mol/L). The reaction was stirred at RT for 1 h and was then concentrated. The resulting residue was purified by RP HPLC using MeCN and H₂O as eluent to provide 3-(1,3-dimethyl-1H-pyrazol-4-yl)-6-(6-(((1R,3s,5S)-1,5-dimethyl-8-azabicyclo[3.2.1]octan-3-yl)oxy)pyridazin-3-yl)-6,7-dihydro-5H-pyrrolo[3,4-b]pyrazin-5-one (25 mg, 30%) as a white solid.

¹H NMR (400 MHz, DMSO-d₆) δ=9.34 (s, 1H), 8.71 (d, J=9.6 Hz, 1H), 7.35 (d, J=9.6 Hz, 1H), 5.52-5.42 (m, 1H), 5.25 (s, 2H), 4.23 (s, 3H), 2.62 (s, 3H), 2.17 (br dd, J=5.8, 11.9 Hz, 2H), 1.80 (br d, J=6.6 Hz, 2H), 1.53 (br d, J=6.9 Hz, 2H), 1.46-1.37 (m, 2H), 1.21 (s, 6H); LC/MS (ESI): 462.2 [M+H]⁺.

In some embodiments of the disclosure, the compounds can be made by similar means according to Examples 1-33. Table 7 provides for physical data for the compounds disclosed herein.

TABLE 7

| No. | NMR/MS |
|---|---|
| 1 | LC/MS (ESI): 405.2 [M + H]⁺ |
| 2 | LC/MS (ESI): 448.2 [M + H]⁺ |
| 3 | LC/MS (ESI): 461.2 [M + H]⁺ |
| 4 | LC/MS (ESI): 402.20 [M + H]⁺ |
| 5 | LC/MS (ESI): 390.2 [M + H]⁺ |
| 6 | LC/MS (ESI): 448.2 [M + H]⁺ |
| 7 | LC/MS (ESI): 462.2 [M + H]⁺ |
| 8 | LC/MS (ESI): 506.3 [M + H]⁺ |
| 9 | LC/MS (ESI): 498.2 [M + H]⁺ |
| 10 | LC/MS (ESI): 488.2 [M + H]⁺ |
| 11 | LC/MS (ESI): 386.17 [M + H]⁺ |
| 12 | LC/MS (ESI): 376.1 [M + H]⁺ |
| 13 | LC/MS (ESI): 418.1 [M + H]⁺ |
| 14 | LC/MS (ESI): 388.18 [M + H]⁺ |
| 15 | LC/MS (ESI): 434.3 [M + H]⁺ |
| 16 | LC/MS (ESI): 428.18 [M + H]⁺ |
| 17 | ¹H NMR (C₂DF₃O₂, 400 MHz) δ 9.94 (d, J = 9.5 Hz, 1H), 9.38 (s, 1H), 9.08 (s, |
| | 1H), 9.00 (s, 1H), 8.90 (s, 1H), 8.80 (d, J = 9.5 Hz, 1H), 6.92-6.49 (m, 1H), |
| | 5.73 (s, 2H), 4.41 (s, 3H), 3.57 (br d, J = 7.0 Hz, 2H), 2.95 (br d, J = 3.4 Hz, |
| | 1H), 2.10-2.03 (m, 2H), 2.01-1.91 (m, 2H), 1.73 (s, 6H), 1.67 (s, 6H); LC/MS |
| | (ESI): 446.3 [M + H]⁺ |
| 18 | LC/MS (ESI): 476.27 [M + H]⁺ |
| 19 | ¹H NMR (DMSO-d₆, 400 MHz) δ 8.92 (s, 1H), 8.45-8.40 (m, 2H), 8.13 (s, |
| | 1H), 7.99 (s, 1H), 7.26 (d, J = 10.0 Hz, 1H), 5.19 (s, 2H), 4.87-4.76 (m, 1H), |
| | 3.90 (s, 3H), 3.58-3.51 (m, 2H), 2.86 (s, 3H), 2.10 (br d, J = 12.9 Hz, 2H), |
| | 1.79-1.68 (m, 2H), 1.62 (br d, J = 7.0 Hz, 2H), 1.33 (br t, J = 12.2 Hz, 2H), 1H |
| | not observed (NH); LC/MS (ESI): 431.2 [M + H]⁺ |
| 20 | ¹H NMR (DMSO-d₆, 400 MHz) δ 8.93 (s, 1H), 8.47 (d, J = 9.9 Hz, 1H), 8.44 |
| | (s, 1H), 8.14 (s, 1H), 8.01 (s, 1H), 7.33 (d, J = 10.0 Hz, 1H), 5.21 (s, 2H), 5.07- |
| | 4.94 (m, 1H), 3.90 (s, 3H), 2.91 (s, 3H), 2.05-1.92 (m, 2H), 1.68 (br s, 6H), |
| | 1.31 (br s, 6H), 1H not observed (NH); LC/MS (ESI): 459.3 [M + H]⁺ |
| 21 | LC/MS (ESI): 506.3 [M + H]⁺ |
| 22 | LC/MS (ESI): 417.2 [M + H]⁺ |
| 23 | LC/MS (ESI): 490.3 [M + H]⁺ |
| 24 | LC/MS (ESI): 474.2 [M + H]⁺ |
| 25 | LC/MS (ESI): 430.2 [M + H]⁺ |
| 26 | LC/MS (ESI): 492.2 [M + H]⁺ |
| 27 | LC/MS (ESI): 429.2 [M + H]⁺ |
| 28 | ¹H NMR (DMSO-d₆, 400 MHz) δ 9.02 (d, J = 6.3 Hz, 2H), 8.71 (d, J = 9.6 |
| | Hz, 1H), 8.52 (s, 1H), 8.28 (s, 1H), 8.04-7.72 (m, 1H), 7.34 (d, J = 9.5 Hz, |
| | 1H), 5.63 (br s, 1H), 5.28 (s, 2H), 2.28-2.07 (m, 2H), 1.58-1.34 (m, 2H), 1.27 |
| | (br s, 6H), 1.22-1.03 (m, 6H), 1H not observed (NH); LC/MS (ESI): 484.2 |
| | [M + H]⁺ |
| 29 | LC/MS (ESI): 484.3 [M + H]⁺ |
| 30 | LC/MS (ESI): 516.2 [M + H]⁺ |
| 31 | LC/MS (ESI): 490.2 [M + H]⁺ |
| 32 | LC/MS (ESI): 445.2 [M + H]⁺ |
| 33 | ¹H NMR (400 MHZ, DMSO-d₆) δ 9.58 (s, 2H), 9.30 (s, 1H), 9.21 (s, 1H), 9.17 |
| | (br s, 1H), 8.65-8.51 (m, 2H), 7.67-7.52 (m, 1H), 5.32 (s, 2H), 3.93-3.89 (m, |
| | 2H), 3.79 (s, 2H), 3.40 (br s, 2H), 1.08-1.00 (m, 2H), 1.00-0.91 (m, 2H); |
| | LC/MS (ESI): 400.3 [M + H]⁺ |
| 34 | LC/MS (ESI): 433.2 [M + H]⁺ |
| 35 | LC/MS (ESI): 497.2 [M + H]⁺ |
| 36 | LC/MS (ESI): 417.2 [M + H]⁺ |
| 37 | LC/MS (ESI): 453.4 [M + H]⁺ |
| 38 | LC/MS (ESI): 403.2 [M + H]⁺ |
| 39 | LC/MS (ESI): 417.2 [M + H]⁺ |
| 40 | LC/MS (ESI): 431.3 [M + H]⁺ |
| 41 | LC/MS (ESI): 419.2 [M + H]⁺ |
| 42 | LC/MS (ESI): 434.2 [M + H]⁺ |
| 43 | LC/MS (ESI): 475.3 [M + H]⁺ |
| 44 | LC/MS (ESI): 403.2 [M + H]⁺ |
| 45 | LC/MS (ESI): 422.3 [M + H]⁺ |
| 46 | LC/MS (ESI): 419.5 [M + H]⁺ |
| 47 | LC/MS (ESI): 475.4 [M + H]⁺ |
| 48 | LC/MS (ESI): 431.3 [M + H]⁺ |
| 49 | LC/MS (ESI): 464.3 [M + H]⁺ |
| 50 | LC/MS (ESI): 448.2 [M + H]⁺ |
| 51 | ¹H NMR (400 MHZ, DMSO-d₆) δ = 8.97-8.92 (m, 1H), 8.85-8.75 (m, 1H), |
| | 8.55-8.49 (m, 1H), 8.38-8.32 (m, 1H), 7.98-7.89 (m, 1H), 7.86-7.81 (m, |
| | 1H), 7.48-7.40 (m, 1H), 5.22-5.17 (m, 2H), 5.13-5.02 (m, 1H), 3.84-3.78 |
| | (m, 3H), 2.96-2.91 (m, 3H), 2.47 (s, 3H), 1.97-1.87 (m, 2H), 1.82-1.75 (m, |
| | 2H), 1.54-1.49 (m, 6H), 1.45-1.39 (m, 6H); LC/MS (ESI): 475.3 [M + H]⁺ |
| 52 | LC/MS (ESI): 417.2 [M + H]⁺ |
| 53 | LC/MS (ESI): 431.3 [M + H]⁺ |
| 54 | LC/MS (ESI): 476.3 [M + H]⁺ |
| 55 | ¹H NMR (DMSO-d₆, 400MHz) δ = 9.33 (s, 1H), 8.97 (s, 1H), 8.37 (s, 1H), |
| | 7.88 (s, 1H), 5.17 (s, 2H), 5.07 (m, 1H), 3.81 (s, 3H), 3.03 (s, 3H), 2.48 (s, |
| | 3H), 1.81 (br d, J = 7.4 Hz, 2H), 1.54-1.69 (m, 6H), 1.23 (s, 6H), 1H not |
| | observed (NH); LC/MS (ESI): 474.3 [M + H]⁺ |
| 56 | ¹H NMR (400 MHZ, DMSO-d₆) δ = 9.06 (s, 1H), 8.72 (s, 1H), 8.71 (d, 2H, J = |
| | 9.0 Hz), 8.2-8.3 (m, 1H), 7.99 (s, 1H), 7.35 (dd, 1H, J = 1.4, 9.6 Hz), 5.6-5.7 |
| | (m, 1H), 5.29 (s, 2H), 4.28 (d, 2H, J = 7.3 Hz), 2.13 (br dd, 2H, J = 3.9, 15.8 |
| | Hz), 1.48 (t, 3H, J = 7.3 Hz), 1.36 (br d, 2H, J = 11.9 Hz), 1.3-1.3 (s, 6H), 1.17 |
| | (s, 6H); 1H not observed (NH); LC/MS (ESI): 458.2 [M + H]⁺ |
| 57 | LC/MS (ESI): 492.2 [M + H]⁺ |
| 58 | LC/MS (ESI): 515.2 [M + H]⁺ |
| 59 | ¹H NMR (DMSO-d₆, 400MHz) δ = 8.98 (s, 1H), 8.69 (d, J = 9.6 Hz, 1H), 8.37 |
| | (s, 1H), 8.19 (br s, 1H), 7.88 (s, 1H), 7.32 (d, J = 9.6 Hz, 1H), 5.63-5.53 (m, |
| | 1H), 5.24 (s, 2H), 3.81 (s, 3H), 2.48 (s, 3H), 2.11 (br dd, J = 3.6, 12.3 Hz, 1H), |
| | 1.88-1.80 (m, 1H), 1.63 (br dd, J = 4.3, 11.8 Hz, 1H), 1.37 (t, J = 11.6 Hz, |
| | 1H), 1.21 (s, 3H), 1.10 (s, 3H), 0.59-0.52 (m, 2H), 0.51-0.44 (m, 1H), 0.40- |
| | 0.33 (m, 1H); LC/MS (ESI): 460.1 [M + H]⁺ |
| 60 | LC/MS (ESI): 460.2 [M + H]⁺ |
| 61 | LC/MS (ESI): 527.2 [M + H]⁺ |
| 62 | 'H NMR (DMSO-d₆, 400 MHz) δ = 9.03 (s, 1H), 8.46 (d, J = 9.8 Hz, 1H), |
| | 8.31 (s, 1H), 7.89 (s, 1H), 7.34 (d, J = 9.9 Hz, 1H), 5.25 (s, 2H), 5.05-4.93 |
| | (m, 1H), 2.91 (s, 3H), 2.64 (s, 6H), 1.96 (br d, J = 7.8 Hz, 2H), 1.80-1.72 (m, |
| | 2H), 1.68 (br d, J = 7.5 Hz, 2H), 1.63 (br dd, J = 5.4, 12.9 Hz, 2H), 1.30 (s, |
| | 6H); LC/MS (ESI): 490.2 [M + H]⁺ |
| 63 | LC/MS (ESI): 516.2 [M + H]⁺ |
| 64 | LC/MS (ESI): 462.3 [M + H]⁺ |
| 65 | LC/MS (ESI): 530.2 [M + H]⁺ |
| 66 | LC/MS (ESI): 543.2 [M + H]⁺ |
| 67 | LC/MS (ESI): 474.2 [M + H]⁺ |
| 68 | LC/MS (ESI): 447.2 [M + H]⁺ |
| 69 | ¹H NMR (DMSO-d₆, 400 MHZ) δ = 9.06 (s, 1H), 8.69 (d, J = 9.6 Hz, 1H), 8.24 |
| | (s, 1H), 7.92 (s, 1H), 7.35 (d, J = 9.6 Hz, 1H), 5.49 (s, 1H), 5.28 (s, 2H), 2.65 |
| | (s, 6H), 2.22 (br dd, J = 5.9, 12.3 Hz, 2H), 1.88 (br d, J = 7.4 Hz, 2H), 1.60 (br |
| | d, J = 6.9 Hz, 2H), 1.49 (br t, J = 11.4 Hz, 2H), 1.25 (s, 6H); |
| | LC/MS (ESI): 477.2 [M + H]⁺ |
| 70 | LC/MS (ESI): 460.2 [M + H]⁺ |
| 71 | ¹H NMR (DMSO-d₆, 400MHz) δ = 9.03 (s, 1H), 8.63 (s, 1H), 8.47 (d, J = 9.9 |
| | Hz, 1H), 8.29 (s, 1H), 7.92 (s, 1H), 7.33 (d, J = 10.0 Hz, 1H), 5.27 (s, 2H), |
| | 5.10-5.00 (m, 1H), 3.99 (s, 3H), 2.91 (s, 3H), 1.67-1.58 (m, 4H), 1.35 (s, |
| | 6H), 1.22 (s, 6H); LC/MS (ESI): 529.2 [M + H]⁺ |
| 72 | ¹H NMR (DMSO-d₆, 400 MHZ) δ = 8.98 (s, 1H), 8.71 (d, J = 9.6 Hz, 1H), 8.37 |
| | (s, 1H), 8.23 (s, 1H), 7.88 (s, 1H), 7.37 (d, J = 9.6 Hz, 1H), 5.51 (br t, J = 5.7 |
| | Hz, 1H), 5.23 (s, 2H), 3.81 (s, 3H), 2.47-2.48 (s, 3H), 2.36 (br dd, J = 13.0, 5.9 |
| | Hz, 2H), 2.06 (br d, J = 7.8 Hz, 2H), 1.79 (br d, J = 8.0 Hz, 2H), 1.69 (br t, J = |
| | 11.9 Hz, 2H), 1.38 (s, 6H); LC/MS (ESI): 460.2 [M + H]⁺ |
| 73 | LC/MS (ESI): 461.2 [M + H]⁺ |
| 74 | ¹H NMR (DMSO-d₆, 400MHz) d: 9.28 (br d, J = 9.8 Hz, 1H), 9.13 (br d, J = |
| | 9.0 Hz, 1H), 9.06 (s, 1H), 8.73 (d, J = 9.6 Hz, 1H), 8.64 (s, 1H), 7.96 (s, 1H), |
| | 7.40 (d, J = 9.6 Hz, 1H), 5.53 (td, J = 5.2, 10.9 Hz, 1H), 5.27 (s, 2H), 3.99 (s, |
| | 3H), 2.43 (br dd, J = 5.9, 13.1 Hz, 2H), 2.15 (br d, J = 8.1 Hz, 2H), 1.95-1.82 |
| | (m, 4H), 1.47 (s, 6H); LC/MS (ESI): 514.2 [M + H]⁺ |
| 75 | LC/MS (ESI): 498.2 [M + H]⁺ |
| 76 | ¹H NMR (DMSO-d₆, 400MHz) δ = 8.98 (s, 1H), 8.72 (d, J = 9.5 Hz, 1H), 8.38 |
| | (s, 1H), 7.89 (s, 1H), 7.38 (br d, J = 9.6 Hz, 1H), 5.74-5.58 (m, 1H), 5.25 (s, |
| | 2H), 3.82 (s, 3H), 2.48 (s, 3H), 2.30-2.13 (m, 2H), 1.53 (br d, J = 6.5 Hz, |
| | 2H), 1.45-1.16 (m, 12H), 1H not observed (NH); LC/MS (ESI): 462.3 |
| | [M + H]⁺ |
| 77 | 'H NMR (DMSO-d₆, 400 MHZ) δ = 9.07 (s, 1H), 8.94 (s, 1H), 8.71 (d, J = 9.6 |
| | Hz, 1H), 8.36-8.25 (m, 1H), 8.12 (s, 1H), 8.01-7.62 (m, 1H), 7.35 (d, J = 9.5 |
| | Hz, 1H), 5.57-5.45 (m, 1H), 5.28 (s, 2H), 2.57 (s, 3H), 2.22 (dd, J = 5.9, 12.3 |
| | Hz, 2H), 1.88 (br d, J = 7.5 Hz, 2H), 1.62 (br d, J = 7.6 Hz, 2H), 1.56-1.48 |
| | (m, 2H), 1.27 (s, 6H); LC/MS (ESI): 496.2 [M + H]⁺ |
| 78 | LC/MS (ESI): 498.2 [M + H]⁺ |
| 79 | ¹H NMR (DMSO-d₆, 400MHz) δ: 8.87-8.97 (m, 1H), 8.70 (d, J = 9.6 Hz, 1H), |
| | 8.31 (s, 1H), 7.83 (d, J = 0.9 Hz, 1H), 7.41 (d, J = 9.6 Hz, 1H), 5.43-5.62 (m, |
| | 1H), 5.15 (s, 2H), 4.91-5.12 (m, 1H), 3.75 (s, 3H), 2.41 (s, 3H), 2.17-2.31 (m, |
| | 1H), 1.92-2.14 (m, 2H), 1.64-1.91 (m, 3H), 1.25-1.50 (m, 6H), 1H not |
| | observed (NH); LC/MS (ESI): 478.2 [M + H]⁺ |
| 80 | LC/MS (ESI): 463.2 [M + H]⁺ |
| 81 | LC/MS (ESI): 434.2 [M + H]⁺ |
| 82 | LC/MS (ESI): 496.2 [M + H]⁺ |
| 83 | LC/MS (ESI): 461.2 [M + H]⁺ |
| 84 | LC/MS (ESI): 463.3 [M + H]⁺ |
| 85 | ¹HNMR (DMSO-d₆, 400MHz) δ = 8.97 (s, 1H), 8.48 (d, J = 9.9 Hz, 1H), 8.30 |
| | (s, 1H), 7.85 (s, 1H), 7.35 (d, J = 10.0 Hz, 1H), 5.22 (s, 2H), 5.08 (td, J = 5.6, |
| | 11.6 Hz, 1H), 3.83 (s, 3H), 2.92 (s, 3H), 2.48 (s, 3H), 2.13 (br d, J = 8.1 Hz, |
| | 2H), 2.01-1.93 (m, 2H), 1.92-1.86 (m, 2H), 1.79 (br dd, J = 5.4, 13.4 Hz, |
| | 2H), 1.42 (s, 6H), 1H not observed (NH); LC/MS (ESI): 473.3 [M + H]⁺ |
| 86 | LC/MS (ESI): 479.2 [M + H]⁺ |
| 88 | LC/MS (ESI): 477.1 [M + H]⁺ |
| 89 | LC/MS (ESI): 473.6 [M + H]⁺ |
| 90 | ¹H NMR (DMSO-d₆, 400 MHZ) δ = 8.47-8.41 (m, 2H), 8.35 (s, 1H), 7.89 (s, |
| | 1H), 7.32 (d, J = 10.0 Hz, 1H), 5.15 (s, 2H), 5.07-4.93 (m, 1H), 3.82 (s, 3H), |
| | 2.90 (s, 3H), 2.48 (s, 3H), 1.95-1.89 (m, 2H), 1.77-1.58 (m, 6H), 1.28 (s, |
| | 6H); LC/MS (ESI): 507.2 [M + H]⁺ |
| 91 | LC/MS (ESI): 466.2 [M + H]⁺ |
| 92 | LC/MS (ESI): 476.3 [M + H]⁺ |
| 93 | ¹H NMR (DMSO-d₆, 400 MHz) δ , 8.96 (br d, J = 8.8 Hz, 1H), 8.85 (br d, J = |
| | 9.3 Hz, 1H), 8.70 (d, J = 9.6 Hz, 1H), 8.47 (s, 1H), 7.94 (s, 1H), 7.41 (d, J = |
| | 9.6 Hz, 1H), 5.61-5.47 (m, 1H), 5.16 (s, 2H), 3.82 (s, 3H), 2.48-2.42 (m, |
| | 5H), 2.16 (br d, J = 8.0 Hz, 2H), 1.95-1.75 (m, 4H), 1.45 (s, 6H); LC/MS |
| | (ESI): 494.1 [M + H]⁺ |
| 94 | LC/MS (ESI): 474.2 [M + H]⁺ |
| 95 | LC/MS (ESI): 418.2 [M + H]⁺ |
| 96 | ¹H NMR (METHANOL-d₄, 400 MHZ) δ: 9.02 (s, 1H), 8.85 (d, J = 9.6 Hz, |
| | 1H), 8.27 (s, 1H), 7.26 (d, J = 9.6 Hz, 1H), 5.69-5.57 (m, 1H), 5.29 (s, 2H), |
| | 4.19 (s, 3H), 2.61 (s, 3H), 2.61-2.56 (m, 2H), 2.38-2.29 (m, 2H), 2.08-2.00 |
| | (m, 2H), 1.86 (dd, J = 11.4, 13.2 Hz, 2H), 1.55 (s, 6H), 'H not observed (NH); |
| | LC/MS (ESI): 461.2 [M + H]⁺ |
| 97 | LC/MS (ESI): 461.3 [M + H]⁺ |
| 98 | ¹H NMR (DMSO-d₆, 400 MHZ) δ 8.98 (s, 1H), 8.70 (d, J = 9.6 Hz, 1H), 8.37 |
| | (s, 1H), 8.28 (s, 1H), 7.88 (s, 1H), 7.34 (d, J = 9.6 Hz, 1H), 5.51 (s, 1H), 5.24 |
| | (s, 2H), 3.81 (s, 3H), 2.48 (s, 3H), 2.26 (br dd, J = 5.6, 12.4 Hz, 2H), 1.90 (br t, |
| | J = 7.1 Hz, 2H), 1.71-1.48 (m, 6H), 1.30 (s, 3H), 0.91 (t, J = 7.4 Hz, 3H); |
| | LC/MS (ESI): 474.2 [M + H]⁺ |
| 99 | ¹H NMR (DMSO-d₆, 400 MHz) δ = 8.96 (s, 1H), 8.71 (d, J = 9.5 Hz, 1H), 8.45 |
| | (s, 1H), 8.27 (s, 1H), 8.15 (s, 1H), 8.05 (s, 1H), 7.35 (d, J = 9.6 Hz, 1H), 5.48 |
| | (br d, J = 6.0 Hz, 1H), 5.24 (s, 2H), 3.90 (s, 3H), 2.25 (br dd, J = 6.1, 12.2 Hz, |
| | 2H), 1.92 (br d, J = 7.5 Hz, 2H), 1.66 (br d, J = 7.5 Hz, 2H), 1.57 (br t, J = |
| | 11.6 Hz, 2H), 1.30 (s, 6H); LC/MS (ESI): 446.2 [M + H]⁺ |
| 100 | ¹H NMR (METHANOL-d₄, 400 MHz) d: 8.97 (s, 1H), 8.88 (d, J = 9.6 Hz, |
| | 1H), 8.13 (s, 1H), 7.95 (s, 1H), 7.28 (d, J = 9.6 Hz, 1H), 5.71-5.58 (m, 1H), |
| | 5.30 (s, 2H), 3.90 (s, 3H), 2.66 (dd, J = 5.9, 13.9 Hz, 2H), 2.54 (s, 3H), 2.39- |
| | 2.30 (m, 2H), 1.99 (br d, J = 8.5 Hz, 2H), 1.92-1.80 (m, 6H), 1.08 (t, J = 7.5 |
| | Hz, 6H), 1H not observed (NH); LC/MS (ESI): 488.3 [M + H]⁺ |
| 101 | LC/MS (ESI): 486.2 [M + H]⁺ |
| 102 | LC/MS (ESI): 474.2 [M + H]⁺ |
| 103 | LC/MS (ESI): 542.4 [M + H]⁺ |
| 104 | LC/MS (ESI): 486.2 [M + H]⁺ |
| 105 | LC/MS (ESI): 511.1 [M + H]⁺ |
| 106 | LC/MS (ESI): 481.1 [M + H]⁺ |
| 107 | LC/MS (ESI): 464.2 [M + H]⁺ |
| 108 | LC/MS (ESI): 478.1 [M + H]⁺ |
| 109 | LC/MS (ESI): 490.3 [M + H]⁺ |
| 110 | LC/MS (ESI): 495.1 [M + H]⁺ |
| 111 | ¹HNMR (DMSO-d₆, 400 MHZ) δ = 9.32 (s, 1H), 8.94 (br s, 1H), 8.86 (br s, |
| | 1H), 8.74 (dd, J = 9.6, 0.9 Hz, 1H), 7.43 (d, J = 9.5 Hz, 1H), 5.49-5.60 (m, |
| | 1H), 5.21 (s, 2H), 2.84 (s, 3H), 2.67 (s, 3H), 2.42-2.48 (m, 2H), 2.17 (br d, J = |
| | 8.1 Hz, 2H), 1.89 (br d, J = 8.3 Hz, 2H), 1.76-1.85 (m, 2H), 1.46 (s, 6H); |
| | LC/MS (ESI): 478.3 [M + H]⁺ |
| 112 | ¹H NMR (DMSO-d₆, 400 MHZ) δ = 9.15 (br s, 1H), 9.00 (s, 1H), 8.74 (d, J = |
| | 9.6 Hz, 1H), 8.58 (s, 1H), 8.40 (s, 1H), 7.40 (d, J = 9.6 Hz, 1H), 5.58-5.48 |
| | (m, 1H), 5.26 (s, 2H), 2.72-2.68 (m, 3H), 2.48-2.38 (m, 2H), 2.21-2.08 (m, |
| | 2H), 1.94-1.77 (m, 4H), 1.46 (s, 6H); LC/MS (ESI): 463.1 [M + H]⁺ |
| 113 | LC/MS (ESI): 460.2 [M + H]⁺ |
| 114 | LC/MS (ESI): 463.1 [M + H]⁺ |
| 115 | ¹H NMR (DMSO-d₆, 400 MHZ) δ = 8.97 (s, 1H), 8.70 (d, J = 9.6 Hz, 1H), 8.37 |
| | (s, 1H), 8.25 (s, 1H), 7.88 (d, J = 1.0 Hz, 1H), 7.35 (d, J = 9.6 Hz, 1H), 5.51 |
| | (ddd, J = 4.4, 6.8, 10.9 Hz, 1H), 5.25 (s, 2H), 3.81 (s, 3H), 2.55-2.51 (m, 1H), |
| | 2.48 (s, 3H), 2.21-2.12 (m, 1H), 2.06 (q, J = 10.0 Hz, 3H), 1.91-1.83 (m, |
| | 1H), 1.79-1.63 (m, 2H), 1.51-1.37 (m, 2H), 1.19 (s, 3H), 1.13 (s, 3H); |
| | LC/MS (ESI): 474.2 [M + H]⁺ |
| 116 | LC/MS (ESI): 478.2 [M + H]⁺ |
| 117 | LC/MS (ESI): 461.3 [M + H]⁺ |
| 120 | LC/MS (ESI): 405.1 [M + H]⁺ |
| 123 | LC/MS (ESI): 417.3 [M + H]⁺ |
| 124 | LC/MS (ESI): 433.3 [M + H]⁺ |
| 134 | LC/MS (ESI): 445.3 [M + H]⁺ |
| 135 | LC/MS (ESI): 462.3 [M + H]⁺ |
| 136 | LC/MS (ESI): 376.2 [M + H]⁺ |
| 137 | LC/MS (ESI): 390.2 [M + H]⁺ |
| 138 | LC/MS (ESI): 476.2 [M + H]⁺ |
| 139 | LC/MS (ESI): 488.3 [M + H]⁺ |
| 140 | LC/MS (ESI): 484.2 [M + H]⁺ |
| 141 | ¹H NMR (DMSO-d₆, 400 MHz) δ 8.95 (d, J = 2.0 Hz, 1H), 8.70 (d, J = 9.6 Hz, |
| | 1H), 8.22-8.13 (m, 2H), 7.32 (d, J = 9.5 Hz, 1H), 5.62 (ddd, J = 4.1, 7.1, 11.1 |
| | Hz, 1H), 5.20 (s, 2H), 3.82 (s, 3H), 2.36 (s, 3H), 2.09 (br d, J = 9.9 Hz, 2H), |
| | 1.39-1.06 (m, 14H), 1H not observed (NH); LC/MS (ESI): 462.2 [M + H]⁺ |
| 142 | LC/MS (ESI): 498.3 [M + H]⁺ |
| 143 | LC/MS (ESI): 434.3 [M + H]⁺ |
| 144 | LC/MS (ESI): 448.2 [M + H]⁺ |
| 145 | LC/MS (ESI): 402.20 [M + H]⁺ |
| 146 | LC/MS (ESI): 388.18 [M + H]⁺ |
| 147 | LC/MS (ESI): 386.17 [M + H]⁺ |
| 148 | ¹H NMR (DMSO-d₆, 400 MHz) δ = 9.54 (br d, J = 10.6 Hz, 1H), 9.17 (d, J = |
| | 1.9 Hz, 1H), 8.81 (d, J = 10.3 Hz, 1H), 8.68-8.57 (m, 1H), 8.47 (s, 1H), 8.43 |
| | (d, J = 2.0 Hz, 1H), 8.17-8.14 (m, 1H), 8.05 (br s, 1H), 5.09 (s, 2H), 4.85- |
| | 4.77 (m, 1H), 3.90 (s, 3H), 3.08 (s, 3H), 2.21 (br t, J = 12.8 Hz, 2H), 1.81- |
| | 1.72 (m, 2H), 1.56 (s, 6H), 1.53 (s, 6H); LC/MS (ESI): 461.2 [M + H] + |
| 149 | ¹H NMR (DMSO-d₆, 400 MHZ) δ = 9.17 (d, J = 1.9 Hz, 1H), 8.49 (d, J = 10.0 |
| | Hz, 1H), 8.42 (d, J = 2.0 Hz, 1H), 8.40-8.31 (m, 3H), 7.32 (d, J = 10.0 Hz, |
| | 1H), 5.15 (s, 2H), 5.12-5.03 (m, 1H), 2.91 (s, 3H), 1.73 (br t, J = 12.4 Hz, |
| | 2H), 1.61 (dd, J = 3.1, 12.4 Hz, 2H), 1.39 (s, 6H), 1.28 (s, 6H), 1H not |
| | observed (NH); LC/MS (ESI): 447.3 [M + H]⁺ |
| 150 | ¹HNMR (DMSO-d₆, 400 MHz) δ 8.95 (d, J = 2.1 Hz, 1H), 8.70 (d, J = 9.6 Hz, |
| | 1H), 8.24 (d, J = 2.0 Hz, 1H), 7.34 (d, J = 9.6 Hz, 1H), 5.69-5.58 (m, 1H), |
| | 5.24 (s, 2H), 2.68 (s, 3H), 2.43 (s, 3H), 2.19-2.02 (m, 2H), 1.36-1.06 (m, |
| | 14H), 1H not observed (NH); LC/MS (ESI): 479.2 [M + H]⁺ |
| 151 | LC/MS (ESI): 417.2 [M + H]⁺ |
| 152 | LC/MS (ESI): 417.2 [M + H]⁺ |
| 153 | LC/MS (ESI): 445.2 [M + H]⁺ |
| 154 | LC/MS (ESI): 433.5 [M + H]⁺ |
| 155 | ¹H NMR (DMSO-d₆, 400 MHz) δ = 9.12 (d, J = 1.6 Hz, 1H), 8.49 (d, J = 9.8 |
| | Hz, 1H), 8.44 (s, 1H), 8.36 (d, J = 1.8 Hz, 1H), 8.14 (s, 1H), 7.51 (d, J = 10.0 |
| | Hz, 1H), 5.13 (s, 2H), 4.27 (br d, J = 11.1 Hz, 1H), 4.15 (br d, J = 10.8 Hz, |
| | 1H), 3.89 (s, 3H), 3.14-3.03 (m, 2H), 2.95 (br t, J = 11.5 Hz, 1H), 2.84-2.74 |
| | (m, 2H), 0.84 (br d, J = 7.1 Hz, 1H), 0.48 (br s, 2H), 0.33 (br s, 2H), 1H not |
| | observed (NH); LC/MS (ESI): 417.2 [M + H]⁺ |
| 156 | ¹H NMR (400 MHZ, DMSO-d₆) δ = 8.93 (d, J = 2.0 Hz, 1H), 8.48 (d, J = 9.9 |
| | Hz, 1H), 8.30 (br s, 1H), 8.17 (s, 1H), 8.14 (d, J = 2.0 Hz, 1H), 7.33 (d, J = |
| | 10.0 Hz, 1H), 5.17 (s, 2H), 5.06 (br t, J = 11.8 Hz, 1H), 3.82 (s, 3H), 2.91 (s, |
| | 3H), 2.37-2.34 (m, 5H), 1.69-1.52 (m, 3H), 1.35 (s, 6H), 1.22 (s, 6H); |
| | LC/MS (ESI): 475.3 [M + H]⁺ |
| 157 | ¹H NMR (DMSO-d₆, 400 MHZ) δ 9.12 (d, 1H, J = 2.0 Hz), 8.48 (d, 1H, J = |
| | 10.0 Hz), 8.44 (s, 1H), 8.37 (d, 1H, J = 2.1 Hz), 8.18 (s, 1H), 8.14 (s, 1H), 7.48 |
| | (d, 1H, J = 9.9 Hz), 5.14 (s, 2H), 4.27-4.34 (m, 1H), 4.11-4.19 (m, 1H), 3.90 |
| | (s, 3H), 3.02-3.15 (m, 1H), 2.82-2.92 (m, 1H), 2.79-2.86 (m, 1H), 2.62-2.72 |
| | (m, 2H), 1.66-1.72 (m, 1H), 0.97 (d, 6H, J = 6.8 Hz); LC/MS (ESI): 419.2 |
| | [M + H]⁺ |
| 158 | LC/MS (ESI): 431.3 [M + H]⁺ |
| 159 | LC/MS (ESI): 431.3 [M + H]⁺ |
| 160 | LC/MS (ESI): 497.3 [M + H]⁺ |
| 161 | LC/MS (ESI): 464.3 [M + H]⁺ |
| 162 | ¹H NMR (DMSO-d₆, 400 MHz) δ = 9.41 (s, 1H), 8.95 (d, J = 2.0 Hz, 1H), 8.22- |
| | 8.14 (m, 2H), 5.14 (m, 3H), 3.83 (s, 3H), 3.09 (s, 3H), 2.35 (s, 3H), 1.99- |
| | 1.82 (m, 4H), 1.51 (s, 6H), 1.43 (s, 6H), 1H not observed (NH); LC/MS (ESI): |
| | 476.3 [M + H]⁺ |
| 163 | LC/MS (ESI): 501.3 [M + H]⁺ |
| 164 | 1H NMR (DMSO-d₆ 400 MHZ) δ = 8.94 (s, 1H), 8.85-8.66 (m, 2H), 8.53 (d, |
| | J = 9.9 Hz, 1H), 8.19-8.13 (m, 2H), 7.44 (br d, J = 10.0 Hz, 1H), 5.15 (s, 2H), |
| | 5.05 (td, J = 5.7, 11.5 Hz, 1H), 3.82 (s, 3H), 2.94 (s, 3H), 2.36 (s, 3H), 2.18 (br |
| | d, J = 8.3 Hz, 2H), 2.03-1.93 (m, 2H), 1.90 (br d, J = 8.3 Hz, 2H), 1.83 (br |
| | dd, J = 5.4, 13.2 Hz, 2H), 1.44 (s, 6H); LC/MS (ESI): 473.2 [M + H]⁺ |
| 165 | LC/MS (ESI): 417.3 [M + H]⁺ |
| 166 | ¹H NMR (DMSO-d₆, 400 MHZ) δ = 8.99-8.88 (m, 1H), 8.47 (d, J = 9.9 Hz, |
| | 1H), 8.40-8.26 (m, 1H), 8.20 (d, J = 2.0 Hz, 1H), 7.33 (d, J = 10.0 Hz, 1H), |
| | 5.21 (s, 2H), 5.02 (td, J = 5.7, 11.3 Hz, 1H), 2.90 (s, 3H), 2.68 (s, 3H), 2.43 (s, |
| | 3H), 1.98 (br d, J = 7.6 Hz, 2H), 1.85-1.74 (m, 2H), 1.73-1.59 (m, 4H), 1.31 |
| | (s, 6H); LC/MS (ESI): 490.3 [M + H]⁺ |
| 167 | LC/MS (ESI): 492.3 [M + H]⁺ |
| 168 | LC/MS (ESI): 501.3 [M + H]⁺ |
| 169 | ¹H NMR (DMSO-d₆, 400 MHz) δ = 9.26 (d, J = 2.1 Hz, 1H), 9.06 (s, 1H), 8.56 |
| | (s, 1H), 8.56 (d, J = 3.8 Hz, 2H), 8.48 (d, J = 9.9 Hz, 1H), 8.38 (br s, 1H), 8.07- |
| | 7.70 (m, 1H), 7.31 (d, J = 10.0 Hz, 1H), 5.17 (s, 2H), 5.10-4.95 (m, 1H), |
| | 2.90 (s, 3H), 1.95 (br d, J = 7.5 Hz, 2H), 1.84-1.73 (m, 2H), 1.73-1.57 (m, |
| | 4H), 1.31 (s, 6H); LC/MS (ESI): 495.3 [M + H]⁺ |
| 170 | LC/MS (ESI): 431.2 [M + H]⁺ |
| 171 | ¹H NMR (400 MHZ, DMSO-d₆) δ = 8.97-8.92 (m, 1H), 8.70 (d, J = 9.6 Hz, |
| | 1H), 8.40 (s, 1H), 8.19 (d, J = 1.9 Hz, 1H), 8.11 (s, 1H), 7.33 (d, J = 9.6 Hz, |
| | 1H), 5.54-5.43 (m, 1H), 5.18 (s, 2H), 3.84 (s, 3H), 2.37 (s, 5H), 2.06 (br d, J = |
| | 7.8 Hz, 2H), 1.82 (br d, J = 8.1 Hz, 2H), 1.70 (br t, J = 11.8 Hz, 2H), 1.39 (s, |
| | 6H); LC/MS (ESI): 460.3 [M + H]⁺ |
| 172 | LC/MS (ESI): 530.2 [M + H]⁺ |
| 173 | LC/MS (ESI): 543.3 [M + H]⁺ |
| 174 | ¹HNMR (DMSO-d₆, 400 MHz) δ = 8.95 (d, J = 1.8 Hz, 1H), 8.68 (d, J = 9.6 |
| | Hz, 1H), 8.24 (d, J = 1.9 Hz, 1H), 7.32 (d, J = 9.6 Hz, 1H), 5.53-5.41 (m, |
| | 1H), 5.23 (s, 2H), 2.67 (s, 3H), 2.43 (s, 3H), 2.16 (dd, J = 5.9, 11.9 Hz, 2H), |
| | 1.80 (br d, J = 7.0 Hz, 2H), 1.53 (br d, J = 7.4 Hz, 2H), 1.41 (br t, J = 11.3 Hz, |
| | 2H), 1.20 (s, 6H), 1H not observed (NH); LC/MS (ESI): 477.2 [M + H]⁺ |
| 175 | ¹H NMR (DMSO-d₆, 400 MHz) δ = 9.34 (s, 1H), 8.94 (d, J = 1.6 Hz, 1H), 8.18 |
| | (s, 2H), 5.13 (s, 2H), 5.01-5.10 (m, 1H), 3.82 (s, 3H), 3.02 (s, 3H), 2.36 (s, |
| | 3H), 1.78 (br d, J = 7.4 Hz, 2H), 1.51-1.65 (m, 6H), 1.20 (s, 6H), 1H not |
| | observed (NH); LC/MS (ESI): 474.3 [M + H]⁺ |
| 176 | LC/MS (ESI): 516.1 [M + H]⁺ |
| 177 | LC/MS (ESI): 529.2 [M + H]⁺ |
| 178 | LC/MS (ESI): 474.2 [M + H]⁺ |
| 179 | LC/MS (ESI): 463.2 [M + H]⁺ |
| 180 | ¹H NMR (DMSO-d₆, 400 MHz) δ = 9.14 (d, J = 2.0 Hz, 1H), 8.49 (d, J = 10.0 |
| | Hz, 1H), 8.33 (d, J = 1.9 Hz, 1H), 8.32-8.19 (m, 1H), 7.34 (d, J = 10.0 Hz, |
| | 1H), 5.22 (s, 2H), 5.09 (br s, 1H), 4.18 (s, 3H), 2.91 (s, 3H), 2.49 (br s, 3H), |
| | 1.75-1.59 (m, 4H), 1.39 (s, 6H), 1.27 (s, 6H); LC/MS (ESI): 476.2 [M + H]⁺ |
| 181 | LC/MS (ESI): 478.2 [M + H]⁺ |
| 182 | LC/MS (ESI): 515.2 [M + H]⁺ |
| 183 | ¹HNMR (DMSO-d₆, 400MHz) δ = 8.91 (d, J = 1.8 Hz, 1H), 8.75-8.63 (m, |
| | 1H), 8.30 (s, 1H), 8.19 (s, 1H), 7.71 (br d, J = 10.3 Hz, 1H), 5.17 (s, 2H), 4.85 |
| | (br s, 1H), 4.01 (br s, 3H), 3.00 (s, 3H), 2.23 (br d, J = 8.4 Hz, 2H), 2.06-1.84 |
| | (m, 6H), 1.46 (s, 6H), 1H not observed (NH); LC/MS (ESI): 527.2 [M + H]⁺ |
| 184 | LC/MS (ESI): 498.2 [M + H]⁺ |
| 185 | LC/MS (ESI): 474.2 [M + H]⁺ |
| 186 | LC/MS (ESI): 460.2 [M + H]⁺ |
| 187 | ¹H NMR (DMSO-d₆, 400 MHZ) δ = 8.98-9.05 (m, 1H), 8.45-8.51 (m, 1H), |
| | 8.40-8.44 (m, 1H), 8.31-8.36 (m, 1H), 8.20-8.25 (m, 1H), 7.21-7.41 (m, 1H), |
| | 5.16 (s, 2H), 5.03-5.09 (m, 1H), 3.80 (s, 3H), 2.91 (s, 3H), 1.55-1.70 (m, 4H), |
| | 1.33-1.40 (m, 6H), 1.23 (s, 6H); LC/MS (ESI): 479.6 [M + H] + |
| 188 | LC/MS (ESI): 460.2 [M + H]⁺ |
| 189 | LC/MS (ESI): 462.3 [M + H]⁺ |
| 190 | ¹H NMR (DMSO-d₆, 400 MHz) δ = 8.99-9.09 (m, 1H), 8.68 (d, J = 9.6 Hz, |
| | 1H), 8.37-8.47 (m, 1H), 8.22-8.31 (m, 1H), 7.24-7.36 (m, 1H), 5.57-5.68 (m, |
| | 1H), 5.19 (s, 2H), 3.80 (s, 3H), 2.05-2.07 (m, 2H), 1.24-1.29 (m, 2H), 1.23 (s, |
| | 6H), 1.08-1.13 (m, 6H), 1H not observed (NH); LC/MS (ESI): 466.2 [M + H] + |
| 191 | ¹H NMR (DMSO-d₆, 400 MHz) δ = 9.33 (s, 1H), 8.71 (br d, J = 9.5 Hz, 1H), |
| | 8.58 (br s, 1H), 8.53 (s, 1H), 7.35 (br d, J = 9.5 Hz, 1H), 5.64-5.67 (m, 1H), |
| | 5.21 (s, 2H), 4.25 (s, 3H), 2.12-2.22 (m, 2H), 1.53-1.57 (m, 2H), 1.41- |
| | 1.5270m, 6H), 1.23-1.32(m, 6H), 1H not observed (NH); LC/MS (ESI): 449.2 |
| | [M + H]⁺ |
| 193 | LC/MS (ESI): 492.3 [M + H]⁺ |
| 194 | LC/MS (ESI): 477.2 [M + H]⁺ |
| 195 | LC/MS (ESI): 493.2 [M + H]⁺ |
| 196 | LC/MS (ESI): 490.2 [M + H]⁺ |
| 197 | LC/MS (ESI): 477.2 [M + H]⁺ |
| 198 | LC/MS (ESI): 511.2 [M + H]⁺ |
| 199 | ¹H NMR (DMSO-d₆, 400 MHz) δ = 9.34 (d, J = 1.3 Hz, 1H), 8.70 (d, J = 9.5 |
| | Hz, 1H), 8.60 (d, J = 1.3 Hz, 1H), 8.54 (s, 1H), 7.32 (br d, J = 9.6 Hz, 1H), |
| | 5.49 (br s, 1H), 5.22 (s, 2H), 4.25 (s, 3H), 2.10 (br d, J = 7.0 Hz, 2H), 1.89- |
| | 1.99 (m, 2H), 1.74-1.85 (m, 2H), 1.50 (br d, J = 6.6 Hz, 2H), 1.13-1.20 (s, 6H), |
| | 1H not observed (NH); LC/MS (ESI): 447.2 [M + H]⁺ |
| 200 | ¹HNMR (DMSO-d₆, 400 MHz) δ = 9.35 (s, 1H), 8.94 (d, J = 2.1 Hz, 1H), 8.24 |
| | (d, J = 2.1 Hz, 1H), 5.18 (s, 2H), 5.01-5.12 (m, 1H), 3.03 (s, 3H), 2.68 (s, 3H), |
| | 2.43 (s, 3H), 1.83 (br d, J = 7.4 Hz, 2H), 1.56-1.70 (m, 6H), 1.24 (s, 6H), 1H |
| | not observed (NH); LC/MS (ESI): 491.2 [M + H]⁺ |
| 201 | LC/MS (ESI): 514.2 [M + H]⁺ |
| 202 | LC/MS (ESI): 514.2 [M + H]⁺ |
| 203 | LC/MS (ESI): 492.2 [M + H]⁺ |
| 205 | ¹H NMR (DMSO-d₆, 400 MHz) δ = 8.95 (d, J = 2.1 Hz, 1H), 8.69 (d, J = 9.6 |
| | Hz, 1H), 8.19-8.15 (m, 2H), 7.31 (d, J = 9.6 Hz, 1H), 5.61-5.52 (m, 1H), |
| | 5.19 (s, 2H), 3.82 (s, 3H), 2.36 (s, 3H), 2.09 (br dd, J = 3.3, 11.9 Hz, 1H), 2.02 |
| | (br s, 1H), 1.80 (t, J = 11.4 Hz, 1H), 1.60 (dd, J = 3.9, 12.0 Hz, 1H), 1.32 (t, J = |
| | 11.6 Hz, 1H), 1.19 (s, 3H), 1.07 (s, 3H), 0.55-0.48 (m, 2H), 0.46-0.39 (m, |
| | 1H), 0.36-0.27 (m, 1H); LC/MS (ESI): 460.2 [M + H]⁺ |
| 206 | ¹H NMR (DMSO-d₆, 400 MHZ) δ = 9.18 (d, J = 2.0 Hz, 1H), 8.87 (br d, J = |
| | 10.6 Hz, 1H), 8.79 (br s, 1H), 8.75 (d, J = 9.6 Hz, 1H), 8.39 (d, J = 2.0 Hz, |
| | 1H), 7.41 (d, J = 9.5 Hz, 1H), 5.55 (tt, J = 10.9, 5.6 Hz, 1H), 5.24 (s, 2H), 4.19 |
| | (s, 3H), 2.46 (br d, J = 6.1 Hz, 2H), 2.18 (br d, J = 8.0 Hz, 2H), 1.90 (br d, J = |
| | 8.3 Hz, 2H), 1.80 (br t, J = 12.1 Hz, 2H), 1.46 (s, 6H), 3H not observed |
| | (triazole methyl overlapped with DMSO-d6 residue); LC/MS (ESI): 461.2 |
| | [M + H]⁺ |
| 207 | ¹HNMR (DMSO-d₆, 400 MHz) δ = 8.91 (d, J = 2.0 Hz, 1H), 8.39 (d, J = 9.6 |
| | Hz, 2H), 8.09-8.16 (m, 2H), 6.99 (d, J = 9.6 Hz, 1H), 5.12 (s, 2H), 4.33-4.45 |
| | (m, 1H), 3.82 (s, 3H), 2.35 (s, 3H), 2.07 (br dd, J = 12.9, 2.9 Hz, 2H), 1.34- |
| | 1.44 (m, 8H), 1.32 (s, 6H), 1H not observed (NH); LC/MS (ESI): 461.3 |
| | [M + H]⁺ |
| 208 | LC/MS (ESI): 496.2 [M + H]⁺ |
| 209 | LC/MS (ESI): 494.2 [M + H]⁺ |
| 210 | LC/MS (ESI): 498.2 [M + H]⁺ |
| 211 | ¹H NMR (DMSO-d₆, 400 MHz) δ = 8.96 (d, J = 2.1 Hz, 1H), 8.75 (d, J = 9.6 |
| | Hz, 1H), 8.19 (s, 2H), 7.44 (d, J = 9.6 Hz, 1H), 5.89-5.76 (m, 1H), 5.20 (s, |
| | 2H), 4.75 (d, J = 52.0 Hz, 1H), 3.83 (s, 3H), 2.37 (s, 3H), 2.00-1.88 (m, 1H), |
| | 1.77-1.66 (m, 1H), 1.30 (br s, 6H), 1.19 (br s, 6H), 1H not observed (NH); |
| | LC/MS (ESI): 480.2 [M + H]⁺ |
| 212 | LC/MS (ESI): 480.2 [M + H]⁺ |
| 213 | LC/MS (ESI): 496.2 [M + H]⁺ |
| 214 | ¹H NMR (DMSO-d₆, 400 MHz) δ = 8.94-9.03 (m, 1H), 8.75 (d, J = 9.6 Hz, |
| | 1H), 8.19 (s, 2H), 7.43 (d, J = 9.6 Hz, 1H), 5.60 (s, 1H), 5.20 (s, 2H), 4.63- |
| | 4.87 (m, 1H), 3.83 (s, 3H), 2.37 (s, 3H), 1.98-2.12 (m, 1H), 1.47-1.84 (m, 5H), |
| | 1.17-1.35 (s, 6H), 1H not observed (NH); LC/MS (ESI): 478.5 [M + H]⁺ |
| 215 | LC/MS (ESI): 478.5 [M + H]⁺ |
| 216 | LC/MS (ESI): 492.2 [M + H]⁺ |
| 217 | ¹H NMR (DMSO-d₆, 400 MHz) δ: 9.35 (s, 1H), 9.04 (s, 1H), 8.43 (s, 1H), 8.34 |
| | (br s, 1H), 8.23-8.31 (m, 1H), 5.12-5.17 (m, 2H), 5.04-5.11 (m, 1H), 3.81 (s, |
| | 3H), 3.04 (s, 3H), 1.87 (br d, J = 6.9 Hz, 2H), 1.72-1.80 (m, 2H), 1.56-1.67 (m, |
| | 4H), 1.27-1.32 (m, 6H); LC/MS (ESI): 478.2 [M + H]⁺ |
| 218 | LC/MS (ESI): 499.2 [M + H]⁺ |
| 219 | LC/MS (ESI): 461.2 [M + H]⁺ |
| 220 | LC/MS (ESI): 443.2 [M + H]⁺ |
| 221 | LC/MS (ESI): 475.3 [M + H]⁺ |
| 222 | LC/MS (ESI): 448.2 [M + H]⁺ |
| 223 | ¹H NMR (DMSO-d₆, 400 MHZ) δ = 9.41 (s, 1H), 9.05 (d, J = 2.0 Hz, 1H), 8.71 |
| | (br d, J = 12.3 Hz, 1H), 8.44 (d, J = 2.1 Hz, 1H), 8.28 (d, J = 2.1 Hz, 1H), |
| | 7.70-7.82 (m, 1H), 5.16-5.19 (m, 1H), 5.15 (s, 2H), 3.82 (s, 3H), 3.10 (s, 3H), |
| | 1.90-1.98 (m, 2H), 1.81-1.87 (m, 2H), 1.51 (s, 6H), 1.43 (s, 6H); LC/MS |
| | (ESI): 480.2 [M + H]⁺ |
| 224 | ¹H NMR (DMSO-d₆, 400 MHz) δ = 9.70 (s, 1H), 8.94 (d, J = 1.0 Hz, 1H), |
| | 8.17-8.17 (m, 1H), 8.07 (s, 1H), 5.48-5.62 (m, 1H), 5.16 (s, 2H), 3.83 (s, 3H), |
| | 2.36 (s, 3H), 2.27-2.33 (m, 2H), 1.49 (br s, 8H), 1.39-1.47 (m, 6H), 1H not |
| | observed (NH); LC/MS (ESI): 463.3 [M + H]⁺ |
| 225 | LC/MS (ESI): 505.2 [M + H]⁺ |
| 226 | LC/MS (ESI): 434.2 [M + H]⁺ |
| 227 | ¹H NMR (DMSO-d₆, 400 MHZ) δ = 9.50 (d, J = 1.9 Hz, 1H), 9.36-9.25 (m, |
| | 1H), 9.16-9.07 (m, 1H), 8.77-8.73 (m, 2H), 7.41 (d, J = 9.6 Hz, 1H), 5.59- |
| | 5.49 (m, 1H), 5.29 (s, 2H), 2.72 (s, 3H), 2.47-2.39 (m, 2H), 2.25-2.06 (m, |
| | 2H), 1.95-1.89 (m, 2H), 1.89-1.82 (m, 2H), 1.48 (s, 6H); LC/MS (ESI): |
| | 464.2 [M + H]⁺ |
| 228 | ¹H NMR (DMSO-d₆, 400 MHZ) δ = 9.10 (d, J = 2.0 Hz, 2H), 8.68 (d, J = 9.6 |
| | Hz, 1H), 8.56 (br d, J = 11.6 Hz, 1H), 8.31 (d, J = 2.0 Hz, 1H), 7.33 (d, J = 9.6 |
| | Hz, 1H), 5.51-5.59 (m, 1H), 5.17 (s, 2H), 4.11 (s, 3H), 2.42 (br s, 3H), 2.29 (br |
| | dd, J = 13.7, 3.6 Hz, 1H), 2.00-2.12 (m, 1H), 1.94 (br d, J = 3.5 Hz, 1H), 1.68- |
| | 1.83 (m, 1H), 1.40 (s, 3H), 1.34 (s, 3H), 0.84-1.01 (m, 3H), 0.77 (br d, J = 3.3 |
| | Hz, 1H); LC/MS (ESI): 461.2 [M + H]⁺ |
| 230 | LC/MS (ESI): 462.3 [M + H]⁺ |
| 231 | LC/MS (ESI): 477.2 [M + H]⁺ |
| 232 | ¹H NMR (400 MHZ, DMSO-d₆) δ = 9.03 (s, 1H), 8.70 (s, 1H), 8.48 (d, 1H, J = |
| | 9.9 Hz), 8.3-8.3 (m, 1H), 7.96 (s, 1H), 7.33 (d, 1H, J = 10.0 Hz), 5.27 (s, 2H), |
| | 5.0-5.1 (m, 1H), 4.28 (d, 2H, J = 7.4 Hz), 2.91 (s, 3H), 1.59 (br d, 4H, J = 5.5 |
| | Hz), 1.47 (t, 3H, J = 7.3 Hz), 1.34 (s, 6H), 1.21 (br s, 6H); 1H not observed |
| | (NH); LC/MS (ESI): 458.3 [M + H]⁺ |
| 233 | LC/MS (ESI): 464.1 [M + H]⁺ |
| 234 | LC/MS (ESI): 448.5 [M + H]⁺ |
| 235 | LC/MS (ESI): 476.3 [M + H]⁺ |
| 236 | LC/MS (ESI): 418.2 [M + H]⁺ |
| 237 | LC/MS (ESI): 463.2 [M + H]⁺ |
| 238 | LC/MS (ESI): 474.2 [M + H]⁺ |
| 239 | ¹H NMR (METHANOL-d₄, 400 MHz) d: 8.94 (d, J = 2.0 Hz, 1H), 8.88 (d, J = |
| | 9.6 Hz, 1H), 8.29 (d, J = 1.9 Hz, 1H), 7.98 (s, 1H), 7.28 (d, J = 9.6 Hz, 1H), |
| | 5.72-5.59 (m, 1H), 5.25 (s, 2H), 3.91 (s, 3H), 2.66 (dd, J = 5.9, 13.9 Hz, 2H), |
| | 2.44 (s, 3H), 2.39-2.31 (m, 2H), 1.99 (br d, J = 8.4 Hz, 2H), 1.92-1.86 (m, |
| | 4H), 1.82 (br d, J = 13.3 Hz, 2H), 1.08 (t, J = 7.6 Hz, 6H), 1H not observed |
| | (NH); LC/MS (ESI): 488.3 [M + H]⁺ |
| 240 | ¹H NMR (DMSO-d₆, 400 MHZ) δ 8.9-9.0 (m, 1H), 8.7-8.8 (m, 1H), 8.2-8.2 (m, |
| | 2H), 7.4-7.4 (m, 1H), 5.5-5.6 (m, 1H), 5.1-5.2 (m, 2H), 3.82 (s, 3H), 2.4-2.5 |
| | (m, 2H), 2.3-2.4 (m, 3H), 2.1-2.2 (m, 2H), 1.7-1.9 (m, 6H), 1.4-1.5 (m, 3H), |
| | 0.9-1.0 (m, 3H); LC/MS (ESI): 474.2 [M + H]⁺ |
| 241 | LC/MS (ESI): 486.2 [M + H]⁺ |
| 242 | LC/MS (ESI): 474.2 [M + H]⁺ |
| 243 | LC/MS (ESI): 542.4 [M + H]⁺ |
| 244 | LC/MS (ESI): 461.2 [M + H]⁺ |
| 245 | LC/MS (ESI): 486.2 [M + H]⁺ |
| 246 | LC/MS (ESI): 478.2 [M + H]⁺ |
| 247 | LC/MS (ESI): 490.2 [M + H]⁺ |
| 248 | LC/MS (ESI): 463.2 [M + H]⁺ |
| 249 | ¹H NMR (DMSO-d₆, 400 MHz) δ = 8.95 (d, J = 2.0 Hz, 1H), 8.71 (d, J = 9.6 |
| | Hz, 1H), 8.22 (s, 1H), 8.19-8.15 (m, 2H), 7.35 (d, J = 9.6 Hz, 1H), 5.57-5.46 |
| | (m, 1H), 5.20 (s, 2H), 3.82 (s, 3H), 2.57-2.52 (m, 1H), 2.36 (s, 3H), 2.21- |
| | 2.07 (m, 4H), 1.94-1.85 (m, 1H), 1.83-1.65 (m, 2H), 1.55-1.40 (m, 2H), |
| | 1.21 (s, 3H), 1.16 (s, 3H); LC/MS (ESI): 474.2 [M + H]⁺ |
| 250 | LC/MS (ESI): 463.0 [M + H]⁺ |
| 251 | ¹H NMR (C2DF302, 400 MHZ) δ = 9.90 (s, 1H), 9.64 (s, 1H), 8.89 (br d, J = |
| | 9.6 Hz, 1H), 8.70 (s, 1H), 8.24 (br d, J = 9.6 Hz, 1H), 7.72-7.49 (m, 1H), 7.39- |
| | 7.14 (m, 1H), 6.07 (br s, 2H), 5.79 (br d, J = 5.1 Hz, 1H), 4.59 (s, 3H), 2.79 |
| | (br dd, J = 5.3, 13.8 Hz, 2H), 2.52-2.37 (m, 6H), 1.82 (s, 6H); LC/MS (ESI): |
| | 447.2 [M + H]⁺ |
| 252 | ¹H NMR (DMSO-d₆, 400 MHZ) δ = 9.14 (d, J = 1.9 Hz, 1H), 8.47 (d, J = 10.0 |
| | Hz, 1H), 8.33 (d, J = 1.5 Hz, 1H), 8.18 (br s, 1H), 7.48 (d, J = 10.0 Hz, 1H), |
| | 5.20 (s, 2H), 4.37 (br d, J = 12.1 Hz, 1H), 4.18 (s, 3H), 4.12 (br d, J = 12.1 Hz, |
| | 1H), 3.09-3.05 (m, 1H), 2.87-2.80 (m, 1H), 2.78-2.70 (m, 1H), 2.60 (br t, J = |
| | 11.6 Hz, 1H), 2.49 (br s, 3H), 2.36 (br d, J = 10.4 Hz, 1H), 0.96 (s, 9H); |
| | LC/MS (ESI): 448.2 [M + H]⁺ |
| 253 | LC/MS (ESI): 461.2 [M + H]⁺ |
| 254 | LC/MS (ESI): 433.3 [M + H]⁺ |
| 255 | LC/MS (ESI): 422.95 [M + H]⁺ |
| 272 | LC/MS (ESI): 434.3 [M + H]⁺ |
| 273 | LC/MS (ESI): 476.2 [M + H]⁺ |
| 274 | ¹H NMR (DMSO-d₆, 400 MHz) δ = 8.51-8.33 (m, 2H), 8.04 (s, 1H), 7.64- |
| | 7.60 (m, 1H), 7.55 (d, J = 10.3 Hz, 1H), 7.31 (d, J = 10.0 Hz, 1H), 5.13 (s, |
| | 2H), 5.11-5.03 (m, 1H), 3.81 (s, 3H), 2.90 (s, 3H), 2.36 (s, 3H), 2.16 (br d, J = |
| | 8.1 Hz, 2H), 2.03-1.87 (m, 4H), 1.80 (br dd, J = 5.4, 13.6 Hz, 2H), 1.45 (s, |
| | 6H); LC/MS (ESI): 490.2 [M + H]⁺ |
| 275 | ¹HNMR (DMSO-d₆, 400 MHz) δ = 8.48-8.39 (m, 1H), 8.36 (d, J = 9.9 Hz, |
| | 1H), 7.99 (s, 1H), 7.55 (s, 1H), 7.49 (d, J = 10.4 Hz, 1H), 7.23 (d, J = 10.1 Hz, |
| | 1H), 5.14 (br t, J = 11.9 Hz, 1H), 5.02 (s, 2H), 3.79 (s, 3H), 2.89 (s, 3H), 2.33 |
| | (s, 3H), 1.95-1.82 (m, 2H), 1.75 (br d, J = 11.0 Hz, 2H), 1.54 (s, 6H), 1.43 (s, |
| | 6H); LC/MS (ESI): 492.3 [M + H]⁺ |
| 276 | ¹H NMR (METHANOL-d₄, 400 MHZ) δ = 8.84 (d, J = 9.6 Hz, 1H), 8.52 (br s, |
| | 1H), 7.90 (s, 1H), 7.75-7.72 (m, 1H), 7.53 (dd, J = 0.9, 10.2 Hz, 1H), 7.26 (d, |
| | J = 9.6 Hz, 1H), 5.77 (tt, J = 4.1, 10.5 Hz, 1H), 5.22 (s, 2H), 3.89 (s, 3H), 2.47 |
| | (dd, J = 4.1, 13.8 Hz, 2H), 2.43 (s, 3H), 1.84 (dd, J = 10.9, 13.3 Hz, 2H), 1.64 |
| | (s, 6H), 1.56 (s, 6H); LC/MS (ESI): 479.2 [M + H]⁺ |
| 277 | LC/MS (ESI): 493.3 [M + H]⁺ |
| 278 | ¹H NMR (DMSO-d₆, 400 MHZ) δ = 8.68 (d, J = 9.6 Hz, 1H), 8.36 (s, 1H), 8.09 |
| | (s, 1H), 7.65 (s, 1H), 7.60 (d, J = 10.4 Hz, 1H), 7.33 (d, J = 9.8 Hz, 1H), 5.55- |
| | 5.40 (m, 1H), 5.20 (s, 2H), 3.79 (s, 3H), 2.39-2.31 (m, 5H), 2.04 (br d, J = 7.9 |
| | Hz, 2H), 1.81 (br d, J = 8.0 Hz, 2H), 1.72 (br t, J = 11.9 Hz, 2H), 1.38 (s, 6H); |
| | LC/MS (ESI): 477.2 [M + H]⁺ |
| 279 | LC/MS (ESI): 497.2 [M + H]⁺ |
| 280 | ¹HNMR (CHLOROFORM-d, 400 MHz) δ = 8.92 (d, J = 9.6 Hz, 1H), 8.31 (br |
| | s, 1H), 7.75 (d, J = 1.0 Hz, 1H), 7.56 (s, 1H), 7.37 (dd, J = 1.2, 9.8 Hz, 1H), |
| | 7.19 (d, J = 9.6 Hz, 1H), 6.02-5.86 (m, 1H), 5.28 (s, 2H), 4.87-4.67 (m, J = |
| | 52.8 Hz, 1H), 3.93 (s, 3H), 2.46 (s, 3H), 2.05 (br d, J = 7.8 Hz, 2H), 1.55- |
| | 1.50 (m, 6H), 1.43 (s, 6H); LC/MS (ESI): 497.2 [M + H]⁺ |
| 281 | LC/MS (ESI): 495.2 [M + H]⁺ |
| 282 | LC/MS (ESI): 480.2 [M + H]⁺ |
| 283 | ¹H NMR (400 MHZ, DMSO-d₆) δ: 9.42 (br d, J = 7.4 Hz, 1H), 9.31-9.17 (m, |
| | 1H), 8.72 (d, J = 9.6 Hz, 1H), 7.89 (s, 1H), 7.83 (dd, J = 0.8, 9.9 Hz, 1H), 7.39 |
| | (d, J = 9.6 Hz, 1H), 5.52 (td, J = 5.3, 10.8 Hz, 1H), 5.27 (s, 2H), 4.16 (s, 3H), |
| | 2.48 (s, 3H), 2.41 (br dd, J = 5.8, 13.3 Hz, 2H), 2.13 (br d, J = 8.0 Hz, 2H), |
| | 1.95-1.81 (m, 4H), 1.48 (s, 6H); LC/MS (ESI): 478.2 [M + H]⁺ |
| 284 | ¹HNMR (400 MHZ, DMSO-d₆) δ = 8.68 (d, J = 9.6 Hz, 1H), 8.14 (s, 1H), 7.67 |
| | (s, 1H), 7.62 (d, J = 10.4 Hz, 1H), 7.31 (d, J = 9.5 Hz, 1H), 5.62-5.52 (m, |
| | 1H), 5.23 (s, 2H), 3.80 (s, 3H), 2.36 (s, 3H), 2.11 (br dd, J = 3.3, 12.1 Hz, 1H), |
| | 1.83 (br t, J = 11.4 Hz, 1H), 1.67-1.58 (m, 1H), 1.36 (br t, J = 11.0 Hz, 1H), |
| | 1.21 (s, 3H), 1.10 (s, 3H), 0.54 (br d, J = 6.4 Hz, 2H), 0.47 (br d, J = 4.8 Hz, |
| | 1H), 0.36 (br s, 1H), 1H not observed (NH); LC/MS (ESI): 477.2 [M + H]⁺ |
| 285 | LC/MS (ESI): 515.2 [M + H]⁺ |
| 286 | LC/MS (ESI): 494.2 [M + H]⁺ |
| 287 | LC/MS (ESI): 496.2 [M + H]⁺ |
| 288 | LC/MS (ESI): 497.2 [M + H]⁺ |
| 289 | ¹H NMR (DMSO-d₆, 400 MHZ) δ = 8.45 (d, J = 9.9 Hz, 1H), 8.35 (s, 1H), 7.67 |
| | (d, J = 9.8 Hz, 1H), 7.64 (s, 1H), 7.33 (d, J = 10.0 Hz, 1H), 5.25 (s, 2H), 5.06- |
| | 4.95 (m, 1H), 2.91 (s, 3H), 2.66 (s, 3H), 2.45-2.42 (m, 3H), 1.96 (br d, J = 7.3 |
| | Hz, 2H), 1.77 (br d, J = 12.1 Hz, 2H), 1.69 (br d, J = 7.6 Hz, 2H), 1.63 (br dd, |
| | J = 5.7, 12.7 Hz, 2H), 1.31 (s, 6H); LC/MS (ESI): 507.2 [M + H]⁺ |
| 290 | LC/MS (ESI): 509.2 [M + H]⁺ |
| 291 | ¹HNMR (METHANOL-d₄, 400 MHZ) δ = 9.57-9.47 (m, 1H), 8.60-8.48 (m, |
| | 1H), 7.90 (s, 1H), 7.76 (s, 1H), 7.53 (d, J = 10.1 Hz, 1H), 5.29-5.21 (m, 1H), |
| | 5.19 (s, 2H), 3.92-3.85 (m, 3H), 3.15 (s, 3H), 2.42 (s, 3H), 2.27 (br d, J = 8.3 |
| | Hz, 2H), 2.05-1.92 (m, 6H), 1.52 (s, 6H); LC/MS (ESI): 491.3 [M + H]⁺ |
| 292 | LC/MS (ESI): 480.2 [M + H]⁺ |
| 293 | LC/MS (ESI): 493.2 [M + H]⁺ |
| 294 | ¹H NMR (DMSO-d₆, 400 MHz) δ = 8.47 (d, J = 10.0 Hz, 1H), 8.27 (s, 1H), |
| | 7.88 (s, 1H), 7.81 (d, J = 9.9 Hz, 1H), 7.34 (d, J = 10.0 Hz, 1H), 5.26 (s, 2H), |
| | 5.08-4.95 (m, 1H), 4.16 (s, 3H), 2.91 (s, 3H), 2.49 (br s, 3H), 1.99 (br d, J = |
| | 7.8 Hz, 2H), 1.80-1.62 (m, 6H), 1.32 (s, 6H); LC/MS (ESI): 491.2 [M + H] + |
| 295 | ¹H NMR (DMSO-d₆, 400 MHz) δ = 8.46 (d, J = 9.9 Hz, 1H), 8.13 (s, 1H), 7.64 |
| | (s, 1H), 7.61-7.56 (m, 1H), 7.47 (d, J = 10.0 Hz, 1H), 5.18 (s, 2H), 4.36 (br d, |
| | J = 12.1 Hz, 1H), 4.12 (br d, J = 11.9 Hz, 1H), 3.80 (s, 3H), 3.08 (br d, J = |
| | 11.4 Hz, 1H), 2.89-2.71 (m, 2H), 2.65-2.57 (m, 1H), 2.39 (dd, J = 2.4, 10.8 |
| | Hz, 1H), 2.35 (s, 3H), 0.96 (s, 9H), 1H not observed (NH); LC/MS (ESI): |
| | 464.2 [M + H]⁺ |
| 296 | LC/MS (ESI): 450.2 [M + H]⁺ |
| 297 | LC/MS (ESI): 434.1 [M + H]⁺ |
| 298 | ¹H NMR (DMSO-d₆, 400 MHz) δ = 8.46 (d, J = 9.9 Hz, 1H), 7.88 (s, 1H), 7.81 |
| | (d, J = 10.0 Hz, 1H), 7.47 (d, J = 10.0 Hz, 1H), 5.25 (s, 2H), 4.35 (br d, J = |
| | 11.3 Hz, 1H), 4.16 (s, 3H), 4.11 (br d, J = 12.0 Hz, 1H), 3.05 (br d, J = 11.4 |
| | Hz, 1H), 2.66-2.84 (m, 2H), 2.53-2.61 (m, 1H), 2.49 (br s, 3H), 2.32 (br dd, J = |
| | 10.9, 1.8 Hz, 1H), 0.96 (s, 9H) ; 1H not observed (NH); LC/MS (ESI): 465.2 |
| | [M + H]⁺ |
| 299 | LC/MS (ESI): 467.2 [M + H]⁺ |
| 300 | LC/MS (ESI): 451.1 [M + H]⁺ |
| 301 | LC/MS (ESI): 437.2 [M + H]⁺ |
| 302 | LC/MS (ESI): 451.2 [M + H]⁺ |
| 308 | LC/MS (ESI): 447.2 [M + H]⁺ |
| 309 | LC/MS (ESI): 433.2 [M + H]⁺ |
| 310 | LC/MS (ESI): 434.3 [M + H]⁺ |
| 311 | LC/MS (ESI): 475.3 [M + H]⁺ |
| 312 | ¹H NMR (DMSO-d₆, 400 MHz) δ = 8.72 (br d, J = 6.3 Hz, 2H), 8.51-8.63 (m, |
| | 2H), 7.90 (br d, J = 3.9 Hz, 2H), 7.30 (br d, J = 9.6 Hz, 1H), 5.54-5.69 (m, 1H), |
| | 5.26 (s, 2H), 4.03 (s, 3H), 2.07 (br d, J = 8.3 Hz, 2H), 1.23 (m, 8H), 1.07-1.14 |
| | (s, 6H), 1H not observed (NH); LC/MS (ESI): 475.3 [M + H]⁺ |
| 313 | LC/MS (ESI): 447.24 [M + H]⁺ |
| 314 | LC/MS (ESI): 483.4 [M + H]⁺ |
| 315 | LC/MS (ESI): 515.25 [M + H]⁺ |
| 316 | LC/MS (ESI): 444.2 [M + H]⁺ |
| 317 | LC/MS (ESI): 444.3 [M + H]⁺ |
| 318 | LC/MS (ESI): 447.3 [M + H]⁺ |
| 319 | LC/MS (ESI): 474.3 [M + H]⁺ |
| 320 | LC/MS (ESI): 451.5 [M + H]⁺ |
| 321 | LC/MS (ESI): 465.3 [M + H]⁺ |
| 322 | LC/MS (ESI): 474.2 [M + H]⁺ |
| 323 | LC/MS (ESI): 484.3 [M + H]⁺ |
| 324 | ¹H NMR (DMSO-d₆, 400 MHz) δ = 8.77 (d, J = 9.6 Hz, 1H), 8.69 (d, J = 4.9 |
| | Hz, 2H), 8.17-8.23 (m, 3H), 7.90 (d, J = 8.0 Hz, 1H), 7.85 (d, J = 5.0 Hz, 2H), |
| | 7.38 (d, J = 9.6 Hz, 1H), 5.62-5.70 (m, 1H), 5.25 (s, 2H), 2.27 (br dd, J = 13.0, |
| | 3.9 Hz, 2H), 1.66 (br t, J = 11.8Hz, 2H), 1.46 (s, 6H), 1.37-1.43 (m, 6H); |
| | LC/MS (ESI): 444.3 [M + H]⁺ |
| 325 | LC/MS (ESI): 512.2 [M + H]⁺ |
| 326 | ¹LC/MS (ESI): 512.3 [M + H]⁺ |
| 327 | LC/MS (ESI): 445.3 [M + H]⁺ |
| 328 | LC/MS (ESI): 512.3 [M + H]⁺ |
| 329 | LC/MS (ESI): 505.2 [M + H]⁺ |
| 330 | LC/MS (ESI): 445.3 [M + H]⁺ |
| 331 | LC/MS (ESI): 450.1 [M + H]⁺ |
| 332 | LC/MS (ESI): 450.1 [M + H]⁺ |
| 333 | LC/MS (ESI): 462.24 [M + H]⁺ |
| 334 | LC/MS (ESI): 475.3 [M + H]⁺ |
| 335 | LC/MS (ESI): 497.3 [M + H]⁺ |
| 336 | LC/MS (ESI): 483.2 [M + H]⁺ |
| 337 | LC/MS (ESI): 433.3 [M + H]⁺ |
| 338 | LC/MS (ESI): 447.2 [M + H]⁺ |
| 339 | LC/MS (ESI): 461.3 [M + H]⁺ |
| 340 | LC/MS (ESI): 487.3 [M + H]⁺ |
| 341 | LC/MS (ESI): 498.2 [M + H]⁺ |
| 342 | LC/MS (ESI): 461.3 [M + H]⁺ |
| 343 | LC/MS (ESI): 497.2 [M + H]⁺ |
| 344 | LC/MS (ESI): 417.2 [M + H]⁺ |
| 345 | LC/MS (ESI): 429.2 [M + H]⁺ |
| 346 | LC/MS (ESI): 478.2 [M + H]⁺ |
| 347 | LC/MS (ESI): 489.3 [M + H]⁺ |
| 348 | LC/MS (ESI): 502.3 [M + H]⁺ |
| 349 | ¹H NMR (DMSO-d₆, 400 MHZ) δ = 8.72 (br d, J = 10.9 Hz, 1H), 8.58 (d, J = |
| | 9.9 Hz, 1H), 8.31 (s, 1H), 8.01 (s, 1H), 7.96 (s, 1H), 7.92 (dd, J = 1.3, 7.9 Hz, |
| | 1H), 7.87-7.77 (m, 1H), 7.69 (d, J = 7.9 Hz, 1H), 7.46 (d, J = 10.0 Hz, 1H), |
| | 5.12 (s, 2H), 5.11-5.02 (m, 1H), 3.88 (s, 3H), 2.94 (s, 3H), 1.95-1.85 (m, |
| | 2H), 1.83-1.76 (m, 2H), 1.52 (s, 6H), 1.42 (s, 6H); LC/MS (ESI): 460.2 |
| | [M + H]⁺ |
| 350 | LC/MS (ESI): 483.2 [M + H]⁺ |
| 351 | LC/MS (ESI): 486.3 [M + H]⁺ |
| 352 | LC/MS (ESI): 445.2 [M + H]⁺ |
| 353 | LC/MS (ESI): 431.1 [M + H]⁺ |
| 354 | ¹H NMR (400 MHZ, DMSO-d₆) δ = 8.84-8.69 (m, 1H), 8.62-8.56 (m, 1H), |
| | 8.23 (s, 2H), 8.03 (s, 1H), 7.98 (dd, J = 1.4, 7.9 Hz, 1H), 7.93-7.84 (m, 1H), |
| | 7.69 (d, J = 8.0 Hz, 1H), 7.54-7.42 (m, 1H), 5.12 (br d, J = 1.3 Hz, 2H), 5.10 - |
| | 5.00 (m, 1H), 2.95 (s, 3H), 1.96-1.87 (m, 2H), 1.83-1.77 (m, 2H), 1.52 (s, |
| | 6H), 1.42 (s, 6H); LC/MS (ESI): 446.2 [M + H]⁺ |
| 355 | LC/MS (ESI): 475.2 [M + H]⁺ |
| 356 | ¹H NMR (DMSO-d₆, 400 MHz) δ = 8.74 (d, J = 9.2 Hz, 2H), 8.27-8.21 (m, |
| | 2H), 7.81 (d, J = 7.8 Hz, 1H), 7.31 (d, J = 9.5 Hz, 1H), 5.67-5.59 (m, 1H), |
| | 5.21 (s, 2H), 4.12 (s, 3H), 2.16-2.07 (m, 2H), 1.35-1.30 (m, 2H), 1.28-1.24 |
| | (m, 6H), 1.15 (br s, 6H), 1H not observed (NH); LC/MS (ESI): 448.2 [M + H]⁺ |
| 357 | LC/MS (ESI): 473.3 [M + H]⁺ |
| 358 | LC/MS (ESI): 449.3 [M + H]⁺ |
| 359 | LC/MS (ESI): 515.2 [M + H]⁺ |
| 360 | LC/MS (ESI): 489.2 [M + H]⁺ |
| 361 | LC/MS (ESI): 416.3 [M + H]⁺ |
| 362 | ¹HNMR (DMSO-d₆, 400 MHz) δ = 9.26 (br d, J = 10.5 Hz, 1H), 8.76 (d, J = |
| | 9.6 Hz, 1H), 8.42 (s, 2H), 8.24 (s, 1H), 8.20 (d, J = 7.9 Hz, 1H), 7.82 (d, J = |
| | 7.9 Hz, 1H), 7.37 (d, J = 9.6 Hz, 1H), 5.71-5.60 (m, 1H), 5.20 (s, 2H), 4.23 |
| | (s, 3H), 2.31 (br dd, J = 3.1, 13.1 Hz, 2H), 1.80 (br t, J = 11.9 Hz, 2H), 1.52 |
| | (m, 12H); LC/MS (ESI): 448.2 [M + H]⁺ |
| 363 | LC/MS (ESI): 528.2 [M + H]⁺ |
| 364 | LC/MS (ESI): 504.3 [M + H]⁺ |
| 365 | LC/MS (ESI): 491.3 [M + H]⁺ |
| 366 | LC/MS (ESI): 430.2 [M + H]⁺ |
| 367 | ¹H NMR (DMSO-d₆, 400 MHz) δ = 10.02 (br dd, J = 3.4, 2.2 Hz, 1H), 9.07 (br |
| | d, J = 9.0 Hz, 1H), 8.71-8.79 (m, 1H), 8.32 (s, 1H), 8.01 (s, 1H), 7.92-8.00 (m, |
| | 2H), 7.71 (d, J = 8.0 Hz, 1H), 5.07 (s, 2H), 4.70-4.88 (m, 1H), 3.88 (s, 3H), |
| | 3.11 (s, 3H), 2.16-2.33 (m, 4H), 1.76-1.92 (m, 4H), 1.47 (s, 6H); LC/MS |
| | (ESI): 458.2 [M + H]⁺ |
| 368 | LC/MS (ESI): 502.3 [M + H]⁺ |
| 369 | LC/MS (ESI): 448.2 [M + H]⁺ |
| 370 | ¹H NMR (DMSO-d₆, 400 MHz) δ = 8.50 (d, J = 9.9 Hz, 1H), 8.42 (s, 1H), 8.23 |
| | (s, 1H), 8.17 (d, J = 7.5 Hz, 1H), 7.80 (d, J = 8.0 Hz, 1H), 7.30 (d, J = 10.0 Hz, |
| | 1H), 5.19 (s, 2H), 5.07-4.97 (m, 1H), 4.23 (s, 3H), 2.90 (s, 3H), 1.66-1.43 |
| | (m, 4H), 1.30 (s, 6H), 1.15 (br s, 6H), 1H not observed (NH); LC/MS (ESI): |
| | 461.2 [M + H]⁺ |
| 371 | LC/MS (ESI): 505.3 [M + H]⁺ |
| 372 | ¹H NMR (DMSO-d₆, 400 MHZ) δ = 8.55 (d, J = 9.9 Hz, 1H), 8.04 (s, 1H), |
| | 8.03-8.00 (m, 1H), 7.97-7.93 (m, 1H), 7.93-7.87 (m, 1H), 7.87-7.81 (m, 1H), |
| | 7.44 (d, J = 10.0 Hz, 1H), 5.22 (s, 2H), 5.09 (br t, J = 12.2 Hz, 1H), 4.13 (s, |
| | 3H), 2.94 (s, 3H), 1.96-1.85 (m, 2H), 1.84-1.75 (m, 2H), 1.52 (s, 6H), 1.42 |
| | (s, 6H); LC/MS (ESI): 461.3 [M + H]⁺ |
| 373 | ¹HNMR (DMSO-d₆, 400 MHz) δ 8.51 (d, J = 9.9 Hz, 1H), 8.41-8.32 (m, 2H), |
| | 7.92 (d, J = 7.9 Hz, 1H), 7.32 (br d, J = 9.6 Hz, 1H), 5.24 (s, 2H), 5.05 (br d, |
| | J = 5.8 Hz, 1H), 4.47 (s, 3H), 2.91 (s, 3H), 1.77-1.46 (m, 4H), 1.44-1.03 (m, |
| | 12H), 1H not observed (NH); LC/MS (ESI): 462.2 [M + H]⁺ |
| 374 | LC/MS (ESI): 416.2 [M + H]⁺ |
| 375 | LC/MS (ESI): 488.3 [M + H]⁺ |
| 376 | LC/MS (ESI): 498.1 [M + H]⁺ |
| 377 | LC/MS (ESI): 428.2 [M + H]⁺ |
| 378 | LC/MS (ESI): 462.3 [M + H]⁺ |
| 379 | LC/MS (ESI): 418.2 [M + H]⁺ |
| 380 | LC/MS (ESI): 402.2 [M + H]⁺ |
| 381 | LC/MS (ESI): 478.3 [M + H]⁺ |
| 382 | LC/MS (ESI): 465.3 [M + H]⁺ |
| 383 | LC/MS (ESI): 463.3 [M + H]⁺ |
| 384 | LC/MS (ESI): 457.1 [M + H]⁺ |
| 385 | LC/MS (ESI): 488.3 [M + H]⁺ |
| 386 | LC/MS (ESI): 476.3 [M + H]⁺ |
| 387 | LC/MS (ESI): 461.3 [M + H]⁺ |
| 388 | LC/MS (ESI): 400.3 [M + H]⁺ |
| 389 | LC/MS (ESI): 472.4 [M + H]⁺ |
| 390 | LC/MS (ESI): 470.4 [M + H]⁺ |
| 391 | LC/MS (ESI): 430.3 [M + H]⁺ |
| 392 | LC/MS (ESI): 518.3 [M + H]⁺ |
| 394 | LC/MS (ESI): 420.3 [M + H]⁺ |
| 395 | LC/MS (ESI): 418.2 [M + H]⁺ |
| 396 | LC/MS (ESI): 448.2 [M + H]⁺ |
| 397 | LC/MS (ESI): 476.2 [M + H]⁺ |
| 398 | LC/MS (ESI): 474.2 [M + H]⁺ |
| 399 | LC/MS (ESI): 432.2 [M + H]⁺ |
| 400 | LC/MS (ESI): 444.3 [M + H]⁺ |
| 401 | LC/MS (ESI): 456.3 [M + H]⁺ |
| 402 | LC/MS (ESI): 458.3 [M + H]⁺ |
| 403 | LC/MS (ESI): 477.3 [M + H]⁺ |
| 404 | LC/MS (ESI): 432.2 [M + H]⁺ |
| 405 | LC/MS (ESI): 444.2 [M + H]⁺ |
| 406 | LC/MS (ESI): 487.3 [M + H]⁺ |
| 407 | ¹H NMR (DMSO-d₆, 400 MHz) δ = 8.51 (d, J = 9.9 Hz, 1H), 8.31 (s, 1H), 8.00 |
| | (s, 1H), 7.96-7.89 (m, 2H), 7.70 (d, J = 7.9 Hz, 1H), 7.49 (d, J = 10.0 Hz, |
| | 1H), 5.11 (s, 2H), 4.33-4.23 (m, 1H), 4.17-4.08 (m, 1H), 3.88 (s, 3H), 3.13- |
| | 3.05 (m, 1H), 2.99-2.90 (m, 1H), 2.84-2.77 (m, 1H), 2.77-2.71 (m, 1H), |
| | 2.14-1.97 (m, 1H), 0.87-0.77 (m, 1H), 0.52-0.43 (m, 2H), 0.32 (br s, 2H), |
| | 1H not observed (NH); LC/MS (ESI): 416.2 [M + H]⁺ |
| 408 | LC/MS (ESI): 448.3 [M + H]⁺ |
| 409 | LC/MS (ESI): 446.3 [M + H]⁺ |
| 410 | LC/MS (ESI): 490.3 [M + H]⁺ |
| 411 | LC/MS (ESI): 476.3 [M + H]⁺ |
| 412 | LC/MS (ESI): 416.3 [M + H]⁺ |
| 413 | LC/MS (ESI): 477.2 [M + H]⁺ |
| 414 | LC/MS (ESI): 476.3 [M + H]⁺ |
| 415 | LC/MS (ESI): 476.2 [M + H]⁺ |
| 416 | ¹H NMR (DMSO-d₆, 400 MHz) δ = 8.73 (d, J = 9.6 Hz, 1H), 8.20 (br d, J = |
| | 4.5 Hz, 1H), 8.06 (s, 1H), 7.81-7.77 (m, 2H), 7.76-7.73 (m, 1H), 7.33 (d, J = |
| | 9.5 Hz, 1H), 5.49 (s, 1H), 5.16 (s, 2H), 3.81 (s, 3H), 2.34 (s, 3H), 2.33-2.29 |
| | (m, 2H), 2.03-1.93 (m, 2H), 1.72 (br d, J = 2.5 Hz, 2H), 1.66-1.56 (m, 2H), |
| | 1.34 (br s, 6H); LC/MS (ESI): 459.3 [M + H] + |
| 417 | ¹H NMR (DMSO-d₆, 400 MHZ) δ = 9.40 (s, 1H), 8.03 (s, 1H), 7.68-7.81 (m, |
| | 3H), 5.11-5.18 (m, 1H), 5.09 (s, 2H), 3.80 (s, 3H), 3.07 (s, 3H), 2.33 (s, 3H), |
| | 1.86-1.97 (m, 2H), 1.75-1.85 (m, 2H), 1.49 (s, 6H), 1.41 (s, 6H), 1H not |
| | observed (NH); LC/MS (ESI): 475.3 [M + H]⁺ |
| 418 | LC/MS (ESI): 445.2 [M + H]⁺ |
| 419 | LC/MS (ESI): 445.2 [M + H]⁺ |
| 420 | LC/MS (ESI): 462.2 [M + H]⁺ |
| 421 | LC/MS (ESI): 461.3 [M + H]⁺ |
| 422 | LC/MS (ESI): 477.2 [M + H]⁺ |
| 423 | ¹H NMR (DMSO-d₆, 400 MHz) δ = 8.48 (d, J = 9.9 Hz, 1H), 8.00 (s, 1H), |
| | 7.82-7.72 (m, 3H), 7.37-7.30 (m, 1H), 5.13 (s, 2H), 5.11-5.02 (m, 1H), 3.82 (s, |
| | 3H), 2.91 (s, 3H), 2.35 (s, 3H), 2.15 (br d, J = 8.1 Hz, 2H), 2.00-1.87 (m, |
| | 4H), 1.79 (br dd, J = 5.4, 13.4 Hz, 2H), 1.44 (s, 6H), 1H not observed (NH); |
| | LC/MS (ESI): 472.2 [M + H]⁺ |
| 424 | LC/MS (ESI): 474.2 [M + H]⁺ |
| 425 | ¹H NMR (400 MHZ, DMSO-d₆) δ = 8.86 (br s, 1H), 8.72-8.83 (m, 2H), 8.43 (s, |
| | 1H), 8.26 (s, 1H), 8.22 (dd, J = 7.9, 1.3 Hz, 1H), 7.84 (d, J = 8.0 Hz, 1H), 7.38 |
| | (d, J = 9.6 Hz, 1H), 5.49-5.58 (m, 1H), 5.20 (s, 2H), 4.23 (s, 3H), 2.45 (br d, J |
| | = 5.9 Hz, 2H), 2.13-2.21 (m, 2H), 1.89 (br d, J = 8.3 Hz, 2H), 1.79 (br t, J = |
| | 12.1 Hz, 2H), 1.45 (s, 6H), 1H not observed (NH); LC/MS (ESI): 446.2 |
| | [M + H]⁺ |
| 426 | LC/MS (ESI): 446.2 [M + H]⁺ |
| 427 | ¹HNMR (DMSO-d₆, 400 MHz) δ = 9.41 (s, 1H), 8.04 (s, 1H), 7.74-7.80 (m, |
| | 3H), 5.11-5.17 (m, 1H), 5.10 (s, 2H), 3.81 (s, 3H), 3.06 (s, 3H), 2.34 (s, 3H), |
| | 2.08-2.16 (m, 2H), 1.98-2.05 (m, 2H), 1.92 (br d, J = 8.3 Hz, 2H), 1.84 (dd, J = |
| | 13.6, 5.6 Hz, 2H), 1.45 (s, 6H), 1H not observed (NH); LC/MS (ESI): 473.3 |
| | [M + H]⁺ |
| 428 | LC/MS (ESI): 459.2 [M + H]⁺ |
| 429 | LC/MS (ESI): 460.2 [M + H]⁺ |
| 430 | LC/MS (ESI): 491.2 [M + H]⁺ |
| 431 | LC/MS (ESI): 492.2 [M + H]⁺ |
| 432 | ¹HNMR (DMSO-d₆, 400 MHz) δ = 8.51 (d, J = 9.9 Hz, 1H), 7.84-7.73 (m, |
| | 3H), 7.38 (d, J = 10.0 Hz, 1H), 5.17 (s, 2H), 5.12-5.00 (m, 1H), 2.92 (s, 3H), |
| | 2.65 (s, 3H), 2.44-2.39 (m, 3H), 2.25-2.12 (m, 2H), 2.01-1.87 (m, 4H), |
| | 1.80 (br dd, J = 5.4, 13.3 Hz, 2H), 1.43 (s, 6H), 1H not observed (NH); LC/MS |
| | (ESI): 489.1 [M + H]⁺ |
| 433 | LC/MS (ESI): 479.2 [M + H]⁺ |
| 434 | LC/MS (ESI): 476.1 [M + H]⁺ |
| 435 | LC/MS (ESI): 508.3 [M + H]⁺ |
| 436 | LC/MS (ESI): 489.2 [M + H]⁺ |
| 437 | LC/MS (ESI): 490.2 [M + H]⁺ |
| 438 | LC/MS (ESI): 459.2 [M + H]⁺ |
| 439 | ¹H NMR (DMSO-d₆, 400 MHz) δ = 9.26 (br d, J = 9.3 Hz, 1H), 9.11 (br d, J = |
| | 8.9 Hz, 1H), 8.67 (d, J = 9.6 Hz, 1H), 7.93-7.99 (m, 2H), 7.76 (d, J = 8.0 Hz, |
| | 1H), 7.29 (d, J = 9.5 Hz, 1H), 5.38-5.48 (m, 1H), 5.12 (s, 2H), 4.07 (s, 3H), |
| | 2.38-2.40 (m, 3H), 2.34 (br dd, J = 13.1, 5.8 Hz, 2H), 2.06 (br d, J = 8.0 Hz, |
| | 2H), 1.72-1.89 (m, 4H), 1.39 (s, 6H); |
| | LC/MS (ESI): 460.2 [M + H]⁺ |
| 441 | LC/MS (ESI): 491.2 [M + H]⁺ |
| 442 | LC/MS (ESI): 465.2 [M + H]⁺ |
| 443 | LC/MS (ESI): 497.2 [M + H]⁺ |
| 444 | LC/MS (ESI): 476.1 [M + H]⁺ |
| 445 | LC/MS (ESI): 506.3 [M + H]⁺ |
| 446 | ¹H NMR (400 MHZ, DMSO-d₆) δ = 8.70 (d, J = 9.7 Hz, 1H), 8.29 (br d, J = |
| | 2.9 Hz, 1H), 8.26 (s, 1H), 8.21 (s, 1H), 8.09 (s, 1H), 7.35 (d, J = 9.6 Hz, 1H), |
| | 5.52-5.44 (m, 1H), 5.35 (s, 2H), 3.82 (s, 3H), 2.38 (s, 3H), 2.27-2.18 (m, |
| | 2H), 1.88 (br s, 2H), 1.67-1.60 (m, 2H), 1.60-1.46 (m, 2H), 1.26 (br s, 6H); |
| | LC/MS (ESI): 484.2 [M + H]⁺ |
| 447 | LC/MS (ESI): 493.3 [M + H]⁺ |
| 448 | LC/MS (ESI): 460.2 [M + H]⁺ |
| 449 | LC/MS (ESI): 457.3 [M + H]⁺ |
| 451 | LC/MS (ESI): 465.2 [M + H]⁺ |
| 452 | ¹H NMR (400 MHZ, DMSO-d₆) δ = 9.10 (s, 1H), 8.49 (s, 2H), 8.30 (s, 1H), |
| | 7.35 (d, J = 10.0 Hz, 1H), 5.16 (s, 2H), 5.02 (td, J = 5.6, 11.3 Hz, 1H), 3.85 (s, |
| | 3H), 2.91 (s, 3H), 2.52 (br s, 3H), 1.98 (br d, J = 7.6 Hz, 2H), 1.79 (br t, J = |
| | 10.9 Hz, 2H), 1.74-1.68 (m, 2H), 1.66 (br d, J = 5.1 Hz, 2H), 1.31 (s, 6H); |
| | LC/MS (ESI): 474.3 [M + H]⁺ |
| 453 | 1H NMR (400 MHZ, DMSO-d₆) δ = 9.34 (s, 1H), 8.71 (d, J = 9.6 Hz, 1H), 7.35 |
| | (d, J = 9.6 Hz, 1H), 5.52-5.42 (m, 1H), 5.25 (s, 2H), 4.23 (s, 3H), 2.62 (s, |
| | 3H), 2.17 (br dd, J = 5.8, 11.9 Hz, 2H), 1.80 (br d, J = 6.6 Hz, 2H), 1.53 (br d, |
| | J = 6.9 Hz, 2H), 1.46-1.37 (m, 2H), 1.21 (s, 6H), 1H not observed (NH); |
| | LC/MS (ESI): 462.2 [M + H]⁺ |
| 454 | LC/MS (ESI): 431.2 [M + H]⁺ |
| 455 | LC/MS (ESI): 461.3 [M + H]⁺ |
| 456 | ¹H NMR (400 MHZ, DMSO-d₆) δ = 13.17-12.36 (m, 1H), 8.99 (s, 1H), 8.71 |
| | (d, J = 9.5 Hz, 1H), 8.25 (s, 1H), 8.20 (br s, 1H), 7.96 (s, 1H), 7.35 (d, J = 9.6 |
| | Hz, 1H), 5.55-5.45 (m, 1H), 5.24 (s, 2H), 2.56 (s, 3H), 2.28 (br dd, J = 5.9, |
| | 12.5 Hz, 2H), 1.96 (br d, J = 7.8 Hz, 2H), 1.69 (br d, J = 7.5 Hz, 2H), 1.59 (br |
| | t, J = 11.6 Hz, 2H), 1.32 (s, 6H); LC/MS (ESI): 446.2 [M + H]⁺ |
| 457 | ¹H NMR (400 MHZ, DMSO-d₆) δ = 9.02 (s, 1H), 8.70 (d, J = 9.5 Hz, 1H), 8.35 |
| | (s, 1H), 7.92 (d, J = 0.8 Hz, 1H), 7.36 (d, J = 9.6 Hz, 1H), 5.48 (td, J = 4.2, 8.1 |
| | Hz, 1H), 5.26 (s, 2H), 3.84 (s, 3H), 2.48 (s, 3H), 2.38-2.19 (m, 3H), 2.05- |
| | 1.87 (m, 3H), 1.53 (s, 3H), 1.49 (s, 3H), 1.46-1.45 (m, 6H), NH not observed; |
| | LC/MS (ESI): 238.6 [M + H]⁺ |
| 458 | LC/MS (ESI): 481.0 [M + H]⁺ |
| 459 | LC/MS (ESI): 462.2 [M + H]⁺ |
| 460 | LC/MS (ESI): 481.2 [M + H]⁺ |
| 461 | LC/MS (ESI): 247.2 [M + H] ⁺ |
| 462 | LC/MS (ESI): 434.2 [M + H]⁺ |
| 463 | LC/MS (ESI): 448.2 [M + H]⁺ |
| 464 | LC/MS (ESI): 489.3 [M + H]⁺ |
| 465 | LC/MS (ESI): 463.3 [M + H]⁺ |
| 466 | ¹H NMR (400 MHZ, C₂DF₃O₂) δ = 9.33 (s, 1H), 8.74 (s, 1H), 8.69 (s, 1H), |
| | 8.62 (s, 1H), 7.05-6.51 (m, 1H), 5.43 (s, 2H), 4.70-4.54 (m, 1H), 4.23 (s, |
| | 3H), 4.13 (br t, J = 7.6 Hz, 2H), 3.46 (br t, J = 6.8 Hz, 2H), 2.72 (s, 3H), 2.33- |
| | 2.14 (m, 4H), 1.67 (s, 6H), 1.55 (s, 6H); |
| | LC/MS (ESI): 487.3 [M + H]⁺ |
| 467 | LC/MS (ESI): 477.2 [M + H]⁺ |
| 468 | LC/MS (ESI): 503.2 [M + H]⁺ |
| 469 | LC/MS (ESI): 231.6 [M + H]⁺ |
| 470 | LC/MS (ESI): 476.2 [M + H]⁺ |
| 471 | LC/MS (ESI): 248.6 [M + H]⁺ |
| 472 | LC/MS (ESI): 240.5 [M + H]⁺ |
| 473 | ¹H NMR (400 MHZ, DMSO-d₆) δ = 8.98 (s, 1H), 8.73 (d, J = 9.6 Hz, 1H), 8.38 |
| | (s, 1H), 8.16 (br s, 2H), 7.89 (s, 1H), 7.37 (d, J = 9.5 Hz, 1H), 5.49 (br s, 1H), |
| | 5.24 (s, 2H), 3.82 (s, 3H), 2.48 (s, 3H), 2.29-2.16 (m, 3H), 2.03-1.86 (m, |
| | 3H), 1.52 (s, 3H), 1.49-1.37 (m, 9H); LC/MS (ESI): 238.7 [M + 2H]2 + |
| 474 | ¹H NMR (400 MHZ, DMSO-d₆) δ = 8.98 (s, 1H), 8.76-8.69 (m, 1H), 8.38 (s, |
| | 1H), 8.25-8.06 (m, 2H), 7.89 (s, 1H), 7.37 (d, J = 9.6 Hz, 1H), 5.49 (br s, |
| | 1H), 5.24 (s, 2H), 3.82 (s, 3H), 2.48 (s, 3H), 2.30-2.16 (m, 3H), 2.03-1.87 |
| | (m, 3H), 1.52 (s, 3H), 1.49-1.40 (m, 9H); LC/MS (ESI): 238.7 [M + 2H]²⁺ |
| 475 | LC/MS (ESI): 499.1 [M + H]⁺ |
| 476 | LC/MS (ESI): 485.27 [M + H]⁺ |
| 477 | LC/MS (ESI): 480.2 [M + H]⁺ |
| 478 | LC/MS (ESI): 431.3 [M + H]⁺ |
| 479 | LC/MS (ESI): 238.6 [M + H]⁺ |
| 480 | LC/MS (ESI): 481.2 [M + H]⁺ |
| 481 | LC/MS (ESI): 462.2 [M + H]⁺ |
| 482 | LC/MS (ESI): 247.2 [M + H]⁺ |
| 483 | LC/MS (ESI): 489.4 [M + H]⁺ |
| 484 | LC/MS (ESI): 526.2 [M + H]⁺ |
| 485 | LC/MS (ESI): 463.3 [M + H]⁺ |
| 486 | LC/MS (ESI): 448.3 [M + H]⁺ |
| 487 | ¹H NMR (400 MHZ, DMSO-d₆) δ = 8.94 (d, J = 2.1 Hz, 1H), 8.56 (d, J = 9.9 |
| | Hz, 1H), 8.17 (d, J = 2.1 Hz, 1H), 8.14 (s, 1H), 7.64 (d, J = 9.9 Hz, 1H), 5.17 |
| | (s, 2H), 4.89-4.79 (m, 1H), 3.83 (s, 3H), 2.63-2.58 (m, 1H), 2.37 (s, 3H), |
| | 2.25 (br t, J = 12.9 Hz, 2H), 1.96 (br dd, J = 2.6, 13.1 Hz, 2H), 1.49 (s, 6H), |
| | 1.41 (s, 6H), 1.08-0.99 (m, 2H), 0.71-0.57 (m, 2H), NH not observed; |
| | LC/MS (ESI): 501.1 [M + H]⁺ |
| 488 | LC/MS (ESI): 249.1 [M + H]⁺ |
| 489 | LC/MS (ESI): 231.6 [M + H]⁺ |
| 490 | LC/MS (ESI): 476.2 [M + H]⁺ |
| 491 | LC/MS (ESI): 480.0 [M + H]⁺ |
| 492 | LC/MS (ESI): 248.6 [M + H]⁺ |
| 493 | LC/MS (ESI): 483.2 [M + H]⁺ |
| 494 | LC/MS (ESI): 487.3 [M + H]⁺ |
| 495 | LC/MS (ESI): 449.2 [M + H]⁺ |
| 496 | ¹H NMR (400 MHZ, DMSO-d₆) δ = 9.10 (s, 1H), 8.49 (s, 2H), 8.30 (s, 1H), |
| | 7.35 (d, J = 10.0 Hz, 1H), 5.16 (s, 2H), 5.02 (td, J = 5.6, 11.3 Hz, 1H), 3.85 (s, |
| | 3H), 2.91 (s, 3H), 2.52 (br s, 3H), 1.98 (br d, J = 7.6 Hz, 2H), 1.79 (br t, J = |
| | 10.9 Hz, 2H), 1.74-1.68 (m, 2H), 1.66 (br d, J = 5.1 Hz, 2H), 1.31 (s, 6H); |
| | LC/MS (ESI): 474.3 [M + H]⁺ |
| 497 | ¹HNMR (400MHZ, METHANOL-d₄) δ = 8.96 (s, 1H), 8.78 (d, J = 9.6 Hz, |
| | 1H), 8.45 (s, 1H), 8.21 (s, 1H), 7.17 (d, J = 9.6 Hz, 1H), 5.71-5.59 (m, 1H), |
| | 5.14 (s, 2H), 3.82 (s, 3H), 2.51 (s, 3H), 2.26 (br dd, J = 2.4, 12.3 Hz, 2H), 1.53 |
| | (br t, J = 11.3 Hz, 2H), 1.40 (s, 6H), 1.31 (br s, 6H); |
| | LC/MS (ESI): 463.2 [M + H]⁺ |
| 498 | ¹H NMR (DMSO-d₆) δ = 9.38 (br s, 1H), 8.75 (br d, J = 9.3 Hz, 1H), 8.47 (br |
| | s, 1H), 7.41 (br d, J = 9.4 Hz, 1H), 5.54 (m, 1H), 5.25 (br s, 2H), 4.31 (br s, |
| | 3H), 2.46 (br s, 2H), 2.17 (br d, J = 7.6 Hz, 2H), 1.74-1.99 (m, 4H), 1.46 (br s, |
| | 6H), 1H not observed (NH); LC/MS (ESI): 448.3 [M + H]⁺ |
| 499 | LC/MS (ESI): 476.3 [M + H]⁺ |
| 500 | LC/MS (ESI): 476.2 [M + H]⁺ |
| 501 | LC/MS (ESI): 392.1 [M + H]⁺ |
| 502 | ¹H NMR (400 MHZ, DMSO-d₆) δ = 9.26 (s, 1H), 8.80-8.88 (m, 1H), 8.77 (d, J = |
| | 9.6 Hz, 2H), 8.62 (s, 1H), 7.42 (d, J = 9.6 Hz, 1H), 5.48-5.60 (m, 1H), 5.21 |
| | (s, 2H), 3.93 (s, 3H), 2.16-2.21 (m, 2H), 1.75-1.94 (m, 6H), 1.45 (s, 6H); |
| | LC/MS (ESI): 481.2 [M + H]⁺ |
| 503 | ¹H NMR (DMSO-d₆) δ = 9.35 (s, 1H), 8.91-8.99 (m, 1H), 8.84 (br d, J = 8.8 |
| | Hz, 1H), 8.77 (d, J = 9.5 Hz, 1H), 7.44 (d, J = 9.6 Hz, 1H), 5.55 (m, 1H), 5.26 |
| | (s, 2H), 4.31 (s, 3H), 2.46 (br d, J = 6.0 Hz, 2H), 2.17 (br d, J = 8.0 Hz, 2H), |
| | 1.90 (br d, J = 8.3 Hz, 2H), 1.81 (br t, J = 12.1 Hz, 2H), 1.46 (s, 6H); |
| | LC/MS (ESI): 482.2 [M + H]⁺ |
| 504 | LC/MS (ESI): 239.2 [M + H]⁺ |
| 505 | LC/MS (ESI): 249.0 [M + H]⁺ |
| 506 | LC/MS (ESI): 232.0 [M + H]⁺ |
| 507 | LC/MS (ESI): 247.5 [M + H]⁺ |
| 508 | LC/MS (ESI): 241.1 [M + 2H]²⁺ |
| 509 | LC/MS (ESI): 249.6 [M + H]⁺ |
| 510 | LC/MS (ESI): 447.2 [M + H]⁺ |
| 511 | LC/MS (ESI): 445.3 [M + H]⁺ |
| 512 | LC/MS (ESI): 433.2 [M + H]⁺ |
| 513 | LC/MS (ESI): 417.2 [M + H]⁺ |
| 514 | ¹H NMR (400 MHz, DMSO-d₆) δ: 9.02 (s, 1H), 8.47 (d, J = 9.8 Hz, 1H), 8.25 |
| | (br s, 1H), 7.87 (s, 1H), 7.22 (br d, J = 9.8 Hz, 1H), 5.23 (s, 2H), 4.87 (br s, |
| | 1H), 4.20 (br s, 1H), 3.65-3.59 (m, 1H), 3.53 (br s, 1H), 3.16 (br d, J = 10.8 |
| | Hz, 1H), 3.06 (br d, J = 10.5 Hz, 1H), 2.63 (s, 6H), 2.04 (br d, J = 10.4 Hz, |
| | 1H), 1.89 (br d, J = 10.3 Hz, 1H); LC/MS (ESI): 420.1 [M + H]⁺ |
| 515 | LC/MS (ESI): 417.1 [M + H]⁺ |
| 516 | LC/MS (ESI): 462.2 [M + H]⁺ |
| 517 | LC/MS (ESI): 431.2 [M + H]⁺ |
| 518 | LC/MS (ESI): 403.2 [M + H]⁺ |
| 519 | LC/MS (ESI): 447.2 [M + H]⁺ |
| 520 | LC/MS (ESI): 403.1 [M + H]⁺ |
| 521 | LC/MS (ESI): 434.1 [M + H]⁺ |
| 522 | LC/MS (ESI): 431.2 [M + H]⁺ |
| 523 | LC/MS (ESI): 420.1 [M + H]⁺ |
| 524 | LC/MS (ESI): 403.2 [M + H]⁺ |
| 525 | LC/MS (ESI): 431.2 [M + H]⁺ |
| 526 | LC/MS (ESI): 461.2 [M + H]⁺ |
| 527 | LC/MS (ESI): 419.2 [M + H]⁺ |
| 528 | LC/MS (ESI): 433.3 [M + H]⁺ |
| 529 | LC/MS (ESI): 403.1 [M + H]⁺ |
| 530 | LC/MS (ESI): 448.2 [M + H]⁺ |
| 531 | LC/MS (ESI): 405.2 [M + H]⁺ |
| 532 | LC/MS (ESI): 431.3 [M + H]⁺ |
| 533 | LC/MS (ESI): 419.2 [M + H]⁺ |
| 534 | LC/MS (ESI): 447.3 [M + H]⁺ |
| 535 | LC/MS (ESI): 434.0 [M + H]⁺ |
| 536 | LC/MS (ESI): 447.2 [M + H]⁺ |
| 537 | LC/MS (ESI): 420.1 [M + H]⁺ |
| 538 | LC/MS (ESI): 433.2 [M + H]⁺ |
| 539 | LC/MS (ESI): 417.2 [M + H]⁺ |
| 540 | LC/MS (ESI): 445.3 [M + H]⁺ |
| 541 | LC/MS (ESI): 448.2 [M + H]⁺ |
| 542 | LC/MS (ESI): 403.1 [M + H]⁺ |
| 543 | LC/MS (ESI): 417.2 [M + H]⁺ |
| 544 | LC/MS (ESI): 431.2 [M + H]⁺ |
| 545 | LC/MS (ESI): 447.2 [M + H]⁺ |
| 546 | LC/MS (ESI): 403.2 [M + H]⁺ |
| 547 | LC/MS (ESI): 403.1 [M + H]⁺ |
| 548 | LC/MS (ESI): 420.2 [M + H]⁺ |
| 549 | LC/MS (ESI): 464.2 [M + H]⁺ |
| 550 | LC/MS (ESI): 403.1 [M + H]⁺ |
| 551 | LC/MS (ESI): 461.3 [M + H]⁺ |
| 552 | LC/MS (ESI): 406.2 [M + H]⁺ |
| 553 | LC/MS (ESI): 431.2 [M + H]⁺ |
| 554 | LC/MS (ESI): 434.1 [M + H]⁺ |
| 555 | LC/MS (ESI): 431.2 [M + H]⁺ |
| 556 | LC/MS (ESI): 433.3 [M + H]⁺ |
| 557 | LC/MS (ESI): 419.2 [M + H]⁺ |
| 558 | LC/MS (ESI): 431.3 [M + H]⁺ |
| 559 | LC/MS (ESI): 404.2 [M + H]⁺ |
| 560 | LC/MS (ESI): 404.1 [M + H]⁺ |
| 561 | LC/MS (ESI): 405.2 [M + H]⁺ |
| 562 | LC/MS (ESI): 462.2 [M + H]⁺ |
| 563 | LC/MS (ESI): 418.2 [M + H]⁺ |
| 564 | LC/MS (ESI): 434.2 [M + H]⁺ |
| 565 | LC/MS (ESI): 432.2 [M + H]⁺ |
| 566 | LC/MS (ESI): 432.2 [M + H]⁺ |
| 567 | LC/MS (ESI): 419.3 [M + H]⁺ |
| 568 | LC/MS (ESI): 418.2 [M + H]⁺ |
| 569 | LC/MS (ESI): 447.3 [M + H]⁺ |
| 570 | LC/MS (ESI): 434.0 [M + H]⁺ |
| 571 | LC/MS (ESI): 432.2 [M + H]⁺ |
| 572 | LC/MS (ESI): 404.1 [M + H]⁺ |
| 573 | LC/MS (ESI): 406.2 [M + H]⁺ |
| 574 | LC/MS (ESI): 404.2 [M + H]⁺ |
| 575 | LC/MS (ESI): 432.2 [M + H]⁺ |
| 576 | LC/MS (ESI): 448.2 [M + H]⁺ |
| 577 | LC/MS (ESI): 422.1 [M + H]⁺ |
| 578 | LC/MS (ESI): 450.2 [M + H]⁺ |
| 579 | LC/MS (ESI): 434.2 [M + H]⁺ |
| 580 | LC/MS (ESI): 420.1 [M + H]⁺ |
| 581 | LC/MS (ESI): 450.3 [M + H]⁺ |
| 582 | LC/MS (ESI): 462.3 [M + H]⁺ |
| 583 | LC/MS (ESI): 420.2 [M + H]⁺ |
| 584 | LC/MS (ESI): 420.1 [M + H]⁺ |
| 585 | LC/MS (ESI): 448.3 [M + H]⁺ |
| 586 | LC/MS (ESI): 448.2 [M + H]⁺ |
| 587 | LC/MS (ESI): 434.2 [M + H]⁺ |
| 588 | LC/MS (ESI): 436.2 [M + H]⁺ |
| 589 | LC/MS (ESI): 494.2 [M + H]⁺ |
| 590 | LC/MS (ESI): 475.2 [M + H]⁺ |
| 591 | LC/MS (ESI): 471.2 [M + H]⁺ |
| 592 | LC/MS (ESI): 462.3 [M + H]⁺ |
| 593 | LC/MS (ESI): 474.2 [M + H]⁺ |
| 594 | LC/MS (ESI): 504.0 [M + H]⁺ |
| 595 | LC/MS (ESI): 461.5 [M + H]⁺ |
| 596 | LC/MS (ESI): 462.2 [M + H]⁺ |
| 597 | LC/MS (ESI): 474.2 [M + H]⁺ |
| 598 | LC/MS (ESI): 463.0 [M + H]⁺ |
| 599 | LC/MS (ESI): 447.2 [M + H]⁺ |
| 600 | LC/MS (ESI): 448.2 [M + H]⁺ |
| 601 | LC/MS (ESI): 461.2 [M + H]⁺ |
| 602 | LC/MS (ESI): 433.3 [M + H]⁺ |
| 603 | LC/MS (ESI): 446.3 [M + H]⁺ |
| 604 | LC/MS (ESI): 517.0 [M + H]⁺ |
| 605 | LC/MS (ESI): 249.1 [M + H]⁺ |
| 606 | LC/MS (ESI): 463.2 [M + H]⁺ |
| 607 | LC/MS (ESI): [M + H]⁺ |
| 608 | LC/MS (ESI): 482.2 [M + H]⁺ |
| 609 | LC/MS (ESI): 493.2 [M + H]⁺ |
| 610 | LC/MS (ESI): 497.1 [M + H]⁺ |
| 611 | LC/MS (ESI): 477.2 [M + H]⁺ |
| 612 | LC/MS (ESI): 461.2 [M + H]⁺ |
| 613 | LC/MS (ESI): 420.2 [M + H]⁺ |
| 614 | LC/MS (ESI): 440.2 [M + H]⁺ |
| 615 | LC/MS (ESI): 432.2 [M + H]⁺ |
| 616 | LC/MS (ESI): 462.3 [M + H]⁺ |
| 617 | LC/MS (ESI): 482.2 [M + H]⁺ |
| 618 | LC/MS (ESI): 454.1 [M + H]⁺ |
| 619 | LC/MS (ESI): 495.2 [M + H]⁺ |
| 620 | LC/MS (ESI): 452.2 [M + H]⁺ |
| 621 | LC/MS (ESI): 508.2 [M + H]⁺ |
| 622 | LC/MS (ESI): 461.2 [M + H]⁺ |
| 623 | ¹H NMR (400 MHz, DMSO-d₆) δ = 9.34 (s, 1H), 8.71 (d, J = 9.6 Hz, 1H), 7.35 |
| | (d, J = 9.6 Hz, 1H), 5.52-5.42 (m, 1H), 5.25 (s, 2H), 4.23 (s, 3H), 2.62 (s, |
| | 3H), 2.17 (br dd, J = 5.8, 11.9 Hz, 2H), 1.80 (br d, J = 6.6 Hz, 2H), 1.53 (br d, |
| | J = 6.9 Hz, 2H), 1.46-1.37 (m, 2H), 1.21 (s, 6H), NH not observed; LC/MS |
| | (ESI): 462.2 [M + H]⁺ |
| 624 | LC/MS (ESI): 464.2 [M + H]⁺ |
| 625 | LC/MS (ESI): 518.2 [M + H]⁺ |
| 626 | LC/MS (ESI): 509.3 [M + H]⁺ |
| 627 | LC/MS (ESI): 495.3 [M + H]⁺ |
| 628 | LC/MS (ESI): 496.1 [M + H]⁺ |
| 629 | LC/MS (ESI): 437.2 [M + H]⁺ |
| 630 | LC/MS (ESI): 451.2 [M + H]⁺ |
| 631 | LC/MS (ESI): 464.2 [M + H]⁺ |
| 632 | LC/MS (ESI): 534.2 [M + H]⁺ |
| 633 | LC/MS (ESI): 537.3 [M + H]⁺ |
| 634 | LC/MS (ESI): 535.2 [M + H]⁺ |
| 635 | LC/MS (ESI): 524.2 [M + H]⁺ |
| 636 | LC/MS (ESI): 457.1 [M + H]⁺ |
| 637 | LC/MS (ESI): 485.1 [M + H]⁺ |
| 638 | LC/MS (ESI): 471.1 [M + H]⁺ |
| 639 | LC/MS (ESI): 437.2 [M + H]⁺ |
| 640 | LC/MS (ESI): 451.2 [M + H]⁺ |
| 641 | LC/MS (ESI): 437.2 [M + H]⁺ |
| 642 | LC/MS (ESI): 457.1 [M + H]⁺ |
| 643 | LC/MS (ESI): 437.2 [M + H]⁺ |
| 644 | LC/MS (ESI): 455.1 [M + H]⁺ |
| 645 | LC/MS (ESI): 469.1 [M + H]⁺ |
| 646 | LC/MS (ESI): 449.2 [M + H]⁺ |
| 647 | LC/MS (ESI): 443.1 [M + H]⁺ |
| 648 | LC/MS (ESI): 465.3 [M + H]⁺ |
| 649 | LC/MS (ESI): 557.2 [M + H]⁺ |
| 650 | LC/MS (ESI): 435.2 [M + H]⁺ |
| 651 | LC/MS (ESI): 449.3 [M + H]⁺ |
| 652 | LC/MS (ESI): 469.2 [M + H]⁺ |
| 653 | LC/MS (ESI): 455.2 [M + H]⁺ |
| 654 | LC/MS (ESI): 483.2 [M + H]⁺ |
| 655 | LC/MS (ESI): 441.1 [M + H]⁺ |
| 656 | LC/MS (ESI): 485.1 [M + H]⁺ |
| 657 | LC/MS (ESI): 479.2 [M + H]⁺ |
| 658 | LC/MS (ESI): 435.2 [M + H]⁺ |
| 659 | LC/MS (ESI): 469.2 [M + H]⁺ |
| 660 | LC/MS (ESI): 507.2 [M + H]⁺ |
| 661 | ¹H NMR (400 MHZ, DMSO-d₆) δ = 9.03 (s, 1H), 8.46 (d, J = 9.8 Hz, 1H), 8.31 |
| | (s, 1H), 7.89 (s, 1H), 7.34 (d, J = 9.9 Hz, 1H), 5.25 (s, 2H), 5.05-4.93 (m, |
| | 1H), 2.91 (s, 3H), 2.64 (s, 6H), 1.96 (br d, J = 7.8 Hz, 2H), 1.80-1.72 (m, 2H), |
| | 1.68 (br d, J = 7.5 Hz, 2H), 1.63 (br dd, J = 5.4, 12.9 Hz, 2H), 1.30 (s, 6H); |
| | LC/MS (ESI): 495.2 [M + H]⁺ |
| 662 | LC/MS (ESI): 421.1 [M + H]⁺ |
| 663 | LC/MS (ESI): 463.2 [M + H]⁺ |
| 664 | LC/MS (ESI): 496.2 [M + H]⁺ |
| 665 | LC/MS (ESI): 449.21 [M + H]⁺ |
| 666 | LC/MS (ESI): 421.2 [M + H]⁺ |
| 667 | LC/MS (ESI): 471.2 [M + H]⁺ |
| 668 | LC/MS (ESI): 457.1 [M + H]⁺ |
| 669 | LC/MS (ESI): 423.2 [M + H]⁺ |
| 670 | LC/MS (ESI): 441.1 [M + H]⁺ |
| 671 | LC/MS (ESI): 469.2 [M + H]⁺ |
| 672 | LC/MS (ESI): 451.3 [M + H]⁺ |
| 673 | LC/MS (ESI): 463.2 [M + H]⁺ |
| 674 | LC/MS (ESI): 437.3 [M + H]⁺ |
| 675 | LC/MS (ESI): 479.3 [M + H]⁺ |
| 676 | LC/MS (ESI): 498.2 [M + H]⁺ |
| 677 | LC/MS (ESI): 517.4 [M + H]⁺ |
| 678 | LC/MS (ESI): 487.2 [M + H]⁺ |
| 679 | LC/MS (ESI): 527.5 [M + H]⁺ |
| 680 | LC/MS (ESI): 507.2 [M + H]⁺ |
| 681 | LC/MS (ESI): 485.2 [M + H]⁺ |
| 682 | LC/MS (ESI): 520.2 [M + H]⁺ |
| 683 | LC/MS (ESI): 505.3 [M + H]⁺ |
| 684 | LC/MS (ESI): 510.2 [M + H]⁺ |
| 685 | LC/MS (ESI): 518.2 [M + H]⁺ |
| 686 | LC/MS (ESI): 524.1 [M + H]⁺ |
| 687 | LC/MS (ESI): 503.3 [M + H]⁺ |
| 688 | LC/MS (ESI): 492.2 [M + H]⁺ |
| 689 | LC/MS (ESI): 509.3 [M + H]⁺ |
| 690 | LC/MS (ESI): 464.2 [M + H]⁺ |
| 691 | LC/MS (ESI): 476.2 [M + H]⁺ |
| 692 | LC/MS (ESI): 492.2 [M + H]⁺ |
| 693 | LC/MS (ESI): 485.2 [M + H]⁺ |
| 694 | ¹H NMR (DMSO-d₆, 400 MHz) δ 9.11 (d, 1H, J = 0.8 Hz), 8.83 (d, 1H, J = 2.0 |
| | Hz), 8.62 (d, 1H, J = 1.8 Hz), 8.48 (d, 1H, J = 10.0 Hz), 8.35 (s, 1H), 8.08 (d, 1H, |
| | J = 0.8 Hz), 7.36 (d, 1H, J = 10.0 Hz), 5.41 (s, 2H), 5.0-5.1 (m, 1H), 2.93 (s, 3H), |
| | 2.00 (br d, 2H, J = 7.5 Hz), 1.88 (br t, 2H, J = 12.4 Hz), 1.75 (br d, 2H, J = 7.8 |
| | Hz), 1.67 (br dd, 2H, J = 5.4, 12.9 Hz), 1.35 (s, 6H); LC/MS (ESI): 470.2 |
| | [M + H]⁺ |
| 695 | ¹H NMR: (400 MHZ, DMSO-d₆) δ = 8.88-8.69 (m, 2H), 8.57-8.40 (m, 2H), |
| | 7.36 (br d, J = 9.0 Hz, 1H), 5.37 (br s, 2H), 5.08 (br s, 1H), 2.94 (br s, 3H), |
| | 2.36 (br s, 3H), 2.14-1.97 (m, 4H), 1.91-1.80 (m, 2H), 1.77-1.66 (m, 2H), |
| | 1.42 (br s, 6H); LC/MS (ESI): 484.2 [M + H]⁺ |
| 696 | LC/MS (ESI): 510.3 [M + H]⁺ |

### Example 34. Decreasing MSH3 Expression

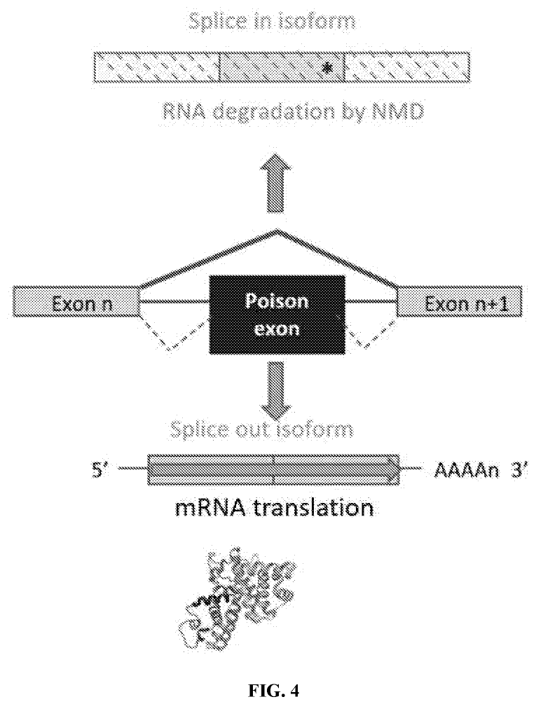

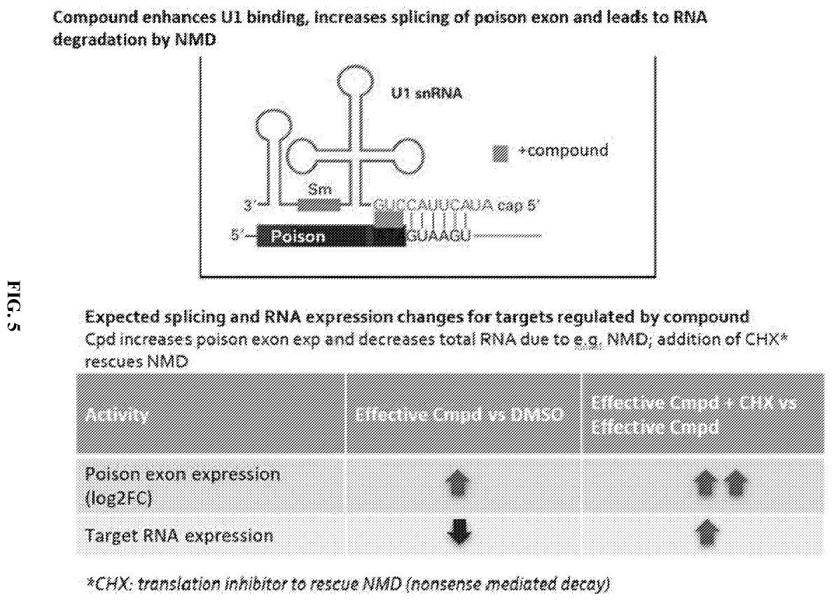

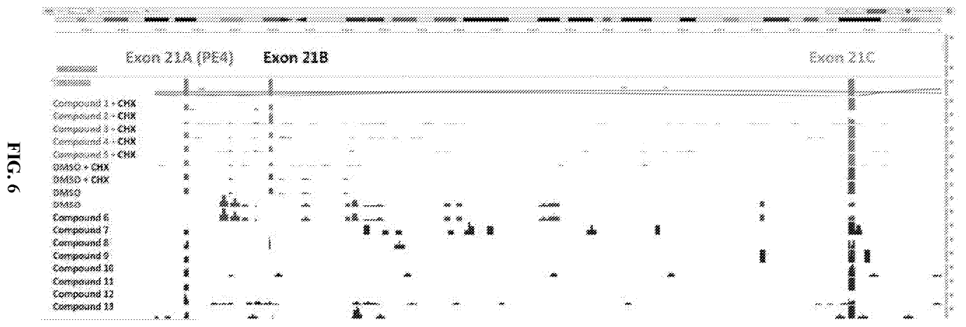

By decreasing or inhibiting expression of MSH3 gene (e.g., endogenous MSH3 gene in a cell), repair of the nucleotide repeat and the subsequent nucleotide repeat expansion in the cell can be decreased (e.g., as shown in FIG. 2), thereby treating the nucleotide repeat expansion disease or condition. As shown in FIG. 3, contacting candidate agents with MSH3 pre-mRNA or snRNAs led to decreased MSH3 expression as determined by the decrease of percentage of mRNA of MSH3. FIG. 4 and FIG. 5 illustrate exemplary mechanisms of how the candidate agents decreases MSH3 expression. Poison exon is not spliced into the mRNA under endogenous or physiological condition. However, by contacting the poison exon and one of the snRNAs with a candidate agent, the poison exon is spliced into the mRNA, where the poison exon directs the mRNA for degradation.

### Example 35. Identification and Screening for Poison Exon

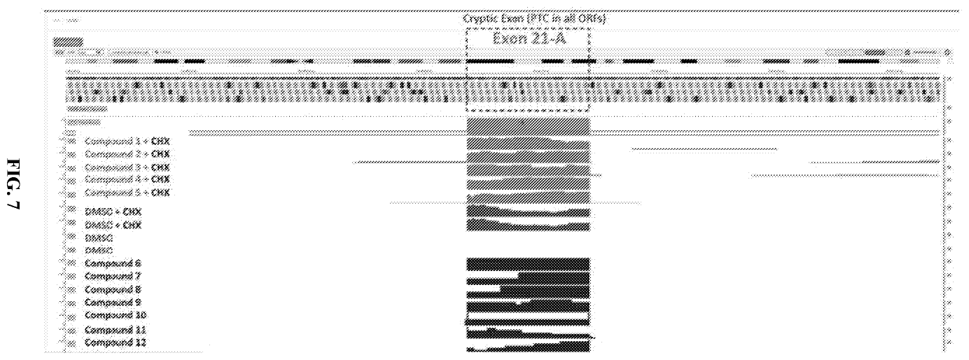

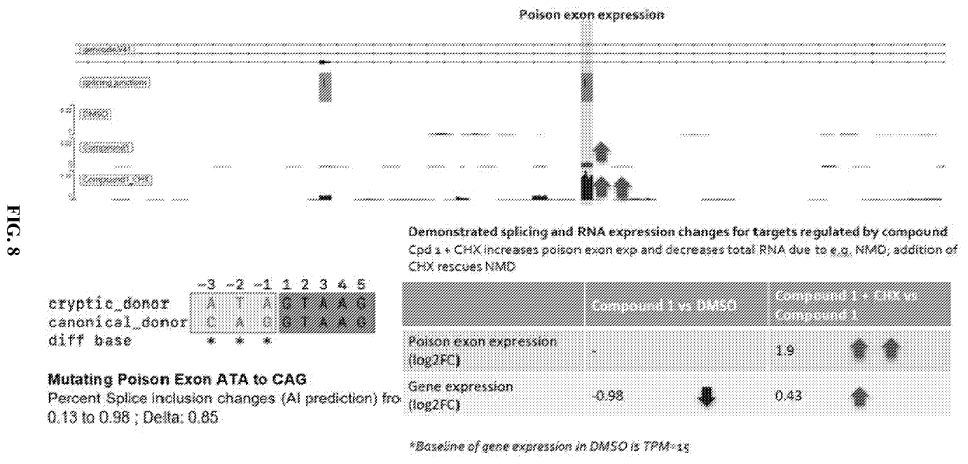

Poison exons were identified in endogenous MSH3 genome by employing a proprietary bioinformatics pipeline screening for cryptic exons in combination with examining poison exon candidates that could be regulated by an agent described herein or by nonsense mediated decay (NMD) using cycloheximide. FIG. 6 and FIG. 7 illustrate identification of Exon 21A, but not Exon 21B, as a poison exon that could be regulated by the agent described herein. FIG. 8 and FIG. 9 illustrate mutations that can be introduced into the poison exon (Exon 21A and Exon 14A_1 or Exon 14A_2, respectively), where the mutated nucleotide bases (e.g., CAG) could further enhance the suppression of the MSH3 expression. Such mutations can be utilized as a method or a system for screening additional candidate agents. FIG. 8 illustrates splicing and RNA expression changes for targets regulated by an agent described herein. Compound 1 and CHX increased poison exon expression (Exon 21A) and decreased total RNA (e.g., due to NMD). FIG. 8 also illustrates how mutating a poison exon (e.g., from ATA to CAG) increased percentage of splice inclusion of Exon 21A from 0.13 to 0.98. Such mutations can be useful for designing a method or a system for screening additional agents for modulating splicing in of poison exon into a mRNA (e.g., as shown in FIG. 10). FIG. 9 illustrates splicing and RNA expression changes for targets regulated by an agent described herein. Compound 1 and CHX increased poison exon expression (Exon 14A_1 or Exon 14A_2) and decreased total RNA (e.g., due to NMD). FIG. 9 also illustrates how mutating the poison exon (e.g., from TGA to CAG) increased percentage of splice inclusion of Exon 14A_1 from 0.00 to 0.59. Such mutations can be useful for designing a method or a system for screening additional agents for modulating splicing in of poison exon into a mRNA (e.g., as shown in FIG. 10). As shown in FIG. 10, poison exons comprising the CAG mutation were utilized to generate luciferase signal, indicating splicing in of the poison exon into an mRNA (FIG. 10C). FIG. 10A illustrates engineered minigene donor (e.g., by mutating endogenous trinucleotides to CAG as shown in FIG. 8 and FIG. 9) for driving expression of a luciferase reporter gene. FIG. 10B illustrates minigene PCR showing the presence of the poison exon comprising the CAG mutation. The wild-type (WT) mRNA did not have the poison exon as confirmed by the decreased nucleotide length. FIG. 10C illustrates increased luciferase signal generated by the presence of the minigene comprising the poison exon comprising the CAG mutation.

### Example 36. Functional Validation of MSH3 Poison Exon 14A (PE5) and Poison Exon 21A (PE4)

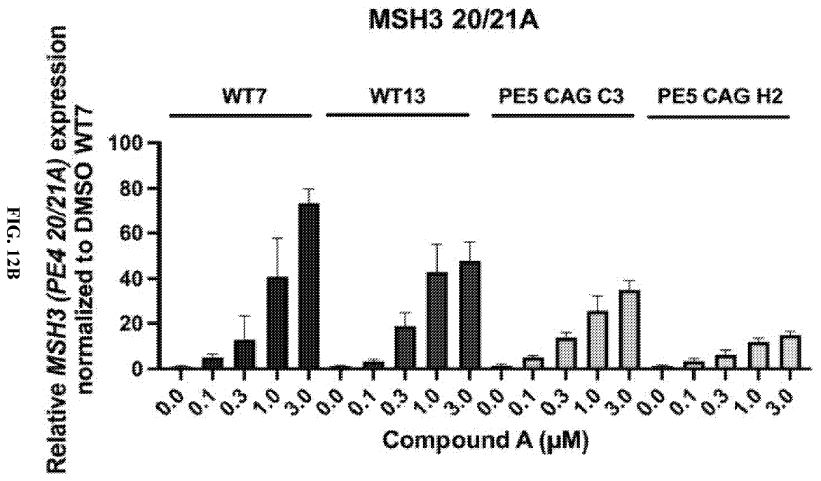

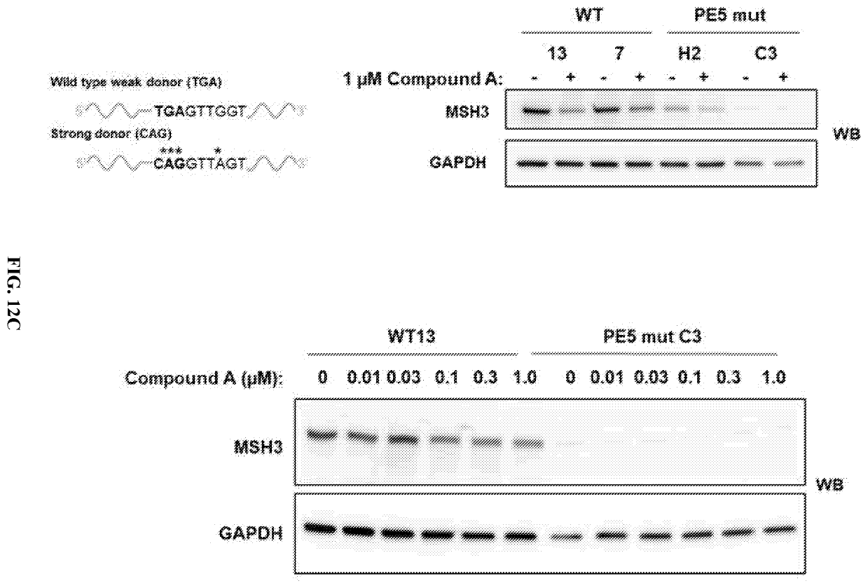

In this example, MSH3 poison exon 14A (PE5) and poison exon 21A (PE4) were validated. Briefly, HEK293 parental and clonal cells (Synthego) were cultured in DMEM (ThermoFisher Scientific) supplemented with 10% fetal bovine serum (Avantor) and 0.1 μg/mL Primocin (Invivogen). All cell lines were maintained at sub-confluency and grown in a humidified incubator at 37° C. with 5% CO₂. HEK293 clonal cells were generated by Synthego's proprietary CRISPR methods to replace PE14A 5′ splice site with a strong donor (TGAGTTG>CAGGTTA) at the endogenous locus. Two clones (C₃ and H2) were isolated to express 99-100% of the desired knock-in mutation. Two clones were generated from the parental line HEK293 without any editing (wild-type WTl3 and WT7) to be used as controls. WT control and PE14A CAG mutant clones were plated for treatment for 24 hours with an exemplary MSH3 splice modulating agent (Compound 274) as indicated in FIGS. 11A, 11B, 12A, and 12B (“Compound A”). RNA, cDNA and qPCR was prepared using TaqMan Cells-to-CT Express kit (ThermoFisher Scientific). Taqman assays (ThermoFisher Scientific) were used for quantification of total MSH3 exons 13/14 (cat #4351372, ID #: Hs00988993_ml), total MSH3 exons 20/21 (cat #4351372, ID #: Hs00989000_ml), exon 13/PE14A (Fwd: 5′-CGTAAATTGCCCGACATAGAGA (SEQ ID NO: 14); Rev: 5′-AACAGTTACGTAAGTCCTCTTGG (SEQ ID NO: 15); Probe: 5′-AGCATTTATCACAAAAAAAGTTTTAC (SEQ ID NO: 16)) and exon 20/PE21A (Fwd: 5′-CTCAGATTGGCTCCTATGTTCC (SEQ ID NO: 17); Rev: 5′-GGGCTTCTGTCCCTTCAAAT (SEQ ID NO: 18); Probe: 5′-TTCACAAGCATTTTCCTTGCA (SEQ ID NO: 19)) mRNA expression. Taqman GAPDH (cat #4448489, ID #: Hs02758991_g1) was used as a reference for data normalization. Protein lysates were prepared using RIPA buffer (ThermoFisher Scientific) supplemented with Halt protease inhibitor cocktail (ThermoFisher Scientific). Protein concentrations were determined by Rapid Gold BCA (ThermoFisher Scientific). Equal amounts of total proteins were separated on 4-15% SDS-PAGE (Bio-Rad) and blotted to PVDF membranes (Bio-Rad). Antibodies used were MSH3 (Abcam, ab275928) and GAPDH (Cell Signalling, D16H11).

FIGS. 11A and 11B demonstrate that wild-type clones responded to treatment with Compound A, lowering MSH3 expression and increasing PE5-specific expression. PE5 CAG clones C₃ and H2 had a basal level of MSH3 about 90% and 60% reduction relative to wild-type, respectively. PE5 basal expression was higher in clones, as expected. No further decrease was seen with Compound A treatment on total MSH3 13/14 levels. There was ~20-30% further increase of PE5 starting at 0.3-1 μM Compound A.

FIGS. 12A and 12B demonstrate that wild-type clones responded to treatment with Compound A, lowering MSH3 expression and increasing PE4-specific expression. MSH3 (total, exons 20/21) was about 30-50% lower in the PE5 CAG clones. FIG. 12C depicts a Western blot showing that treatment with Compound A led to a decreased in MSH3 levels in both wild-type and PE5 CAG clones.

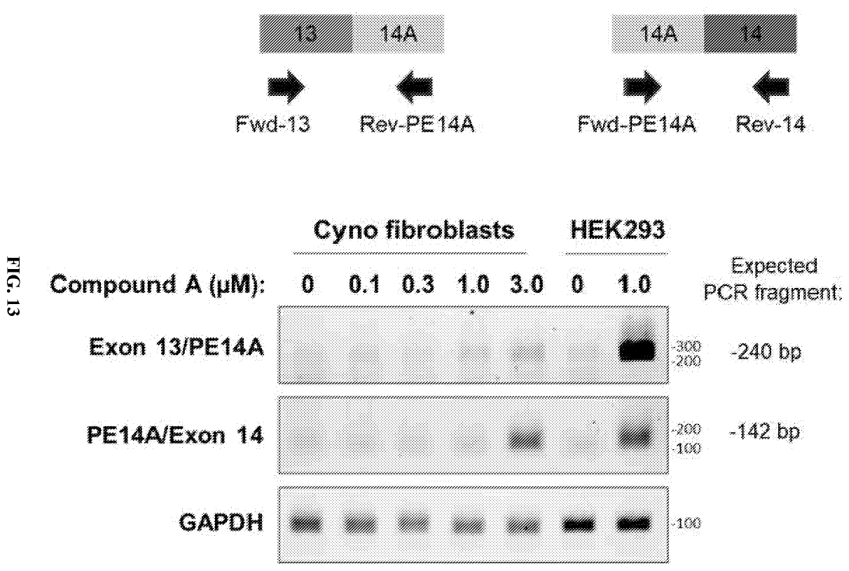

To further validate MSH3 poison exon 14A, RT-PCR was used to analyze MSH3 exon 13/poison exon 14A, and poison exon 14A/exon 14 amplicons. Briefly, Cynomolgus monkey primary dermal fibroblasts (Cellosaurus) and HEK293 cells (ATCC) were cultured in DMEM (ThermoFisher Scientific) supplemented with 10% fetal bovine serum (Avantor) and 0.1 μg/mL Primocin (Invivogen). All cell lines were maintained at sub-confluency and grown in a humidified incubator at 37° C. with 5% CO₂. Cynomolgus fibroblasts and HEK293 cells were plated for treatment for 24 hours with compound as indicated in FIG. 13. RNA was purified using Qiashredder and RNeasy kits (Qiagen), cDNA was prepared with MultiScribe Reverse Transcriptase (ThermoFisher Scientific) and PCR with GoTaq Green Master Mix (Promega). Primers (IDT) used were the following: Fwd-Exon 13 (5′-AAATAAATGCCCGGCTTGATG (SEQ ID NO: 20)), Rev-PE14A (5′-GGCTTGTGCATTGTTTTCAAATTTA (SEQ ID NO: 21)), Fwd-PE14A (5′-TAAATTTGAAAACAATGCACAAGCC (SEQ ID NO: 22)), Rev-Exon 14 (5′-AGCAGGTATTATTGCTTGAAATTCT (SEQ ID NO: 23)), Fwd-GAPDH (5′-CCACTAGGCGCTCACTGTTC (SEQ ID NO: 24)), Rev-GAPDH (5′-ACCCGTTGACTCCGACCT (SEQ ID NO: 25)).

FIG. 13 demonstrates that treatment with Compound A in both cynomolgus monkey fibroblasts and HEK293 cells led to an increase in both 13/PE14A and PE14A/14 amplicons, demonstrating that Compound A can modulate splicing of PE14A.

### Example 37. QPCR Assay for Monitoring Levels of MSH3 Upon Compound Treatment

HEK293T or AD293 cells were treated with Compound 274 at 1 μM and 10 M. RNA, cDNA and qPCR was prepared using TaqMan Cells-to-CT Express kit (ThermoFisher Scientific). Taqman assays (ThermoFisher Scientific) were used for quantification of total MSH3 exons 13/14 (cat #4351372, ID #: Hs00988993_ml), total MSH3 exons 20/21 (cat #4351372, ID #: Hs00989000_ml), exon 13/PE14A (Fwd: 5′-CGTAAATTGCCCGACATAGAGA; Rev: 5′-AACAGTTACGTAAGTCCTCTTGG; Probe: 5′-AGCATTTATCACAAAAAAAGTTTTAC) and exon 20/PE21A (Fwd: 5′-CTCAGATTGGCTCCTATGTTCC; Rev: 5′-GGGCTTCTGTCCCTTCAAAT; Probe: 5′-TTCACAAGCATTTTCCTTGCA) mRNA expression. Taqman GAPDH (cat #4448489, ID #: Hs02758991_g1) was used as a reference for data normalization. The fold-change of the MSH3 transcript at 1 μM and 10 μM of Compound 274, and the EC₅₀ of MSH3 downregulation are shown in Table 8 below.

TABLE 8

| | Fold | Fold | EC50 |
|---|---|---|---|
| Compound | change | change | nM |
| No. | (1 μM) | (10 μM) | (XtalPi) |
| 21 | 0.54 | 0.58 | |
| 51 | 0.94 | 0.26 | |
| 55 | 0.56 | 0.2 | |
| 59 | 1.01 | 0.29 | |
| 61 | 0.44 | 0.18 | |
| 62 | 0.18 | 0.08 | 689.5 |
| 69 | 0.3 | 0.11 | 885 |
| 70 | 1.39 | 0.56 | |
| 73 | 0.85 | 0.70 | |
| 74 | 0.96 | 0.33 | |
| 76 | 0.98 | 0.2 | |
| 79 | 0.92 | 0.31 | |
| 90 | 0.38 | 0.18 | |
| 91 | 0.85 | 0.74 | |
| 93 | 0.91 | 0.72 | |
| 94 | 1.06 | 0.79 | |
| 96 | 0.79 | 1.19 | |
| 98 | 0.83 | 0.25 | |
| 99 | 0.9 | 0.86 | |
| 100 | 0.65 | 0.11 | |
| 106 | 1.21 | 1.3 | |
| 107 | 0.97 | 0.75 | |
| 141 | 0.67 | 0.06 | |
| 148 | 1.03 | 0.4 | |
| 150 | 1.16 | 0.77 | |
| 156 | 0.81 | 0.19 | |
| 161 | 0.96 | 0.7 | |
| 167 | 0.47 | 0.11 | |
| 174 | 1.03 | 0.62 | |
| 175 | 0.93 | 0.7 | |
| 176 | 0.94 | 1.1 | |
| 178 | 0.1 | 0.05 | 425.4 |
| 179 | 0.93 | 0.08 | |
| 180 | 0.5 | 0.08 | 1122 |
| 181 | 0.92 | 1.03 | |
| 183 | 0.99 | 0.28 | |
| 187 | 0.92 | 0.25 | |
| 188 | 0.18 | 0.05 | 960.3 |
| 189 | 0.73 | 0.23 | |
| 190 | 1.06 | 0.45 | |
| 191 | 0.45 | 0.17 | |
| 194 | 2.24 | 0.92 | |
| 195 | 0.59 | 0.42 | |
| 200 | 0.53 | 0.53 | |
| 201 | 0.9 | 0.91 | |
| 203 | 0.56 | 0.62 | |
| 205 | 0.9 | 0.14 | 1423 |
| 206 | 0.36 | 0.13 | 506.8 |
| 207 | 1.08 | 0.18 | ~3051 |
| 211 | 0.9 | 0.55 | |
| 214 | 0.95 | 0.24 | |
| 215 | 0.97 | 0.76 | |
| 217 | 0.92 | 0.78 | |
| 226 | 0.86 | 0.58 | |
| 228 | 0.58 | 0.37 | 792.3 |
| 239 | 0.84 | 0.19 | 2035 |
| 240 | 0.87 | 0.16 | 2311 |
| 261 | 0.48 | 0.07 | |
| 274 | 0.17 | | 454.5 |
| 275 | 0.79 | 0.26 | |
| 276 | 1.03 | 0.38 | |
| 278 | 0.64 | 0.14 | |
| 280 | 0.99 | 0.66 | |
| 281 | 0.85 | 0.41 | |
| 282 | 0.3 | 0.59 | |
| 283 | 0.48 | 0.11 | 799 |
| 284 | 0.91 | 0.73 | |
| 286 | 1.17 | 0.59 | |
| 289 | 0.73 | 0.22 | |
| 291 | 0.93 | 0.81 | |
| 293 | 0.54 | 0.12 | ~1057 |
| 294 | 0.27 | 0.12 | 480.4 |
| 296 | 0.54 | | |
| 298 | 0.89 | 0.29 | |
| 362 | 0.65 | 0.75 | |
| 367 | 0.57 | 0.27 | |
| 370 | 0.69 | 0.61 | |
| 387 | 0.96 | 0.63 | |
| 399 | 0.65 | 0.43 | |
| 416 | 0.37 | | |
| 420 | 0.74 | 0.73 | |
| 423 | 0.37 | 0.1 | |
| 425 | 0.82 | 0.7 | |
| 429 | 1.02 | 0.52 | |
| 438 | 1.09 | 0.81 | |
| 439 | 0.87 | 0.72 | |
| 447 | 0.94 | | |
| 457 | 0.85 | 0.7 | |
| 458 | 0.4 | 0.11 | 820.2 |
| 460 | 0.8 | 0.25 | |
| 465 | 0.93 | 1.15 | |
| 479 | 1.19 | 0.56 | |
| 480 | 0.15 | 0.1 | 362.7 |
| 485 | 0.88 | 0.4 | |
| 487 | 1.33 | 0.31 | |
| 488 | 0.69 | 0.19 | |
| 490 | 0.9 | 0.25 | |
| 493 | 0.57 | 0.13 | |
| 496 | | | 689.5 |
| 498 | 0.67 | 0.52 | |
| 500 | 1.01 | 0.22 | |
| 503 | 0.7 | 0.18 | |
| 509 | 1.17 | 0.62 | |
| 559 | 0.93 | 0.53 | |
| 560 | 0.81 | 0.29 | |
| 563 | 1.04 | 0.35 | |
| 566 | 1.33 | 0.6 | |
| 568 | 0.77 | 0.46 | |
| 589 | 0.2 | 0.11 | 349 |
| 590 | 0.24 | 0.09 | 568.8 |
| 591 | 1.09 | 0.91 | 634 |
| 592 | 0.9 | 0.54 | |
| 593 | 1.05 | 0.34 | |
| 594 | 0.79 | 1.12 | |
| 595 | 0.91 | 0.18 | |
| 596 | 0.88 | 1.06 | |
| 597 | 0.98 | 0.29 | |
| 598 | 0.62 | 0.52 | |
| 599 | 0.94 | 0.24 | |
| 600 | 1.04 | 0.14 | |
| 601 | 1.17 | 0.82 | |
| 602 | 0.64 | 0.29 | |
| 603 | 1.55 | 0.94 | |
| 604 | 0.63 | 0.93 | |
| 605 | 0.82 | 0.56 | |
| 606 | 0.57 | 0.25 | |
| 607 | 0.72 | 0.09 | |
| 608 | 0.86 | 0.2 | |
| 609 | 0.39 | 0.07 | |
| 610 | 0.93 | 0.66 | |
| 611 | 0.9 | 0.79 | |
| 612 | 1.09 | 0.15 | |
| 613 | 1.3 | 0.91 | |
| 614 | 1.07 | 0.79 | |
| 615 | 0.81 | 0.53 | |
| 616 | 1 | 0.67 | |
| 617 | 0.93 | 0.7 | |
| 618 | 0.83 | 0.68 | |
| 619 | 0.8 | 0.1 | |
| 620 | 0.93 | 0.55 | |
| 621 | | | 229.3 |
| 622 | 0.76 | 0.30 | |
| 623 | 0.6 | 0.13 | 2993 |
| 624 | 0.6 | 0.12 | |
| 625 | 0.81 | 1.08 | |
| 626 | 0.98 | 0.28 | |
| 627 | 0.37 | 0.11 | |
| 628 | 1.01 | 0.2 | |
| 629 | 0.74 | 0.62 | |
| 630 | 1.06 | 0.72 | |
| 631 | 0.86 | 0.69 | |
| 632 | 0.72 | 0.57 | |
| 633 | 1.09 | 0.89 | |
| 634 | 0.63 | 0.76 | |
| 635 | 0.54 | 1.17 | |
| 636 | 0.95 | 0.41 | |
| 637 | 1.5 | 0.31 | |
| 638 | 1.12 | 0.22 | |
| 639 | 1.18 | 0.55 | |
| 640 | 0.99 | 0.25 | |
| 641 | 0.96 | 0.34 | |
| 642 | 0.91 | 0.21 | |
| 643 | 1.09 | 0.72 | |
| 644 | 0.87 | 0.97 | |
| 645 | 0.87 | 1.04 | |
| 646 | 1.15 | 0.76 | |
| 647 | 1.19 | 0.86 | |
| 648 | 1.13 | 0.87 | |
| 649 | 1.06 | 0.63 | |
| 650 | 0.97 | 0.75 | |
| 651 | 0.96 | 0.6 | |
| 652 | 1.03 | 0.79 | |
| 653 | 0.93 | 0.27 | |
| 654 | 0.9 | 0.61 | |
| 655 | 1.15 | 0.48 | |
| 656 | 0.7 | 0.43 | |
| 657 | 0.75 | 0.26 | |
| 658 | 1.32 | 0.51 | |
| 659 | 1.42 | 0.57 | |
| 660 | 1.01 | 0.5 | |
| 661 | 1.55 | 0.33 | |
| 662 | 1.91 | 0.87 | |
| 663 | 2.32 | 0.62 | |
| 664 | 0.94 | 0.2 | |
| 665 | 0.92 | 0.32 | |
| 666 | 0.97 | 0.59 | |
| 667 | 0.92 | 0.71 | |
| 668 | 1.03 | 0.13 | |
| 669 | 0.97 | 0.42 | |
| 670 | 0.92 | 0.44 | |
| 671 | 0.94 | 0.47 | |
| 672 | 0.95 | 0.74 | |
| 673 | 0.91 | 0.33 | |
| 674 | 0.98 | 0.49 | |
| 675 | 0.93 | 0.67 | |
| 676 | 0.48 | 0.17 | |
| 677 | 1.2 | 0.28 | |
| 678 | 1.12 | 0.19 | |
| 679 | 0.95 | 0.34 | |
| 680 | 0.94 | 0.31 | |
| 681 | 0.76 | 0.08 | |
| 682 | 0.77 | 0.17 | |
| 683 | 1.38 | 0.26 | |
| 684 | 1.33 | 0.24 | |
| 685 | 0.24 | 0.2 | |
| 686 | 1.09 | 0.26 | |
| 687 | 1.1 | 0.19 | |
| 688 | 1.23 | 0.86 | |
| 689 | 0.88 | 0.27 | |
| 690 | 0.98 | 0.65 | |
| 691 | 0.95 | 0.78 | |
| 692 | 0.97 | 0.5 | |
| 693 | 0.94 | 0.4 | |
| 694 | | | 2482 |
| 695 | | | 11063 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for modulating splicing of MSH3 pre-mRNA, the method comprising contacting a cell with an agent which modulates splicing of MSH3 pre-mRNA such that an MSH3 mRNA transcript comprising a poison exon is produced, thereby modulating splicing of MSH3 pre-mRNA.

2. A method for treating a disease or condition in a subject, the method comprising administering to the subject an agent which modulates splicing of MSH3 pre-mRNA such that an MSH3 mRNA transcript comprising a poison exon is produced in a cell of the subject, thereby treating the disease or condition.

3-30. (canceled)

31. The method of claim 2, wherein the disease or condition is selected from the group consisting of: dentatorubropallidoluysian atrophy (DRPLA), Huntington's disease (HD), spinal and bulbar muscular atrophy (SBMA), spinocerebellar ataxia (SCA) type 1, spinocerebellar ataxia (SCA) type 2, spinocerebellar ataxia (SCA) type 3, spinocerebellar ataxia (SCA) type 6, spinocerebellar ataxia (SCA) type 7, spinocerebellar ataxia (SCA) type 8, spinocerebellar ataxia (SCA) type 10, spinocerebellar ataxia (SCA) type 12, spinocerebellar ataxia (SCA) type 17, spinocerebellar ataxia (SCA) type 31, spinocerebellar ataxia (SCA) type 36, spinocerebellar ataxia (SCA) type 37, spinocerebellar ataxia (SCA) type 50, Friedreich ataxia, fragile X syndrome (FRAXA), fragile X-associated tremor/ataxia syndrome (FXTAS), Baratela-Scott syndrome (BSS), myotonic dystrophy (DM) type 1, myotonic dystrophy (DM) type 2, amyotrophic lateral sclerosis (ALS), Kennedy's disease, fragile X E mild mental retardation (FRAXE), progressive myoclonus epilepsy (EPM1), oculopharyngodistal myopathy (OPDM) type 1, oculopharyngodistal myopathy (OPDM) type 2, oculopharyngeal myopathy with leukoencephalopathy (OPML), frontotemporal dementia (FTD), Neuronal intranuclear inclusion disease (NIID), essential tremor, global developmental delay, progressive ataxia, and elevated glutamine (GDPAG), familial myoclonic epilepsy (FAME) type 1, familial myoclonic epilepsy (FAME) type 2, familial myoclonic epilepsy (FAME) type 3, familial myoclonic epilepsy (FAME) type 4, familial myoclonic epilepsy (FAME) type 6, familial myoclonic epilepsy (FAME) type 7, premature ovarian failure (FXPOF), cerebellar ataxia, neuropathy, vestibular areflexia syndrome (CANVAS), multiple system atrophy (MSA), refractory chronic cough, Fuchs endothelial corneal dystrophy (FECD), X-linked dystonia-parkinsonism (XDP), intellectual disability (FRA12A type), Jacobsen syndrome (CBL2), and any combination thereof.

32. (canceled)

33. A system comprising:
(a) an MSH3 pre-mRNA;
(b) a spliceosomal RNA; and
(c) an agent which modulates splicing of the MSH3 pre-mRNA such that an MSH3 mRNA transcript comprising the poison exon is produced.

34-54. (canceled)

55. An mRNA transcript comprising an MSH3 mRNA and at least one poison exon.

56-65. (canceled)

66. A method of screening for an agent that modulates splicing of an MSH3 pre-mRNA, the method comprising:
(a) contacting a cell with a candidate agent;
(b) detecting presence or absence of a poison exon in an MSH3 mRNA transcript; and
(c) identifying the candidate agent as an agent that modulates splicing of the MSH3 pre-mRNA when the poison exon is detected in the MSH3 mRNA transcript.

67-81. (canceled)

82. The method of claim 2, wherein the agent is a compound having the structure of Formula (I″), or a pharmaceutically acceptable salt or solvate thereof: 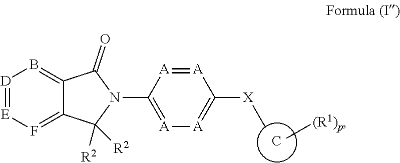
wherein,
ring C is 4 to 10-membered heterocycloalkyl or 4 to 10-membered heterocycloalkenylene;
each R¹ is independently hydrogen, halogen, —CN, —NO₂, —OH, —OR^{a}, —SH, —SW, —NR^{c}R^{d}, C₁-C₆alkyl, C₁-C₆haloalkyl, C₁-C₆hydroxyalkyl, or C₁-C₆aminoalkyl, wherein each of the alkyl, haloalkyl, hydroxyalkyl, or aminoalkyl is optionally substituted with 1 to 4 substituents independently selected from R^{e}; or
two R¹ are taken together to form an oxo; or
two R¹ are taken together to form a bond, C₁-C₃alkylene, or C₁-C₃heteroalkylene, wherein the alkylene or heteroalkylene is optionally substituted with 1 to 4 substituents independently selected from R^{e}; or
two R¹ are taken together to form a C₃-C₆cycloalkyl or 4 to 6-membered heterocycloalkyl, each of which is optionally substituted with 1 to 4 substituents independently selected from R^{e};
each R² is independently hydrogen, halogen, —OH, C₁-C₆alkyl, C₁-C₆haloalkyl, C₁-C₆hydroxyalkyl, or C₁-C₆aminoalkyl; or
X is absent, —CH₂—, —O—, —OCH₂—, —S—, or —NR³—;
R³ is hydrogen, C₁-C₃alkyl, C₁-C₃hydroxyalkyl, C₁-C₃haloalkyl, or C₃-C₆ cycloalkyl;
each A is independently N or CR⁴, wherein at least two of A are N;
each R⁴ is independently hydrogen, halogen, —CN, —NO₂, —OH, —OR^{a}, —SH, —SR^{a}, —NR^{c}R^{d}, C₁-C₆alkyl, C₁-C₆haloalkyl, C₁-C₆hydroxyalkyl, or C₁-C₆aminoalkyl;
or R³ and one of R⁴ are taken together to form a 5 or 6-membered heterocycloalkylene or 5 or 6-membered heteroarylene;
B is N or CR;
D is N or CR⁶;
E is N or CR⁷;
F is N or CR⁸;
R⁵, R⁶, R⁷ or R⁸ are each independently hydrogen, halogen, —CN, —NO₂, —OH, —OR^{a}, —OCH₂R^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, C₁-C₆aminoalkyl, C₃-C₆cycloalkyl, 4 to 6 membered heterocycloalkyl, 5 to 10-membered heteroaryl, or —O—(C₁-C₆alkylene)-5 to 10-membered heteroaryl; wherein each of the alkyl, alkene, alkyne, haloalkyl, hydroxyalkyl, aminoalkyl, cycloalkyl, heterocycloalkyl, or heteroaryl is optionally substituted with 1 to 4 substituents independently selected from R^{e};
wherein at least one of R⁵, R⁶, R⁷ or R⁸ is C₂-C₆alkene, C₃-C₆cycloalkyl, 4 to 6 membered heterocycloalkyl, 5 to 10-membered heteroaryl, or —O—(C₁-C₆alkylene)-5 to 10-membered heteroaryl, wherein the alkene, cycloalkyl, heterocycloalkyl and heteroaryl is optionally substituted with 1 to 4 substituents selected from R^{e};
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{e} is independently halogen, oxo, —CN, —OH, —S(═O)CH₃, —S(═O)₂CH₃, —CH₂S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6-membered heterocycloalkyl; and
p is an integer from 1-9.

83-101. (canceled)

102. The method of claim 82, wherein the compound has the structure of Formula (II′), or a pharmaceutically acceptable salt or solvate thereof: 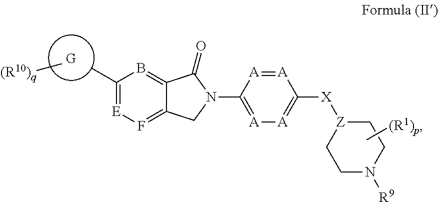
wherein,
ring G is a 5 to 10-membered heteroaryl;
each R¹ is independently hydrogen, halogen, —CN, —NO₂, —OH, —OR^{a}, —SH, —SR^{a}, —NR^{c}R^{d}, C₁-C₆alkyl, C₁-C₆haloalkyl, C₁-C₆hydroxyalkyl, or C₁-C₆aminoalkyl, wherein each of the alkyl, haloalkyl, hydroxyalkyl, or aminoalkyl is optionally substituted with 1 to 4 substituents independently selected from R^{e}; or
two R¹ are taken together to form an oxo; or
two R¹ are taken together to form a bond, C₁-C₃alkylene, or C₁-C₃heteroalkylene, wherein the alkylene or heteroalkylene is optionally substituted with 1 to 4 substituents independently selected from R^{e}; or
two R¹ are taken together to form a C₃-C₆cycloalkyl or 4 to 6-membered heterocycloalkyl, each of which is optionally substituted with 1 to 4 substituents independently selected from R^{e};
X is bond, —CH₂—, —O—, —S—, or —NR³—;
Z is CH or N, wherein when X is —O—, —S—, or —NR³—, then Z is CH;
R³ is hydrogen, C₁-C₃alkyl, C₁-C₃hydroxyalkyl, C₁-C₃haloalkyl, or C₃-C₆ cycloalkyl;
each A is independently N or CR⁴, wherein at least two of A are N;
each R⁴ is independently hydrogen, halogen, —CN, —NO₂, —OH, —OR^{a}, —SH, —SR^{a}, —NR^{c}R^{d}, C₁-C₆alkyl, C₁-C₆haloalkyl, C₁-C₆hydroxyalkyl, C₁-C₆aminoalkyl;
B is N or CR;
E is N or CR⁷;
F is N or CR⁸;
R⁵, R⁷, and R⁸ are each independently hydrogen, halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, or C₁-C₆aminoalkyl; wherein each of the alkyl, haloalkyl, alkene, alkyne, and hydroxyalkyl, and aminoalkyl is optionally substituted with 1 to 4 substituents independently selected from R^{e};
R⁹ is hydrogen, C₁-C₆alkyl, C₁-C₆haloalkyl, C₃-C₆cycloalkyl, or 4 to 6-membered heterocycloalkyl, wherein each alkyl, haloalkyl, cycloalkyl and heterocycloalkyl is independently substituted with 1 to 4 substituents selected from R^{e};
each R¹⁰ is independently halogen, —CN, —NO₂, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₃-C₆cycloalkyl, or 4 to 6-membered heterocycloalkyl, wherein each of the alkyl, haloalkyl, hydroxyalkyl, aminoalkyl, cycloalkyl and heterocycloalkyl is optionally substituted with 1 to 4-substituents independently selected from R^{e}; or
two R¹⁰ are taken together to form an oxo;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{e} is independently halogen, oxo, —CN, —OH, —S(═O)CH₃, —S(═O)₂CH₃, —CH₂S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6-membered heteroaryl; and
p is 8;
q is 0, 1, 2, or 3.

103-109. (canceled)

110. The method of claim 102, wherein the compound has the structure of Formula (III′), or a pharmaceutically acceptable salt or solvate thereof: 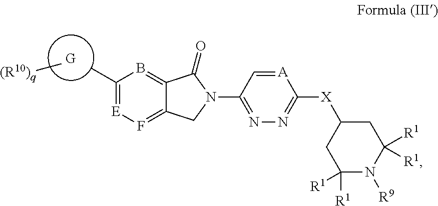
wherein,
ring G is a 5 to 10-membered heteroaryl;
each R¹ is independently hydrogen, halogen, —CN, —NO₂, —OH, —OR^{a}, —SH, —SR^{a}, —NR^{c}R^{d}, C₁-C₆alkyl, C₁-C₆haloalkyl, C₁-C₆hydroxyalkyl, or C₁-C₆aminoalkyl, wherein each of the alkyl, haloalkyl, hydroxyalkyl, or aminoalkyl is optionally substituted with 1 to 4 substituents independently selected from R^{e}; or
two R¹ are taken together to form a bond, C₁-C₃alkylene, or C₁-C₃heteroalkylene, wherein the alkylene or heteroalkylene is optionally substituted with 1 to 4 substituents independently selected from R^{e}; or
two R¹ are taken together to form a C₃-C₆cycloalkyl or 4 to 6-membered heterocycloalkyl, each of which is optionally substituted with 1 to 4 substituents independently selected from R^{e};
X is —CH₂—, —O—, —S—, or —NR³—;
R³ is hydrogen, C₁-C₃alkyl, C₁-C₃hydroxyalkyl, C₁-C₃haloalkyl, or C₃-C₆ cycloalkyl;
A is N or CH;
B is N or CR;
E is N or CR⁷;
F is N or CR⁸;
R⁵, R⁷, and R⁸ are each independently hydrogen, halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, or C₁-C₆aminoalkyl; wherein each of the alkyl, alkene, alkyne, haloalkyl, and hydroxyalkyl, and aminoalkyl is optionally substituted with 1 to 4 substituents independently selected from R^{e};
R⁹ is hydrogen, C₁-C₆alkyl, C₁-C₆haloalkyl, C₃-C₆cycloalkyl, or 4 to 6-membered heterocycloalkyl, wherein each alkyl, haloalkyl, cycloalkyl and heterocycloalkyl is independently substituted with 1 to 4 substituents selected from R^{e};
each R¹⁰ is independently halogen, —CN, —NO₂, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₃-C₆cycloalkyl, or 4 to 6-membered heterocycloalkyl, wherein each of the alkyl, haloalkyl, hydroxyalkyl, aminoalkyl, cycloalkyl and heterocycloalkyl is optionally substituted with 1 to 4-substituents independently selected from R^{e}; or
two R¹⁰ are taken together to form an oxo;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{e} is independently halogen, oxo, —CN, —OH, —S(═O)CH₃, —S(═O)₂CH₃, —CH₂S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6-membered heterocycloalkyl; and
q is 0, 1, 2, or 3.

111-112. (canceled)

113. The method of claim 110, wherein the compound of Formula (III′) has the structure of Formula (IIIa′), or a pharmaceutically acceptable salt or solvate thereof: 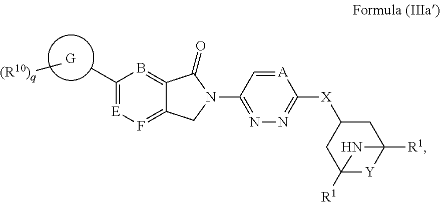
wherein
Y is C₁-C₃alkylene;
X is —O— or —NR³—;
(i) R³ is hydrogen, C₁-C₃alkyl or C₃-C₆ cycloalkyl:
each R¹ is independently hydrogen, halogen, C₁-C₆alkyl, or C₁-C₆haloalkyl, wherein each of the alkyl, haloalkyl, hydroxyalkyl, or aminoalkyl is optionally substituted with 1 to 4 substituents independently selected from R^{e};
each R^{e} is independently —F, —C₁, —OH, —CH₂S(═O)₂CH₃, methyl, ethyl, isopropyl, tertbutyl, —OCH₃, cyclopropyl, cyclobutyl, cyclopentyl, cyclohexyl, or azetidine, oxytane, piperidine, piperazine, or morpholine; or
(ii) R³ is hydrogen, methyl, ethyl, or cyclopropyl, and
each R¹ is independently hydrogen, halogen, or C₁-C₆alkyl.

114. (canceled)

115. The method of claim 110, wherein the compound of Formula (III′) has the structure of Formula (IIIb′), or a pharmaceutically acceptable salt or solvate thereof: 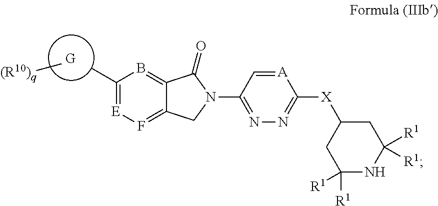
X is —O— or —NR³—;
(i) R³ is hydrogen, C₁-C₃alkyl or C₃-C₆cycloalkyl;
each R¹ is independently hydrogen, halogen, C₁-C₆alkyl, or C₁-C₆haloalkyl, wherein each of the alkyl, haloalkyl, hydroxyalkyl, or aminoalkyl is optionally substituted with 1 to 4 substituents independently selected from R^{e};
each R^{e} is independently —F, —C₁, —OH, —CH₂S(═O)₂CH₃, methyl, ethyl, isopropyl, tertbutyl, —OCH₃, cyclopropyl, cyclobutyl, cyclopentyl, cyclohexyl, or azetidine, oxytane, piperidine, piperazine, or morpholine; or
(ii) R³ is hydrogen, methyl, ethyl, or cyclopropyl, and
each R¹ is independently hydrogen, halogen, or C₁-C₆alkyl;
optionally, two R¹ are taken together to form a C₃-C₆cycloalkyl.

116-126. (canceled)

127. The method of claim 102, wherein the compound has the structure of Formula (IV′), or a pharmaceutically acceptable salt or solvate thereof: 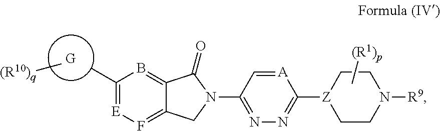
wherein,
ring G is a 5 to 10-membered heteroaryl;
each R¹ is independently hydrogen, halogen, —CN, —NO₂, —OH, —OR^{a}, —SH, —SW, —NR^{c}R^{d}, C₁-C₆alkyl, C₁-C₆haloalkyl, C₁-C₆hydroxyalkyl, or C₁-C₆aminoalkyl, wherein each of the alkyl, haloalkyl, hydroxyalkyl, or aminoalkyl is optionally substituted with 1 to 4 substituents independently selected from R^{e}; or
two R¹ are taken together to form an oxo; or
two R¹ are taken together to form a bond, C₁-C₃alkylene, or C₁-C₃heteroalkylene, wherein the alkylene or heteroalkylene is optionally substituted with 1 to 4 substituents independently selected from R^{e}; or
two R¹ are taken together to form a C₃-C₆cycloalkyl or 4 to 6-membered heterocycloalkyl, each of which is optionally substituted with 1 to 4 substituents independently selected from R^{e};
Z is CH or N;
A is N or CH;
B is N or CR;
E is N or CR⁷;
F is N or CR⁸;
R⁵, R⁷, and R⁸ are each independently hydrogen, halogen, —CN, —NO₂, —OH, —OR^{a}, —SH, —SR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₂-C₆alkene, C₂-C₆alkyne, C₁-C₆haloalkyl, C₁-C₆hydroxyalkyl, or C₁-C₆aminoalkyl; wherein each of the alkyl, alkene, alkyne, haloalkyl, and hydroxyalkyl, and aminoalkyl is optionally substituted with 1 to 4 substituents independently selected from R^{e};
R⁹ is hydrogen, C₁-C₆alkyl, C₁-C₆haloalkyl, C₃-C₆cycloalkyl, or 4 to 6-membered heterocycloalkyl, wherein each alkyl, haloalkyl, cycloalkyl and heterocycloalkyl is independently substituted with 1 to 4 substituents selected from R^{e};
each R¹⁰ is independently halogen, —CN, —NO₂, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₃-C₆cycloalkyl, or 4 to 6-membered heterocycloalkyl, wherein each of the alkyl, haloalkyl, hydroxyalkyl, aminoalkyl, cycloalkyl and heterocycloalkyl is optionally substituted with 1 to 4-substituents independently selected from R^{e}; or
two R¹⁰ are taken together to form an oxo;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{e} is independently halogen, oxo, —CN, —OH, —S(═O)CH₃, —S(═O)₂CH₃, —CH₂S(═O)₂CH₃, —S(═O)₂NH₂, —S(═O)₂NHCH₃, —S(═O)₂N(CH₃)₂, —C(═O)NH₂, —C(═O)NHCH₃, —C(═O)N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6-membered heterocycloalkyl;
p is 8; and
q is 0, 1, 2, or 3.

128-136. (canceled)

137. The method of claim 110, wherein
A is CH;
B is N or CH;
F is N or CR⁸;
R⁸ is hydrogen, halogen, C₁-C₆alkyl, or C₁-C₆haloalkyl;
E is N or CR⁷; and
R⁷ is hydrogen, halogen, C₁-C₆alkyl, or C₁-C₆haloalkyl.

138-151. (canceled)

152. The method of claim 137, wherein ring G a 5 to 6-membered heteroaryl optional substituted with 1 or 4 substituents selected from R^{e}.

153. (canceled)

154. The method of claim 152, wherein ring G is pyrrole, imidazole, triazole, tetrazole, thiophene, oxazole, thiazole, or thiadiazole.

155. The method of claim 137, wherein ring G is: 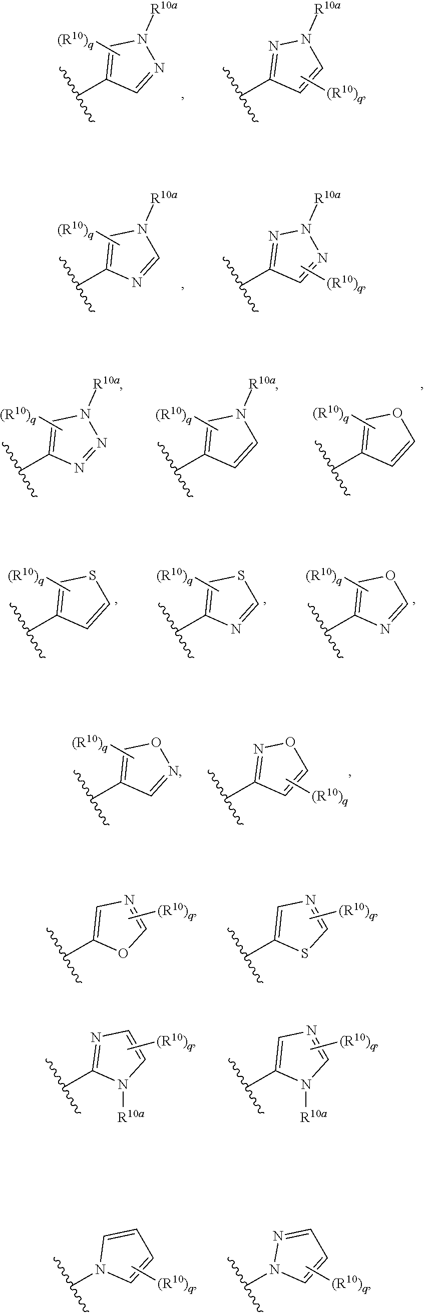 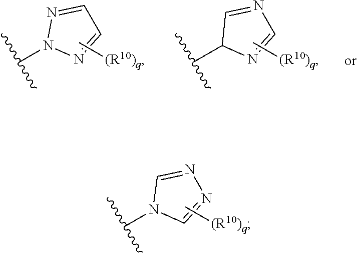
wherein,
each R¹⁰ is independently halogen, —CN, —NO₂, —OH, —OR^{a}, —NR^{c}R^{d}, C₁-C₆alkyl, C₁-C₆haloalkyl, C₁-C₆hydroxyalkyl, C₁-C₆aminoalkyl, C₃-C₆cycloalkyl, or 4 to 6-membered heterocycloalkyl wherein each of the alkyl, haloalkyl, hydroxyalkyl, aminoalkyl, cycloalkyl and heterocycloalkyl is optionally substituted with 1 to 4-substituents independently selected from R^{e};
each R^{10a} is independently hydrogen, C₁-C₆alkyl, C₁-C₆haloalkyl, C₃-C₆cycloalkyl, or 4 to 6-membered heterocycloalkyl, wherein each of the alkyl, haloalkyl, cycloalkyl and heterocycloalkyl is optionally substituted with 1 to 4-substituents independently selected from R^{e}; and
q is 0, 1, 2, or 3.

156-163. (canceled)

164. The method of claim 137, wherein ring G is: 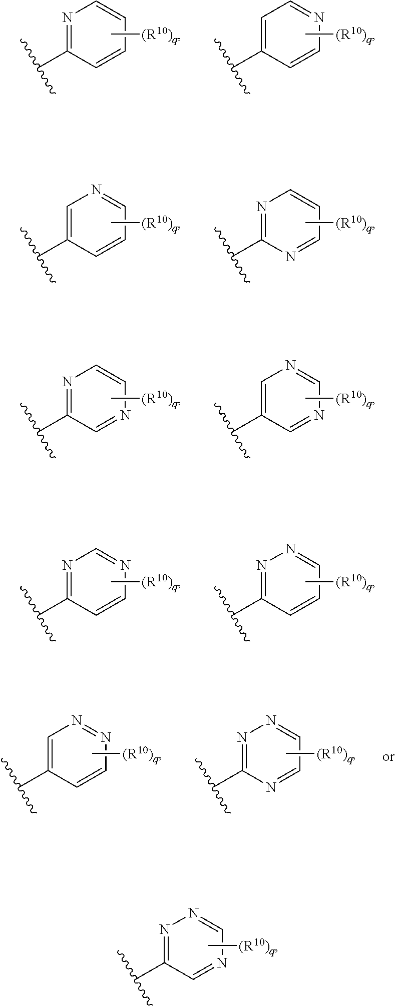
wherein,
each R¹⁰ is independently halogen, —CN, —NO₂, —OH, —OR^{a}, —NR^{c}R^{d}, C₁-C₆alkyl, C₁-C₆haloalkyl, C₁-C₆hydroxyalkyl, C₁-C₆aminoalkyl, C₃-C₆cycloalkyl, or 4 to 6-membered heterocycloalkyl, wherein each of the alkyl, haloalkyl, hydroxyalkyl, aminoalkyl, cycloalkyl and heterocycloalkyl is optionally substituted with 1 to 4-substituents independently selected from R^{e}; and
q is 0, 1, 2, or 3.

165-168. (canceled)

169. The method of claim 155, wherein:
each R^{a} is independently C₁-C₆alkyl, C₁-C₆haloalkyl, or C₁-C₆hydroxyalkyl, wherein each of the alkyl, haloalkyl, and hydroxyalkyl is optionally substituted with 1 to 4 substituents independently selected from R^{e};
each R^{b} is independently hydrogen, C₁-C₆alkyl, C₁-C₆haloalkyl, or C₁-C₆hydroxyalkyl, wherein each of the alkyl, haloalkyl, and hydroxyalkyl is optionally substituted with 1 to 4 substituents independently selected from R^{e}; and
R^{c} and R^{d} are each independently hydrogen, C₁-C₆alkyl, or C₁-C₆haloalkyl, wherein each of the alkyl, and haloalkyl is optionally substituted with 1 to 4 substituents independently selected from R^{e}.

170. The method of claim 169, wherein:
each R^{a} is independently C₁-C₆alkyl;
each R^{b} is independently hydrogen or C₁-C₆alkyl; and
R^{c} and R^{d} are each independently hydrogen or C₁-C₆alkyl.

171-175. (canceled)

176. The method of claim 2, wherein the compound is selected from the group consisting of: 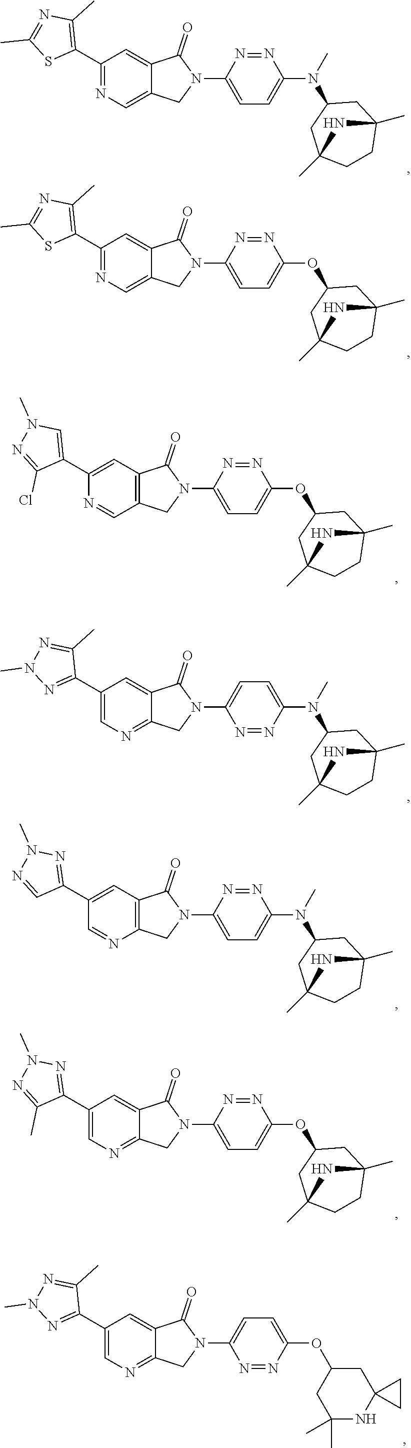 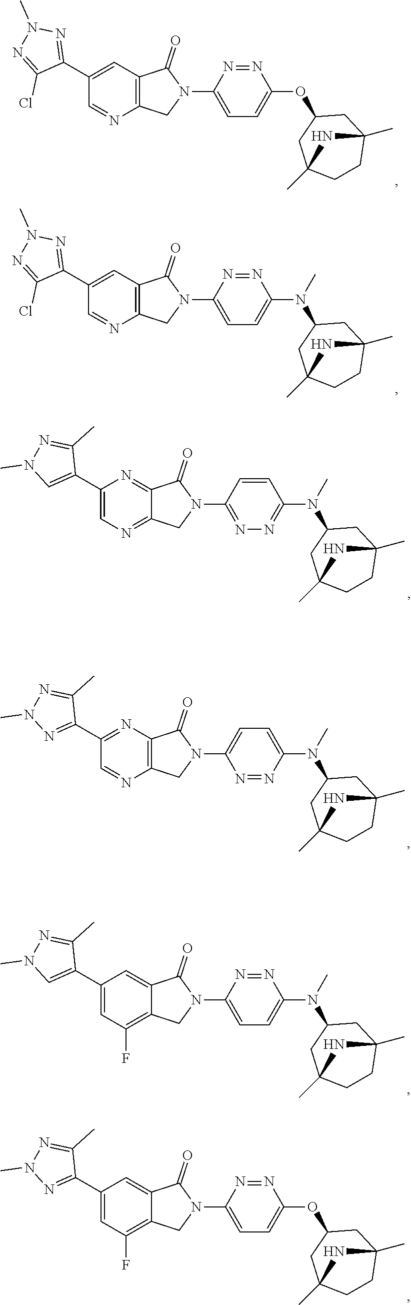 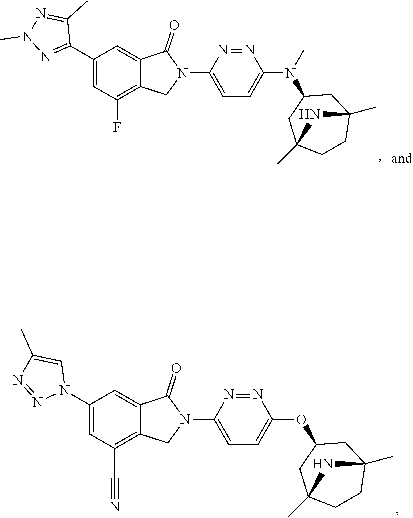
or a pharmaceutically acceptable salt or solvate thereof.

177. (canceled)


SEQUENCE LISTING (25 sequences)

SEQ ID NO: 1 - DNA, 94 bp, Homo sapiens
  source 1..94: mol_type = genomic DNA
cattttccttgcaaaaggcccagagattgtgatttgaagggacagaagcccactccatttgcctgagagacatctagaatattatctcaaaata

SEQ ID NO: 2 - DNA, 148 bp, Homo sapiens
  source 1..148: mol_type = genomic DNA
agttttactagaatgataagtactgcttctaagaatacttgaggtgcccaagaggacttacgtaactgttttacatgcctcagctaaatttgaaaacaatgcacaagccagaactggaaatactgatcctggggagaaagatatatga

SEQ ID NO: 3 - DNA, 4711 bp, Homo sapiens
  source 1..4711: mol_type = genomic DNA
agttttactagaatgataagtactgcttctaagaatacttgaggtgcccaagaggacttacgtaactgttttacatgcctcagctaaatttgaaaacaatgcacaagccagaactggaaatactgatcctggggagaaagatatatgagttggttatggggccggggagcctattgcatcattattgggtacaccatgttattgttttgctggcaatagcagaagctgtgctgatagtcgcaagttttatggtagacctttttgaagttgcaatgtgggtgccaaaccatttgttgtttttatccttggctcagagcctgaaaattgatgcatattaaccttctttaaacaacagatctccaggcaccttgccttaactacttcaaaggattcactgacagatgtcagagatttgaaaacagaagcaaagctgaaaatctttcagtggctactcaaatgcacaaagtcaaagtgtccatggcatcaagatggcagcatcagttcatggggctgtgatttctattccaccttcctgcctttttgtaaactttactttgatgtttgtattgcacagggtttgtgtatttataaaagtttcaaaactctagtaaagtcagaatctaattcatttaaaaagtggccggtatgcagtgcaagaatgacttattttggaatttcaaaatatagtttactaacggaacttataactcacttggaagccattccttgttgctgttttagtgtttgtttacacaagaatgtacctaaagattagtttgaagggtgaagagtagaaatgatctggtaacaaaccaacttcaggaaagctgaatgctttaaaccaatgttttatagattagttggctggctctgtgtacacaccatcaaacatatgtctgttctttattattagattactctaaataagcttctactgtgtacaccacatagtgggtgatgtcctgggtacagattgtgccaattatgtgaaattcataaaatgagaaggaacactggatgttgtgggaagtggttagcataggctgtatggttttgtggaacgtgccctggaatatgtaccaataggtgtttttatggtgataatgatagaggatctcaagtgagctgattctggtataaaatagacatttcttcttcttctttttttttttgaaacaagatctcactgtgtcacccatgctggaatgcagtggtgcgaccacagctcactacagcttcagcctccagccttctgagtagttgggacaacaggcacgtgccaccatgcccagctatttttttttttttttttgcatttttttgtagagacaaggtgttgccatgttgcccaggctggtctccagctcctgggttcaagagatcctcccaccttggactcccagtgtgttgggattacgggtgtgagccactgtgcttagcctaaaataaacatttcttttgatggcaaaatgttaaattcaaatcctgtaaatagtgctagtcacaggaacccataataactttgctcaagaactgttttgtaagaaattcttaagtaactgccgaggttggtgaatacatttaaaggataatcgcacttagcaaatgttgaggagagtgttttcagaatttcctaggtgaaaatctttttttagctaagcaggaataagcccacagctgcatgttcagtgtggcacttaagctggggtccttcccatcctgtgtccagtgaggccctgccaccatacttgtgggtctgtgctcaagctctcccagtgagacagctgctgccagaagtagggctgaactcctggagagtgacttcaggagcagccacttacccatcctcagctttcttgtagcttcttttgagttcttttgcttaagaaacttgagaacaacctgctgcctctggctttcaacatgattgtaaagcactactggttttcaccttgcttctgttgctgacttaactttacattgtgtctcagcttggaaaattaatcattttcttgggattacaataatcataagaggttaatattcaaaattatattgaaaatgtttgtgtttgtttctttacttttgttaaaaggtatcattaattgactagtggccaaaaagcttccaggtttcacatttatttaaacagtccagcttcctgtatagggtggggagggggtctttctggagaaaagttgcctcatgatacacttagaattcaacatttgctcagttggacctaacttcttattttttttgcataattcaccctttgtggttagtaattacctttttgaatacgttcttagccttaatctatttcatatatactatctattatgtgccttattactttacccctaaattgttttctcatctcaagacagttcttttcagccaacattttgtcagttttattttcctgagctgtcttctagagccttctaacttgatccagtctggactggttgtccccatatctgttgtatagtactatcttagattccctttacttcccttgagttgaatcttttattttctatattccatgtcttcctcttcgtttatttcttcattttggtggagtatatatcctctagaaccttcctgagaaagcacgttggaagtaaatcttttgaaacattcatgtctgaaagtggtttttttgttgttgttattattgttactaccttcacatttgattggtagtttggattggtataaaattctaggttttagatgacttcattatctttacttcctcttctgttgaatttttcatttctactattatgctttaatttccaagaatttttgttttcttgtcctccaaatgttttttctttttaaaagtagcatcttgtttcatggttgtagtatcttctgtttcatggaagatattattaatatttttagaagttccctaacagggcagaatccatattggtacacaagtctttttggattcttgatgtaaattgtgtctccgttttccttaaggacagtttaaaattcagataccttgagtcttttaagtcaattactgtttatttgtttactttatagcttctaacatattattgctgttgtcttcttttctagtgtgtttttttccttttttttttttttttttttttttgagacggaatcttgctctgttgcccaggcaggagtgcagtggcacgatctcggctcactgcagtctccgccttccaggctcaagcgattctcctgcctcgccacctgagtcactgggatcacaggcgagcgccaccacacccagctaatttttgtatttttagtagagatggggtttcaccatgttggtagggctggtctcgaactcctgaccttgtgatctgcccgcctcagcctcccaaagtgctgggattacaggcgtgagtcactgcgtctggctggttttatgccgttttaaacaatctcttaactgtcgtttcaagatggagagaaattagatgcatgtgtttaactcactgtttaccagaagctatgatataaaaattattcatgttcatgttaaaaaaaaaactacagtagtatgcaggggtatgcagtaaagggtaaaaagtctccctctgccacccttccatacctactacccagaggtaaccagagtaagtcacatgtgttacctcctaaaatttttattgatatttagattttttatttatatagaataatgtatatcatttaaagaatacaaagaaagtgttaccatgtaaatacataaaaatatatacatatatggtatatgatataaacatactgtatatattcaatgtatgttgaaattatggagttctatactgatgcaaacagatcttaattttcttttagctgtttcgtagtactacactgcatgagtttttcccaataagatcccaattgatggacatttatttcttattcatctttttaaattataagcagcgctactatgaatattcagacacacatatttttcactatttgttagtctatttttagataaattatttaaagtggtgcttattcaaagggttatgtgcacttttttattacgaagacatgccaagttgtttttcaaaaaggttagtcctctgctaagagtatataaaagtgtcttttttttcttttacatcctgttacatggttattgtgaagcttttaaatctttggcgttccgataagtgaaaaatgctatcctatatttctaatttgcttgtctttaattatatatgaagataatcgtatacatacttgctgttctctttctatgagctgcatattcagtttgtttagccattgttctttggattattcatctttttcttgtcaatttgtaagagcctattatgtttgggagaattttgccttgtgtctgttatgccttacagatattatccttcacattttttaacttgtttaacctatttttgctatatggtttatgttattttaaaaatgaaaaaatactaaacaagtattggctttgttttccttgcctaaaattatttcctttatacctttaaaatattacatagtatttaacctcatttagaaaatatcagtgatattttatttcaactaactagttaatagaaaatttaaactttcctcaattctgattctaacttttaaaagtcactaattaaacttcattttcatgtatcttatgctatttcataaaaaatatttctattttcag

SEQ ID NO: 4 - DNA, 10 bp, Homo sapiens
  source 1..10: mol_type = genomic DNA
atgagttggt

SEQ ID NO: 5 - DNA, 10 bp, Homo sapiens
  source 1..10: mol_type = genomic DNA
ttgggtacac

SEQ ID NO: 6 - DNA, 10 bp, Homo sapiens
  source 1..10: mol_type = genomic DNA
aaaggttagt

SEQ ID NO: 7 - DNA, 10 bp, Homo sapiens
  source 1..10: mol_type = genomic DNA
aatagtaaga

SEQ ID NO: 8 - DNA, 10 bp, Homo sapiens
  source 1..10: mol_type = genomic DNA
ttgagtaagt

SEQ ID NO: 9 - DNA, 10 bp, Homo sapiens
  source 1..10: mol_type = genomic DNA
gcaggtaaag

SEQ ID NO: 10 - DNA, 10 bp, Homo sapiens
  source 1..10: mol_type = genomic DNA
cttggtatgt

SEQ ID NO: 11 - DNA, 10 bp, Homo sapiens
  source 1..10: mol_type = genomic DNA
aaaggtacat

SEQ ID NO: 12 - RNA, 11 nt, synthetic construct
  source 1..11: mol_type = other RNA
atacttacctg

SEQ ID NO: 13 - DNA, 12 bp, unidentified
  source 1..12: mol_type = unassigned DNA
ccccgccccgcg

SEQ ID NO: 14 - DNA, 22 bp, synthetic construct
  source 1..22: mol_type = other DNA
cgtaaattgcccgacatagaga

SEQ ID NO: 15 - DNA, 23 bp, synthetic construct
  source 1..23: mol_type = other DNA
aacagttacgtaagtcctcttgg

SEQ ID NO: 16 - DNA, 26 bp, synthetic construct
  source 1..26: mol_type = other DNA
agcatttatcacaaaaaaagttttac

SEQ ID NO: 17 - DNA, 22 bp, synthetic construct
  source 1..22: mol_type = other DNA
ctcagattggctcctatgttcc

SEQ ID NO: 18 - DNA, 20 bp, synthetic construct
  source 1..20: mol_type = other DNA
gggcttctgtcccttcaaat

SEQ ID NO: 19 - DNA, 21 bp, synthetic construct
  source 1..21: mol_type = other DNA
ttcacaagcattttccttgca

SEQ ID NO: 20 - DNA, 21 bp, synthetic construct
  source 1..21: mol_type = other DNA
aaataaatgcccggcttgatg

SEQ ID NO: 21 - DNA, 25 bp, synthetic construct
  source 1..25: mol_type = other DNA
ggcttgtgcattgttttcaaattta

SEQ ID NO: 22 - DNA, 25 bp, synthetic construct
  source 1..25: mol_type = other DNA
taaatttgaaaacaatgcacaagcc

SEQ ID NO: 23 - DNA, 25 bp, synthetic construct
  source 1..25: mol_type = other DNA
agcaggtattattgcttgaaattct

SEQ ID NO: 24 - DNA, 20 bp, synthetic construct
  source 1..20: mol_type = other DNA
ccactaggcgctcactgttc

SEQ ID NO: 25 - DNA, 18 bp, synthetic construct
  source 1..18: mol_type = other DNA
acccgttgactccgacct